SEQUENCE LISTING (60 sequences)

SEQ ID NO: 1 - DNA, 59558 bp, Cricetulus griseus
  misc_feature (1)..(30020): CHO genome outside of ERV on 5′ end ERV-C 109F
  misc_feature (30021)..(39247): ERV type-C, LTR to LTRERV-C 109F
  misc_feature (39248)..(59558): CHO genome outside of ERV on 3′ end ERV-C 109F
tggttctatcgagacagctacatccgcttctccacacagcccttctccctgaagaacctggacaagtgagctttccctccacctcccttgactgcccaggctagtgaggaggtcagcagacaaacagaacagtgggctctgcgggcagggaaggcaggcttttcgccgccactcatcctctgccctgaaggagcttgccaagagggtgcccttgggttaccgaaatgagcaaaacaagaactcctgctaatcaagggcttacatcctagcaatgagcttgagtaatcatggctaacatgctagaaggtgatatgtgttaggaagaaaatgtagatctgggtaatggcagattaagagaaacttgagaggggaaaggcaacttgcagtttgctcttgggatgagaggaaaccttgaggagacctgggttcaaattctatctctgcagcatccagattgtgttcctctagagacatgaaaagagacttaagcctcagtttcagttagagcccagtggtcccatctgtaatcccagcacttgggagtctgagtctgaaggattttgaggtcattctgggctatataatgagacccccactcaagacaaaacaagccaggtgtggtagctcctaactgtaatcctagaacttggctaaggcaggagccaaaatcaagccaggtacggtattggaggcccgtgatcccagtgcttagaaggtagaggcaggacaatcagttcactgtcaccctcagctacatgccaagttttatctaacctgggcaacatgagaccatttcatagaaaaagaccagcctccgtttccacatctgaagactggagcagcaatacactgtagctgcttatcatcaagtacttcacatgtgataagttgttcctgcttggttataccacagccacttctcaatggctcacaatgaagtgagttattgccattttaaaaagagatgagaggggcaaaggttaaatgacttgcctgagatctcatagttgatcatcggtgaagctgggatttgaccatccagggtcatcgtccccctgccacacataggaatggtgaactgaaactaggcccaaagcacttgatacttactgtggcctgcaggcaacactggagcattctgagaaatgactagccctggccgtgggtgaatagggagtggggcagggagtgggggtggggtggaggggtgcagcttctgtgcttctgtcctgttgatcttccagcaggggcccctctcagttgagatcccctagacggctgaccacagggttctgcctgcagccctgaggttgggctccagggctgagcctgtgtctctgatccactctagctctgtgcacctatgtaacaattccatccagagatacctggagacctcctgtcaccggcaccggatgctgccctcggacaacatgtggtccagccagaggtttcaggcccacctgcaggaaatgggtgccccaaatgcctggtctagtgtcattgtacccggcatgaaggctgctgtgatccatgccctgcagacctcccaagacactgtgcagtgccgaaaggccagctttgagctctatggggccgactttgtgtttggggaagacttccggccctggttgattgagatcaatgccagccccaccatggcaccttccacagctgtaactgcccgcctgtgtgctggtgtgcaagcagataccctccgtgtggtcattgaccggcgactggaccgtacctgtgacacgggagcctttgagctcatctataagcaggtgagatgtcccagcacctcccacaggcaaccctacagcaaagccctggctggggtctgctgtgagacagagttcaagactgactctacacacggggcatcttaacacagacacgtcccactggcctgtctcctcatctgtggaagatactgtcctttgagagccattaatgccatgagtttgtagtcataggtagtattttcagaggccctgggacctgctcagtttccatggtaattccaagcctctaggtagtaaccttattctctcatctgtaaagtaagactgtacctggctcctcctttgtgtgctgtgagaatgggtggtttgatcttcacacaagggtttgctaaagtaccaagctggaggacatagaggatatgaggccatgaacctcaaggcctgcctatcaagagttaattattagtgtctatcattgttataacgattattatgttactcatcttcttccaaaaacagatttgatcttgcttatttataataagtatagaattgcttggggtttttgtttttgtttttgtgggttttgttgagatagggtttttctgtatagctttggatcctgttctggaacttgctctgtagaccaggctggccttgaactcacctttgactgcctctgcctcccgagtgctgggactaaaggtgtgcaccaccattgcccgacctagaattgttttttattcagccaaaaaacattaccttgccctcgtggtctaattctctctagaaacacccttaggagcacaccaggggcatgccctatagaaatcctaggttttctcagtccagtctagttgacaactgatattagccaccacagctggacatggggctccaccttccacctcccagtatttggaaggctgagacaggggatcacttttagttcaagggaagcttgggttacacagtgagttccaggctagcctgggcttcacagtgagaccctacttaaaaacatcatacaaacaaacaaacaaacaaacctttaaacgtatgtttttaagggtttctgaattctgaggacagtttttaagatctttgagctaagattcaccaaggtctaggtttgctacaggaagggaaaacactgatcacctgacctgggtggcctcatgtttcctacaggtcctgatcaccttagaataatgtgctggcaaagggttccgtctttgttagggatgccatcactaggtgtccagggtggaatccaggcctgcgtttttagcatgtgcagttctactgagctacacctccagcctaaaaaatgtaaaggaggagatgcatgcaggtgtgcatacacctgtaatccctgggcaacagaagcaagaggactgtcacaggttcaccaccacctggttacagggtttataataaggtcctgtcacaaacaatgtagtcttggaagagaggagaggaaatggggaagaggaagtaatttgcagtttaaaatagagcaaaattgccgggcgttggtggtgcacacctttaatcccagcactcgggaggcagaggcaggcgcatctctgtgaattcgagactagcatggtctacaagagctagtttcaggacagcctccagagctacagagacaccctgtctcaaaaaaaaaaaaaaaaaaaaaaaaaaagcgaaatttgtatggacatggcaacacgtgcctgtaatcccaacacttagggggctgaggttggaggatcaggagttcaaagttatccttggctgtatgtgagcttgaagccaacctgggcctacagcactgtctgttccacaatctgtctttatctgtttgtctccttatgttgttcagctcggtctgttctctgaatgtttgtctcagaacaaacaaaattgaatagggctgggcatacgcacttttcaaatgtcctttgtcccccaggtatctttcattgctgatttggttttgagcttgagggtcagtgtacaacaaggtggttacaatggtggctttgtctgtctctgatctcctttatctaggacagtgccactgctgtatccctggcaccctggtcctttgcaggccaggcaggccagcctgcaccaccgccccacactgttttatctgtttttctccttatgttgttcagatcggtctgttctctgaatgccctgtaaactagaagataggctaacaagctgatggggttcagggttgagatttttggcaaaaaacactcatgtgatgctaggtacctcatgtgactgtcacaaggcacaaccaggaatctctagttcccaatgccgaatttgaccttagactaaggtggctgccaccagagccacatcctcccctttgtagcttttttcatttttctttacattatttattgtgtttgagtttgtacatgtgtgtggtcacacatgccacagcacacatgtgggagtcagagggcaacttatgggagttggttctctcctcccatcccatgggtcctggggcttgaactcagcaagtaccttataagctatctcaatactgtttgcctccaaaatgtattactttgtgtagctgtcccttttctgttgctaataacagaatactacagacagtgtaagttacaaagaaaataggctcaattgtatccatcttttgctgcccatggcatgaaaacacttgcatcccaacacaggccagaggtcgctttagtgtgaagcaggattgagtgcatgtctgcatggctaagactctctacttctctggtttcctgatccctagggctctgggactctagatcctctggaccccttgataatagagggagcttcctgtttctcagaagtgccttttgaccatatatcccagaaactgattccatccatctctgccgtgtgtcactagtcactagatggcgtcactgtcatctctcactagtgtctgagatggccttgtcttctctcctgcccacagcctgctgtggaggtgccccagtacgtggggatccggctcatggtggagggctctaccatcaagaagcccatagcagcttgtcatcggcggacagcggtccgctcatcactccctcatctgctggcccagcaaggctgtggggaaggcaaggactcaggaccccctatccacaggtcagcttctaggaaagatgctggggccaggagcctgggacacactgagaagccagactctgcggccaccacctcagtccccggaaaggggaagaaaggcaaggcaaaaagtgccacagccctggtctgcatcaccctgcagaaatgggagtcccacaacaccagggtgggccccaccttcaacaggttaatgtgtctgaaacagcctgaggcctggggtagtaccatgtcccccaaaccccgcagtgttcccaaggccatttctgcctgctctccaagccctccccaagcatctgggcttgccctcctgccaaaaggccaccagtgatagcaagtatgaaccaaatatctttaaatacataaccaaatgagtattacaaagtagtcaccctgccaggcagttagaccaaaggctcggtcctagagtgcgcgcccagagtccagacccatgctgctgctctagccagcctttgccctcacctttctctggagaaaggtgctgccaccatgcccttccccattcctaaccagccccctcagccctcataacgccctagtgaggtaggtgctattgtccccattttccagccgaggtagcagcaagtttaaggacgttgcccgaggttgcacagctcagaaggggcagagctgggatgcagacccaggtctgttggtctcccaaccctgtgttcttcccactgcctctggaggaggagctgggaggggctccatctgcccttaccttgtatcccccacctttacacatgtactgtggaacaattggtcaggctggggcctcacccagatcctcacagcttcccttctcccacagccccgtcctacctccctagtctccattccaaggcctggctgccttcttcccatgtgctccgaccccagggccgggtcctcagactaccgaatggccaactggtgggctctaaggctctgtcaaccacaggcaaggccttgatgactctacctactgccaaggttctgatgtccttcccacctcaccctgatctcaagctggcacccagcatgctgaagccaggaaaggtgggcctcgagctgtgcctcacaccctggcgggtagtgctgagcagtgggatcggggctgaagggcacgaacagagggcagcgctcggaccatacagcgccccagggaagggcttgtcttctccagaaccctgttccaagacagaggcctgatcatatctctttccctcccctccttgcaccgaggctgctattcccctgcaccttcgaggcccccactttggaagtgcctcgaggcctctgccctttgaagttggacctcttcctagcacccacaggaaagtcacggccaaaggcaagttcaaggccatactctgcgacaaagccagggctgaggcataccccaagaagaggctgagcctccccaaacccttgacccttattctgacatgccggacactgagaaccatgggggataggaggctagagaaacccctgctctgatctctactgccccatcctggatccagcatcaaattaaaaaaagcaattaaagttctctggacttggcttgaataatgtgcggctaggctcataaaagagttgaccagcagggcctccatcagcaagggccacagtccccacccagcgacagacattggctttctctgcagggagacggatgggtggggaaagagccttcactatacagatgatgacactgagacatggcttgcctgagaccacagcaggcggaaagtcagccatcaggagtgccccttcccaaagacaagctgggctggcagaagagcttctgatggtcacagaacattatgacaagagacgggctttccctaaacctcagcctttctccggagagtatgtccctcagtgaggggtcggtcaagacacctcaaactgcaaaacccaagaaacaggtcaagtgtggcatttcctacctgtagagcctcagctgctggtcctccaaaagcggtatctagctgtagcgtgtgaatgcttcctgagtggggcagggtggagaggagagtgcggtgttgaaatccaatgacctgtgtctcccaaagtcagaagagctcatgcctgcacagtggtgtgtgtctgtcctcccaggacttgggaggcagaggcaggtgcatctctgaattccagtccagccagggatataccaagaggcccagcctcaaaagcaaacaaacctcctcgtgaccaggagtacagcagatgccacctccagacctggccactgttcacttgagcagagagcacagtccctggtcaacacttgctttctcagcagatcctcaaaagcagacttgtgaggtaggacttttattattttaagtccagagagcggtcacctgcccaacgccacacagtaagtgagtggtttaactgggatttatcttaggttggtaggactaaggagtcaaagaccttgaccgtactcagcaccaagcccactgtccttgagctgggtcacggcccttctttctattttccaattgggggttaagcactgtctatcggtgagagagccgcaggcactgcagggtccgagagaagtacgagggtaggggtgccacaagttcactaggggggtccctggatctggtgcctgggaggaggaggcggtgcaagtgcaggtgcaagggcatctgggccacctgggaaggacgcaggcgaaggcgtctgaggagagcttcgtccagcacctggagtgggaaagacagcagcccacctgagttccagccagaggagcccctggctctgatggacctctctggtctgcaactgccatcattttctcaacaggcaggcagggatttctctccacacagagctaagttacgtttcagctccttttgtgtttagtgaagccatgtgattaagccactcaccaatgggatgtgaggaaaacacagacctagccctcaaagccccgattcacaaaaacatccagtctccccttatggccaggaagcaatagcccttttctccaagtggctacccagagtcaggtccactcctactgtatgcccatttgccagacttaatccaagaagaaacaatggacttcagggcctgtcagaggtcagctccccactccttgagctaacagacagaaggaagcctgaggaagtcacagcaccacagaggcaagggtggccccagaggccaagcctgttcccccaattccctaaatacagagcaggcatgtgggcctgaagacctacctgtctctggagcttggtggtcttggctggccacagaaagcgctggcccagaacggtgcccactcgaggagcataggtgtggtccccaagcactgggcagagctgtagagccatgtgcacctgaagctgactggggaacactgaaagatggggggaggcagttgctcttcttgatcatcaaggactgtccccattcccagagcctgtgcatctatgaggtgacctctgcctccacagagggccatgagcagatcaggacaatgacagctggcaaccttcccagcctgggggcattgaacccaacagaactgaagctcctgggaataagtatgtgggttgggtgctaaactgtgggtgtgcacctaaaactctgcctcctgctgagcccactctcagagtccccagatgctcttttttttttgttttgttttttgttttttcaggcggggtttctctgtggctttggaggctgtcctggaactagctcttgtagaccaggctggtctcgaactcagagagaggtgcctgcctctgcctccccagtgctgggattaaaggcatgccccaccaccgcccggccaccagatgctctttggatgttgcaaaacaacatcttcctcataggcttcatttcccctgcagagtactgttcagccctacctgtcagtggctgcagctggaccagagcacagccacagcctgtggccatcacatggaaatggctgagggtcctcttgacaccttctaggatgtcctttcgagatggggatgtcacggggatggcctaggataccagtcaagaatgacttagcacacacgtaggagcccaattgagccacccctgctgccggctgacttaggacagggcctcagaacaggcagcagcaacttgcctgttagaggacaggggaatagccaccccatgcacatagtgcagactcctctgtaaggcaaaacctgaaaggactcctagtggcttctgactcacaagatcaatgccatccatgcgttccagcttcagggccacgtggatagtcccctcagaaggctcagggatgccatcagtgatgccactgagaaagagaaaggaagtctcagagcagccagctgggaccttccttggccctgggagtcaccccgcatctctcaccagtaggtggctgtgggcctctgtgttctccttgagtgaacgaagaacttctgcaagcggcttgctgtctgggggcagctggagagaagcacaagcccagacgcctccctgaaagaagaggacaagtcacataaagcctcttggttggaaaaaatgagggccagattggctgctcctgcagaggactagcactcggcacccacatagtgggtcccaactgtaattccacttccaagagagctgatgccctcttctggcctctggggcaccgggcatgcactgtggtacacagacacacatgcaggctggcctccgggggcacaaggcatgtgtggtgcacggacatacatgcaggctaaacacacatttaaaaacaaatctttggtcttttttaaaggagaccctcccaccaaggggctggagaaatgaatcagtggttaagagcactagctgctcttccagagtacctgggttcagcacccacatggcagctcacaactgtagctccagttccaggggatctgagaccctcacacagacacacatgcaggtaaaacaccaatgcactatatatatatatatatatagatagatagatagatagatagatagagagagagagagagagaccaccttcccacctccccaaatgaacaccaggactacagtccagacctaagaaacaaatcctggccaggcagtggtggtgcacgcctttaatcccagcacttgggaggcagaggcaggcgcatctctgtgagttcgagaccaacttggtctacaagagctagttccagggcagcctccaaagctacagagaaaccctgtctcaaaaaaaaaaaaaaaaaaaaaaaaaaaaaaaaaaaaaaaaaaaaaaggggggggaaataaacaaatcccaagaccttttttctggcccccagagaactcagacaaatgccctaagagttactacacatgccaggcacctgaataggactcttatatacagtgggtgtaaaataaaggcccaccacactcctcacttactttccaggtgctcgcacaacctggagctcccggtgtttgagccccagggcctggctcagctgtggcagcacagaaagcaaggtcagctccccaggtctccctgagaagtaagagggagaaaacaagttgccagagcaccatccaacgaactcctccccaccagcctttccctccttgtccctgcccatacctgtcacaggcagaccctgtggcttgttcagtgtcaccagaggtcctgaaatattcggtagatgtcagcaaaagcagggcagcctgtgtgcatgcagcctctggttataagctcccaagcccactccaaactgaacttcctgctccccgcacccagggcctttgcacctgctgccctgagctttcattaatattgggtcccttatcacacacctccctataatgtgtgcccctcctacagtccttgactgtttggtctgagaccagctcaaacaggccggcctcaaactcaactataggtaagtctggtcttgaactcttgatcctccttgcctctgtctcccaagttctgggattacaggtgtggtcaccactcccagaaacagcagagattcactattcctcacatcagaacgcttgctctctcaaggcaggagccagccatatttatcccagtactgtgtagcctcctcattgctacaatgcactgaagttcagcaaagttgtcattcctaaaggcacacagcatgctgcttacagaccccaggaaactaacttctgctcccgagggtcttagaatgcgaggcggtgtgaaggtttcctatgcctggcggagggaatgtgacggctacaggggtccgactccagggggtcggaggctccctcaccttgctgatccaccacagctgccctcagcacctcagccagctcctccgggccgaggttctctgttcgcagaagcccggggaagggctggtcctccactgcgtccttgcacttgctggaaccttgaggttgaggccgacccctgagaaaaacgacagaagccggtacctccaacgcccacgctgcccagtgcagccttttcgtacacaaccctcagaacccccggctcggctgacagttgtggtccacaggcccccaagactacagaaggggcatcagacgctcggagcgccaaccatctcgtgtcgggtcacacaacgcggctgggattagaacccatgtcctgatctgagtgcccgacctgccctccccttgtcctagcctccctcctacacggaagtgttgagttataatcaccgggcctcggtgccgaagcctgcatctctcggccgtgcgccagccgccagccggggccgccaggtgccagacacacaacgccaaacgcggaagacgcccatcgccgcagcaagcacagacgcatgcgtgtccacggactgccccaccgcgtgcatcctccgtgcgccgattggtcagcctggctgtcaatcagagcatcgagcaggcggagcttcgaggacggaagagccaaactttcctttggctgaggaaggtaaagtacggtctccggctctgctttgggggaaactgaggcaggaggcgcggttatttactcgcggtagaggacgtgtcttagaatagaaagaggcgagtttgcagatagattgtttcagttctcaggacccttaatctgaaacccatccttgtaaagcagagggagaaggtgaagccccacagccttcgggccagggcacctgcatggaaatggtctgcgacattaaccatcccatgactgtggctcaggtggtagagagatttttctaggatgcacgagttcctgggttcgatccccagcaccacacgaagaccaagatcacagatacagactgagttggagaccagcctgggatatgtaaggcccttctatcacaagaaaaacaaactaggccgggtgtggtggtgctcttcttcaacagaggccaacctggtctagacagtgagtttcaggacagccggagctacacagagaaaccctgtcccccccccccaaaaaaaaaagaaaagaaaaacaagcttggtgcggtccacatagtaagttccacactggccagtatgttgttaattccaaagatggaatgagtgtaaggaaaaaggccagccaaagaaacacactttggctctgcaaccagaaccaaagaaatgcacccttgtcagaagccagccttggtgacgcccccccccccaatgcctagctagccgccagctagcctagcatcccacccctcaggttccaggacctggcctgagaagcaactaacaccttcccccatttccttagttagtgcttgagataaacataggagattccagatacctggcctgagaagcaactaacacctattctttctccacacaggagattccggatacctgcctgagagcaattaacattcattcctcccccacctcctttttctctgcttcctcctttctctataaaaaccccttgtaataatcaataaatgggccttgacaagaaaacctgcttggtcccgctctttctttcccgcccatcattttcaggcgtgatccacctcggactcaggaattaccggagccccgagggccggggtacaccctgaccctgaaaccagtgccagagaaacacactttggccctggaatcagagccagtgaaagaaatacaccctggctctaaaatcacagtcaaaaggctaaaaaggaccagtgaaaggaacacactttggccctgaaaccagagccaaatctaaccctaaacctgtgcagaaaaccaaccaatccctgagcaggagatctagccattctgagctcagggattgaaatctgaccaatccccaccctggaaatctccctgggaaaactctgcccccccctaagaatccctatataaaccctgtgcctgttcagcttcaggctgcctggctgccctctgccactggattcttgaatgaggtttgggtaagaactttcgccggagcagagaccaccctacacactggctctccaggggactggagttgaactgttacaatttaaccagggaaaccctctcctccccaagcagagctgatacactgaggaaggcctttccctgaagcagggctgtaagccgctatgcttactgtgacctgtggcattccttggctcctaaacgccagaatacctttccatcccagctgtaacactcagtattctgtgactccggagtgccagaatactttgcatctgagccataacacagtattctttggctcccgagtaccagaatccattccttcccatccaatccgttgaaatgcttacaatgagaaaaaccacgaacccaaatcattgagaccaaattaattaaagtttttttttcattcatgtacgagactgcctcccccaaggcctgatttgagaggtcagcattggatgtgaggaagacaagggggttttgttgttttggttttttgagacagggtttctctgtgtagctttggagcctgtcctggaacttgccctgtagattaggttggcctagaactctcagagatctgcctgcctctgccacttgagtgctaggattaaaggcatgtgccaccaccgcctggcctcacctggatcacacagacacatatatacataattaataaaatacagacctggcctttaatcccagcactaatctacatagtgagttgcaggacaaccagagttacatgaccctgtctcaaaaaaaaaaaaaaaaaaaaaaaaaaacaacctaaataaataaaatgcagaaagtaacaatatgtacataataaaaataatgaaataataacataattgaggaacagtggggcatggtggcatgtgcctttaatcccatcacttactgatctacatcccaagcctgaaggaccttgctgaaagggctccagcactagactgttgcttctggcagagaaagtggctctcaacctgctggtcaacgacccctttgggggtagaagggccctttcacaggggtagcctaagaccatcagaaaatacagatattcacattacaattcataacagtagcaaaattacagttataaggtagcaacaaaaataattctatggttaggagtgggtctccataacatgaggaactgtattaaaggttgcagcattaggaaggttgagaaccaccaatttaggggcagatgctgcctccttcacagcactcagccacagttgtgttgtgtccttgttgaggttctatccggtcctccaaagttcagctgtgggaaactaaatctttaacttcataataatggtaattggaggctggagtaattagtaccagataaaatcatgaagagtgtgcttccatgatggccattaacagctttctaaggagagcagagaaaaaaaccttttcctcgttgtggaatgacttcctccgtgttggtgtgcaccagaaaggccccctgcttgtgctaagtggagctatgccatgctcttggactctgcagcctctagaaccatgatccagatcaactgtgtgtctgtgtgatttgttttatttttattatgtgtgcatgcctgtgtgtctgcgtaagtgtataccatatgtgttgcaggtgcacggagaggccataaaaggggcatcagatcctccgaagctagagttttaggcagtcgagagcctccacgtgggtgctgggaactgaactcagagcctctgcaagctcagccagtgctcagccagtgctcctcagtttgtgtgcttgttttcagtactgcagatggatccaggaccccacacgtgccaggcaagcattctctcactgagctgcaccctcagactggtctcaggtggtttgttatagcaacagaaaacagacatgtggaaggagtgatggcgtatcttgactgagaggaggaataacttggctactcgcacagcacatgtctgggcaggttcacaaggaggcttccagggacaattggcacctaagaaccctgacctaatgaatggatgaaccattcagaatttggatgggttcttgggggggggtgagggaactgtggtgggtggggactgcttggaggaagtagggtgtttgttgtgggtggctctcggaggctatgtcttacattggctcctttctgttttggttctttcaatttcctgttgtgtgctattctgctacaaccgcctcacccagtagattgcagcctctgttaatcaagacagggtcttactgtgtagccctggctggcctggaactcatagagatctgtttcttctgcctcttgagtgctggaattaaagacgtgtgccaccaccaccacctgacctagaggacaactttattttttttaattttttatttattatgtacacattgttcctgcctgcgcaccagaagagggcaccagatctcattatacatggctgtgagccaccatgtgggtgctggtacttgaactcaggacctctggaagagcagccggtgctcttaacctctgagccatctctccagccccctggatgattaattgtagaagtttcagttctttctttttgtttttgttttttgagacagagtcacactacgtatcttcaactgatctggaacatgctacgtagacaaggctggcttcaaatgtgtgtcaatgttcctgcctctgcttccagagtgctgggattataggcatgtccctaaatctgttatttttttaaacacttatttttgagggggagtcggtggtggtgctgttgtacgtacatatgtagagatcaaagggcatcttgtgggagtctgttctctccttccaccaatgggttctagggactgaattcaggttctggagcttgacagcaagcaccttaatctgctacaacatcttgttggtactaaatccatggagaattgaaaggattgtttattgttgggcccgggaatatagctctgtggtagtagaatgcttgcctaatatgtgtgagattcttaaaaaatcattgattgttttcttaaaccaactttttaatttgttaaaaccaacttttaaaggatatatctaggcatggtggcacacagcattaaccctagcacttgagagatagaggcaggcagatctcctatgaatttgaggccatcctattctacgagttccagtacagccaggactacaggactgtgtgatatgcacaacacaggaaggactgatgacaaatttggtaaaaaaaagaaagaaaaacagcaagtggtttcacattgtccggcttcacctggagtgggtgactgtgggtactcctgaagcctcccaggagtgggtgactattctgtcagactgtgggtattcccgaagcctcctaggaggcctcctgtgcatcacagaagtggcttgagcaagaacttgggacagactgggcaagtgcacagctataatcccagcactgcagaggtgagggctgatgggtcaggagctcacagttatccttgactacaggaagtctgaggccagtctgagctatgtgagactctaaaacatacatgagtcagtgagttggctcagccagtaagggtgcttgataccaagtctgaccacctgagtctgatccccagaatccacatggtaggagcagagaaccaactcccaggagctgtcctttgacctctggacttatgctattgcatatgcataccaatacataactccctaaaatacaaaatacatatgtttgggctttttgtttgttttgttttgttttttcgagacaggtttctctgtgtagctttggagcctatcctggcactcgctctggacaccaggctggcctccaactcacagagatctgcctgcctctgcctcccgagtgctgggattaaaggcgtgtgccaccaacgcccgactgatgtttgggcttttttgatggtttttatgtgtgtgtgtgtgtgtgtgtgtgtgtgtgtgtgtaattagaaggagaaagagagagagggagagagagacagagagacagagacagagacagagacagagacagagacagagagaggagagagagccaaagtctggtttgtggcccattcaactaagcagacttctcctccaccaatggtgtccaccaatggcccaggaaaggccatttccccacttgaaccagcgcctggccctctggagggttagttcctttactctttggaggtctccctccctgccccatagcaggctgccctctcttgctcccttcccacaggcagctaccttcatcccagtgggctatgagctcctggaagaaaaggaggagccaaattttgcctaggctgagggctcagtgccaggtggcagccacagtcaactgctgaacttgtgaaactggaccccaggagaagaagcctgggacaaacatcagcttgcatcagctccctctggctcagttacagctttgcagcaggtgtggacaggctggtgcttcagcaatgggaaacaggactgatgcaaaccaggcctccaggaggccaagccctggagccagcctgcagtgaaccagcctccagccacatctacaactgtgtgagggcctagaatagaaagtgtacttgacctgtctgtccttgagtgctcttctcttctcacacttggctccaggcatggggcagcttccagcgatggctttccagacaagcttgagtaacttggccctttgtcttagtgttttctaaatccaaataggctttacagggaggattgtgaaattatgttagctttggatctgagagccagactgaaacctggcttcctacaggcctagtctttcctgcttaggtcagttacctgtcactaataaatgggggctccttttcctaccagccaccaagatggctgaagtggaggagaagaaatgaaccttccacaaattcacatactacaatgtggacctggatcagctgttgacaggttctaggaactgctgatgcagttgtacagcaccccagaggtggtgtctgaaccacggcctgtggcagaagcagtactcgctactcaaacacctgagaaagctcaagaaggaggcaccaaccatggagtaacccgaggtgctgaagacccacctgagggacacgatcatcctgcctgagatggtgtgtgtgtacaatggcaaaaccttcagccaggtggaaatcaaaccagagctgatcaactgctacctaggcgagctcttcatcacctccaagcccatgaagcatggccagcctggtattggtgccacccactcctccagctgcatccccctcaagtagctgtggccaacaaagactcatgtttaaaaagaaaattggaagccaggcattggtggcacacgcctttaatcccagcactcgggagacagaggcaggtggatctctgtgagttggaggccagcctggtctccagagcgagtgccaggataggctccaaagctacacagagaaaccctgtctcgaaaaaccaaaaataaataaataaataaataaataaaataaagaaaagaaaagaaaagaaaaatgggggctcactctgagaagacctgactcctcctcctcctcagaactctctggtttgtttttgcagtgctggggtcactgtgccaggctagagactcactgcaatctgagggtggaagtgctgagcaggaacagggaactgtgtataagctggcataggcattgataaattcccctgtagatggaccaggacctttcaacccgaacacatggaaagttattgtaaaatacaacagttgggaatcaaaagagtggtttgatccaccttacaggcaaatttcattcggaaactgacaacacatatggatcatgtggtctgcctgaatatcaaatatggcaggcctcaaaaatcaactgcagattttaatataacatttaattattcatctattaattaatttattctgtctcataatatcaaaccttatctattaaaagggagcagggctggggatgtggctcagttggtagagaatttgcctagcatgctggaaaccctgggttccgggttcaatccccagagccacataaattggatgtggtgtttcacatctgtaatgctagcacttaggaagtggaaacaaatggatcataagttcaaggtcatcctccactacataataaatttggagccagcctaggcttctgtatctagaagaaaaaacagggcaccactgatgcaactcattgcataaaagcaattgctgtgtgagcctgacaatccaagctcaatccttagaaccaagagtggaatgaaagttgtcttctgggccatgcacgcctttgatcccagcactcgggaggcagaggcaagtggatctctgtgagttcgaggccagcctggtctacagagtgagttccaggataggctccaaagctacacagagaaaccctgtctcaaaaaaaaaaaaaaaaaaaaccaacaaaaaaaagaaggttgtcctctgacctccacacctgcaccatagtactggcataccaacacacacacacacacacacacacacacacacaccagtaatgacaaatgctatctcctgtaattctcggcaaatagtttcaaacaaagaacacaggcaaagatgaggtattggcataaataatttacttcaggctctaataccatacattagtggggaatgagaacaaaaggaggaattgtctgacttcccctggggatcacaaactctatccattcggcccagcaagggcgccaatgaaaggtgaagaagccacgattccatccaagtccagcttggtgaaccggtgagtttactaggttacttacaggtggggcctgggtgactcaaaatcacaggtgactccctccaaatctgcatcagtgacatcctggctttagttaaccttttacctcttatatactctagcaccgccccaagatcatgagcagctggggcggggcaggagggaggaatggctgggatttcaggtgctaagaccccctgacactctcctccctttctaatacaggtgttaactatttccataccctagccataggcctcaccgtcactgtggcatttggttcattttgttgtcttgattcaggtcaaagtattctggaggccaccatagcaatgtctgttgcttgatgagcatggtcaaggcaggaggggactgcagagagggagtggcacatagggatggcatgtgaagaccgagtggcagacttggtgccaaagcctgccagggacaagtgtttgtcccctaaactacctgtgacatgaaggaaggaactgagccaggctaggaagttctcccgtgaccagcccaccccagagctccagcccttcctgcagtttccttggtgctgtgctgtgagaggggtgcaggttggtgagaagctctccagctgccaggcttgtgggtgcttctagcagagtgcaaggtctccagtcacatccttggctggggacggcatctgaggacttgggccttcattgcagcatcttcagacaggcggggaggagggaggaggtcttggccttggacggctgttcttcctcagtggcatccaggaactgctgcatataactggaggagccaagcagcatgtggcctgtggcctgcatgctgaagagggcccagcggagcatttccctgggagacagcaaggcactcaggttaaacactccccatgctatctccccaaatgtcttctccatttttctatgctggcctaaagcagttggcttcaagttagacttctttctttgtttctacttcttttatttttgagatagaatcttaacactttatctcaggttagctagaaattcatttatgtagctcagtctggctttgaactcacagaaatctcctgcctcaatctcctgatgctgttgcagttatgggccaccacacctaactttttttggtttggtttggttttgatttggctctttttgaaacagaatctggagatcctcctgtcttgtcctcccaagtgctaggtatacagacatgtcccaccatactgggctcaagctagcttttctccttaggaagatttaagtgtcactaaagtaaaatgacaaaaatgcttccaagtacagacaggaactgggaaccaggctcatagctagccatcccttacacatagttctgcctctactcttgtgacaacttttctggaaccccttgctttttcccatgcttaacttgagttctcaaaacaaagctttttattttcttgtaatgatattttgtttctttcttttctcttctatggtgctggggatggaacccagggctttatttgtgcataagcagctgacctgccatggagctatgtccccagccccatatattttttttttcaagacagggtttctctgtgtagctttggagcctatcctggcactcactctggagactaggctggcctcaaactcacagagatccgcctgcctctgcctcccgagtgctgggattaaaggcgtgtgccaccaacgctcggcatccagccccatattttttaaactactctggaccagccagattaatttacataacacatgtctaccctttgtaacactgcttcttagactttatagtcttcccaggccagttctcagaatgctggccacaaggctcccatgcctgatatcatattctaagcatagaacttggaccagacagggctgaacactgatccagagtggtttgtttatatctaaaatatttaaaatatattttaaaatattataaagatgggtgatgtaactaatttagtgcttgcctttcatgaacaaagccctgggtttgatccccagcaccgcaataacccagagtggtaatataggactgtaaacctaggatccagcactgtggaggtagaagcaggtggatcccaagttcaaagtcatccttggctacatagcaagtgtgagaccagcctgagatacatgagaccctgccaaaaaaaaaaaaaaaaagctttaactgttctggttctgagctccagcagccagaagctttgtgacttgtaaaatgggtaatgagtttgaaactaatgtgaagcacttagcatttggtctgatggacagaaaatgggacatgcagccaagagcagctggctgaatgcaacttctcccacagctattgacaagggctcggcaggggtcactaggagttgctggtcttaagaacaataaggaagagaaaacacaagaaagagaaatactgccaggccgcggtggcacatgcctttaatcccagcactcaggaagtagagacaggcaaatctctgtgagttcgaggccagcctagtcttcagagcaagttccaggacagctacggttacatagtgataccctgtctcaaaaaagcatatatatatatatatatatatatatatatatatatatattgaatttagtggcctgaggcaatggctcagtgggtaaagtgcttgccatatggccatgaggccctgaaagcccatgtaaaactggggatggctgcctgtatctgtgaccccagtactcctctcatggtgagacagagagacaagagactcctctgaagctttcaggccagcaagcctggcccagacaacagacaaacagcaaagaccctgcttgaaacacagtggaagatgagaccagcaccttaggctgtcctctgacttcgaaatgcacgctgtggtacatgggtgcccacattcacacacatatagagataaagactggctgggcagtggtgacacacgcctttaatgccagcacccttgaggcagaggcagttagatctctgagtccaaggccagcctggtctacagagtgagtttcaagacagccagggcaacacagagaaataagaaaagagaaaaaaaaaagatataaagactgaaactgaaaatataatatattagggctagaagtatagcttaatggcatggtgcttgtctagcatgtatgaaggtcctggtttaatccccagctttgaagcatgtgtgtgtgtttgtttctttttgtggtactggggaactggacacaaggccttaggcaagtgctctgccattgaactacagcctcagctttcttttcttttttaaaaatgtttatttattttatgtatatgagtactctatctgcatgtatgactttatgctagaagagggcgtgagatcccactatagatggttgtgagctaccatgtgggtgctgggaattgaactcaggacctcaggaacagcagccagtgctcttaaccgttgagccatctctccagccccctcagctttctttttacccccccacacacaatatctctctaagttgcccagtttagccttgaacttactctgtagcctaggcaagcttctaatttgccatcctcctgtctcaatttcctaggcacctgggagtacaaggccatgtatagctttatatatatgtttcgtgcagtgccttcaaagttatttctcaaattagagaactgagttttgactctagccagccagtcttaaaaaggaatgaaaataaggcctattctacaaatgaatgaaccttgaaaacagaattcttgtggggaaagaatcatgatcccatttctatgaggtctctaggatagatcaatggagcaacagaaagtagagtaaaggtgagcagggtctcgggagagggctgagaaccattatttactgagtacagcttctgctgcctggtgcaaaggttctggaaaccaaggcaatgcttgcacaacatgattagttacttcatgctagacacttcaagcggccacagttgaaaaatgttgaatgtgcagattttagcacgggtgttttttcttgaaggtctcattgtgtagctctagctagaatttggaacttgaaatgtagaccaggctagcctaaaattctcaggagatctacctgcctctgcctcctgaatgctggctgggattaagggagtgagctaccacacctgaccccttagtacatttttttttttaaatcacagtaacaccactaagtcactcagctaaccacagtttgacatcagtttttattttctctaagtttttatttttgttgttgttttattttgtttttcaagacagggtttctctgtgtaacagccctagctaccctggaacttggtctgtaaaccagactggccttgaactcacagagactcacctgcctcagcctcccgagtgctgggaccaaagatatgactcctggcctttttattttctttccatgtgtgggaggtaggggttatgcacctgagtgcagtgcccacagtggtcagaagagggcaacatatctcctagagttgcagttacatgtggttgtgagctggctgaggttggtgccgggaactggacctaggtcctcgggctgagtggttccctttttctgtttttgttttcaatacagggtctgatctggcccaggctgtccttgaactcctgattctacacctccaaagtactggaattatagttacattttcacttacaaaaatattagctgtggaaatagctcaggatatgagtatgtgcttagcatgcgtgctgccctgggtcctatccccagcacacaaaagaaagcacatgactgtcattctttgttagcccccagtgccaacccaggctttgagagagttcagctttgggtagacacaaaggacctctggttagctagtctcccaccctgctgtcccaatctactcacttcacgacgcgcttggcccggtagcagtgcaggtcataggaaagtgtgtatgtgtcatagagggtcgccttcacgttcaggcagccgatgaagtcctgggggttcagcttgtataggtcaaaggttacccgggccacatcaatcttctttgttggcttctgggaaagggatagctgtggtctcgtcttgcgctgcaagacaagtccatgttagctcgcctctgggactccttccacgccctcgacactcacccctgctggaggagactgtcacctgttctgatgggggcttccacttctgccccttctgcaggaccatgaacacagtatctcttgccagggcttggaagtattcttctgtctcaacaatcgtgccgtcttcctccagcacgagggagaagggcttgtctttaagtttcaagatatcctgggcctggaaaagaaggttggtgacatttctgccatccctatggagccacagagttgaggagcaaaggcccgcagtaaagcacttcagctgccagagagagagcaagtgagctgccatccatccgtgcgatggactgactgtcactgtgggggcccactcaagggctgtgtctcagtacttcctactcccaagaaggagtgtagtctaatagaatgggagtcatatgtgaggtcttttttattgagaaataaattacataccatgctgggcaatattaggagtcatatcgaaggccttgggcaagctaggcaaactgcagcattgaactatatccccagccttctttgtactttttgtttggagacaggatcttactaagttgtttaggttggcctggaactcactgtgtagtataggtaggcctcagccttctatttagatcttcttgactcagcctcctaagtaacttggattacaggcctacactaccaggcccagctaaactttattttgttggtggtggttttttgtttcttttgctatgtagccgtggctggccttgaactcacagagatccacctgcctctgtctcccaagtgctggggttaaaggcacgttgttatggaataatcttttggtacactgtgaagttgtgtctttgtcaaggcgctttctgactggtttaataaaagaactgactggccagtagctaggcaggaggtataggcaggaaagcaagacacagaggactctgtgaagaagggcagagtcttgggagtcatgagcaaatgcagagggaagcaagatgaaagaaggtaccactatgatgcagagggtagatagtaaaaggattaatttaagtcatatgagctagctaaacacaactctaagctatcagccaagcatttataactaataatgagtctttgtgtagttatttgagaactggctatcgggatagaaaagtctgtctatagtgtgcatcaccatacctggccactattttaaatttttatttttaattatgcgtatatgtatgtatgtgggtcccctggagcaagtgattgtgagctgcccagtgtgagagctgggaactgaacctctgtcctctgaaagtgcttttaacgactgagccaccactctagccccaacaactcatttgtaatcttgttcctgccaaagctacatggcatgaactgaagaaacaggtcattcaaaactgagggacaggaaataaaataactggcttgccatctttaaaagccagctagaagaatcagcaggtaagggtacttgcagtgcaaactttagcatttgagttcaatacctggagcccacagtagaaggagagaaatgactcccaaatgttgccctgtgacctccatgtgtaccctgtggcatgggcatgccagtgctcacacacacgcttcatagacttacagtaataattttaaatataaaataaaactgtctggatttgcaggacaatcagcagtgctccaacagagtctcaagataagcttgggggggttcttgttatgtagccctagttgggcaggaacccctatatagagaccaggctgacctcaaacatgtggtcctcttgcttcttcatgcatcaccacactcagccttagttttaatttcttttgaggcatacccagcgtatcctggaactcaattatgtagcccaggctagcctcaaacctgagatcttccatatctggtctcccagatacgagccaccatgcctggctcatttctccacgtgtaaatatggggaaggggaatgaatggccctttcagcaagaaaataaccacccacccacggttggtgtgaaagacggtagtgcactgccctccttctgtcacttgaccatgtcacttatcacacacacagctctccaggcggaaatgccaaccctccagtgccaacacctttctccgcttaagaccatgccactcatgcgcacagacttcagggctctccagaaaccgaagcagccggcaggctcaactctcagccaaggcctggagagagcctatgcagggtaccggcagatcccttaccttgcccaggaggtcctccaggctgtgagccatgatgcctttgcgaaccttccgatctgccgtgctaactcgacagggcctcgccctggggacttcccggctgggcttagacaccagctgttgggtcaccactgcagtgctcactgctacatgcctggggacaacagtttgtacagcagatgacctgcctgcggctaccaggtctgcccatgcctgcctgcttgctgagttcaggtgtgacctttccttccaaccaatcatggctgcttcaggcggctttcacaaagtccatcagaagacacagctccgatttggaggggcagggaaatcagcgggaatcttgcttaaatcagactcacttcagtgcctctgagtggagcccaaggactgctaacaagtccccaagctgtgtcagtgtggctggttcttggaccagccacattctcctgtggatggcttctgcttttgtggccttgttcaccttgcctaagaggcagggtgaaatggagctggtgtgtgtatatatgtggggtgtgtgtgtgtgtgtgtgtgtgtgtgtgtgtgtgtgtgtgtgtgtgtgtgtgtgtaaatcggcctgtagaatatgtggggactctgaggtacacagggtagaaagaaaggagcagagagaatttgggtcttggctggaattccaagtttctctctgttacggatttgctaggtgacagcatacccagcctgtgactcagtttcctcatctgttgacttggcatagttgccaccacttcccagagaggagtgaggctcctgggagatcaggaacaaaggcacctggtgcccagcctgaccctttctactccagatgtgatgggaactgtggcagtgacagagagtaagatggggactgtctagactgcaagatcttgggtgctaccctaggcctcctgaatcagatctgtgggggtttattgtaagattttgtctgttttgttttaatgggggtcttgcttatttgtttgttttttgggatagcatctccatatatagccctggtttactggaactaatttcctcatctctgtctctgtctctgtgtctgtgtctgtctctctctctctctctggacctggacctatgcagaccagactcaaactcaccaagatcctcatgccactgctccccaagagccaggattcaaaacatatgccaccatgcctggccatatcagctagaccccacatgactttttttgtcttttaaaatgggatgcctatgtagccctgactgacctgggacttgctgtgcccactacaggctggcctcaaactcagatctgcctgcgtctgcctcctgagtgctgggattgaaagcgtgtaccatcacatccagcccctacatggtttttatacctattaaagttttcaaagttctaatagatggctccccagctttccccttccttcccatacccagctctccttacctggacagtgacttagggtacaggaggctgagagacttcatggcatagtccatccttgtcagctggattgtgttggacctggatcggaggcagtaacctatctggttagagtgggaagggagggaggggtccctctctttctttctttgactcagtggatcccagcccccacacggcaaccctctgccccagtctctcctcctacccccaatttccctgaatccacaccaactacactgagtcagcgtctaccatccccacccattccacagccccagaacaactcactccatgtttctctgccccaagctctggttacactgagctgaggaacccaagtcaaggctgaagaggccctcactgatttgtccctgtccctgggggcagagtccacattatgctggcaggaggtgagtcataccacctcatgctgtgagagacttgaaagtcacctattgtccagaaactccacaggcagtcagaccttggagagcctgctctaaccatgggcccttgagcaataaagtcctcctgaggcttagaactggctaagagaactttctgtgatcttaaagactgtctaggcttgtgttcaccaataccttagctgttaccactgagcccttggcaccataactaatgtgacaaaggagctgacaccgaacttctattgaggcttaactaatttagttaggtaactactgtggtttgtgacgccaggtaccccactggacattacagttgtagtggagtaatcctgggctcatgtgccccctgcctttcctcctatattgctgttctctggttcttgcctgctgagctgcaagtctctgcacaggcagtctcttctacctgcaatgcttgtctcaaactcagagctcaacataagtcacctagaagccccagctcctggctgccaactcaggaaagccttttctggtgccctcttgcctggtaccagattgatccctgtgccctgcccttccatgtgaaagacccctctcccttagccctttctttctcaagtttgtctcctcttcctcctgtcggcggcttccccgatccccacccccggtggcctttccccgcccggcccgagaacaagcaccgggtggggccggcccgagaacaagcaccgggtggggccggcccgagaatgagcaccaggtgggccagcccgagaacgagcaccgggtgggctggcacgagggcgagcaccaggtgggtcagcacgaggacaaacaccgagtgagccggcctggagctctgcccctgagcccccgccccgcccgaagagaaacactccgtcccaaggtctccgcccccaaggtcagccatcaggaaaagggggggaattgagtctgctgtaccagacaccagaccttgagaatatgctgatctggaatggctctgtgtctcatttgaaccatccaatggaaatgattctgtatttcgcctcatttgaaagactctgtgtttcacctcatttgaataactctgtactttgcctcatttgaataaccctgtatagcgcctcatatacattgaccaatgggaatagctctgtataatgcctcattagaattatccaatagaatccttgctcctagcttgcgccttttttcctatataaggaccccttttcccttggctcggggcgcttagccacacagaagctaagtcgccccaggtacctgcgtctccaataaagcctcttgtttttacatccagttcgtggcctcgctgattcctgggtgtgtgggtctccctctacgaaagtgcctcttcggggtctttcatttgggggctcgtccgggattgagacccgcccagggaccaccgacccacgtctgggaggtaagtgttgtgcggatccgctgttttgtcttgtctggtctgagtctgtcttgtgaattgcgcttgcgtttgtagtatacagctgtgtacatttgtaggcggatccgaggagggactgacgggtccgaactcccgaccgcggctccaggagacgtcctggtagcgtttgaagccctcaggaagagggatttgtattttgaacttgggaagccctcagggtgagagatttgtactttgaacttagatctatgactggacattttcccagtctctttggagaaggccctcggcttgagggatttgcaatctttactggggacgaggaaggagggcccccttcctcgactctctctcaattccttctgtcgactctctgtcgaaaccgcgctgcgaaagtctgttctgtgttattcggtctttgtcttgtagctgtcatttgtgccctcctaagcctagaaactatggggcaaactgtcaccactcctttgtccctaacactctcccactggaaagatgtacaggaatatgctcataaccaatctgttaatgtgcgtaaacgcaaatggattactctttgttcttcagaatggccgacctttgatgtaggctggccgcgagatggtacctttaacccccagactatattccagataaaagagaagattatggatcctggaccacacgggcatcccgatcaagtggcttatatcgtcacttgggaggctttggttcaggacccccctccctgggtacgtcctttcttacatcccaagggcccctctctccttcccccctctaaccgctccaaccgacccattccttcggcccctacacctcccactcctttgattcctcccaaccccccttcccattccaacctttaccctaccgtgatgaaagacactaaggctaaagaaaagaagacacctaaggtactccctccgggagaagaccagttggttgatctattaacggaggagcccccgccatatccgccactgccgcccccaccagaggcagaagcggactccgccgctgccttggcggaagcggcccctgacccttcaccaatggcttatcgactaagaggtcgtagggagcagcccgttccagattcaaccactctgcccctccgaactgggctgaacggccaacctcagtattggccattctcagcatcggacctctataactggaaaaataataatccttctttttctgcagaccccgtgaggctgacatctctcatagagtcggtactcacgactcaccaacccacctgggatgattgtcagcagcttttgcaggtccttttaacctcggaagagaaacagcgcgtgctactagaagcacgaaaaaatgtcccaggagtaaacgggcagcccacccagctacccaatgaaattgatgcggcttgccctcttgaaagacctgaatgggattttaccaccgaagcaggtaggacccacctgcgtctctatcgccagttgctggtagcgggactccggggggcaggacgcagacccactaatttggcccaggtgaagcagataatacagggtgcggaggaatcacctgccgcttttctagagagattgaaagaagcgtacaggatgtatactccctataatccggaagatccaggtcaggccaccaacgtttctatgtcctttatttggcaatcagccccggacataagaaacaagcttcaaaggctagaaaatctacagggatatacactccaagatttgttgaaggaagcagaacgtatttttaataagagggaaacacagacagaaagagaagaacgttggaggaaggaaactcaggagagagaggaaagactaagacaggaagctgaggaaaaagaggttgcgagagaccgtaagcggaataaagaaatgagcaggttattggccacagtagtgacaggccagagacagaatagacagagggatgacagaagggggccccacctggacagggaccaatgtgcttactgtaaagaaaaaggacattgggcaagagaatgccctaagaacccccgggccaagcttccaccgccaagggtttctgacctcctgaacctagaagattagaggagtcggggccaggagcccccccctgagcccaggataacactgcaagtcggggggcatccggtcaccttcctagtcgatacaggggcacaacattccgttctgaatcggtcacccggacccctgagtcacaggactgcatgggtacagggagctacaggcggaaagcagtaccattggactacaaatcggcagctccagctcgcgaccgataaggttatgcattctttcctccatgtgccagactgcccctaccccttactaggacgggacctattgaccaaattaaaagctcaaatacactttgagaggtcagaagtcaaagtcacagggccagagggaattccccttaccatcttgacaatgtccatagaagatgaatatagactccatgaaaagaggactaattcgaacaatcaggaaacccttgatcactggcttgcggaatttccccaagcctgggctgaaacaggaggaatgggccttgccattaaccaggccccaattatagtaaccttaaaagctgccatccttcctgcatccgtcagacagtatccaatgcctaaagaagcccgcgaaggaattcggccacatattaaaaggttacttgaacaagggattctggtgccctgtaaatctccttggaatacacccttgctacccgttaggaagccgggaactaatgactaccggccagtacaggacctgagagaagtcaataaaaggatagaggacatacaccctactgtccccaacccttacaatttgctgagtggattgccacctaactatacctggtacacagtcttagatcttaaagacgctttcttctgcctccgcctgcatcccaccagccagcctatatttgcctttgaatggcaggacgcggcccttggaatctctgggcagctgacttggactaggctacctcaagggtttaagaacagccctaccctttttgatgaagctttacatcaggacctggcagaattccgggttaggtaccccgctctaatcctcttacaatatgtagatgacattctcctggcagccaaaaccaaagggaaatgcaaggaaggcactcaagccctcctccagactcttgggagcctagggtaccgggcatccgccaagaaggcccagatatgtcagaaacaggtgacctatttaggatacaagataaaggatggacgtcgatggctaacggaagcccgtatgcgagccatcttagacattcccaccccacaaaatccccgccaactgagagaattcttgggaacggcaggcttctgccgcctatggatccctgggtttgccgaaatggcggctcccctctaccccctcactcggccaggggttgcttttaaatgggaagagccccaaaagaaagccttcaccgacatcaaaaaggctctccttgaatcaccagccctgggtctaccggacttagctaagccatttgaactttttatagatgagaaggagggctatgctaagggagtcctcacccaaaatctggggccttggagaaggcccactgcatacctctccaagaaattggatcctgtggcatcgggatggccaccctgccttcgaatgattgctgctatagccctgctggtaaaagattctcacaagctaaccttggggcagcctttgaccatacatgcccctcatgcagtagaggcagtcatcagacagcctccagatagatggcttactaatgcccgaatgactcattaccagactatgctgttagacaaagaccgggtccacttcgggcctttggtgactctgaacccagccaccctgctccccctccctggggagcccgaggctcatgattgcttacaggtattggccgaggcccatggagcgagatccgacctgactgaccagcctctacctagcccggaccacatctggttcacggatggaagcagctttttgcatcaaggagaacgaaaggcgggcgcggcagtcaccacagagaatcaggtcgtctgggcccaggcactcccccctggaacttccgcacagagggcagaactcatagcactcacgcaggctctaaaattggcagaaggtaagaggctcaccgtgtatacagacagtcgttatgcctttgccactgcccatatacatggagaaatttacagacggagggggctgcttacctccgaagggaaagacattaaaaataaggaggaaatcctcgctctcttaagggctcttcatctgcccgctgccttaagtatcatacattgccctggacaccaaaaaggggattctttcgaagcaaggggcaatcgaagggcagacttggctgcccgagaggcggccctgaccacagacaccactaacctcctggctctagagcccaccaacgaccatcccttcccctcatgggactatgaacaaagagacatccaaaccctagagaaattgggagccgcaaaggaaccaaacggggattggacttatgaaggaaagactgtcatcccctaccgggtaaccaagtacctagtgacatttttacataagatgacacatctgagctccaagaagatgcgggagctcctcgaacgagaagaggaattcaatttccttttggggaagaacgatattctaaaacaggtaactgagcaatgtgatgcgtgcgcccgagtcaacgcatccagactgaagcttcctcccgggaaccgggtcagaggctaccggcccggaacacattgggagatagatttcactgagattaaaccaggaaaatatggatacaagtatctattaatttttgtagacaccttttcaggatgggttgaagccttccctactaaacatgaaacagccaagatcgttactaagaaattgcttgaagaaatctttccccgttatgggatgcctcaggtattgggaacagacaatgggcccgccttcgtctcccaggtaagtcagtcagtggccaccttattggggattgattggaaattacattgtgcttatagaccccaaagttcaggacaggtagaaaggatgaatagaacaatcaaggagactttaacaaaattgtcgcttgcaactggcactagagactgggtcctcctactccccctagcactctaccgcgctcgtaatacccctggaccacatgggctcacaccctttgagatcctgtatggagtacctactcctatcattaactttcttgatcaagatgtctcagattttgctaactccccttctctccaagctcatttacaggccctccaactagtacaacgggaggtctggaaaccccttgctcaagcttataaagaccagagggaccatcccaccatcccccattcctaccagatcggggacactgtttgggtccggcgtcaccaggccaagaaccttgaaccccgctggaagggaccctacatcgttttgcttaccactcccaccgcactcaaggtagacggcattgcagcttggatacatgcttcacatgtaaagccagcccaacccaccgattcagccactgcatcagaatggaccgcacaccgcactcaaaatcctttaaagataagactctctcgtacaccctcctgttgattggttgtctgtttaccccccatgtagcaactaacccccacagggtttataatatcacctggaaaatagccaatctagggaccggggaaatagccaacctcagcacttatatagggactctacatgatgggttccctcctctctatgtcgacctatgtgacttagtagggtctgattgggatccctctgaccaggaaccattcccagggtacggatgccaccaccctgggggaaggataggaacaagaagcaaggatttttatgtttgccccggccataaaccaactcatggctgcggggggccgcaggaagggtactgtgcaagatggggatgtgaaaccacaggggaggcttactggaaaccctcttcctcttgggatttcatcactctcaaacggagggagatcccagggtacgcagggaaaggaccatggagatgtgggcaaagagcctgcggaccttgttatgatagtgccggagggggaggttttcaaggcgccacccccggaggaaaatgcaaccctctcatcctaaggttcacagatgctggaaaaagaactacttgggatagtcctaaggtctggggactcaggctgcaccgagcagggaaagatccggtgactttattctccctgtacagacaaattactcccctaagccaacaatcagttgggccaaacatagtaatagcggaccagagatccccaacccattttcaagtccctaaaccccctaccgttcctaaagctatcactcctacaccaggtgctgtcaccttctcccccaccccagatgccctaaacatcgagataaccagagaccctccaggtaccagagatagattattacaattaatccaaggagtttaccaagccttaaatttttcagaccccaacaagactcaggaatgctggttatgcctagtttcccggcccccatattatgaaggcgtggcaatactgggcaactactccaaccagacctcagcacctaccagttgcggagctgctatgcagcacaagctcacaatatctgaggtctcaggaaaggggctatgcataggcaggattccttcctcacatcaagaattatgtaaccaagtagagccattatctcaggacagccgataccttgttgccccttatggaacttattgggcttgcagtactgggttgactccctgtgtctctaccactgttctcaacaccaccattgacttttgtatattgatagaactttggcccaaagtcacataccaccaacctgaatatgtttacagcgtactagagaaatcaacccgatataagagggagccaatatcctttaccgtggccctattattaggaggaataacaatggggggcatagcagccggcatagggaccggaaccgttgccctacagggaattaatcattttaagcttctacaacaagccatgcacacggatatccaggtcctagaagagtcagtcagtgcactcgagaaatccttaacatcactctctgaggtggtcctgcaaaacagacggggattagatttattatttttacaggaaggggggctatgtgctgccctcaaggaagaatgctgcttttatgcagatcatacaggaatagttagggatagcatggccaaacttagggagaggctaaaacagaggcaacagctatttgagtctcaacaaggatggttcgagggatggttcgctaartccccctggttgactacccttatatccacgctcatgggacctctggttattctatttttgatcctcatatttggtccctgcattctgaacaaactgactcaattcatcagagaacgactatctgttgtacaggctttagtcttaactcaacaatatcatcagctaaagcaaatagatccagagtatctagagacctctgaatgaaagattccattcagttacaagagaaatgggggaatgaaagacccctctcccttagccctttctttctcaagtttgtctcctcttcctcctgtcggcggcttccccgatccccacccccggtggcctttccccgcccggcccgagaacaagcaccgggtggggccggcccgagaacaagcaccgggtggggccggcccgagaatgagcaccaggtgggccagcccgagaacgagcaccgggtgggctggcacgagggcgagcaccaggtgggtcagcacgaggacaaacaccgagtgagccggcctggagctctgcccctgagcccccgccccgcccgaagagaaacactccgtcccaaggtctccgcccccaaggtcagccatcaggaaaagggggggaattgagtctgctgtaccagacaccagaccttgagaatatgctgatctggaatggctctgtgtctcatttgaaccatccaatggaaatgattctgtatttcgcctcatttgaaagactctgtgtttcacctcatttgaataactctgtactttgcctcatttgaataaccctgtatagcgcctcatatacattgaccaatgggaatagctctgtataatgcctcattagaattatccaatagaatccttgctcctagcttgcgccttttttcctatataaggaccccttttcccttggctcggggcgcttagccacacagaagctaagtcgccccaggtacctgcgtctccaataaagcctcttgtttttacatccagttcgtggcctcgctgattcctgggtgtgtgggtctccctctacgaaagtgcctcttcggggtctttcacatgttcatttctgctgcagctctcgggtccccactgtgcctgttttcagcccgtagcacagggacccagtgtgaacaccaggagtgggcaggcatggagaactgcctcctcagtgaaggaaaaccattcttcccttctgataactcgggttctccccggtcaaccttacagagttagagaccctcgtccccacttaatgctacccaagcactcttgtttcaccctgtttctaatacccaccactcacacagctccacaaaacccaccacacctgctctgtggtagccaaataccccagctcttctcctcccctcacctcctagcctcctggcaatactgcgtggccaggcacagcccaggcctcctccattttatttctatctcctgtgaagccactcccttggccccagccccagctggggaaaagagcacaggagtgagtctgaaactctccctagggatttcaagccagactgacttccagaaagccccgaaaggggaagaaggcatgtttcgaaagctctagaaggaagaggtactgcccaggacttggtaccaggcaagagggcacagaaagcaacagacggggcttgtggtctgcgttccacccctcacaccagctgtttgcgctcacccgagttgccttctttcagccctggattttctcctgtgcatgaggaataacgctcctgttgaagccaggaatggtggctcagagctgtaatctcagcacttggtagactgaggcataaaaagggccatgagtttgaagctagcctgggctatagagttaggtcctgtctcaaaaacaaaaatatagtcagacggtggtggcacacacttttgatcccagcactcggaaggcagaggcaggcggatctctgtgagttcaaggccagcctggtctccagagcgagtgccaggataggctccaaagctacacagagaaaccctgtctcgaaaaaacaaaataaataaataaataaaaaagaaatatctttttttttttgagtcaggcaagatacttgagttttattatgtctgtctaaaagcaggtgttaggcctatttgtagtgaagcgtggtgtgctggcgctgcaacaccggtgaggaagcaggaacctgatctcagagcccaagaactgcttgacagcaggccttcggcacttgccctctgaaatctcttccaccttcatcaggttaatggagtgtgcacgggcacagtgcgggcacccgtgtcttggtagcactgtttgacggctccagcagcggtcaggtcccggtattcttggcacatgttgtgtgtgtcatgttgtgagttgtagtgcagccataccgcaaagttcttcacccgcagtggggcctcaggtcgcactgcacagtgtacgatctcctcggatgacttcttcatcttgttcatctgtgcccaaaatcgggacttggccaccacatggttcgtcgcaaagatgtgcatgaggtataggagtggtgtgtggcacttccgggtgggcaagcagcgccccaccaccttgtactcccgaagcgtgcctgaggccttcgtggctgatctgtccttgttggccgccagccacaaaaataaaaggaaatatcttgcctataatgttacaatcccatccagcacttgggaagccaaggcaggaaggcctctgtggatgagaccagcctgggctacagagtgacaacctgccaagagaatggggtgggtaatcccaaatcttgggtatagtgaagtacaggactcaagagacagaggcaggaggatcatctccagttcaagacaagcccgagctataaggcctcaaaaacaaaaaaagaatattagatctatgatttattgctataaacactacaacttaaaaaaaattaaaataaggacagcaagatgactcagtgaatgagggtttgaatttgatccccagtacccacatagtacaaagagagaactgactcccataggttgtctttccgtgaaaactctccctcccttcctccctctctctttctctctctctaaatgaaaatgtttacatcaaagcctctacaaagagcattcatgttacacctagcgttggagagatgtctcagtggtcaagagcgctgacttctcttccagaggatccaggttctattacaagtacccatgtggcaattcacaatcatttataactcagttccaggggatctgactccctcttcaggcctctgtgggccttcatgtgtgtggtgcaaagacttgcacgaaggcaaaacactgtacacataaaataaaaataagagacagaaaagggaatttattcagtgtggccataccagaaaagatgagaggaccagtctcttcaaccctgtttttggagtgcagatggtagctctaggaatttacaggagaagaacaaaaaatgggatgagctggtggtgcaggcctctcagtgcttgagaggcagaggcagaggcaggggaatctctgtgggtacaaggacagcctgactgacctggtggattccgggctaaccgaaactacaaaataagacactacctctaaagttaaaacaaaaaagggaggcaataagtatgcatagctaagcattctattctattctgcccaaaccactctaattgacggctacctccagtctgaagtaagggtcctgcagtagctaggaagaggactacctcccaggctgactgttcctggctctggcaacaaactgaaaaggaaaactggttcagaaggagatcagagcagaggggccaaattagaacgaggattgtgccttccttcaggtttagaacaaccactggagaattttaggtactgctgtggtagtttggaacaagacgaggcaaaaacttattactggcaatctgtaatgtccccataaatcctttttatatctatttttaaaaagatttctatattatatgtacagcattctgtctgcatatatgcctgcaggccagaagagagctccagattttattacagatcgttgtgagccaccatatggttgctgggaattaaactcaggacctctggaagagcagccagtgctcttaacctctgagccatctctccagccctctattttattttttaaaagaattattttatgtgtatgaatatttgcatgtatgtatgtgcaccacattcatgcagtgcctatggaagccagaagaggacgacagattccctgaaaccagagttacaggggctgtgagttgctttatggtgctgagaactgagcttccttcctgtacaagaaaagcacatgctgttaaccactgagctgtccctccagccctccccacccctttgtgtgtgtgtgtacttgtatgtgtatgtatgtggacatgtgagcatgtgtggaagtcagaggatggtttgcaggagttggttctctccttctaccctgtgggtcctagggataaaactcaggctggtagcatgtccctctacctgcaaagccatctcctgggctttgtcccaataaatttcttgctctcccttggtatcttcattcttaaagggaaggacagagctacaggtacaatgggtgaaaattggcctttagttaccagtcttgaagatgagtacttaaatttttttcagaatcctttttttttttttaaatctttttatttatgtatacagtattttgtctgcatgtatgcctccaggccagaagagggcaccagatctcataatggatggttgtgagccaccatgtggttgttgggaattgaactcaggacctctggaagagcagacagtgctcttaacctctgagccatctctccagctcaagaatcctttttttttttttttttttttaaggcagggtttctttgtgtagccttagttgttctggaattaggtttgtagaccaggctggcctgaattcaaagacccacctgactctgcctcccagggtgctgggactaaaggtgtgtgccacgacacctggctaagatgagtgctttttaaatgcctatcacatctacaggcacaactgattgggaagctgattcaaaccttgagatagcaaagggatagaggtgttgatgcaacataaagacagagatgcccagattttctccttcctttagtcactgggcgggactctgcaggtccagtgaggactcactggctctcttgttaaagccttatttagagttaagccaatccttggaagaggcatcattcattcattgaacacttgcagtgtgttaccctgttaattcagcagaaagcaagacagctttaggccacacctggagcagctggaccagagaccaagtggaagcaggaatgctgatgtacacttctcatcctgatcccctggaggctgtcagcctgggccacacagtgcgagatcctgcctcaaaaacgtttttgctacccattggtgattgtacatgtactttgttctagcactcaggaggtagggccagcctggtctacaaaggacagccaatgctacacagagaaaccctgtctagaaaaacaaaacaacaacagaaacttttttgtctgatgtttcatcttgtttcttttctaataatgtactgggcagagaaaaacaaaataatgcagttgggcctggaggcttagacctgggattccagcacttaagagatagaggcaagctcattgctttgagtttaaggtccctctgccctgcatagtgagttgcaggtcagtgtggactatagagtaagaccttgactttcaaaaagcacaaggggccaggcgttggtggctcacagctttaatcccagcactcgggaggcagaggcaggaggatctctgtgagtttgaggccagcctggtctacagagggagttccaggacagcctccaaaacaatacagagaaaccctgtctcaaaaacaaaacaaaacaaaacaaaacaaaaatgtcaaaaagcacaagggccatcaaaatggctcagtgggcaattacaaacttgatgacctgagtccattccttgagacctacataatggaaagaactgactcccctaagttgtcttctgaccttcacacataccgtgtgtgtgtgtgtgtgtgtgtgtgtgtgtgtgtgtgtgtgtgtgtgtacatgcacgcgctctcgtgagcaagtacgctcctgatactaaattaataaaaaggattaaaaaagtaaaaccacaaacatgaaccaaaccaatgtggtgtaataacctatgtctggagaactaacaggtttctggacccagagaggtagagaagataatctaatgtcttcaggggaattagggggataaagcatcagagcataaagaaagtttccctcacttgggaagtagtggaggcaggagcatcaggaattcaaggtcatctcatctatatagtaagtaagttcaagactagcctgggttacatgagaccctgtctcaaaaaaaaaaaaaaaaaaaaaaaaaaaaaaaaaaaaaaggaagcatggtgcaacagatttgagattgaaacacaaaaatagctcaactctggggcagacagcagagtcaggggaaagtctaaggaagttctggggaaagggtcaggaggcccctccctgcttcattcacgccccaaacatctattaaacattccatgtgtacttggtgtttctggcctgaggatgactcacagaggctgacacactaagttaatcacaggaagcacagaagaaaaaaaaaaaaaaaagaaaaaacaggcctaatgcaatggaatgcctgactgagggggttcttcacagtgctttggagacgaccctttgtgtctcattgaaattcatctctcaagaaacactcctcaaatgtttacacattgtcaggcactttgagctgcagggacacagaggactcagttctggtccctgcctttggaattcacaattgttgctcttccaaagaactggggttcggctctcagcacccacttaaggaggcccatgaccatttgtaacttcagctcaaccgcttccacaagtacccacacacatgtctgtcccccaaacacacctatattcttcatgctaaccttgtgacactatgagcaaaatcaaagacattaagaagccaggttcagtgccctgtgccttagtcctaacatttgagaggctaaatcaaacaggaattgaggccacttggacagcatcaagagaaattatctttgaacatatgaaatgatattatcacagaaatagaattgcaatttaaaccaggtatgtgatcccctctccccccaccaaaagaaaaaaaaatagagggctggagaaatggctcagtggtaaagagcacctgctgttcttgcaaaggatctagattgttttgttttgttgagacagagccacactatgtaactctggctgtcctggaattcactatgtagatcaggctagctggtatgtatgtctgtccacaaatgcatgcctggtgttagaggcttccagaagtgtgtgtcagaccctctggaactggagttataggtggttgtgagctgccatgtgggtgatgtaattgtacttgggccctttgcaagagcaaccagtgcccttaacccctgatacttgcttttaaggaatttgcatttttgttttgaaattacatgttggaaaagttttccacatcagtaagaaatgccatccttactatttcttcctgcagcttctcagaaatattttttaatctttttttttaagattttatttatttatttattatgtacacaacattctgcttcatgtatatctgcacaccagaagagggcaccagatctcattcaaggtggttgtgagccactatgtggttgctgggaattgaactcaggacctctagaagagtagtcagtgctcttaacctctgagctatctctccagcccctcagaaatattttaattcaaaaatctttcttagccagctctcaagccactggattacttcttgcttctgctactgacatagacttcatcttgattcactccatacagcaactagatgtgtgtatataaatgacagatagatcatagatggatggatggatggatagatagatagatagatgatagatagatagataatctcacttgctacctaggctgacctcaaactcatgcctcagtttcctaagtgatgtgattacgggcatacactattatgcctagcataaccatttgtttgccttaaaattttttaagattaattttttattatgtatacagtattgtgcctgcaggccagaagagggaatcagatctcattacagatggttgtcagccaccatgtggttgctggaacttgaactcaggacctctggaagaacagccagtgcttttagcctctgagccatctctccagcccccagcctttaaaatttttaattaattttttttttgtttgtgtatgggtgtttggcctgcatgtaggcctatgcactatatgtgtgtagtactcacctagaccagaagagggtgccagagactctggaattggagtttcagtgcatgatgagctgccaagtgggccttaatccccaaaccccagagggttgaagagggatcagtggctaagggtgcttgttcttgcagaggacccagttttgattcccagagcccacatggtagctcagaacaagaactccggcttcagaggattctgcaccctctctgggcctcatctggtaccaggcatacatctggtatgcagatatatacacgccctgagtggtgacaaacactctgtgagttcaaggctggtctacatagagaattccaggacatagtgagaccctgtctcaaccaaacaacctggatcctttgcaagaagagcaggtgcttttaaacactgggccatctctccagcaccacctcccccctttaatgatacataggtcccacatagcctagttcggcctcttaactcctgacataacacctcatgaattcctcatcctcctgcctctaccttttgagtgagtgacgagattacaaacgttcgccaccatctttgttcccagttggccctcaaggccccagggtctcagttcagatcctgccactatggatacatagcattgaaatggggaaggctgcctctgtgctgcctcagcctttgggtgacgcctcttgcttggcactgcattttcaggagcatgacatctttgctctggaccctgaccccaaggttgtgtggagctgtaagaagtgtaagggtgtctggtattggtggcgcgcgcctttaatcccagcactcgggaggcagaggcagcagaggatctctgtgagttcaaggccagactagactacagagcgagttcaggacagccagggctgttaacacagagaaacactgtcttgaaaagcaaaacaaaataaaaccaacaaaacaacaacaacaacaaagtgttaagcgcgcatgcaaccagccactgggcaattagtcaaagatgccaagtctagatagacatgcagttaagaacttagggtctagagagatggctcggtggttaagagaccagaagagtttgcatcccagcgctcgcacagtagctaacaacagtctatcttacaccctcttccagcctctcctggcacaaggaacacacgtggtgctcatagttacaaaacagacacaacacgcatacacaaagaaataactaatttttaaaatgtcttgtaatcaagtaaaagtgctaactctagggactaaattaattcctcagtcgccctactccaggagcggtaaggctggccagaaagaccaagaacgcacgcgcggaggagaaaccacagagtcggtcctcccgggatagagaggccggaagtgctcgcggagctgcacgccgggtgctggaagcctactgagccccgaggaagggctccgctcggggcttggcgtggtgggtgagccggagggtcggcgtgagcggcctgggctttggttctgaatgatggcgtctcgggcaggcccgcgagcggccggcaccgacggcagcgactttcagcaccgggagcgcgtcgccatgcactaccagatgaggtatgaggtgagccaggagcactgaggccttccccgggaggagcctgcgggtctcgggaagcgacgcgggcgagcctcacggtgccgctcccccagccagctgtcgcgtactaccgggtccccggctccggcgagcgcctcgggtctgtttacaggccgggaagcccagtggcctgccctcgcccgcctcgtgctttgaggggatctggcctgcagaggctcaggggtcgatgctcagcccctctgaatgaccttggagacatcatttttcttttttcaaatcgaggtcccgcagtgtctagagttcaggctggcctggaactcacggccttccctcctcagcctcccgagtatgcgcctggtctgaaaactaacattcttaaagaaaactgcgtgtgtgcgtgtgtgtgtgtgtgtgtgtgtgtgtgtgcgcgcgcgcgcgcgcgtgtgtgtgtttgtgtgtgtgtaagtttgcccacacgagagcaggtgtgctggcagaatgcagtgccagtaaagggtgtgtgtgtgtgtgtgtgtgtgtgtgtgtgtgtgtgtgtgtgtgtgtgtgtgtgtgtgtgtgtgtgtgtgtgtgtgtgcgcgcgcgcgtgtgtgtgtttgtgtgtaagtttgcccacattcttaaagaaaactgcgtgtgtatgtgtgtgtgtgtgtgtgtgtgtgtgtgtgtgtgtgtgtttgtgtgtgtgtaagtttgcccacacgagagcaggtgtgctggcagaatgcagtgccagtaaagggtgttggatctcttggagctatagttacagacgtgtttaaatccacctgagactagtactgaggaccaaaccccgaagtgattttgtgagatagcggcttggtatggggtccatgctagcctccaggagcggcaggtgctcttaaccactgagccatctctccagtccccaggctgtcttggaacttgctctgtagaccagactagaaatcagagatctgcctgcctctgctgccaagtgcaaggattaaaggtgtacactacagccgccctgttgaccctgtctttaggaagatgtcatgtgtactttacaaagagttttgatgtagtcatgtcttaagctttgttgtcacacaagatgaaattaattaccaccagggaatttttcatcaaattgtgctgtacttaaatatgcctaaagtaaaatacttggggtcagactcttgaagtttaaaccaacaccaggtactgagttgtgttgaacccacacaactaccttgcctcctgcagattgttaactctgctggctgtggtggttgaagagacccccacattggcttagctgttaagagcacttgctgttcttggagagggtctaaactcagttcccagcaccgacatcaggaggctcacaaccaaacacctgcaactccggttccaggggttccgatgccctctggcttcccgacagcacaggcgtgtgcgtggtgcacacacatacacatgagataaatttttaaaaagaaagaaaagccaatctgggtcctgccttcaaggagcttgagtgccagaagtgattcttttctccttacctcccacatttcctataacaaattaattttccccttaaatccactgaattattcaactggttcttggcctctttctttacagaaggatgacaagccctttggacccttctttttcaactttaggcccctgcctaatggatgctcttgttttcatgcgtacctagagcagacaccttgcttgtgacaatatgaaaaggatataattgagaccccgagaagatcaggactaagagatgtgcctccttctcatggaaactatatctggagaaatacagggagcccaaactgtgttccttgttgctaggcctccccctaatggcctttgtttctgtgatacgaagccatctttctaaccacagctcagaattcttgtcatggcttggtgagataatgtcttagtacgttgtccaggccaacctccaatcagagatcctctgcctcagcctcccaggtgccattactgttcttcagtcttgaagtctacacatgcatggtctttgtaactgttactacctctctgtgccttggcagttgatttcctgaaacacaaatactaaaaagcttattcctttgtgcctgcatgtttttgcctctgcgtagaagcccacttccactcgtctcattacctctgctgaaatagctctctcctctagatataaccttctctggctttccatctcatgctaattggttcttccctgggatagcggatatcttcggtttatatgtatttacttgagttttatacttcatgtttcccagcctctatcagaggctgtgtcattcagattgtatctatgtgtccagtaccggtgcctggtggtcggagcaatttaaatgaaaattgcttctccctctgctctgccttacagtgtgacgctcaagagtgaaatcaagaagctgatctacgtacatctggtcatatggctgctgttggttgccaagatgtgtgtgggacacctgaggctcttgtcacatgaccaagtggctatgccctatcagtggaatatccatatttattgagcattgtgccctctctcttgggccttctctccttcctcgaaacaacattagctacctggtgctctccatgatcagcatggggctcttctccatcgctcccctcatttatggcagcatggagatgttccctgccgcccagcaactctaccgccatggcaaggcctaccgcttcctgtttggtttctccgctgtctccgtcatgtacctagtgttgatactggcagtccaagttcatgcctggcaactgtactacagtaagaaactcttagactcttggttcaccagcacacaggagaagaaacgtaaatgaagcctgcctgatggacacatgaagggagctgttcagaatctccatggactgtggcatctgtgatgttggcacctagtgcacactatcctcagattttggccttgagttctctgttaccatctgctgagatgacaaatctgtagtgtttaatttattcttgactagccacaaaccggatgaccgatgtctgtggaacacttcaagttgaggccctccagactgagccttatccttgccttctcttggtcaaattccttacttccatttatcacctcttcatcaccgatactaaaagagatctggaataaatcagtgcagaaattctacttcaatctgtaggtcatggggcaagcaacatttgggaagttgctcccctaaaggctactctgtttactgcaaaccattttaattaaaaaagaacttcaataaagttaagactgtcctgtgctttgtgtttgagatttgattgaatttcaaagttgtattgctctagaggacttagacattatgagaagaatagatgtttcctgagaacatgggactggcgctggaggagagacagtgtgaagagtcttaagtcagctgcagccctctagctcttaggtgagaacaaccctgggatgggggttgctggaggaatggctcaggggttaagagcactgacagaggacacaggtttagttctcagcacccaaatggtgacttaaaaccatttgtaactactgtcccagggcatttgacgccttcttgcctccggatgcactaggcagacatgaggtacaaaaacaaacatgcaagcaaaacactcaaaaaattaaaaaaaaaaaacatggtctctcaatgtagctgtggatgtactagaactcactaagtagagcaatctgggctcaaactcacagagatcctcctgcctttgcctaccaagtacagggatttaaggtatgtgccaccacacttagcaatatatatttggccagacatttaatctccacccataaagcagcttatttggcttggattatgagctcccctttttatgcttgtttgagacagggcttctctatagctctggctgtcctggaacttgcttcgtggaccagactggcatcaaactcaaatccacctgcctctgcctcccacgtgctggcattaaaggtgtgtgcatcactgcccagctgggttacgagctttctatttggttatttatgtttattagacttttatgtgtctaaaactgagtaataatacccaaaaagaatctgcttgttggggctataattttccagtaactgacaaataaacctgagtcaaaataagaagaaaggggtttgagttctttttcccttttagtttctgtcattgattattccttggcctgcttgtttggggaatgtgcaaaggaagctgctaactctatggaagccagaaaaagaaaaaggtgagattccagtgtcgccactaggggcagactgccagtgacgtaacttcttcactcagcccctcccaaagcttcctccacctccaaaaatgtcaaggactggtaactagtacatgggcctttgagaaaagatagcagggagcttccagtcttgggggaatgtgcagtgaacataacatctaattgtgtgagagacccacagctgagatacagtttaagagtcacacctagaagtcttcacagtaaataaggcagcaaaggtgtgcatccctttgcactggactttattatgcctagatgatgctcagatctgtaactgcagagatctggccactacagttgtaattcttttgtcctggaaatgtgttgcttgtacagtctgcaaagctaattcctccagctatcccactgatcatttctttggagcaggggcagtttccagacagaatttctctgtgtagtcctcgctatcctggaactcagagatctgcctgctgtgtgctgggactaaaggcatgtaccaccatgcccagctcctactgatcatttcaaagataacttcttgtgcctcaggcctattgctgttcctgcttcctgtgtttacgggaacacagctctggatcatccaccctgcaagtttaagtccgtccccactccatatacacactgcctattcggtgaacctcctatagataccctgataagttccctagcttctcagtttaacacttagaactctccctacttaagggaggcctctcagtggtgaagtgctcctctagcatgcaccgaggcctgggtgtgactccctcgtaacaacaaataaaaacaaaccttttctgctcttacattcctttattaatctttaagatgccacttgtatgaaaattcctcaattttcaattcctcaatttgaagaacaactttaatttgtctggttgctttcaccttattcctttattattattttctaacaagacagagcccagaatgaccttaagctatgtggctgaggatgactttgaactcctgatgctttgtctctacttcctaagtgctaggattacaagtatgcaccatcactcctggcttatattactcgtcccccacccccaccccatagaccaagccttgacctttatttttaaaagctgcatatttattctgctttctgacttagtgtttgtgccttcaacactttcacaacccttttctctcctcaataaggaaagcctgcttgatcctgtcacggacacacttggcacacaaggagccaccacagcccctgctgacgtgcttctttgtctagacagtctcataaggactttgggtctcacagcatgaacccttcaagtctgcctgggcacacaccacatggggattcaggtgctttcccagtcttcttggtataaaggtaaacaatcctgttgccagggttcgagacagcctagtttcgttagaggctgtattgtaggaaagcctacaatggtatgtcaaacactggaccattcgagtgcctctaagcaccatccctggaagaggaagagctgtattatttgttttatattgacagaattgctactttgtatgtcttctgtagctcattagtgttcactctgagcttattgaatgaattaacttgcccttttgaggacaaaggtctgtatttcacagaaaacagaactggactgaagcagaaagaagcccatggacaagggagtttgtcatgagcttagctgaggcacacattgctaacttcaaacaactgtgatgcaaacaattaaaactgcagtgcagacccccctgctttctcccaataaattttccacataagttctgtcccccccccaaaaaaaaaacctgttagcacattattagtcaaagagctaaaacctgtttttggtcatcagaatagtgttttactgaaagtctttgaataccttgtagactaatttcactcattgtgaaattagtcaacagttttaaaacactatcatgccaacatcagtttttgttagttttgtgcttggaattctgcttttctagccttttcctataacttttttctgcctcatggttatggtgttcacaagtcctcttgatttagtgggaagaatgctatttgggttagaaagttcagctgggtatgggggtacatgtctaatcccaacctttggcaggttgaggcagcaggaatactctgagtttgaggccagcctgacatacatacatacatacatacatacatacatacatacatacatacaaacactatctcaaaaaacaaagttggagttaaagtatcaaccacaggactaagatcaacctgcctgttcacaggttgctttaaagctccaatagccggttggagagatggctcagaggttaagagcactggctgctcttccagaggtcctgagttcaattcccagcaaccacatagtggctcacagccatccattatgaggtcaggtgccctcttctggtgtgcagatgtacatggaagcagaatgttgttacataataaatacataaaatcttaaaaaaaaaaaactccaatagccaaagtattatgaaggcattctaaaatttttaatttattttcattttatttttggagacacagtttctctgtgtaaaaaccccaactgtcctggaactcactctagaccagactggcctccaactcacagagataaacctacctctgcctcccaagtactgggattaggccgggcagtggtatcgcccacctttaatcccaacactcgagaggcagaggttggtggatctctgtgagtttgagaccagcctggtctgcaagagctagttctaggacagcctccaaagccacagagaaaccctgtcttgaaaaaaacaaaaaacaaacaaaaagcaagttaatctggaattaaaagtgtatgccaccacacctgatgtaaatttaaaattttttttaaattaatttttctatacacaggtgttttgccagcatgtatgtttgtatagcacatgtgaacctggtgcctaaaaagccaaaagagtgcatctgatcccctgggactggagttacaaatggagtgctgccatgtgggtgctgagaattaaaaccaaatcttctagaagagcagccagtgagtgcccttaactgctgcagcatctttctggctcttgtgaaggcattttatggagtctgtaacaccatatgggtatcaaaagccgtgtggtcacctgatctttgcagcaagacaggagagagaaggtgtttgagagacttgagggatggtctagacaccaaagaaaggtttgtgtgttgtgagcaatgactgattattaggttgagattctagccagggagtttactatctcttgtttgatttattacattttattgccatcctaggaattaaatagggttttacatttgctaagcaagtgttctctcaacatccttatatcccctccccgatttctgacacttactatagctcagcttgtccttgaactcagcaatcctgcctccatagtgccgggattatggatgtttccactcccagctgagcctgttcttcatgtgttgcatgggcattgcagatctctcccattgctgtgaggctgaaataaatgtggatgaactttgtagtacatagtcgtttacatatcaagatgcctttccttggtaaacagtgttatgcttgcctctcaaattgggagtgtcttcctgttttaagaataatcctcttccctcttttcttcctctctcctcctttcccctccttctgacagggtctctttatagaatattcctgcctctctctgccttccaaatgttgtgtaggtgtacatcaccatgcctgcctagttagaatgacgtctcagaatgctggaggttaatttcacattcttagtccacattatcctgactggtatgttcaaggtgccctgacagtaggtacagacccagcagacctcaggaagtgaccattacagcaagatcctgtagctgctactcatagcctgttgggagcccgtgcataggaaagaatggcaagaaaaaatggctgccagagggcgtccccatagtatactctgtcactaagcatgcatgcttcagagcttgccaaaacctcctgagtccctggcttggtccccaaataaaggtcattagagcatgaagtcttggtcaactgattgagactcctttggagagtgcaaggcttctatgtagatgcccctgttgcctcctattctgtctaatttcttactctccctgttgacagctgccaatctcctttctcctaggactgtgcccactcatcactcactgtgataagacccctttccttttctttgttccttcctggtctgcctgcctgcccccccccccacctctcccttttcatggtctcatgaaaactgctctcagactcactatgtagctaaggatgactggagctcccaatcctcctgcccctacttccctaattctgggattacaagtttatgccaccacattccactttctgatttccttccttacatcttaattccactagctttatcacactgctaaagctaaaactttctttgcaaaatgagatccagaaacccacagactctccatagtaggttctgacagggaaacagctgacattgtatggtgttctgccttcccatttctttgctgtgtgtgtttttggacaagtctcggtcttcctgtatttccttccttccttccttccttccttccttctttttccattttttatttgaattataaacacgattgttttacatgttaatcccagttccctctccctcccctcctccctaccaccatccccaactaaaaccctacctatcacatatcctttctgctccccagggagggtgaggccttccataggggtcctcagggtccgtcatatcctttgggatagggcctaggcccacctccgtgtatcttggctcagggagtatccctctatgtggaatgggctcccaaagtccacacctatgctaaggataagtactgctctactacaagaggctccatggatttctgaggtctcctcactaacacccacattcaggggtctggatcagttccatgctggtttcccagctatcagtctggggaccaagagctccctgttgttcaggtcagctgtttctgtgggtttcaccagcctggtctggacccctttgctcttcattcatccttctctgcaactgtattccagttcagtttagtgtttagctgtgggtgtctgcttctacttcttccagctgctggatgaaggctataggatggcatataagtcagtcatcaatgtcattatcaggggagggcatttaaagcagcctctcctctgttgcttagattgttagttggtgtcatctttgtagctctccaggcatttccctagtgcctgatttctctgtaaacctaaaattttccctctattatggtatctcttatcttgttttcttctattcttcccccaactcaacctttctgctccctcatatactcatcttcccttctcattctcctagctccttcctccccttcccaatttgctcaggagatctggtccctttccccttctccaggggaccatgtatgtctctcttagagtcctccttgttacctagcttctctggctttttttttttttttaagatttatttattatgtgtacagtgttctgcctgcaggtcagaagagggcaccagatcccattacagatggttgtgagccaccatgtggttgctgggaattgaactccggacctttggaagagcagtcagtgctcttaaccgctgagccatctctccagccctttctaaaactggacattagcctagcctcaagggttgtgatgctaacaatacaagctaaagagagcaatgggcatgatccaaagccagatagttcagcaaatattgatttcatcccttcccttttcggaacagactccagccacaccaaatatgtaaaccagcaagacaaaaaacagcaggctatcttttcccactttttgctttgtctatgtttgttggacaatgtcaaactatgtagcccagaccccttctgtaactttccatgtagaccaggctggcctccaactcaaattcccagcaatctacctgtctctgcctctggagttctgggaataaaggtgtgcatcaccatgcctggcctcaataagtccttgtcttatggtcttcttttctcctcctttacttcttatcctcctccctctctcttccctttccctttatctttatttctttattgtcaccgtttcttttctgcttctcttaccatgctttaacaaccatcaggaagcaactaaccaaatgttctcatcttgagagtcaggtcagaagatctaggagtcaagaaagacacgtgtaaaatagagctcacataccaagcattagaaaactcgcagtggcactaaaagatggctcaggggataagaacacttacatggtgactcacaaccatttgtaattccagttgcagacaattcaatatctccttctgatctctaagggcacccagaatgtacatacatacattcaggtaaaacgttcatatacattaaaaataataagccttaagaattagaaaagagggctggagagatggctcagaggttaagagcaccgactgctcttccagaggtcctgagttcaattcccagcaaccacatggtggctcacaaccatctgtaatgagatctggtgccctcttctggtgtgcagatatacatggaagctgaatgttgtatacata

SEQ ID NO: 2 - DNA, 60000 bp, Cricetulus griseus
  misc_feature (1)..(59559): Locus of ERV insertion, allele 2 (without the ERV-C 109F integrated in allele 1, at position 30020)
tggttctatcgagacagctacatccgcttctccacacagcccttctccctgaagaacctggacaagtgagctttccctccacctcccttgactgcccaggctagtgaggaggtcagcagacaaacagaacagtgggctctgcgggcagggaaggcaggcttttcgccgccactcatcctctgccctgaaggagcttgccaagagggtgcccttgggttaccgaaatgagcaaaacaagaactcctgctaatcaagggcttacatcctagcaatgagcttgagtaatcatggctaacatgctagaaggtgatatgtgttaggaagaaaatgtagatctgggtaatggcagattaagagaaacttgagaggggaaaggcaacttgcagtttgctcttgggatgagaggaaaccttgaggagacctgggttcaaattctatctctgcagcatccagattgtgttcctctagagacatgaaaagagacttaagcctcagtttcagttagagcccagtggtcccatctgtaatcccagcacttgggagtctgagtctgaaggattttgaggtcattctgggctatataatgagacccccactcaagacaaaacaagccaggtgtggtagctcctaactgtaatcctagaacttggctaaggcaggagccaaaatcaagccaggtacggtattggaggcccgtgatcccagtgcttagaaggtagaggcaggacaatcagttcactgtcaccctcagctacatgccaagttttatctaacctgggcaacatgagaccatttcatagaaaaagaccagcctccgtttccacatctgaagactggagcagcaatacactgtagctgcttatcatcaagtacttcacatgtgataagttgttcctgcttggttataccacagccacttctcaatggctcacaatgaagtgagttattgccattttaaaaagagatgagaggggcaaaggttaaatgacttgcctgagatctcatagttgatcatcggtgaagctgggatttgaccatccagggtcatcgtccccctgccacacataggaatggtgaactgaaactaggcccaaagcacttgatacttactgtggcctgcaggcaacactggagcattctgagaaatgactagccctggccgtgggtgaatagggagtggggcagggagtgggggtggggtggaggggtgcagcttctgtgcttctgtcctgttgatcttccagcaggggcccctctcagttgagatcccctagacggctgaccacagggttctgcctgcagccctgaggttgggctccagggctgagcctgtgtctctgatccactctagctctgtgcacctatgtaacaattccatccagagatacctggagacctcctgtcaccggcaccggatgctgccctcggacaacatgtggtccagccagaggtttcaggcccacctgcaggaaatgggtgccccaaatgcctggtctagtgtcattgtacccggcatgaaggctgctgtgatccatgccctgcagacctcccaagacactgtgcagtgccgaaaggccagctttgagctctatggggccgactttgtgtttggggaagacttccggccctggttgattgagatcaatgccagccccaccatggcaccttccacagctgtaactgcccgcctgtgtgctggtgtgcaagcagataccctccgtgtggtcattgaccggcgactggaccgtacctgtgacacgggagcctttgagctcatctataagcaggtgagatgtcccagcacctcccacaggcaaccctacagcaaagccctggctggggtctgctgtgagacagagttcaagactgactctacacacggggcatcttaacacagacacgtcccactggcctgtctcctcatctgtggaagatactgtcctttgagagccattaatgccatgagtttgtagtcataggtagtattttcagaggccctgggacctgctcagtttccatggtaattccaagcctctaggtagtaaccttattctctcatctgtaaagtaagactgtacctggctcctcctttgtgtgctgtgagaatgggtggtttgatcttcacacaagggtttgctaaagtaccaagctggaggacatagaggatatgaggccatgaacctcaaggcctgcctatcaagagttaattattagtgtctatcattgttataacgattattatgttactcatcttcttccaaaaacagatttgatcttgcttatttataataagtatagaattgcttggggtttttgtttttgtttttgtgggttttgttgagatagggtttttctgtatagctttggatcctgttctggaacttgctctgtagaccaggctggccttgaactcacctttgactgcctctgcctcccgagtgctgggactaaaggtgtgcaccaccattgcccgacctagaattgttttttattcagccaaaaaacattaccttgccctcgtggtctaattctctctagaaacacccttaggagcacaccaggggcatgccctatagaaatcctaggttttctcagtccagtctagttgacaactgatattagccaccacagctggacatggggctccaccttccacctcccagtatttggaaggctgagacaggggatcacttttagttcaagggaagcttgggttacacagtgagttccaggctagcctgggcttcacagtgagaccctacttaaaaacatcatacaaacaaacaaacaaacaaacctttaaacgtatgtttttaagggtttctgaattctgaggacagtttttaagatctttgagctaagattcaccaaggtctaggtttgctacaggaagggaaaacactgatcacctgacctgggtggcctcatgtttcctacaggtcctgatcaccttagaataatgtgctggcaaagggttccgtctttgttagggatgccatcactaggtgtccagggtggaatccaggcctgcgtttttagcatgtgcagttctactgagctacacctccagcctaaaaaatgtaaaggaggagatgcatgcaggtgtgcatacacctgtaatccctgggcaacagaagcaagaggactgtcacaggttcaccaccacctggttacagggtttataataaggtcctgtcacaaacaatgtagtcttggaagagaggagaggaaatggggaagaggaagtaatttgcagtttaaaatagagcaaaattgccgggcgttggtggtgcacacctttaatcccagcactcgggaggcagaggcaggcgcatctctgtgaattcgagactagcatggtctacaagagctagtttcaggacagcctccagagctacagagacaccctgtctcaaaaaaaaaaaaaaaaaaaaaaaaaaagcgaaatttgtatggacatggcaacacgtgcctgtaatcccaacacttagggggctgaggttggaggatcaggagttcaaagttatccttggctgtatgtgagcttgaagccaacctgggcctacagcactgtctgttccacaatctgtctttatctgtttgtctccttatgttgttcagctcggtctgttctctgaatgtttgtctcagaacaaacaaaattgaatagggctgggcatacgcacttttcaaatgtcctttgtcccccaggtatctttcattgctgatttggttttgagcttgagggtcagtgtacaacaaggtggttacaatggtggctttgtctgtctctgatctcctttatctaggacagtgccactgctgtatccctggcaccctggtcctttgcaggccaggcaggccagcctgcaccaccgccccacactgttttatctgtttttctccttatgttgttcagatcggtctgttctctgaatgccctgtaaactagaagataggctaacaagctgatggggttcagggttgagatttttggcaaaaaacactcatgtgatgctaggtacctcatgtgactgtcacaaggcacaaccaggaatctctagttcccaatgccgaatttgaccttagactaaggtggctgccaccagagccacatcctcccctttgtagcttttttcatttttctttacattatttattgtgtttgagtttgtacatgtgtgtggtcacacatgccacagcacacatgtgggagtcagagggcaacttatgggagttggttctctcctcccatcccatgggtcctggggcttgaactcagcaagtaccttataagctatctcaatactgtttgcctccaaaatgtattactttgtgtagctgtcccttttctgttgctaataacagaatactacagacagtgtaagttacaaagaaaataggctcaattgtatccatcttttgctgcccatggcatgaaaacacttgcatcccaacacaggccagaggtcgctttagtgtgaagcaggattgagtgcatgtctgcatggctaagactctctacttctctggtttcctgatccctagggctctgggactctagatcctctggaccccttgataatagagggagcttcctgtttctcagaagtgccttttgaccatatatcccagaaactgattccatccatctctgccgtgtgtcactagtcactagatggcgtcactgtcatctctcactagtgtctgagatggccttgtcttctctcctgcccacagcctgctgtggaggtgccccagtacgtggggatccggctcatggtggagggctctaccatcaagaagcccatagcagcttgtcatcggcggacagcggtccgctcatcactccctcatctgctggcccagcaaggctgtggggaaggcaaggactcaggaccccctatccacaggtcagcttctaggaaagatgctggggccaggagcctgggacacactgagaagccagactctgcggccaccacctcagtccccggaaaggggaagaaaggcaaggcaaaaagtgccacagccctggtctgcatcaccctgcagaaatgggagtcccacaacaccagggtgggccccaccttcaacaggttaatgtgtctgaaacagcctgaggcctggggtagtaccatgtcccccaaaccccgcagtgttcccaaggccatttctgcctgctctccaagccctccccaagcatctgggcttgccctcctgccaaaaggccaccagtgatagcaagtatgaaccaaatatctttaaatacataaccaaatgagtattacaaagtagtcaccctgccaggcagttagaccaaaggctcggtcctagagtgcgcgcccagagtccagacccatgctgctgctctagccagcctttgccctcacctttctctggagaaaggtgctgccaccatgcccttccccattcctaaccagccccctcagccctcataacgccctagtgaggtaggtgctattgtccccattttccagccgaggtagcagcaagtttaaggacgttgcccgaggttgcacagctcagaaggggcagagctgggatgcagacccaggtctgttggtctcccaaccctgtgttcttcccactgcctctggaggaggagctgggaggggctccatctgcccttaccttgtatcccccacctttacacatgtactgtggaacaattggtcaggctggggcctcacccagatcctcacagcttcccttctcccacagccccgtcctacctccctagtctccattccaaggcctggctgccttcttcccatgtgctccgaccccagggccgggtcctcagactaccgaatggccaactggtgggctctaaggctctgtcaaccacaggcaaggccttgatgactctacctactgccaaggttctgatgtccttcccacctcaccctgatctcaagctggcacccagcatgctgaagccaggaaaggtgggcctcgagctgtgcctcacaccctggcgggtagtgctgagcagtgggatcggggctgaagggcacgaacagagggcagcgctcggaccatacagcgccccagggaagggcttgtcttctccagaaccctgttccaagacagaggcctgatcatatctctttccctcccctccttgcaccgaggctgctattcccctgcaccttcgaggcccccactttggaagtgcctcgaggcctctgccctttgaagttggacctcttcctagcacccacaggaaagtcacggccaaaggcaagttcaaggccatactctgcgacaaagccagggctgaggcataccccaagaagaggctgagcctccccaaacccttgacccttattctgacatgccggacactgagaaccatgggggataggaggctagagaaacccctgctctgatctctactgccccatcctggatccagcatcaaattaaaaaaagcaattaaagttctctggacttggcttgaataatgtgcggctaggctcataaaagagttgaccagcagggcctccatcagcaagggccacagtccccacccagcgacagacattggctttctctgcagggagacggatgggtggggaaagagccttcactatacagatgatgacactgagacatggcttgcctgagaccacagcaggcggaaagtcagccatcaggagtgccccttcccaaagacaagctgggctggcagaagagcttctgatggtcacagaacattatgacaagagacgggctttccctaaacctcagcctttctccggagagtatgtccctcagtgaggggtcggtcaagacacctcaaactgcaaaacccaagaaacaggtcaagtgtggcatttcctacctgtagagcctcagctgctggtcctccaaaagcggtatctagctgtagcgtgtgaatgcttcctgagtggggcagggtggagaggagagtgcggtgttgaaatccaatgacctgtgtctcccaaagtcagaagagctcatgcctgcacagtggtgtgtgtctgtcctcccaggacttgggaggcagaggcaggtgcatctctgaattccagtccagccagggatataccaagaggcccagcctcaaaagcaaacaaacctcctcgtgaccaggagtacagcagatgccacctccagacctggccactgttcacttgagcagagagcacagtccctggtcaacacttgctttctcagcagatcctcaaaagcagacttgtgaggtaggacttttattattttaagtccagagagcggtcacctgcccaacgccacacagtaagtgagtggtttaactgggatttatcttaggttggtaggactaaggagtcaaagaccttgaccgtactcagcaccaagcccactgtccttgagctgggtcacggcccttctttctattttccaattgggggttaagcactgtctatcggtgagagagccgcaggcactgcagggtccgagagaagtacgagggtaggggtgccacaagttcactaggggggtccctggatctggtgcctgggaggaggaggcggtgcaagtgcaggtgcaagggcatctgggccacctgggaaggacgcaggcgaaggcgtctgaggagagcttcgtccagcacctggagtgggaaagacagcagcccacctgagttccagccagaggagcccctggctctgatggacctctctggtctgcaactgccatcattttctcaacaggcaggcagggatttctctccacacagagctaagttacgtttcagctccttttgtgtttagtgaagccatgtgattaagccactcaccaatgggatgtgaggaaaacacagacctagccctcaaagccccgattcacaaaaacatccagtctccccttatggccaggaagcaatagcccttttctccaagtggctacccagagtcaggtccactcctactgtatgcccatttgccagacttaatccaagaagaaacaatggacttcagggcctgtcagaggtcagctccccactccttgagctaacagacagaaggaagcctgaggaagtcacagcaccacagaggcaagggtggccccagaggccaagcctgttcccccaattccctaaatacagagcaggcatgtgggcctgaagacctacctgtctctggagcttggtggtcttggctggccacagaaagcgctggcccagaacggtgcccactcgaggagcataggtgtggtccccaagcactgggcagagctgtagagccatgtgcacctgaagctgactggggaacactgaaagatggggggaggcagttgctcttcttgatcatcaaggactgtccccattcccagagcctgtgcatctatgaggtgacctctgcctccacagagggccatgagcagatcaggacaatgacagctggcaaccttcccagcctgggggcattgaacccaacagaactgaagctcctgggaataagtatgtgggttgggtgctaaactgtgggtgtgcacctaaaactctgcctcctgctgagcccactctcagagtccccagatgctcttttttttttgttttgttttttgttttttcaggcggggtttctctgtggctttggaggctgtcctggaactagctcttgtagaccaggctggtctcgaactcagagagaggtgcctgcctctgcctccccagtgctgggattaaaggcatgccccaccaccgcccggccaccagatgctctttggatgttgcaaaacaacatcttcctcataggcttcatttcccctgcagagtactgttcagccctacctgtcagtggctgcagctggaccagagcacagccacagcctgtggccatcacatggaaatggctgagggtcctcttgacaccttctaggatgtcctttcgagatggggatgtcacggggatggcctaggataccagtcaagaatgacttagcacacacgtaggagcccaattgagccacccctgctgccggctgacttaggacagggcctcagaacaggcagcagcaacttgcctgttagaggacaggggaatagccaccccatgcacatagtgcagactcctctgtaaggcaaaacctgaaaggactcctagtggcttctgactcacaagatcaatgccatccatgcgttccagcttcagggccacgtggatagtcccctcagaaggctcagggatgccatcagtgatgccactgagaaagagaaaggaagtctcagagcagccagctgggaccttccttggccctgggagtcaccccgcatctctcaccagtaggtggctgtgggcctctgtgttctccttgagtgaacgaagaacttctgcaagcggcttgctgtctgggggcagctggagagaagcacaagcccagacgcctccctgaaagaagaggacaagtcacataaagcctcttggttggaaaaaatgagggccagattggctgctcctgcagaggactagcactcggcacccacatagtgggtcccaactgtaattccacttccaagagagctgatgccctcttctggcctctggggcaccgggcatgcactgtggtacacagacacacatgcaggctggcctccgggggcacaaggcatgtgtggtgcacggacatacatgcaggctaaacacacatttaaaaacaaatctttggtcttttttaaaggagaccctcccaccaaggggctggagaaatgaatcagtggttaagagcactagctgctcttccagagtacctgggttcagcacccacatggcagctcacaactgtagctccagttccaggggatctgagaccctcacacagacacacatgcaggtaaaacaccaatgcactatatatatatatatatatagatagatagatagatagatagatagagagagagagagagagaccaccttcccacctccccaaatgaacaccaggactacagtccagacctaagaaacaaatcctggccaggcagtggtggtgcacgcctttaatcccagcacttgggaggcagaggcaggcgcatctctgtgagttcgagaccaacttggtctacaagagctagttccagggcagcctccaaagctacagagaaaccctgtctcaaaaaaaaaaaaaaaaaaaaaaaaaaaaaaaaaaaaaaaaaaaaaaggggggggaaataaacaaatcccaagaccttttttctggcccccagagaactcagacaaatgccctaagagttactacacatgccaggcacctgaataggactcttatatacagtgggtgtaaaataaaggcccaccacactcctcacttactttccaggtgctcgcacaacctggagctcccggtgtttgagccccagggcctggctcagctgtggcagcacagaaagcaaggtcagctccccaggtctccctgagaagtaagagggagaaaacaagttgccagagcaccatccaacgaactcctccccaccagcctttccctccttgtccctgcccatacctgtcacaggcagaccctgtggcttgttcagtgtcaccagaggtcctgaaatattcggtagatgtcagcaaaagcagggcagcctgtgtgcatgcagcctctggttataagctcccaagcccactccaaactgaacttcctgctccccgcacccagggcctttgcacctgctgccctgagctttcattaatattgggtcccttatcacacacctccctataatgtgtgcccctcctacagtccttgactgtttggtctgagaccagctcaaacaggccggcctcaaactcaactataggtaagtctggtcttgaactcttgatcctccttgcctctgtctcccaagttctgggattacaggtgtggtcaccactcccagaaacagcagagattcactattcctcacatcagaacgcttgctctctcaaggcaggagccagccatatttatcccagtactgtgtagcctcctcattgctacaatgcactgaagttcagcaaagttgtcattcctaaaggcacacagcatgctgcttacagaccccaggaaactaacttctgctcccgagggtcttagaatgcgaggcggtgtgaaggtttcctatgcctggcggagggaatgtgacggctacaggggtccgactccagggggtcggaggctccctcaccttgctgatccaccacagctgccctcagcacctcagccagctcctccgggccgaggttctctgttcgcagaagcccggggaagggctggtcctccactgcgtccttgcacttgctggaaccttgaggttgaggccgacccctgagaaaaacgacagaagccggtacctccaacgcccacgctgcccagtgcagccttttcgtacacaaccctcagaacccccggctcggctgacagttgtggtccacaggcccccaagactacagaaggggcatcagacgctcggagcgccaaccatctcgtgtcgggtcacacaacgcggctgggattagaacccatgtcctgatctgagtgcccgacctgccctccccttgtcctagcctccctcctacacggaagtgttgagttataatcaccgggcctcggtgccgaagcctgcatctctcggccgtgcgccagccgccagccggggccgccaggtgccagacacacaacgccaaacgcggaagacgcccatcgccgcagcaagcacagacgcatgcgtgtccacggactgccccaccgcgtgcatcctccgtgcgccgattggtcagcctggctgtcaatcagagcatcgagcaggcggagcttcgaggacggaagagccaaactttcctttggctgaggaaggtaaagtacggtctccggctctgctttgggggaaactgaggcaggaggcgcggttatttactcgcggtagaggacgtgtcttagaatagaaagaggcgagtttgcagatagattgtttcagttctcaggacccttaatctgaaacccatccttgtaaagcagagggagaaggtgaagccccacagccttcgggccagggcacctgcatggaaatggtctgcgacattaaccatcccatgactgtggctcaggtggtagagagatttttctaggatgcacgagttcctgggttcgatccccagcaccacacgaagaccaagatcacagatacagactgagttggagaccagcctgggatatgtaaggcccttctatcacaagaaaaacaaactaggccgggtgtggtggtgctcttcttcaacagaggccaacctggtctagacagtgagtttcaggacagccggagctacacagagaaaccctgtcccccccccccaaaaaaaaaagaaaagaaaaacaagcttggtgcggtccacatagtaagttccacactggccagtatgttgttaattccaaagatggaatgagtgtaaggaaaaaggccagccaaagaaacacactttggctctgcaaccagaaccaaagaaatgcacccttgtcagaagccagccttggtgacgcccccccccccaatgcctagctagccgccagctagcctagcatcccacccctcaggttccaggacctggcctgagaagcaactaacaccttcccccatttccttagttagtgcttgagataaacataggagattccagatacctggcctgagaagcaactaacacctattctttctccacacaggagattccggatacctgcctgagagcaattaacattcattcctcccccacctcctttttctctgcttcctcctttctctataaaaaccccttgtaataatcaataaatgggccttgacaagaaaacctgcttggtcccgctctttctttcccgcccatcattttcaggcgtgatccacctcggactcaggaattaccggagccccgagggccggggtacaccctgaccctgaaaccagtgccagagaaacacactttggccctggaatcagagccagtgaaagaaatacaccctggctctaaaatcacagtcaaaaggctaaaaaggaccagtgaaaggaacacactttggccctgaaaccagagccaaatctaaccctaaacctgtgcagaaaaccaaccaatccctgagcaggagatctagccattctgagctcagggattgaaatctgaccaatccccaccctggaaatctccctgggaaaactctgcccccccctaagaatccctatataaaccctgtgcctgttcagcttcaggctgcctggctgccctctgccactggattcttgaatgaggtttgggtaagaactttcgccggagcagagaccaccctacacactggctctccaggggactggagttgaactgttacaatttaaccagggaaaccctctcctccccaagcagagctgatacactgaggaaggcctttccctgaagcagggctgtaagccgctatgcttactgtgacctgtggcattccttggctcctaaacgccagaatacctttccatcccagctgtaacactcagtattctgtgactccggagtgccagaatactttgcatctgagccataacacagtattctttggctcccgagtaccagaatccattccttcccatccaatccgttgaaatgcttacaatgagaaaaaccacgaacccaaatcattgagaccaaattaattaaagtttttttttcattcatgtacgagactgcctcccccaaggcctgatttgagaggtcagcattggatgtgaggaagacaagggggttttgttgttttggttttttgagacagggtttctctgtgtagctttggagcctgtcctggaacttgccctgtagattaggttggcctagaactctcagagatctgcctgcctctgccacttgagtgctaggattaaaggcatgtgccaccaccgcctggcctcacctggatcacacagacacatatatacataattaataaaatacagacctggcctttaatcccagcactaatctacatagtgagttgcaggacaaccagagttacatgaccctgtctcaaaaaaaaaaaaaaaaaaaaaaaaaaacaacctaaataaataaaatgcagaaagtaacaatatgtacataataaaaataatgaaataataacataattgaggaacagtggggcatggtggcatgtgcctttaatcccatcacttactgatctacatcccaagcctgaaggaccttgctgaaagggctccagcactagactgttgcttctggcagagaaagtggctctcaacctgctggtcaacgacccctttgggggtagaagggccctttcacaggggtagcctaagaccatcagaaaatacagatattcacattacaattcataacagtagcaaaattacagttataaggtagcaacaaaaataattctatggttaggagtgggtctccataacatgaggaactgtattaaaggttgcagcattaggaaggttgagaaccaccaatttaggggcagatgctgcctccttcacagcactcagccacagttgtgttgtgtccttgttgaggttctatccggtcctccaaagttcagctgtgggaaactaaatctttaacttcataataatggtaattggaggctggagtaattagtaccagataaaatcatgaagagtgtgcttccatgatggccattaacagctttctaaggagagcagagaaaaaaaccttttcctcgttgtggaatgacttcctccgtgttggtgtgcaccagaaaggccccctgcttgtgctaagtggagctatgccatgctcttggactctgcagcctctagaaccatgatccagatcaactgtgtgtctgtgtgatttgttttatttttattatgtgtgcatgcctgtgtgtctgcgtaagtgtataccatatgtgttgcaggtgcacggagaggccataaaaggggcatcagatcctccgaagctagagttttaggcagtcgagagcctccacgtgggtgctgggaactgaactcagagcctctgcaagctcagccagtgctcagccagtgctcctcagtttgtgtgcttgttttcagtactgcagatggatccaggaccccacacgtgccaggcaagcattctctcactgagctgcaccctcagactggtctcaggtggtttgttatagcaacagaaaacagacatgtggaaggagtgatggcgtatcttgactgagaggaggaataacttggctactcgcacagcacatgtctgggcaggttcacaaggaggcttccagggacaattggcacctaagaaccctgacctaatgaatggatgaaccattcagaatttggatgggttcttgggggggggtgagggaactgtggtgggtggggactgcttggaggaagtagggtgtttgttgtgggtggctctcggaggctatgtcttacattggctcctttctgttttggttctttcaatttcctgttgtgtgctattctgctacaaccgcctcacccagtagattgcagcctctgttaatcaagacagggtcttactgtgtagccctggctggcctggaactcatagagatctgtttcttctgcctcttgagtgctggaattaaagacgtgtgccaccaccaccacctgacctagaggacaactttattttttttaattttttatttattatgtacacattgttcctgcctgcgcaccagaagagggcaccagatctcattatacatggctgtgagccaccatgtgggtgctggtacttgaactcaggacctctggaagagcagccggtgctcttaacctctgagccatctctccagccccctggatgattaattgtagaagtttcagttctttctttttgtttttgttttttgagacagagtcacactacgtatcttcaactgatctggaacatgctacgtagacaaggctggcttcaaatgtgtgtcaatgttcctgcctctgcttccagagtgctgggattataggcatgtccctaaatctgttatttttttaaacacttatttttgagggggagtcggtggtggtgctgttgtacgtacatatgtagagatcaaagggcatcttgtgggagtctgttctctccttccaccaatgggttctagggactgaattcaggttctggagcttgacagcaagcaccttaatctgctacaacatcttgttggtactaaatccatggagaattgaaaggattgtttattgttgggcccgggaatatagctctgtggtagtagaatgcttgcctaatatgtgtgagattcttaaaaaatcattgattgttttcttaaaccaactttttaatttgttaaaaccaacttttaaaggatatatctaggcatggtggcacacagcattaaccctagcacttgagagatagaggcaggcagatctcctatgaatttgaggccatcctattctacgagttccagtacagccaggactacaggactgtgtgatatgcacaacacaggaaggactgatgacaaatttggtaaaaaaaagaaagaaaaacagcaagtggtttcacattgtccggcttcacctggagtgggtgactgtgggtactcctgaagcctcccaggagtgggtgactattctgtcagactgtgggtattcccgaagcctcctaggaggcctcctgtgcatcacagaagtggcttgagcaagaacttgggacagactgggcaagtgcacagctataatcccagcactgcagaggtgagggctgatgggtcaggagctcacagttatccttgactacaggaagtctgaggccagtctgagctatgtgagactctaaaacatacatgagtcagtgagttggctcagccagtaagggtgcttgataccaagtctgaccacctgagtctgatccccagaatccacatggtaggagcagagaaccaactcccaggagctgtcctttgacctctggacttatgctattgcatatgcataccaatacataactccctaaaatacaaaatacatatgtttgggctttttgtttgttttgttttgttttttcgagacaggtttctctgtgtagctttggagcctatcctggcactcgctctggacaccaggctggcctccaactcacagagatctgcctgcctctgcctcccgagtgctgggattaaaggcgtgtgccaccaacgcccgactgatgtttgggcttttttgatggtttttatgtgtgtgtgtgtgtgtgtgtgtgtgtgtgtgtgtaattagaaggagaaagagagagagggagagagagacagagagacagagacagagacagagacagagacagagacagagagaggagagagagccaaagtctggtttgtggcccattcaactaagcagacttctcctccaccaatggtgtccaccaatggcccaggaaaggccatttccccacttgaaccagcgcctggccctctggagggttagttcctttactctttggaggtctccctccctgccccatagcaggctgccctctcttgctcccttcccacaggcagctaccttcatcccagtgggctatgagctcctggaagaaaaggaggagccaaattttgcctaggctgagggctcagtgccaggtggcagccacagtcaactgctgaacttgtgaaactggaccccaggagaagaagcctgggacaaacatcagcttgcatcagctccctctggctcagttacagctttgcagcaggtgtggacaggctggtgcttcagcaatgggaaacaggactgatgcaaaccaggcctccaggaggccaagccctggagccagcctgcagtgaaccagcctccagccacatctacaactgtgtgagggcctagaatagaaagtgtacttgacctgtctgtccttgagtgctcttctcttctcacacttggctccaggcatggggcagcttccagcgatggctttccagacaagcttgagtaacttggccctttgtcttagtgttttctaaatccaaataggctttacagggaggattgtgaaattatgttagctttggatctgagagccagactgaaacctggcttcctacaggcctagtctttcctgcttaggtcagttacctgtcactaataaatgggggctccttttcctaccagccaccaagatggctgaagtggaggagaagaaatgaaccttccacaaattcacatactacaatgtggacctggatcagctgttgacaggttctaggaactgctgatgcagttgtacagcaccccagaggtggtgtctgaaccacggcctgtggcagaagcagtactcgctactcaaacacctgagaaagctcaagaaggaggcaccaaccatggagtaacccgaggtgctgaagacccacctgagggacacgatcatcctgcctgagatggtgtgtgtgtacaatggcaaaaccttcagccaggtggaaatcaaaccagagctgatcaactgctacctaggcgagctcttcatcacctccaagcccatgaagcatggccagcctggtattggtgccacccactcctccagctgcatccccctcaagtagctgtggccaacaaagactcatgtttaaaaagaaaattggaagccaggcattggtggcacacgcctttaatcccagcactcgggagacagaggcaggtggatctctgtgagttggaggccagcctggtctccagagcgagtgccaggataggctccaaagctacacagagaaaccctgtctcgaaaaaccaaaaataaataaataaataaataaataaaataaagaaaagaaaagaaaagaaaaatgggggctcactctgagaagacctgactcctcctcctcctcagaactctctggtttgtttttgcagtgctggggtcactgtgccaggctagagactcactgcaatctgagggtggaagtgctgagcaggaacagggaactgtgtataagctggcataggcattgataaattcccctgtagatggaccaggacctttcaacccgaacacatggaaagttattgtaaaatacaacagttgggaatcaaaagagtggtttgatccaccttacaggcaaatttcattcggaaactgacaacacatatggatcatgtggtctgcctgaatatcaaatatggcaggcctcaaaaatcaactgcagattttaatataacatttaattattcatctattaattaatttattctgtctcataatatcaaaccttatctattaaaagggagcagggctggggatgtggctcagttggtagagaatttgcctagcatgctggaaaccctgggttccgggttcaatccccagagccacataaattggatgtggtgtttcacatctgtaatgctagcacttaggaagtggaaacaaatggatcataagttcaaggtcatcctccactacataataaatttggagccagcctaggcttctgtatctagaagaaaaaacagggcaccactgatgcaactcattgcataaaagcaattgctgtgtgagcctgacaatccaagctcaatccttagaaccaagagtggaatgaaagttgtcttctgggccatgcacgcctttgatcccagcactcgggaggcagaggcaagtggatctctgtgagttcgaggccagcctggtctacagagtgagttccaggataggctccaaagctacacagagaaaccctgtctcaaaaaaaaaaaaaaaaaaaaccaacaaaaaaaagaaggttgtcctctgacctccacacctgcaccatagtactggcataccaacacacacacacacacacacacacacacacacaccagtaatgacaaatgctatctcctgtaattctcggcaaatagtttcaaacaaagaacacaggcaaagatgaggtattggcataaataatttacttcaggctctaataccatacattagtggggaatgagaacaaaaggaggaattgtctgacttcccctggggatcacaaactctatccattcggcccagcaagggcgccaatgaaaggtgaagaagccacgattccatccaagtccagcttggtgaaccggtgagtttactaggttacttacaggtggggcctgggtgactcaaaatcacaggtgactccctccaaatctgcatcagtgacatcctggctttagttaaccttttacctcttatatactctagcaccgccccaagatcatgagcagctggggcggggcaggagggaggaatggctgggatttcaggtgctaagaccccctgacactctcctccctttctaatacaggtgttaactatttccataccctagccataggcctcaccgtcactgtggcatttggttcattttgttgtcttgattcaggtcaaagtattctggaggccaccatagcaatgtctgttgcttgatgagcatggtcaaggcaggaggggactgcagagagggagtggcacatagggatggcatgtgaagaccgagtggcagacttggtgccaaagcctgccagggacaagtgtttgtcccctaaactacctgtgacatgaaggaaggaactgagccaggctaggaagttctcccgtgaccagcccaccccagagctccagcccttcctgcagtttccttggtgctgtgctgtgagaggggtgcaggttggtgagaagctctccagctgccaggcttgtgggtgcttctagcagagtgcaaggtctccagtcacatccttggctggggacggcatctgaggacttgggccttcattgcagcatcttcagacaggcggggaggagggaggaggtcttggccttggacggctgttcttcctcagtggcatccaggaactgctgcatataactggaggagccaagcagcatgtggcctgtggcctgcatgctgaagagggcccagcggagcatttccctgggagacagcaaggcactcaggttaaacactccccatgctatctccccaaatgtcttctccatttttctatgctggcctaaagcagttggcttcaagttagacttctttctttgtttctacttcttttatttttgagatagaatcttaacactttatctcaggttagctagaaattcatttatgtagctcagtctggctttgaactcacagaaatctcctgcctcaatctcctgatgctgttgcagttatgggccaccacacctaactttttttggtttggtttggttttgatttggctctttttgaaacagaatctggagatcctcctgtcttgtcctcccaagtgctaggtatacagacatgtcccaccatactgggctcaagctagcttttctccttaggaagatttaagtgtcactaaagtaaaatgacaaaaatgcttccaagtacagacaggaactgggaaccaggctcatagctagccatcccttacacatagttctgcctctactcttgtgacaacttttctggaaccccttgctttttcccatgcttaacttgagttctcaaaacaaagctttttattttcttgtaatgatattttgtttctttcttttctcttctatggtgctggggatggaacccagggctttatttgtgcataagcagctgacctgccatggagctatgtccccagccccatatattttttttttcaagacagggtttctctgtgtagctttggagcctatcctggcactcactctggagactaggctggcctcaaactcacagagatccgcctgcctctgcctcccgagtgctgggattaaaggcgtgtgccaccaacgctcggcatccagccccatattttttaaactactctggaccagccagattaatttacataacacatgtctaccctttgtaacactgcttcttagactttatagtcttcccaggccagttctcagaatgctggccacaaggctcccatgcctgatatcatattctaagcatagaacttggaccagacagggctgaacactgatccagagtggtttgtttatatctaaaatatttaaaatatattttaaaatattataaagatgggtgatgtaactaatttagtgcttgcctttcatgaacaaagccctgggtttgatccccagcaccgcaataacccagagtggtaatataggactgtaaacctaggatccagcactgtggaggtagaagcaggtggatcccaagttcaaagtcatccttggctacatagcaagtgtgagaccagcctgagatacatgagaccctgccaaaaaaaaaaaaaaaaagctttaactgttctggttctgagctccagcagccagaagctttgtgacttgtaaaatgggtaatgagtttgaaactaatgtgaagcacttagcatttggtctgatggacagaaaatgggacatgcagccaagagcagctggctgaatgcaacttctcccacagctattgacaagggctcggcaggggtcactaggagttgctggtcttaagaacaataaggaagagaaaacacaagaaagagaaatactgccaggccgcggtggcacatgcctttaatcccagcactcaggaagtagagacaggcaaatctctgtgagttcgaggccagcctagtcttcagagcaagttccaggacagctacggttacatagtgataccctgtctcaaaaaagcatatatatatatatatatatatatatatatatatatatattgaatttagtggcctgaggcaatggctcagtgggtaaagtgcttgccatatggccatgaggccctgaaagcccatgtaaaactggggatggctgcctgtatctgtgaccccagtactcctctcatggtgagacagagagacaagagactcctctgaagctttcaggccagcaagcctggcccagacaacagacaaacagcaaagaccctgcttgaaacacagtggaagatgagaccagcaccttaggctgtcctctgacttcgaaatgcacgctgtggtacatgggtgcccacattcacacacatatagagataaagactggctgggcagtggtgacacacgcctttaatgccagcacccttgaggcagaggcagttagatctctgagtccaaggccagcctggtctacagagtgagtttcaagacagccagggcaacacagagaaataagaaaagagaaaaaaaaaagatataaagactgaaactgaaaatataatatattagggctagaagtatagcttaatggcatggtgcttgtctagcatgtatgaaggtcctggtttaatccccagctttgaagcatgtgtgtgtgtttgtttctttttgtggtactggggaactggacacaaggccttaggcaagtgctctgccattgaactacagcctcagctttcttttcttttttaaaaatgtttatttattttatgtatatgagtactctatctgcatgtatgactttatgctagaagagggcgtgagatcccactatagatggttgtgagctaccatgtgggtgctgggaattgaactcaggacctcaggaacagcagccagtgctcttaaccgttgagccatctctccagccccctcagctttctttttacccccccacacacaatatctctctaagttgcccagtttagccttgaacttactctgtagcctaggcaagcttctaatttgccatcctcctgtctcaatttcctaggcacctgggagtacaaggccatgtatagctttatatatatgtttcgtgcagtgccttcaaagttatttctcaaattagagaactgagttttgactctagccagccagtcttaaaaaggaatgaaaataaggcctattctacaaatgaatgaaccttgaaaacagaattcttgtggggaaagaatcatgatcccatttctatgaggtctctaggatagatcaatggagcaacagaaagtagagtaaaggtgagcagggtctcgggagagggctgagaaccattatttactgagtacagcttctgctgcctggtgcaaaggttctggaaaccaaggcaatgcttgcacaacatgattagttacttcatgctagacacttcaagcggccacagttgaaaaatgttgaatgtgcagattttagcacgggtgttttttcttgaaggtctcattgtgtagctctagctagaatttggaacttgaaatgtagaccaggctagcctaaaattctcaggagatctacctgcctctgcctcctgaatgctggctgggattaagggagtgagctaccacacctgaccccttagtacatttttttttttaaatcacagtaacaccactaagtcactcagctaaccacagtttgacatcagtttttattttctctaagtttttatttttgttgttgttttattttgtttttcaagacagggtttctctgtgtaacagccctagctaccctggaacttggtctgtaaaccagactggccttgaactcacagagactcacctgcctcagcctcccgagtgctgggaccaaagatatgactcctggcctttttattttctttccatgtgtgggaggtaggggttatgcacctgagtgcagtgcccacagtggtcagaagagggcaacatatctcctagagttgcagttacatgtggttgtgagctggctgaggttggtgccgggaactggacctaggtcctcgggctgagtggttccctttttctgtttttgttttcaatacagggtctgatctggcccaggctgtccttgaactcctgattctacacctccaaagtactggaattatagttacattttcacttacaaaaatattagctgtggaaatagctcaggatatgagtatgtgcttagcatgcgtgctgccctgggtcctatccccagcacacaaaagaaagcacatgactgtcattctttgttagcccccagtgccaacccaggctttgagagagttcagctttgggtagacacaaaggacctctggttagctagtctcccaccctgctgtcccaatctactcacttcacgacgcgcttggcccggtagcagtgcaggtcataggaaagtgtgtatgtgtcatagagggtcgccttcacgttcaggcagccgatgaagtcctgggggttcagcttgtataggtcaaaggttacccgggccacatcaatcttctttgttggcttctgggaaagggatagctgtggtctcgtcttgcgctgcaagacaagtccatgttagctcgcctctgggactccttccacgccctcgacactcacccctgctggaggagactgtcacctgttctgatgggggcttccacttctgccccttctgcaggaccatgaacacagtatctcttgccagggcttggaagtattcttctgtctcaacaatcgtgccgtcttcctccagcacgagggagaagggcttgtctttaagtttcaagatatcctgggcctggaaaagaaggttggtgacatttctgccatccctatggagccacagagttgaggagcaaaggcccgcagtaaagcacttcagctgccagagagagagcaagtgagctgccatccatccgtgcgatggactgactgtcactgtgggggcccactcaagggctgtgtctcagtacttcctactcccaagaaggagtgtagtctaatagaatgggagtcatatgtgaggtcttttttattgagaaataaattacataccatgctgggcaatattaggagtcatatcgaaggccttgggcaagctaggcaaactgcagcattgaactatatccccagccttctttgtactttttgtttggagacaggatcttactaagttgtttaggttggcctggaactcactgtgtagtataggtaggcctcagccttctatttagatcttcttgactcagcctcctaagtaacttggattacaggcctacactaccaggcccagctaaactttattttgttggtggtggttttttgtttcttttgctatgtagccgtggctggccttgaactcacagagatccacctgcctctgtctcccaagtgctggggttaaaggcacgttgttatggaataatcttttggtacactgtgaagttgtgtctttgtcaaggcgctttctgactggtttaataaaagaactgactggccagtagctaggcaggaggtataggcaggaaagcaagacacagaggactctgtgaagaagggcagagtcttgggagtcatgagcaaatgcagagggaagcaagatgaaagaaggtaccactatgatgcagagggtagatagtaaaaggattaatttaagtcatatgagctagctaaacacaactctaagctatcagccaagcatttataactaataatgagtctttgtgtagttatttgagaactggctatcgggatagaaaagtctgtctatagtgtgcatcaccatacctggccactattttaaatttttatttttaattatgcgtatatgtatgtatgtgggtcccctggagcaagtgattgtgagctgcccagtgtgagagctgggaactgaacctctgtcctctgaaagtgcttttaacgactgagccaccactctagccccaacaactcatttgtaatcttgttcctgccaaagctacatggcatgaactgaagaaacaggtcattcaaaactgagggacaggaaataaaataactggcttgccatctttaaaagccagctagaagaatcagcaggtaagggtacttgcagtgcaaactttagcatttgagttcaatacctggagcccacagtagaaggagagaaatgactcccaaatgttgccctgtgacctccatgtgtaccctgtggcatgggcatgccagtgctcacacacacgcttcatagacttacagtaataattttaaatataaaataaaactgtctggatttgcaggacaatcagcagtgctccaacagagtctcaagataagcttgggggggttcttgttatgtagccctagttgggcaggaacccctatatagagaccaggctgacctcaaacatgtggtcctcttgcttcttcatgcatcaccacactcagccttagttttaatttcttttgaggcatacccagcgtatcctggaactcaattatgtagcccaggctagcctcaaacctgagatcttccatatctggtctcccagatacgagccaccatgcctggctcatttctccacgtgtaaatatggggaaggggaatgaatggccctttcagcaagaaaataaccacccacccacggttggtgtgaaagacggtagtgcactgccctccttctgtcacttgaccatgtcacttatcacacacacagctctccaggcggaaatgccaaccctccagtgccaacacctttctccgcttaagaccatgccactcatgcgcacagacttcagggctctccagaaaccgaagcagccggcaggctcaactctcagccaaggcctggagagagcctatgcagggtaccggcagatcccttaccttgcccaggaggtcctccaggctgtgagccatgatgcctttgcgaaccttccgatctgccgtgctaactcgacagggcctcgccctggggacttcccggctgggcttagacaccagctgttgggtcaccactgcagtgctcactgctacatgcctggggacaacagtttgtacagcagatgacctgcctgcggctaccaggtctgcccatgcctgcctgcttgctgagttcaggtgtgacctttccttccaaccaatcatggctgcttcaggcggctttcacaaagtccatcagaagacacagctccgatttggaggggcagggaaatcagcgggaatcttgcttaaatcagactcacttcagtgcctctgagtggagcccaaggactgctaacaagtccccaagctgtgtcagtgtggctggttcttggaccagccacattctcctgtggatggcttctgcttttgtggccttgttcaccttgcctaagaggcagggtgaaatggagctggtgtgtgtatatatgtggggtgtgtgtgtgtgtgtgtgtgtgtgtgtgtgtgtgtgtgtgtgtgtgtgtgtgtgtaaatcggcctgtagaatatgtggggactctgaggtacacagggtagaaagaaaggagcagagagaatttgggtcttggctggaattccaagtttctctctgttacggatttgctaggtgacagcatacccagcctgtgactcagtttcctcatctgttgacttggcatagttgccaccacttcccagagaggagtgaggctcctgggagatcaggaacaaaggcacctggtgcccagcctgaccctttctactccagatgtgatgggaactgtggcagtgacagagagtaagatggggactgtctagactgcaagatcttgggtgctaccctaggcctcctgaatcagatctgtgggggtttattgtaagattttgtctgttttgttttaatgggggtcttgcttatttgtttgttttttgggatagcatctccatatatagccctggtttactggaactaatttcctcatctctgtctctgtctctgtgtctgtgtctgtctctctctctctctctggacctggacctatgcagaccagactcaaactcaccaagatcctcatgccactgctccccaagagccaggattcaaaacatatgccaccatgcctggccatatcagctagaccccacatgactttttttgtcttttaaaatgggatgcctatgtagccctgactgacctgggacttgctgtgcccactacaggctggcctcaaactcagatctgcctgcgtctgcctcctgagtgctgggattgaaagcgtgtaccatcacatccagcccctacatggtttttatacctattaaagttttcaaagttctaatagatggctccccagctttccccttccttcccatacccagctctccttacctggacagtgacttagggtacaggaggctgagagacttcatggcatagtccatccttgtcagctggattgtgttggacctggatcggaggcagtaacctatctggttagagtgggaagggagggaggggtccctctctttctttctttgactcagtggatcccagcccccacacggcaaccctctgccccagtctctcctcctacccccaatttccctgaatccacaccaactacactgagtcagcgtctaccatccccacccattccacagccccagaacaactcactccatgtttctctgccccaagctctggttacactgagctgaggaacccaagtcaaggctgaagaggccctcactgatttgtccctgtccctgggggcagagtccacattatgctggcaggaggtgagtcataccacctcatgctgtgagagacttgaaagtcacctattgtccagaaactccacaggcagtcagaccttggagagcctgctctaaccatgggcccttgagcaataaagtcctcctgaggcttagaactggctaagagaactttctgtgatcttaaagactgtctaggcttgtgttcaccaataccttagctgttaccactgagcccttggcaccataactaatgtgacaaaggagctgacaccgaacttctattgaggcttaactaatttagttaggtaactactgtggtttgtgacgccaggtaccccactggacattacagttgtagtggagtaatcctgggctcatgtgccccctgcctttcctcctatattgctgttctctggttcttgcctgctgagctgcaagtctctgcacaggcagtctcttctacctgcaatgcttgtctcaaactcagagctcaacataagtcacctagaagccccagctcctggctgccaactcaggaaagccttttctggtgccctcttgcctggtaccagattgatccctgtgccctgcccttccatgttcatttctgctgcagctctcgggtccccactgtgcctgttttcagcccgtagcacagggacccagtgtgaacaccaggagtgggcaggcatggagaactgcctcctcagtgaaggaaaaccattcttcccttctgataactcgggttctccccggtcaaccttacagagttagagaccctcgtccccacttaatgctacccaagcactcttgtttcaccctgtttctaatacccaccactcacacagctccacaaaacccaccacacctgctctgtggtagccaaataccccagctcttctcctccccctcacctcctagcctcctggcaatactgcgtggccaggcacagcccaggcctcctccattttatttctatctcctgtgaagccactcccttggccccagccccagctggggaaaagagcacagggagtgagtctgaaactctccctagggatttcaagccagactgacttccagaaagccccgaaagggggaagaaggcatgtttcgaaagctctagaaggaagaggtactgcccaggacttggtaccaggcaagagggcacagaaagcaacagacggggcttgtggtctgcgttccacccctcacaccagctgtttgcgctcacccgagttgccttctttcagccctggattttctcctgtgcatgaggaataacgctcctgttgaagccaggaatggtggctcagagctgtaatctcagcacttggtagactgaggcataaaaagggccatgagtttgaagctagcctgggctatagagttaggtcctgtctcaaaaacaaaaatatagtcagacggtggtggcacacacttttgatcccagcactcggaaggcagaggcaggcggatctctgtgagttcaaggccagcctggtctccagagcgagtgccaggataggctccaaagctacacagagaaaccctgtctcgaaaaaacaaaataaataaataaataaaaaagaaatatctttttttttttgagtcaggcaagatacttgagttttattatgtctgtctaaaagcaggtgttaggcctatttgtagtgaagcgtggtgtgctggcgctgcaacacccggtgaggaagcaggaacctgatctcagagcccaagaactgcttgacagcaggccttcggcacttgccctctgaaatctcttccaccttcatcaggttaatggagtgtgcacgggcacagtgcgggcacccgtgtcttggtagcactgtttgacggctccagcagcggtcaggtcccggtattcttggcacatgttgtgtgtgtcatgttgtgagttgtagtgcagccataccgcaaagttcttcacccgcagtggggcctcaggtcgcactgcacagtgtacgatctcctcggatgacttcttcatcttgttcatctgtgcccaaaatcgggacttggccaccacatggttcgtcgcaaagatgtgcatgaggtataggagtggtgtgtggcacttccgggtgggcaagcagcgccccaccaccttgtactcccgaagcgtgcctgaggccttcgtggctgatctgtccttgttggccgccagccacaaaaataaaaggaaatatcttgcctataatgttacaatcccatccagcacttgggaagccaaggcaggaaggcctctgtggatgagaccagcctgggctacagagtgacaacctgccaagagaatggggtgggtaatcccaaatcttgggtatagtgaagtacaggactcaagagacagaggcaggaggatcatctccagttcaagacaagcccgagctataaggcctcaaaaacaaaaaaagaatattagatctatgatttattgctataaacactacaacttaaaaaaaattaaaataaggacagcaagatgactcagtgaatgagggtttgaatttgatccccagtacccacatagtacaaagagagaactgactcccataggttgtctttccgtgaaaactctccctcccttcctccctctctctttctctctctctaaatgaaaatgtttacatcaaagcctctacaaagagcattcatgttacacctagcgttggagagatgtctcagtggtcaagagcgctgacttctcttccagaggatccaggttctattacaagtacccatgtggcaattcacaatcatttataactcagttccaggggatctgactccctcttcaggcctctgtgggccttcatgtgtgtggtgcaaagacttgcacgaaggcaaaacactgtacacataaaataaaaataagagacagaaaagggaatttattcagtgtggccataccagaaaagatgagaggaccagtctcttcaaccctgtttttggagtgcagatggtagctctaggaatttacaggagaagaacaaaaaatgggatgagctggtggtgcaggcctctcagtgcttgagaggcagaggcagaggcaggggaatctctgtgggtacaaggacagcctgactgacctggtggattccgggctaaccgaaactacaaaataagacactacctctaaagttaaaacaaaaaagggaggcaataagtatgcatagctaagcattctattctattctgcccaaaccactctaattgacggctacctccagtctgaagtaagggtcctgcagtagctaggaagaggactacctcccaggctgactgttcctggctctggcaacaaactgaaaaggaaaactggttcagaaggagatcagagcagaggggccaaattagaacgaggattgtgccttccttcaggtttagaacaaccactggagaattttaggtactgctgtggtagtttggaacaagacgaggcaaaaacttattactggcaatctgtaatgtccccataaatcctttttatatctatttttaaaaagatttctatattatatgtacagcattctgtctgcatatatgcctgcaggccagaagagagctccagattttattacagatcgttgtgagccaccatatggttgctgggaattaaactcaggacctctggaagagcagccagtgctcttaacctctgagccatctctccagccctctattttattttttaaaagaattattttatgtgtatgaatatttgcatgtatgtatgtgcaccacattcatgcagtgcctatggaagccagaagaggacgacagattccctgaaaccagagttacagggggctgtgagttgctttatggtgctgagaactgagcttccttcctgtacaagaaaagcacatgctgttaaccactgagctgtccctccagccctccccacccctttgtgtgtgtgtgtacttgtatgtgtatgtatgtggacatgtgagcatgtgtggaagtcagaggatggtttgcaggagttggttctctccttctaccctgtgggtcctagggataaaactcaggctggtagcatgtccctctacctgcaaagccatctcctgggctttgtcccaataaatttcttgctctcccttggtatcttcattcttaaagggaaggacagagctacaggtacaatgggtgaaaattggcctttagttaccagtcttgaagatgagtacttaaatttttttcagaatcctttttttttttttaaatctttttatttatgtatacagtattttgtctgcatgtatgcctccaggccagaagagggcaccagatctcataatggatggttgtgagccaccatgtggttgttgggaattgaactcaggacctctggaagagcagacagtgctcttaacctctgagccatctctccagctcaagaatcctttttttttttttttttttttaaggcagggtttctttgtgtagccttagttgttctggaattaggtttgtagaccaggctggcctgaattcaaagacccacctgactctgcctcccagggtgctgggactaaaggtgtgtgccacgacacctggctaagatgagtgctttttaaatgcctatcacatctacaggcacaactgattgggaagctgattcaaaccttgagatagcaaagggatagaggtgttgatgcaacataaagacagagatgcccagattttctccttcctttagtcactgggcgggactctgcaggtccagtgaggactcactggctctcttgttaaagccttatttagagttaagccaatccttggaagaggcatcattcattcattgaacacttgcagtgtgttaccctgttaattcagcagaaagcaagacagctttaggccacacctggagcagctggaccagagaccaagtggaagcaggaatgctgatgtacacttctcatcctgatcccctggaggctgtcagcctgggccacacagtgcgagatcctgcctcaaaaacgtttttgctacccattggtgattgtacatgtactttgttctagcactcaggaggtagggccagcctggtctacaaaggacagccaatgctacacagagaaaccctgtctagaaaaacaaaacaacaacagaaacttttttgtctgatgtttcatcttgtttcttttctaataatgtactgggcagagaaaaacaaaataatgcagttgggcctggaggcttagacctgggattccagcacttaagagatagaggcaagctcattgctttgagtttaaggtccctctgccctgcatagtgagttgcaggtcagtgtggactatagagtaagaccttgactttcaaaaagcacaaggggccaggcgttggtggctcacagctttaatcccagcactcgggaggcagaggcaggaggatctctgtgagtttgaggccagcctggtctacagagggagttccaggacagcctccaaaacaatacagagaaaccctgtctcaaaaacaaaacaaaacaaaacaaaacaaaaatgtcaaaaagcacaagggccatcaaaatggctcagtgggcaattacaaacttgatgacctgagtccattccttgagacctacataatggaaagaactgactcccctaagttgtcttctgaccttcacacataccgtgtgtgtgtgtgtgtgtgtgtgtgtgtgtgtgtgtgtgtgtgtgtgtgtacatgcacgcgctctcgtgagcaagtacgctcctgatactaaattaataaaaaggattaaaaaagtaaaaccacaaacatgaaccaaaccaatgtggtgtaataacctatgtctggagaactaacaggtttctggacccagagaggtagagaagataatctaatgtcttcaggggaattagggggataaagcatcagagcataaagaaagtttccctcacttgggaagtagtggaggcaggagcatcaggaattcaaggtcatctcatctatatagtaagtaagttcaagactagcctgggttacatgagaccctgtctcaaaaaaaaaaaaaaaaaaaaaaaaaaaaaaaaaaaaaaggaagcatggtgcaacagatttgagattgaaacacaaaaatagctcaactctggggcagacagcagagtcaggggaaagtctaaggaagttctgggaaagggtcaggaggcccctccctgcttcattcacgccccaaacatctattaaacattccatgtgtacttggtgtttctggcctgaggatgactcacagaggctgacacactaagttaatcacaggaagcacagaagaaaaaaaaaaaaaaagaaaaaacaggcctaatgcaatggaatgcctgactgagggggttcttcacagtgctttggagacgaccctttgtgtctcattgaaattcatctctcaagaaacactcctcaaatgtttacacattgtcaggcactttgagctgcagggacacagaggactcagttctggtccctgcctttggaattcacaattgttgctcttccaaagaactggggttcggctctcagcacccacttaaggaggcccatgaccatttgtaacttcagctcaaccgcttccacaagtacccacacacatgtctgtcccccaaacacacctatattcttcatgctaaccttgtgacactatgagcaaaatcaaagacattaagaagccaggttcagtgccctgtgccttagtcctaacatttgagaggctaaatcaaacaggaattgaggccacttggacagcatcaagagaaattatctttgaacatatgaaatgatattatcacagaaatagaattgcaatttaaaccaggtatgtgatcccctctccccccaccaaaagaaaaaaaaatagagggctggagaaatggctcagtggtaaagagcacctgctgttcttgcaaaggatctagattgttttgttttgttgagacagagccacactatgtaactctggctgtcctggaattcactatgtagatcaggctagctggtatgtatgtctgtccacaaatgcatgcctggtgttagaggcttccagaagtgtgtgtcagaccctctggaactggagttataggtggttgtgagctgccatgtgggtgatgtaattgtacttgggccctttgcaagagcaaccagtgcccttaacccctgatacttgcttttaaggaatttgcatttttgttttgaaattacatgttggaaaagttttccacatcagtaagaaatgccatccttactatttcttcctgcagcttctcagaaatattttttaatctttttttttaagattttatttatttatttattatgtacacaacattctgcttcatgtatatctgcacaccagaagagggcaccagatctcattcaaggtggttgtgagccactatgtggttgctgggaattgaactcaggacctctagaagagtagtcagtgctcttaacctctgagctatctctccagcccctcagaaatattttaattcaaaaatctttcttagccagctctcaagccactggattacttcttgcttctgctactgacatagacttcatcttgattcactccatacagcaactagatgtgtgtatataaatgacagatagatcatagatggatggatggatggatagatagatagatagatgatagatagatagataatctcacttgctacctaggctgacctcaaactcatgcctcagtttcctaagtgatgtgattacgggcatacactattatgcctagcataaccatttgtttgccttaaaattttttaagattaattttttattatgtatacagtattgtgcctgcaggccagaagagggaatcagatctcattacagatggttgtcagccaccatgtggttgctggaacttgaactcaggacctctggaagaacagccagtgcttttagcctctgagccatctctccagcccccagcctttaaaatttttaattaattttttttttgtttgtgtatgggtgtttggcctgcatgtaggcctatgcactatatgtgtgtagtactcacctagaccagaagagggtgccagagactctggaattggagtttcagtgcatgatgagctgccaagtgggccttaatccccaaaccccagagggttgaagagggatcagtggctaagggtgcttgttcttgcagaggacccagttttgattcccagagcccacatggtagctcagaacaagaactccggcttcagaggattctgcaccctctctgggcctcatctggtaccaggcatacatctggtatgcagatatatacacgccctgagtggtgacaaacactctgtgagttcaaggctggtctacatagagaattccaggacatagtgagaccctgtctcaaccaaacaacctggatcctttgcaagaagagcaggtgcttttaaacactgggccatctctccagcaccacctcccccctttaatgatacataggtcccacatagcctagttcggcctcttaactcctgacataacacctcatgaattcctcatcctcctgcctctaccttttgagtgagtgacgagattacaaacgttcgccaccatctttgttcccagttggccctcaaggccccagggtctcagttcagatcctgccactatggatacatagcattgaaatggggaaggctgcctctgtgctgcctcagcctttgggtgacgcctcttgcttggcactgcattttcaggagcatgacatctttgctctggaccctgaccccaaggttgtgtggagctgtaagaagtgtaagggtgtctggtattggtggcgcgcgcctttaatcccagcactcgggaggcagaggcagcagaggatctctgtgagttcaaggccagactagactacagagcgagttcaggacagccagggctgttaacacagagaaacactgtcttgaaaagcaaaacaaaataaaaccaacaaaacaacaacaacaacaaagtgttaagcgcgcatgcaaccagccactgggcaattagtcaaagatgccaagtctagatagacatgcagttaagaacttagggtctagagagatggctcggtggttaagagaccagaagagtttgcatcccagcgctcgcacagtagctaacaacagtctatcttacaccctcttccagcctctcctggcacaaggaacacacgtggtgctcatagttacaaaacagacacaacacgcatacacaaagaaataactaatttttaaaatgtcttgtaatcaagtaaaagtgctaactctagggactaaattaattcctcagtcgccctactccaggagcggtaaggctggccagaaagaccaagaacgcacgcgcggaggagaaaccacagagtcggtcctcccgggatagagaggccggaagtgctcgcggagctgcacgccgggtgctggaagcctactgagccccgaggaagggctccgctcggggcttggcgtggtgggtgagccggagggtcggcgtgagcggcctgggctttggttctgaatgatggcgtctcgggcaggcccgcgagcggccggcaccgacggcagcgactttcagcaccgggagcgcgtcgccatgcactaccagatgaggtatgaggtgagccaggagcactgaggccttccccgggaggagcctgcgggtctcgggaagcgacgcgggcgagcctcacggtgccgctcccccagccagctgtcgcgtactaccgggtccccggctccggcgagcgcctcgggtctgtttacaggccgggaagcccagtggcctgccctcgcccgcctcgtgctttgaggggatctggcctgcagaggctcaggggtcgatgctcagcccctctgaatgaccttggagacatcatttttcttttttcaaatcgaggtcccgcagtgtctagagttcaggctggcctggaactcacggccttccctcctcagcctcccgagtatgcgcctggtctgaaaactaacattcttaaagaaaactgcgtgtgtgcgtgtgtgtgtgtgtgtgtgtgtgtgtgtgcgcgcgcgcgcgcgcgtgtgtgtgtttgtgtgtgtgtaagtttgcccacacgagagcaggtgtgctggcagaatgcagtgccagtaaagggtgtgtgtgtgtgtgtgtgtgtgtgtgtgtgtgtgtgtgtgtgtgtgtgtgtgtgtgtgtgtgtgtgtgtgtgtgtgtgtgtgcgcgcgcgcgtgtgtgtgtttgtgtgtaagtttgcccacattcttaaagaaaactgcgtgtgtatgtgtgtgtgtgtgtgtgtgtgtgtgtgtgtgtgtgtgtttgtgtgtgtgtaagtttgcccacacgagagcaggtgtgctggcagaatgcagtgccagtaaagggtgttggatctcttggagctatagttacagacgtgtttaaatccacctgagactagtactgaggaccaaaccccgaagtgattttgtgagatagcggcttggtatggggtccatgctagcctccaggagcggcaggtgctcttaaccactgagccatctctccagtccccaggctgtcttggaacttgctctgtagaccagactagaaatcagagatctgcctgcctctgctgccaagtgcaaggattaaaggtgtacactacagccgccctgttgaccctgtctttaggaagatgtcatgtgtactttacaaagagttttgatgtagtcatgtcttaagctttgttgtcacacaagatgaaattaattaccaccagggaatttttcatcaaattgtgctgtacttaaatatgcctaaagtaaaatacttggggtcagactcttgaagtttaaaccaacaccaggtactgagttgtgttgaacccacacaactaccttgcctcctgcagattgttaactctgctggctgtggtggttgaagagacccccacattggcttagctgttaagagcacttgctgttcttggagagggtctaaactcagttcccagcaccgacatcaggaggctcacaaccaaacacctgcaactccggttccaggggttccgatgccctctggcttcccgacagcacaggcgtgtgcgtggtgcacacacatacacatgagataaatttttaaaaagaaagaaaagccaatctgggtcctgccttcaaggagcttgagtgccagaagtgattcttttctccttacctcccacatttcctataacaaattaattttccccttaaatccactgaattattcaactggttcttggcctctttctttacagaaggatgacaagccctttggacccttctttttcaactttaggcccctgcctaatggatgctcttgttttcatgcgtacctagagcagacaccttgcttgtgacaatatgaaaaggatataattgagaccccgagaagatcaggactaagagatgtgcctccttctcatggaaactatatctggagaaatacagggagcccaaactgtgttccttgttgctaggcctccccctaatggcctttgtttctgtgatacgaagccatctttctaaccacagctcagaattcttgtcatggcttggtgagataatgtcttagtacgttgtccaggccaacctccaatcagagatcctctgcctcagcctcccaggtgccattactgttcttcagtcttgaagtctacacatgcatggtctttgtaactgttactacctctctgtgccttggcagttgatttcctgaaacacaaatactaaaaagcttattcctttgtgcctgcatgtttttgcctctgcgtagaagcccacttccactcgtctcattacctctgctgaaatagctctctcctctagatataaccttctctggctttccatctcatgctaattggttcttccctgggatagcggatatcttcggtttatatgtatttacttgagttttatacttcatgtttcccagcctctatcagaggctgtgtcattcagattgtatctatgtgtccagtaccggtgcctggtggtcggagcaatttaaatgaaaattgcttctccctctgctctgccttacagtgtgacgctcaagagtgaaatcaagaagctgatctacgtacatctggtcatatggctgctgttggttgccaagatgtgtgtgggacacctgaggctcttgtcacatgaccaagtggctatgccctatcagtgggaatatccatatttattgagcattgtgccctctctcttgggccttctctccttccctcgaaacaacattagctacctggtgctctccatgatcagcatggggctcttctccatcgctcccctcatttatggcagcatggagatgttccctgccgcccagcaactctaccgccatggcaaggcctaccgcttcctgtttggtttctccgctgtctccgtcatgtacctagtgttgatactggcagtccaagttcatgcctggcaactgtactacagtaagaaactcttagactcttggttcaccagcacacaggagaagaaacgtaaatgaagcctgcctgatggacacatgaagggagctgttcagaatctccatggactgtggcatctgtgatgttggcacctagtgcacactatcctcagattttggccttgagttctctgttaccatctgctgagatgacaaatctgtagtgtttaatttattcttgactagccacaaaccggatgaccgatgtctgtggaacacttcaagttgaggccctccagactgagccttatccttgccttctcttggtcaaattccttacttccatttatcacctcttcatcaccgatactaaaagagatctggaataaatcagtgcagaaattctacttcaatctgtaggtcatggggcaagcaacatttgggaagttgctcccctaaaggctactctgtttactgcaaaccattttaattaaaaaagaacttcaataaagttaagactgtcctgtgctttgtgtttgagatttgattgaatttcaaagttgtattgctctagaggacttagacattatgagaagaatagatgtttcctgagaacatgggactggcgctggaggagagacagtgtgaagagtcttaagtcagctgcagccctctagctcttaggtgagaacaaccctgggatgggggttgctggaggaatggctcaggggttaagagcactgacagaggacacaggtttagttctcagcacccaaatggtgacttaaaaccatttgtaactactgtcccagggcatttgacgccttcttgcctccggatgcactaggcagacatgaggtacaaaaacaaacatgcaagcaaaacactcaaaaaattaaaaaaaaaaaacatggtctctcaatgtagctgtggatgtactagaactcactaagtagagcaatctgggctcaaactcacagagatcctcctgcctttgcctaccaagtacagggatttaaggtatgtgccaccacacttagcaatatatatttggccagacatttaatctccacccataaagcagcttatttggcttggattatgagctcccctttttatgcttgtttgagacagggcttctctatagctctggctgtcctggaacttgcttcgtggaccagactggcatcaaactcaaatccacctgcctctgcctcccacgtgctggcattaaaggtgtgtgcatcactgcccagctgggttacgagctttctatttggttatttatgtttattagacttttatgtgtctaaaactgagtaataatacccaaaaagaatctgcttgttggggctataattttccagtaactgacaaataaacctgagtcaaaataagaagaaaggggtttgagttctttttcccttttagtttctgtcattgattattccttggcctgcttgtttggggaatgtgcaaaggaagctgctaactctatggaagccagaaaaagaaaaaggtgagattccagtgtcgccactaggggcagactgccagtgacgtaacttcttcactcagcccctcccaaagcttcctccacctccaaaaatgtcaaggactggtaactagtacatgggcctttgagaaaagatagcagggagcttccagtcttgggggaatgtgcagtgaacataacatctaattgtgtgagagacccacagctgagatacagtttaagagtcacacctagaagtcttcacagtaaataaggcagcaaaggtgtgcatccctttgcactggactttattatgcctagatgatgctcagatctgtaactgcagagatctggccactacagttgtaattcttttgtcctggaaatgtgttgcttgtacagtctgcaaagctaattcctccagctatcccactgatcatttctttggagcaggggcagtttccagacagaatttctctgtgtagtcctcgctatcctggaactcagagatctgcctgctgtgtgctgggactaaaggcatgtaccaccatgcccagctcctactgatcatttcaaagataacttcttgtgcctcaggcctattgctgttcctgcttcctgtgtttacgggaacacagctctggatcatccaccctgcaagtttaagtccgtccccactccatatacacactgcctattcggtgaacctcctatagataccctgataagttccctagcttctcagtttaacacttagaactctccctacttaaggggaggcctctcagtggtgaagtgctcctctagcatgcaccgaggcctgggtgtgactccctcgtaacaacaaataaaaacaaaccttttctgctcttacattcctttattaatctttaagatgccacttgtatgaaaattcctcaattttcaattcctcaatttgaagaacaactttaatttgtctggttgctttcaccttattcctttattattattttctaacaagacagagcccagaatgaccttaagctatgtggctgaggatgactttgaactcctgatgctttgtctctacttcctaagtgctaggattacaagtatgcaccatcactcctggcttatattactcgtcccccacccccaccccatagaccaagccttgacctttatttttaaaagctgcatatttattctgctttctgacttagtgtttgtgccttcaacactttcacaacccttttctcttcctcaataaggaaagcctgcttgatcctgtcacggacacacttggcacacaaggagccaccacagcccctgctgacgtgcttctttgtctagacagtctcataaggactttgggtctcacagcatgaaccccttcaagtctgcctgggcacacaccacatggggattcaggtgctttcccagtcttcttggtataaaggtaaacaatcctgttgccaggggttcgagacagcctagtttcgttagaggctgtattgtaggaaagcctacaatggtatgtcaaacactggaccattcgagtgcctctaagcaccatccctggaagaggaagagctgtattatttgttttatattgacagaattgctactttgtatgtcttctgtagctcattagtgttcactctgagcttattgaatgaattaacttgcccttttgaggacaaaggtctgtatttcacagaaaacagaactggactgaagcagaaagaagcccatggacaagggagtttgtcatgagcttagctgaggcacacattgctaacttcaaacaactgtgatgcaaacaattaaaactgcagtgcagacccccctgctttctcccaataaattttccacataagttctgtcccccccccaaaaaaaaaacctgttagcacattattagtcaaagagctaaaacctgtttttggtcatcagaatagtgttttactgaaagtctttgaataccttgtagactaatttcactcattgtgaaattagtcaacagttttaaaacactatcatgccaacatcagtttttgttagttttgtgcttggaattctgcttttctagccttttcctataacttttttctgcctcatggttatggtgttcacaagtcctcttgatttagtgggaagaatgctatttgggttagaaagttcagctgggtatgggggtacatgtctaatcccaacctttggcaggttgaggcagcaggaatactctgagtttgaggccagcctgacatacatacatacatacatacatacatacatacatacatacatacaaacactatctcaaaaaacaaagttggagttaaagtatcaaccacaggactaagatcaacctgcctgttcacaggttgctttaaagctccaatagccggttggagagatggctcagaggttaagagcactggctgctcttccagaggtcctgagttcaattcccagcaaccacatagtggctcacagccatccattatgaggtcaggtgccctcttctggtgtgcagatgtacatggaagcagaatgttgttacataataaatacataaaatcttaaaaaaaaaaaactccaatagccaaagtattatgaaggcattctaaaatttttaatttattttcattttatttttggagacacagtttctctgtgtaaaaaccccaactgtcctggaactcactctagaccagactggcctccaactcacagagataaacctacctctgcctcccaagtactgggattaggccgggcagtggtatcgcccacctttaatcccaacactcgagaggcagaggttggtggatctctgtgagtttgagaccagcctggtctgcaagagctagttctaggacagcctccaaagccacagagaaaccctgtcttgaaaaaaacaaaaaacaaacaaaaagcaagttaatctggaattaaaagtgtatgccaccacacctgatgtaaatttaaaattttttttaaattaatttttctatacacaggtgttttgccagcatgtatgtttgtatagcacatgtgaacctggtgcctaaaaagccaaaagagtgcatctgatcccctgggactggagttacaaatggagtgctgccatgtgggtgctgagaattaaaaccaaatcttctagaagagcagccagtgagtgcccttaactgctgcagcatctttctggctcttgtgaaggcattttatggagtctgtaacaccatatgggtatcaaaagccgtgtggtcacctgatctttgcagcaagacaggagagagaaggtgtttgagagacttgagggatggtctagacaccaaagaaaggtttgtgtgttgtgagcaatgactgattattaggttgagattctagccagggagtttactatctcttgtttgatttattacattttattgccatcctaggaattaaatagggttttacatttgctaagcaagtgttctctcaacatccttatatcccctccccgatttctgacacttactatagctcagcttgtccttgaactcagcaatcctgcctccatagtgccgggattatggatgtttccactcccagctgagcctgttcttcatgtgttgcatgggcattgcagatctctcccattgctgtgaggctgaaataaatgtggatgaactttgtagtacatagtcgtttacatatcaagatgcctttccttggtaaacagtgttatgcttgcctctcaaattgggagtgtcttcctgttttaagaataatcctcttccctcttttcttcctctctcctcctttcccctccttctgacagggtctctttatagaatattcctgcctctctctgccttccaaatgttgtgtaggtgtacatcaccatgcctgcctagttagaatgacgtctcagaatgctggaggttaatttcacattcttagtccacattatcctgactggtatgttcaaggtgccctgacagtaggtacagacccagcagacctcaggaagtgaccattacagcaagatcctgtagctgctactcatagcctgttgggagcccgtgcataggaaagaatggcaagaaaaaatggctgccagagggcgtccccatagtatactctgtcactaagcatgcatgcttcagagcttgccaaaacctcctgagtccctggcttggtccccaaataaaggtcattagagcatgaagtcttggtcaactgattgagactcctttggagagtgcaaggcttctatgtagatgcccctgttgcctcctattctgtctaatttcttactctccctgttgacagctgccaatctcctttctcctaggactgtgcccactcatcactcactgtgataagacccctttccttttctttgttccttcctggtctgcctgcctgctcccccccccacctctcccttttcatggtctcatgaaaactgctctcagactcactatgtagctaaggatgactggagctcccaatcctcctgcccctacttccctaattctgggattacaagtttatgccaccacattccactttctgatttccttccttacatcttaattccactagctttatcacactgctaaagctaaaactttctttgcaaaatgagatccagaaacccacagactctccatagtaggttctgacagggaaacagctgacattgtatggtgttctgccttcccatttctttgctgtgtgtgtttttggacaagtctcggtcttcctgtatttccttccttccttccttccttccttccttctttttccattttttatttgaattataaacacgattgttttacatgttaatcccagttccctctccctcccctcctcccctaccaccatccccaactaaaaccctacctatcacatatcctttctgctccccagggagggtgaggccttccataggggtcctcagggtccgtcatatcctttgggatagggcctaggcccacctccgtgtatcttggctcagggagtatccctctatgtggaatgggctcccaaagtccacacctatgctaaggataagtactgctctactacaagaggctccatggatttctgaggtctcctcactaacacccacattcaggggtctggatcagttccatgctggtttcccagctatcagtctggggaccaagagctccctgttgttcaggtcagctgtttctgtgggtttcaccagcctggtctggacccctttgctcttcattcatccttctctgcaactgtattccagttcagtttagtgtttagctgtgggtgtctgcttctacttcttccagctgctggatgaaggctataggatggcatataagtcagtcatcaatgtcattatcaggggagggcatttaaagcagcctctcctctgttgcttagattgttagttggtgtcatctttgtagctctccaggcatttccctagtgcctgatttctctgtaaacctaaaattttccctctattatggtatctcttatcttgttttcttctattcttcccccaactcaacctttctgctccctcatatactcatcttcccttctcattctcctagctccttcctccccttcccaatttgctcaggagatctggtccctttccccttctccaggggaccatgtatgtctctcttagagtcctccttgttacctagcttctctggctttttttttttttttaagatttatttattatgtgtacagtgttctgcctgcaggtcagaagagggcaccagatcccattacagatggttgtgagccaccatgtggttgctgggaattgaactccggacctttggaagagcagtcagtgctcttaaccgctgagccatctctccagccctttctaaaactggacattagcctagcctcaagggttgtgatgctaacaatacaagctaaagagagcaatgggcatgatccaaagccagatagttcagcaaatattgatttcatcccttcccttttcggaacagactccagccacaccaaatatgtaaaccagcaagacaaaaaacagcaggctatcttttcccactttttgctttgtctatgtttgttggacaatgtcaaactatgtagcccagaccccttctgtaactttccatgtagaccaggctggcctccaactcaaattcccagcaatctacctgtctctgcctctggagttctgggaataaaggtgtgcatcaccatgcctggcctcaataagtccttgtcttatggtcttcttttctcctcctttacttcttatcctcctccctctctcttccctttccctttatctttatttctttattgtcaccgtttcttttctgcttctcttaccatgctttaacaaccatcaggaagcaactaaccaaatgttctcatcttgagagtcaggtcagaagatctaggagtcaagaaagacacgtgtaaaatagagctcacataccaagcattagaaaactcgcagtggcactaaaagatggctcaggggataagaacacttacatggtgactcacaaccatttgtaattccagttgcagacaattcaatatctccttctgatctctaagggcacccagaatgtacatacatacattcaggtaaaacgttcatatacattaaaaataataagccttaagaattagaaaagagggctggagagatggctcagaggttaagagcaccgactgctcttccagaggtcctgagttcaattcccagcaaccacatggtggctcacaaccatctgtaatgagatctggtgccctcttctggtgtgcagatatacatggaagctgaatgttgtatacatattaaataaataaaatcttaaaaaaagagaattagaaaagaaaaaaaagcttcatgtgtgtgtggaaacctccatgaggtagaatgggataaaagctcttgattctatgcttagtctggctagatcacctctcctgttctctggatctctctgacttgactgtagaggttggctctgtacaaggcagcactgtcagcagatcctgttggttcaaagaggtaggtgaagccaagagactacaggtgacagcagattgggagggcccagcacaggtgcagctcactggaagggtccagcacaggtgcagctcactggaagggcccagcacaagcgcagcaccttgggctatagactttgaccttgaccaaatcactttcctctctgcttccttatttctctccttacgaaattaaacaaataatcctcatttacctgcctcacaagttgtttagagggatcaaatattgctgttgctttgtgaactgtaagtagataagctatttagatgagatatatcatgattgacattcactcagccatgaagcacaggcttcctgacaggcactgctaggtgatggagaagaacaaatgagtctctccatctctcttttctcagcactaggattgaacccagggccttgcatacactaagcaagtgcactgccgccgagcttatctgtttttacctccttttaaaaatattgtttatgagctgggtggtggtggcacacttctttaatcccagcacttgagaggcagaggcagggggagctctgtgacttcgaggccagcctggtctgtagagtgagttccaagacagccagggctacaaaataaaaccctgtctcaaaaaacaaaaacaaaaacaaaaacaaaatcaacaaaatgtatgaatgcaaaaacaaaaggatactttagggtcgcaggtagattgggaacttcaaatgtgggccaaaagaaggctgtggccctgcttctgaaggatggtccagaccgaatctcagaaggacaaagtgtcggggctaagatgtagcttcgttgatagagttcatgcatgcctagcacgaaatcctgggttcagtctccagccctgaagaaactggatatggtggttcacgcccgtaacctcagcacttgggaggtagaggtagaaggaccaggagtttaaggaccgggcccagcctacgatacgtgagaacctgtcttgatgatgatgataatgggactggagagatggctcctgtgtaaagtgctgcataaacacgaggacctgagttctggtccccagcaccagcatgataccaggccatggcagaatatacctctagctccagtatcgagtagaggagacaggtggaccctggggcttgcctccccaccagcttagctgagacagcaaggtctaggttcaatgagagatcctgtctcaaaacaaaatagagataaatcaggaagacaccctggtgtcagctctggcctccacacgagcctgcaagcacacctgcccatacaacacacacacacacacacacacacacacacacacacacacacacacacacacacacacacacaccctggtgtcagctctggcctccacacgagcctgcaagcacacctgcccatacaacacacacacacacacacacacacacaaacacacacacacacacacacacacacacacacacctaaatttttttaattaatttaaaaagaagagacaatagcaataccaagatgcaatgacccagtggacatgcccctaactgtcctcccatcgtggagattgccaaggtgaggctgaagcagctaaagtctccatgaatgagaggaagctgtgagatgatggggagggtccccacaaagcctggagccttgtcagccttttttcctgcaccggaaaccacaggaaaccaggtcagtgtgtgtggctgctccctctgctccagagctcagctgtcagttttggggcttcctgagcaagctgtattatttagtttgtccttgaagctagagaccactttgacctggagtcgagaagagttggaaagccagctagctggggagccagagtgtacatggataacacaagaagtttatttgtgttatatgttggcctatttttttgcctaactttaaatgatgtaccaattgcttctttatcttcagcattcagtatagacaagtgccgaatagctgttaattctcaaaatggaagatgtgtaactgtctatgacagatccaagttctagaacatgctaataggcgtgccagatctgcagtgtttgaggagggttaagaaatgggtgcatttccacaacagggtgctggtccctttcctgttacctctccgccacaccaagagccccctgtgacgtgagagcagtaaggctttctaccaaacaggtggacaagtgaattagtgttcaccagtgccctggctcaggagaaggaatgtgacccagcctgctggttaggatagaggaggagcttcagggcagggtggatccagggcagggcttggtgagagcgcctgctggtactgtgtgctcctcacagctccagggccaggccctgctgccctgctgccctcctgcaaacaacaaagccatggtctctgtgccctgatcctgcagaagcttatggggagccccagactgacaacctggttcctgcctctcctcttactgctcttcagcctgtctatgtctgctgaggttggctgtccctacctgccacgctggaccagccactgtctactggcttcccatgtggtaaggtcctgctgctaggtgggggacacttgcttggcacagagtagaaactccccagtctcctgtctgccctttcctacctctgaaatccctgtgatctagtggcagcctctctgtgtccaccatgtggtcctgaaggttctgttgggctgaggtccttggaaggatgagaagaaaacagctggggtggatggacagtgggctgtgggtctcagaaccggatgatcggcacaaagagttcagggagcatgcacctcctgcagttcccttctaaccacacctctctcatttcttttccgcactgggctctgtacctccaggataagaattctgccggtgagtcctgtttcttgttctgcttcactccccatgctcccctgagttccaggtcccagtgagccatccatggccattttggttcccagcctggggagtagtctagtatcaatgggaaagtgcgggactagtgaagatttgcattgtaattctgcctcagacgctatgactgcttttcactctcccatctgaccctcggtctctgagcacaggagaagaggagaaatgattatgcgatgaggacagtgttgctctctaccaatggggaggataaaatgagcaccatggctaatctccaggcactctggacaagctggctcttcttatcaccggtacctcacttgaccaggaacaataatcatctttcatactttattgtgcattctgctttaccaagctttcccccatgccttatgtcacttggtccttatgacaaacccactttgagcagtctggcacctgcccaataacttaaccaagattgtacctgtaagctatatcagagcccagcccgaaatccagtttggagaaggtaggtgggaccccttaggccctaccatttttcttgttcctctttttgtttttgttttgagatagggcctcactatgtaataacaaagcctccataattatttatttacttattttggttcttcaagatagtgtttctctgtgtagtcctggctgacctggaactcactttgtagactaggctggccatgaactcacagagatccacctgcccctgcctcctgagtgctaggatcaaaggcatacgccatcaccacccagctgcctccatcttttaatccttccatctcagcctcccaaggaatagcttacagccatgtgttattgtgccgactttctatcatacacaggaagttctgttcaaactccctgccctaccctcctagccacgaagccttggcactggctctgctgtttgtgcctgcgtctgatgtcacggccttcaggtaagaaaagcagcctttgagccattcttagtgaaggcatgaatcagggacagttgaaaataactagctaggctgacacaagtgtcaattgtcatattgggacatatatttgtcttgttaacctcaagagggacttagagcatcctagaataacacatcatgacagacattcatgggaccactatatctttgtggaggtgccatctttcatctggcactaattcctaaaagccatgtgttctgtagacttgctattgttaccttccctctgtaaaatgggactgccactctgctgcttcattgcctcacttggtacatacggaacacacactttaaaaccagcagtcataggcggatggaaagatagcttagccatcaagagtactagatgctcttgcagaggacctaagttcaattcccagcacccatatcaggcagcatacaactgtaggtaactccaactctaaggaatctgatgtttctgatctcttaagcctcatttatatgtgccaatacatagatatacatacacatgcaagtaattaaacataaaataaaatcacaaaaagttagtttaaaattgttcaccattagctgagtgctagtggcgcacgcctttaatcccagcactcgggaggcagcggcaggaggatctctgtgattttgaggcccgcctgttctatagaatgagttctaggacaggctccaaaacaatacagagaaaccctgtctcaaataaataaataaataaataaataaataaataaataaacaaacaaactaagaaagaaatcatgctacaattaacactgcttttgtgggaggtgtgtgtgtttgtgtgtgtgtgcgtgtctgtcttttttctttgttttattttattttttaaatatcttatgttagaggtggagagatggctcagttgttgagacagttgctgcccttccagaggatctgtattagattcccagtaccctcatagtggctgtatacacaattgtctgtaactgtagttcagggaatccagtgccttcttttgacctcagcaggcaccaggcatgcgcatggtgtacattcatatgtgcaggtaaaaccttcatacacataaaataaaataaataaacctaaatatttaaaaaagaaatgaaatatcttttatttattgtttgacagtctcatacatgcagacaatctatcttggtcaaaaccacccacaattcctccctcccatgcacttggataactcccaatacatcactgctccacattcatgtctcttttaagatcttgtgtgtatgagtgctttgcctgcatgtatatatgtgtattacattcatgtagtactcatggaagcatcaaatccctgtagcactggagttatagatggttgggagccgccatgtgggtgctgggaaccaatcctgaatctaagaacatcaagtaggctgaactgctgatccatcttcacacacacacacacacacacacacacacacacacacacgcacgcacgcacgcacgcacgcacgcacgcacgcacacagagagccatcaaattttgagacagaggcttactgtgtagtcctggctcacctggaacttgcagagtagaccaggcaggtgtcaaaatcaagagatcagcctgcctctgcttctcaatacaagtataaaaccacgccttgctctgtttgttttgtaaattcacggagtccagctatgagtctgcctgctggaatgttgactgatctggttggcttgaccttgtgcagttcctgtgcaggtaaccacagctgcagtgggttcatgaacacgacagtcatgccatgtctagaagacttcaaaacactcttccacatcttctggctctcacattctttctgccctgtcttctgcatttgctgaaccttgaaggggcttaattctgttttttactgaatcataatttttgccaccaaagaagctcaatatcattgctacccttgatttctataaacattctacccaacattgggccacatctccagataggacaggggccaagaatgcccaggtaagaagtgtgtttccctcccacaggccttcagtggaactggtttcctctcttggtgaagaaatctaaaagtcctcctaagtctgaattctactggaggcgtagaaggccagcatcgtcccaggtaccctatcatttacaacactatggccctcagcccctttctttcttgccccttattttgttcttcagcaacaagagggagtttgaaaatgtggagattacgcactaagggaaactgaggccaaaggaatttgaacaacttgcctgttaaattaatttgtctctcagggttcagaggcagagttagaaagcacacttcctggatcctggcagacttcctggatcctggctcacatccactttgttctctgcttgcagaggaagctgctaagcagcctttccctgtctcaggaagaccatagcatttccaaccccttcccagccatctcccatggagggccacactgcaaaaggacccagcctttggacaccggaggagtagaacattttcctagagcaggttcacagaagcgtggaggtaggtgcgaggagcaacaagctggtggtctcagccttgcctgagccagtctctgagtcgtccttctccctaggacctgaattctcctttgatttgctgcctgaggcacaggctattcgggtgactattcctccaggcccagaggctagtgtgcgtctttgttaccagtgggcactggaatgtgaagacatgagcagtccttttgatacccaggtatggagttccatgccttgcaaagagacagattacacaatttctcagcaaatgggatggggtgggggggttggggcccagacaaggaaagtaattgtccaaaacttccttacaaaatgagataagtagcattaaaaacttgttgggggcgggggaggactagatagaaaagatggtgggctatgttacttgagaagagagaccagaaagaaatgagaatggtatgtattgccagggagaggactcttggtgtgattcgggtcaaattaagagactttgctaagggctggggacatacccgttggtagaatgtttacctagcatgcttgatctctggcactgcacagatggagtgggaggtggaagcaggagaatcagaagttcaaagtcttccttagatatataatgagtctgaggtctgtttggagtacgtgagtcttaagagaaaaaaaaaaaaaaagctttgttatttcctcaggaggttgttccctgaaaatttgacaaaactgtcatatgtgtcttggtacttttccctttcataccttttgtctagccctcagaaatgatattccccagtctcacaatccaggtcttgacagcctactttaactgccactcctcacagatgccattgtatgtagctgggcatcacatgcagagactggagtccttcagcttgcttcatgtctacaatggctataccttctaacaataccagcctttagtagaattgatgcttctaatactgcatggcatacagctggaactcaagaaatgtatcgcctcttccctaaccctactgcttgtagagtacctcaagaaaatggccaggatatagaacctatgagagggagaaataagttccatggagtaggaaagtaaggtatattgtccaaggtcccggcttcatcctgccttgtccatagtgtccatgcttttccttaggtgtctctttttctttccctttgtggttttatcaacagaaaattgtgtccgggggccacactgtagacctgccttatgaattcctcctgccctgcatgtgtatagaggtgagcacagggaaatgtggattagccatggctccttgtccaaccgcatctccagtaagtcaacttagaaaggactcgggaaggatggagatgacttggtgatagagcaggccgtacacactaaggagagactccaaaaaaaccgggctactgggatgcttaggaagctggtgctggctaggctatgagccacaccagaggtggaagggtcctaggcaattccacctcagccctgcctggatgctgtcttctctggtccagatttggcttcagctcacagacacagagtagacagccatcatcgctgtgttcttgagagccaaggttgcatatccctctcaaagtcatgttctttccaaccctccatttctccttgtaagtctgaagataaagatgttttggacatttttagggggaagacaggtactaaaagaagggtatggacagagacctgggaaacaagaagctgacagaggagacaaccagtggactggactcgtagggagtccagagtgaggagtcaacacagggtactgggtacttgagaaagtcaacaatggagctcagagagcctaaaattgagtgatgcaacctcgggaattctctgctgctgggaacagttctttggagacaggaggggtcatcccctctcatttgccactccctcctatgctctggaaccccaacacttacacacactccacagtgctgcccattattgtctgacagctcaagctgtttgatgacagtaagccagagccagaatgaagatgttaggaaagccatttctaacaaacactctctatgctccccaggcctcctacctgcaagaggacaccgtgagatgcaaaaagtgtcccttccagagctggcctgaagcctgtgagtgttgtttgtattaatcccgatgcacattcatactccccctgctgtgtgtgtgtgtgtgtgtgtgtgtgtgtgtgtgtgtgtgtgtgtgtgtgtgatggaggaagaccccagatggcaggaggacattttcttcaggagccaggggaagatgccacagctgtcagccaacagatgagctcctgagtgctgaacattatgaatggaaaaggatcgcccagcgagtgtgtgagagggccaactgtagacctaggctcacttctcagttctgccataggttatccccatagcccctcccctttccctcatctgtaaaatcccaaactgtttttgaggcttgaatgaaatattttaggggaaagtggctggtgttgtctggagtgctgtatgtagaatggtgaccttgaggagagaggacacttagacacaagtaacaaagtctaacgtacataggcagggttatcatcaagggtatcagcccaggacaggagaagcccagagacactgttgtgagcagagagggtgtcacaggatggcacttgactgagctcaggaactggacaggaattagaaagaagaagcagggggaagacagactctgtgaggagcctacacagattctggatacaggcgacagtaagggctgttcagtgtggaggcctggatggaaaggtaaagccaaggaagctgggtagaaagggagggagcgtggagtgcttgcctgttgaggacctgtgaagcctggaattgtgctggaacattggtattttctcctggtgtcctggggtggggtggggagatggagttacagctgggctatggcctcactcttgcacctgccacctgcccagatggctcggacttctggaagtcagtacacttcactgactacagccagaacaaccagatggtcatggctgtgacact

SEQ ID NO: 3 - DNA, 9227 bp, Cricetulus griseus
  misc_feature (1)..(9227): expressed and VP-generating type-C group 1 ERV ERV-C 109F
  misc_feature (1334)..(1336): Myr motif
  misc_feature (1851)..(1868): PPYP motif
tgtgaaagacccctctcccttagccctttctttctcaagtttgtctcctcttcctcctgtcggcggcttccccgatccccacccccggtggcctttccccgcccggcccgagaacaagcaccgggtggggccggcccgagaacaagcaccgggtggggccggcccgagaatgagcaccaggtgggccagcccgagaacgagcaccgggtgggctggcacgagggcgagcaccaggtgggtcagcacgaggacaaacaccgagtgagccggcctggagctctgcccctgagcccccgccccgcccgaagagaaacactccgtcccaaggtctccgcccccaaggtcagccatcaggaaaagggggggaattgagtctgctgtaccagacaccagaccttgagaatatgctgatctggaatggctctgtgtctcatttgaaccatccaatggaaatgattctgtatttcgcctcatttgaaagactctgtgtttcacctcatttgaataactctgtactttgcctcatttgaataaccctgtatagcgcctcatatacattgaccaatgggaatagctctgtataatgcctcattagaattatccaatagaatccttgctcctagcttgcgccttttttcctatataaggaccccttttcccttggctcggggcgcttagccacacagaagctaagtcgccccaggtacctgcgtctccaataaagcctcttgtttttacatccagttcgtggcctcgctgattcctgggtgtgtgggtctccctctacgaaagtgcctcttcggggtctttcatttgggggctcgtccgggattgagacccgcccagggaccaccgacccacgtctgggaggtaagtgttgtgcggatccgctgttttgtcttgtctggtctgagtctgtcttgtgaattgcgcttgcgtttgtagtatacagctgtgtacatttgtaggcggatccgaggagggactgacgggtccgaactcccgaccgcggctccaggagacgtcctggtagcgtttgaagccctcaggaagagggatttgtattttgaacttgggaagccctcagggtgagagatttgtactttgaacttagatctatgactggacattttcccagtctctttggagaaggccctcggcttgagggatttgcaatctttactggggacgaggaaggagggcccccttcctcgactctctctcaattccttctgtcgactctctgtcgaaaccgcgctgcgaaagtctgttctgtgttattcggtctttgtcttgtagctgtcatttgtgccctcctaagcctagaaactatggggcaaactgtcaccactcctttgtccctaacactctcccactggaaagatgtacaggaatatgctcataaccaatctgttaatgtgcgtaaacgcaaatggattactctttgttcttcagaatggccgacctttgatgtaggctggccgcgagatggtacctttaacccccagactatattccagataaaagagaagattatggatcctggaccacacgggcatcccgatcaagtggcttatatcgtcacttgggaggctttggttcaggacccccctccctgggtacgtcctttcttacatcccaagggcccctctctccttcccccctctaaccgctccaaccgacccattccttcggcccctacacctcccactcctttgattcctcccaaccccccttcccattccaacctttaccctaccgtgatgaaagacactaaggctaaagaaaagaagacacctaaggtactccctccgggagaagaccagttggttgatctattaacggaggagcccccgccatatccgccactgccgcccccaccagaggcagaagcggactccgccgctgccttggcggaagcggcccctgacccttcaccaatggcttatcgactaagaggtcgtagggagcagcccgttccagattcaaccactctgcccctccgaactgggctgaacggccaacctcagtattggccattctcagcatcggacctctataactggaaaaataataatccttctttttctgcagaccccgtgaggctgacatctctcatagagtcggtactcacgactcaccaacccacctgggatgattgtcagcagcttttgcaggtccttttaacctcggaagagaaacagcgcgtgctactagaagcacgaaaaaatgtcccaggagtaaacgggcagcccacccagctacccaatgaaattgatgcggcttgccctcttgaaagacctgaatgggattttaccaccgaagcaggtaggacccacctgcgtctctatcgccagttgctggtagcgggactccggggggcaggacgcagacccactaatttggcccaggtgaagcagataatacagggtgcggaggaatcacctgccgcttttctagagagattgaaagaagcgtacaggatgtatactccctataatccggaagatccaggtcaggccaccaacgtttctatgtcctttatttggcaatcagccccggacataagaaacaagcttcaaaggctagaaaatctacagggatatacactccaagatttgttgaaggaagcagaacgtatttttaataagagggaaacacagacagaaagagaagaacgttggaggaaggaaactcaggagagagaggaaagactaagacaggaagctgaggaaaaagaggttgcgagagaccgtaagcggaataaagaaatgagcaggttattggccacagtagtgacaggccagagacagaatagacagagggatgacagaagggggccccacctggacagggaccaatgtgcttactgtaaagaaaaaggacattgggcaagagaatgccctaagaacccccgggccaagcttccaccgccaagggtttctgacctcctgaacctagaagattagaggagtcggggccaggagcccccccctgagcccaggataacactgcaagtcggggggcatccggtcaccttcctagtcgatacaggggcacaacattccgttctgaatcggtcacccggacccctgagtcacaggactgcatgggtacagggagctacaggcggaaagcagtaccattggactacaaatcggcagctccagctcgcgaccgataaggttatgcattctttcctccatgtgccagactgcccctaccccttactaggacgggacctattgaccaaattaaaagctcaaatacactttgagaggtcagaagtcaaagtcacagggccagagggaattccccttaccatcttgacaatgtccatagaagatgaatatagactccatgaaaagaggactaattcgaacaatcaggaaacccttgatcactggcttgcggaatttccccaagcctgggctgaaacaggaggaatgggccttgccattaaccaggccccaattatagtaaccttaaaagctgccatccttcctgcatccgtcagacagtatccaatgcctaaagaagcccgcgaaggaattcggccacatattaaaaggttacttgaacaagggattctggtgccctgtaaatctccttggaatacacccttgctacccgttaggaagccgggaactaatgactaccggccagtacaggacctgagagaagtcaataaaaggatagaggacatacaccctactgtccccaacccttacaatttgctgagtggattgccacctaactatacctggtacacagtcttagatcttaaagacgctttcttctgcctccgcctgcatcccaccagccagcctatatttgcctttgaatggcaggacgcggcccttggaatctctgggcagctgacttggactaggctacctcaagggtttaagaacagccctaccctttttgatgaagctttacatcaggacctggcagaattccgggttaggtaccccgctctaatcctcttacaatatgtagatgacattctcctggcagccaaaaccaaagggaaatgcaaggaaggcactcaagccctcctccagactcttgggagcctagggtaccgggcatccgccaagaaggcccagatatgtcagaaacaggtgacctatttaggatacaagataaaggatggacgtcgatggctaacggaagcccgtatgcgagccatcttagacattcccaccccacaaaatccccgccaactgagagaattcttgggaacggcaggcttctgccgcctatggatccctgggtttgccgaaatggcggctcccctctaccccctcactcggccaggggttgcttttaaatgggaagagccccaaaagaaagccttcaccgacatcaaaaaggctctccttgaatcaccagccctgggtctaccggacttagctaagccatttgaactttttatagatgagaaggagggctatgctaagggagtcctcacccaaaatctggggccttggagaaggcccactgcatacctctccaagaaattggatcctgtggcatcgggatggccaccctgccttcgaatgattgctgctatagccctgctggtaaaagattctcacaagctaaccttggggcagcctttgaccatacatgcccctcatgcagtagaggcagtcatcagacagcctccagatagatggcttactaatgcccgaatgactcattaccagactatgctgttagacaaagaccgggtccacttcgggcctttggtgactctgaacccagccaccctgctccccctccctggggagcccgaggctcatgattgcttacaggtattggccgaggcccatggagcgagatccgacctgactgaccagcctctacctagcccggaccacatctggttcacggatggaagcagctttttgcatcaaggagaacgaaaggcgggcgcggcagtcaccacagagaatcaggtcgtctgggcccaggcactcccccctggaacttccgcacagagggcagaactcatagcactcacgcaggctctaaaattggcagaaggtaagaggctcaccgtgtatacagacagtcgttatgcctttgccactgcccatatacatggagaaatttacagacggagggggctgcttacctccgaagggaaagacattaaaaataaggaggaaatcctcgctctcttaagggctcttcatctgcccgctgccttaagtatcatacattgccctggacaccaaaaaggggattctttcgaagcaaggggcaatcgaagggcagacttggctgcccgagaggcggccctgaccacagacaccactaacctcctggctctagagcccaccaacgaccatcccttcccctcatgggactatgaacaaagagacatccaaaccctagagaaattgggagccgcaaaggaaccaaacggggattggacttatgaaggaaagactgtcatcccctaccgggtaaccaagtacctagtgacatttttacataagatgacacatctgagctccaagaagatgcgggagctcctcgaacgagaagaggaattcaatttccttttggggaagaacgatattctaaaacaggtaactgagcaatgtgatgcgtgcgcccgagtcaacgcatccagactgaagcttcctcccgggaaccgggtcagaggctaccggcccggaacacattgggagatagatttcactgagattaaaccaggaaaatatggatacaagtatctattaatttttgtagacaccttttcaggatgggttgaagccttccctactaaacatgaaacagccaagatcgttactaagaaattgcttgaagaaatctttccccgttatgggatgcctcaggtattgggaacagacaatgggcccgccttcgtctcccaggtaagtcagtcagtggccaccttattggggattgattggaaattacattgtgcttatagaccccaaagttcaggacaggtagaaaggatgaatagaacaatcaaggagactttaacaaaattgtcgcttgcaactggcactagagactgggtcctcctactccccctagcactctaccgcgctcgtaatacccctggaccacatgggctcacaccctttgagatcctgtatggagtacctactcctatcattaactttcttgatcaagatgtctcagattttgctaactccccttctctccaagctcatttacaggccctccaactagtacaacgggaggtctggaaaccccttgctcaagcttataaagaccagagggaccatcccaccatcccccattcctaccagatcggggacactgtttgggtccggcgtcaccaggccaagaaccttgaaccccgctggaagggaccctacatcgttttgcttaccactcccaccgcactcaaggtagacggcattgcagcttggatacatgcttcacatgtaaagccagcccaacccaccgattcagccactgcatcagaatggaccgcacaccgcactcaaaatcctttaaagataagactctctcgtacaccctcctgttgattggttgtctgtttaccccccatgtagcaactaacccccacagggtttataatatcacctggaaaatagccaatctagggaccggggaaatagccaacctcagcacttatatagggactctacatgatgggttccctcctctctatgtcgacctatgtgacttagtagggtctgattgggatccctctgaccaggaaccattcccagggtacggatgccaccaccctgggggaaggataggaacaagaagcaaggatttttatgtttgccccggccataaaccaactcatggctgcggggggccgcaggaagggtactgtgcaagatggggatgtgaaaccacaggggaggcttactggaaaccctcttcctcttgggatttcatcactctcaaacggagggagatcccagggtacgcagggaaaggaccatggagatgtgggcaaagagcctgcggaccttgttatgatagtgccggagggggaggttttcaaggcgccacccccggaggaaaatgcaaccctctcatcctaaggttcacagatgctggaaaaagaactacttgggatagtcctaaggtctggggactcaggctgcaccgagcagggaaagatccggtgactttattctccctgtacagacaaattactcccctaagccaacaatcagttgggccaaacatagtaatagcggaccagagatccccaacccattttcaagtccctaaaccccctaccgttcctaaagctatcactcctacaccaggtgctgtcaccttctcccccaccccagatgccctaaacatcgagataaccagagaccctccaggtaccagagatagattattacaattaatccaaggagtttaccaagccttaaatttttcagaccccaacaagactcaggaatgctggttatgcctagtttcccggcccccatattatgaaggcgtggcaatactgggcaactactccaaccagacctcagcacctaccagttgcggagctgctatgcagcacaagctcacaatatctgaggtctcaggaaaggggctatgcataggcaggattccttcctcacatcaagaattatgtaaccaagtagagccattatctcaggacagccgataccttgttgccccttatggaacttattgggcttgcagtactgggttgactccctgtgtctctaccactgttctcaacaccaccattgacttttgtatattgatagaactttggcccaaagtcacataccaccaacctgaatatgtttacagcgtactagagaaatcaacccgatataagagggagccaatatcctttaccgtggccctattattaggaggaataacaatggggggcatagcagccggcatagggaccggaaccgttgccctacagggaattaatcattttaagcttctacaacaagccatgcacacggatatccaggtcctagaagagtcagtcagtgcactcgagaaatccttaacatcactctctgaggtggtcctgcaaaacagacggggattagatttattatttttacaggaaggggggctatgtgctgccctcaaggaagaatgctgcttttatgcagatcatacaggaatagttagggatagcatggccaaacttagggagaggctaaaacagaggcaacagctatttgagtctcaacaaggatggttcgagggatggttcgctaartccccctggttgactacccttatatccacgctcatgggacctctggttattctatttttgatcctcatatttggtccctgcattctgaacaaactgactcaattcatcagagaacgactatctgttgtacaggctttagtcttaactcaacaatatcatcagctaaagcaaatagatccagagtatctagagacctctgaatgaaagattccattcagttacaagagaaatgggggaatgaaagacccctctcccttagccctttctttctcaagtttgtctcctcttcctcctgtcggcggcttccccgatccccacccccggtggcctttccccgcccggcccgagaacaagcaccgggtggggccggcccgagaacaagcaccgggtggggccggcccgagaatgagcaccaggtgggccagcccgagaacgagcaccgggtgggctggcacgagggcgagcaccaggtgggtcagcacgaggacaaacaccgagtgagccggcctggagctctgcccctgagcccccgccccgcccgaagagaaacactccgtcccaaggtctccgcccccaaggtcagccatcaggaaaagggggggaattgagtctgctgtaccagacaccagaccttgagaatatgctgatctggaatggctctgtgtctcatttgaaccatccaatggaaatgattctgtatttcgcctcatttgaaagactctgtgtttcacctcatttgaataactctgtactttgcctcatttgaataaccctgtatagcgcctcatatacattgaccaatgggaatagctctgtataatgcctcattagaattatccaatagaatccttgctcctagcttgcgccttttttcctatataaggaccccttttcccttggctcggggcgcttagccacacagaagctaagtcgccccaggtacctgcgt

SEQ ID NO: 4 - DNA, 30019 bp, Cricetulus griseus
  misc_feature (1)..(30020): 5′ of SEQ ID NO 2
tggttctatcgagacagctacatccgcttctccacacagcccttctccctgaagaacctggacaagtgagctttccctccacctcccttgactgcccaggctagtgaggaggtcagcagacaaacagaacagtgggctctgcgggcagggaaggcaggcttttcgccgccactcatcctctgccctgaaggagcttgccaagagggtgcccttgggttaccgaaatgagcaaaacaagaactcctgctaatcaagggcttacatcctagcaatgagcttgagtaatcatggctaacatgctagaaggtgatatgtgttaggaagaaaatgtagatctgggtaatggcagattaagagaaacttgagaggggaaaggcaacttgcagtttgctcttgggatgagaggaaaccttgaggagacctgggttcaaattctatctctgcagcatccagattgtgttcctctagagacatgaaaagagacttaagcctcagtttcagttagagcccagtggtcccatctgtaatcccagcacttgggagtctgagtctgaaggattttgaggtcattctgggctatataatgagacccccactcaagacaaaacaagccaggtgtggtagctcctaactgtaatcctagaacttggctaaggcaggagccaaaatcaagccaggtacggtattggaggcccgtgatcccagtgcttagaaggtagaggcaggacaatcagttcactgtcaccctcagctacatgccaagttttatctaacctgggcaacatgagaccatttcatagaaaaagaccagcctccgtttccacatctgaagactggagcagcaatacactgtagctgcttatcatcaagtacttcacatgtgataagttgttcctgcttggttataccacagccacttctcaatggctcacaatgaagtgagttattgccattttaaaaagagatgagaggggcaaaggttaaatgacttgcctgagatctcatagttgatcatcggtgaagctgggatttgaccatccagggtcatcgtccccctgccacacataggaatggtgaactgaaactaggcccaaagcacttgatacttactgtggcctgcaggcaacactggagcattctgagaaatgactagccctggccgtgggtgaatagggagtggggcagggagtgggggtggggtggaggggtgcagcttctgtgcttctgtcctgttgatcttccagcaggggcccctctcagttgagatcccctagacggctgaccacagggttctgcctgcagccctgaggttgggctccagggctgagcctgtgtctctgatccactctagctctgtgcacctatgtaacaattccatccagagatacctggagacctcctgtcaccggcaccggatgctgccctcggacaacatgtggtccagccagaggtttcaggcccacctgcaggaaatgggtgccccaaatgcctggtctagtgtcattgtacccggcatgaaggctgctgtgatccatgccctgcagacctcccaagacactgtgcagtgccgaaaggccagctttgagctctatggggccgactttgtgtttggggaagacttccggccctggttgattgagatcaatgccagccccaccatggcaccttccacagctgtaactgcccgcctgtgtgctggtgtgcaagcagataccctccgtgtggtcattgaccggcgactggaccgtacctgtgacacgggagcctttgagctcatctataagcaggtgagatgtcccagcacctcccacaggcaaccctacagcaaagccctggctggggtctgctgtgagacagagttcaagactgactctacacacggggcatcttaacacagacacgtcccactggcctgtctcctcatctgtggaagatactgtcctttgagagccattaatgccatgagtttgtagtcataggtagtattttcagaggccctgggacctgctcagtttccatggtaattccaagcctctaggtagtaaccttattctctcatctgtaaagtaagactgtacctggctcctcctttgtgtgctgtgagaatgggtggtttgatcttcacacaagggtttgctaaagtaccaagctggaggacatagaggatatgaggccatgaacctcaaggcctgcctatcaagagttaattattagtgtctatcattgttataacgattattatgttactcatcttcttccaaaaacagatttgatcttgcttatttataataagtatagaattgcttggggtttttgtttttgtttttgtgggttttgttgagatagggtttttctgtatagctttggatcctgttctggaacttgctctgtagaccaggctggccttgaactcacctttgactgcctctgcctcccgagtgctgggactaaaggtgtgcaccaccattgcccgacctagaattgttttttattcagccaaaaaacattaccttgccctcgtggtctaattctctctagaaacacccttaggagcacaccaggggcatgccctatagaaatcctaggttttctcagtccagtctagttgacaactgatattagccaccacagctggacatggggctccaccttccacctcccagtatttggaaggctgagacaggggatcacttttagttcaagggaagcttgggttacacagtgagttccaggctagcctgggcttcacagtgagaccctacttaaaaacatcatacaaacaaacaaacaaacaaacctttaaacgtatgtttttaagggtttctgaattctgaggacagtttttaagatctttgagctaagattcaccaaggtctaggtttgctacaggaagggaaaacactgatcacctgacctgggtggcctcatgtttcctacaggtcctgatcaccttagaataatgtgctggcaaagggttccgtctttgttagggatgccatcactaggtgtccagggtggaatccaggcctgcgtttttagcatgtgcagttctactgagctacacctccagcctaaaaaatgtaaaggaggagatgcatgcaggtgtgcatacacctgtaatccctgggcaacagaagcaagaggactgtcacaggttcaccaccacctggttacagggtttataataaggtcctgtcacaaacaatgtagtcttggaagagaggagaggaaatggggaagaggaagtaatttgcagtttaaaatagagcaaaattgccgggcgttggtggtgcacacctttaatcccagcactcgggaggcagaggcaggcgcatctctgtgaattcgagactagcatggtctacaagagctagtttcaggacagcctccagagctacagagacaccctgtctcaaaaaaaaaaaaaaaaaaaaaaaaaaagcgaaatttgtatggacatggcaacacgtgcctgtaatcccaacacttagggggctgaggttggaggatcaggagttcaaagttatccttggctgtatgtgagcttgaagccaacctgggcctacagcactgtctgttccacaatctgtctttatctgtttgtctccttatgttgttcagctcggtctgttctctgaatgtttgtctcagaacaaacaaaattgaatagggctgggcatacgcacttttcaaatgtcctttgtcccccaggtatctttcattgctgatttggttttgagcttgagggtcagtgtacaacaaggtggttacaatggtggctttgtctgtctctgatctcctttatctaggacagtgccactgctgtatccctggcaccctggtcctttgcaggccaggcaggccagcctgcaccaccgccccacactgttttatctgtttttctccttatgttgttcagatcggtctgttctctgaatgccctgtaaactagaagataggctaacaagctgatggggttcagggttgagatttttggcaaaaaacactcatgtgatgctaggtacctcatgtgactgtcacaaggcacaaccaggaatctctagttcccaatgccgaatttgaccttagactaaggtggctgccaccagagccacatcctcccctttgtagcttttttcatttttctttacattatttattgtgtttgagtttgtacatgtgtgtggtcacacatgccacagcacacatgtgggagtcagagggcaacttatgggagttggttctctcctcccatcccatgggtcctggggcttgaactcagcaagtaccttataagctatctcaatactgtttgcctccaaaatgtattactttgtgtagctgtcccttttctgttgctaataacagaatactacagacagtgtaagttacaaagaaaataggctcaattgtatccatcttttgctgcccatggcatgaaaacacttgcatcccaacacaggccagaggtcgctttagtgtgaagcaggattgagtgcatgtctgcatggctaagactctctacttctctggtttcctgatccctagggctctgggactctagatcctctggaccccttgataatagagggagcttcctgtttctcagaagtgccttttgaccatatatcccagaaactgattccatccatctctgccgtgtgtcactagtcactagatggcgtcactgtcatctctcactagtgtctgagatggccttgtcttctctcctgcccacagcctgctgtggaggtgccccagtacgtggggatccggctcatggtggagggctctaccatcaagaagcccatagcagcttgtcatcggcggacagcggtccgctcatcactccctcatctgctggcccagcaaggctgtggggaaggcaaggactcaggaccccctatccacaggtcagcttctaggaaagatgctggggccaggagcctgggacacactgagaagccagactctgcggccaccacctcagtccccggaaaggggaagaaaggcaaggcaaaaagtgccacagccctggtctgcatcaccctgcagaaatgggagtcccacaacaccagggtgggccccaccttcaacaggttaatgtgtctgaaacagcctgaggcctggggtagtaccatgtcccccaaaccccgcagtgttcccaaggccatttctgcctgctctccaagccctccccaagcatctgggcttgccctcctgccaaaaggccaccagtgatagcaagtatgaaccaaatatctttaaatacataaccaaatgagtattacaaagtagtcaccctgccaggcagttagaccaaaggctcggtcctagagtgcgcgcccagagtccagacccatgctgctgctctagccagcctttgccctcacctttctctggagaaaggtgctgccaccatgcccttccccattcctaaccagccccctcagccctcataacgccctagtgaggtaggtgctattgtccccattttccagccgaggtagcagcaagtttaaggacgttgcccgaggttgcacagctcagaaggggcagagctgggatgcagacccaggtctgttggtctcccaaccctgtgttcttcccactgcctctggaggaggagctgggaggggctccatctgcccttaccttgtatcccccacctttacacatgtactgtggaacaattggtcaggctggggcctcacccagatcctcacagcttcccttctcccacagccccgtcctacctccctagtctccattccaaggcctggctgccttcttcccatgtgctccgaccccagggccgggtcctcagactaccgaatggccaactggtgggctctaaggctctgtcaaccacaggcaaggccttgatgactctacctactgccaaggttctgatgtccttcccacctcaccctgatctcaagctggcacccagcatgctgaagccaggaaaggtgggcctcgagctgtgcctcacaccctggcgggtagtgctgagcagtgggatcggggctgaagggcacgaacagagggcagcgctcggaccatacagcgccccagggaagggcttgtcttctccagaaccctgttccaagacagaggcctgatcatatctctttccctcccctccttgcaccgaggctgctattcccctgcaccttcgaggcccccactttggaagtgcctcgaggcctctgccctttgaagttggacctcttcctagcacccacaggaaagtcacggccaaaggcaagttcaaggccatactctgcgacaaagccagggctgaggcataccccaagaagaggctgagcctccccaaacccttgacccttattctgacatgccggacactgagaaccatgggggataggaggctagagaaacccctgctctgatctctactgccccatcctggatccagcatcaaattaaaaaaagcaattaaagttctctggacttggcttgaataatgtgcggctaggctcataaaagagttgaccagcagggcctccatcagcaagggccacagtccccacccagcgacagacattggctttctctgcagggagacggatgggtggggaaagagccttcactatacagatgatgacactgagacatggcttgcctgagaccacagcaggcggaaagtcagccatcaggagtgccccttcccaaagacaagctgggctggcagaagagcttctgatggtcacagaacattatgacaagagacgggctttccctaaacctcagcctttctccggagagtatgtccctcagtgaggggtcggtcaagacacctcaaactgcaaaacccaagaaacaggtcaagtgtggcatttcctacctgtagagcctcagctgctggtcctccaaaagcggtatctagctgtagcgtgtgaatgcttcctgagtggggcagggtggagaggagagtgcggtgttgaaatccaatgacctgtgtctcccaaagtcagaagagctcatgcctgcacagtggtgtgtgtctgtcctcccaggacttgggaggcagaggcaggtgcatctctgaattccagtccagccagggatataccaagaggcccagcctcaaaagcaaacaaacctcctcgtgaccaggagtacagcagatgccacctccagacctggccactgttcacttgagcagagagcacagtccctggtcaacacttgctttctcagcagatcctcaaaagcagacttgtgaggtaggacttttattattttaagtccagagagcggtcacctgcccaacgccacacagtaagtgagtggtttaactgggatttatcttaggttggtaggactaaggagtcaaagaccttgaccgtactcagcaccaagcccactgtccttgagctgggtcacggcccttctttctattttccaattgggggttaagcactgtctatcggtgagagagccgcaggcactgcagggtccgagagaagtacgagggtaggggtgccacaagttcactaggggggtccctggatctggtgcctgggaggaggaggcggtgcaagtgcaggtgcaagggcatctgggccacctgggaaggacgcaggcgaaggcgtctgaggagagcttcgtccagcacctggagtgggaaagacagcagcccacctgagttccagccagaggagcccctggctctgatggacctctctggtctgcaactgccatcattttctcaacaggcaggcagggatttctctccacacagagctaagttacgtttcagctccttttgtgtttagtgaagccatgtgattaagccactcaccaatgggatgtgaggaaaacacagacctagccctcaaagccccgattcacaaaaacatccagtctccccttatggccaggaagcaatagcccttttctccaagtggctacccagagtcaggtccactcctactgtatgcccatttgccagacttaatccaagaagaaacaatggacttcagggcctgtcagaggtcagctccccactccttgagctaacagacagaaggaagcctgaggaagtcacagcaccacagaggcaagggtggccccagaggccaagcctgttcccccaattccctaaatacagagcaggcatgtgggcctgaagacctacctgtctctggagcttggtggtcttggctggccacagaaagcgctggcccagaacggtgcccactcgaggagcataggtgtggtccccaagcactgggcagagctgtagagccatgtgcacctgaagctgactggggaacactgaaagatggggggaggcagttgctcttcttgatcatcaaggactgtccccattcccagagcctgtgcatctatgaggtgacctctgcctccacagagggccatgagcagatcaggacaatgacagctggcaaccttcccagcctgggggcattgaacccaacagaactgaagctcctgggaataagtatgtgggttgggtgctaaactgtgggtgtgcacctaaaactctgcctcctgctgagcccactctcagagtccccagatgctcttttttttttgttttgttttttgttttttcaggcggggtttctctgtggctttggaggctgtcctggaactagctcttgtagaccaggctggtctcgaactcagagagaggtgcctgcctctgcctccccagtgctgggattaaaggcatgccccaccaccgcccggccaccagatgctctttggatgttgcaaaacaacatcttcctcataggcttcatttcccctgcagagtactgttcagccctacctgtcagtggctgcagctggaccagagcacagccacagcctgtggccatcacatggaaatggctgagggtcctcttgacaccttctaggatgtcctttcgagatggggatgtcacggggatggcctaggataccagtcaagaatgacttagcacacacgtaggagcccaattgagccacccctgctgccggctgacttaggacagggcctcagaacaggcagcagcaacttgcctgttagaggacaggggaatagccaccccatgcacatagtgcagactcctctgtaaggcaaaacctgaaaggactcctagtggcttctgactcacaagatcaatgccatccatgcgttccagcttcagggccacgtggatagtcccctcagaaggctcagggatgccatcagtgatgccactgagaaagagaaaggaagtctcagagcagccagctgggaccttccttggccctgggagtcaccccgcatctctcaccagtaggtggctgtgggcctctgtgttctccttgagtgaacgaagaacttctgcaagcggcttgctgtctgggggcagctggagagaagcacaagcccagacgcctccctgaaagaagaggacaagtcacataaagcctcttggttggaaaaaatgagggccagattggctgctcctgcagaggactagcactcggcacccacatagtgggtcccaactgtaattccacttccaagagagctgatgccctcttctggcctctggggcaccgggcatgcactgtggtacacagacacacatgcaggctggcctccgggggcacaaggcatgtgtggtgcacggacatacatgcaggctaaacacacatttaaaaacaaatctttggtcttttttaaaggagaccctcccaccaaggggctggagaaatgaatcagtggttaagagcactagctgctcttccagagtacctgggttcagcacccacatggcagctcacaactgtagctccagttccaggggatctgagaccctcacacagacacacatgcaggtaaaacaccaatgcactatatatatatatatatatagatagatagatagatagatagatagagagagagagagagagaccaccttcccacctccccaaatgaacaccaggactacagtccagacctaagaaacaaatcctggccaggcagtggtggtgcacgcctttaatcccagcacttgggaggcagaggcaggcgcatctctgtgagttcgagaccaacttggtctacaagagctagttccagggcagcctccaaagctacagagaaaccctgtctcaaaaaaaaaaaaaaaaaaaaaaaaaaaaaaaaaaaaaaaaaaaaaaggggggggaaataaacaaatcccaagaccttttttctggcccccagagaactcagacaaatgccctaagagttactacacatgccaggcacctgaataggactcttatatacagtgggtgtaaaataaaggcccaccacactcctcacttactttccaggtgctcgcacaacctggagctcccggtgtttgagccccagggcctggctcagctgtggcagcacagaaagcaaggtcagctccccaggtctccctgagaagtaagagggagaaaacaagttgccagagcaccatccaacgaactcctccccaccagcctttccctccttgtccctgcccatacctgtcacaggcagaccctgtggcttgttcagtgtcaccagaggtcctgaaatattcggtagatgtcagcaaaagcagggcagcctgtgtgcatgcagcctctggttataagctcccaagcccactccaaactgaacttcctgctccccgcacccagggcctttgcacctgctgccctgagctttcattaatattgggtcccttatcacacacctccctataatgtgtgcccctcctacagtccttgactgtttggtctgagaccagctcaaacaggccggcctcaaactcaactataggtaagtctggtcttgaactcttgatcctccttgcctctgtctcccaagttctgggattacaggtgtggtcaccactcccagaaacagcagagattcactattcctcacatcagaacgcttgctctctcaaggcaggagccagccatatttatcccagtactgtgtagcctcctcattgctacaatgcactgaagttcagcaaagttgtcattcctaaaggcacacagcatgctgcttacagaccccaggaaactaacttctgctcccgagggtcttagaatgcgaggcggtgtgaaggtttcctatgcctggcggagggaatgtgacggctacaggggtccgactccagggggtcggaggctccctcaccttgctgatccaccacagctgccctcagcacctcagccagctcctccgggccgaggttctctgttcgcagaagcccggggaagggctggtcctccactgcgtccttgcacttgctggaaccttgaggttgaggccgacccctgagaaaaacgacagaagccggtacctccaacgcccacgctgcccagtgcagccttttcgtacacaaccctcagaacccccggctcggctgacagttgtggtccacaggcccccaagactacagaaggggcatcagacgctcggagcgccaaccatctcgtgtcgggtcacacaacgcggctgggattagaacccatgtcctgatctgagtgcccgacctgccctccccttgtcctagcctccctcctacacggaagtgttgagttataatcaccgggcctcggtgccgaagcctgcatctctcggccgtgcgccagccgccagccggggccgccaggtgccagacacacaacgccaaacgcggaagacgcccatcgccgcagcaagcacagacgcatgcgtgtccacggactgccccaccgcgtgcatcctccgtgcgccgattggtcagcctggctgtcaatcagagcatcgagcaggcggagcttcgaggacggaagagccaaactttcctttggctgaggaaggtaaagtacggtctccggctctgctttgggggaaactgaggcaggaggcgcggttatttactcgcggtagaggacgtgtcttagaatagaaagaggcgagtttgcagatagattgtttcagttctcaggacccttaatctgaaacccatccttgtaaagcagagggagaaggtgaagccccacagccttcgggccagggcacctgcatggaaatggtctgcgacattaaccatcccatgactgtggctcaggtggtagagagatttttctaggatgcacgagttcctgggttcgatccccagcaccacacgaagaccaagatcacagatacagactgagttggagaccagcctgggatatgtaaggcccttctatcacaagaaaaacaaactaggccgggtgtggtggtgctcttcttcaacagaggccaacctggtctagacagtgagtttcaggacagccggagctacacagagaaaccctgtcccccccccccaaaaaaaaaagaaaagaaaaacaagcttggtgcggtccacatagtaagttccacactggccagtatgttgttaattccaaagatggaatgagtgtaaggaaaaaggccagccaaagaaacacactttggctctgcaaccagaaccaaagaaatgcacccttgtcagaagccagccttggtgacgcccccccccccaatgcctagctagccgccagctagcctagcatcccacccctcaggttccaggacctggcctgagaagcaactaacaccttcccccatttccttagttagtgcttgagataaacataggagattccagatacctggcctgagaagcaactaacacctattctttctccacacaggagattccggatacctgcctgagagcaattaacattcattcctcccccacctcctttttctctgcttcctcctttctctataaaaaccccttgtaataatcaataaatgggccttgacaagaaaacctgcttggtcccgctctttctttcccgcccatcattttcaggcgtgatccacctcggactcaggaattaccggagccccgagggccggggtacaccctgaccctgaaaccagtgccagagaaacacactttggccctggaatcagagccagtgaaagaaatacaccctggctctaaaatcacagtcaaaaggctaaaaaggaccagtgaaaggaacacactttggccctgaaaccagagccaaatctaaccctaaacctgtgcagaaaaccaaccaatccctgagcaggagatctagccattctgagctcagggattgaaatctgaccaatccccaccctggaaatctccctgggaaaactctgcccccccctaagaatccctatataaaccctgtgcctgttcagcttcaggctgcctggctgccctctgccactggattcttgaatgaggtttgggtaagaactttcgccggagcagagaccaccctacacactggctctccaggggactggagttgaactgttacaatttaaccagggaaaccctctcctccccaagcagagctgatacactgaggaaggcctttccctgaagcagggctgtaagccgctatgcttactgtgacctgtggcattccttggctcctaaacgccagaatacctttccatcccagctgtaacactcagtattctgtgactccggagtgccagaatactttgcatctgagccataacacagtattctttggctcccgagtaccagaatccattccttcccatccaatccgttgaaatgcttacaatgagaaaaaccacgaacccaaatcattgagaccaaattaattaaagtttttttttcattcatgtacgagactgcctcccccaaggcctgatttgagaggtcagcattggatgtgaggaagacaagggggttttgttgttttggttttttgagacagggtttctctgtgtagctttggagcctgtcctggaacttgccctgtagattaggttggcctagaactctcagagatctgcctgcctctgccacttgagtgctaggattaaaggcatgtgccaccaccgcctggcctcacctggatcacacagacacatatatacataattaataaaatacagacctggcctttaatcccagcactaatctacatagtgagttgcaggacaaccagagttacatgaccctgtctcaaaaaaaaaaaaaaaaaaaaaaaaaaacaacctaaataaataaaatgcagaaagtaacaatatgtacataataaaaataatgaaataataacataattgaggaacagtggggcatggtggcatgtgcctttaatcccatcacttactgatctacatcccaagcctgaaggaccttgctgaaagggctccagcactagactgttgcttctggcagagaaagtggctctcaacctgctggtcaacgacccctttgggggtagaagggccctttcacaggggtagcctaagaccatcagaaaatacagatattcacattacaattcataacagtagcaaaattacagttataaggtagcaacaaaaataattctatggttaggagtgggtctccataacatgaggaactgtattaaaggttgcagcattaggaaggttgagaaccaccaatttaggggcagatgctgcctccttcacagcactcagccacagttgtgttgtgtccttgttgaggttctatccggtcctccaaagttcagctgtgggaaactaaatctttaacttcataataatggtaattggaggctggagtaattagtaccagataaaatcatgaagagtgtgcttccatgatggccattaacagctttctaaggagagcagagaaaaaaaccttttcctcgttgtggaatgacttcctccgtgttggtgtgcaccagaaaggccccctgcttgtgctaagtggagctatgccatgctcttggactctgcagcctctagaaccatgatccagatcaactgtgtgtctgtgtgatttgttttatttttattatgtgtgcatgcctgtgtgtctgcgtaagtgtataccatatgtgttgcaggtgcacggagaggccataaaaggggcatcagatcctccgaagctagagttttaggcagtcgagagcctccacgtgggtgctgggaactgaactcagagcctctgcaagctcagccagtgctcagccagtgctcctcagtttgtgtgcttgttttcagtactgcagatggatccaggaccccacacgtgccaggcaagcattctctcactgagctgcaccctcagactggtctcaggtggtttgttatagcaacagaaaacagacatgtggaaggagtgatggcgtatcttgactgagaggaggaataacttggctactcgcacagcacatgtctgggcaggttcacaaggaggcttccagggacaattggcacctaagaaccctgacctaatgaatggatgaaccattcagaatttggatgggttcttgggggggggtgagggaactgtggtgggtggggactgcttggaggaagtagggtgtttgttgtgggtggctctcggaggctatgtcttacattggctcctttctgttttggttctttcaatttcctgttgtgtgctattctgctacaaccgcctcacccagtagattgcagcctctgttaatcaagacagggtcttactgtgtagccctggctggcctggaactcatagagatctgtttcttctgcctcttgagtgctggaattaaagacgtgtgccaccaccaccacctgacctagaggacaactttattttttttaattttttatttattatgtacacattgttcctgcctgcgcaccagaagagggcaccagatctcattatacatggctgtgagccaccatgtgggtgctggtacttgaactcaggacctctggaagagcagccggtgctcttaacctctgagccatctctccagccccctggatgattaattgtagaagtttcagttctttctttttgtttttgttttttgagacagagtcacactacgtatcttcaactgatctggaacatgctacgtagacaaggctggcttcaaatgtgtgtcaatgttcctgcctctgcttccagagtgctgggattataggcatgtccctaaatctgttatttttttaaacacttatttttgagggggagtcggtggtggtgctgttgtacgtacatatgtagagatcaaagggcatcttgtgggagtctgttctctccttccaccaatgggttctagggactgaattcaggttctggagcttgacagcaagcaccttaatctgctacaacatcttgttggtactaaatccatggagaattgaaaggattgtttattgttgggcccgggaatatagctctgtggtagtagaatgcttgcctaatatgtgtgagattcttaaaaaatcattgattgttttcttaaaccaactttttaatttgttaaaaccaacttttaaaggatatatctaggcatggtggcacacagcattaaccctagcacttgagagatagaggcaggcagatctcctatgaatttgaggccatcctattctacgagttccagtacagccaggactacaggactgtgtgatatgcacaacacaggaaggactgatgacaaatttggtaaaaaaaagaaagaaaaacagcaagtggtttcacattgtccggcttcacctggagtgggtgactgtgggtactcctgaagcctcccaggagtgggtgactattctgtcagactgtgggtattcccgaagcctcctaggaggcctcctgtgcatcacagaagtggcttgagcaagaacttgggacagactgggcaagtgcacagctataatcccagcactgcagaggtgagggctgatgggtcaggagctcacagttatccttgactacaggaagtctgaggccagtctgagctatgtgagactctaaaacatacatgagtcagtgagttggctcagccagtaagggtgcttgataccaagtctgaccacctgagtctgatccccagaatccacatggtaggagcagagaaccaactcccaggagctgtcctttgacctctggacttatgctattgcatatgcataccaatacataactccctaaaatacaaaatacatatgtttgggctttttgtttgttttgttttgttttttcgagacaggtttctctgtgtagctttggagcctatcctggcactcgctctggacaccaggctggcctccaactcacagagatctgcctgcctctgcctcccgagtgctgggattaaaggcgtgtgccaccaacgcccgactgatgtttgggcttttttgatggtttttatgtgtgtgtgtgtgtgtgtgtgtgtgtgtgtgtgtaattagaaggagaaagagagagagggagagagagacagagagacagagacagagacagagacagagacagagacagagagaggagagagagccaaagtctggtttgtggcccattcaactaagcagacttctcctccaccaatggtgtccaccaatggcccaggaaaggccatttccccacttgaaccagcgcctggccctctggagggttagttcctttactctttggaggtctccctccctgccccatagcaggctgccctctcttgctcccttcccacaggcagctaccttcatcccagtgggctatgagctcctggaagaaaaggaggagccaaattttgcctaggctgagggctcagtgccaggtggcagccacagtcaactgctgaacttgtgaaactggaccccaggagaagaagcctgggacaaacatcagcttgcatcagctccctctggctcagttacagctttgcagcaggtgtggacaggctggtgcttcagcaatgggaaacaggactgatgcaaaccaggcctccaggaggccaagccctggagccagcctgcagtgaaccagcctccagccacatctacaactgtgtgagggcctagaatagaaagtgtacttgacctgtctgtccttgagtgctcttctcttctcacacttggctccaggcatggggcagcttccagcgatggctttccagacaagcttgagtaacttggccctttgtcttagtgttttctaaatccaaataggctttacagggaggattgtgaaattatgttagctttggatctgagagccagactgaaacctggcttcctacaggcctagtctttcctgcttaggtcagttacctgtcactaataaatgggggctccttttcctaccagccaccaagatggctgaagtggaggagaagaaatgaaccttccacaaattcacatactacaatgtggacctggatcagctgttgacaggttctaggaactgctgatgcagttgtacagcaccccagaggtggtgtctgaaccacggcctgtggcagaagcagtactcgctactcaaacacctgagaaagctcaagaaggaggcaccaaccatggagtaacccgaggtgctgaagacccacctgagggacacgatcatcctgcctgagatggtgtgtgtgtacaatggcaaaaccttcagccaggtggaaatcaaaccagagctgatcaactgctacctaggcgagctcttcatcacctccaagcccatgaagcatggccagcctggtattggtgccacccactcctccagctgcatccccctcaagtagctgtggccaacaaagactcatgtttaaaaagaaaattggaagccaggcattggtggcacacgcctttaatcccagcactcgggagacagaggcaggtggatctctgtgagttggaggccagcctggtctccagagcgagtgccaggataggctccaaagctacacagagaaaccctgtctcgaaaaaccaaaaataaataaataaataaataaataaaataaagaaaagaaaagaaaagaaaaatgggggctcactctgagaagacctgactcctcctcctcctcagaactctctggtttgtttttgcagtgctggggtcactgtgccaggctagagactcactgcaatctgagggtggaagtgctgagcaggaacagggaactgtgtataagctggcataggcattgataaattcccctgtagatggaccaggacctttcaacccgaacacatggaaagttattgtaaaatacaacagttgggaatcaaaagagtggtttgatccaccttacaggcaaatttcattcggaaactgacaacacatatggatcatgtggtctgcctgaatatcaaatatggcaggcctcaaaaatcaactgcagattttaatataacatttaattattcatctattaattaatttattctgtctcataatatcaaaccttatctattaaaagggagcagggctggggatgtggctcagttggtagagaatttgcctagcatgctggaaaccctgggttccgggttcaatccccagagccacataaattggatgtggtgtttcacatctgtaatgctagcacttaggaagtggaaacaaatggatcataagttcaaggtcatcctccactacataataaatttggagccagcctaggcttctgtatctagaagaaaaaacagggcaccactgatgcaactcattgcataaaagcaattgctgtgtgagcctgacaatccaagctcaatccttagaaccaagagtggaatgaaagttgtcttctgggccatgcacgcctttgatcccagcactcgggaggcagaggcaagtggatctctgtgagttcgaggccagcctggtctacagagtgagttccaggataggctccaaagctacacagagaaaccctgtctcaaaaaaaaaaaaaaaaaaaaccaacaaaaaaaagaaggttgtcctctgacctccacacctgcaccatagtactggcataccaacacacacacacacacacacacacacacacacaccagtaatgacaaatgctatctcctgtaattctcggcaaatagtttcaaacaaagaacacaggcaaagatgaggtattggcataaataatttacttcaggctctaataccatacattagtggggaatgagaacaaaaggaggaattgtctgacttcccctggggatcacaaactctatccattcggcccagcaagggcgccaatgaaaggtgaagaagccacgattccatccaagtccagcttggtgaaccggtgagtttactaggttacttacaggtggggcctgggtgactcaaaatcacaggtgactccctccaaatctgcatcagtgacatcctggctttagttaaccttttacctcttatatactctagcaccgccccaagatcatgagcagctggggcggggcaggagggaggaatggctgggatttcaggtgctaagaccccctgacactctcctccctttctaatacaggtgttaactatttccataccctagccataggcctcaccgtcactgtggcatttggttcattttgttgtcttgattcaggtcaaagtattctggaggccaccatagcaatgtctgttgcttgatgagcatggtcaaggcaggaggggactgcagagagggagtggcacatagggatggcatgtgaagaccgagtggcagacttggtgccaaagcctgccagggacaagtgtttgtcccctaaactacctgtgacatgaaggaaggaactgagccaggctaggaagttctcccgtgaccagcccaccccagagctccagcccttcctgcagtttccttggtgctgtgctgtgagaggggtgcaggttggtgagaagctctccagctgccaggcttgtgggtgcttctagcagagtgcaaggtctccagtcacatccttggctggggacggcatctgaggacttgggccttcattgcagcatcttcagacaggcggggaggagggaggaggtcttggccttggacggctgttcttcctcagtggcatccaggaactgctgcatataactggaggagccaagcagcatgtggcctgtggcctgcatgctgaagagggcccagcggagcatttccctgggagacagcaaggcactcaggttaaacactccccatgctatctccccaaatgtcttctccatttttctatgctggcctaaagcagttggcttcaagttagacttctttctttgtttctacttcttttatttttgagatagaatcttaacactttatctcaggttagctagaaattcatttatgtagctcagtctggctttgaactcacagaaatctcctgcctcaatctcctgatgctgttgcagttatgggccaccacacctaactttttttggtttggtttggttttgatttggctctttttgaaacagaatctggagatcctcctgtcttgtcctcccaagtgctaggtatacagacatgtcccaccatactgggctcaagctagcttttctccttaggaagatttaagtgtcactaaagtaaaatgacaaaaatgcttccaagtacagacaggaactgggaaccaggctcatagctagccatcccttacacatagttctgcctctactcttgtgacaacttttctggaaccccttgctttttcccatgcttaacttgagttctcaaaacaaagctttttattttcttgtaatgatattttgtttctttcttttctcttctatggtgctggggatggaacccagggctttatttgtgcataagcagctgacctgccatggagctatgtccccagccccatatattttttttttcaagacagggtttctctgtgtagctttggagcctatcctggcactcactctggagactaggctggcctcaaactcacagagatccgcctgcctctgcctcccgagtgctgggattaaaggcgtgtgccaccaacgctcggcatccagccccatattttttaaactactctggaccagccagattaatttacataacacatgtctaccctttgtaacactgcttcttagactttatagtcttcccaggccagttctcagaatgctggccacaaggctcccatgcctgatatcatattctaagcatagaacttggaccagacagggctgaacactgatccagagtggtttgtttatatctaaaatatttaaaatatattttaaaatattataaagatgggtgatgtaactaatttagtgcttgcctttcatgaacaaagccctgggtttgatccccagcaccgcaataacccagagtggtaatataggactgtaaacctaggatccagcactgtggaggtagaagcaggtggatcccaagttcaaagtcatccttggctacatagcaagtgtgagaccagcctgagatacatgagaccctgccaaaaaaaaaaaaaaaaagctttaactgttctggttctgagctccagcagccagaagctttgtgacttgtaaaatgggtaatgagtttgaaactaatgtgaagcacttagcatttggtctgatggacagaaaatgggacatgcagccaagagcagctggctgaatgcaacttctcccacagctattgacaagggctcggcaggggtcactaggagttgctggtcttaagaacaataaggaagagaaaacacaagaaagagaaatactgccaggccgcggtggcacatgcctttaatcccagcactcaggaagtagagacaggcaaatctctgtgagttcgaggccagcctagtcttcagagcaagttccaggacagctacggttacatagtgataccctgtctcaaaaaagcatatatatatatatatatatatatatatatatatatatattgaatttagtggcctgaggcaatggctcagtgggtaaagtgcttgccatatggccatgaggccctgaaagcccatgtaaaactggggatggctgcctgtatctgtgaccccagtactcctctcatggtgagacagagagacaagagactcctctgaagctttcaggccagcaagcctggcccagacaacagacaaacagcaaagaccctgcttgaaacacagtggaagatgagaccagcaccttaggctgtcctctgacttcgaaatgcacgctgtggtacatgggtgcccacattcacacacatatagagataaagactggctgggcagtggtgacacacgcctttaatgccagcacccttgaggcagaggcagttagatctctgagtccaaggccagcctggtctacagagtgagtttcaagacagccagggcaacacagagaaataagaaaagagaaaaaaaaaagatataaagactgaaactgaaaatataatatattagggctagaagtatagcttaatggcatggtgcttgtctagcatgtatgaaggtcctggtttaatccccagctttgaagcatgtgtgtgtgtttgtttctttttgtggtactggggaactggacacaaggccttaggcaagtgctctgccattgaactacagcctcagctttcttttcttttttaaaaatgtttatttattttatgtatatgagtactctatctgcatgtatgactttatgctagaagagggcgtgagatcccactatagatggttgtgagctaccatgtgggtgctgggaattgaactcaggacctcaggaacagcagccagtgctcttaaccgttgagccatctctccagccccctcagctttctttttacccccccacacacaatatctctctaagttgcccagtttagccttgaacttactctgtagcctaggcaagcttctaatttgccatcctcctgtctcaatttcctaggcacctgggagtacaaggccatgtatagctttatatatatgtttcgtgcagtgccttcaaagttatttctcaaattagagaactgagttttgactctagccagccagtcttaaaaaggaatgaaaataaggcctattctacaaatgaatgaaccttgaaaacagaattcttgtggggaaagaatcatgatcccatttctatgaggtctctaggatagatcaatggagcaacagaaagtagagtaaaggtgagcagggtctcgggagagggctgagaaccattatttactgagtacagcttctgctgcctggtgcaaaggttctggaaaccaaggcaatgcttgcacaacatgattagttacttcatgctagacacttcaagcggccacagttgaaaaatgttgaatgtgcagattttagcacgggtgttttttcttgaaggtctcattgtgtagctctagctagaatttggaacttgaaatgtagaccaggctagcctaaaattctcaggagatctacctgcctctgcctcctgaatgctggctgggattaagggagtgagctaccacacctgaccccttagtacatttttttttttaaatcacagtaacaccactaagtcactcagctaaccacagtttgacatcagtttttattttctctaagtttttatttttgttgttgttttattttgtttttcaagacagggtttctctgtgtaacagccctagctaccctggaacttggtctgtaaaccagactggccttgaactcacagagactcacctgcctcagcctcccgagtgctgggaccaaagatatgactcctggcctttttattttctttccatgtgtgggaggtaggggttatgcacctgagtgcagtgcccacagtggtcagaagagggcaacatatctcctagagttgcagttacatgtggttgtgagctggctgaggttggtgccgggaactggacctaggtcctcgggctgagtggttccctttttctgtttttgttttcaatacagggtctgatctggcccaggctgtccttgaactcctgattctacacctccaaagtactggaattatagttacattttcacttacaaaaatattagctgtggaaatagctcaggatatgagtatgtgcttagcatgcgtgctgccctgggtcctatccccagcacacaaaagaaagcacatgactgtcattctttgttagcccccagtgccaacccaggctttgagagagttcagctttgggtagacacaaaggacctctggttagctagtctcccaccctgctgtcccaatctactcacttcacgacgcgcttggcccggtagcagtgcaggtcataggaaagtgtgtatgtgtcatagagggtcgccttcacgttcaggcagccgatgaagtcctgggggttcagcttgtataggtcaaaggttacccgggccacatcaatcttctttgttggcttctgggaaagggatagctgtggtctcgtcttgcgctgcaagacaagtccatgttagctcgcctctgggactccttccacgccctcgacactcacccctgctggaggagactgtcacctgttctgatgggggcttccacttctgccccttctgcaggaccatgaacacagtatctcttgccagggcttggaagtattcttctgtctcaacaatcgtgccgtcttcctccagcacgagggagaagggcttgtctttaagtttcaagatatcctgggcctggaaaagaaggttggtgacatttctgccatccctatggagccacagagttgaggagcaaaggcccgcagtaaagcacttcagctgccagagagagagcaagtgagctgccatccatccgtgcgatggactgactgtcactgtgggggcccactcaagggctgtgtctcagtacttcctactcccaagaaggagtgtagtctaatagaatgggagtcatatgtgaggtcttttttattgagaaataaattacataccatgctgggcaatattaggagtcatatcgaaggccttgggcaagctaggcaaactgcagcattgaactatatccccagccttctttgtactttttgtttggagacaggatcttactaagttgtttaggttggcctggaactcactgtgtagtataggtaggcctcagccttctatttagatcttcttgactcagcctcctaagtaacttggattacaggcctacactaccaggcccagctaaactttattttgttggtggtggttttttgtttcttttgctatgtagccgtggctggccttgaactcacagagatccacctgcctctgtctcccaagtgctggggttaaaggcacgttgttatggaataatcttttggtacactgtgaagttgtgtctttgtcaaggcgctttctgactggtttaataaaagaactgactggccagtagctaggcaggaggtataggcaggaaagcaagacacagaggactctgtgaagaagggcagagtcttgggagtcatgagcaaatgcagagggaagcaagatgaaagaaggtaccactatgatgcagagggtagatagtaaaaggattaatttaagtcatatgagctagctaaacacaactctaagctatcagccaagcatttataactaataatgagtctttgtgtagttatttgagaactggctatcgggatagaaaagtctgtctatagtgtgcatcaccatacctggccactattttaaatttttatttttaattatgcgtatatgtatgtatgtgggtcccctggagcaagtgattgtgagctgcccagtgtgagagctgggaactgaacctctgtcctctgaaagtgcttttaacgactgagccaccactctagccccaacaactcatttgtaatcttgttcctgccaaagctacatggcatgaactgaagaaacaggtcattcaaaactgagggacaggaaataaaataactggcttgccatctttaaaagccagctagaagaatcagcaggtaagggtacttgcagtgcaaactttagcatttgagttcaatacctggagcccacagtagaaggagagaaatgactcccaaatgttgccctgtgacctccatgtgtaccctgtggcatgggcatgccagtgctcacacacacgcttcatagacttacagtaataattttaaatataaaataaaactgtctggatttgcaggacaatcagcagtgctccaacagagtctcaagataagcttgggggggttcttgttatgtagccctagttgggcaggaacccctatatagagaccaggctgacctcaaacatgtggtcctcttgcttcttcatgcatcaccacactcagccttagttttaatttcttttgaggcatacccagcgtatcctggaactcaattatgtagcccaggctagcctcaaacctgagatcttccatatctggtctcccagatacgagccaccatgcctggctcatttctccacgtgtaaatatggggaaggggaatgaatggccctttcagcaagaaaataaccacccacccacggttggtgtgaaagacggtagtgcactgccctccttctgtcacttgaccatgtcacttatcacacacacagctctccaggcggaaatgccaaccctccagtgccaacacctttctccgcttaagaccatgccactcatgcgcacagacttcagggctctccagaaaccgaagcagccggcaggctcaactctcagccaaggcctggagagagcctatgcagggtaccggcagatcccttaccttgcccaggaggtcctccaggctgtgagccatgatgcctttgcgaaccttccgatctgccgtgctaactcgacagggcctcgccctggggacttcccggctgggcttagacaccagctgttgggtcaccactgcagtgctcactgctacatgcctggggacaacagtttgtacagcagatgacctgcctgcggctaccaggtctgcccatgcctgcctgcttgctgagttcaggtgtgacctttccttccaaccaatcatggctgcttcaggcggctttcacaaagtccatcagaagacacagctccgatttggaggggcagggaaatcagcgggaatcttgcttaaatcagactcacttcagtgcctctgagtggagcccaaggactgctaacaagtccccaagctgtgtcagtgtggctggttcttggaccagccacattctcctgtggatggcttctgcttttgtggccttgttcaccttgcctaagaggcagggtgaaatggagctggtgtgtgtatatatgtggggtgtgtgtgtgtgtgtgtgtgtgtgtgtgtgtgtgtgtgtgtgtgtgtgtgtgtgtaaatcggcctgtagaatatgtggggactctgaggtacacagggtagaaagaaaggagcagagagaatttgggtcttggctggaattccaagtttctctctgttacggatttgctaggtgacagcatacccagcctgtgactcagtttcctcatctgttgacttggcatagttgccaccacttcccagagaggagtgaggctcctgggagatcaggaacaaaggcacctggtgcccagcctgaccctttctactccagatgtgatgggaactgtggcagtgacagagagtaagatggggactgtctagactgcaagatcttgggtgctaccctaggcctcctgaatcagatctgtgggggtttattgtaagattttgtctgttttgttttaatgggggtcttgcttatttgtttgttttttgggatagcatctccatatatagccctggtttactggaactaatttcctcatctctgtctctgtctctgtgtctgtgtctgtctctctctctctctctggacctggacctatgcagaccagactcaaactcaccaagatcctcatgccactgctccccaagagccaggattcaaaacatatgccaccatgcctggccatatcagctagaccccacatgactttttttgtcttttaaaatgggatgcctatgtagccctgactgacctgggacttgctgtgcccactacaggctggcctcaaactcagatctgcctgcgtctgcctcctgagtgctgggattgaaagcgtgtaccatcacatccagcccctacatggtttttatacctattaaagttttcaaagttctaatagatggctccccagctttccccttccttcccatacccagctctccttacctggacagtgacttagggtacaggaggctgagagacttcatggcatagtccatccttgtcagctggattgtgttggacctggatcggaggcagtaacctatctggttagagtgggaagggagggaggggtccctctctttctttctttgactcagtggatcccagcccccacacggcaaccctctgccccagtctctcctcctacccccaatttccctgaatccacaccaactacactgagtcagcgtctaccatccccacccattccacagccccagaacaactcactccatgtttctctgccccaagctctggttacactgagctgaggaacccaagtcaaggctgaagaggccctcactgatttgtccctgtccctgggggcagagtccacattatgctggcaggaggtgagtcataccacctcatgctgtgagagacttgaaagtcacctattgtccagaaactccacaggcagtcagaccttggagagcctgctctaaccatgggcccttgagcaataaagtcctcctgaggcttagaactggctaagagaactttctgtgatcttaaagactgtctaggcttgtgttcaccaataccttagctgttaccactgagcccttggcaccataactaatgtgacaaaggagctgacaccgaacttctattgaggcttaactaatttagttaggtaactactgtggtttgtgacgccaggtaccccactggacattacagttgtagtggagtaatcctgggctcatgtgccccctgcctttcctcctatattgctgttctctggttcttgcctgctgagctgcaagtctctgcacaggcagtctcttctacctgcaatgcttgtctcaaactcagagctcaacataagtcacctagaagccccagctcctggctgccaactcaggaaagccttttctggtgccctcttgcctggtaccagattgatccctgtgccctgcccttcc

SEQ ID NO: 5 - DNA, 29980 bp, Cricetulus griseus
  misc_feature (1)..(29980): 3′ of SEQ ID NO2
tgttcatttctgctgcagctctcgggtccccactgtgcctgttttcagcccgtagcacagggacccagtgtgaacaccaggagtgggcaggcatggagaactgcctcctcagtgaaggaaaaccattcttcccttctgataactcgggttctccccggtcaaccttacagagttagagaccctcgtccccacttaatgctacccaagcactcttgtttcaccctgtttctaatacccaccactcacacagctccacaaaacccaccacacctgctctgtggtagccaaataccccagctcttctcctccccctcacctcctagcctcctggcaatactgcgtggccaggcacagcccaggcctcctccattttatttctatctcctgtgaagccactcccttggccccagccccagctggggaaaagagcacagggagtgagtctgaaactctccctagggatttcaagccagactgacttccagaaagccccgaaagggggaagaaggcatgtttcgaaagctctagaaggaagaggtactgcccaggacttggtaccaggcaagagggcacagaaagcaacagacggggcttgtggtctgcgttccacccctcacaccagctgtttgcgctcacccgagttgccttctttcagccctggattttctcctgtgcatgaggaataacgctcctgttgaagccaggaatggtggctcagagctgtaatctcagcacttggtagactgaggcataaaaagggccatgagtttgaagctagcctgggctatagagttaggtcctgtctcaaaaacaaaaatatagtcagacggtggtggcacacacttttgatcccagcactcggaaggcagaggcaggcggatctctgtgagttcaaggccagcctggtctccagagcgagtgccaggataggctccaaagctacacagagaaaccctgtctcgaaaaaacaaaataaataaataaataaaaaagaaatatctttttttttttgagtcaggcaagatacttgagttttattatgtctgtctaaaagcaggtgttaggcctatttgtagtgaagcgtggtgtgctggcgctgcaacacccggtgaggaagcaggaacctgatctcagagcccaagaactgcttgacagcaggccttcggcacttgccctctgaaatctcttccaccttcatcaggttaatggagtgtgcacgggcacagtgcgggcacccgtgtcttggtagcactgtttgacggctccagcagcggtcaggtcccggtattcttggcacatgttgtgtgtgtcatgttgtgagttgtagtgcagccataccgcaaagttcttcacccgcagtggggcctcaggtcgcactgcacagtgtacgatctcctcggatgacttcttcatcttgttcatctgtgcccaaaatcgggacttggccaccacatggttcgtcgcaaagatgtgcatgaggtataggagtggtgtgtggcacttccgggtgggcaagcagcgccccaccaccttgtactcccgaagcgtgcctgaggccttcgtggctgatctgtccttgttggccgccagccacaaaaataaaaggaaatatcttgcctataatgttacaatcccatccagcacttgggaagccaaggcaggaaggcctctgtggatgagaccagcctgggctacagagtgacaacctgccaagagaatggggtgggtaatcccaaatcttgggtatagtgaagtacaggactcaagagacagaggcaggaggatcatctccagttcaagacaagcccgagctataaggcctcaaaaacaaaaaaagaatattagatctatgatttattgctataaacactacaacttaaaaaaaattaaaataaggacagcaagatgactcagtgaatgagggtttgaatttgatccccagtacccacatagtacaaagagagaactgactcccataggttgtctttccgtgaaaactctccctcccttcctccctctctctttctctctctctaaatgaaaatgtttacatcaaagcctctacaaagagcattcatgttacacctagcgttggagagatgtctcagtggtcaagagcgctgacttctcttccagaggatccaggttctattacaagtacccatgtggcaattcacaatcatttataactcagttccaggggatctgactccctcttcaggcctctgtgggccttcatgtgtgtggtgcaaagacttgcacgaaggcaaaacactgtacacataaaataaaaataagagacagaaaagggaatttattcagtgtggccataccagaaaagatgagaggaccagtctcttcaaccctgtttttggagtgcagatggtagctctaggaatttacaggagaagaacaaaaaatgggatgagctggtggtgcaggcctctcagtgcttgagaggcagaggcagaggcaggggaatctctgtgggtacaaggacagcctgactgacctggtggattccgggctaaccgaaactacaaaataagacactacctctaaagttaaaacaaaaaagggaggcaataagtatgcatagctaagcattctattctattctgcccaaaccactctaattgacggctacctccagtctgaagtaagggtcctgcagtagctaggaagaggactacctcccaggctgactgttcctggctctggcaacaaactgaaaaggaaaactggttcagaaggagatcagagcagaggggccaaattagaacgaggattgtgccttccttcaggtttagaacaaccactggagaattttaggtactgctgtggtagtttggaacaagacgaggcaaaaacttattactggcaatctgtaatgtccccataaatcctttttatatctatttttaaaaagatttctatattatatgtacagcattctgtctgcatatatgcctgcaggccagaagagagctccagattttattacagatcgttgtgagccaccatatggttgctgggaattaaactcaggacctctggaagagcagccagtgctcttaacctctgagccatctctccagccctctattttattttttaaaagaattattttatgtgtatgaatatttgcatgtatgtatgtgcaccacattcatgcagtgcctatggaagccagaagaggacgacagattccctgaaaccagagttacagggggctgtgagttgctttatggtgctgagaactgagcttccttcctgtacaagaaaagcacatgctgttaaccactgagctgtccctccagccctccccacccctttgtgtgtgtgtgtacttgtatgtgtatgtatgtggacatgtgagcatgtgtggaagtcagaggatggtttgcaggagttggttctctccttctaccctgtgggtcctagggataaaactcaggctggtagcatgtccctctacctgcaaagccatctcctgggctttgtcccaataaatttcttgctctcccttggtatcttcattcttaaagggaaggacagagctacaggtacaatgggtgaaaattggcctttagttaccagtcttgaagatgagtacttaaatttttttcagaatcctttttttttttttaaatctttttatttatgtatacagtattttgtctgcatgtatgcctccaggccagaagagggcaccagatctcataatggatggttgtgagccaccatgtggttgttgggaattgaactcaggacctctggaagagcagacagtgctcttaacctctgagccatctctccagctcaagaatcctttttttttttttttttttttaaggcagggtttctttgtgtagccttagttgttctggaattaggtttgtagaccaggctggcctgaattcaaagacccacctgactctgcctcccagggtgctgggactaaaggtgtgtgccacgacacctggctaagatgagtgctttttaaatgcctatcacatctacaggcacaactgattgggaagctgattcaaaccttgagatagcaaagggatagaggtgttgatgcaacataaagacagagatgcccagattttctccttcctttagtcactgggcgggactctgcaggtccagtgaggactcactggctctcttgttaaagccttatttagagttaagccaatccttggaagaggcatcattcattcattgaacacttgcagtgtgttaccctgttaattcagcagaaagcaagacagctttaggccacacctggagcagctggaccagagaccaagtggaagcaggaatgctgatgtacacttctcatcctgatcccctggaggctgtcagcctgggccacacagtgcgagatcctgcctcaaaaacgtttttgctacccattggtgattgtacatgtactttgttctagcactcaggaggtagggccagcctggtctacaaaggacagccaatgctacacagagaaaccctgtctagaaaaacaaaacaacaacagaaacttttttgtctgatgtttcatcttgtttcttttctaataatgtactgggcagagaaaaacaaaataatgcagttgggcctggaggcttagacctgggattccagcacttaagagatagaggcaagctcattgctttgagtttaaggtccctctgccctgcatagtgagttgcaggtcagtgtggactatagagtaagaccttgactttcaaaaagcacaaggggccaggcgttggtggctcacagctttaatcccagcactcgggaggcagaggcaggaggatctctgtgagtttgaggccagcctggtctacagagggagttccaggacagcctccaaaacaatacagagaaaccctgtctcaaaaacaaaacaaaacaaaacaaaacaaaaatgtcaaaaagcacaagggccatcaaaatggctcagtgggcaattacaaacttgatgacctgagtccattccttgagacctacataatggaaagaactgactcccctaagttgtcttctgaccttcacacataccgtgtgtgtgtgtgtgtgtgtgtgtgtgtgtgtgtgtgtgtgtgtgtgtgtacatgcacgcgctctcgtgagcaagtacgctcctgatactaaattaataaaaaggattaaaaaagtaaaaccacaaacatgaaccaaaccaatgtggtgtaataacctatgtctggagaactaacaggtttctggacccagagaggtagagaagataatctaatgtcttcaggggaattagggggataaagcatcagagcataaagaaagtttccctcacttgggaagtagtggaggcaggagcatcaggaattcaaggtcatctcatctatatagtaagtaagttcaagactagcctgggttacatgagaccctgtctcaaaaaaaaaaaaaaaaaaaaaaaaaaaaaaaaaaaaaaggaagcatggtgcaacagatttgagattgaaacacaaaaatagctcaactctggggcagacagcagagtcaggggaaagtctaaggaagttctgggaaagggtcaggaggcccctccctgcttcattcacgccccaaacatctattaaacattccatgtgtacttggtgtttctggcctgaggatgactcacagaggctgacacactaagttaatcacaggaagcacagaagaaaaaaaaaaaaaaagaaaaaacaggcctaatgcaatggaatgcctgactgagggggttcttcacagtgctttggagacgaccctttgtgtctcattgaaattcatctctcaagaaacactcctcaaatgtttacacattgtcaggcactttgagctgcagggacacagaggactcagttctggtccctgcctttggaattcacaattgttgctcttccaaagaactggggttcggctctcagcacccacttaaggaggcccatgaccatttgtaacttcagctcaaccgcttccacaagtacccacacacatgtctgtcccccaaacacacctatattcttcatgctaaccttgtgacactatgagcaaaatcaaagacattaagaagccaggttcagtgccctgtgccttagtcctaacatttgagaggctaaatcaaacaggaattgaggccacttggacagcatcaagagaaattatctttgaacatatgaaatgatattatcacagaaatagaattgcaatttaaaccaggtatgtgatcccctctccccccaccaaaagaaaaaaaaatagagggctggagaaatggctcagtggtaaagagcacctgctgttcttgcaaaggatctagattgttttgttttgttgagacagagccacactatgtaactctggctgtcctggaattcactatgtagatcaggctagctggtatgtatgtctgtccacaaatgcatgcctggtgttagaggcttccagaagtgtgtgtcagaccctctggaactggagttataggtggttgtgagctgccatgtgggtgatgtaattgtacttgggccctttgcaagagcaaccagtgcccttaacccctgatacttgcttttaaggaatttgcatttttgttttgaaattacatgttggaaaagttttccacatcagtaagaaatgccatccttactatttcttcctgcagcttctcagaaatattttttaatctttttttttaagattttatttatttatttattatgtacacaacattctgcttcatgtatatctgcacaccagaagagggcaccagatctcattcaaggtggttgtgagccactatgtggttgctgggaattgaactcaggacctctagaagagtagtcagtgctcttaacctctgagctatctctccagcccctcagaaatattttaattcaaaaatctttcttagccagctctcaagccactggattacttcttgcttctgctactgacatagacttcatcttgattcactccatacagcaactagatgtgtgtatataaatgacagatagatcatagatggatggatggatggatagatagatagatagatgatagatagatagataatctcacttgctacctaggctgacctcaaactcatgcctcagtttcctaagtgatgtgattacgggcatacactattatgcctagcataaccatttgtttgccttaaaattttttaagattaattttttattatgtatacagtattgtgcctgcaggccagaagagggaatcagatctcattacagatggttgtcagccaccatgtggttgctggaacttgaactcaggacctctggaagaacagccagtgcttttagcctctgagccatctctccagcccccagcctttaaaatttttaattaattttttttttgtttgtgtatgggtgtttggcctgcatgtaggcctatgcactatatgtgtgtagtactcacctagaccagaagagggtgccagagactctggaattggagtttcagtgcatgatgagctgccaagtgggccttaatccccaaaccccagagggttgaagagggatcagtggctaagggtgcttgttcttgcagaggacccagttttgattcccagagcccacatggtagctcagaacaagaactccggcttcagaggattctgcaccctctctgggcctcatctggtaccaggcatacatctggtatgcagatatatacacgccctgagtggtgacaaacactctgtgagttcaaggctggtctacatagagaattccaggacatagtgagaccctgtctcaaccaaacaacctggatcctttgcaagaagagcaggtgcttttaaacactgggccatctctccagcaccacctcccccctttaatgatacataggtcccacatagcctagttcggcctcttaactcctgacataacacctcatgaattcctcatcctcctgcctctaccttttgagtgagtgacgagattacaaacgttcgccaccatctttgttcccagttggccctcaaggccccagggtctcagttcagatcctgccactatggatacatagcattgaaatggggaaggctgcctctgtgctgcctcagcctttgggtgacgcctcttgcttggcactgcattttcaggagcatgacatctttgctctggaccctgaccccaaggttgtgtggagctgtaagaagtgtaagggtgtctggtattggtggcgcgcgcctttaatcccagcactcgggaggcagaggcagcagaggatctctgtgagttcaaggccagactagactacagagcgagttcaggacagccagggctgttaacacagagaaacactgtcttgaaaagcaaaacaaaataaaaccaacaaaacaacaacaacaacaaagtgttaagcgcgcatgcaaccagccactgggcaattagtcaaagatgccaagtctagatagacatgcagttaagaacttagggtctagagagatggctcggtggttaagagaccagaagagtttgcatcccagcgctcgcacagtagctaacaacagtctatcttacaccctcttccagcctctcctggcacaaggaacacacgtggtgctcatagttacaaaacagacacaacacgcatacacaaagaaataactaatttttaaaatgtcttgtaatcaagtaaaagtgctaactctagggactaaattaattcctcagtcgccctactccaggagcggtaaggctggccagaaagaccaagaacgcacgcgcggaggagaaaccacagagtcggtcctcccgggatagagaggccggaagtgctcgcggagctgcacgccgggtgctggaagcctactgagccccgaggaagggctccgctcggggcttggcgtggtgggtgagccggagggtcggcgtgagcggcctgggctttggttctgaatgatggcgtctcgggcaggcccgcgagcggccggcaccgacggcagcgactttcagcaccgggagcgcgtcgccatgcactaccagatgaggtatgaggtgagccaggagcactgaggccttccccgggaggagcctgcgggtctcgggaagcgacgcgggcgagcctcacggtgccgctcccccagccagctgtcgcgtactaccgggtccccggctccggcgagcgcctcgggtctgtttacaggccgggaagcccagtggcctgccctcgcccgcctcgtgctttgaggggatctggcctgcagaggctcaggggtcgatgctcagcccctctgaatgaccttggagacatcatttttcttttttcaaatcgaggtcccgcagtgtctagagttcaggctggcctggaactcacggccttccctcctcagcctcccgagtatgcgcctggtctgaaaactaacattcttaaagaaaactgcgtgtgtgcgtgtgtgtgtgtgtgtgtgtgtgtgtgtgcgcgcgcgcgcgcgcgtgtgtgtgtttgtgtgtgtgtaagtttgcccacacgagagcaggtgtgctggcagaatgcagtgccagtaaagggtgtgtgtgtgtgtgtgtgtgtgtgtgtgtgtgtgtgtgtgtgtgtgtgtgtgtgtgtgtgtgtgtgtgtgtgtgtgtgtgtgcgcgcgcgcgtgtgtgtgtttgtgtgtaagtttgcccacattcttaaagaaaactgcgtgtgtatgtgtgtgtgtgtgtgtgtgtgtgtgtgtgtgtgtgtgtttgtgtgtgtgtaagtttgcccacacgagagcaggtgtgctggcagaatgcagtgccagtaaagggtgttggatctcttggagctatagttacagacgtgtttaaatccacctgagactagtactgaggaccaaaccccgaagtgattttgtgagatagcggcttggtatggggtccatgctagcctccaggagcggcaggtgctcttaaccactgagccatctctccagtccccaggctgtcttggaacttgctctgtagaccagactagaaatcagagatctgcctgcctctgctgccaagtgcaaggattaaaggtgtacactacagccgccctgttgaccctgtctttaggaagatgtcatgtgtactttacaaagagttttgatgtagtcatgtcttaagctttgttgtcacacaagatgaaattaattaccaccagggaatttttcatcaaattgtgctgtacttaaatatgcctaaagtaaaatacttggggtcagactcttgaagtttaaaccaacaccaggtactgagttgtgttgaacccacacaactaccttgcctcctgcagattgttaactctgctggctgtggtggttgaagagacccccacattggcttagctgttaagagcacttgctgttcttggagagggtctaaactcagttcccagcaccgacatcaggaggctcacaaccaaacacctgcaactccggttccaggggttccgatgccctctggcttcccgacagcacaggcgtgtgcgtggtgcacacacatacacatgagataaatttttaaaaagaaagaaaagccaatctgggtcctgccttcaaggagcttgagtgccagaagtgattcttttctccttacctcccacatttcctataacaaattaattttccccttaaatccactgaattattcaactggttcttggcctctttctttacagaaggatgacaagccctttggacccttctttttcaactttaggcccctgcctaatggatgctcttgttttcatgcgtacctagagcagacaccttgcttgtgacaatatgaaaaggatataattgagaccccgagaagatcaggactaagagatgtgcctccttctcatggaaactatatctggagaaatacagggagcccaaactgtgttccttgttgctaggcctccccctaatggcctttgtttctgtgatacgaagccatctttctaaccacagctcagaattcttgtcatggcttggtgagataatgtcttagtacgttgtccaggccaacctccaatcagagatcctctgcctcagcctcccaggtgccattactgttcttcagtcttgaagtctacacatgcatggtctttgtaactgttactacctctctgtgccttggcagttgatttcctgaaacacaaatactaaaaagcttattcctttgtgcctgcatgtttttgcctctgcgtagaagcccacttccactcgtctcattacctctgctgaaatagctctctcctctagatataaccttctctggctttccatctcatgctaattggttcttccctgggatagcggatatcttcggtttatatgtatttacttgagttttatacttcatgtttcccagcctctatcagaggctgtgtcattcagattgtatctatgtgtccagtaccggtgcctggtggtcggagcaatttaaatgaaaattgcttctccctctgctctgccttacagtgtgacgctcaagagtgaaatcaagaagctgatctacgtacatctggtcatatggctgctgttggttgccaagatgtgtgtgggacacctgaggctcttgtcacatgaccaagtggctatgccctatcagtgggaatatccatatttattgagcattgtgccctctctcttgggccttctctccttccctcgaaacaacattagctacctggtgctctccatgatcagcatggggctcttctccatcgctcccctcatttatggcagcatggagatgttccctgccgcccagcaactctaccgccatggcaaggcctaccgcttcctgtttggtttctccgctgtctccgtcatgtacctagtgttgatactggcagtccaagttcatgcctggcaactgtactacagtaagaaactcttagactcttggttcaccagcacacaggagaagaaacgtaaatgaagcctgcctgatggacacatgaagggagctgttcagaatctccatggactgtggcatctgtgatgttggcacctagtgcacactatcctcagattttggccttgagttctctgttaccatctgctgagatgacaaatctgtagtgtttaatttattcttgactagccacaaaccggatgaccgatgtctgtggaacacttcaagttgaggccctccagactgagccttatccttgccttctcttggtcaaattccttacttccatttatcacctcttcatcaccgatactaaaagagatctggaataaatcagtgcagaaattctacttcaatctgtaggtcatggggcaagcaacatttgggaagttgctcccctaaaggctactctgtttactgcaaaccattttaattaaaaaagaacttcaataaagttaagactgtcctgtgctttgtgtttgagatttgattgaatttcaaagttgtattgctctagaggacttagacattatgagaagaatagatgtttcctgagaacatgggactggcgctggaggagagacagtgtgaagagtcttaagtcagctgcagccctctagctcttaggtgagaacaaccctgggatgggggttgctggaggaatggctcaggggttaagagcactgacagaggacacaggtttagttctcagcacccaaatggtgacttaaaaccatttgtaactactgtcccagggcatttgacgccttcttgcctccggatgcactaggcagacatgaggtacaaaaacaaacatgcaagcaaaacactcaaaaaattaaaaaaaaaaaacatggtctctcaatgtagctgtggatgtactagaactcactaagtagagcaatctgggctcaaactcacagagatcctcctgcctttgcctaccaagtacagggatttaaggtatgtgccaccacacttagcaatatatatttggccagacatttaatctccacccataaagcagcttatttggcttggattatgagctcccctttttatgcttgtttgagacagggcttctctatagctctggctgtcctggaacttgcttcgtggaccagactggcatcaaactcaaatccacctgcctctgcctcccacgtgctggcattaaaggtgtgtgcatcactgcccagctgggttacgagctttctatttggttatttatgtttattagacttttatgtgtctaaaactgagtaataatacccaaaaagaatctgcttgttggggctataattttccagtaactgacaaataaacctgagtcaaaataagaagaaaggggtttgagttctttttcccttttagtttctgtcattgattattccttggcctgcttgtttggggaatgtgcaaaggaagctgctaactctatggaagccagaaaaagaaaaaggtgagattccagtgtcgccactaggggcagactgccagtgacgtaacttcttcactcagcccctcccaaagcttcctccacctccaaaaatgtcaaggactggtaactagtacatgggcctttgagaaaagatagcagggagcttccagtcttgggggaatgtgcagtgaacataacatctaattgtgtgagagacccacagctgagatacagtttaagagtcacacctagaagtcttcacagtaaataaggcagcaaaggtgtgcatccctttgcactggactttattatgcctagatgatgctcagatctgtaactgcagagatctggccactacagttgtaattcttttgtcctggaaatgtgttgcttgtacagtctgcaaagctaattcctccagctatcccactgatcatttctttggagcaggggcagtttccagacagaatttctctgtgtagtcctcgctatcctggaactcagagatctgcctgctgtgtgctgggactaaaggcatgtaccaccatgcccagctcctactgatcatttcaaagataacttcttgtgcctcaggcctattgctgttcctgcttcctgtgtttacgggaacacagctctggatcatccaccctgcaagtttaagtccgtccccactccatatacacactgcctattcggtgaacctcctatagataccctgataagttccctagcttctcagtttaacacttagaactctccctacttaaggggaggcctctcagtggtgaagtgctcctctagcatgcaccgaggcctgggtgtgactccctcgtaacaacaaataaaaacaaaccttttctgctcttacattcctttattaatctttaagatgccacttgtatgaaaattcctcaattttcaattcctcaatttgaagaacaactttaatttgtctggttgctttcaccttattcctttattattattttctaacaagacagagcccagaatgaccttaagctatgtggctgaggatgactttgaactcctgatgctttgtctctacttcctaagtgctaggattacaagtatgcaccatcactcctggcttatattactcgtcccccacccccaccccatagaccaagccttgacctttatttttaaaagctgcatatttattctgctttctgacttagtgtttgtgccttcaacactttcacaacccttttctcttcctcaataaggaaagcctgcttgatcctgtcacggacacacttggcacacaaggagccaccacagcccctgctgacgtgcttctttgtctagacagtctcataaggactttgggtctcacagcatgaaccccttcaagtctgcctgggcacacaccacatggggattcaggtgctttcccagtcttcttggtataaaggtaaacaatcctgttgccaggggttcgagacagcctagtttcgttagaggctgtattgtaggaaagcctacaatggtatgtcaaacactggaccattcgagtgcctctaagcaccatccctggaagaggaagagctgtattatttgttttatattgacagaattgctactttgtatgtcttctgtagctcattagtgttcactctgagcttattgaatgaattaacttgcccttttgaggacaaaggtctgtatttcacagaaaacagaactggactgaagcagaaagaagcccatggacaagggagtttgtcatgagcttagctgaggcacacattgctaacttcaaacaactgtgatgcaaacaattaaaactgcagtgcagacccccctgctttctcccaataaattttccacataagttctgtcccccccccaaaaaaaaaacctgttagcacattattagtcaaagagctaaaacctgtttttggtcatcagaatagtgttttactgaaagtctttgaataccttgtagactaatttcactcattgtgaaattagtcaacagttttaaaacactatcatgccaacatcagtttttgttagttttgtgcttggaattctgcttttctagccttttcctataacttttttctgcctcatggttatggtgttcacaagtcctcttgatttagtgggaagaatgctatttgggttagaaagttcagctgggtatgggggtacatgtctaatcccaacctttggcaggttgaggcagcaggaatactctgagtttgaggccagcctgacatacatacatacatacatacatacatacatacatacatacatacaaacactatctcaaaaaacaaagttggagttaaagtatcaaccacaggactaagatcaacctgcctgttcacaggttgctttaaagctccaatagccggttggagagatggctcagaggttaagagcactggctgctcttccagaggtcctgagttcaattcccagcaaccacatagtggctcacagccatccattatgaggtcaggtgccctcttctggtgtgcagatgtacatggaagcagaatgttgttacataataaatacataaaatcttaaaaaaaaaaaactccaatagccaaagtattatgaaggcattctaaaatttttaatttattttcattttatttttggagacacagtttctctgtgtaaaaaccccaactgtcctggaactcactctagaccagactggcctccaactcacagagataaacctacctctgcctcccaagtactgggattaggccgggcagtggtatcgcccacctttaatcccaacactcgagaggcagaggttggtggatctctgtgagtttgagaccagcctggtctgcaagagctagttctaggacagcctccaaagccacagagaaaccctgtcttgaaaaaaacaaaaaacaaacaaaaagcaagttaatctggaattaaaagtgtatgccaccacacctgatgtaaatttaaaattttttttaaattaatttttctatacacaggtgttttgccagcatgtatgtttgtatagcacatgtgaacctggtgcctaaaaagccaaaagagtgcatctgatcccctgggactggagttacaaatggagtgctgccatgtgggtgctgagaattaaaaccaaatcttctagaagagcagccagtgagtgcccttaactgctgcagcatctttctggctcttgtgaaggcattttatggagtctgtaacaccatatgggtatcaaaagccgtgtggtcacctgatctttgcagcaagacaggagagagaaggtgtttgagagacttgagggatggtctagacaccaaagaaaggtttgtgtgttgtgagcaatgactgattattaggttgagattctagccagggagtttactatctcttgtttgatttattacattttattgccatcctaggaattaaatagggttttacatttgctaagcaagtgttctctcaacatccttatatcccctccccgatttctgacacttactatagctcagcttgtccttgaactcagcaatcctgcctccatagtgccgggattatggatgtttccactcccagctgagcctgttcttcatgtgttgcatgggcattgcagatctctcccattgctgtgaggctgaaataaatgtggatgaactttgtagtacatagtcgtttacatatcaagatgcctttccttggtaaacagtgttatgcttgcctctcaaattgggagtgtcttcctgttttaagaataatcctcttccctcttttcttcctctctcctcctttcccctccttctgacagggtctctttatagaatattcctgcctctctctgccttccaaatgttgtgtaggtgtacatcaccatgcctgcctagttagaatgacgtctcagaatgctggaggttaatttcacattcttagtccacattatcctgactggtatgttcaaggtgccctgacagtaggtacagacccagcagacctcaggaagtgaccattacagcaagatcctgtagctgctactcatagcctgttgggagcccgtgcataggaaagaatggcaagaaaaaatggctgccagagggcgtccccatagtatactctgtcactaagcatgcatgcttcagagcttgccaaaacctcctgagtccctggcttggtccccaaataaaggtcattagagcatgaagtcttggtcaactgattgagactcctttggagagtgcaaggcttctatgtagatgcccctgttgcctcctattctgtctaatttcttactctccctgttgacagctgccaatctcctttctcctaggactgtgcccactcatcactcactgtgataagacccctttccttttctttgttccttcctggtctgcctgcctgctcccccccccacctctcccttttcatggtctcatgaaaactgctctcagactcactatgtagctaaggatgactggagctcccaatcctcctgcccctacttccctaattctgggattacaagtttatgccaccacattccactttctgatttccttccttacatcttaattccactagctttatcacactgctaaagctaaaactttctttgcaaaatgagatccagaaacccacagactctccatagtaggttctgacagggaaacagctgacattgtatggtgttctgccttcccatttctttgctgtgtgtgtttttggacaagtctcggtcttcctgtatttccttccttccttccttccttccttccttctttttccattttttatttgaattataaacacgattgttttacatgttaatcccagttccctctccctcccctcctcccctaccaccatccccaactaaaaccctacctatcacatatcctttctgctccccagggagggtgaggccttccataggggtcctcagggtccgtcatatcctttgggatagggcctaggcccacctccgtgtatcttggctcagggagtatccctctatgtggaatgggctcccaaagtccacacctatgctaaggataagtactgctctactacaagaggctccatggatttctgaggtctcctcactaacacccacattcaggggtctggatcagttccatgctggtttcccagctatcagtctggggaccaagagctccctgttgttcaggtcagctgtttctgtgggtttcaccagcctggtctggacccctttgctcttcattcatccttctctgcaactgtattccagttcagtttagtgtttagctgtgggtgtctgcttctacttcttccagctgctggatgaaggctataggatggcatataagtcagtcatcaatgtcattatcaggggagggcatttaaagcagcctctcctctgttgcttagattgttagttggtgtcatctttgtagctctccaggcatttccctagtgcctgatttctctgtaaacctaaaattttccctctattatggtatctcttatcttgttttcttctattcttcccccaactcaacctttctgctccctcatatactcatcttcccttctcattctcctagctccttcctccccttcccaatttgctcaggagatctggtccctttccccttctccaggggaccatgtatgtctctcttagagtcctccttgttacctagcttctctggctttttttttttttttaagatttatttattatgtgtacagtgttctgcctgcaggtcagaagagggcaccagatcccattacagatggttgtgagccaccatgtggttgctgggaattgaactccggacctttggaagagcagtcagtgctcttaaccgctgagccatctctccagccctttctaaaactggacattagcctagcctcaagggttgtgatgctaacaatacaagctaaagagagcaatgggcatgatccaaagccagatagttcagcaaatattgatttcatcccttcccttttcggaacagactccagccacaccaaatatgtaaaccagcaagacaaaaaacagcaggctatcttttcccactttttgctttgtctatgtttgttggacaatgtcaaactatgtagcccagaccccttctgtaactttccatgtagaccaggctggcctccaactcaaattcccagcaatctacctgtctctgcctctggagttctgggaataaaggtgtgcatcaccatgcctggcctcaataagtccttgtcttatggtcttcttttctcctcctttacttcttatcctcctccctctctcttccctttccctttatctttatttctttattgtcaccgtttcttttctgcttctcttaccatgctttaacaaccatcaggaagcaactaaccaaatgttctcatcttgagagtcaggtcagaagatctaggagtcaagaaagacacgtgtaaaatagagctcacataccaagcattagaaaactcgcagtggcactaaaagatggctcaggggataagaacacttacatggtgactcacaaccatttgtaattccagttgcagacaattcaatatctccttctgatctctaagggcacccagaatgtacatacatacattcaggtaaaacgttcatatacattaaaaataataagccttaagaattagaaaagagggctggagagatggctcagaggttaagagcaccgactgctcttccagaggtcctgagttcaattcccagcaaccacatggtggctcacaaccatctgtaatgagatctggtgccctcttctggtgtgcagatatacatggaagctgaatgttgtatacatattaaataaataaaatcttaaaaaaagagaattagaaaagaaaaaaaagcttcatgtgtgtgtggaaacctccatgaggtagaatgggataaaagctcttgattctatgcttagtctggctagatcacctctcctgttctctggatctctctgacttgactgtagaggttggctctgtacaaggcagcactgtcagcagatcctgttggttcaaagaggtaggtgaagccaagagactacaggtgacagcagattgggagggcccagcacaggtgcagctcactggaagggtccagcacaggtgcagctcactggaagggcccagcacaagcgcagcaccttgggctatagactttgaccttgaccaaatcactttcctctctgcttccttatttctctccttacgaaattaaacaaataatcctcatttacctgcctcacaagttgtttagagggatcaaatattgctgttgctttgtgaactgtaagtagataagctatttagatgagatatatcatgattgacattcactcagccatgaagcacaggcttcctgacaggcactgctaggtgatggagaagaacaaatgagtctctccatctctcttttctcagcactaggattgaacccagggccttgcatacactaagcaagtgcactgccgccgagcttatctgtttttacctccttttaaaaatattgtttatgagctgggtggtggtggcacacttctttaatcccagcacttgagaggcagaggcagggggagctctgtgacttcgaggccagcctggtctgtagagtgagttccaagacagccagggctacaaaataaaaccctgtctcaaaaaacaaaaacaaaaacaaaaacaaaatcaacaaaatgtatgaatgcaaaaacaaaaggatactttagggtcgcaggtagattgggaacttcaaatgtgggccaaaagaaggctgtggccctgcttctgaaggatggtccagaccgaatctcagaaggacaaagtgtcggggctaagatgtagcttcgttgatagagttcatgcatgcctagcacgaaatcctgggttcagtctccagccctgaagaaactggatatggtggttcacgcccgtaacctcagcacttgggaggtagaggtagaaggaccaggagtttaaggaccgggcccagcctacgatacgtgagaacctgtcttgatgatgatgataatgggactggagagatggctcctgtgtaaagtgctgcataaacacgaggacctgagttctggtccccagcaccagcatgataccaggccatggcagaatatacctctagctccagtatcgagtagaggagacaggtggaccctggggcttgcctccccaccagcttagctgagacagcaaggtctaggttcaatgagagatcctgtctcaaaacaaaatagagataaatcaggaagacaccctggtgtcagctctggcctccacacgagcctgcaagcacacctgcccatacaacacacacacacacacacacacacacacacacacacacacacacacacacacacacacacacaccctggtgtcagctctggcctccacacgagcctgcaagcacacctgcccatacaacacacacacacacacacacacacacaaacacacacacacacacacacacacacacacacacctaaatttttttaattaatttaaaaagaagagacaatagcaataccaagatgcaatgacccagtggacatgcccctaactgtcctcccatcgtggagattgccaaggtgaggctgaagcagctaaagtctccatgaatgagaggaagctgtgagatgatggggagggtccccacaaagcctggagccttgtcagccttttttcctgcaccggaaaccacaggaaaccaggtcagtgtgtgtggctgctccctctgctccagagctcagctgtcagttttggggcttcctgagcaagctgtattatttagtttgtccttgaagctagagaccactttgacctggagtcgagaagagttggaaagccagctagctggggagccagagtgtacatggataacacaagaagtttatttgtgttatatgttggcctatttttttgcctaactttaaatgatgtaccaattgcttctttatcttcagcattcagtatagacaagtgccgaatagctgttaattctcaaaatggaagatgtgtaactgtctatgacagatccaagttctagaacatgctaataggcgtgccagatctgcagtgtttgaggagggttaagaaatgggtgcatttccacaacagggtgctggtccctttcctgttacctctccgccacaccaagagccccctgtgacgtgagagcagtaaggctttctaccaaacaggtggacaagtgaattagtgttcaccagtgccctggctcaggagaaggaatgtgacccagcctgctggttaggatagaggaggagcttcagggcagggtggatccagggcagggcttggtgagagcgcctgctggtactgtgtgctcctcacagctccagggccaggccctgctgccctgctgccctcctgcaaacaacaaagccatggtctctgtgccctgatcctgcagaagcttatggggagccccagactgacaacctggttcctgcctctcctcttactgctcttcagcctgtctatgtctgctgaggttggctgtccctacctgccacgctggaccagccactgtctactggcttcccatgtggtaaggtcctgctgctaggtgggggacacttgcttggcacagagtagaaactccccagtctcctgtctgccctttcctacctctgaaatccctgtgatctagtggcagcctctctgtgtccaccatgtggtcctgaaggttctgttgggctgaggtccttggaaggatgagaagaaaacagctggggtggatggacagtgggctgtgggtctcagaaccggatgatcggcacaaagagttcagggagcatgcacctcctgcagttcccttctaaccacacctctctcatttcttttccgcactgggctctgtacctccaggataagaattctgccggtgagtcctgtttcttgttctgcttcactccccatgctcccctgagttccaggtcccagtgagccatccatggccattttggttcccagcctggggagtagtctagtatcaatgggaaagtgcgggactagtgaagatttgcattgtaattctgcctcagacgctatgactgcttttcactctcccatctgaccctcggtctctgagcacaggagaagaggagaaatgattatgcgatgaggacagtgttgctctctaccaatggggaggataaaatgagcaccatggctaatctccaggcactctggacaagctggctcttcttatcaccggtacctcacttgaccaggaacaataatcatctttcatactttattgtgcattctgctttaccaagctttcccccatgccttatgtcacttggtccttatgacaaacccactttgagcagtctggcacctgcccaataacttaaccaagattgtacctgtaagctatatcagagcccagcccgaaatccagtttggagaaggtaggtgggaccccttaggccctaccatttttcttgttcctctttttgtttttgttttgagatagggcctcactatgtaataacaaagcctccataattatttatttacttattttggttcttcaagatagtgtttctctgtgtagtcctggctgacctggaactcactttgtagactaggctggccatgaactcacagagatccacctgcccctgcctcctgagtgctaggatcaaaggcatacgccatcaccacccagctgcctccatcttttaatccttccatctcagcctcccaaggaatagcttacagccatgtgttattgtgccgactttctatcatacacaggaagttctgttcaaactccctgccctaccctcctagccacgaagccttggcactggctctgctgtttgtgcctgcgtctgatgtcacggccttcaggtaagaaaagcagcctttgagccattcttagtgaaggcatgaatcagggacagttgaaaataactagctaggctgacacaagtgtcaattgtcatattgggacatatatttgtcttgttaacctcaagagggacttagagcatcctagaataacacatcatgacagacattcatgggaccactatatctttgtggaggtgccatctttcatctggcactaattcctaaaagccatgtgttctgtagacttgctattgttaccttccctctgtaaaatgggactgccactctgctgcttcattgcctcacttggtacatacggaacacacactttaaaaccagcagtcataggcggatggaaagatagcttagccatcaagagtactagatgctcttgcagaggacctaagttcaattcccagcacccatatcaggcagcatacaactgtaggtaactccaactctaaggaatctgatgtttctgatctcttaagcctcatttatatgtgccaatacatagatatacatacacatgcaagtaattaaacataaaataaaatcacaaaaagttagtttaaaattgttcaccattagctgagtgctagtggcgcacgcctttaatcccagcactcgggaggcagcggcaggaggatctctgtgattttgaggcccgcctgttctatagaatgagttctaggacaggctccaaaacaatacagagaaaccctgtctcaaataaataaataaataaataaataaataaataaataaacaaacaaactaagaaagaaatcatgctacaattaacactgcttttgtgggaggtgtgtgtgtttgtgtgtgtgtgcgtgtctgtcttttttctttgttttattttattttttaaatatcttatgttagaggtggagagatggctcagttgttgagacagttgctgcccttccagaggatctgtattagattcccagtaccctcatagtggctgtatacacaattgtctgtaactgtagttcagggaatccagtgccttcttttgacctcagcaggcaccaggcatgcgcatggtgtacattcatatgtgcaggtaaaaccttcatacacataaaataaaataaataaacctaaatatttaaaaaagaaatgaaatatcttttatttattgtttgacagtctcatacatgcagacaatctatcttggtcaaaaccacccacaattcctccctcccatgcacttggataactcccaatacatcactgctccacattcatgtctcttttaagatcttgtgtgtatgagtgctttgcctgcatgtatatatgtgtattacattcatgtagtactcatggaagcatcaaatccctgtagcactggagttatagatggttgggagccgccatgtgggtgctgggaaccaatcctgaatctaagaacatcaagtaggctgaactgctgatccatcttcacacacacacacacacacacacacacacacacacacacgcacgcacgcacgcacgcacgcacgcacgcacgcacacagagagccatcaaattttgagacagaggcttactgtgtagtcctggctcacctggaacttgcagagtagaccaggcaggtgtcaaaatcaagagatcagcctgcctctgcttctcaatacaagtataaaaccacgccttgctctgtttgttttgtaaattcacggagtccagctatgagtctgcctgctggaatgttgactgatctggttggcttgaccttgtgcagttcctgtgcaggtaaccacagctgcagtgggttcatgaacacgacagtcatgccatgtctagaagacttcaaaacactcttccacatcttctggctctcacattctttctgccctgtcttctgcatttgctgaaccttgaaggggcttaattctgttttttactgaatcataatttttgccaccaaagaagctcaatatcattgctacccttgatttctataaacattctacccaacattgggccacatctccagataggacaggggccaagaatgcccaggtaagaagtgtgtttccctcccacaggccttcagtggaactggtttcctctcttggtgaagaaatctaaaagtcctcctaagtctgaattctactggaggcgtagaaggccagcatcgtcccaggtaccctatcatttacaacactatggccctcagcccctttctttcttgccccttattttgttcttcagcaacaagagggagtttgaaaatgtggagattacgcactaagggaaactgaggccaaaggaatttgaacaacttgcctgttaaattaatttgtctctcagggttcagaggcagagttagaaagcacacttcctggatcctggcagacttcctggatcctggctcacatccactttgttctctgcttgcagaggaagctgctaagcagcctttccctgtctcaggaagaccatagcatttccaaccccttcccagccatctcccatggagggccacactgcaaaaggacccagcctttggacaccggaggagtagaacattttcctagagcaggttcacagaagcgtggaggtaggtgcgaggagcaacaagctggtggtctcagccttgcctgagccagtctctgagtcgtccttctccctaggacctgaattctcctttgatttgctgcctgaggcacaggctattcgggtgactattcctccaggcccagaggctagtgtgcgtctttgttaccagtgggcactggaatgtgaagacatgagcagtccttttgatacccaggtatggagttccatgccttgcaaagagacagattacacaatttctcagcaaatgggatggggtgggggggttggggcccagacaaggaaagtaattgtccaaaacttccttacaaaatgagataagtagcattaaaaacttgttgggggcgggggaggactagatagaaaagatggtgggctatgttacttgagaagagagaccagaaagaaatgagaatggtatgtattgccagggagaggactcttggtgtgattcgggtcaaattaagagactttgctaagggctggggacatacccgttggtagaatgtttacctagcatgcttgatctctggcactgcacagatggagtgggaggtggaagcaggagaatcagaagttcaaagtcttccttagatatataatgagtctgaggtctgtttggagtacgtgagtcttaagagaaaaaaaaaaaaaaagctttgttatttcctcaggaggttgttccctgaaaatttgacaaaactgtcatatgtgtcttggtacttttccctttcataccttttgtctagccctcagaaatgatattccccagtctcacaatccaggtcttgacagcctactttaactgccactcctcacagatgccattgtatgtagctgggcatcacatgcagagactggagtccttcagcttgcttcatgtctacaatggctataccttctaacaataccagcctttagtagaattgatgcttctaatactgcatggcatacagctggaactcaagaaatgtatcgcctcttccctaaccctactgcttgtagagtacctcaagaaaatggccaggatatagaacctatgagagggagaaataagttccatggagtaggaaagtaaggtatattgtccaaggtcccggcttcatcctgccttgtccatagtgtccatgcttttccttaggtgtctctttttctttccctttgtggttttatcaacagaaaattgtgtccgggggccacactgtagacctgccttatgaattcctcctgccctgcatgtgtatagaggtgagcacagggaaatgtggattagccatggctccttgtccaaccgcatctccagtaagtcaacttagaaaggactcgggaaggatggagatgacttggtgatagagcaggccgtacacactaaggagagactccaaaaaaaccgggctactgggatgcttaggaagctggtgctggctaggctatgagccacaccagaggtggaagggtcctaggcaattccacctcagccctgcctggatgctgtcttctctggtccagatttggcttcagctcacagacacagagtagacagccatcatcgctgtgttcttgagagccaaggttgcatatccctctcaaagtcatgttctttccaaccctccatttctccttgtaagtctgaagataaagatgttttggacatttttagggggaagacaggtactaaaagaagggtatggacagagacctgggaaacaagaagctgacagaggagacaaccagtggactggactcgtagggagtccagagtgaggagtcaacacagggtactgggtacttgagaaagtcaacaatggagctcagagagcctaaaattgagtgatgcaacctcgggaattctctgctgctgggaacagttctttggagacaggaggggtcatcccctctcatttgccactccctcctatgctctggaaccccaacacttacacacactccacagtgctgcccattattgtctgacagctcaagctgtttgatgacagtaagccagagccagaatgaagatgttaggaaagccatttctaacaaacactctctatgctccccaggcctcctacctgcaagaggacaccgtgagatgcaaaaagtgtcccttccagagctggcctgaagcctgtgagtgttgtttgtattaatcccgatgcacattcatactccccctgctgtgtgtgtgtgtgtgtgtgtgtgtgtgtgtgtgtgtgtgtgtgtgtgtgtgatggaggaagaccccagatggcaggaggacattttcttcaggagccaggggaagatgccacagctgtcagccaacagatgagctcctgagtgctgaacattatgaatggaaaaggatcgcccagcgagtgtgtgagagggccaactgtagacctaggctcacttctcagttctgccataggttatccccatagcccctcccctttccctcatctgtaaaatcccaaactgtttttgaggcttgaatgaaatattttaggggaaagtggctggtgttgtctggagtgctgtatgtagaatggtgaccttgaggagagaggacacttagacacaagtaacaaagtctaacgtacataggcagggttatcatcaagggtatcagcccaggacaggagaagcccagagacactgttgtgagcagagagggtgtcacaggatggcacttgactgagctcaggaactggacaggaattagaaagaagaagcagggggaagacagactctgtgaggagcctacacagattctggatacaggcgacagtaagggctgttcagtgtggaggcctggatggaaaggtaaagccaaggaagctgggtagaaagggagggagcgtggagtgcttgcctgttgaggacctgtgaagcctggaattgtgctggaacattggtattttctcctggtgtcctggggtggggtggggagatggagttacagctgggctatggcctcactcttgcacctgccacctgcccagatggctcggacttctggaagtcagtacacttcactgactacagccagaacaaccagatggtcatggctgtgacact

SEQ ID NO: 6 - DNA, 750 bp, Artificial Sequence
  Synthetic 5′ homology arm sequence (3085 bp upstream the allele breakpoint)
ataaaagaactgactggccagtagctaggcaggaggtataggcaggaaagcaagacacagaggactctgtgaagaagggcagagtcttgggagtcatgagcaaatgcagagggaagcaagatgaaagaaggtaccactatgatgcagagggtagatagtaaaaggattaatttaagtcatatgagctagctaaacacaactctaagctatcagccaagcatttataactaataatgagtctttgtgtagttatttgagaactggctatcgggatagaaaagtctgtctatagtgtgcatcaccatacctggccactattttaaatttttatttttaattatgcgtatatgtatgtatgtgggtcccctggagcaagtgattgtgagctgcccagtgtgagagctgggaactgaacctctgtcctctgaaagtgcttttaacgactgagccaccactctagccccaacaactcatttgtaatcttgttcctgccaaagctacatggcatgaactgaagaaacaggtcattcaaaactgagggacaggaaataaaataactggcttgccatctttaaaagccagctagaagaatcagcaggtaagggtacttgcagtgcaaactttagcatttgagttcaatacctggagcccacagtagaaggagagaaatgactcccaaatgttgccctgtgacctccatgtgtaccctgtggcatgggcatgccagtgctcacacacacgcttcataga

SEQ ID NO: 7 - DNA, 750 bp, Artificial Sequence
  Synthetic 3′ homology arm sequence (8556 bp downstream the allele breakpoint)
gtagctaacaacagtctatcttacaccctcttccagcctctcctggcacaaggaacacacgtggtgctcatagttacaaaacagacacaacacgcatacacaaagaaataactaatttttaaaatgtcttgtaatcaagtaaaagtgctaactctagggactaaattaattcctcagtcgccctactccaggagcggtaaggctggccagaaagaccaagaacgcacgcgcggaggagaaaccacagagtcggtcctcccgggatagagaggccggaagtgctcgcggagctgcacgccgggtgctggaagcctactgagccccgaggaagggctccgctcggggcttggcgtggtgggtgagccggagggtcggcgtgagcggcctgggctttggttctgaatgatggcgtctcgggcaggcccgcgagcggccggcaccgacggcagcgactttcagcaccgggagcgcgtcgccatgcactaccagatgaggtatgaggtgagccaggagcactgaggccttccccgggaggagcctgcgggtctcgggaagcgacgcgggcgagcctcacggtgccgctcccccagccagctgtcgcgtactaccgggtccccggctccggcgagcgcctcgggtctgtttacaggccgggaagcccagtggcctgccctcgcccgcctcgtgctttgaggggatctggcctgcagaggctcaggggtcgatgctcagcccctctgaatgaccttg

SEQ ID NO: 8 - DNA, 100 bp, Artificial Sequence
  Synthetic Targeted sequence downstream the 5′ homology arm
ccatgtcacttatcacacacacagctctccaggcggaaatgccaaccctccagtgccaacacctttctccgcttaagaccatgccactcatgcgcacaga

SEQ ID NO: 9 - DNA, 100 bp, Artificial Sequence
  Synthetic Targeted sequence upstream the 3′ homology arm
tagcattgaaatggggaaggctgcctctgtgctgcctcagcctttgggtgacgcctcttgcttggcactgcattttcaggagcatgacatctttgctctg

SEQ ID NO: 10 - DNA, 100 bp, Artificial Sequence
  Synthetic Targeted sequence upstream the left junction of the integrated ERV C 109F
ctattgaggcttaactaatttagttaggtaactactgtggtttgtgacgccaggtaccccactggacattacagttgtagtggagtaatcctgggctcat

SEQ ID NO: 11 - DNA, 100 bp, Artificial Sequence
  Synthetic Targeted sequence downstream the right junction of the integrated ERV C 109F
gaactgcctcctcagtgaaggaaaaccattcttcccttctgataactcgggttctccccggtcaaccttacagagttagagaccctcgtccccacttaat

SEQ ID NO: 12 - DNA, 13384 bp, Cricetulus griseus
  misc_feature: 000036F-021-011-25000_Type_C
cttccttccttccttctttttttaaaacagcaaccaaataaaccaacaacaaaaaaataaaaccttaatcaatgataattattttgtaccagtgtgaacaatgagggtgatatggagagcacaggaagttgtaattgggggtgacagggctatgtatcaaatcccatagttgcatcatccttggtactcagtcagtctatttcctgatttggactctggttgccaggcaccaagaagttctgatttacaagttaggtagcttctgtaatggttttagtctcaggataccctatcattgaaagggaaccctgtgttcaaaacctgcataaggatgagctaatttctcatcttaagctaatggcctgatgatttctatgtatcgtggttcagatgatgaaaacacaagttacaggaatttaaatcaaatagttgagaagccccaagccatatatggaagctgaagataagtgagctttgtattccttttgtattattgtggcagaatcgctgacacagattttagagaggaagatttatttcagtttcagatttcagaggagctttaatccatcatgactcggaaggaaatatgggtggggcatatctgtccaccgggcagatgcttaaagctattgcagtgctcatagccactaagccatctcttcaatcctgaggtgattgatagtcttgattttattttattgatgagaaaatcaaatcacaaagcagttaagcagttgctgaagtcacacagcttgggcgtggtcaagctggaatttgaatctagcagtttgcctgcatcttaattgttgtactctgccattctttgtcgtggttcgaattaaaccccacaggagtatcttgcattgggaatgaatactacatttatcttttattttgtcctacaaatatgacccagagctagtcataaatttgtgaatttttatggtaatatgtaggaggggattcagtagaaactgtacattctgtaataaacaaaatgaccttcctggagctgggtttgagccacctgtggccagatgatgctcttagtgtcacgaagtcagtaacatctatgggtctgtgaaccaggtagtctttgttttctcttctagctacaggatccaaggtactatgagacagatctggcacacagagcaataggatgctctgggtgaggtgtgaccctgggttctgaatcctgcttaggatttagtcttagctgccatgggttcttctaagttagagaaactttccattgaagcatccacttcagaggatggaagcatagggaatgggaatgaaagtcaattaacagtgtagctgagtagttcctgtacacagcagtatgttaacagaaagttaaagtgagcagatggagtaaatgaaaacatgcccatcaggtgaagggaccatttcggcagccataacagctgaacacgtgcttgtctgaccttgtcttagtcactaagattcagatgcctgcctcgtgcaaggaaccaatcagaagttagctcgtggtgctatgctttacggctctgggtgtgtttacggacaagcgcacagcaatgatgcacagagcatagcaaccaccctggagggcctatgggccataacaaccagttgaccaatcaacacagggcaaaccctccaagcctggaggcacacaatcctgagcctgtgtgtaccctagacactccccttagctgccctataagatctctatgagacggttcgagctgtctttcctagccatctgccatggtggatggatggaaggcccgagctaacatggggttgaaagacccccgagaggttctttcgtgaagaagacccacacccaggaatcagcgaaaccacgaactttggatgtaagaacaagaggatttattaataacacggatatccggggcttagcttctgttttgccaagccccgagcccggaaggagggtccttttataagggaaaacaggcatagtacagaagcgaaaagcatcaaatcagcattttctcaaggtttaatagccaggcgaaatggccttatctcctgccggtccagatggctggccttgggacgagcgatccggcgggggttggccttgcgtggggcggggagtgagggccagcagctgcagagatcaaaaggtcgggaggtgctaaggagggaagtgctaaggagggaaggatgtattcgcgcgccccgccagctgccgaccagagtagaaaggctacagcccgggggggggtctttcatcccccatttctcttgtactgaatggaatctttcattcagaggtctctggatattctggatctatctgctttagctggtggtattgttgagttaaaactaaggtttgtatggcagataatcgttctctaatgaattgagttatcttgttcagaatgcagggaccaagtataaggattaataatagaataatcagaggtcccatgagagtagataccagagtagtgatccaggggacttagcgaaccatccttcgaaccatccttgttgagactcaaatagctgttgcctctgttttagcctttccctaagtttggccatgctgtcctactattcctgtatgatctgcataaaagcagcattcttccttgagggcagcacatagcccccttcctgtaaaaataataaatctaatcctcgtctgttctgcaggaccacctcagagagtgatgttaaggatttctcgagtgcactgactgactcttcaggacctggatgtccgtgtgcatggcttgttgtagaagcttaaaatgattaattccctgtagggcaacggttccggtccctatgccggctgctatgcccccactgttattcctcctaataatagggccacggtaaggatattggctccctcttatatcgggttgatttccctagtacgctgtaaacatattcaggttggtggtatgtgactttgggccaaagttctatcaatatacaaaatcaatggtggtgttgagaacagtggtagagacacagggagtcaacccagtactgcaagcccaataagttccataaggggcaacaaggtatcggctgtcctgagataatggctctacttggttacataattcttgatgtgagggaggatcctgcctatgcatagcccctttcctgagacctcagatattgtgagcttgtgctgcatagcagctccacaactggtaggtgctgaggtctggttggagtagttgcccagtattgccacgccttcataatatgggggccgggaaactaggcataaccagcattcctgagtcttgttggggtctgaaaaatttaaggcttggtaaactccttggattaattgtaataatctatctccggtacctggagggtctctggttaactcgatgtttagggcgtctggggtggggagaagatgacagcatctggggtaggagtgatagctttaggaacggtagggggttcagggacttggaattgggttggggcctctggtccgctattactgtgtttggcccgactgattgttggcttaggggagtaatttgtctgtacagggagaataaggtcaccggatctttccctgctcggtgcagccggagtccccagactttaggactatcccaagtagtcctttttccagcatctgtgaaccttaggatgagagggttgcattttcctccgggggtggcgccttgaaaacctccccctccggcactatcataacagggtccgcaggctctttgcccacatctccatggtcctttccctggtaccctgggatctccctccgtttgagagtgatgaaatcccaagaggaagagggtttccagtaagcctcccctgtggtttcacatccccagcttgcacagtacccttcctgtggccccccgcagtcatgagttggtctatggccaggacaaacataaaaatccttgcttcttgtcctatccttccccagggtggtggcatccgtaccctgggaatggttcctggtcagagggatcccaatcagaccctactaagtcacataggtcgacatagagaggagggaacccatcatgtagagtccctatataagtgctgaggttggctatttccccggtccctagattggctattttccaggtgatattataaaccctgtgggggttagttgctacatgggggtaaacagacaaccaatcaacaggagggtgtacgagagagtcttatctttaaaggattttgagtgcggtgtgcggtccattctgatgtagtggctgaatcggtgggtcgggctggctttacatgtgaagcatgtatccaagctgcaatgccgtctactttgagtgcggtgggagtggtaagcaaaacgatgtagggtcccttccagtggggttcaaggttcttggcctggtgacgccggacccaaacggtgtccccgatctggtaggaatggggatggtgggatggtccctctggtctttataagcttgagcaagggtttccaaatctcccgttgtactaattgtagtgcctgtaaatgagcttggagagaaggggagttagcaaaatctgagacatcttgatcaagaaagttaatgataggagtaggtactccatgcaggatttcaaaaggtgtgagcccatgtggcccaggggtgttgcgagcacgatagagggctagggggagtaggaggacccagtctctactgccagttgcaagcgacaattttgttaaagtctccttgattgttctattcatcctttctacctgtcctgaactttggggtctgtaagcacaatgtaatttccaatcaatccccaacaaggtggccactgactgacttatctgggaggtgaaggcgggcccattatctgttcccagtacctgaggcatcccataacgtggaaagatttcttcaagcaatttcttagtaacgatcttggctgtttcatgtttagtaggaaggcttcaacccatcctgaaaaggtgtctacaaaaactaatagatacttgtatccatatttacctggtttaatctcagtgaaatctatctcccaatgtgttccgggccggtagcctctgacccggttcccggaggaagcttcagtctggatgcgttgactcgggcgcacgcatcacattgttcagttacctgttttaaaatgtcattcttccccaaaaggaaattgaattcctcttctcgttcgaggagctcccgcatcttcttggagctcagatgtgtcatcttatgtaaaaatgtcactaggtacttggttacccggtaggggatgacagtctttccttcataagtccaatccccgtttggttcctttgtggctcccaatttctctagggtttggatgtctctttgttcatagtcccatgagggggagggacggtcgttggtgggctctagagccaggaggctagtggtgtctgtggtcagggccgcctctcgggcagccaagtctgcccttcgattgcccctggcttcaagagaatcccctttttgatgtccggggcaatgtatgatacttaaggcggagggcagatgaagagctcttaggagagcgaggatttcctccttattcttaatgtctttcccttcggaggtaagcagccccctccgtctgtaaatttctccatgtatatgggcagtggcaaaggcataacgactgtctgtgtacacggtgagcctcttaccttctgccaattttagagcctgcgtgagggcaatgagctctgccctctgcgcagaggttccgggagggagtgcctgagcccagacgacctgattctctgtggtgactgctgcgcccgcccttcgttttccttgatgcaaaaagctgcttccatccgtgaaccagatgtggtccgggctgggtagaggctggtcagtcaggtcgggtctcgccccatgggcctcagccagtacctgtaagcaattatgagcctctggctcccctgggagagggagcaaggtggctggattcagagtcaccagaggcccaaagtggacccggtctttgtccaacagcatagtctggtaatgagtcattcgggcattagtgagccatctgtctggaggctgtctgatgactgcctcaaccgcatgaggggcatgtatggtcaaaggctgccccaaggttagcttgtaagaatcttttactagcagggctatggcggcaatcattcggagacagggtggccatcccgatgctacaggatccaatttcttggagaggtatgcagtgggccttctccaaggccccagtttttgggtgaggactcccttggcatagcctcctttctcatctataaagagttcaaatggcttagctaagtccggtagacccagggctggtgattcaaggagagcctttttgatgttggtgaaggctttcttttggggctcttcccatttaaaagcaacccctggccgagtgaggggtagaggggagccgccatttcggcaaacccggggatccataggcggcagaagcctgccgttcccaagaactctctcagttggcggggattttgtggggtgggaatgtctaagatggctcgcatacgggcttccgttagccatcgccgtccatcctttatcttgtatcctaaataggtcacctgtttctgacatatctgggccttcttggcggatgcccggtatcctaggctcccaagagtctggaggagggctcgagtgccttccttgcattcttcttggttttggctgccaggaggatgtcatctacatactgtaagaggattagcgcggggtaccggacccggaatcctgccaggtcctgatgtaaagcttcatcaaaaagggtggggctgttcttaaacccttgaggtaacctagtccaagttagctgcccagagattccaaggtcagcatcctgccattcaaaggcaaatataggttggctggtgggatgcaggcggaggcagaagaaggcatctttaagatctaagactgtataccaggtatagttgggtggcaatccgctcagcaaattataagggttggggacagtagggtgtatgtcctctatccttttattgacctccctcagatcctgtactggcctatagtcattggttcctggcttcctgacgggcaacaaaggtgtattccaaggagatttacagggcaccagaatcccttgttcaagtaaccttttaatatgtggccgaattccttcgcgggcttctttaggcattggatactgctgacggatgcaggaaggatggcagcttttaaggttactataattggggcctggttgatggcaaggcccattcctcctgtttcagcccaggcttggggaaatccgcaagccagtgatcaagggtttcctgattgttcgattagtctcttttcatggagtctgtattcatcttctatggacattgtcaagatggtaaggggaattcctctggccctgtgactttgacttctgactctcaaagtgtatttgagcttttaatttggtcaataggtcccgtcctagtaagggtaggggcaatctggcacatggaggaaagatgcataccttaccggtcgcgagctggagctgccgattgtagtccaatggtactgctttccgcctgtagctccctgtacccatgcagtccggtgactcaggggtccgggtgacgattcagaacggaatgttgtgcccctgtatctactaggaaggtgaccggatgccccccgacttgcagtgttatcctgggctcagggggggctcctggccctgacttctctaatcttctaggtttagaaggtcagaggttcttggccgtggaggcttggcccgggggttcttagggcattcttttgcccaatgtcctttttctttgcagtaagcgcattgatccctgtccaggtggggcccccttctgccatccctctgcctattctgtctctgacctgtcactactgtggccaatagcctgctcatctctctattccgcttacggtctcttgcagcctctttctcctctgcttcttttctcagtctttcctctcttcctgggcttcctttctccaacgttcttctctttctgtctgtgtctctctcttattaaaaatacgttctgcttcctttaacaagtcttggagcgtatatccttgtaaattttctagcctttggagcttgtttcttatgtccgggctgactgccaaataaaggacatagaaacgctggtggcctgccctggatcttccggatcatagggagtatacattctatatgctccttttagtctctctagaaaagtggcgggcgattcctccgccccctgtattacctgctttacctgggccaaattggtggtcggcgtcctgcccccggagacccgctaccagcaactggcgatagagacgcagatgggtcctacctgcttcagtagtaaaatcccattcaggtctttcaagagggcaagccgcatcgatttcattgggcagctgggtgggctgcccgttggctcctgggacattctttcgtgcctctagtagaaccgctgtttctcctccgaggttaagagaacctgcaaaatctgttcacaatcatcccaggtaggttggtgagttgtgagaccgattctatgagagatgtcagcctcacagggtctgcagagaaggaaggattattatttttccagttatagaggtccgatgctgagaatggccaatattgaggttggccattcggtccagttcggaggggcaaagcggttgaatctggagcgggctgctccctacgacctcttagtcggtgagccattggggaagggtcaggtgggggagctgaagccgtctctgccgagacggcgggggccgcttctgcctctggcggggcggcagcgccgggtatggcgggggtctcagtcaataggtcagccaacaggttttctcccggggaagcacctcaggcgtcttctttcttcagctttagcgtcctttaccgctgtagggtaaaggctggagtggggagggagctggggattggaaaggctagggttgagggaatgggccggttgggcggttagagggaggaagggaggggagcccttggggtgtaggaaaggacgtatccaggggggggtcctgaaccaaggcctcccaagtgactatgtatgccacctggtcgggatgcccctgtggtccaggatccatagtcttctctttcacctgaaatatagtttggaggttaaaggtaccatctcgcggccagccaacgtcaaaggctggccattctgaggaacaaagagtaatccatttgcgtttacgcacatcaacagattggttatgagcataatcctgcacatctctccagtgtgagagtgttagggacaaaggagtggtgatagtttgtcccatagtttctagacttagtagaacacagataatggttatatacaaagactaagcaaaacagaacaggctttcgctgcgcggtttcgacacagagtcgatagtaagatttgagaggggttgaagaaggggccagccttcttcgtccccggtaaggattgcaaatccttcaagccgaggaccctctccaaagaggttgggaagctgtccagtcatagatctcagctcaaaaacaaaagtacaaaggtgagcccactaatgcttcaatcgctaccaggacgtctcctggagccgggtcgggagtccgaacccgtcaaatacaaatgtacgcagctgtgtactaccaacgcgggcgcgcagttcataaaatggtttcagacagaaagacaggaccagacgagacaagacagtggatccacataacacttacctcccaaatgtgggtcggtggtccctgggcggggtctcgatcccggacgagcccccaaatgaaagacccccgagaggtactttcgtgaagaagacccacacccaggaatcagcgaaaccacgaactttggatgcaagaacaagaggatttattaataacacggatatccggggcttagcttctgttttgccaagcccgagccccggaaggagggtccttttataagggaaaacaggcatagtacagaagcgaaaagcatcaaatcagcattttctcaaggtttaatagccaggcgaaatggccttatctcctgccggtccagatggctggccttgggaccgagcgatccggcgggggttggccttgcgtggggcggggagtgaggccagcagctgcagagatcaaaggtcgggaggtgctaaggagggaagtgctaaggagggaaggatgtattcgcgcgccccgccagctgccgaccagagtagaaaggctacagcccgggggggggtctttcaggtagctcgaaacaactattagccttgtgctttctctccagatgccaagctggctaatctgactgagcttccggtctacacaggaaagttctgactgcagacaagccattctagatcagtaatggctggggtgaaggctgacttggccaccaagcctttgaagttgaaggagtttattttcaaagttaacaaaaggaacctcctgctggagcaatttatgaagcttgtttctctggtgtcgaagccatggaatgagaacagtgtgccaagaaaagctttaatgagttcttggagaattaagtcagacatgattactaaaggaagtcagacaggtgaagggaagtttccccagtgagattttacaagttcagtggctttcccatcctcggtgctactgcatgcccatgtctataagccatgatttccaagcaacctttaaggacttcctcttggtgacagcccactgtggcccttcccattctcacatttgggtttcatttgagtaccatgtccccaaatattaaccctcattcacagcccaaggtctcaatgtctttgaaactggtgatcctgcccagctcagcctctccataagtaagactctggtttcatgctgcaactgccacgtggctctggaatgagtatttggcttgtatcccacatggccaaactctcagcaatgcacagaaaagcagagaaggaagcaacaatgcttagacagcttatctgtatcaggctccaaacaagtagtgttatggaaattggttggtagtgtctacaaagaccttgacaagagggtgtcgggatcaaaatacaaaacagttatggaatgtctcaaggtccagcctccggtttcagtaaacggagctccctcccggcccactattgtattgctaatgctagaaggtctgctattgtttaccatagcctcagtatagaatgcctcccagtttgcatctctacaggactaagcatttgaatggccagcacctgggcggaggctaggtgtgtaagtaacatgtgaggacagagagttaggagcgaagggcagagagaggagtcagaaggagagaaacgaccttgtggccatggaagcatggataagaaacagccaagaagatggtaaataaacaagagactctagttttacaacatggaactgagagctctctaaagatgacaagatgcctgtgtttggtctttatcgctgttgggttgctaggagctttcaagtctgccttcccccactcaggccgaacctcatggctgcagtggcttagggctgcgatggccgggacactacaggatggcctattttacattgtacaaataaagaaatggagtctcagagagtttaataacacacccaaagtcacatagctagtgatgggagaggcagcatttttctgatgcagttttgttttcttccattctttccctttcctaacagaacataaaaagttcctctcttctaacctgaactctgggtagtgtttcctagcagggtagaggctgttgtgtaggcagtctctttggtgatgagctttccatcatggagggaagctctgtaagatgttgtatattctacccttcaggaaagtttagtaaaactcaggaagtgaacaactcaatagcttcaggaagttactgaaactgactagattcactaagcccctccccatcaagcatattaatcagtaaggactgctaagagacactctctcacaagataagccacccagaggaagcagagatcagatgaactgaccagaaggggcagaaaccagcctagtgtgggaagagaaagagaccagccagttgaggaatacaaagaggctcaggctatctgagttgcctgggagagatactcgctaagcttttgagttgcctgcaggatgagcagtgtgctccaggtttgcagcttttgtgagatatctcccatgctggtgtttggtgatgcaactgactttgagtcattgctgcttctataagtaaccctcactcatattcctataactgaccccattaaaactcattggtgcaccaagttggactttctgtggtatttttactttggtctgttgtgggttcctatctgggatgagtagatatttgttcccatatccccaggaagagtgtcacacagcaggagccaaacatccacccccaccccatgactttcagaggccctaatcctgaccttgggtgatttatttgatctaagtctcaattttcaacacggataaaatgggaagaatgccaagtattctatcagggtcttgaatgtaagacaagacagcatacaagtgcttcacctcagtcaggaatattctcaatggcagaataatcagtgttaagctgcctattgagatcgctccatcctttctcctttgtggcccactcaggtttcctgggtgagttatggtcccaaagatacccggcctgaactgacaaaaattttattatgcacttaagggtaagtctttggtaatttctttgaatctggtcattgtggggaagcatggagacaaatctagaaagaatgcacaactttccttggtgccaactcaaggaaggaagtattttttagatttaagccctgggcttccagccaaactccaggaataatggaaactgccagggatcgaggggaagttggaagatgagtcactcacttggggtgagtctggcagccagagttccagacatttcaaggtgtctgagagatgaagtggggagaggctagagacaggaagtgcaggtgatgatgggattttatggaaggtctgtgacctcccctcagccctacaaataggccacatgcctcttgagcccacacattccctgtgtcacctttcttcttgtgtgagggtcctcctgcaatctctgctctgaagctctgcaacctatcattagaatgctgctttccctctgactatcttccagacccattgtctgctaccttcaggaatggaggagattgactaatgcccaccttcgcatccctctggagacaaaatcgcttttttcctctgttgctcctcatggccttttttttt

SEQ ID NO: 13 - DNA, 10808 bp, Cricetulus griseus
  misc_feature: 000066F-017-011-39146_Type_C
cccgaagaggcactttcgtagagggagacccacacacccaggaatcagcgaggccacgaactggatgcaaaaacaagaggctttattggagacgcggtaccggggcgacttagcttctgtgtggctaagcgccccgagccaagggaaaggggtccttatataggaaaaaggcgcaagctaggagcaggattctattggataattctaatgaggcattatacagagctattcccattggtcaatgtatatgaggcgctatacagggttattcaaatgaggcaaagtacagagttattcaaatgaggtgaaacacagagtctttcaaatgaggcgaaatacagaatcatttccattggatggttcaaatgagacacagagccattccagatcagcatattctcaaggtctggtgtctggtacagcagactcaattccccccctttcctgatggctgaccttggggcggagaccttgggacggagtgtttctcttcgggcggggcgggggctcaggggcagagctccaggccggctcactcggtgtttgttctcgtgctgacccacctggtgctcgccctcgtgccagcccacccggtgctcgttctcgggctggcccacctggtgctcattctcgggccggccccacccggtgcttgttctcgggccggccccacccggtgcttgttctcgggccgggcggggaaaggccaccggggtggggatcggggaagccgccgacaggaggaagaggagacaaacttgagaaagaaagggctaagggacaggggtctttcattcccccatttctcttgtaactgaatggaatctttcattcagaggtctctagatactctggatctatttgctttagctgatgatattgttgagttaagactaaggcctgtacaacagatagtcgttctctgatgaattgagtcagtttgttcagaatgcagggaccaaatatgaggatcaaaaatagaataaccagaggtcccatgagcgtggatataagggtagtcaaccaggggatttagcgaaccatccctcgaaccatccttgttgagactcaaatagctgttgcctctgttttagcctctccctaagtttggccatgctatccctaactattcctgtatgatctgcataaaagcagcattcttccttgagggcagcacatagcccccttcctgtaaaaataataaatctaatcccgtctgttttgcaggaccacctcagagagtgatgttaaggatttctcgagtgcactgactgactcttctaggacctggatatccgtgtgcatggcttgttgtagaagcttaaaatgattaattccctgtagggcaacggttccggtccctatgccggctgctatgcccccatgttattcctcctaataatagggccacggtaaaggatattggctccctcttatatcgggttgatttctctagtacgctgtaaacatattcaggttggtggtatgtgactttgggccaaagttctatcaatatacaaaagtcaatggtggtgttgagaacagtggtagagacacagggagtcaacccagtactgcaagcccaataagttccataaggggcaacaaggtatcggctgtcctgagataatggctctacttggttacataattcttgatgtgaggaaggaatcctgcctatgcatagcccctttcctgagacctcagatattgtgagcttgtgctgcatagcagctccgcaactggtaggtgctgaggtctggttggagtagttgcccagtattgccacgccttcataatatgggggccgggaaactaggcataaccagcattcctgagtcttgttggggtctgaaaaatttaaggcttggtaaactccttggattaattgtaataatctatctctggtacctggagggtctctggttatctcgatgtttagggcatctggggtgggggagaaggtgacagcacctggtgtaggagtgatagctttaggaacggtagggggtttagggacttgaaaatgggttggggatctctggtccgctattactatgtttggcccgactgattgttggcttaggggagtaatttgtctgtacagggagaataaagtcaccggatctttccctgctcggtgcagcctgagtccccagaccttaggactatcccaagtagttctttttccagcatctgtgaaccttaggatgagagggttgcattttcctccgggggtggcgccttgaaaacctccccctccggcactatcataacaaggtccgcaggctctttgcccacatctccatggtcctttccctgcgtaccctgggatctccctccgtttgagagtgatgaaatcccaagaggaagagggtttccagtaagcctcccctgtggtttcacatccccatcttgcacagtacccttcctgcggccccccgcagccatgagttggtttatggccggggcaaacataaaaatccttgcttcttgttcctatccttccccagggtggtggcatccgtaccctgggaatggttcctggtcagagggatcccaatcagaccctactaagtcacataggtcgacatagagaggagggaacccatcatgtagagtccctatataagtgctgaggttggctatttccccggtccctagattggctattttccaggtgatattataaaccctgtgggggttagttgctacatggggggtaaacagacaaccaatcaacaggagggtgtacgagagagtcttatctttaaaggattttgagtgcggtgtgcggtccattctgatgcagtggctgaatcggtgggtcgggctggctttacatgtgaagcatgtatccaagctgcaatgccgtctaccttgagtgcggtgggagtggtaagcaaaacgatgtagggtcccttccagcggggttcaaggttcttggcctggtgacgccggacccaaacagtgtccccgatctggtaggaatgggggatggtgggatggtccctctggtctttataagcttgagcaaggggtttccagacctcccgttgtactagttggagggcctgtaaatgagcttggagagaaggggagttagcaaaatctgagacatcttgatcaagaaagttaatgataggagtaggtactccatacaggatctcaaagggtgtgagcccatgtggtccaggggtattacgagcgcggtagagtgctagggggagtaggaggacccagtctctagtgccagttgcaagcgacaattttgttaaagtctccttgattgttctattcatcctttctacctgtcctgaactttggggtctataagcacaatgtaatttccaatcaatccccaacaaggtggccactgactgacttacctgggagacgaaggcgggcccattgtctgttcccaatacctgaggcatcccataacggggaaagatttcttcaagcaatttcttagtaacgatcttggctgtttcatgtttagtagggaaggcttcaacccatcctgaaaaggtgtctacaaaaattaatagatacttgtatccatattttcctggtttaatctcagtgaaatctatctcccaatgtgttccgggccggtagcctctgacccggttcccgggaggaagcttcagtctggatgcgttgactcgggcgcacgcatcacattgctcagttacctgttttagaatatcgttcttccccaaaaggaaattgaattcctcttctcgttcgaggagctcccgcatcttcttggagctcagatgtgtcatcttatgtaaaaatgtcactaggtacttggttacccggtaggggatgacagtctttccttcataagtccaatccccgtttggttcctttgcggctcccaatttctctagggtttggatgtctctttgttcatagtcccatgaggggaagggatggtcgttggtgggctctagagccaggaggttagtggtgtctgtggtcagggccgcctctcgggcagccaagtctgcccttcgattgccccttgcttcgaaagaatcccctttttggtgtccagggcaatgtatgatacttaaggcagcgggcagatgaagagcccttaagagagcgaggatttcctccttatttttaatgtctttcccttcggaggtaagcagccccctccgtctgtaaatttctccatgtatatgggcagtggcgaaggcataacgactgtctgtatacacggtgagcctcttaccttctgccaattttagagcctgcgtgagtgctatgagttctgccctctgtgcggaagttccagggggagtgcctgggcccagacgacctgattctctgtggtgactgccgcgcccgcctttcgttctccttgatgcaaaaagctgcttccatccgtgaaccagatgtggtccgggctaggtagaggctggtcagtcaggtcggatctcgctccatgggcctcggccaatacctgtaagcaatcatgagcctcgggctccccagggagggggagcagggtggctgggttcagagtcaccaaaggcccgaagtggacccggtctttgtctaacagcatagtctggtaatgagtcattcgggcattagtaagccatctgtctggaggctgtctgatgactgcctctactgcatgaggggcatgtatggtcaaaggctgccccaaggttagcttgtgagaatcttttaccagcagggctatagcagcaatcattcgaaggcagggtggccatcccgatgccacaggatccaatttcttggagaggtatgcagtgggccttctccaaggccccagtttttgggtgaggactcccttagcatagccctccttctcatctataaaaagttcaaatggcttagctaagtccggtagacccagggctggtgattcaaggagagcctttttgatgtcggtgaaggctttcttttggggctcttcccatttaaaagcaacccctggccgagtgagggggtagaggggagccgccatttcggcaaacccagggatccataggcggcagaagcctgccgttcccaagaattctctcagttggcggggattttgtggggtgggaatgtctaagatggctcgcatacgggcttccgttagccatcgacgtccatcctttatcttgtatcctaaataggtcacctgtttctgacatatctgggccttcttggcggatgcccggtaccctaggctcccaagagtctggaggagggcttgagtgccttccttgcattcccctttggttttggctgccaggagaatgtcatctacatattgtaagaggattagagcggggtacctaacccggaattctgccaggtcctgatgtaaagcttcatcaaaaagggtagggctgttcttaaacccttgaggtagcctagtccaagtcagctgcccagagattccaagggccgcgtcctgccattcaaaggcaaatataggctggctggtgggatgcaggcggaggcagaagaaagcgtctttaagatctaagactgtgtaccaggtatagttaggtggcaatccactcagcaaattgtaagggttggggacagtagggtgtatgtcctctatccttttattgacttctctcaggtcctgcactggccggtagtcattagttcccggcttcctaacgggtagcaagggtgtattccaaggagatttacagggcaccagaatcccttgttcaagtaaccttttaatatgtggccgaattccttcgcgggcttctttaggcattggatactgtctgacggatgcaggaaggatggcagcttttaaggttactataattggggcctggttaatggcaaggcccattcctcctgtttcagcccaggcttggggaaattccgcaagccagtgatcaagggtttcctgattgttcgaattagtcctcttttcatggagtctatattcatcttctatggacattgtcaagatggtaaggggaattccctctggccctgtgactttgacttctgacctctcaaagtgtatttgagcttttaatttggtcaataggtccgtcctagtaaggggtaggggcaatctggcacatggaggaaagaatgcataaccttaccggtcgcgagctggagctgccgatttgtagtccaatggtactgctttccgcctgtagctccctgtacccatgcagtccggtgactcaggggtccgggtgaccgattcagaacggaatgttgtgcccctgtatcgactaggaaggtgaccggatgcccccgacttgcagtgttatcctgggctcagggggggctcctggccccgactcctctaatcttctaggttcaggaggtcagaaacccttggcggtggaagcttggcccggggttcttagggcattctcttgcccaatgtcctttttctttacagtaagcacattggtccctgtccaggtggggcccccttctgtcatccctctgtctattctgtctctggcctgtcactactgtggccaataacctgctcatttctttattccgcttacggtctctcgcaacctctttttcctcagcttcctgtcttagtctttcctctctctcctgagtttccttcctccaacgttcttctctttctgtctgtgtttccctcttattaaaaatacgttctgcttccttcaacaaatcttggagtgtatatccctgtagattttctagcctttgaagcttgtttcttatgtccggggctgattgccaaataaaggacatagaaacgttggtggcctgacctggatcttccggattatagggagtatacatcctgtacgcttctttcaatctctctagaaaagcggcaggtgattcctccgcaccctgtattatctgcttcacctgggccaaattagtgggtctgcgtcctgcccccggagtcccgctaccagcaactggcgatagagacgcaggtgggtcctacctgcttcggtggtaaaatcccattcaggtctttcaagagggcaagccgcatcaatttcattgggtagctgggtgggctgcccgtttactcctgggacattttttcgtgcttctagtagcacgcgctgtttctcttccgaggttaaaggacctgcaaaagctgctgacaatcatcccaggtgggttggtgagtcgtgagtaccgactctatgagagatgtcagcctcacggggtctgcagaaaaagaaggattattatttttccagttatagaggtccgatgctgagaatggccaatactgaggttggccgttcagccagttcggaggggcagagtggttgaatctggaacgggctgctccctacgacctcttagtcgataagccattggtgaagggtcaggggccgcttccgccaaggcagcggcggagtccgcttctgcctctggtgggggcggcagtggcggatatggcgggggctcctccgttaatagatcaaccaactggtcttctcccggagggagtaccttaggtgtcttcttttctttagccttagtgtctttcaccacggtagggtaaaggttggaatgggaagggggttgggaggaatcaaaggagtgggaggtgtagggccgaaggaatgggtcggttggagcggttagagggggaaggagagagggcccttgggatgtaagaaaggacgtacccagggagggggtcctgaaccaaagcctcccaagtgacgatataagccacttgatcgggatgcccgtgtggtccaggatccataatcttctcttttatctggaatatagtctgggggttaaaggtaccatctcgcggccagcctacgtcaaaggtcggccattctgaagaacaaagagtaatccatttgcgtttacgcacattaacagattggttatgagcatattcctgtacatcttccagtggagagtgttagggacaaaggagtggtgacagtttgccccatagtttctagcttaggaggcacaaatgacagctacaagacaaagaccgaataacacagaacagactttcgcagcgcggtttcgacagagagtcgacagaagaattgagagaggtcgaggaaggggcctccttcctcgtccccagtaaagattgcaaatccctcaagccgagggccttctccaaagagactgggaaaatgtccagtcatagatctaagttcaaagtacaaatctctcacctgaggcttccaagttcaaatacaaatccctctcctgagggcttcaaacgctaccaggacgtctcctggagccgcggtcgggagtcgactcgtcagtcctcctcggatccgccaatacaaatgtacacagctgtatactacaaacgcaagcgcaattcacaagacagactcagaccagacaagacaaaacagcggatccgcacaacacttacctcccagacgtgggtcggtggtccctgggcgggtctcatcccggacgagccccaaatgaaagaccccgaagaggtactttcgtagaggagacccacacccaggaatcagcgaggccacgaactggatgcaaaaacaagaggctttattggagacgcggtacctgggcgacttagcttctgtgtggctaagcgcccgagccaagggaaaggggtccttatataggaaaaaggcgcaagctaggagcaggattctattggataattctaatgaggcattatacagagctattcccattggtcaatgtatatgaggcgctatacagggttattcaaatgaggcaaagtacagagttattcaaatgaggtgaaacacagagtctttcaaatgaggcgaaatacagaatcatttcattggatggttcaaatgagacacagagccattccagatcagcatattctcaaggtctggtgtctggtacagcagactcaattcccccctttcctgatggctgaccttggggcggagaccttgggacggagtgtttctcttcgggcgggcggggctcagggcagagctccaggccggctcactcggtgtttgttctcgtgctgacccacctggtgctcgccctcgtgccagcccacccggtgctcgttctcggctggcccacctggtgctcattctcgggccggccccaccggtgcttgttctcgggcggcccaccggtgcttgttctcggccgggcgggaaaggccaccggggtggggatcgggaagccgccgacaggaggaagaggagacaaacttgagaaagaaaggctaagggagagggtctttcactataactgacaaagctcttgaggtgagttctttataaagctctgtttggtggtgtaaggatagtaaagactgctagcatggtttgatactggcaagacctcttgttagatgccattacaatgttgggagcatgtgtgagaggtagagatggtaatatggagagatgtggtgtgtatatcttctggcttcttctctaaacacacatgtattcgatcatggcagttacatactaatgaccttatctaaacctttccacctctcatagacctcagctgtaacacagttccctccttgtaactcctcacaatagtgatttaactctaacatgagtttaggaggtgacaatccatactttccagctgtagtacctgcatatttttcctttggagtggaaaacccagataaaatgaactagggccagtgttgagcatgttatgattttattcatgggtctgttttttttttttttttaaatctctgtgggaacaggtcagctatgtttttgatatatggatcaatcctgacttcctagaaacctgcaggcagggcttgttgttggtgacaggtgaggggtgagcagacagtgactgttggatgagtgtgactgccttgtgcctcagtcagtctggagatacgcctgcctctgtctcccaagtgctgtagtgaaggcatacgtcaccactgcttggctaagcacatatttttaacattgtcaattgaattttgattaatatggtttatttttctggttaaagagggtgtgtgtttgtatatttgaatatgcccacatctcaaaaggacttgaggtaatagctttatggagtgcctgtacaaagcagtaggaatgattgtgtaatgccataattaaagagatttgtgatcctgttttcaggaccaatattcagaagtttagggttttttttagttgattttcttttttttatttaaaaaaattatttttcattttacacatcagctccagtttcccctcccttccttcttgtcttcccccgccccccagtaaggccttggggagtcaacaaagtctggcataccaagttgatataggacaattgttccccctgtatcaaggttgagcaaggtgtcccgccatagggtagcaagttcatgatcttgggataggtcttggtgccattgccagggcctccccaacagttcaagtcacataactatcacccacgttcagagtgtctagtacagtctcatgcaggttctccagctgtcagtccatagtccatgagctcccactagctcggatcagctgcctctgtggttttccccatcatgatcttgaccccctttgttgatatgatccctcctccctctcttcaactgaactctaggagctcagcccagtgcttggctgtggatcactgcatctgcttccatcagttactggatgtagagtcttatgtgacaattaaggtagtcactaatctgattaaagggaaggcctgttcagatacccttccactattgctagcagtcttagctgggtcatccttgtggatcctgagaatttctctagcacaccaggttt

SEQ ID NO: 14 - DNA, 15000 bp, Cricetulus griseus
  misc_feature: 000132F-023-011-15000_Type_C
atgtgtgactagaaagcactatgattacagtttaacacaatactttaaatttatttaatgtaccaggagattttatctatctatctaatttatttatttattcattaagacagtttctctgtgggtgggtcctggctgtcttagaacttgccttgtagaccaggctggcctcaaacccacagagatctgcctgtctctgcttctcacatgctgggattaaaggtgtgtgccaccaatctcaagctcttttaggagatttttattttataactccatgcacagacctcaggcattaactttataaatgactacaccacaccacaatgtcataaggttgtttgcacctatggaatgtttttgcagcacaatctagagtctgcactgcactttatacactacaggggtctctgtgacctgttgcctttccaactgcagggagaagttcaggggaaggggagggtgggtacaatgcaaggactagaatgttggtctctgtgtctggcccagttggccaaaccagttgcttgacaaagatgtgcttttgcagaattgctagtatcttacagctaggccacttaaatgccccttcattaccttatttaagagaaatgagtgattaatagctttggcaatctactgtactttcgtttgctttatcactgagatagtcaacttttggtaaaggcatctaaggccatcagcagaaagcaaacagcaggaaagaacagctccctacaacaaccctaccacaaccggtgtgacacaataaggacagcccattgcacctgctactcctagaatggtcttcaaggtggtctctgccttcaggcctctccatttggtgccgagtcacagccaggaattgctagtgagccactgaagcccctgaagcaggccagataattctgccttcgctgtcccatataatactacacaagaaggaaacaaacacagcaaagaataaacacagtcccctgaaagaccccgaagggtactttcgtagagggagacccacacaccaggaatcagcgaggccacgaactggatgcaaaaaacaagaggctttattggagacgcgggtacccgggcgactcagctttgtgtggctaagcgcccgagccaagggaaaggggtccttatataggaaaaaggcgcaagctaggagcaggattctattggataattctaatgaggcattatacagagctattcccattggtcaatgtatatgaggcgctatacagggttattcaaatgaggcaaagtacagagttattcaaatgaggtgaaacacagagtctttcaaatgaggcgaaatacagaatcatttccattggatggttcaaatgagacacagagccattccagatcagcatattctcaaggtctggtgtctggtacagcagactcaattccccccctttcctgatggctgaccttggggcggagaccttgggacggagtgtttctcttcgggcgggcgggggctcaggggcagagctccaggccggctcactcggtgtttgttctcgtgctgacccacctggtgctcgccctcgtgccgcccacccggtgctcgttctcgggctggcccacctggtgctcattctcgggccggcccacccggtgcttgttctcgggccggcccaccggtgcttgttctcgggccgggcggggaaggccaccggggtggggatcgggaagccgccgacaggaggaagaggagacaaacttgagaaagaaagggctaagggacaggggtctttcattcccccatttctcttgtaactgaatggaatctttcattcagaggtctctagatactctggatctatttgctttagctgatgatattgttgagttaagactaaggcctgtacaacagatagtcgttctctgatgaattgagtcagtttgttcagaatgcagggaccaaatatgaggatcaaaaatagaataaccagaggtcccatgagcgtggatataagggtagtcaaccaggggatttagcgaaccatcctcgaaccatccttgttgagactcaaatagctgttgcctctgttttagcctctccctaagtttggccatgctatccctaactattcctgtatgatctgcataaaagcagcattcttccttgagggcagcacatagcccccttcctgtaaaaataataaatctaatcctcgtctgttctgcaggaccacctcagagagtgatgttaaggatttctcgagtgcactgactgactcttctaggacctggatatccgtgtgcatggcttgttgtagaagcttaaaatgattaattccctgtagggcaacggttccggtccctatgccggctgctatgcccccatgttattcctcctaataatagggccacggtaaaggatattggctccctcttatatcgggttgatttctctagtacgctgtaaacatattcaggttggtggtatgtgactttgggccaaagttctatcaatatacaaaagtcaatggtggtgttgagaacagtggtagagacacagggagtcaacccagtactgcaagcccaataagttccataaggggcaacaaggtatcggctgtcctgagataatggctctacttggttacataattcttgatgtgaggaaggaatcctgcctatgcatagcccctttcctgagacctcagatattgtgagcttgtgctgcatagcagctccgcaactggtaggtgctgaggtctggttggagtagttgcccagtattgccacgccttcataatatgggggccgggaaactaggcataaccagcattcctgagtcttgttggggtctgaaaaatttaaggcttggtaaactccttggattaattgtaataatctatctctggtacctggagggtctctggttatctcgatgtttagggcatctggggtggggagaaggtgacagcacctggtgtaggagtgatagctttaggaacggtagggggtttagggacttgaaaatgggttggggatctctggtccgctattactatgtttggcccgactgattgttggcttagggagtaatttgtctgtacagggagaataaagtcaccggatctttccctgctcggtgcagcctgagtccccagaccttaggactatcccaagtagttctttttccagcatctgtgaaccttaggatgagagggttgcattttcctccggggtggcgccttgaaaacctcccctccggcactatcataacaaggtccgcaggctctttgcccacatctccatggtcctttccctgcgtaccctgggatctccctccgtttgagagtgatgaaatcccaagaggaagagggtttccagtaagcctcccctgtggtttcacatccccatcttgcacagtacccttcctgcggcccccgcagccatgagttggtttatggccggggcaaacataaaaatccttgcttcttgttcctatccttccccagggtggtggcatccgtaccctgggaatggttcctggtcagagggatcccaatcagaccctactaagtcacataggtcgacatagagaggagggaacccatcatgtagagtccctatataagtgctgaggttggctatttccccggtccctagattggctattttccaggtgatattataaaccctgtgggggttagttgctacatggggggtaaacagacaaccaatcaacaggagggtgtacgagagagtcttatctttaaaggattttgagtgcggtgtgcggtccattctgatgcagtggctgaatcggtgggtcgggctggctttacatgtgaagcatgtatccaagctgcaatgccgtctaccttgagtgcggtgggagtggtaagcaaaacgatgtagggtcccttccagcggggttcaaggttcttggcctggtgacgccggacccaaacagtgtccccgatctggtaggaatgggggatggtgggatggtccctctggtctttataagcttgagcaaggggtttccagacctcccgttgtactagttggagggcctgtaaatgagcttggagagaaggggagttagcaaaatctgagacatcttgatcaagaaagttaatgataggagtaggtactccatacaggatctcaaagggtgtgagcccatgtggtccaggggtattacgagcgcggtagagtgctaggggagtaggaggacccagtctctagtgccagttgcaagcgacaattttgttaaagtctccttgattgttctattcatcctttctacctgtcctgaactttggggtctataagcacaatgtaatttccaatcaatccccaacaaggtggccactgactgacttacctgggagacgaaggcgggcccattgtctgttcccaatacctgaggcatcccataacggggaaagatttcttcaagcaatttcttagtaacgatcttggctgtttcatgtttagtagggaaggcttcaacccatcctgaaaaggtgtctacaaaaattaatagatacttgtatccatattttcctggtttaatctcagtgaaatctatctcccaatgtgttccgggccggtagcctctgacccggttcccgggaggaagcttcagtctggatgcgttgactcgggcgcacgcatcacattgctcagttacctgttttagaatatcgttcttccccaaaaggaaattgaattcctcttctcgttcgaggagctcccgcatcttcttggagctcagatgtgtcatcttatgtaaaaatgtcactaggtacttggttacccggtaggggatgacagtctttccttcataagtccaatccccgtttggttcctttgcggctcccaatttctctagggtttggatgtctctttgttcatagtcccatgaggggaagggatggtcgttggtgggctctagagccaggaggttagtggtgtctgtggtcagggccgcctctcgggcagccaagtctgcccttcgattgccccttgcttcgaaagaatcccctttttggtgtccagggcaatgtatgatacttaaggcagcgggcagatgaagagcccttaagagagcgaggatttcctccttatttttaatgtctttcccttcggaggtaagcagccccctccgtctgtaaatttctccatgtatatgggcagtggcgaaggcataacgactgtctgtatacacggtgagcctcttaccttctgccaattttagagcctgcgtgagtgctatgagttctgccctctgtgcggaagttccagggggagtgcctgggcccagacgacctgattctctgtggtgactgccgcgcccgcctttcgttctccttgatgcaaaaagctgcttccatccgtgaaccagatgtggtccgggctaggtagaggctggtcagtcaggtcggatctcgctccatgggcctcggccaatacctgtaagcaatcatgagcctcgggctccccagggaggggagcagggtggctgggttcagagtcaccaaaggcccgaagtggacccggtctttgtctaacagcatagtctggtaatgagtcattcgggcattagtaagccatctgtctggaggctgtctgatgactgcctctactgcatgagggcatgtatggtcaaaggctgccccaaggttagcttgtgagaatcttttaccagcagggctatagcagcaatcattcgaaggcagggtggccatcccgatgccacaggatccaatttcttggagaggtatgcagtgggccttctccaaagagactgggaaaatgtccagtcatagatctaagttcaaagtacaaatctctcaccctgagggcttcccaagttcaaaatacaaatccctcttcctgagggcttccaagttcaaaatacaaatccctcttcctgagggcttcaaacgctaccaggacgtctcctggagccgcggtcgggagttcggacccgtcagtccctcctcggatccgcctacaaatgtacacagctgtatactacaaacgcaagcgcaattcacaagacagactcagaccagaccagacaagacaaaacagcggatccgcacaacacttacctcccagacgtgggtcggtggtccctgggcgggtctcaatcccggaccgagccccaaatgaaagaccccgaagggatactttcgtagagggagacccacacacccaggaatcagcgaggccacgaactggatgcaaaaaacaagaggctttattggagacgcgggcacccggggcgactcagcctttgtgtggctaagcgcgccgagccaagggaaaaggggtccttatataggaaaaaaggcgcaagctaggagcaaggattctattggataattctaatgaggcattatacagagctattcccattggtcaatgtatatgaggcgctatacagggttattcaaatgaggcaaagtacagagttattcaaatgaggtgaaacacagagtctttcaaatgaggcgaaatacagaatcatttccattggatggttcaaatgagacacagagccattccagatcagcatattctcaaggtctggtgtctggtacagcagactcaattccccccttttcctgatggctgaccttgggggcggagaccttgggacggagtgtttctcttcgggcggggcgggggctcaggggcagagctccaggccggctcactcggtgtttgtcctcgtgctgacccacctggtgctcgccctcgtgcctgcccacccggtgctcgttctcgggctggcccacctggtgctcattctcgggccggccccacccggtgcttgttctcgggccggccccacccggtgcttgttctcgggccgggcggggaaaggccaccgggggtggggatcggggaagccgccgacaggaggaagaggagacaaacttgagaaagaaagggctaagggacaggggtctttcacccaacctggttcagagtatgtatgctgccataggtaattccagaattgcttagatcagaccaggctcatgaaaggaagccacagcagagctgaaaggagttctgacacaggaggatggcacttgggaccaaatggaacaatccaggaaaactcacatgtgtgcctgatgtacgcatgaagacaggctcttagcacatgctcagtcttcaagctgtcagcccagctcgtgctcatgatcacaaagtctgtagacatgtctgtccctaagttgggcagatgggtagtgacttactctgcaaatacgcagagccaaacaaacacagcatcagcaacagtcagatcccaagggacctctccattcaccagaactcagctcccctgccagcaggagtccagtgtgtttttatttctgatttcaaaccttgtttggcaccgtcagctaacagagggactacttctccttcctccacaataatgggacatagggaggtggcattcttcctcctcattggccttgcctaacccaacacacctagccacagcttgatacccagagaaacacatcaaaatggaaataatgttgagagctttcctaaaagcatcctgatgcaaacacctgatctgccttcctttctggaacccaggtatctcatcgcattcttgggaagtgggtggtgttttctcagaagcctccaattcccaaggccttggtttctagagttcacattcctgttcttgattcagtgaaggaccctttgcctctctggactctgcctcccatcataaaatttttaattagatttattcatcttattttatgtgtatgtttggctcacctgtaggtatgtgtcaacatgagcgtgtgtgtgtgtgtgtgtgtgtgtgtgtgtgtgtgtgtgtgtgtgtgtgtgtgtctgtgtctgtgtaccatgtggtgctcacagagcttagaagatggcattagatcccctggaactggagttatcaatgggggtgaatccccatggaggtgctgagaactgaacttgggtccatttcaagggcaataaatgctcttaaccactgagccatcttttcacacacagtctgatggattttcaacaggtggactagacgtctccaataggaactatcacagtttgctgcaatgtaccctacccagcaattgcagacacagccccagagagcaataggaagcacatctaaagtctttagtatgtctcccaaagttacaccccaacacacaccaaggcacagatcctccaatgggagtgaggagctctaggcccagggtcacatgctgaaagcagagcatctgtttgggctgtaccgtacttgctcatataataacaccacagattctcacacatcagctcccaagctcaggactgctgagaccagccttgctttatgtggttagccctatgaacactggttttcctcaacttggcatcccagggtacagtgggggctcagtgtgcaaattgcgcaatggagagttcaatctggtcccctctgggtctcagccaactcaggacctggcaccagagcagatgctggtgaatgaatgcctagggaatgactaacacccactctctgagatacttgggctatagcaccgaatttgcagaagacagcagagcccatgggagcccagcagaggcccggcctgctcagcctggcaccaaggccagagcctccaggtcaggtgtggagttttgggggagacatggattcgcagtcatacagtacagatcctggttcaggaccaggaatcagggtctcctgacctagctggtagccagggcatggtggggctctcctatctggaaatggtgacaggacaagggagtaggtcctggtttgcttggcaaggcataggatggatcattggccacaggacatccactttggatcccagtgctgggtcttgtaccccatttttttctgtatatcttaggatagttgtgctttgttgttttttggttttcttcactttggtttaatggtttgctgttttttgttatttcttttattcttttcattgttttaatagtttgctaaggctatatactgtatataatgagtgcccttttaaaacccgcactgaataaaatgaatgtattttatgaataaaagagcaatttctaactttctactctttttggcttttttccttttcgttttgtttttatttttcagattttttatttaaatttcaaacaagcttgtttttcacatcaattccagttctgtctccctccctcctcccctgaccccttcattagctcactatctcttcccctttctgctccccactgagggtgaagccttccatggggatcttcaaagtctgtcctatcatttggagcagggcctaggcccttctccatgtgtcaaggctgagcaagtatccctatatgtggaatgggctccaaagtccatttgtataatagccataaatactgatccactatcagaagtcccatagattaccgagtcctcactgacatctacgttcagggagtttgggtcagtcctatgctggtttcccagctatcagtctggggtccatgagctccccttgttcacgtcaactgtttctgtgggtttcaccagtcctatcttgaccactttgctcatcactcctccctctctgcaactggattccaggaattcagttcagtgtttagctgcgggtgtctgcttctgcttccatcagctgctggataaaggctctagaacgtcatataagacagtctttttttttccttttttaaattatttttacattccaatcccagttcccctccttcctctcctcctgcacccctacaccccttctcacctcccatctgctcctaagggaaggtaaggccccctaggaagtatacaataacttttcacacttaagtctcttctttagtcatgtgaggactcacactcctgtaaacccagaactcacaaacatggatgtatctcagcaagttccatgaagcttggactgcacaatgatttcccagccatgggtctcagggctacatagacagatggcaccttggatatggatgatgtggtctactccatggacacccaatacataattatggttagctagtggctatccataataaaatgtggttattttaaagaccatttgaatggcaccatcttattattttgtttctggttgctatttttatgtgtctaaaatgttaaaagccttattgttctagtgtagattaaaaaagagtacaataggcgctggaataagaaaggcctggactcagccagtaccacctgtacaactgggacaagttatgcctaagagattcatttgcaaaaatggaatcttcactattgacacaaatattttcagatttaaacatcatacagacataacaattaccaaagaatcataaatttatcgttacccaatctctctctctctctctctctctctctctctctctctctctctctctctctctctcctctccagggtttctctgtgtagccctggctgtcctggaactaactctttctgtagaacaggctggtctcaaaatgactcatagaatctgcctgcctctccctcctgagtgctagattaaaggtatgtgccactaccatctggcttatcatgacttcccaatgccaactttcacattcagtttaaacatacctataatggtgagtaatttcaaatgatttaatattatttatgtcaaatataagaagaaagccatacccaatcctcatgtactagtcccttgttacagtatacttctcttgatctgctatgtgacaatatccaagagacatgattctctattaattttttgtttcctgtaggaattcagtaaaggtagaaactgtgagatattgcagttcatcttttaaatggtagatgtatgtagacagaagtttctctcccgcctggtcccacagctgtttagtcccaaataaatacacagaggtttatattaattataaactgtttggctgatggcttaggctttttactggctctctctctctctctctctctctctctctctctctctctctctgtgtgtgtgtgtgtgtgtgtctgctctctctctctcctctctctctgtctctgtctctgtctctgtctctgtctctctctctctctcaattattaacccatttctattaatctgtgtttctccacatggtcttggtttactggtgatgtccagatctccctcaacagttacatggagtctctctgagacttccctctgggttcctcctcctagaattctcctagtctggtagccctgcctatacttcctgcctagctacatcttgactatccattggtcaaaatagttatcacccattaggatggttattggcaagcacctttggaagcagagatgggaagatttctctcagttcaaggacagcatgttctacacagaaaattccagggcagtcaagtcataatgagatctatttaaggagattttctccgtgtatctctgtcactggacctgtttttctccacccactgggtcccacgccacttaatgaaatacccactctgagacttaatatcagttacaattgtttagccaatgactgaggcttcttactggctagctttctcttaattatcaagagtttcttaattattaagggtttctattaatctgtgtatctccatgtgaccttcacttatgagcaataaaatggactaaatagtgtaactataattcttgcttgaaaactgctttgttttatacagttttactatgttaaagttaaaatgttccttttttatttagacagaagaggagataatggggatgtccttctgtatgctgtgaatacatggtttcttttattggtttaataaaatggccagtagccaggccagtaaccaggcaggaagtatatgtggggctaccagactagaagaatgctgggaaaaggaaagcagagggagctgatagggagacaccatgtagccattgaaggagtaacattctggagcattgcgggtaagtcgtggcaatacatagtttattagaaatgggttaataattaaaagagagctagccaataagaagcctaagccattggctaaacaatagaaacttctttgtgcttattttggattaaatagggctgtgggaccatgaggggcagaaactctgtcttcagaatctggtcacaacagataatgataaaaagcattaaattcctggcagagataggaaaagagaagcaaattatcagagcctctttagcctttatgaatgtgattctaagactcacatttatagggttacacaagaaactggaatatatgcattattatctaggtatgatgtcatcagcaagagttgttatcatatatgatgacactaattctcttctagctttgaacttctaattttgaaaaatgcagacacacagaaaacctgggtcgccaactcacaattggatgatattacaaattataaaatattatctctgactcagtctgtgggactttcatttatagacttaaaactcttactcataccttgaagctttgtaaatatccagaacatatatatgccctttggaacactgggtggctggattttaattgtttagtctctaattcttcctataacacactgagaaattgtacatccaacaattcattggaatggttaagtggtcacagttttgatatggccatgagtaattcaagttacaacagtgctgtgtattctctggcccactccctccaagagatgcttcttcagcaatgtgataccctgtcagggaaaattaagcaaagacaacacattgatttgtggaaagtaaagtgtcctgcaacagctgctacttactctcatcctttgtaacactgaatgactgcacagtggtcaatgggagcatgaacaaaatgttctcaagaacattttcattacccaggcagtaaatataggtttggatatacactatcaacaaaaaagattgtcttgacaaaaatttgtgagtttgactaatgattcattgtttgttttagaaatatttatatatgtaacttcataatgtactatggaaaatggccatttgatacaatcctaagtatagaaaaatatcagtggttaacatgctacatagaatagtattgaaaacagtgtttaaaaatcttgcaaatacatgggttattctcttcagatactgaactgtgtttagtgtgtcctgtagcggtcctggtttcaggagggaaaggctgactgctgctttcagggctcccatagtccagagaatgagattctaatgaaacatgagtgaatgcctcaggagacagtcctcacattgattatcctgttcaaaaggctggtagagaaatgaagctattaaatcttttatatctcaacttattcattaaatttgtgtgatcaataattttatctctggatcaatagcatcgccatccttaaactccattttatttaacaattcacaagcatagcagacaatgctttctcaagaaaaaaattcaaatatatttttatttaaatttaccattttcaggctgggtggtggcacatgcctgtaattccagcatttgggaagcagagacaggcagatctatttgcgttcaagccctgcctggtctacagagtgaatgcaggatagctccaaagccaccattcccattcagatcctccccaactccgcagcaggaagaaaactcccatccatttttttctctctctcattcgaatacaaacatgcatattaaaaagcaaaataagataaaaagtaaaaaccaacaaaatcgaataagatgaaacaaactaacagaagacagaagaccaaagaaaaagcacatgaaatacacacagacacagtggcagactcagaaatcctataaaatgtatatcagaaaccatagtatatatgcaaaagatctgagaggaaaagaaaagaagaaaaataaagcccaaacaaattgttatgagaaaagtaacctgcaaaaatgccattgagttcattttttgctggccatctgctgctgggcatggagagtctccttaaaagtggctgttatacccggtaatagtctgtaggagaaaactagcttttcctttgtgagtgtgtttggaagaaatggtccctgacttagggtttctattactgtgaggagacaccatgacgacaccaactcttataaaggaaaacatttgaggtgacagcttacagtttcttaggtttagtccattatcatcatggccagagcatggtggcatataggcagacatggtgccagctacatcttgatcaggaggcagcacgaggtggactgtcactaggctacatcttaggcaaggaagacctcgaagcccaccccacgatgacacacttccttcaacaaggccacacctgctaatagtgccagcccctatgagattatgggggccaatttcactcaaactaccacagccaccaaagggagtggcactaagacacaaagtattttgtcttccttacaaaataaaaaatcctaaatatgtcagataagtaaagaaacaaaaagttgcaatgtttccattaccgtgaagatgaagagttccataaatctgaatacttgatatagagtaagaagggaaaagatggcctaaggtaacatttttatcctatctccaatacacacaaaacaaaaacgaggttactgtgttttggatgatgatgaagacgttgactttcatgcacacttcgtggaagtattttgatcaacagccactctttttaaatgtgctgacaatgcacaccaaactaaaccagtgataatctgaggcaggatagaggatgctctcttggtgaaaatggttgaaagagtgtttgtgccacaccaaatgtcttagttagagtttctgttgctgtgaagagacaccacaagacctgtcctggatattcagggtcactctactggagaaaagtgattatccctctcgcagcaggtataagagacagttcagtttttaaccttcgtagtaatg

SEQ ID NO: 15 - DNA, 7410 bp, Cricetulus griseus
  misc_feature: 000161F-025-011-17947_Type_C
tcggcagagacggcttcagctcccccacctgacccttcccaatggctcaccgactgagaggtcgcagggagcagcccattccagattcaaccactgtgcccctccgaactggaccgaatggccaacctcaatattggccattctcagcatcggacctctataactgtaaaataacaatccttctttctctgcagaccctgtgaggctgacatctctcatagagtcggtcctcacaactcaccaacctacctgggatgattgtgagcagattttgcaggtcctcttaacctcggaggagaagcagcgcattctattagaagcacgaaagaatgtcccaggagccaacgggcagcccacccagctgcccaattaaatcgatgcggcttgccctcttgaaagacctgaatggtattttactactgaagcaggtaggacccatctgcgtctctattgccagttgcttgtagcgggtctccagggggcaggacgctgacccaccaatatggcccaggtaaagcaggtgatacagggggcggaggaattgcccgccacttttctagagagactaaaaggagcatatagaatgtatactccctatgatccggaagatccagggcaggccaccagcgtttctatgtcctttatttggcaatcagctccggacataagaaacaagctccaaaggctagaaaatttacagggatatacgctccaagatttgttaaaggaagcagaacgtatttttaataagagagagacacagacagaaagagaagaacgttggaggaaggaagcccaggaaagagaggaaagactgagaaaagaagcagaggagaaagaggctgcgagagaccgtaagcggaatagagagatgagcaggctattggccacagtagtgacaggtcagagacagaataggcagagggatggcagaagggggccccacctggacagggatcaatgcgcttactgcaaagaaaaaggacattgggcaaaagaatgccctaagaaccccgggccaagcctccacggccaagaacctctgacctcctaaacctagaagattcccccccccgagcccaggataacactgcaagtcggggggcatccagtcaccttcctagtagatacagggcacaacattccgttttgaatcagtcacccggacccctgagtcaccggactgcatgggtacagggggctacaggcggaaagcagtaccgttggaccacagatcggcagctccagctcgcgaccggtaaagtcatgcactctttcctccatgtgcctgattgcccctatcccttactaggacgggacctattgtccaaactaaaagctcaaatacactttgagaaatcaaaagtcaaagtcacagggccagaaggaattccccttaccatcttgacaatgtccatagaagatgaatatagactccatgaaaaagagactaacccaggcaaccaggaaacccttgaccactggctcgcggagtttccccaagcctaggctgagacaggcgggatgggccttgccatcaaccaggccccaattatagtaacttaaaagctgccgcccttcctgcatccgtcaagcagtacccgatgcctaaagaggcccgcgaaggaatccggtcacacattaaaaggttacttgaacaagggattctgacgccctgtaactagtaaaagattcttacaagctaaccttggggcagcctttgaccatacatgcccctcatgcggttgaggcagtcatcagacagcctccagacagatggctcactaatgcccgaatgactcattaccagactatgctattggacaaagaccgggtccactttgggcctctggtgactctgaatccagccaccttgctccctctcccaggggagccagaggctcataattgcttacaggtactggctgaggcccatgggacgagacccgacctgactgaccagcctctacccagcccggaccatatctggttcacggatggaagcagcttcttgcatcaaggagaacggagggctggctcggcagtcacctcagaggatcaagtcgtctgggctcaggcactccctcctggaacctccgcgcagagggcgagctcattgccctcacgcaggctctaaaattggcagaaggtaagaggctcaccgtgtacacagacagtcgttatgccttcgccattgcacacatacatggagaaatttacagacggaggggttgcttacctcagaagggaaggacattaagaataaggaggaaatcctcgctctcctaagagctcttcatctgccctccgccttaagtatcatacattgccccggacatcaaaaaggggattctcttgaagccaggggcaatcgaagggcagacttggctgcccgagaggcggccctgccacagaaaccactaacctcctggtgctagaacccaccaacgatcgtccctccccctcatgggactacgaacaaagagacatccagaccctagagaaactgggagccaccaaggaatcaaatggggattggacttatgaaggaaagactgtcctcccctaccgggtaactaagtacctagtgactttttacataagatgacacatctgagcccaagaagatgcgagagctactcgaacgagaagaggaattcaatttccttttggggaagaatgacattttaaaacaggtaactgaacaatgtgatgcgtgcgcccgagtcaacgcatccagactgaagcttcctcccgggaaccgggtcagaggctaccggcccggaacacattgggagatagattttactgagattaaaccaggtaaatatggatacaagtatctattagttttgtagataccttttcaggatgggttgaagccttccctactaaacatgaaacagccaagatcgttactaaaaattgcttgaagaaatctttccccgttatggatgcctcaggtactgggaacagacaatgggcccgccttcgcctcccagtcagtcagtggccaccttgttggggattgattggaaattacattgtgcttatagaccccaaagttcaggacaggtagaaaggatgaatagaacaataaggagactttaacaaaattgtcgcttgcaactggcacagagactgggtcctcctactccccctagcctctaccgtgctcgcaacacccctgggccacatgggctcacaccttttgaaatcctgcatggagtacctactcctatcattaactttcttgatcaagatgtctcagattttgctaactccccttctctccaagctcatttacaggccctccaatagtacaacgggaggtctggaaaccccttgctcaagcttataaagaccagagggaccatcctaccattcccattcctaccagatcggggacaccgtttgggtccggcgtcaccagaccaagaaccttgaaccccgctggaagggaccctacatcgttttgcttaccactcccaccgcactcaaagtagacagcattgcagcttggatacatgcttctcatgtaaagccagcccgacccaccgattcagcactgcatcagaatggaccgcacaccgcactcaaaatcctttaaagataagactctctcgtacaccctcctgttgattgtttgtctgtttacccccatgtagcaaccaacccccacagggtttataacatcacctggaaagtagaccaaacctcagcacttatatagggactctacacgatggttccctcctctctatgtcgacctatgtgacttagtgggtctgattgggatccctcgaccaggaaccattcccagggtacggatgccatcaccctggggaaggatgagaacaagagaaaggatttttatgtctgcccggtcataaaccaaccgacaatgcggggggcacaggaggatattgtgcaagctggggatgtgaaccacagggatgcttattggaaaccctctcctcttgggacttcattacctcaaaggagggaaatcccgggtacatagagacaggtcacggaatgtggcttaaatctgtggacctgttatgacagtaccaaggggaatattaaaggcgccaccccggaggaaaatgacccctcatctaaggttcacagatgctggaaaaaggactacttgggatagtcctaaagttgggactcggctgtacgagcagggaaagatccggtaccttattctccctgtacagacaaattactccctaagccaacaatcagtcgggccaaacccagtaatacggaccagaggccccaacccaattccaagtccctgaacccctaccgttcctaaagctgtcactcctacccaggtgctgtatcttctcccctcccagacgccctaaacatcaagataaccaggacctccaggtaccggagatagattattacaattaatccaaggagtttaccaagccttaaatttttcagaccccaacaagactcaggaatgctggttatgcctagtttcccggcccccatattatgaaggcgtggcagtactgggcaactactctaaccagacctcagcacctaccagttgtggagctgccatgcagcacaagctcacaatatctgaggtctcaggaaagggctatgcataggcagggttcctccctcacatcaagaattatgtaaccaagtagagccattatctcaggacagccgataccttgttgccccttatggaacttattgggcttgcagtactggtttgactccctgtgtctctaccactgttctcaataccaccattgacttttgtatattgatagaactttggcccaaagtcacatacaccaacctgagtatgtttacagcgtactaggaaatcaacccgatataagagggagccaatatcctttaccgtggccctattattaggagggataacagtgggggcatagcagccggcatagggaccggtacagttgccttacagggaattaatcattttaagcttctacaacaagccatgcacacagatatccaggtcctagaagagtcagtcagtgcactcgagaaatccttaacatcactctctgaggtagtcctgcagaacagacgagggtagatttattattttacaggaagggggactatgtgctgccctcaaggaagaatgctgcttttatgcagaccatacaggaatagttagagacagcatggccaaacttagagaaagacttaaccagaggcaacagctatttgagtctcaacagggatggttcgaaggatggttcgctaagtccccctggttcactactcttatatctactctcatgggacctctgattattctatttttaatccttatgcttggtccctgcattctgaacaagatgactcaattcatcagagaacgactatctgcatacagaccttagtcttaactcaacaataccaccagctaaagcaaatagatccagaatatccagagacctctgaatgaaagattccattcagttacaagagaaatggggaatgaaagaccccgccccttagctctttctcttaagtttgcctcctcctcggtcagcgctggcgggcgcgcggatgcatcctccctgcactcccgatcccgatcccacctcgccgcctacttccccgccgccgcgcaggccagccccgccggccagacgcgagccggacagccagcagcccatgagcgcagacgagccctcctcgcactgtgcaccgactgcccggcgcgctcccccagccggttcaccccggcgggctcgcgccccggcacgccgcctggcaccgtgcccggcagcgagagggcacccttcccccaccgcgcccgcttgatggggaacgctcgacccaaggtcagccatctggacgcggagaagatagtaagccaaggccgtttcgcctgactactagaccttgagaaagtgctgatttgaaataactttgcacctcattaaattgtccaatagaatttctgtaactatgctttcgcttctgtactgtgcttttttctatataaggacccctcttccttggctcggtgcgcttggcctaacagaagctaagcttccccgggtacccgtgtaataaagcctcttgcttttttgcatccaagttcgtggtctcgctgattcctgggtgcgggtctccttctacgaaagtacctcttctggggtctttcaagagagagagagagagagagagagagagagagagagagagagagagagagagaggttcacttatgtggttcaaaatctatttaaagccaggtataattctagcactctgaaggctgccattgaaggagtttccatttgaggctagcctgagctactcagaaagaccttgtctcaaaacaatagcaacatctatgggccggagagatggctcagcagttaagagggcacactgcccttgcgttcccagcactcatgcctggtggttcacaagtgtttgtaactccatctctgaggataccaattctctcttctggactccatgggcacctgcactcacatgcaaatatacccatcctcatatacatataaatacacataataaaaacataaaattgagccaggcggtagtggtgcacacctttaatcctagcattgggaggcagaggcaggtggatctctgagttcgaggtcagtctggtcttcagagagagttccaggacagtcaaggctacacagagaaaccctttcttgaaaaaccaaaaccaaaacaagaaaaaaaccaagtgctgggattaaaggggatgagattctatgcctggcttgaaaacaaatcccacaaaataaacaaacaaaaccccaaataatcccactgagtattacaatgtatttagtgctgctgcacagcaatttaaatgtacttgacaagagtgaatatggctttaataatgtgtggcttttttttttaacaacctaaaaaaatggggagaggaattgagcatcaggggtctcttctcctcttgtcttttacaatacctcgtggactccaccccaatagggttagtcatttctcttagttttgttttttttttgatggatatttcatgttaatttttttatttcaaatgtatgtgagtgtttgcctatatgtatctacatctactatgtgcaggcctgtttccctcatggatcagaagagggtgttggacccctgggactggagttatagatactttccagccaccatgtgtgtgttgggaaccaaacccagactctttgcaagagcagtcagtgtctttaaccacaagacagggtttctctgtgtacctttgaagaccaggctggccttgaactcacagagatccgcctgcctctgcctctcaagtgctggaattaaaggcaagtgccacctgtcctgcgcgatagtcttgccggcaag

SEQ ID NO: 16 - DNA, 13593 bp, Cricetulus griseus
  misc_feature: 000308F-002-011-31438_Type_C
agatgagcttataggcctgcaccaccaggcctacctaaaatagattttgatttaaatgcttgtcttgtttgtttcccagagctgactgtaatcccagttggtgaactgggactgtgacccgctgttgtgaactggcagtgtttgctggtttccccactgtgtttttggttcatctctggctccagggtgatggtcttgagtgcagagcctggaaggaagaattgctcacccactgtggctgcatttccatcatacagtgcagcggactcaatagagcacactgaagagcagcatgtagcaaatgaattgggaatgatgagtataaatatctatttgtttatttggtattttaagacagagtttctctgtgtagccctggttgtcctggatctcactctgtggatcaaactggcctcaactcatagagatcctcatgcttctgcctctggagtactgggatcaaaggcatgtgccatcactactatcttaatactatttttcctaaaaaagagcctcacacatgctaggtctaccactgaaccatattctagccagagacaggttttgataaatttgataaattaattttaattttatttcataatgtatagggaaaatgcagaaaggtctgaatgtaagaaggaagagagaagggtgacagatagtaggacagagaattcgagaggaccaggcaagctgtagcagaagctgaacctgaaagcttgggaccctgctaaatgtggatagtctagaagaggaatctgagtggatctatagatgtggttgaaagacccctgtcccttagccctttctttctcaagtttgtctcctcttcctcctgtcggcggcttccccgatccccacccccggtggcctttccccgcccggcccgagaacaagcaccgggtgggccggcccgagaatgagcaccaggtgggccagcccgagaacgagcaccgggtgggctggcacgagggcgagcaccaggtgggtcagcacgagaacaaacaccgagtgagccggcctggagctctgcccctgagcccccgccccgcccgaagagaaacactccgtcccaaggtctccgcccccaaggtcagccatcaggaaagggggggaattgagtctgctgtaccagacaccagaccttgagaatatgctgatctggaatggctctgtgtctcatttgaaccatccaatggaaatgattctgtatttcgcctcatttgaaagactctgtgtttcacctcatttgaataactctgtactttgcctcatttgaataaccctgtatagcgcctcatatacattgaccaatgggaatagctctgtataatgcctcattagaattatccaatagaatcctgctcctagcttgcgcctttttcctatataaggacccctttcccttggctcgggcgcttagccacacagaagctaagtcgccccggtaccgcgtctccaataaagcctcttgttttttgcatccagttcgtggcctcgctgattcctgggtgtgtgggtctccctctacgaaagtacctcttcggggtctttcatttgggggctcgtccgggattgagacccgcccagggaccaccgacccacgtctgggaggtaagtgttgtgcggatccgctgttttgtcttgtctggtctgagtctgtcttgtgaattgcgcttgcgtttgtagtatacagctgtgtacatttgtaggcggatccgaggaggactgacggtcgactcccgaccgcggctccaggagacgtcctggtagcgtttgaagccctcaggaagagggatttgtattttgaacttgggaagccctcaggagggatttgtatttgaacttggaagccctcagggtgagagatttgtactttgaacttagatctatgactggacattttcccagtctctttggagaaggccctcggcttgagggatttgcaatctttactggggacgaggaaggagggcccccttcctcgactctctctcaattccttctgtcgactctctgtcgaaaccgcgctgcgaaagtctgttctgtgttattcggtctttgtcttgtagctgtcatttgtgccctcctaagcctagaaactatggggcaaactgtcaccactcctttgtccctaacactctcccactggaaagatgtacaggaatatgctcataaccaatctgttaatgtgcgtaaacgcaaatggattactctttgttcttcagaatggccgacctttgacgtaggctggccgcgagatggtacctttaacccccagactatattccagataaaagagaagattatggatcctggaccacacgggcatcccgatcaagtggcttatatcgtcacttgggaggctttggttcaggacccccctccctgggtacgtcctttcttacatcccaagggcccctctctccttccccctctaaccgctccaaccgacccattccttcggcccctacacctcccactcctttgattcctcccaacccccttcccattccaacctttaccctaccgtggtgaaagacactaaggctaaagaaaagaagacacctaaggtactccctccgggagaagaccagttggttgatctattaacggaggagcccccgccatatccgccactgccgccccaccagaggcagaagcggactccgccgctgccttggcggaagcggcccctgacccttcaccaatggcttatcgactaagaggtcgtagggagcagcccgttccagattcaaccacttgcccctccgaactggctgaacggccaacctcagtattggccattctcagcatcggacctctataactggaaaaataataatccttctttttctgcagaccccgtgaggctgacatctctcatagagtcggtactcacgactcaccaacccacctgggatgattgtcagcagcttttgcaggtccttttaacctcggagagaaacagcgcgtgctactagaagcacgaaaaaatgtcccaggagtaaacgggcagcccacccagctacccaatgaaattgatgcggcttgccctcttgaaagacctgaatgggattttaccaccgaagcaggtaggacccacctgcgtctctatcgccagttgctggtagcgggactccggggggcaggacgcagacccactaatttggcccaggtgaagcagataatacagggtgcggaggaatcacctgccgcttttctagagagattgaaagaagcgtacaggatgtatactccctataatccggaagatccaggtcaggccaccaacgtttctatgtcctttatttggcaatcagccccggacataagaaacaagcttcaaaggctagaaaatctacagggatatacactccaagatttgttgaaggaagcagaacgtatttttaataagagggaaacacagacagaaagagaagaacgttggaggaaggaaactcaggagagagaggaaagactaagacaggaagctgaggaaaaagaggttgcgagagaccgtaagcggaataaagaaatgagcaggttattggccacagtagtgacaggccagagacagaatagacagagggatgacagaagggggccccacctggacagggaccaatgtgcttactgtaaagaaaaaggacattgggcaagagaatgccctaagaacccccgggccaagcttccaccgccaagggtttctgacctcctgaacctagaagattagaggagtcgggccaggagccccccctgagcccaggataacactgcaagtcgggggcatccggtcaccttcctagtcgatacaggggcacaacattccgttctgaatcggtcacccggacccctgagtcaccggactgcatgggtacagggagctacaggcggaaagcagtaccattggactacaaatcggcagctccagctcgcgaccggtaaggttatgcattctttcctccatgtgccagattgcccctaccccttactaggacgggacctattgaccaaattaaaagctcaaatacactttgagaggtcagaagtcaaagtcacagggccagagggaattccccttaccatcttgacaatgtccatagaagatgaatatagactccatgaaaagaggactaattcgaacaatcaggaaacccttgatcactggcttgcggaatttccccaagcctgggctgaaacaggaggaatgggccttgccattaaccaggccccaattatagtaaccttaaaagctgccatccttcctgcatccgtcagacagtatccaatgcctaaagaagcccgcgaaggaattcggccacatattaaaaggttacttgaacaagggattctggtgccctgtaaatctccttggaatacacccttgctacccgttaggaagccgggaactaatgactaccggccagtgcaggacctgagagaagtcaataaaaggatagaggacatacaccctactgtccccaacccttacaatttgctgagtggattgccacctaactatacctggtacacagtcttagatcttaaagacgctttcttctgcctccgcctgcatcccaccagccagcctatatttgcctttgaatggcaggacgcggcccttggaatctctgggcagctgacttggactaggctacctcaagggtttaagaacagccctaccctttttgatgaagctttacatcaggacctggcagaattccgggttaggtaccccgctctaatcctcttacaatatgtagatgacattctcctggcagccaaaaccaaaggggaatgcaaggaaggcactcaagccctcctccagactcttgggagcctagggtaccgggcatccgccaagaaggcccagatatgtcagaaacaggtgacctatttaggatacaagataaaggatggacgtcgatggctaacggaagcccgtatgcgagccatcttagacattcccaccccacaaaatccccgccaactgagagaattcttgggaacggcaggcttctgccgcctatggatccctgggtttgccgaaatggcggctcccctctaccccctcactcggccaggggttgcttttaaatgggaagagccccaaaagaaagccttcaccgacatcaaaaaggctctccttgaatcaccagccctgggtctaccggacttagctaagccatttgaactttttatagatgagaaggagggctatgctaagggagtcctcacccaaaaactggggccttggagaaggcccactgcatacctctccaagaaattggatcctgtggcatcgggatggccaccctgccttcgaatgattgctgctatagccctgctggtaaaagattctcacaagctaaccttggggcagcctttgaccatacatgcccctcatgcagtagaggcagtcatcagacagcctccagacagatggcttactaatgcccgaatgactcattaccagactatgctgttagacaaagaccgggtccacttcgggcctttggtgactctgaacccagccaccctgctccccctccctggggagcccgaggctcatgattgcttacaggtattggccgaggcccatggagcgagatccgacctgactgaccagcctctacctagcccggaccacatctggttcacggatggaagcagctttttgcatcaaggagaacgaaaggcgggcgcggcagtcaccacagagaatcaggtcgtctgggcccaggcactcccccctggaacttccgcacagagggcagaactcatagcactcacgcaggctctaaaattggcagaaggtaagaggctcaccgtgtatacagacagtcgttatgccttcgccactgcccatatacatggagaaatttacagacggagggggctgcttacctccgaagggaaagacattaaaaataaggaggaaatcctcgctctcttaagggctcttcatctgcccgctgccttaagtatcatacattgccctggacaccaaaaaggggattctttcgaagcaaggggcaatcgaagggcagacttggctgcccgagaggcggccctgaccacagacaccactaacctcctggctctagagcccaccaacgaccatcccttcccctcatgggactatgaacaaagagacatccaaaccctagagaaattgggagccgcaaaggaaccaaacggggattggacttatgaaggaaagactgtcatcccctaccgggtaaccaagtacctagtgacatttttacataagatgacacatctgagctccaagaagatgcgggagctcctcgaacgagaagaggaattcaatttccttttggggaagaacgatattctaaaacaggtaactgagcaatgtgatgcgtgcgcccgagtcaacgcatccagactgaagcttcctcccgggaaccgggtcagaggctaccggcccggaacacattgggagatagatttcactgagattaaaccaggaaaatatggatacaagtatctattaatttttgtagacaccttttcaggatgggttgaagccttccctactaaacatgaaacagccaagatcgttactaagaaattgcttgaagaaatctttccccgttatgggatgcctcaggtattgggaacagacaatgggcccgccttcgtctcccaggtaagtcagtcagtggccaccttgttggggattgattggaaattacattgtgcttatagaccccaaagttcaggacaggtagaaaggatgaatagaacaatcaaggagactttaacaaaattgtcgcttgcaactggcactagagactgggtcctcctactccccctagcactctaccgcgctcgtaatacccctggaccacatgggctcacaccctttgagatcctgtatggagtacctactcctatcattaactttcttgatcaagatgtctcagattttgctaactccccttctctccaagctcatttacaggccctccaactagtacaacgggaggtctggaaaccccttgctcaagcttataaagaccagagggaccatcccaccatcccccattcctaccagatcggggacactgtttgggtccggcgtcaccaggccaagaaccttgaaccccgctggaagggaccctacatcgttttgcttaccactcccaccgcactcaaggtagacggcattgcagcttggatacatgcttcacatgtaaagccagcccgacccaccgattcagccactgcatcagaatggaccgcacaccgcactcaaaatcctttaaagataagactctctcgtacaccctcctgttgattggttgtctgtttacccccatgtagcaactaacccccacagggtttataatatcacctggaaaatagccaatctagggaccggggaaatagccaacctcagcacttatatagggactctacatgatgggttccctcctctctatgtcgacctatgtgacttagtagggtctgattgggatccctctgaccaggaaccattcccagggtacggatgccaccaccctggggaaggataggaacaagaagcaaggatttttatgtttgccccggccataaaccaactcatggctgcgggggccgcaggaagggtactgtgcaagatggggatgtgaaaccacaggggaggcttactggaaaccctcttcctcttgggatttcatcactctcaaacggagggagatcccagggtacgcagggaaaggaccatggagatgtgggcaaagagcctgcggaccttgttatgatagtgccggaggggaggttttcaaggcgccacccccggaggaaaatgcaaccctctcatcctaaggttcacagatgctggaaaaagaactacttgggatagtcctaaggtctggggactcaggctgcaccgagcagggaaagatccggtgactttattctccctgtacagacaaattactcccctaagccaacaatcagtcgggccaaacatagtaatagcggaccagagatccccaacccattttcaagtccctaaaccccctaccgttcctaaagctatcactcctacaccaggtgctgtcaccttctccccaccccagatgccctaaacatcgagataaccagagaccctccaggtaccagagatagattattacaattaatccaaggagtttaccaagccttaaatttttcagaccccaacaagactcaggaatgctggttatgcctagtttcccggcccccatattatgaaggcgtggcaatactgggcaactactccaaccagacctcagcacctaccagttgcggagctgctatgcagcacaagctcacaatatctgaggtctcaggaaaggggctatgcataggcaggattccttcctcacatcaagaattatgtaaccaagtagagccattatctcaggacagccgataccttgttgccccttatggaacttattgggcttgcagtactgggttgactccctgtgtctctaccactgttctcaacaccaccattgacttttgtatattgatagaactttggcccaaagtcacataccaccaacctgaatatgtttacagcgtactagagaaatcaacccgatataagagggagccaatatcctttaccgtggccctattattaggaggaataacagtgggggcatagcagccggcatagggaccggaaccgttgccctacagggaattaatcattttaagcttctacaacaagccatgcacacggatatccaggtcctagaagagtcagtcagtgcactcgagaaatccttaacatcactctctgaggtggtcctgcaaaacagacgaggattagatttattatttttacaggaagggggctatgtgctgccctcaaggaagaatgctgcttttatgcagatcatacaggaatagttagggatagcatggccaaacttagggagaggctaaaacagaggcaacagctatttgagtctcaacaaggatggttcgaggatggttcgctaatccccctggttgactacccttatatccacgctcatgggacctctggttattctatttttgatcctcatatttggtccctgcattctgaacaaactgactcaattcatcagagaacgactatctgttgtacaggccttagtcttaactcaacaatatcatcagctaaagcaaatagatccagagtatctagagacctctgaatgaaagattccattcagttacaagagaaatggggaatgaaagacccctgtcccttagccctttctttctcaagtttgtctcctcttcctcctgtcggcggcttccccgatccccacccccggtggcctttccccgcccggcccgagaacaagcaccgggtggggccggcccgagaatgagcaccaggtgggccagcccgagaacgagcaccgggtgggctggcacgagggcgagcaccaggtgggtcagcacgagaacaaacaccgagtgagccggcctggagctctgcccctgagcccccgccccgcccgaagagaaacactccgtcccaaggtctccgcccccaaggtcagccatcaggaaagggggggaattgagtctgctgtaccagacaccagaccttgagaatatgctgatctggaatggctctgtgtctcatttgaaccatccaatggaaatgattctgtatttcgcctcatttgaaagactctgtgtttcacctcatttgaataactctgtactttgcctcatttgaataaccctgtatagcgcctcatatacattgaccaatgggaatagctctgtataatgcctcattagaattatccaatagaatccttgctcctagcttgcgcctttttcctatataaggacccctttcccttggctcgggcgcttagccacacagaagctaagtcgccccggtaccgcgtctccaataaagcctcttgttttttgcatccagttcgtggcctcgctgattcctgggtgtgtgggtctccctctacgaaagtatcccttcggggtcttgcatggtgtgatttgaatgagaaatgcccctataggctcagatgtctgaatacttggtcccagttggcagctgtttggggaggttgtgaaatctttggaagtgaagcctcattggaggagtagtcactgggtgggctttgagtatttatatgctcccccacttcctgctccccctttctctttctctctttctgcttcctgttgtcaagcttcccaaacatcatggactcttacccctctagaaccataagcccaaataaacagtcttctgcttattggtcatggtgttttctgttgttggtcatggtgttttttcacagcaacaggaaggtagctgatacaacagtctctgtgactgccctctgaccaccccacgcagaggcttcagaactccttccactacactgaaaagtcttccaagtctccccaagccatcattatgtgaccataacctgccaattggatgcacttaagactgttgccagggtacaattctgggtccttttctaacattgttttccttgctccctgaaagtttctattcttctgccctacttccctagcttcctttagactcaccaaacagttctatgtttctgtggggcaaattcctctggggcacagggggtggggtggggagtgtccgtaactttagcctgattttatcttcagactttttttttttttgctggctcaaaaatcatttcctagcctaccttgaagcttggagggcctggtgattaagttcctaacagagggatgcatacagaagaatcagttgagactttcaagagaatgtaccacttagtctcctctttgctaaatgtgtgtgtgacagccagaactttaccactacaagctgatcttgagaatggaggtcatgtacagcagagaagatacaagggggacatttggaactctgttttcttctttctagtttggactttgcttacatgacagagcagagccccgtgtgtttaccatcttgtaataaataaatatatagaaatgctctgcagtaaagctcctttgacttaatacctgcaggttgggttatgtagccaggtctcactatctccacttctactgctctctcaccacagtcttatgccacatgtatagggacacatgttttgtctgcttttttcatgatcccatcaagtctgcatggcatccaatgcttctgcctctgctactccctcagcctgggatgtccttcctaaccttgtctctttgggtggctccatgtcaaaattacctgtttacttgtctgttctccaccagacaggtacaacctgatatttagcatctgctccaggtgtgtgctccgggctgagtgcatggggtttgaatgaggaaacagagtggatgttgcagactcaggaggtgggactgactttacactctgaaaccagatccctcagtttcttcccaccacctgctgccccctggctttatgcagacgcagggagggggccatgtgttagcagagcatcttcttgccacaaggcagtgccatcccttatacatgccagcacaggcctgattttgaatggaccgtgtcagacttgaaggtgttcagaatgttaacttgactacctcaaaaaacaagtacaatagtaactgattattacttccctctctacaagtctgtacagatacaaaaggctggataaaatgaacacattggggagaagcatgaagttggccatatgtggtgcatttctatcactctgcatgctccaagcttaggtgtggccattccagtgacttcctcccaaagcacaaggtttggacatcagggagaatgacagacataagcaacctccaggcagccacagttgataacaacaggcacatgtcatgttaacagcgtgtccttagtatgctgtgacagtgatagtttccttggggtcttcttcccaatggccaataacacctgtcttttcataggcagaacgtcagacaaatgctattagtgagtggccatagaaaatgcccaacaggaccccctcaacactccacaaaaaagcaagacacactgggcatgttcttataaaaaaaatgtagccttgcctttaatcccagcactcaggaggcagaggcagatggatctttgtgagtttgaggctagtctggtctacaaaatgaattccaggatagttagggttctgttacatagagaaaccctgtctcaggaaaacaaaacaaaacaaaacaaaaaacaaaacaaaacaaaacaaaaaaaacaaggccaaagatgccactaggtgggctctggagggactatttattgatgggctcttgccaagctgggtgctgttcatggagataccccagcccttctgcctcttgtctgtgaaatccagacacatgtccgtaagcatcagctatgaccctgtctcaaagaaaactccagtcttatttatcagctccacgctctaaccaactgagctagccggccagactccattcttatttatatttaatataagattttgatactcataaattatagttttacttagcttttttgttaaatagaagtttataagatatacagattcttatttcataaacagaggaaaatgtattttctctttttgagacagggtctcactgtgtagaccagactagtcttggaatcacaaacctccacttgcctctgccctctgagtgctgggattaaaggtgtgtgtcaccacacctggccagaagatgttcttattcacattgcctttagtatatccttctttggtggaaacctgttctcatgtgtacatattgaacagactcacaccttaatttttttttagacagggtttctctgtattgttttggagcctgtcctggaactcgctctgtagactagactggcttggaactcacagagatccacctgcttctgcgccaccaacgcccggcacaccttaatttttatttttgtgtatgttcatgtacaagcaaatacatatggaggccaggggtcaatctctggtgtcattcacgaagcactgacaccttgtgttttgaaacagggtatctcatgttctggatcttgtcaagtaaactagactggctggccagccagccccaggaatgcctctgtgtcttcttcctcagtgctgcaattaaaagtaggagccaccttgcccaacttttactgggataacaaggggtcaaacacaggtcatcctgcttctatgacagacactttaccaactgaacgatctttccagtcctaccttttgttttttaatctttgtaacatggtgctgctcatggtgagatacatggagcacagtggggcctgaatgtggtgccagtccacagtgaatcaaggatggaaacaaagctctttacaaactgccgaagcaacttcacagaactttcagctccagttcctgggcttgcagccttcctttctcctttcacagatgagaaaatgagcaggaagggtgaccagcctgtccattgggttgtcacgtgaggaaggtgggtctgctgggtcacactcttcctgcttcttgctctcttttccctagt

SEQ ID NO: 17 - DNA, 11089 bp, Cricetulus griseus
  misc_feature: 000312F-023-0115000-35000_Type_C
gtggtccctcaatttggctgcctacacaaaatctgaacaaggacaacacattaataggcatgtgggcatggaagagggaaatttcatgggtccccatccctagataaagactaaagaaacataaggaattctgggtgtggggattagtcttccttggggatgtgccccttaattggttatcccataccaagtggtcaaccctaaaatcatattcatacaatcaacactaaatggattcaccaggtcataattatatatttattcatactatatttatgtaacagtaacaaggaaaatgcagcaagaggggtgcatgaaagggctggagaaaggaaaaagtgatacattttaatttagaaaaactaaggagatgatttatagaggacataaatagccaataaatacattaaagtgtccaacttcttttagtccttaggaaaacacagctccaaactatattgatattccatctcacaatgtcagaaatgtcatcaagaaaatgaaatacaaggaaatgttggtgaggattcattgagagggaatatttacataatattggcaggtatgtagtcagtgcataaaaatcagtatggtgattctgccccaaacaaaaccacaattgcaatatgatccatccatatcacactcagtacagattcaagagaatataagtcaggatatatcacagatagctgtgtaatcatgtttattatagtatcagttacaatggctaaatacatgaatcagcctaagtgccagcccatggatgaataagtaaagaaaatttgatgagtatattcagtgtcatattattcagccacaaagaattaaattatgtcattttcagaaaaatggatggaattgtagatcatattaggtgacataactagacttagataagtgttatgtattttcattatatattaactgtagaaaaatgtgaaagtaaaaagattgaaaggaggtatgagaatgtagtgggtaaatatgtataaattcaatatatgtatgaatgaatatgttacagtatatgttaattgaaaaaaaggaaatcctcttagaagtactagttcatggggaccagagaaatggctcactgggtaagaacacttcctgctctatctgaggacccaagttctgtgacccagcatccatataaggaggctcaaaacatctgtaactctagcttcagagtatccaatgccctttttcctgacttctatgcctctctctctctctctctctctctctctctctctctctctctctctctctctcacacacacacacacacacacacacacacacactcatggtggcatatattcacacaagcacacatgtgtctatataaatacaaatctttaaaagagaatcagtagttcataagggaattataatttgcatcatacagataaaaggagaagaatataagactactctatacagacaagtaagtatagcttgagggaagagatatatatggcttgtatgaaataatgaaacctaatacttcctattgttttcttaaacaagagtagacaaagtatttgttgttggggcccatggaagttgttctgttttgtcttaaagtcagagttcagacctatcgttgttctttaaaggttaaaggcctaagacctacaggtctaaggtgtagttactgaaggatgtttggcttaaagtgtggatttctcaatcagataacagaatacttgatctaaggttaactagatgtcttaatcctcgaggctggataacaagaggtttgatctaaggtgtggctactaaatgttgtgagaattaaggaagtacttatgctgtttgttccttgtatcatggtaaagaagtcttttgcctttccccctctggttggggatataagaactttgagcaataaacttaaggcagtcccagtattcactggatgccttccgactctatcttctttctctagcctgtttctttcctcagtctacactccccgtctgtaggtatgaaccagacaagtcagctggatatgacaatttatctaatatcaaataatgctacttggttttcacaaagaaaacaaatagataaataagtcccaactctggatagccactttatgcccaaggaaactaagtcctattaaaaagatcatatcttcacactctcaggcctagtttcagccatgcaacctacataacaaccaagccaagttccccaaactcctacctgaatcctcaccacactcttgggccttgctcttatcatggaggttgaacccagagacacaactatgagtcatatttgaagtccttcttaactgccactgaattagaagcttagcattgtgtcttggtccctatgtctaacacaggtgatttgggaggctctcaaagactgagatactgggaatattatgggagaatagcagtacttttatgtaagtttaaggaacttaggagcaagaagcaatatatatgatgtagtacaggggctaaaatagcagtgaatgagatgaagaagccagagacctttgattccttcaacagatctggaaagaaaaaatgattggctgtagctattatagttgaactcaatattcggtagacagggcaaatatggctattagtgaagggccatgaagtaagtaatttctcaaacatttatccagaaagtccccagtaagtggatggcacgtgactaagtgattaaaaaagaaaaccaaaaacacaggacaaacaaacaaaaacacagcctttaaatggtcctaacattttctttctaaaaacatgattaacatttgggagcatgtgttatacaggaaattctttctaaatgaaaatattaaatgtgagcacttgttaacaaaatgcacattgaagaaaagaaaaaaaatacacactgaacatctatcatatgtatatataattgatactcttttaagtttctaagtaaataaaatagtcctttttacgtggacatgaaagacctcagaagaggtactttcgtagaaggagacccgcacccaggaatcagcgagaccacggacttggatgcaaaaccacaagagttttattagagacacgggtacccggggagacttagcttctgtgtggctaagcgcccgagccaagggagagaggtccttatatagcaaaaagcacagtgcagaagcgaaaagcatagttacagggattctattggacaattaatgaggtacagagcattttctcaaggtttgataattaggcgaggcgccttggctctatctcatctgggtacaacagactcggcagttcctcctccgcgaccagatggctgaccttggatggagcgttccatcggacggagcgcggtgccaggcggcaggccggggcgcgagcccaccgggtgaagcttggagggagctggtgccgagcgtgcggcggcggggaagtggaggccggcgggtgggatcgcgcgtgcccgccagccgccgacgaggaggaggcaaacttaagagggctcagggggggtctttcattcccccatttctcttgtaactgaatggaatctttcattcagaggtctctggatattcggatctatttgtttagctggtggtattgttgagttaagactaaggtctgtacggcagatagtcattctctgatgaattgagtcatcttgttcagaatgcagggaccaagcataaggattaaaaatagaataatcagaggtcccatgagagtggatataaggtagtgaaccaggggacttagcgaaccatccttcgaaccatcctgttgagactcaaatagctgttgcctctgttaagtctttctctaagtttggccatgctgtctctaactattcctgtgtggtctgcataaaagcagcattcttccttgagggcagcacatagcccccttcctgtaaaataataaatctagcctcgtctgttctgcaggactacctcagagagtgatgttaaggatttctcgagtgcactgactgactcttctaggacctggatatctgtgtatatggcttgttgtagaagcttaaaatgattaattcctgtaaggcaactgtaccggtcctatgccgctgctatgcccccactgttatcctcctaataatagggccacgtaaaggatattggctccctcttatatcgggttgatttcctagtacgctgtaaacatactcaggttggtggtatgtgactttgggccaaagttctatcaatatacaaaagtcaatggtggtattgagacagtggtagagacacagggagtcaaaccagtactgcaagcccaataagttccataaggggcaacaaggtatcggctgtcctgagataatggctctacttggttacataattcttgatgtgagggaggaacctgcctatgcatagcccctttcctgagacctcagatattgtgagcttgtgctgcatggcagctccacaactggtaggtgctgaggtctggttagagtagttgccctgtactgccacgccttcataatatgggggccgggaaactaggcataaccagcattcctgagtcttgttggggtcgaaaaatttaaggcttggtaaactccttggattaattgtaataatctatctctggtacctggagggtcctggttacttgatgtttagggcgtctggggaggggagaagataacagcacctggggtaggagtgacagctttaggacggtaggggttagggacttggaattgggttggggcctctggtccgctattactgggtttggccgactgatgttgcttagggagtaattgtctgtacagggagaataaggtaaccggatctttccctgctcggtacagccggagtccccaactttaggactatcccaagtagtcctttttccagcatctgtgaaccttaggatgaggggttgcatttcctccggggtggcgccttaatatttcccctttggtactgtcataacagggtccacagattttaagcccacatttccgtgaccctgtctctatgtacccgggatttccctcctttgagggtaatgaagtcccaagaggaggagggtttccaataagcatctcctgtggtctcacatccccagcttgcacaatatccctcctgtggcccccgcatttgtcggttgtttatgaccggggcagacataaaaatcctttctcgtgttctcatccttccccagggtgatggcatccgtacccgggaatggttcctggtccgagggatccaatcagaccccactaagtcacataggtcgacatagagaggagggaaccatcgtgtagagtccctatataagtgctgaggttggctacttccccggtccctagttggctactttccaggtgatgttataaaccctgtggggttggttgctgcatgggggtaaacagacaaacaatcaaaggagggtgtacgagagagtcttatctttaaaggattttgagtgcggtgcggtccattctatgcagtggctgaatcgtgggtcggtggctacatgagaggcattccagctgcaatgcttacttgagtgcggtgggacagtagggtgtatgtcctctatccttttattgacctccctcagatcctgtactggcctatagtcattggttcctggcttcctgacaggcaacaaggtgtattccaaggagatttacagggcatcagaatcccttgttcaagtaaccttttaatgtgtggccgattccttcgtggctctttaggcatcggtactgctgacggatgcaggaaggcggcagcttttaaggttactataattggggcctggttgatggcaaggcccatccgcctgtctcagcccaggcttggggaaattccaagccagtgtcaagggtttcctgttgctggttagtctcttttcatggagtctatattcatcttctatggacattgtcaagatggtaaggggaattcctctggccctgtgactttgactttgattctcaaagtgtatttgagctttagtttggacaataggtcccgtcctagtaagggtagggcaatcaggcacatggaggaaagagtgcatgaccttaccggtcgcgagctggagctgccgatctgtggtccaacggtactgctttccgcctgtagcccctgtacccatgcagtccggtgactcaggggtccgggtgactgattcagaacggaatgttgtgcccctgtatctactaggaaggtgaccggatgccccccgacttgcagtgttatcctgggctcaggggggggggctcctggccctgacttctctaatcttctaggtttaggaggtcagaggttcttggccgtggaggcttggcccggggttcttagggcattcttttgcccaatgtcctttttctttgcagtaagcgcattgatccctgtccaggtggggcccccttctgccatccctctgcctattctgtctctgacctgtcactactgtggccaatagcctgctcatctctctattccgcttacggtctctcgcagcctctttctcctctgcttcttttctcagtctttcctctctttcctgggcttccttcctccaacgttcttctctttctgtctgtgtctctctcttattaaaaatacgttctgcttccttaacaaatcttggagcgtatatccctgtaattttctagcctttgagcttgtttcttatgtccgggctgactgccaaataaaggacatagaaacgctggtggcctgcctggatcttccggatcatagggagtatacatctatatgcttcttttagtctctctagaaaagtggcgggcgattcctccgcccctgtatcacctgctttacctgggccaaattggtgggtcggcgtcctgcccccggagacccgctacaagcaactggcgatagagacgcagatgggtcctacctgcttcagtgtaaaatcccattcaggtctttcaagagggcaagccgcatcgatttcattgggcagctgggtgggctgcccgttggctcctgggacattctttcgtgcttctagtagaacgcgctgtttctcctccgaggttaagaggacctgcaaaatctgctgacaatcatcccaggtaggttggtgagttgtgaggaccgactctatgagagatgtcagcctcacagggtctgcagagaaagaaggattgttattttccagttatagaggtccgatgctgagaatggccaatattgaggttggccattcggtccagttcggaggggcaaagtggttgaatctggaacgggctgctccctgcgacctcttagtcggtgagccattggggaagggtcaggtggggagcggggccgcctcgccgaggcggcgggggccgcttctgcctctggcgggggcggcggtgcggatatggcggggttcctcagtcagtaggtcaccaacagtttctcgggggagacctagggtcttctttcttcagctttagcgtctttacctgtagggtaaaggctggagtgggaggagctggggattggaaagggtaggggcgagggagtgggcggtggagcgttagagggggaaggaaggggggcccttggggtgtaggaaaggacgtaccaggggggggtcctgaaccaaggcctcccaagtgactatgtatgccacctggtcgggatgcccctgtggtccaggatccatagtcttctctttcacctgaaatatagtttggggttaaaggtaccatctcgcggccagcccacgtcaaaggttggccattctgaggaacaaagagtaatccatttgcgtttacgcacatcaacagactggttatgagcataatcctgcacatctctccagtgcgagagtgttagggacaaaggagtggtgacagtttgcccatagtttctagactaggagacacaaatgatggttacaagacaaagactaacaaacagaacaggctttcgcgcgcggtttcgacaaggtcgaaagtaaaattcagatggggcgaagaagggggcctccttctcgtccccgatgaggatagcaaatccctcaacctgagggcttcccaaacagattgggaagatgcctagcatctacctagtcagaaaacagaagtgagcccactgaggctcaacgctaccaggacgtctcctggagccggtcggagtccgaaccgtcagtcctcgagcccaatacaatgtacgcagctgtgtactacaacgcaagcgcagttcataaacggattcagacagacagacagacaagacaagacaagacagcggatccgcacaacacttacctcccaacgtgggtcggtggtccctgggcgggtctcgatcccggacgagcccccaaatgaaagactcagaagaggtactttcgtagaaggagacccgcacccaggaatcagcgagaccacgacttggatgaaacgcaagaggtttattagagaccgggtacccgggggacttagcttctgtgtggcaagcgcccgagccaagggagagaggtccttatatagcaaaaagcacagtgcagaagcgaaaagcatagttacagggattctattggacaatttaatgaggtacagagcattttctcaaggttgataattaggcgaagcgccttggctctatctcatcttgggtacaacagactcggcagttcctccctccgcgtccagatggctgaccttgggatggagcgttccatcggcgggagcgcggtgccaggcggggcaggccggggcgcgagccccaccggggtgaaggcttgggagggagctggtggccgagcgtgcggcggcggggaagtggaggccggcggaggtggggatcgccgcgcgccccgccagccgccgacggaggaggaggcaaacttaagagagggctcaggggcggggcctttcagacatatggacatatctcctacgaggaaggaggaggaggaaggtggcagaggctatccatcaatgccaggtatcttctccaagcatctcgaccctatcttaagatagggtctctcaatttagattgactgcactggctgaccagtgagtcccgatgatctactctggcagccttctccccagcactgggcataaactagcccagaaaatgcctggcattttctaaaatttcattctttcatttaaattagaaacaagcttgttttacatgtcaatcccagttccctctccttccctccatcctcccacccgcaccaaccctctattcctcccctttctgctccttagggagggtgaggccttccatggggatctttaaagtctgtcacctcatttggagcagggcctagaccctcccctatgtgtctaggctgagagagtatccctctatgtggaatgagctcccagagtttatttgtgtagtagggataaatactgatccactactagaggacccacagattgcccaggtctcctaactgacacccaggttcaggggtctggatcagtcctatgctggtttcccagctatcagtctggggtctttatgttgatggtggggatcaaacttagctctccatgcatacccaaagcacaattcttacagagccacttcctcagtcctgctttagtagtaaatgtaacaaacaaaattttctgaatctaagaatcatatcctttgtcttcaaaataaggagtgtttgcaaaggtaaaattacagactggaaattctgaaaagactgctgtagtactaagccaattttttaaaaaattcctccttcgtcttaaaagaaaaagaaatctaaattcttatgactctatagaggagtgtagcccagttcagttcaaggttgcccgcttctctgttactagctgttttctctaggtcctgctttcaagattaataggaaaaagagccaaatgggaaaatctttctctatccaaacagctggattagaggaatagcatccaataagatgactgagagttttattgatctagtttttcatattaaaatttaggtgagtcagtcagaagactacagtgtgtaaggaattatgttaactaacaactacaatttcaaagaaaatgtgaaaaattctgagtgatcagcagaattggtcagttctaccacaaaagaccctcatagtccatggattttgtttcttcatacttatctctctttagccctatggtcaaagctcatggcggggggtgggttgtggtcattcaaggacaaaacagtgaaaactttgagtcactggaatcacactgccaacaaacaaggtgacatgctgcctcttgtttccgctctcctaatacaaaccaatggcatttctggggtctttgtagtggtgttacctttacatttttgcagccgccaaaggccaatatggtaaaggtgggctccaccttcaagggtagaaccagtaagtcaatgggatattgcctcctcctttctccctttatttcctggctactatgaaatgaggaatattctgtagtagccactctcacaaggcagtgttgccttgccacatgccctacaatgatagggcagccattgactgaaatctccacaattgtgagacaaaataatgcctttttcttgaagctgatgttagacaatttgtttctgcaaatgcaaaagtaagtgcaaagagctttggtgacacttccaggaaaaaaaaaaaaaggcatatataagataagagttatagtgctaaagtcatgaaattagtgctgctgggtcagcaacatatttataaatatcgcaattgtaaataaaagcatatgttatacattgcttggctgatgaaaatgtgatcatcagaagcttacagcaacctaagcttataattctcctagaatcatcagtttggtactcactattagacagtgcccagacatatacacacatacatacatctaaataagaatttaaatgttagagctaacctataatgggagcaataatatccttcctagacaccacaggataccaaataagaaaccaagtgtcaggaatggattacctctttgggttttattgacaagtgaaatccatagtccttgatatatttcaggctattgccaatgctcctggttgccctccagaacttgacactaagaccatctt

SEQ ID NO: 18 - DNA, 17295 bp, Cricetulus griseus
  misc_feature: 000322F-036-0115000-35000_Type_C
taaagcaattttacatggataagctagtaagcttagagtcttaactattgccatacttaaaaaaaaaaaaaaaagagtttttgctatcctatcccaacaacttgatgagtctcagttctgtaggggatgaagagcatacacctggttcgttctttaggggaaatccctcatgccaggtgattgttgggcctgaagagttttctggagactcaacagggctagcgcacccaaaatggcctctgaagtcacatggccttatagctcttccacacagggtccatctccaggcagcctcctgactcctggatattgcatgtggctcctttgtctcgtagagtagtaatctggccattttgcagtccagttggtttgcagcagtacaaagactaacctaacctcccaggatgtcttagtctggatagactgtccatcattcctggtgccttctattgagtaacttcagctttagaagcccctagatccactgtgggatcaacccaaaagaaaagcctaggtgatgagattcattaatttctgctatccttttagactagcagaaattgaagaatttcagctactaatttcagcaaatgggaacttcatgggcctgatggatcagaatgtttcctgttgccttcatgcagaactgaattccacaaagctcagtgaccaaatctgaatgatgtaaactcaacaaacattgccctaaatcaggcatgtgactttgtcactttagaattaaacagcagctcttttctatggtgtttcctagactgtgggctgtcaagatgaagctgggtatctccttcccagccaccagctatcagaaactcattaaagtggatgatgaacccaagtttcatacttcttatgagaagcacatggccacagcagtggctgctgatgttctaggtgaataatagaagggctatgtggtctgaatcagtggtgggaaggacaaacaaggttttctcatgaagcaaggtatcttgatccacagcagagaaaccctcatattgaatgtggaacattcttgttataaaccaagaagtagagaagagaaagtacaaatctgttcatgtttgcattatggataccaataccttgatttgggttctataaaataagtaagcaatcaatcaattgatcaataaatacaagaatattcctagactggggcctaaaagagctagcagaatctgaaagcttttcaatctctctaaagaagatgatgtcccctgatatgttgtcacgtggtccttaaataaagaagatgagatgctcaggactaaagcccccaatattcagtatcttgttactccacatgtcctgcaacaccaatgccaatgtactgctctaaaaaaacaacatactaagaaaaacaaggaggaggctgcagaatgtgctaaacttttggccaagagagtaaaagaagccaaagaaacatgccagggacagatggctgagaagcacaggctgtctgctctgaggatttctaagtctgaatcaaaataattccttaagaattaatcaaaaacaattctttatgagtaacaaataaataatcagatctttaatctcagaaaaaataatgaatgaaataatacaatatttttgctaaaaactgtctttgtatcttgaggtcatcccatgagagagagtccatgccctacccatcgcttggaccatcagcaacctgggactggacagcccagagtcttagggtagaaccaaacatgatggtcagaaagaaagagagaatgaaaaaaagaaagaaggaaagaaagaaaggattcctgacaatattctgctatactcatagattagtgacttgtccagtctccatcagagaggcttcctctgacagggggtgggagtggatgcagagacccgcagccagacattatgtggagagagagtctaaattagagatctccatccaccccagagaagcccccagaagaggcagatgaaagaaagattgtaggagtcaaaagtaacggacaacactaggagaacatggcccattaaatcaactgagcagggaacgggtgggttcacagagatggaaggggcaagtgtgggcctgcatgggtctttccctcctattgggttgcctggcagtccagccttggtatggggcttttgccttgtcttattggatcttgtttcatcatgtctggttgttgtctcttggaggcctactcttttctgaagggagactgacagggaatggatctgggggaggagtgtggcagagctggcagacgtgtattgatgagaatacacggcagtcagggtgtattgtatgagagaagaatctattttcaactaacaaattagccttgtcaactaagaaagtttatctttctaaaacacaaatacagtattttaggaacctattgaacatacaattttcaaaagattaaaaatcatcaataaatatttttttctcaagggatttcatatttttttcagatttcaaatcatttgagaatggaaagatgtgaagaatagtaatgtatgcaaaaataaccaatatttctcattttctgtatgataagattccctttgatcatggacatgagctaagaatatcttaagatataatagaacagtcactcctacaggaaggcaagatggctgggtgggtaaaggcacttgccaccaatcctgaaaatgtgagtgctgtgaaataatcccattgtacactgtaaagatctgtctctcaaattggtataataaaacactgattggccagtagccaggcagaaagtataggcacagtgatcaaagtaaggatggtgggaaggagaagggcagagtcaggggagacacgggccagctactgagcaagcaggatgggtagaaaatgaggtaacaagccatgagccacatggcaaaatatagatagaaatatgggttaatttaagtgtaagagttagctagtaacaagcctgagctatcagacaagcatttaaaattcgtattcagcctctgagttggttatttgggactggttggtggggacaggaaatttccatttacatgtgagtttgatttccaggattcttatggtagcaggagagaactggttgctgcaagtagtcctctgacctccatactcatgtaataatatgcaaataactgtaatagaatagttttttttaaggaaaaatagattatatttgattggctctcccactcagttgaagggatttgacttttcctgggcactgaggttttatcagtgaaagtcaccagataaaaggaagattctctgtcactgtctttaaagcaaagcggaaaacatgacagaggactgagtgttaaaatgtcatttgtagccctgactgacagccagcaacccatcgtgaacctcagatttttaaaaattaatttattttttgaagcagggtctcatgaaaaccctgtctggccttgagatcattatgtagtcacggatgaccttgaacttctagctgtatggtctctacctactaagcaccgggacgacaacgttccctgtttatgaggtgctggggagcacctatagaagacctcagtcctataactaaaaggaaatgaactttgacacaagctgtgtgagttgggagagcacaataaacgtaagatgaccatgcaaccagatgatgtgtaggctccagccttgtgaaaacatgaggagagagctcagttgtacatcagctgtgggcacatgcagaagacacttagatgggaaggccacctgtgctgtgcttgaacatctgacctacagaatgccaatgagtttgctgtatttgtttctcagcagcagaaaatgtatctaataattgtttttatgcaactcattatactgctatatatctgaaacgaaagtaaattatagaaacgtttcaaggatgatatgaagaagaatattgcctattatttactgcatatgtccgtgactaaggttttggttttgaaatgtccacccccaagaaattgcccaagaatttttttttttttacaaaccaaactgtacccaactttatgaaagatatttttcataaacaatcatagcatttttttttgagacagggtttctgtgtagctttggcgcctatcctggaactctgtagaccaggctggcctctaactcagagatctacctgcctcttcctccaaatgctgggattaaaggtgtgcgccactacagctcagcaagcaatcatagcattttaggcaggattttggcatacaattgctaacagtatacaagaactttcaaactctggtcttcttcatttttttcaattatatttgtttattttatatgtgtgggtgttttgtttgcatgtatgtctgtgcaccacgtgcatgcctcggtgccctgggaggccaaaagagggcattggattccctggaaatgatttcgaacaccatgtgggtgctggaactgaatccaggtctcctcgaacagccagtgctcttgaccactgattcatctcttcagcccttgtatggactgccaaaatgagaaagccactctaacaccagtggaatcttcatgtgatgctctgaacagggaaaggctaggtcaagtagcctttctttcatgtcaagcatgctgtctaacaatgtttcacaagacagccccacttccagaagtgagatgaaaatggcacaatggtcgtctgcagatcctcatcttgtaggaaaggaacatttcctttttgtgaagaacaccacatcagtgatgtcactggcaagttcagattgcccaacatactggcaaaccaacttaggggccaggtccagtgaaagacccctgtcccttagccctttctttctcaagtttgtctcctcttcctcctgtcggcggcttccccgatccccacccccggtggcctttccccgcccggcccgagaatgagcaccaggtgggccagcccgagaacgagcaccggtgggctggcacgagggcgagcaccaggtgggtcagcacgaggacaaacaccgagtgagccggcctggagctctgcccctgagccccgcccgcccgaagagaaacactccgtcccaaggtctccgccccaaggtcagccatcaggaaaagggggggaattgagtctgctgtaccagacaccagaccttgagaatatgctgatctggaatggctctgtgtctcatttgaaccatccaatggaaatgattctgtatttcgcctcatttgaaagactctgtgtttcacctcatttgaataactctgtactttgcctcatttgaataaccctgtatagcgcctcatatacattgaccaatgggaatagctctgtataatgcctcattagaattatccaatagaatccttgctcctagcttgcgcctttttcctatataaggacccctttcccttggctcggcgcgcttagccacacagaagctaagtcgcccaggtacctgcgtctccaataaagcctcttgtttttgcatccagttcgtggcctcgctgattcctgggtgtgggtctcctctacgaaagtatcccttcggggtctttcatttgggggctcgtccgggattgagacccgcccagggaccaccgacccacgtctgggaggtaagtgttgtgcggatccgctgttttgtcttgtctggtctgagtctgtcttgtgaattgcgcttgcgtttgtagtatacagctgtgtacatttgtaggcggatccgaggaggactgacggtcgaactcccgaccgcggctccaggagacgtcctggtagcgtttgaagccctcaggagggatttgtatttgaacttggaagcctcaggagaggatttgtatttgaacttgggaagccctcagggtgagagatttgtactttgaacttagatctatgactggacattttcccagtctctttggagaaggccctcggcttgagggatttgcaatctttactggggacgaggaaggagggcccccttcctcgactctctctcaattcttctgtcgactctctgtcgaaaccgcgctgcgaaagtctgttctgtgttattcggtctttgtcttgtagctgtcatttgtgcctcctaagctagaaactatggggcaaactgtcaccactcctttgtccctaacactctccactggaagatgtacaggaatatgctcataaccaatctgttaatgtgcgtaaacgcaaatggattactctttgttcttcagaatggccgacctttgacgtaggctggccgcgagatggtacctttaacccccagactatattccagataaaagagaagattatggatcctggaccacacgggcatcccgatcaagtggcttatatcgtcacttgggaggctttggttcaggacccccctccctgggtacgtcctttcttacatcccaagggcccctctctccttccccctctaaccgctccaaccgacccattccttcggcccctacacctcccactcctttgattcctcccaacccccttcccattccaacctttaccctaccgtggtgaaagacactaaggctaaagaaaagaagacacctaaggtactccctccgggagaagaccagttggttgatctattaacggaggagcccccgccatatccgccactgccgcccccaccagaggcagaagcggactccgcgctgccttggcggaagcggcccctgacccttcaccaatggcttatcgactaagaggtcgtagggagcagcccgttccagattcaaccactctgcccctccgaactggctgaatggccaacctcagtattggccattctcagcatcggacctctataactggaaaaataataatccttctttttctgcagaccccgtgaggctgacatctctcatagagtcggtactcacgactcaccaacccacctgggatgattgtcagcagcttttgcaggtcctttaacctcggaagagaaacagcgcgtgctactagaagcatgaaaaatgtcccaggagtaaacgggcagcccacccagctacccaatgaaattgatgcggcttgccctcttgaaagacctgaatgggattttaccaccgaagcaggtaggacccacctgcgtctctatcgccagttgctgtagcgggactccgggggcaggacgcagacccactaatttggcccaggtgaagcagataatacagggtgcggaggaatcacctgccgcttttctagagagattgaaagaagcgtacaggatgtatactccctataatccggaagatccaggtcaggccaccaacgtttctatgtcctttatttggcaatcagccccggacataagaaacaagcttcaaaggctagaaaatctacagggatatacactccaagatttgttgaaggaagcagaacgtatttttaataagagggaaacacagacagaaagagaagaacgttggaggaaggaaactcaggagagagaggaaagactaagacaggaagctgaggaaaaagaggttgcgagagaccgtaagcggaataaagaaatgagcaggttattggccacagtagtgacaggccagagacagaatagacagagggatgacagaaggggccccacctggacagggaccaatgtgcttactgtaaagaaaaaggacattgggcaagagaatgccctaagaacccccgggccaagcttccacgccaagggtttctgacctcctgaacctagaagattagaggagtcgggccaggagcccccccctgagcccaggataacactgcaagtcgggggcatccggtcaccttcctagtcgatacaggggcacaacattccgttctgaatcggtcacccggacccctgagtcaccggactgcatgggtacagggagctacaggcggaaagcagtaccattggactacaaatcggcagctccagctcgcgaccggtaaggttatgcattctttcctccatgtgccagattgcccctaccccttactaggacgggacctattgaccaaattaaaagctcaaatacactttgagaggtcagaagtcaaagtcacagggccagagggaattccccttaccatcttgacaatgtccatagaagatgaatatagactccatgaaaagaggactaattcgaacaatcaggaaacccttgatcactggcttgcggaatttccccaagcctgggctgaaacaggaggaatgggccttgccattaaccaggccccaattatagtaaccttaaaagctgccatccttcctgcatccgtcagacagtatccaatgcctaaagaagcccgcgaaggaattcggccacatattaaaaggttacttgaacaagggattctggtgccctgtaaatctccttggaatacacccttgctacccgttaggaagccgggaactaatgactaccggccagtgcaggactgagagaagtcaataaaaggatagaggacatacaccctactgtccccaacccttacaatttgctgagtggattgccacctaactatacctggtacacagtcttagatcttaaagacgctttcttctgcctccgcctgcatcccaccagccagcctatatttgcctttgaatggcaggacgcggcccttggaatctctgggcagctgacttggactaggctacctcaagggtttaagaacagccctaccctttttgatgaagctttacatcaggacctggcagaattccgggttaggtaccccgctctaatcctcttacaatatgtagatgacattctcctggcagccaaaaccaaaggggaatgcaaggaaggcactcaagccctcctccagactcttgggagcctagggtaccgggcatccgccaagaaggcccagatatgtcagaaacaggtgacctatttaggatacaagataaaggatggacgtcgatggctaacggaagcccgtatgcgagccatcttagacattcccaccccacaaaatccccgccaactgagagaattcttgggaacggcaggcttctgccgcctatggatcccgggtttgccgaaatggcggctcccctctaccccctcactcggccaggggttgcttttaaatgggaagagccccaaaagaaagccttcaccgacatcaaaaaggctctccttgaatcaccagccctgggtctaccggacttagctaagccatttgaacttttatagatgagaaggagggctatgctaagggagtcctcacccaaaaactggggccttggagaaggcccactgcatacctctccaagaaattggatcctgtggcatcgggatggccaccctgccttcgaatgattgctgctatagccctgctggtaaaagattctcacaagctaaccttggggcagcctttgaccatacatgcccctcatgcagtagaggcagtcatcagacagcctccagacagatggcttactaatgcccgaatgactcattaccagactatgctgttagacaaagaccgggtccacttcgggcctttggtgactctgaacccagccaccctgctccccctccctggggagccgaggctcatgattgcttacaggtattggccgaggcccatgggcgagatccgacctgactgaccagcctctacctagcccggaccacatctggttcacggatggaagcagctttttgcatcaaggagaacgaaggcgggcgcggcagtcaccacagagaatcaggtcgtctgggcccaggcactccccctggaacttccgcacagagggcagaactcatagcactcacgcaggctctaaaattggcagaaggtaagaggctcaccgtgtatacagacagtcgttatgccttcgccactgcccatatacatggagaaatttacagacggaggggctgcttacctccgaagggaaagacattaaaaataaggaggaaatcctcgctctcttaagggctcttcatctgcccgctgccttaagtatcatacattgccctggacaccaaaaaggggattctttcgaagcaaggggcaatcgaagggcagacttggctgcccgagaggcggccctgaccacagacaccactaacctcctggctctagagcccaccaacgaccatccctcccctcatgggactatgaacaaagagacatccaaaccctagagaaattgggagccacaaaggaaccaaacggggattggacttatgaaggaaagactgtcatcccctaccgggtaaccaagtacctagtgacatttttacataagatgacacatctgagctccaagaagatgcgggagctcctcgaacgagaagaggaattcaatttccttttggggaagaagatattctaaaacaggtaactgagcaatgtgatgcgtgcgcccgagtcaacgcatccagactgaagcttcctcccgggaaccgggtcagaggctaccggcccggaacacattgggagatagatttcactgagattaaaccaggaaatatggatacaagtatctattaatttttgtagacaccttttcaggatgggttgaagccttccctactaaacatgaaacagccaagatcgttactaagaaattgcttgaagaaatctttccccgttatgggatgcctcaggtactgggaacagacaatgggcccgccttcgtctcccaggtaagtcagtcagtggccaccttgttggggattgattggaaattacattgtgcttatagaccccaaagttcaggacaggtagaaaggatgaatagaacaatcaaggagactttaacaaaattgtcgcttgcaactggcactagagactgggtcctcctactccccctagcactctaccgcgctcgtaatacccctggaccacatgggctcacaccctttgagatcctgtatggagtacctactcctatcattaactttcttgatcaagatgtctcagattttgctaactccccttctctccaagctcatttacaggccctccaactagtacaacgggaggtctggaaaccccttgctcaagcttataaagaccagagggaccatccaccatccccattcctaccagatcggggacactgtttgggtccggcgtcaccaggccaagaaccttgaaccccgctggaagggaccctacatcgttttgcttaccactcccaccgcactcaaggtagacggcattgcagcttggatacatgcttcacatgtaaagccagcccgacccaccgattcagccactgcatcagaatggaccgcacaccgcactcaaaatcctttaaagataagactctctcgtacaccctcctgttgattggttgtctgtttacccccatgtagcaactaacccccacagggtttataatatcacctggaaaatagccaatctaggaccggggaaatagccaacctcagcacttatatagggactctacatgatgggttccctcctctctatgtcgacctatgtgacttagtagggtctgattgggatccctctgaccaggaaccattcccagggtacggatgccaccaccctggggaaggataggaacaagaagcaaggatttttatgtttgccccggccataaaccaactcatggctgcgggggccgcaggaaggtactgtgcaagatggggatgtgaaaccacaggggaggcttactggaaaccctcttcctcttgggatttcatcactctcaaacggagggagatcccagggtacgcaggaaaggaccatggagatgtgggcaaagagcctgcggacctgttatgatagtgccggaggggaggttttcaaggcgccaccccggaggaaaatgcaaccctctcatcctaaggttcacagatgctggaaaaagaactacttgggatagtcctaagtctggggactcggctgcaccgagcagggaaagatccggtgacttattctccctgtacagacaaattactccctaagccaacaatcagtcgggccaaacatagtaatagcggaccagagatccccaacccattttcaagtccctaaacccctaccgttcctaaagctatcactcctacaccaggtgctgtcaccttctccccaccccagatgccctaaacatcgagataaccagagaccctccaggtaccagagatagattattacaattaatccaaggagtttaccaagccttaaatttttcagaccccaacaagactcaggaatgctggttatgcctagtttcccggccccatattatgaaggcgtggcaatactgggcaactactccaaccagacctcagcacctaccagttgtggagctgctatgcagcacaagctcacaatatctgaggtctcaggaaaggggctatgcataggcaggattcctcctcacatcaagaattatgtaaccaagtagagccattatctcaggacagccgataccttgttgccccttatggaacttattgggcttgcagtactgggttgactccctgtgtctctaccactgttctcaacaccaccattgacttttgtatattgatagaactttggcccaaagtcacataccaccaacctgaatatgtttacagcgtactagagaaatcaacccgatataagagggagccaatatcctttaccgtggccctattattaggaggaataacagtgggggcatagcagccggcatagggaccggaaccgttgccctacagggaattaatcattttaagcttctacaacaagccatgcacacggatatccaggtcctagaagagtcagtcagtgcactcgagaaatccttaacatcactctctgaggtggtcctgcaaaacagacgaggattagatttattatttttacaggaagggggctatgtgctgccctcaaggaagaatgctgcttttatgcagatcatacaggaatagttagggacagcatggccaaacttagggaaggctaaaacagaggcaacagctatttgagtctcaacaaggatggttcgaaggatggttcgctaagtccccctggttgactacccttatatccacgctcatgggacctctggttattctatttttgatcctcatatttggtccctgcattctgaacaaactgactcaattcatcagagaacgactatctgttgtacaggccttagtcttaactcaacaatatcatcagctaaagcaaatagatccagagtatccagagacctctgaatgaaagattccattcagttacaagagaaatgggggaatgaaagacccctgtcccttagccctttctttctcaagtttgtctcctcttcctcctgtcggcggcttccccgatccccacccccggtggccttccccgcccgccgagaacagcaccggtggccggccgagaagagcaccaggtggccagccgagaacgagcaccggtgggcggcacgaggcgagcaccaggtgggtcagcacgagaacaaacaccgagtgagccggcctggagccctgcccctgagcccccgcccgaagagaaacactccgtcccaaggtctccgccccaaggtcagccatcaggaaagggggggaattgagtctgctgtaccagacaccagaccttgagaatatgctgatctggaatggctctgtgtctcatttgaaccatccaatggaaatgattctgtatttcgcctcatttgaaagactctgtgtttcacctcatttgaataactctgtactttgcctcatttgaataaccctgtatagcgcctcatatacattgaccaatgggaatagctctgtataatgcctcattagaattatccaatagaatccctgctcctagcttgcgcctttttcctatataaggacccctttcccttggctcgggcgcttagccacacagaagctaagtcgccccggtaccgcgtctccaataaagcctcttgttttttgcatccagttcgtggcctcgctgattcctgggtgtgggtctccctctacgaaagtcctcttcggggtctttcacacaatctgagttttaagagtaggttctaagtgggtcgtgagctcaggctgagtctttgttgtccaggtcattcttgatttcttctcacatgctctccagatcttgtgtgtgttgtgtgtgtgtgtgtgtgtgtgtgtgtgtgtgtatgtgtgtgtgtgtgtgtgtgtgtgtgtgtgtgtgtgtgtgtgtgtgttaatggaaatttaattcatttggcaaaaatgcagtaaatcagtaacagcatagcagaaattacatagaaaacaaatatcgatacacatagtgtgaaacacacaactggttaaaatcatggatgtggtgagtaaaatccatctgaataaagtaaccaatgttaaaatgacttccttactttaaatgaaaaacctctgagattattaggcactataatctgtatcattataccacaatttgcagattttcaaaatgtgaagatttctacaactgaaccactttggagaaaatttctactgttggtgtagagacagtgactcaaggcttggatcatctcagaaggtttcagctttgatgattcgggactcacaagactcaataataagtaatcatcatgcttacagcttcctgctacagtgaatacctggaaaaggtacattctggtgggatacagacaggccagaaagggctgattccttcctcatatgaggagaccatgaaatgtgcattctgtgacacagggagctacaggaacacatgcagactgtttatgcccagaaaaatctgctcaagtgtcaggatcagtggctttagtggggtgttgaccgtaaagacatactctgctacccaaccagtcacagtaactaaaactcataactggaccatgaaacacactcatgctattaaacttgatgttcatgtattgcaatacaacccaagagcctggcatggtgtataccactgctttaggtataaacagcaaaatccccatctctaatgtaaagagcatgctagaaccacattctgggactgaccaaggtcagtgtcagtttcaggtgcccagaggcaggcaaagagtaaacaactagatcaatgttaataattcagctatatgtattcctttgtatgcattaattaatatagaaggcatacttaatatgtttaacctatagactctaatgaagatctatgacatacatgtcacaatgccagaaatgtgaatgaatttcattagaagtcttttgtgattttgttttgtttcttacctctgcagcaaaaatagttagctcattttcctgtcaaatattatcaacccaacagaaaagagataacaaagtagccaaagaaatttggttgcaccaagttttgtatgctaccatgggacagaattttcaaggaattccttagaaattgaacttggatggcatatattgccaaaaggcacagtgggggacctgcaagccaaaatatacatgaataagagaaaggaatacacagaaagggtgtgtctttctggagagtgtctggtggcttccacctctacagcctcagagggagctttaggtgcagccccaagctggtaaaagttcctgatcagaaagctccctgatccaatctccgatgactccactcactgagcgtacacagtatggccaggccatgttcaaagggagaattctcacccacaacttgaagcaacacatccaggaaggcccctcttctcactgaccagtatcaactcccagcgtctagccaacacatactgaaagcctttcctcatttctgtcacaaagcttccaaaatgcacatgttggtggaagacttagctatagcatgatctgaatagagaccttttattttccacacaattggtgtgtgtgtgtgtgtgtgtgtgtgtgtgtgtgtgtgtgtgtgtgtgtacaggttgagatgagaggtcaaccttgattgtcagtcattcctcaggatctgtctagtctctcacaagcacctagggttcattgataaggtctgactgactttccaggagcaccggtgacctggctttctccatctcctcagaaatagaagtacaagtgcacaccaccacatggctgttcacatgggtgctaggcatcaaactcaggttcaaattttactgactgagccatctttcaagcctctgatcggtctatgtttagtttcagaagctccaagacaacaatgtgcactttatagagcttcatgtggaagtctagagatacattgaaaagtgcccggtaacttctaacacagcccaaggcccagtgggaacagaagtaagctcactttccctgagagtggagtgtagaagttgtccacttccaggtgtttgcttggcaactgtgaggcctgcacagagttgactgacatcctactcaggaaggcatcaaaaggaagagtctggctacctgcccattcactaacatagcttggagttccctggtttacactttaaatgtttcagcctgaacagtgtactacacatataacaggtgagccctgggaccagctactcaaaatctgcagtatcaaggaagaataccactccaactaagagtagtatttgagacactatccaggaagccttgcttcagacatgactactacaccttcaataatctcatcaatggtgttaggattgttcttcaatggagaagctcttacaattaatggtttttatttttattatttatgtgtgtgtgtgttgcctttttgcgtgtatatgcctggtgctcttgaatgtcctgatgcctgaagagatcagaagaggacattggatcccctgaaactggagttatgggtagttgtaagccaccttgtaggtgctgggaactgaaatcaggtcctctgtaagaacagccgagtgttcttaaaggatgagtcatctctccagcctcacaattaatcagtttggtagaatacttgcctgccatacgtaagagatctggtgatgatccctaacactcctccccacccctctcctctctctgtctcttgtctctctctcttacacaccacacttttaattcttaaaatgagtcatatttctgttacatggcttgcttaggaactgcaggaacatttacccttatgtgatatgggaacatgggataggaattcatggacatagcaaatcacacttttaaaaattcttgttctagggctccacctgagatcataacacttcaattcaagagatcacctattctgactagaatagtatacattcagtgaactcatcatccaaggccaccgagcttaaaaaaagcagatcatctagcacttgagtgatggcccactgcaagctgtttgtgtggca

SEQ ID NO: 19 - DNA, 12622 bp, Cricetulus griseus
  misc_feature: 000351F-017-011-31837_Type_C
gaataaatctaaaagccaagaaaatctaaaaatcctttgaaggaagagaataaaactgttcaagatcaagtcaggcattggtggtgcacgcctttaatccagcacttgggaggcagaggcaggcagatcctgtgagttcgagaccacctggtctacaagagctagttccaggacagcctccaaagccacagagaaaccctgtcacgaaaaacaaaacaaaacaaaatgttcaagacctgaaaatagaaatagaagcaataaagaaaacatactgaaggaattctggaaatggaaaatctaggtaagtgaatagcatcaccaacagaatacaagagatggaagaaaaaaactcatgtgttgaagatacaatagaagaaattgatatactggtcaaagaaaatattaaatctgaaaaattccagacacaaacaccaggaaatggaaatctggaacataaaatatcgaggaaatctaggacactatgaaaagaccaacctaagaataataggaataaaagaagtcaagaatcccagctcaaaggcaaagaaaatatttttaacaagatcctagaagaaaatcttcctaacctaaagtaggagatgcctacaaaatacaacaggtttacagaacaccaaatagatgggaccagaaaagaaaattgcttcaccacataataatcaaaacactaaacacagaaaagaaagaaagaatagtaaaagctgcaagggaaagggctaagtaatatataaaggcagaccttttagaaatacacctgaattgtcaatggaggttctaaagactgaagtctgcccagatgtgctgcagactctaagaaactacagatgccagaaccagattactataccagaaatattttcaatcaccatagatggagaaataagatattccatgataaagtcaatttaaataaatccacaaatccagtcctatagaaggtactagaaggaaaactccaacccaagaagataactataccatgaaacacagaaactaaataatctcatcaaaaagaaggaatgcacacacacacacacacacacacacacacacacacacacacacacacacacactgacactgcctctgcactgcactgaaagacccccggagaggttctttcgtggaagaagacccacacccaggaatcagcgaaaccacgaactttggatgtaagaacaagaggatttattaataacacggatatccggggcttagcttctgttttgccaagcccgagccccggaaggagggtccttttataagggaaaacaggcatagtacagaagcgaaaagcatcaaatcagcattttctcaaggtttaatagccaggcgaaatggccttatctcctgccggtccagatggctggccttgggaccgagcgatccggcgggggttggccttgcgtggggcggggagtgaggccagcagctgcagagatcaaaaggtgctaaggagggaagtgctaaggagggaaagatgtattcgcgcgccccgccagctgccgaccagagtagaaaggctacagcccggggtgggggtctttcatcccccatttctcttgttactgaatggaatctttcattcagaggtctctggatattctggatctatctgctttagctggtggtattgttgagttaaaactaaggtttgtatggcagataatcgttctctaatgaattgagttatcttgttcagaatgcagggaccaagtataaggattaataatagaataatcagaggtcccatgagagtagataccagagtagtgatccagggggacttagtgaaccatccttcgaaccatccttgttgagactcaaatagctgttgcctctgtttagcctttcctaagtttggccatgctatccttactattcctgtatgatctgcataaaagcagcattcttccttgagggcagcacatagcccccttcctgtaaaaataataaatctaatcctcgtctgttctgcaggaccacctcagagagtgatgttaaggatttttcgagtgcactgactgactcttccagtacctggatgtccgtgtgcatggcttgttgtagaagcttaaaatgattaattccctgtagggcaacggttccggtccctatgccggctgctatgcccccactgttattcctcctaataatagggccacggtaaggatattggctccctcttatatcgggttatttccctagtacgctgtaaacatattcaggttggtggtatgtgactttgggccaaagttctatcaatatacaaaaatcaatggtggtgttgagaacagtggtagagacacagggagtcaacccagtactgcaagcccaataagttccataaggggcaacaaggtatcggctgtcctgagataatggctctacttggttacataattcttgatgtgagggagggatcctgcctatgcatagcccctttcctgagacctcagatattgtgagcttgtgctgcatagcagctccacaactggtaggtgctgaagtctggttggagtagttgcccagtattgccacgccttcataatatgggggccgggaaactaggcataaccagcattcctgagtcttgttggggtctgaaaaatttaaggcttggtaaactccttggattaattgtaataatctatctccggtacctggagggtctctggttaactcgatgtttagggcgtctggggtggggagaagatgacagcatctggggtaggagtgatagctttaggaacggtagggggttcagggacttggaattgggttggggccctctggtccgctattactgtgtttggcccgactgattgttggcttaggggagtatttgtctgtacagggagaataaggtcaccggatctttccctgctcggtgcagccggagtccccagactttaggactatcccaagtagtcctttttccagcatctgtgaaccttaggatgagagggttgcattttcctccggggtggcgccttgaaaacctcccctccggcactatcataacagggtccgcaggctctttgcccacatctccatggtcctttccctgcgtaccctgggatctccctccgtttgagagtgatgaaatcccaagaggaagagggtttccagtaagcctcccctgtggtttcacatccccagcttgcacagtacccttcctgtggcccccgcagtcatgagttggtctatggccaggacaaacataaaaatccttgcttcttgtcctatccttccccagggtggtggcatccgtaccctgggaatggttcctggtcagagggatcccaatcagaccctactaagtcacataggtcgacatagagaggagggaacccatcatgtagagtccctatataagtgctgaggttggctatttccccggtccctagattggctattttccaggtgatattataaaccctgtgggggttagttgctacatggggggtaaacagacaaccaatcaacaggagggtgtacgagagagtcttatctttaaaggattttgagtgcggtgtacggtccattctgatgtagtggctgaatcggtgggtcgggctggctttacatgtgaagcatgtatccaagcggcaatgccgtctactttgagtgcggtgggagtggtaagcaaaacgatgtagggtcccttccagtggggttcaaggttcttggcctggtgacgccggacccaaacggtgtccccgatctggtaggaatggggatggtgggatggtccctctggtctttataagcttgagcaaggggtttccaaatctcccgttgtactaattgtagtgcctgtaaatgagcttggagagaaggggagttagcaaaatctgagacatcttgatcaagaaagttaatgataggagtaggtactccatgcaggatttcaaaaggtgtgagcccatgtggcccaggggtgttgcgagcacgatagagggctagggggagtaggaggacccagtctctagtgccagttgcaagcgacaattttgttaaagtctccttgattgttctattcatcctttctacctgtcctgaactttggggtctataagcacaatgtaatttccaatcaatccccaacaaggtggccactgactgacttatctgggaggtgaaggcgggcccattatctgttcccagtacctgaggcatcccataacgtggaaagatttcttcaagcaatttcttagtaacgatcttggctgtttcatgtttagtaggaaaggcttcaacccatcctgaaaaggtgtctacaaaaactaatagatacttgtatccatatttacctggtttaatctcagtgaaatctatctcccaatgtgttccgggccggtagcctctgacccggttcccggaggaagcttcagtctggatgcgttgactcgggcgcacgcatcacattgttcagttacctgttttaaaatgtcattcttccccaaaaggaaattgaattcctcttctcgttcgaggagctcccgcatcttcttggagctcagatgtgtcatcttatgtaaaaatgtcactaggtacttggttacccggtaggggatgacagtctttccttcataagtccaatccccgtttggttcctttgtggctcccaatttctctagggtttggatgtctctttgttcatagtcccatgagggggagggacggtcgttggtgggctccagagccaggaggctagtggtgtctgtggtcagggccgcctctcgggcagccaagtctgccctccgattgcccctggcttcaagagaatcccctttttgatgtccgggcaatgtatgatacttaaggcggagggcagatgaagagctcttaggagagcgaggatttcctccttattcttaatgtctttcccttcggaggtaagcagccccctccgtctgtaaatttctccatgtatatgggcagtggcaaaggcataacgactgtctgtgtacacggtgagcctcttaccttctgccaattttagagcctgcgtgaggcaatgagctctgccctctgcgcagaggttccgggagggagtgcctgagcccagacaacctgattctctgtggtgactgctgcgcccgcccttcgttttccttgatgcaaaaagctgcttccatccgtgaaccagatgtggtccgggctgggtagaggctggtcagtcaggtcgggtctcgccccatgggcctcagccagtacctgtaagcaattatgagcctctggctcccctgggagagggagcaaggtggctggattcagagtcaccagaggcccaaagtggacccggtctttgtccaacagcatagtctggtaatgagtcattcgggcattagtgagccatctgtctggaggctgtctgatgactgcctcaaccgcatgaggggcatgtatggtcaaaggctgccccaaggttagcttgtaagaatcttttactagcagggctatggcggcaatcattcggagacagggtggccatcccgatgctacaggatccaatttcttggagaggtatgcagtgggccttctccaaggccccagtttttgggtgaggactcccttggcatagcctcctttctcatctataaagagttcaaatggcttagctaagtccggtagacccagggctggtgattcaaggagagcctttttgatgttggtgaaggctttcttttggggctcttcccatttaaaagcaacccctggccgagtgagggggtagaggggagccgccatttcggcaaacccggggatccataggcggcagaagcctgccgttcccaagaactctctcagttggcggggattttgtggggtgggaatgtctaaaatggcacgcatacgggcttccgttagccatcgccgtccatcctttatcttgtatcctaaataggtcacctgtttctgacatatctgggccttcttggcggatgcccggtatcctaggctcccaagagtctggaggagggctcgagtgccttccttgcattcttccttggttttggctgccaggaggatgtcatctacatactgtaagaggattagcgcggggtaccggacccggaatcctgccagatcctgatgtaaagcttcatcaaaaagggtggggctgttcttaaacccttgaggtaacctagtccaagttagctgcccagagattccaaggtcggcatcctgccattcaaaggcaaatataggttggctggtgggatgcaggcggaggcagaagaaggcatctttaagatctaagactgtataccaggtatagttgggtggcaatccgctcagcaaattataagggttggggacagtagggtgtatgtcctctatccttttattgacctccctcagatcctgtactggcctatagtcattggttcctggcttcctgacgggcaacaaaggtgtattccaaggagatttacagggcaccagaatcccttgttcaagtaaccttttaatatgtggccgaattccttcgcgggcttctttaggcattggatactgtctgacggatgcaggaaggatggcagcttttaaggttactataattggggcctggttgatggcaaggcccattcctcctgtttcagcccaggcttggggaaattccgcaagccagtgatcaagggtttcctgattgttcgaattagtcctcttttcatggagtctgtattcatcttctatggacattgtcaagatggtaaggggaattccctctggccctgtgactttgacttctgacctctcaaagtgtatttgagctcttaatttggtcaataggtcccgtcctagtaaggggtaggggcaatctggcacatggaggaaagaatgcataaccttaccagtcgcgagctggagctgccgatttgtagtccaatggtactgctttccgcctgtagctccctgtacccatgcagtccggtgactcaggggtccgggtgaccgattcagaacggaatgttgtgcccctgtatctactaggaaggtgaccggatgccccccgacttgcagtgttatcctgggctcaggggggggctcctggccctgacttctctaatcttctaggtttagaaggtcagaggttcttggccgtggaggcttggcccgggggttcttagggcattcttttgcccaatgtcctttttctttgcagtaagcgcattgatccctgtccaggtggggcccccttctgccatccctctgcctattctgtctctgacctgtcactactgtggccaatagcctgctcatctctctattccgcttacggtctcttgcagcctctttctcctctgcttcttttctcagtctttcctctctctcctgggcttcctttctccaacgttcttctctttctgtctgtgtctctctcttattaaaaatacgttctgcttcctttaacaagtcttggagcgtatatccttgtaaattttctagcctttggagcttgtttcttatgtccggggctgactgccaaataaaggacatagaaacgctggtggcctgccctggatcttctggatcatagggagtatacattctatatgctccttttagtctctctagaaaagtggcgggcgattcctccgcccctgtattacctgctttacctgggccaaattggtaggtcggcgtcctgcccccggagacccgcgaccagcaactggcgatagagacgcagatgggtcctacctgcttcagtagtaaaatcccattcaggtctttcaagagggcaagccgcatcgatttcattgggcagctgggtgggctgcccgttggctcctgggacattctttcgtgcctctagtagaacacgctgtttctcctccgaggttaaaagaacctgcaaaatctgctcacaatcatcccaggtaggttggtgagttgtgagtaccgactctatgagagatgtcagcctcacagggtctgcagagaaggaaggattattatttttccagttatagaggtccgatgctgagaatggccaatattgaggttggccattcggtccagttcggaggggcaaagcggttgaatctggagcgggctgctccctgcgacctcttagtcggtgagccattggggaagggtcaggtgggggagctgaagccgtctctgccgagacggcgggggccgcttctgcctctggcgggggcggcagcgccgggtatggagggggttcttcagtcaataggtcagccaacaggttttctcccggggaagcacctcaggcgtcttcttctcttcagctttagcgtcctttaccgctgtagggtaaaggctggagtggggagggagctggggattggaaagggttggggttgagggaatgggccggttggagcggttagagggaggaaggagggggagcccttggggtgtaggaaaggacgtatccaggggggggtcctgaaccaaggcctcccaagtgactatgtatgccacctggtcgggatgcccctgtggtccaggatccatagtcttctctttcacctgaaatatagtttggaggttaaaggtaccatctcgcggccagccaacgtcaaaggctggccattctgaggaacaaagagtaatccatttgcgtttacgcacatcaacagattggttatgagcataatcctgcacatctctccagtgtgagagtgttagggacaagggagtggtgatagtttgtcccatagtttctagacttagtagaacacagataatggttatgatacaaagactaagcaaaacagaacaggctttcgctgcgcggtttcgacagagagtcgatagtaagatttagaaggcgttgaagaagggggccagccttcttcgtcccggtaaggattgcaaatccttcaagccgaggaccctctccaaagaggttgggaagctgtccagtcatagatctcagctcaaaaacaaaagtacaaaggtgagcccactaatgcttcaatcgctaccaggacgtctcctggagccagggtcgggagtccgaacccgtcaaatacaaatgtacgcagctgtgtactaccaacgcgggcgcgcagttcatgaaatggtttcagacagaaagacaggaccagacgagacaagacagtggatccacataacacttacctcccaaatgtgggtcggtggtccctgggcgggggtctcgatcccggacgagccccaaatgaaagaccccggagaggttctttcgtggaagaagacccacacccaggaatcagcgaaaccacgaactttggatgtaagaacaagaggatttattaataacacggatatccggggcttagcttctgttttgccaagcccgagccccggaaggagggtccttttataagggaaaacaggcatagtacagaagcgaaaagcatcaaatcagcattttctcaaggtttaatagccaggcgaaatggccttatctcctgccggtccagatggctggccttgggaccgagcgatccggcgggggttggccttgcgtggggcggggagtgaggccagcagctgcagagatcaaaaggtgctaaggagggaagtgctaaggagggaaagatgtattcgcgcgccccgccagctgccgaccagagtagaaaggctacagcccggggtgggggtctttcacacaccaccaataacaaaataacaggaattagccatcactggcaattgatatctatcaatatcaatggactcaattccccaataaaaagatacaaactaacagaatggatgtgataacaggatccatccttctgatgcatacaagaaacacaattcaatatcaaagatagacattacctcagagtaaaggcttggaaaaatatttcccaagcaaatggacctaagaaacaaactgatgtagctattctaatatctaataaaatagtcttcaaaccaagggcacttcatattcatcaaagaaaaagtctgccaagatctttaactcttaacatctatgcaccaacagttgctggatgaagtgtcattagggaatcacttcattgaacttgtgggagtggctaacccatgactggtctaactttaggtccccatatgaggggcagcccatgcctgacactgcctggatggccaggaaccagagagagaacagctcagagacccagagtagaatcaatcacaactgtcagaaaagataaaatggtcaatgaagtgattcctagtattattctgctgtactcatagatcaatgtgtagcctagtcatcatcagagaggcttcatcaggcaactgttgggaacagatacagagacccacagccaaacaggaggagctcagaaaccctcagaagaaggggaagaaggattgtaggagccagagaagtcaaggacatgacaagaactcattccactaaatcaactaagcaagactctcataggggctcacagagactgaatcaacaaacatggacggaccctctatgggtctgagctaggtcctttacatatatgtaatgggtgtgttgcttggtgtttttgtggaacacctaacagtgggagtgaggactgtctttgactcttttgcctgcttttgggaccctttgcctccttttggtttccttttccaaccttgatgtgattgtgcccagtcttactgtaatttgttatgccaggttcagtggatatccctgggaagtctgctctctctttttttaaaaaaagggaaatggaggaggagtggatcagagggagaaggaagggactgggaggagtggagggaagggaaattgtgatcgagatgtaatgtatgagaggggaatttaaaaaaagacatgagctatctcaagtggtgtattggcaatctggaagtactgcacagggatgatcagcattcacaccttactggtaaaggcctgaatttgcaaagtattgccctgaaggtacagtcaaaagtgtaaacagtgtatgtggttcaacttgtaaaggacaccctattgaccataccagcattaagtaaggcccagaggtcagaaaagcatttgaaacttgattttggaagtacatcctagaaggatgaccagcctttatgacctgacctcacttaggaatgctaatggatataacaagcatctaggaggtcagaaagagaaactgagagacttatgagtgtaacccacaaacagaacacaatgcatccattcatcataaccccaaccaactctccgctctccacatttatttctcattcatttaatatccaaagtcagtttagcaggtaaactctgtccttatctctcttatctgaaggtattaatggggattgatcctggtgtccgttccagatactagtataacaggaaaattatgttaatcccaaaatgggaggagaaagaccaccaagcaattaatcaaagcaagcttttttatttgtgtacacaggctgcctcccactaagatggggttcaatagatcagaactggaattgaggaagacaaggcttttatggggctcaggagtagggagtttccaaatagttagagttacaggaacagaagtaacagatggtcaaaacaaggtaatcacaacaggtagtcataataaaatagtcataggtggtcctataaccttttttaaaaaaggtaaagttaaaagagggttataaacaacttttgaaacaaagacatgattgccattcctgaaagaagcagtaaagaaccatttgttgttaggattgcaggtgggatatagcccaatccttgaaaaacaggtttaatcataaacagggatgaacttattttgtctttactataagatggcttttaagcctaagatggaggcagggtggttcttcactagctcaaccctactagaatctagtctgtcttgaaaattgctagcatttacactaacagaataaggggaatcaaaaaattatcaactaatgtcctagcatcttatatccacattgcaacacaaaaccaaaagaaacatgacaaacaaaagcagaatgactctgaaaacttactaattccacagtaatggccaacagtgagaacaagttagatgaaataccaaagaatttaaaaggatgattatacaagtgttcagatgaattaacaagaacacaaactcttgaatgaatttcaagagatcttaaacaaacagatgaaggaaataacatgaaatgaaaagagaagtgaaactggaattcaacaatgacaaatactgaagaaaaaccaaagtgaaatactggaaatcaaaaattcaaaaaagaagctgggcaggggtggcgcacacctttaattccagcactggggaggcagaggcaggcggatctctgtgagttcaagtccaacctaatctatagagtgagttccagaacagccacgactgtttcacaaagaaaccctgtctcgaaaaaacaacaacaacagcaaattgaaaaagaaaatgaaaagctcttggaatgcctcaccagcatgtgtcaaggggatgacagaaatattatgactcaaggcatggcagaggaattggaatagaaagacaatagcaaagttaataaactactaatgaaacactcaaaatttaaccacaacacattaaaaaatcaaagaatcatggacaaa

SEQ ID NO: 20 - DNA, 13483 bp, Cricetulus griseus
  misc_feature: 000519F-009-011-25000_Type_C
ttattgagaacactcctagttaaaaatcccttggaaaaaactattactgcagtagcattaacaacaccagaagagatggtcattgacttagagagtgacttgtctggagattgtaagtgactgccatcttaaaagaagagctgttgcccaatggtgtacatttcctttctcaaggacagtgaaggacttattctttctattcaatgtttccttttttatttaaattaaaagacaagtttgttttacatatcaatcccagttccctctccttcccttcctcccctgacctcaccgatcctatcacatttctgcttcccagggagggtgggccttcaatggggcatcttcaaagttttcatatcatttggagaagggcctaggccctccccatgtttctaggctgagagaatgggctcccaaaatccattcttgtactagagataaatactgatccactaaagaggccccatagattgcccagacctcctaactgacatccacattcaaggggtgtggaacagtcctatgctgatttcccagttatcaatctggtgtccatgagatcccccttgttcagttcagctgtttctgtgggtctctccagcctggtcttgaccctttgctcatcactcctccctctctgcaagtggattacaggagttagtctcattgcttagctgtgggtgtctgcttctgcttctatcagatactggatgaaggctctaggacatattcttaaaacctggagatcccacactgagtgcacttgaaagttgcaagaggtggcttacaagagaaataacttctgtctttccagatttgcatatccttatctgcattgtgcttcccctattgcccagggagcctttgaaacacctcaccttggagcactatcagctttaagctgtacccagcatcacactaggcaatgctgcacctccagactccaagccaaaattctgcctttttcacccttcacaacttcagtttaatgtcatgtttcatgtatgacatggattccacaggggctgtctaaatgggacaaattgacaagacccagaaatctaagactgtgatgagaggtaacgtccttctgtctacatcacactctactagaccagtacaaagtgggctacttctcttgaaattactttaattccatggtgaaatacaagcagctggtgttcttcaatgtagccatgccagatgagcaaagagctgccatatagctgcagttcctccttctctgcttctacctttcccttcattattgatgacttccagaaagttttgcttctgtttgtctccagaaagttctggctttgagtaaacatttggagtcagttctttgagaaattctttgcacactgaaccacaatccatgagaaagggagtgcaccacaacttgacgactgtacttcaacagtcacactttgtcctggcaataaaaggaaatgttagtgctcatccaatactctgtcctctcataactctcaggtaggcattctactgaatttttgcctaagtgacttgaaagtctctttcctccagcaatttaccttactaagcatctttttataactttcaaattgatgtgttgtttaaggttgaaatatttgcacactttaggaaagctctgttggcatattgctacaataaaaaatactgtaaaacatttctctataaaacaaagatttagaggaagctgttgctcttaaaatttgctgatcaggattacatgtaggaacagaatgcttaatggttgtgaaatccaactctatctaaaataaagatacaaaatatatttttggaaaacaaaacacaaaaatgtttttagtagttgttgaaattaggtttgaaagacccccaccctgggctgtagcctttctactctggtcggcagctggcggggcgcgcgaatacatctttcctccttagcacttcctccttagcaccttttgatctctgcagctgctggctctcactccccgccccacgcaaggccaacccccgccggatcgctcgtcccaaggccagccatctggaccggcaggagataaggccatttcgcctggctattaaaccttgagaaaatgctgatttgatgcttttcgcttctgtactatgcctgttttcccttataaaaggaccctccttccggggctcggggcttggcaaaacagaagctaagccccggatatccgtgttattaataaatcctcttgttcttacatccaaagttcgtggtttcgctgattcctgggtgtgggtcttcttcacgaaagaacctctccgggggtctttcatttggggctcgtccgggatcgagacccccgcccagggaccaccgacccacatttgggaggtaagtgttatgtggatccactgtcttgtctcgtctggtcctgtctttctgtctgaaaccatttcatgaactgcgcgcccgcgttgtagtacacagctgcgtacatttgtatttgacgggttcggactcccgaccctggctccaggagacgtcctggtagcgattgaagcattagtgggctcactttgtacttttgttttgagctgagatctatgactggacagcttcccaacctctttggagaggtcctcggcttgaggatttgcaatccttaccgggacgaagaaggctggccccttcttcaacccttctaaatcttactatcgactctctgtcgaaaccgcgcagcgaaagcctgttctgttttgcttagtctttgtatcataaccattatctgtgttctactaagtctagaaactatgggacaaactatcaccactccttgtccctaacactctcacactggagagatgtgcaggattatgctcataaccaatctgttgatgtgcgtaaacgcaaatggattactctttgttctcagaatggccacctttgacgtggctggccgcgagatggtacctttaacctccaaactatatttcaggtgaaagagaagactatggatcctggaccacagggcatcccgaccaggtggcatacatagtcacttgggaggcttggttcaggacccccccctggatacgtcctttcctacacccaagggctcccctcccttcctccctctaaccgctccaaccgcccattccctcaaccccaccctttccaatccccagctccctccccactccagcctttaccctacagcggtaaagacgctaaagctgaagaaaagaagaccctgaggtcttcccccgggagaaacctgttggtgacctattgactgagaacccccccatacccgccgccgcccccgccagaggcagaagcggccccgccgtctcggcagagacggctcagctccccactgacccttcccaatggctcacgactaagaggtcgcagggagcagcccgctccagattcaaccctttgcccctccgaactggacgaatggccaacctcatattggccattctcagcatcggacctctataactggaaaaataataatccttcttctctgcagaccctgtgaggctgacatctctcatagagtcggtcctcacaactcaccaacctacctgggatgattgtgagcagattttgcaggtctcttaacctcggaggagaaacagcgtgtctactagaagcacgaaagaatgtcccaggagccaacgggcagcccacccagctgcccaatgaaatcgatgcggcttgccctcttgaaagacctgaatgggattttactactgaagcaggtaggacccatctgcgtctctatcgccagttgcttgtagcgggtctccgggggcaggacgccgacccaccaatttggcccaggtaaagcaggtgatacaggggcggaggaatcgcccgccacttttctagagagactaaaagagcatatagaatgtatactccctatgatccggaagatccaggcaggccaccagcgtttctatgtcctttatttggcagtcagccccggacataagaaacaagctcaaaggctagaaaatttacaggatatacctccaagacttgttaaggaagcagaacgtatttttaataagagagagacacagacagaaagagaagaacgtggagaaggaacccaggagagagaggaaagactgagaaagaagcagaggaaaagaggctgcagagaccgtaagcggaatagagaatgagcaggctattggccacagtagtgacaggtcagagacagaataggcagagggatgcagaagggggccccacctggacagggatcaatgtgcttactgcaaagaaaaaggacattgggcaagagaatgccctaagaaccccgggccaagcctccacggccaagacctctgacctcctaaacctagaagattagagaagtcagggccaggagccccccctgagcccaggataacactgcaagtcgggggcatccggtcaccttcctagtgatacaggggcacaacattccgttctgaatcggtcacccggaccctgagtcaccggactgcatgggtacagggagctacaggcggaaagcagtaccattggacacagatcggcagctccagctcgcgaccggtaaggtcatgcattctttcctccatgtgccagattgccctaccccttactaggacgggacctattgaccaaattaaaagctcaaatacactttgagaggtcagaagtcaaagtcacagggccagaggaattccccttaccatcttgacaatgtccatagaagatgaatatagactccatgaaaagagactaactcgaacaatcaggaaacccttgaccactggctcgcggaatttccccaagcctgggctgaaacaggaggaatgggccttgccataaccaggccccaattatagtaaccttaaaagctgccatccttcctgcatccgtcagcagtaccaatgcctaaagaagcccgcgaaggaattcggccacatattaaaaggttacttgaacaagggattctggtgccctgtaaatctccttggaatacaccttgttgcccgttaggaagccaggaacaatgactataggccagtgcaggatctgaggaagtcaataaaaggatagaggacatacaccctactgtccccaacccttacaatttgctgagggattgccacctaactatacctggtatacagtcttagatcttaaagatgcttcttctgcctccgcctgcatcccaccagccacctatatttgcctttgaatggcaggacctgaccttggaatctctgggcagctacttggactaggttacctcaagggtttaagaacagcccaccctttttgatgaagctttacatcaggacctggcaggattccgggttcggtaccccgcctaatcctcttacaatatgtagatgacatcctctggcagccaaaaccaggggaatgtaaggaggcactcaagccctcctccagactcttgggagcctagggtaccgagcatccgccaagaaggcccagatatgtcagaaacaggtgacctatttaggatacaagataaaggatggacggcgatggctaacggaagcccgtatgcgagccatcttagacattcccacccacaaaatcccgccaactgagagagttcttgggaacggcaggcttctgccgcctatggatcccgggtttgccgaaatggcggctcccctctacccctcactcggccagggttgcttttaaatgggaagagccccaaaagaaagccttcaccacatcaaaaaggctctccttgaatcaccagccctgggtctaccggacttagctaagccatttgaactctttgtagatgagaaagagggctatgcaagggagtcctcacccaaaaactggggccttggagaaggcccactgcatacctctccaagaaattggatcctgtggcatcgggatggccaccctgcctcgaatgattgccgcatagccctgctggtaaaagattctcacaagctaaccttggggcagcctttgaccatacatgcccctcatgcagttgaggcagtcatcagacagcctccagatagatggcttactaatgcccgaatgactcattaccagactatgctgttggacaaagaccgggtccacttcgggcctttggtgactctgaacccagccaccctgctcccctccctgggagccgaggctcatgattgcttacaggtactggccgaggcccatgggcgagacccgacctgactgaccagcctctacccagcccggaccacatctggttcacggatggaagcagctttttgcatcaaggagaacgagggctggcgcggcagtcacctcagagatcaggtcgtctggcccaggcactccctcctggaacctccgcgcagaggcagagctcattgcactcacgcaggctctaaaattggcagaaggtaagaggctcaccgtgtatacagacagtcgttatgccttgccactgcccatatacatggagaaatttacagacggaggggctgcttacctccgaaggaaagacattaaaaataaggaggaaatcctcgctctcctaaggctcttcatctgccccgccttaagtatcatacattgccccggacaccaaaaaggggattcttttgaagcaaggggcaatcgaagggcagacttggctgcccgagaggcggccctgaccacagacaccactaacctcctggctctagagcccaccaacgaccgtccctcccctcatgggactatgaacaaagagacatccaaaccctagagaaattgggagccacaaaggaaccaaacgggattggacttatgaaggaaagactgtcatcccctaccgggtaaccaagtacctagtgacatttttacataagatgacacatctgagctccaagaagatgcgggagctcctcgaacgagaagaggaattcaatttccttttggggaagaatgatattctaaaacaggtaactgaacaatgtgatgcgtgcgcccgagtcaacgcatccagactgaagcttcctcccggaaccgggtcagaggctaccggcccggaacacattgggagatagattttactgagattaaaccaggtaaatatggatacaatatctattagtttttgtagacaccttttcaggatgggttgaagccttccctactaaacatgaaacagccaagatcgttactaagaaattgcttgaagaaatcttccccgttatggatgcctcaggtactgggaacagacaatgggcccgccttcgtctcccaggtaagtcagtcagtggccaccttgttggggattgattggaaattacattgtgcttatagaccccaaagttcaggacaggtagaaaggatgaatagaacaatcaaggagactttaacaaaattgtcgcttgcaactggcactagagactgggtcctcctactcccctagcactctaccgtgctcgtaataccctggaccacatgggctcacacctttgagatcctgcatggagtacctaccctatcattaactttcttgatcaagatgtctcagattttgctaactccccttctctccaagctcatttacaggccctccaactagtacaacggaggtctggaaaccccttgctcaagcttataaagaccagagggaccatcctgccattccccattcctaccagatcggggacacagtctgggtcggcgtcaccagaccaagaacctcgaaccccgctggaagggaccctacatcgttttgcttaccaccccaccgcactcaaagtagacggcattgcagcttggatacatgctctcatgtaaaccagcccgaccaccgattcagccactgcatcagaatggaccgcaccgcactcaaaatcctttaaagataagactctctcgtacaccctcctattgattgtttgtctgtttacccccatgtagcaaccaccccacaggttataattcactggaaatagccaacagggacgggaatagccaacccagccttatatgggactctacacgatggttcctccttctatgtcgactatgtgacttagtggtctgattggatccttgccagaaccattccaggtacgagagaccaccctgggggaaggaagacaacgacaaggattttatgtctgcccggcataacaactcagacttggggggcaaggaatttctgtgcagctggggatgtgaaacacagggatgcttactggaacctctcctcttgggacatattcagaggaatccagggagcagaaaggcaggaatgtggctaactgcacctgttatgtaccagggatttaagcgcaccgaggaaatgcaacctcatcctaaggtacagatgctggaaagactactggatagctagttggggctcgctgtaccgcaggaagaccggtccttattctcgacagacaaattactcaccaaaacatgggccaaacccagttagcgaccagaaccccaccaattcaagtcctgcccctacctctaactcactcctacccagtctgcactctcccctcccagagcctaactcagttaaccagaccctccaggacagggaagattattaaataatcaagggttacttgcctcaattttcagaccccaacaagactcaggaatgctggtatgcctagtttcccgcccccatattatgaaggcgtagcagtactgggcaactactctaaccagacctcagccctaccattgtggagctgccatgcagcacaagctcacatatctgaggtctcaggaaagggctatgcataggcaggttcctccctcacatcaagattatgtaaccaatagagccattatctcaggacagccgataccttgttgccccttatggaacttattgggcttgcagtactggttgactccctgtgtctctaccactgtctcaataccaccattgacttttgtatattgatagaactttggcccaaagtcacatacaccaacctgaatatgtttacagcgtactaggaaatcaacccgatatagagggagccaatatccttaccgtggccctattattaggaggataacatgggggcatagcagctggcatagggaccggtacgttgccttacaggaattaatcatttaagcttctacaacaagccatgcacacgatatccaggtcctagaagagtcagtcagtgcactcgagaaatccttaacatcactctctgaggtagtcctgcagaacagacgaggttagatttattattttacaggaagggggctatgtgctgccctcaaggaagaatgctgcttttatgcagacatacaggaatagttaggacagcatggccaaacttagagaaagacttaacagaggcaacagctatttgagtctcaacaggatggttcgaaggatggttcgctaagtcccctggtcactactctgtatctactctcatgggacctctgattattctattttaatccttatacttggtccctgcattctgaacaagataactcaattcatcagagaacgattatctgccatacaaccttagtttaactcaacaataccaccagctaaagcagatagatccagaatatccagagacctctgaatgaaagattccattcagttacaagagaaatgggggatgaaagacccccccccggcttagcctttctactctggtcggcagctggcggggcgcgcgaatacatcttcctcctagcacttcctcctagcacctttgatctctgcagctgctggctctcactccccgcccacgcaaggccaaccccgccggatcgctcgtcccaaggccagccatctggaccggcaggagataaggccatttcgcctggctattaaaccttgagaaaatgctgatttgatgcttttcgcttctgtactatgcctgttttcccttataaaaggaccctccttccggggctcggggcttggcaaaacagaagctaagccccggatatccgtgttattaataaatcctcttgttcttacatccaaagttcgtggtttcgctgattcctgggtgtgggtcttcttccacgaaagaacctctccgggggtctttcagttgtcacctttagattactggtagagaaaaggtcacatttcagaaaaagaaaatgaatacattagcacaagagactgacatataatatctcattgtaaatcgctaatttataaggagaattctgaaggggtttttttgccattggctcagctgaagcttgtagatatatttaactattaaatataatgtcactgacatactgaaatgacattggatttcagtaacgagatgcccagttgatgtcaagagacttagttagttttaaaagaaaagattgcagatattcagtcttcacttgcagcctaggtgtccaagctgcaggagtttgtcatacatatatagttagagctaagcagaagaaatgatgccccaatggcacagaatccaaagaagaaccacattatgttctactaactttaaaaattcattgtttttcttaaagttgtcttctctaaaatatactctaagatatatcaaaaagctaaggaatattttcattaaacagatcccaacacaacaaaaggaaatgaacatttcagggctatcagcagaagttccagcacagaggtggtgctgtctttttgtttttgtttttgttttttgctttgttttttttgttttgttttgtttttggtttttgaacagggtttcttgtgtagcttgagctttggagcctatcctggcactggatctggagaccaggctggcctcaactcacagagatccacctgtctctgcctcctgagtgctggattaaaggagtgtgccactaatgcctggctgaggtggtgatgtcttaccactaacaaattatagacctaaaactgaatgagcagatgtctgctgcctcagggaaggaacaccacatccatcttgttttcctctgtggaaagttggtgggttatcaaatgtgctcttcttctcagttttgacaaatagcctttgagaaagccatttaaaggtctgagaccacgaattttctttcttagtttggcctccatttaactgttctcaatgtgatctgtttcagtgaacagtgatgagtttgttttattgaagtacatgaactcagagactttttgaaagaagcagctacccctcatctgcaggtttagatttgagtttgtaaactacttgcaatagcatgtctcctttaaataaaaaatgcatttttgtgttgaaagcaagtttgaaagagtaagaagaaaaaaagcaattttgaaatcagtagtgtacttgcttataaagtaaacattttattttttaggagtaataattatgagagagcagaaaaaaagaaagaaaggaagtacagtacaggccttggccagaatgaaaatagtttaaacctctagtacaagcttgtaaacaaggaactactgcggattggaagtttggtattgtagcaagacagggggaaaggagagagaaggaataccatgttttctctttttgtctgaaataggaaaccttaagcagtagccctttgaaagatgactacaaatcagcttgccatacttgcaaataaagcctgtgactattttacatctctaacctcagcctcccacacagaatgtggaatacagaaggtgcacaatgtatgcttttgtttgtctgttttggtttttcaagacagggtttctctgtgtaacagtcctggctatcctggaacttgctttgtatgccatccttgaactcacagaggtctgcatttctctgcctgccaagttttgagattaaagacatgtgccactaccgcccggcctagatgtttattgaataagtaaatgaacatgcagctattttgtaaatggtaaggaaaaataagcacaacacaggagcacaaggaagctaacacacaatgttattctgtcactggccaacacatgcagaatagtaacacagaatcagtgcaaactaggatagtaccaatgtttatctgtctctaccaaattttatgagatccctaacagatacctaattaatgaaaatggttcttgatactctcatagttaaattatacctgaaacctaaaattctttttttaagatatagttttcttttagaagcactcttatctctttcttgtatcttggctgtgatagcacacctccttagagaatgggctcccaaagttcatttgtgctttagggataaacactggctccactgttacaggtctcatagactgtcctggcctcctagcaggcactgaattcagagagcttggtttagtccaatgctggttccccagctttactactagggtctccatgctctcactaggtcaggtcaactgtttctgttgctttctctaggacagtcttggctcctttgctcatccatcttctctctctgaaactaaattccaggagtatgactcagtgctaagctgtgggtgcctgtttctgctttgatcagctactggatgaaagctataggattgtcaaccaaggtagttatcaatctcattataggggagaggcacaaagacagcctctccaccactgcctagattcttaggtggggtcatccctgtggatcccctaacttttcccctgtgctagacctagttctaaacctatactggttccctctatcaaggcatctcctttcttgcccaccttaattcctcccgtctcagtcctcctattctctcctgttccctccccctcctctcctacctctgtgctcttccacctctgtcctccctaccgtccatgctcccaatttgctcagggtttttgtaccccttcccttcttgaagggaccatatattcttctcttagggtccttgtttcctagtttctctagtggtgtgaattgtaggctgctactcctttgctctatgtctaatatcaacatatgagtgagtagatactcttcttgtctttctgtgatagggttacctaactttggatgttttattccagttccatccatttgcctgcaaatttcaagatttcattgtttttttcccactgagtaatactccattgtgtaaatgtacaatattttctccatccatgctttggttgaggggcatctaggttgcttccaggttctggctattacagaaaatgctgcaagaacatagataacatatgttcttgtgtatgaatgcacatttttgggtataggccaagagaggaattgctggatcttgaggtagactaattcccattttccggagaaactgccatactgatttccaaagtggtcatagatgtttgcactcccaccagcaatggaggaatgttccctttctccacatcatctccagcatagactgtcattggtgtttttcattttagccattctgacagtaataggatggtatctcagagttgttttgagttgcatttccctgatggctaaggatgttgagcaattcttaattctcttttagcca

SEQ ID NO: 21 - DNA, 14800 bp, Cricetulus griseus
  misc_feature: 000562F-005-0125000-45000_Type_C
atgtcatatggttcccatgttcctgtgctcctatgcaatctgattaatatgtcagtcaggttacctgagaacctggtcaatctggattggtaaaaagagcatctaaaaagctcttctttagggaagagtcatgttacacaggcaaacacatttagaccccggctctggggactggagaggtggctcattggttaggagctcttgctgctctcgtggaagacttggctttccttccccactgaggctgacaactgtctgtaactcagatccagggatccaacaccatcttctagcctgcaggcaaatacccacatgtatatacccacacaaatatgcaaaattaagcaatcttaaaaatcaaacaaaacaaaaaactcccactgtctttactaaattaggggtcagggaggagtaaccatgcagtgagtacagtcaggagaatgacactgagccctgagatgaagcttgggactgtgtgggtcccttgctaatatacaatgctgtcctttgaaccccactgctgtgaagcccagacatctgcctgacagatggccagggttcctgctgacatgctgctttggggggaggggactacgtcttagttgtaataatttttgttattttcctctaaatccttgcccttgtccactagtgctctttttttaaaaaattttttacaaacttgtttttgtaaaagcataatcactataggtttttttccccctataaaacattgaccatgtataactagaaagaacactggcgatatttggcatggtcgtcttgatgtggatgccagtgtccacagaagttaaatgacttccccaccagggatggtagatctcagaataaagcagatcactaagatgggtatatgacactgtggttggctcaccattgcccctttggctatcagaagactctcacttcagttttaggaagctctggccctctaccatcagcccacatgactcaaatggaactgttcttctgtctagctactttcttattgctgtggaatggcaccatggccaaggcaacttataaagaaagcatttaattagggccttgcttatagtttcagggagtgggcccatgactatcatagcagaagaaaagcagcaagcagctaggcacagtgttgaagcagaagctgaaagcttatacctaatccgaaagcttggaaggggaggggggaagggaagggagggagggagagggagagagatattgggcctgctatgggcttttgaaatctcaaagcccatccccagtgacacatctcctccaacaaggccacacatcaactggggaccaagctttcaaacataggagtttatgggggccattctcattcaaaccaccacgcacgccttccctccccatagtacttctcaggtgttgctaatcagagccccagtatcagttgtgtggctttttgtttgtttgtttgagacaggatttctctatgtaacagctctggctgtcccggaacttgctctatagaccaagctggcctcaactcacagagacctgcctgcctctgcctcccgagtgctgggattaaaggtgtgcgccagcaccatcccgctggttgtgtggcttctgctgcatctaagagaaactggatttttttttctggagctcttcagctggtagaatagtagcccagaacacccaatgatcattacataaagtctgttatcaaaaaaggcaaccagaagaaaacagaaacaggaagtgaagagtaagcctgagacttgaccacatgacttggactcctggctctaaccagggatgaatgatatcatatcagtctgttttctgttgctataagttaacaaaatacttaaaggctttggaagttggaagtctgtgatcagcctgtaccacccccacctcttacccccacccccatccctgacacccagttatgtcacaatgtggtaaagaagcagaaaagggaagaattcacatgcaaaaacggtggccttactttattgtgatatgctgtcacagtagctaattttactctacaagacctgcattaatctcttctgaggtcaagggcaatcatggcctaaatgaccttgtattaggctcccctcctaaggttccattacctttcaagttgaacatgctggaatcaagcttccagcacacagatctttgaggacaaaccacacccaaaaccataacagatgttattcctctgcagggaacttgtgtgggggggcacaggcatgcatgcattctttgctttgctttaggttgagttgcagtgggtggaagttcctgcaggaatgcatttctaattctctttcctttgcttttctgaaccacaaaaagtgttaatccttggattatgtagcaggatcagctgagtttgggacagtcttgtccagccctttagtcaattccattggctcttcttgcaggatgagtatggcaacgtgtggagaggccatggatgttctagaaccaggagaacacatctgagactgagtcatcctcaatatgtcatgtgaaccagtgcactggacacaggccagggcaaagtcagtcttctcttattacctctttagttgccgccttcctggaaggaaggaacacctgaccctgctctggatttattagttggcctaggtctccaatgtgcccaactcctcctggatactgattatggccagcagcattaagatacgtttaagttggggctggagagatggctcagaggttaagagcactgactgctcttccagaggtctgagttcaattccagaaccacatggtggctcacagccatcattatgagatctggtgccctcttctggtgcagatatatggaagcagaatgttgatacataataaagaaataaaatctaagtgtgtggcgaggctagagagatggctcagtggaaaatcattactgtctttcaaggactgattgttccatgaaagaccctgtcctttagccctttctttctcaagtttgtctcctcttcctcctgtcggcggcttccccgatcccaccccggtggcctttcccgcccggccgagaacaagcaccggtgggccggccgagaaagcaccggtgggccgccgagaacgagcaccggtgggctggcacgagggcgagcaccaggtgggtcagcacgagaacaaacaccgagtgagccggcctggagcctgcccctgagccccgcccgcccgaagagaaacactccgtcccaaggtctccgccccaaggtcagccatcaggaaggggggggaattgagtctgctgtaccagacaccagaccttgagaatatgctgatctggaatggctctgtgtctcatttgaaccatccaatagaaataattctgtatttcgcctcatttgaaagactctgtgtttcacctcatttgaataactctgtactttgcctcatttgaataaccctgtatagcgcctcatatacattgaccaatgggaatagctctgtataatgcctcattagaattatccaatagaatccctgctcctagcttgcgcctttttcctatataaggacccctttcccttggctcggggcgcttagccacacagaagctaagtcgcccgggtacccgtatctccaataaagcctcttgttttttgcatccagttcgtggcctcgctgattcctgggtgtgggtctcctctacgaaagtatcccttcggggtctttcatttgggggctcgtccgggatcgagacccgcccagggaccaccgacccacgtctgggaggtaagtgttgtgcggatccgctgttttgtcttgtctggtctgagtctgtcttgtgaattgcgcttgcgtttgtagtatacagctgtgtacatttgtaggcggatccgaggaggactgacggtcgaactccgaccgcggctccaggagacgtcctggtagcgtttgaagccctcaggagagggatttgtatttgaacttggaagccctcaggagaggatttgtatttgaacttggaagcctcaggaggattgtatttgaacttggaagccctcagggtgagagatttgtactttgaacttagatctatgactggacattttcccagtctctttggagaaggccctcggcttgagggatttgcaatctttactgggacgaggaaggagggccccttcctcgactctctctcaattccttctgtcgactctctgtcgaaaccgcgctgcgaaagtctgttctgtgttattcggtctttgtcttgtagctgtcatttgtgcctcctaagcctagaaactatggggcaaactgtcaccactcctttgtccctaacactctccactggaaagatgtacaggaatatgctcataaccaatctgttaatgtgcgtaaacgcaaatggattactctttgttcttcagaatggccgacctttgacgtaggctggccgcgagatggtacctttaaccccagactatattccagataaaagagaagattatggatcctggaccacacgggcatcccgatcaagtggcttatatcgtcacttgggaggctttggttcaggaccccctccctgggtacgtcctttcttacatcccaagggcccctctctcctcccccctctaaccgctccaaccgacccattccttcggcccctacacctcccactcctttgattcctcccaacccccttcccattccaacctttaccctaccgtggtgaaagacactaaggctaaagaaaagaagacacctaaggtactcccctgggagaagaccagttggttgatctattaacggaggagcccccgccatatccgccactgccgcccccaccagaggcagaagcggactccgccgcgccttggcggaagcggcccctgacccttcaccaatggcttatcgactaagaggtcgtagggagcagcccgttccagattcaaccactctgcccctccgaactggctgaacggccaacctcagtattggccattctcagcatcggacctctataactggaaaaataataatccttctttttctgcagaccccgtgaggctgacatctctcatagagtcggtactcacgactcaccaacccacctgggatgattgtcagcagcttttgcaggtcctttaacctcggagagaaacagcgcgtgctactagaagcacgaaaaaatgtcccaggagtaaacgggcagcccacccagctgcccaatgaaattgatgcggcttgccctcttgaaagacctgaatgggattttaccaccgaagcaggtaggacccacctgcgtctctatcgccagttgctggtagcgggactccggggggcaggacgcagacccactaatttggcccaggtgaagcagataatacagggtgcggaggaatcacctgccgcttttctagagagattgaaagaagcgtacaggatgtatactccctataatccggaagatccaggtcaggccaccaacgtttctatgtcctttatttggcaatcagccccggacataagaaacaagcttcaaaggctagaaaatctacagggatatacactccaagatttgttgaaggaagcagaacgtatttttaataagagggaaacacagatagaaagagaagaacgttggaggaaggaaactcaggagagagaggaaagactaagacaggaagctgaggaaaaagaggttgcgagagaccgtaagcggaataaagaaatgagcaggttattggccacagtagtgacaggccagagacagaatagacagagggatgacagaagggggccccacctggacagggaccaatgtgcttactgtaaagaaaaaggacattgggcaagagaatgccctaagaaccccgggccaagcttccaccgccaagggtttctgacctcctgaacctagaagattagaggagtcgggccaggagccccccctgagcccaggataacactgcaagtcgggggcatccggtcaccttcctagtcgatacaggggcacaacattccgttctgaatcggtcacccggacccctgagtcaccggactgcatgggtacagggagctacaggcggaaagcagtaccattggactacaaatcggcagctccagctcgcgaccggtaaggttatgcattctttcctccatgtgccagattgcccctaccccttactaggacgggacctattgaccaaattaaaagctcaaatacactttgagaggtcagaagtcaaagtcacagggccagagggaattccccttaccatcttgacaatgtccatagaagatgaatatagactccatgaaaagaggactaattcgaacaatcaggaaacccttgatcactggcttgcggaatttccccaagcctgggctgaaacaggaggaatgggccttgccattaaccaggccccaattatagtaaccttaaaagctgccatccttcctgcatccgtcagacagtatccaatgcctaaagaagcccgcgaaggaattcggccacatattaaaaggttacttgaacaagggattctggtgccctgtaaatctccttggaatacacccttgctacccgttaggaagccgggaactaatgactaccggccagtgcaggacctgagagaagtcaataaaaggatagaggacatacaccctactgtccccaacccttacaatttgctgagtggattgccacctaactatacctggtacacagtcttagatcttaaagacgctttcttctgcctccgcctgcatcccaccagccagcctatatttgcctttgaatggcaggatgcggcccttggaatctctgggcagctgacttggactaggctacctcaagggtttaagaacagccctaccctttttgatgaagctttacatcaggacctggcagaattccgggttaggtaccccgctctaatcctcttacaatatgtagatgacattctcctggcagccaaaaccaaaggggaatgcaaggaaggcactcaagccctcctccagactcttgggagcctagggtaccgggcatccgccaagaaggcccagatatgtcagaaacaggtgacctatttaggatacaagataaaggatggacgtcgatggctaacggaagcccgtatgcgagccatcttagacattcccaccccacaaaatccccgccaactgagagaattcttgggaacggcaggcttctgccgcctatggatccctgggtttgccgaaatggcggctcccctctacccctcactcggccaggggttgctttaaatgggaagagccccaaaagaaagccttcaccgacatcaaaaaggctctccttgaatcaccagccctgggtctaccggacttagctaagccatttgaactttttatagatgagaagagggctatgctaagggagtcctcacccaaaaactggggccttggagaaggcccactgcatacctctccaagaaattggatcctgtggcatcgggatggccaccctgccttcgaatgattgctgctatagccctgctggtaaaagattctcacaagctaaccttggggcagcctttgaccatacatgcccctcatgcagtagaggcagtcatcagacagcctccagacagatggcttactaatgcccgaatgactcattaccagactatgctgttagacaaagaccgggtccacttcgggcctttggtgactctgaacccagccaccctgctccccctccctggggagcccgaggctcatgattgcttacaggtattggccgaggcccatggagcgagatccgacctgactgaccagcctctacctagcccggaccacatctggttcacggatggaagcagctttttgcatcaaggagaacgaaaggcgggcgcggcagtcaccacagagaatcaggtcgtctgggcccaggcactccccctggaacttccgcacagagggcagaactcatagcactcacgcaggctctaaaattggcagaaggtaagaggctcaccgtgtatacagacagtcgttatgccttcgccactgcccatatacatggagaaatttacagacggaggggctgcttacctccgaagggaaagacattaaaaataaggaggaaatcctcgctctcttaagggctcttcatctgcccgctgccttaagtatcatacattgccctggacaccaaaaaggggattctttcgaagcaaggggcaatcgaagggcagacttggctgcccgagaggcggccctgaccacagacaccactaacctcctggctctagagcccaccaacgaccatcccttcccctcatgggactatgaacaaagagacatccaaaccctagagaaattgggagccgcaaaggaaccaaacggggattggacttatgaaggaaagactgtcatcccctaccgggtaaccaagtacctagtgacatttttacataagatgacacatctgagctccaagaagatgcgggagctcctcgaacgagaagaggaattcaatttccttttggggaagaacgatattctaaaacaggtaactgagcaatgtgatgcgtgcgcccgagtcaacgcatccagactgaagcttcctcccgggaaccgggtcagaggctaccggcccggaacacattgggagatagatttcactgagattaaaccaggaaaatatggatacaagtatctattaatttttgtagacaccttttcaggatgggttgaagccttccctactaaacatgaaacagccaagatcgttactaagaaattgcttgaagaaatctttccccgttatgggatgcctcaggtatgggaacagacaatgggcccgccttcgtctcccaggtaagtcagtcagtggccaccttgttggggattgattggaaattacattgtgcttatagaccccaaagttcaggacaggtagaaaggatgaatagaacaatcaaggagactttaacaaaattgtcgcttgcaactggcactagagactgggtcctcctactcccctagcactctaccgcgctcgtaatacccctggaccacatgggctcacaccctttgagatcctgtatggagtacctactcctatcattaactttcttgatcaagatgtctcagattttgctaactccccttctctccaagctcatttacaggccctccaactagtacaacgggaggtctggaaaccccttgctcaagcttataaagaccagagggaccatcccaccatccccattcctaccagatcggggacactgtttgggtccggcgtcaccaggccaagaaccttgaaccccgctggaagggaccctacatcgttttgcttaccactcccaccgcactcaaggtagacggcattgcagcttggatacatgcttcacatgtaaagccagcccgacccaccgattcagccactgcatcagaatggaccgcacaccgcactcaaaatcctttaaagataagactctctcgtacaccctcctgttgattggttgtctgtttacccccatgtagcaactaaccccacagggtttataatatcacctggaaaatagccaatctagggaccggggaaatagccaacctcagcacttatatagggactctacatgatgggttccctcctctctatgtcgacctatgtgacttagtagggtctgattgggatccctctgaccaggaaccattcccagggtacggatgccaccaccctgggggaaggataggaacaagaagcaaggatttttatgtttgccccggccataaaccaactcatggctgcgggggccgcaggaagggtactgtgcaagatggggatgtgaaaccacaggggaggcttactggaaaccctcttcctcttgggatttcatcactctcaaacggagggagatcccagggtacgcagggaaaggaccatggagatgtgggcaaagagcctgcggaccttgttatgatagtgccggagggggaggttttcaaggcgccaccccggaggaaaatgcaaccctctcatcctaaggttcacagatgctggaaaaagaactacttgggatagtcctaaggtctggggactcaggctgcaccgagcagggaaagatccggtgactttattctccctgtacagacaaattactccctaagccaacaatcagtcgggccaaacatagtaatagcggaccagagatccccaacccattttcaagtccctaaacccctaccgttcctaaagctatcactcctacaccaggtgctgcaccttctccccaccccagatgccctaaacatcgagataaccagagaccctccaggtaccagagatagattattacaattaatccaaggagtttaccaagccttaaatttttcagaccccaacaagactcaggaatgctggttatgcctagtttcccggcccccatattatgaaggcgtggcaatactgggcaactactccaaccagacctcagcacctaccagttgcggagctgctatgcagcacaagctcacaatatctgaggtctcaggaaaggggctatgcataggcaggattccttcctcacatcaagaattatgtaaccaagtagagccattatctcaggacagccgataccttgttgccccttatggaacttattgggcttgcagtactgggttgactccctgtgtctctaccactgttctcaacaccaccattgacttttgtatattgatagaactttggcccaaagtcacataccaccaacctgaatatgtttacagcgtactagagaaatcaacccgatataagagggagccaatatcctttaccgtggccctattattaggaggaataacatgggggcatagcagccggcatagggaccggaaccgttgccctacagggaattaatcattttaagcttctacaacaagccatgcacacggatatccaggtcctagaagagtcagtcagtgcactcgagaaatccttaacatcactctctgaggtggtcctgcaaaacagacgaggattagatttattatttttacaggaagggggctatgtgctgccctcaaggaagaatgctgcttttatgcagatcatacaggaatagttagggatagcatggccaaacttagggagaggctaaaacagaggcaacagctatttgagtctcaacaaggatggttcgaggatggttcgctaatccccctggttgactacccttatatccacgctcatgggacctctggttattctatttttgatcctcatatttggtccctgcattctgaacaaactgactcaattcatcagagaacgactatctgttgtacaggccttagtcttaactcaacaatatcatcagctaaagcaaatagatccagagtatcagagacctctgaatgaaagattccattcagttacaagagaaatgggggaatgaaagaccctgtccttagccctttctttctcaagtttgtctcctcttcctcctgtcggcggcttcccgatccccaccccggtggcctttcccgcccgcccgagaacaagcaccggtgggccggccgagaacagcaccggtgggccgcccgagaacgagcaccggtgggcggcacgagggcgagcaccaggtgggtcagcacgagaacaaacaccgagtgagccggcctggagcctgcccctgagcccccgcccgaagagaaacactccgtcccaaggtctccgccccaaggtcagccatcaggaaggggggggaattgagtctgctgtaccagacaccagaccttgagaatatgctgatctggaatggctctgtgtctcatttgaaccatccaatagaaatgattctgtatttcgcctcatttgaaagactctgtgtttcacctcatttgaataactctgtactttgcctcatttgaataaccctgtatagcgcctcatatacattgaccaatgggaatagctctgtataatgcctcattagaattatccaatagaatccctgctcctagcttgcgcctttttcctatataaggacccctttcccttggctcggggcgcttagccacacagaagctaagtcgcccgggtacccgtatctccaataaagcctcttgtttttgcatccagttcgtggctcgctgattcctgggtgtgggtctcctctacgaaagtacctcttcggggtctttcacccacatgggtggacacaacctataactccatctccagaagacctagtgtcctttttttctggtctctaggagcaccctgccaatatacatacacataaataaaaaataacaacaaatcttttgtaaaagttatctttaaacggctctcattactctatgtcctctactttctcactttctattctgttggagttcttttaggttacagatctatctggctggatcaaagagctctgtcttgtggaactgtataaacaatcacgtgaaaacaagcagggctgacctgacagaaattgctatagagagtgtaagcctggcaagatcagacacctcaaaacttcctctctccacttctttactcctccctctctctcctctccttttcttgccatctttagtattctgccctctctctactccctctccaccctccctaatgggtcctccagagagggtggaaacaagttggctcagtcattttctttggataggaagacaagtcatagatcactggcctcaagttcaggctagggctatttagtgcttcagggtcaggaagtaggggtagcatctggcttagtagggctgtggaagggagaaggagagagccataaggagcatctttgtatgcctcttcactcttacatccaaagcaactggtccgtgtgctcagcaacccggaagctgctctcagtgtgcgcataagcagcgcctttgttactggaccaaaatgggtggtattcattgctttctgtgcaccagtgtcctctggttctccacattgctgactctttccttgtaatctccttgcctcctttaccctcctactcctttgctgtttctttgctttttctgtggacttttcttcctctttctctacttaaaaggcaggcttcctctgcatgggttttacactcttctccaggttctgtgcatttttgcctttgtttttgttttttccctacttcctccctgtcaatcacattctccacgtggcctcagtcagttctgctctgtaccaacagattccaacactatacatctatctgctcagcctcctggactgcttaatggaccagactgggcagacagttttgccaatatatcccgcaggtctctacacgctcgggacaactcagcaccctctgcctcacagtgtctcttcccctcatgggcctgacttcaactggtggtgccacccacctgttcaaactgagaatagcatcaactcaaggtctacctctccctcactctacagcaagcccgataaccacacataaaagagtcagtgatgggcttccatatgctaaacaccccctgtctctaagcatctcttttcttttgccctctcaccactcctggggtgcttcagaccctgacaagggtcttcagttttccagctccccagtttagttaccaaacagtgctgatggctcttttccttcctcctttcagttttttaagatgtatttttattgttttaactgtggtgggcgttggcttgtgcatgtgtgtttgggtgactgaggggatgccagagttatgctccctctgaagctgaagtaccaaggcagttgtgagccttccaatacgggtgctgggagctgatctcagagcctccgcaagagctgtgcattcccctaatatgcatggagcggcctctccagctccctactccttttaaaaaacaaacatagtggggctggagagatggttcagaggttgagagcaccgactgctcttccagaggtcctgagttcaattcacagcaaccacatggtggctcacaaccatcgttatgagatctggtgccctcttctggtgtgcagatatacatggaagcagaatgttgtatacataataaataaataaaatcttaaaaaaacatagtattttaaatgtaaaaaaaaagacgcaaatcactacaaaatggaagcaggcttgctttaagataagtgtatgtgggagctggagagatggttaagggcactgctcttccagaggacccaattcaaatcccagcacccatatggcagctaacaactgtctataactccaagatctgactctcacgaagatgtgcctgaaggcaaaacaccaacacacaggaaataaagtaaataaattaaaaaaaataagtacgtgggggctactcttaagcttgactattcagatttttgaacatatgctaaagaaaacagaatactggatgtggttcagtggtagatcatttacctaacaggtacaaggccctgggttcacgctctggcatggcataaaacaataaagaaggcaaaaatgtcatatattatagattatatcatcattattctctttcatcccccataagcatcaatgatgtgggccagtactgccctgttccctacctctcacatagtattttgaaataaatgcaatatattccatcattttactcacaaatatttcaatatagattcctaaaaggtaaggatttgcctaaaagaacaaacaataattccttaatatcattaagtactgacattcagatttccagttgactacatagagacaatgttccctttgtataggaatatttcaaataagaatattaaaaagccaagtcaggcag

SEQ ID NO: 22 - DNA, 4677 bp, Cricetulus griseus
  misc_feature: 001329F-001-011-4677_Type_C
ggatccacacgatgctgtctgacttgggattccatttcttcctgtaggggattggccagcctgtccacacttcagggcttcatataaaagaccttcattgtctcctcaatgaagaagacagaatcaggttctgcaggactaggactgagcaaagggagattggacactgccttggatcttcctcatggagagaatggattctgagtctggacattcaatggctgcctggtggatgtgaactgacacataactgtgtctggtatgtggaatccccacaagcagaggccatgagaaagccagcgcctttgcagctggaagatttagcattcatatccttgcttcccttccatgaggattctctagcttcttccctgctgcttctgttctatggatctcttcctcttctcgtttgtttcacaagcctgatcaatttgggagaaaatggatgcctggtctactgtattgttcaagcagtcgagttgttgtgaaggatcccaagttatggtcgctgacacatggaaaaaaaggattccaagtgggagacaaatttcaaatggctgaatacagtgggagcactgacacctgatggtctctcacacataggatctgagttggctgaccttgcaagaggacacaatctttgggaagtccacacccatcatgtgatgactggtggcgagcttctcagcttgttctctactcttctgcatctactgccctccatagcttaactctttcagaagcttttcttctcacacttgtgatccacaatggagaagtcgggtcacatgttaagagttaattcagggactaagttgaggagtactgcagattcttgaatggccctggcactccgaatcgggactgagtgatatgccaagagggacagaccctgttgggaggcttgtctcctgagatccacaggcaggattcacatggacttctcaggccttttctgattctctttagcatgtctggtcttgaaccagttgtaaagtagcaaacaaaagagatcaggagaggcagggtgccccaaaaggccccaaatcttaagatgctggaagaaatgcctggcctatgctctttatacttcatctatcacagatttcaaagatgaagggacatgtatagaatgaaggcaggatgaacaggacttgaagccaggagaatatggaaggttctaatacaaggatgacattttaaaggaagccctaggtagaggctattaagatcctttggagcaaaattatcgctctgaggggcaccaccattctgtatttgggactgagagttagaccaacctaagataacacttttatctctattccatgagatttattgggctttcagccgcagtacttgagggagaaatgtgtgcacaaatcagggaaatatgcatatgcctgctgggtcaggggctatctgataagagtgaagggagacactagacaggccatacctcgatatggtgctcctccaggccaaacctttgaaccagagtcattcctccttcaggtacatgcacttatcagaatgttcttgcagtacaggtttttatcctcagtgaatgtctgcatttctgctgcctccttggattattggaagactcttgtgactttggaggaacttgagatgtcaggagaccatgggatatggtgatgctgaggtacttgtagaaccttgggtgactgggttcagtggagagctagagatctgttactttgaagtgtatcggtcctccagtcctttttcaccaggtggaattgacaagccatgggaacatgaaattctgggatcatatgggaatttggtccattgtgagatctttgtagactaaagacactgggaataaaaacagaaatagatcagagaagccctaaagagcctagctcatccacacatagctccctattgcctgttgcaggcttggtagataatgtcctgctcaggtttcatctatggtggtgaacactgtagaaaagcatattagatgggacagtacccttgggtctcatcagcactgtccttctggttgccccaccttttgcctgaaaggggtcaattaaactaaagccaggagaatatcaaaggttctaatacactctcgcaatttctgcaaaattgttgcataggtatacacaaagagttaaccatgaaaggaaacaaaaacgacacaaatatggtcactgaaaaatgttagtactcttgtgtccaaattctaagccacaagttataaaaaccaacatgtgggaagtaaagcaagcttctcccagagcattaaaggtcatcctgggctacagagaatcagaactttgtctcagccaagaaaccaaactgcattgggtagaactgctctgatccaaagtgtttgtttacttgctcaactatcagtatcagcagttggtaaaagaagtcaagtaatgagaattgcttaaagaattcaagttgggacacatttcctagagaggcatctaattcacagagttgttcaataaacagctggtcctggatggcagaagaggctccatcattatctttggctggaatggtgagagctgattgagtctcccatcgtcttccagcaggcttgtgtcaggctctggaactgttgatgaagctgtgatgaattcaccagtctttgttgaatcagaatcttgatctggacctgggtatgcaggtgatccacaagatgctgtctgacttgggatccacaagatgctgtctgacttgggattccatttcttcctgtaggggattggccagcctgtccacacttcagggcttcatataaaagaccttcattgtctcctcaatgaagaagacagaatcaggttctgcaggactaggactgagcaaagggagattggacactgccttggatcttcctcatggagagaatggattctgagtctggacattcaatggctgcctggtggatgtgaactgacacataactgtgtctggtatgtggaatccccacaagcagaggccatgagaaagccagcgcctttgcagctggaagatttagcattcatatccttgcttcccttccatgagagtctctagacttcttccctgctgcttctgttctatggatctcttctgctcttgttgactgcctctaattctgatcaatctgggagaaaatggatgcatggtctagtgtattgaagcgattgagcaggaggagtcacaagtgttggtccctgacatatgggtatctgctgcccctaccaggctgcaggtgaatctacaatcgatgaagggtactagagatttggctgggactggattttatctctgcagctgagtctgctttgtcctgaaactactccttagactaggagtgtcttttctagatggaaatgatccagaagttactgtacctggggcttatccaaccagtgctgaaagacccctgcccttagccctttctttctcaagtttgtctcctcctcctcctgtcggcggcttccccaatcccaccccggcggcctccacttccccgccacccgccgcatgcccggcctgagaacgagcaccaggtgggccggcacgagaacgaacaccaagtgagccggctggagccctgccctgagcccctgcccgatgagaaacactccgtcccaaggtctctgccccaaggtcagccatctggacgcggaggggggggaattgagtctgttgtaccagacaccagacacctagggtactcccccgggagaagacctgttggttgatctattaactgaggagcccccgccatatccgccaccgccgcccccaccagaggcggaagcggcccccggacccttcaccaatggctcatcgactaagaggtcgcagggagcagcccgttccagattcaaccactttgcccctccgaactggactgaacggccaacctcagtattggccattctcagcatcggacctctataactggaaaaataataatccttctttttctgcagacccgtgaggctgacatctctcatagagtcggtcctcacgactcaccaacccacctgggatgattgtcagcagctgttgcaggtcctcttaacctcggaggagaaacagcgcgtgctactagaagcacgaaagaatgtcccaggagccaacgggcagcccaccagctgcccaatgaaattgatgcggcttgccctcttgaaagacctgaatgggattttaccaccgaagcaggtaggacccactgcgtctctatcgccagttgcttgtagcgggtctgggggggcaggacgccgacccactaatttggcccaggtgaagcagataatacagggtgggaggaatcacctgctgcttttctagaaagattaaaagaagcgtacaggatgtatactccctataatccggaagatccaggtcaggccaccaacgtttctatgtcctttatttggcaatcagccccggacataagaaacaagcttcaaaggctagaaaatttacagggatatacactccaagatttgttgaaggaagcagaacgtatttttaataagagggaaacacagacagaaagagaagaacgctggaggaaggaaacccaggagagagaggaaagactaagacaggaagcagaggaaaaagaggttgcgagagaccgtaagtggaataaagaaatgagcaggctattggccacagtagtgacag

SEQ ID NO: 23 - DNA, 13171 bp, Cricetulus griseus
  misc_feature: 000386F294053-310687_Type_C
tggtccccgacactaaccaagcaatgcacatgtggagttatgcctgcaggagctgctgacatggggtattggtgaacctgcactgggtggctcccagtttaaaccctgctaggataaagggctggtgcttagcgaatcccagcccctgtctccacaaaagcctctgaaaagtcagtgaacctcagacctaattctcggggttgtgtgggtgtctctgacagtctgtattcatcttctagccatatagttaaaagctgtataggtcttactcctgagtcagttacagttaccaagtcactgtggaacgatatccgagcttccacctttgtaagtgaatctcttcccccaaaaggggtgtatggacagtcaggatgaccaggagggagtggatacctggccactccctaagttcactcaccttcaggcagcccatgaatgagtagtggcccatgccaatagcccctgtacccaagtctttcactttgagattagactcagaggggtagtcagaactgaatgctgagcacctgtatcaaccataaactggacaggctccccctcccaatgcagggttaacttaggtttggggagagggtcctgaccccatccccttcagtcactgtcttcctctgcttcagtcaccagtacatgaggggactctttgtggtctctttttctgggacatttcctggcccagtgtccaacctccccacattaggggccctgatgtttttaaaactgcatcttgttctcttgggaggggcggggagctacctccttcccaggcccggtggtggagtcgtttttgcttctcctctggctccacagccagtagcacctggaccatattgtgagcttgcctttcatctgcagccactctagctctccgatccttttccctatctactgtccaagctctctcctgtctctcacttccctcaatcctcactataacttgtctttcccggaagtctccctattactgtataccttgtttgctaccttcatgagctcgacttgccagctaagccatctatcttctggagcttcgtcccagtatcgggagctgcctggctgcctcagggtccttggggagtatgccctaaagacatccactaatctctcaagagaggctgatggtgattcaccagcttcctgtcagacatcatacattttagccatattagttgggtgctgagcagccgcctggaaacccctctttagaccttgaggcgctcccgaccttctggataactgaaattccattggggtcgggtcaaggggaagtccacatggatcagtggctgtcgcctggaaccactttctgggcctcaacaataagctccttttctttggtagtggagcggatctgaaggagcagttgccccaggcaggctggagagtaaatataacagagtccaataggttaataagaccccccgggctctcagaaaaagggggagtctggtttttcctcattgccttgaccatccagcaggcctatcacatgaaagggaagggcagtggcatctgcctcacaggcagaagcttcctggaagcattcattcaatcttccctggtcattgtgagagagacttgggatgggttcagcctccccaggggcatccatggagcagagggaaggtatgcgtggggacagatgttcttcctctggtaccccttggagaatggggggggcgtgtcaggatctttggggatgtccttggtgttccccaagacaaatacttttgaagaccagatggtggtaagtagggttttcaaccaagatgggtatgtgtgtgtgtccatgatcaggttctcctagatgctaaagtagggaacctggtccatagatgtcatctttagccctactaatgataggttaaaagtgccctctcagcagggcgttggtggcgaacgcctttaatcccagcactcgggaggtagaggcaggtggatctctgtgagttcgagaccagcctggtctacaagagctagttccaagatagcttccataagccacagagaaaccctgtctcaactcccccaaaacaaaaacaacaaaacaaaaaacaacaacaacaacaaaaaaaacaaaccaacaacaacaaaacaaaaataaaagtgccctctcctggggtgtggagagctggctcagaggttaagagctctttcagagttcttgagttcaattcccagcaaccacatggtggttcacaaccatctgtaatgagatctggtgccctcttctggcctgcaggcatatacacaaggagaacagtgtatacataataaataaattctttttttttttggatacagcgtttctctgtgtagctttggaacctgttttggaacttgctctggagaccaggctagccttgaactcagagatctgcctgcctctgcctcccaagtgctgggtctaaaggcatgtgtcattatcacctggcctaataaataaatcttaaaaaaaaaaaagtgcccttgaaagaccccagaagaggtactttcacagaaggagacccgtacccaggaatcagcgagaccacgaacttggatgcaaacagcaagaggctttattggatacacgggtacccggggcgacttagcttctgtgaggccaagcgcaccgagccaagggagaggggtccttatatagcaaaaagcatagtacagaagcgaaaagcatagttacagggattctattggacaattcaaatgaggcacagagttatttcaggttagcaagttctcaaggtctggtaatcagacgaagctgacttggctctatcttgggtacacagtggttacaacagacccggtagttcctccctccgcgtccagatggctgaccacagatatcctgtgtggcaaacaagtagtcataacgccctgaccttctattctcagaaacggttctgggtggtcttggtctcttggtctgtaatttagctagcccaagagagagagagagagagcttaaggggcagtggtctttcattcccccatttctcttgtaactgaatggaatctttcattcagaggtctctggatattctggatctatttgctttagctgatggtattgttgagttaagactaaggcctgtatgacagatagtcgttctctgatgaattgagtcatcttatttagaatgcaggtaccaaatataaggattaaaaatagaataatcagaggtcccatgagaatggatataagggtagtgaaccagggggacttagcgaaccatccttcgaaccatccctgttgagactcaaatagttgttgcctctgcttaagtctttctctaagtttggccatgctgtccctaactattcctgtgtggtctgcataaaagcagcattcttccttgagggcagcacatagtccccgttcctgtagaaataataaatctaactctcgtctgttctgcaggactacctcagagagtgatgttaaggatttctcgagtgcgctgactgactcttctagggcctggatatctgtatgcatggcttgttgtagaagtttgaaataactagtttcctgcaaggcaaccgtactggtccctcttccagctgctataccccccattgttattcctcctaataatagggccacggttaaggacattggctccctctgatgtcgggttgatttctctagtacgctgtaaacatactcaggttggtggtatgtgactttgggccaaagttctgtcaatatacaaaagtcaatggtggcattgaggacagtggtagagacgcagggtgtcaaaccagtactgcaagcccaataagttccataaggggcaacaaggtatcggctgtcttgagataatggctctgtttggttacataactcttgatgtgagggaggaaccctgcctatgcatagcccctttcctgagacctcagacattgtgagcttgtgctgcatggcagctccacaactggtaggcgctgaggtctggttagagtagttgcccagtactgctatgccttcataatatgggggccgggaaactaggcatagccagcattcctgagtcttgttggggtctgaggaattgagggccaagtaagccccttgtattagttcaagtaatctgtcccttgtccctggatatggggaggtgggggtgtgaactggagtcgtaagagtaagggtggcagtattggatgagggtgttgatgagggtgggagatgggatagaggcctatggggccgagacccatgaggcttggtgggggtttctggtctgccagaactgggtttgggcccatggttttggtgatagtttttactttgagtctaattttaaagatggccccccggtctggtcccgacaggtaccagcgaaggccccaagcatatccgttctgccattctgttgctttcttaccctcatccgtaaaggaaataaggagaggcatagaggtggtgccggtggagcccttttgccatcctctggttacttgaatcttgtcccatgaggatgaaggcttccagtaagcatcccctgtcgtttcacatccccaagtggcacagaaaaactgttcataacccccacaagtctgagtcttcttccaggcccgtccgtctctgggacagacataaaagtcactacccgacagttggtctttggcctgttgactgctacatccaggagtggtgtctctcgaactttggagtgacctgcgaaatcgtggcaaatcgttttcgttgctacaagccttagggtaacagtgtgagatgtcccaggtgtctagaccagcgaccaagtcacagaaatcgggtgtcaatgtaggccaccatacaccaagggcatggtttttggtggtctcataaatgacctcccctgactgggaaagtacctgccaggttaacgcatgaatggtataggggctggttgctacatgggggataaacagacaaacaatcaataggagggtgtacgagagagtcttatctttaaaggattttgagtgcagtgcgcagtccattccgatgcagtggctgaatcggtgggtcgggctggctttacatgagaagcatgtatccaagctgcaatgctgtctactttgagtgcggtgggggtggtaagcaaaacgatgtagggtcccttccagcggggttcgaggttcttggactggtgacgccggacccagacggtgtccccgatctggtaggaatggggaatggtaggatggtccctctggtctttataagcttgagcaaggggtttccagacctcccgttgtacaagttggagggcctgtaaatgagcttggagagaaggggagttagcaaaatctgagacatcttgatcaaggaagttaatgataggggtagatactccatgcaggatctcaaaaggtgtgagcccatgtggcccaggggtattgcgagcgcggtagagtgctagggggagtaggaggacccagtctctagtgccagttgcaagcgacaattttgttaaagtctccttaattgttctattcatcctttctacctgtcctgaactttggggtctataagcacaatgtaatttccaatcaatccccaacaaggtggccactgactgacttacctgggagacgaaggcgggcccattgtctgtccccagtacctgaggcatcccataacggggaaagatttcttcaagcaatttcttagtaacgatcttggctgtttcatgtttagtagggaaggcttcaacccatcctgaaaaggtgtctataaaaactaatagatacttgtatccatatttacctggtttaatctcagtgaaatctatctcccaatgtgttccgggccggtagcctctgacccggttcccgggaggaagcttcagtctggatgcgttgactcgggcgcacgcatcacattgttcagttacctgttttagaatgttattcttccccaaaaggaaattgaattcgtcttctcgctcgaggagttctcgcatcttcttggagctcagatgtgtcatcttatgtaaaaatgtcactaggtacttggttacccgttaggggatgatagtctttccttcataagtccaatccccgtttgattccttggtggctcccaatttctctagggtctggatgtctctttgttcatagtcccatgagggggagggacggtcgttggtgggttctagagccaggaggttagtggtttctgtggccagggccgcctcttgggcggccaaatctgcccttcgattgcccctcgcttcaagagaatcccctttttgatgtccggggcaatgtatgatacttaaggcggagggcagatgaagagctcttaggagagcgaggatttcctccttattcttaatgtccttcccttctgaggtaagctaccccctccgtctgtaaatttctccatgtatgtgggcagtggcgaaggcataacgactgtctgtgtacacggtgagcctcttaccttctgccaactttagagcttgcgtgagtgcaatgagctccgccctctgcgcggaggttccgggagggagtgcctgggcccagacgacctgatcctctgaggtgaccgctgcaccagccctccgttctccttgatgcaaaaagctgcttccatccgtgaaccagatgtggtccgggctgggtagaggctggtcagttaggtcgggtcttgtcccatgggcctcagccagtacctgcaagcaatcatgagcctcgggctccccagggaggggggcaaggtggctgggttcagagtcaccaaagggccaaagtggacccggtctttgtccaacagcatagtctggtaatgagtcattcgggcattagtgagccatctgtctggaggctgtctgatgactgcctcaaccgcatgaggggcatgtatggtcaaaggctgccccaaggttagtttgtgagagtcttttactaacaaggctatggcggcaatcatttggaggcagggtggccatcccgatgccacaggatccaatttcttggagaggtatgcagtgggccttctccaaggccccagtttttgggtgaggactcccttggcatagccctctttctcatctacaaagaactcaaatggcttagccaagtccggtagtcccagggctggtgattcaaggagagcctttttgatgttggtgaaggctttcttttggggctcttcccatttaaaagcaatccctggacgagtgagggggtagaggggagccgccatttcagcaaacccggggatccataggcggcagaaacctgccgttcccaagaactctcttagttggtggggattttgcggggtgggaaggtctaagatggctcacatacgggcttccgttagccatcgcagtccgtcctttatcttgtatcctaaataggtcacctgcttctgacatatctgggacttcttggcggatgctcggtaccctaggctcccaagagtctggaggagggcttgagtgccctccttacattcccctgggttttggctgccaggaggatgtcatctacatattgtaagaggattagagccgggtaccgaacctggaatcctgccaggtcctgatgtaaagcttcatcaaaaagggtagggctgttcttaaacccttgaggtaacctagtccaagttagctgcccagagattccaaggtcagcgtcctgccattcaaaggcaaatataggctggctggtgggatgcaggtggaggcagaagaaggcatctttaagatctaagactgtataccaggtatagtcaggtggcaatccgctcagcaaattgtaagggttagggacagtagggtgtatgtcctctatccttttattgacttctctcagatcctgcactggtctatagtcattggttcctggcttcctaacgggcaacaagggtgtattccaaggagatttacagggcgtcagaatctcttgttcaagtaaccttttaatatgtttccttcgcgggcctctttaggcatcgggtactgcttgacggatgcaggaagggcggcagcttttaaggttactataattggggcctggttgctggcaaggcccattccgcctgtctcagcccaggtttggggaaattccgcgagccagtggtcaagggtttcctggttgcccgagttagtctctttttcatggagtctatattcatcttctatggacattgtcaagatggtaaggggaattccttctggccctgtgactttgacttttgatttctcaaagtgtatttgagcttttagtttggtcaataggtcccgtcctagtaaagggtaggggcaatcaggcacatggaggaaagagtgcatgaccttaccggtcgcgagctggagctgccgatctgtggtccaacggtactgctttccgcctgtagccccctgtacccatgcagtccggtgactcaggggtccgggtgactgattcagaacggaatgttgtgcccctgtatcgactaggaaggtgaccggatgccccccgacttgcagtgttatcctgggctcaggggggggggctcctggccctgacttctctaatcttctaggtttagaaggtcagaggttcttggccgtggaggcttggcccggggttcttagggcaatctcttgcccaatgtcctttttccttgcagtaagcacattgatccctgtccaggtggggcccccttctgccatccctctgcctattctgtctctgacctgtcactactgtggccaatagcctgctcatctctctattccgcttacggtctcttgcagcctctttttcctcagcttcttttctcagtctttcctctctctcctgggtttcctttctccagcgttcttctctttctgtctgtgtctctctcttattaaaaatacgttctgcttccttcaacaaatcttggagtgtatatccctgcaaattttctagcctttgaagcttgtttcttatgtccggggctgactgccaaataaaggacatagaaacgctggtggcctgccctggatcttccggatcatagggagtatacatcctatacgcttcttttagtctctctagaaaagtggcgggcgattcctccgccccctgtattacctgctttacctgggccaaattggtgggtgggtgtcctgccccccggagacccgctacgagcaactggcgatagagacgtagatgggtcctacctgcttcagtggtaaaatcccattcaggtctttcaagagggcaagccgcatcgatttcattgggcagctgggtgggctgcccgttggctcctgggacattctttcgtgcctctagtagaacgcgctgtttctcctccgaggttaagagaacctgcaaaatctgttcacaatcatcccaggtaggttggtgagttgtgaggaccgattctatgagagatgtcagcctcacagggtctgcagagaaaggaggattgttatttttccagttatagaggtccgaggctgagaatggccgttcggtccagttctgaggggcagagtggttgaatctggagcgggctgctccctgcgacctctcagtcggtgagccattggggaagggtcaggtgggggagctggggccacctccaccgaggcggcgggggccgcttctgcctctggtgggggcggtggcggtgctggatatggcgggggttcctcagtcagtaagtcaaccaacagttcttctcccgggggagtacctcaggtgtcttcttttcttcagctttagcgtcttttaccgctgtagggtaaaggttggaatggggaggagggtagggaggagtgggtgtcgagggattgggaagggtaggggccgagggagtgggtcggctggagcagttagagggggaaaggaaggaggggcccttggggtgtaagaaaggacgtacccagggggagggtcctgaaccaaggcctcccaagtgactatgtaatccacctggtcgggatgcccctgtggtccaggatccatagtcttctctttcacctgaaatatagtttgggggttaaaggtaccatctcgcggccagcccacgtcaaaggttggccattctgaggaacaaagagtaatccatttgcgtttatgcacatcaacagactggttatgagcataatcctgcacatctctccagtgcgagagtgttagggacaaaggagtggtgacagtttgccccatagtttctagacctaggaggacacaaatgatggttacaagacaaagaccgagcaacacagaacaggcttttgcggcgcggtttcgacacaaggtcgaaagtaaaacttcagatggggccgaagaagggggcctcccttcctcgtccccgatgaggatagcaaatccctcaatctgaggggcttccccaaacagattgggaagatgcctaggcatctaccctagtcagaaaaaagagtgagcccactgaggctctagacgctaccaggacgtctcctggagccgtggtcgggagtctgaactcttcagtttttcctcggagccgccagatacagatgtacgcagctgtgtactacagacgcaagcgcagttcataagacagattcagacagacaggcaagacaagacagacaagacagcggatccgcacaacgcttacctcccagacgtgggtcggtgatccctgggcggggggtttcgatcccggacgagcccccaaatgaaagacccccagaagaggtactttcacagaaggagacccgtacccaggaatcagcgagaccatgaacttggatgcaaacagcaagaggctttattggatacacgggtacctggggcgacttagcttctgtgaggccaagcacgccgagccaagggagaggggtccttatatagcaaaaagcatagtacagaagcgaaaagcatagttacagggactctattggacaattcaaatgaggcacagagttatttcaggttagcaagttctcaaggtctggtaatcagacgaagctgacttggctctatcttgggtacacagtggttacaacagacccggcagttcctccctccgcgtccagatggctgaccacagatatcctgtgtggcaaacaagtagtcataacgccctgaccttctattctcagaaatggttctgggtggtcttggtctcttggtctgtaatttagctagcccaagagagagagagagagcttaaggggcaggggtctttcaccctctttaggtcagttgtttgaaaggctgaccattcccattagcacaggttccgagccggactgcctttaggtgttcccatccatcttgttggcctcgacccctcaaatccttaaaatggtcaagggaaaggctcaggggagtgggagggtggctgggcttctggtcattattccagggtattccaaccacaacaacagggcaaattcagggacgcacacacagtcacggacaacactggtggctgagtgcaaactctgaaatgggataccggattgatggttggaagttgtggcctgctcaatctttttggagggactaaggggatccagttctccttggaaagacctcctggcccactagcagagtccctgtggcctaccccattctcccagccagtgatcttccaggatgtcttaaatcttagcactacacctgaccatgaagtaacagataaaacaagcggccgcatatttaacagacaatcacaaaaagacactacaccagacaatgtaccagcacagtgcacgaaaaagaaaactttacttattgactacctcaaacatacaaactaaatcaaactcctcaaatcagataagtcaaataaaatattttgaatcttgtaaacctcagacacaaatcaaaccaactggaaaagagaggctcagatacctcaaatacataagctcagctggaaaagagaggctctttcagccagcttacacaaaatatgaacaaaacaaacagctgtatcaagtacaaagtcaaatacaacgaggatctacccccacccccaaaaaggaccccagggtatccccagtcttctgacttgtgttataaaaagagcttcataggttaaatataatacaggttttacaaaatacaaaattttgcctgtcctcccaagatgttatagtggatgggtagcaggagatacctgcttaagttcccaagccaaaagaagaaaaaagaaagtgatttacctgttattagttggtgcctgaagactcagtggagggtagtaatggtgggcttggggtttccgaccaatgtaccaaatgttacacccaaaatccagaacccaaatcaccaggagactcgctctgatgcaaaagcaaagagagtatttgagttaacttagacccagagggaagtggtttttacacaggaaaatgcttggggaattccaagcctgcagggttggaattggctgcctctctgggctaaggtccacaaaatgtcaaagtatgtacctaggctaggcgttactactagcctaggcttaaagtctatggggagatgtgcctctagactagtggttactactgcttcactatttccctgggccagaaagatcaccagcctaggttagagattactactacttccctactcagggattggtcagtttttcctgctaagggattagctggttttgtgttgggctccgatttcaggagcaaagtgttttctgttggcactagtttcagagttagggtgtttccccctgactctaggttcagggataaagtgtcactcacacacacaggaatacagagtggccggcagaagacagttataaatactagcctcagctatgtaccagtttcagaaaggatcactgccatttggttaggagtgtgtgtgtatgtattgaacaggtaatccatttctttctactcctattcctggattatgtactataaagttagttgagttggtatcagaaatgtcagaagactaagtattcttcaaaggatcttgtcacctattctggggaaggaattgactggtttttggttgtctgatgttctatcacattaggaagaattatggtcttattatctttatttggagattaagtatgacataaagagatatgttgggagccaagttcacaaatgattaagttttattgtcaatttgacagggttgagatgcctgggtgtgtctgtgaggacatggctaaagaagatgggaatgtggg

SEQ ID NO: 24 - DNA, 10773 bp, Cricetulus griseus
  misc_feature: 000506F340000-360000__Type_C
aattgttttggggggttgggaggttgttgtttttgagagccttactatttagcccaggctgggccttgaactcatgatcctcctacttcaaccactgagcatgatatgtaaattaaaatataagtatattcaaatcaatcatgcactcaaattttaattcaaatttagacctataaagaagatcagaaataattctctcctttgttcattttcacttaagtaatctttggttaactcttcaatatacaatataatctatagcaacatatgtagatatgaattaagacaagaccttagcttacattaaaaagttatacatgtgccagttggtggttgtgcgagttctaggacagccaggtgtacacagaataatcctgtcttgaaaaactaaaaagttatacacttccctggcaccaaagttttttttttcccaaatgacttttcaacttgacagtttcactaccttccattgtttctctaaataaacaatgcagctggaagtcaagtgatatacttacatcactttctatccacaagaatgcaaacacaggtgttgatctaaatgaaactaccaaatatactgtgaaaccctgtccttgacagttttctcctgtggggagtgagaccccaacccccaaatctcaaattggaattggagcacggttttaaaaagagtgccacaagcatgagcagtttaacagttcacttaggacctgtgcctccaagataagcagatctttcgggtaccacaggtatgggtatgcttgcaagaacaggtgcagagtacaaatgtatgtatttgaaaggtctatggtcctgactactatcagggaatggacagcgcactcctggaggctggggatagcctctgggctgactcacataaccccgaaaggtgctgaaaatcaagaaacaacaaggcgctggtgcaagcagttgttctcgtggacggaaagagtgcgatgccaaagacactttaactgcaggtggagagtatgaggcagaaaacaccagtaagagaaagcagagcgtcactgaaagacagccagtctttcaaattaggcgcagtcagagaagccagccggaggcccaagccaacctcaatcacacacacaccgacctagtgggcaacgggggaaactgaggcggcggacaccagcaaatccccaaacaagaaataaagagaaactggctttgctgggaggggagagacctcgcctaaggcagcagctgcagggcttgcgagacagacacccacccagacaaggggaaccggttagggcaggggctcccggagggtagggcccgcgcatgcgccatctgaacgccgtgaccgctttcccacacccactctacgctcccctcagaactcggtccgtcgagggagtccccctcccccgcaacccgaccccacccggcaggaggagaaggcaacagggcttcatccggggcggtggaagtctccccgcctccggggtcggaagaccagggtgagaggaggggaacggcgaccataccagacctgctgccgccgagcgccgtccgggaggctccgccgcttgggcctccgcaccgccctccctctcctctcccgccccacctgctcacgtgaagcgactccttccgccctccggcccgcctcccaccccacctcataggatccgcgtcctcccgaggcggagcttccttccccgcagccctgcctgggcttggagcatgtcggaggcctgcgcctgcgcctgcgcctgcgcctgcgcctgcgcctgcgcctgcgcctgcacctaggctcaccctccactacccactacagcggctgcagcctaagcttgcagtctggaggtgtccctgcgttcactcttagggagcaagttctaaatttgcaaggcagcacttaaagcccacccaactcccccaacagctaccgaggcgcgcagaaccagggattgtgataggtggtttaataaaaacatgtctctcatttcaagattacgtgacagaatccgatctacaaatcagacttcagattccctagagttacctggtttcgggcactgaagaaactaggaacgaataagctgtttcaaaccgtgactcgaagacgacaattttatttgaaaagtattacaaatttaatacaaagcaatagcaataaagcaaagtttagggttcctgttcggatgaatacacaatgtatccgagaacaaaagaggtgttgtgcaagtttttaaaaaattattttgttgttgctactgaggagtgaactcaggacctcatgcatttaccacgcagctataaccgcagacttatttttaattttgagacagggtctcactgagttacccctaggctgcctttgtgaagcaagcaagccttgaatttctcatcatccagtctcgacttcctaggaagctgggattacaggtctttgtcaccaggccagatattgccaatacacaagacttagtaatttctacctctagagctgctaacttgttatgaaagaccccagaagaggtattttcgcagaaggagacccgtacccagaaatcagcgagaccacgaacttggatgcaatcagcaagaggctttattggatacacgggtacccggggcgacttagcttctgtgaggccaagcgtcccgagccaagggagagggttccttatatagcaaaaagcacagtatagaagcaaagagcatagttacagggattctattggacaattcaaatgaggcgcagggtcaatttaggtcagcatgttctcagcatatgaaaatcagataaagctgactaggctctatctagggtacacagtggttgtgacagacccggcagttgctccttatgcgtccagatggctgaccacagatatcctgtttggcaaacaagttgtcccaacgccctgaccttttattctcagaaacggttctgggtggtcttggtctcttggtctgtaatttagctagcccaagagagggagcttatagggcaggggtctttcattcccccatttctcttgtaactgaatggaatctttcattcagaggtccctggatattctggatctatttgctttagctggtggtattgttgggttaagactaaggcctgtatggcagatagtcgttctctgatgaattgagtcatcttatttagaatgcagggactaaatatgaggagtaaaaatagaataatcagaggtcccatgagtgtggatacaagggtagtgaaccagggggacttattgaaccatccctcgaaccatccttgttgagattcaaatagctgctgcctctgcttaagtctttctctaagtttggccatgctgtctctaactattcctgtgtggtctgcatagaagcagcattcttccttgagggcagcacatagtcccccttcttgtagaaataataaatctaatcctcgtctgttctgcaggaccacctcagagagtgatattaaggatttctccagggcgctgactgactcttctagggcctggatatttgtatgcatggcttgttgtagaagtttgaaatgaccagtttcctgcaaggcaaccgcaccagtccctattccagcggctactgctcccatagttattcctcctaataatagggccacggttaaggatattggttcccgtttatgtcgggttgatttctctagtacattgtaaacatattcaggttggtggtatgtgaccttgggccgaagttctattaatatacaaaagtcaatggttgcattgaggacagtggtagagacgcagggagtcaaaccagtactgcaagcccaataagttccataaggggcaataaggtatcggctgtcttgagataatggctctgtttggttacataactcttgatgtgagggaggaaccttgcctatgcatagtccctttcctgagacctcggacattgtgagcttgtgctgcatggcagctccacaactggtaggcactgaggtctggttagaatagctgcctagtactgctataccttcataatatgggggctgggaaactaggcatagccagcattcctgagtcttgttggggtctgagaaatttaaggcttggtagaccccttggactaattgtaataatctatctccagtacccaggggggtccctggttatctcggtgattggggcgtcttgggaagaggaggagataacaggacctggggtaggagtgacagctttagggatggtaggggtttagggacttggaattgggttggggccctctggtccgctattactgggtttggcccgactgattgcttaggggagtaacttgccggtacagggagaacaaggtaaccggatctttccctgctcgatatagccggagtccccagactttaggactgtcccaagtagccctttttccggcatctgtgaaccttaggatgagagggttacatcttcctccgggggtggcgcctctaatgtttcccctttggtactgtcataacagggtccacagattttaagcccacatttccgtgatcctgtctctatataccctgggatttccctccgtttgagggtgatgaagtcccaagaggaggagggtttccaataagcatctcctgtggtctcacatccccagcttgcacaatatccctcctgtggccccccgcatttatcggttggtttatgaccggggcagacataaaaatcctttcctcttgttctcatccttcccccagggtgatggcatccgtaccctgggaatggttcctggtccgagggattccaatcagaccccactaagtcacataggtcgacatagagaggagggaacccatcgtgtagagtccctatataagtgctgaggttggctacttccccggtccctagattggctattttccaggtaatattgtaaacccggtggggactggtagtttcatgggggataaacagacaaacaatcaatagaagggtgtacgagagagtcttatctttaaatgattttgagtgcggtgcgcggtccattctaatgcagtggctgaatcagtgggggtggtaagcaaaacgatgtagggtcccttccagcggggttcgaggttcttggactggtgacgccggacccagatggtgtccccgatctggtaggaatggggaatggtaggatggtccctctggtctttataagcttgagcaaggggtttccagacctcccgttgtactagttggagggcctgtaaatgagcttggagagaaggggagttagcaaaatctgagacatcttgatcaaggaagttaatggtaggggtaggtactccatgcaggatctcaaaaggtgtgagcccatgtggcccaggggtattgcgagcgcggtagagtgctagggggagtaggaggacccagtctctagtgccagttgcaagcgacaattttgttaaagtctccttaattgttctattcatcctttctacctgtcctgaactttggggtctataagcacaatgtgatttccaatcaatccccaacaaggcggccactgactgacttacctgggagacgaaggcgggcccattgtctgtccccagtacctgaggcatcccataacggggaaagatttcttcaagcaatttcttagtaacgatcttggctgtttcgtgtttagtagggaaggcttcaacccatcctgagaaggtgtcttctgccaactttagagcctgcgtgagtgcaatgagctctgccctctgcgcgaaggttccaggagggagcgcctgggcccagacgacctgatcctctgaggtgaccgccgcaccagccctccgttctccttgatgcaaaaagctgcttccatccgtgaaccagatgtggtccgggctgggtagaggctggtcagtcaggtcgggtcttgtcccatgggcctcggccagtaccggtctcgagctggagctgccgatctgtggtccaacggtactgctttctgcctgtagccccctgtacccatgcagtccggtgactcaggggtccgggtgactgattcagaacggaatgttgtacccctgtatcgactaggaaggtgactggatgccccccgactcgcagtgttatcctgggctcgggggggggggggggggggctcctggccccgacttccctaatcttctaggtttaggaggttggaggtcctgggccatggaggctgggcccgggacgtcttcttagggcaatctcttgcccaatgtcctttttccttgcagtaagcacattgatccctgtccaggtggggcccccttctgtcatccctctgcctattctgtctctgacctgtcactactgtggccaatagcctgctcatctctctattccgtttacggtctctcgcagcctctttctcctctgcttcttgtctcagtctttcctctctttcctgggcttccttcctccagcgttcttctctttctgtctgtgtctctctcttattaaaaatacgttctgcttccttcaacaaatcttggagtgtatatccctgtatctagcctttgaagcttgtttcttatgtccagggctgactgccaaataaaggacatagaaacgctggtggcctgacctggatcttccggatcatagggagtatacatcctatacgcttcttttagtctctctagaatagcggcgggcaattcctccgcccctgtatcatctgctttatacctgggccaaattggtgggtcggggtcctgcccccgagacccgctacaagcaactggcgatagagacgcagatgggtcctacctgcttcagtggtaaaatcccattcaggtctttcaagagggcaagccgcatcgatttcattgggcagctgggtgggctgcccgttggctcctgggacattcttccgtgcttctagtagaacgcgctgtttctcctccgaggttaagaggacctgcaaaagctgctgacaatcatcccaggtaggttgatgagttgtgaggaccgactctatgagagatgtcagcctcacagggtctgcagagaaagaagggttgttattttacagttatagaggtccgatgctgagaatggccaatactgaggttggccattcggtccagtccggaggggcacagtggttgaatctggaacgggctgctccctgcgacctctcagtcggtgagccattggggaagggtcaggtgggggagccggggccgcctccactgaggcggcgggggccgcttctgcctctggcgggggcggtggcggtgccggatatggcgggggttcctcagtcagtaggtaatggggaggggggtagggaggagtgggtgtcaagggattgggaagggtaggggccgagggagtgggtcggctggagcagttagagggggaaaggaaggaggggcccttggggtgtaggaaaggacgtacccaggggggagggtcctgaaccaaggcctcccaagtgactatgtatgccacctggtcgggaagcccctgtggtccaggatccatagtcttctctttcacctgaaatatagtttgggggttaaaggtaccatcctgcggccagcccacgtcaaaggctggccattctgaggaacaaagagtaatccatttgcgtctacgcacatcaacagactggttatgagcataatcctgcacatctctccagtgcgagagtgttagggacaaaggagtggtgacagtttgccccatagtttctagacctaggaggacacaaatgatggttacaagacaaagaccgagtaacacagaacaggctttcgcggcgcggtttcgacacaaggtcgaaagtaaaacttcagatggggccgaagaagggggcctcccttcctcgtccccgatgaggatagcaaatccctcaacctgaggggcttccccaaacagattaggaagatgcctaggcatctaccctagtcagaaaacagaagtgagcccactgaggctccagacgctaccaggacgtctcctggagccgtggtccggagtccgaactcgtcagttcctcctcggagctgccagatacagatgtacgcagctgtgtactacagacgcaagcgcagttcataagacggattcatacagacagacaagacaagacaagacagcggatccgcacaatgcttacctcccagacgtgggtcggtggtccctgggcggggttctcgatcccggacgagcccccaaatgaaagaccccagaagaggtactttcgcagaaggagacccgtacccagaaatcagcgagaccacgaacttggatgcaatcagcaagaggctttattggatacacgggtacccggggcgacttagcttctgtgaggccaagcgtcccgagccaagggagagggttccttatatagcaaaaagcacagtacagaagcaaagagcatagttacagggattctattggacaattcaaatgaggcgcagggtcaatttaggtcagcatgttctcagcatatgaaaatcagataaagctgactaggctctatctagggtacacagtggttgtgacagacccggcagttgctccttatgcgtccagatggctgaccacagatatcctgtttggcaaacaagttgtcccaacgccctgaccttttattctcagaaacggttctgggtggtcttggtctcttggtctgtaatttagctagcccaagagagggagcttatggggcaggggtctttcagttatccaaacaaagcacactcttttcctaatgagtcatttacaacatgaaccagaaaagcagtaatactaaaaaaaaaagttttgttgctacagttcttctttgcatctggttctttacaaagcacctacactacagtgggacaaggatgatgtttcttttttcctcatacacacctattttaaagtatgcaaataatatgaggaagaaagcataatcttgggaactcctaacaaaatgtctactattttagtagccaactttcatataatccttgattcataagtatagtaaaatcacatgtataaattaaggtttcttacataagattttattatataatcagtataatgtttggagaaaatttaaaaaaatagaaacaagttaaaaaaataggaatgtcatattttcaccaccaatattaatagttcagttaacaagattggtttagtaatccgatgtctttttttgacaaagggtctcatgtaacccaggcttgcctcaaactcttagtcctcctgtctccatctcccaaatactggaatgacataccagtcagtaatctcccttttcaattaacaatacataccttatttctgattattacatctaattattagaccatgaattgcctctagttatgccctttaggtgtttgtttaactgtgcaatctgaagatgaatggctctttgcagatatctgaaaaactgaagtacaaaattcctggatttgaataaagtttacttcaagaagtctacagtgaatcatttagtttgtctttttcttttttcttttctttctttttttttttttttttttttttttttggtttttcaagacagggtttctctgtggctttggagcctatcctggaactcgctcttgtagatcaggctggtcttgaactcacagagatccacctgcctctgcctcccaagtactgagattaaaaacttgcaccaccaccgcccagctagtttgtcttctaaaccctacatttttgcaaactagaagctgtatcagaaagttcaatggatttgagacaaatactctggactaatacatgtcacttagggtgttgccttagacttgtttcctggtattagatatgatgcaacttatttagtacatagtattacttgttgtgtatagtatgggatgttgatactgaaatggtaaggatgcttgtttatagactctcctggactcttctttcactctctctgtatctcatgtattcccacctagcctcaaattcaatataaagcagatgatgaccttggacctctgatcccattagttacaagtatacactaacacattcagtgttatatgtgatttggggcatcaaacagggtttcttgcctgaaaagtaagcactctaccacctaagctaaatcctcagcccctatagaatctttttactgttggtgttttgcgatggagtttcatcatgtaactactttttgttttgctttttgtttttcaagacagggtttcactgtggctttggagcctatcctgccactggctctcatgtaactactttttaaaaacagatttttattttgggcagtgatggtgcatgcctttgatctcagcactcaggaggaagaggcagggggatctttgaattagaggccagcctggtctacaaagtgagttccagtacagccagagctatacagagaaaccctgtctcaaaacaaaacaaaacaaaacaaaaaaccctagccaggcgttggtagtgcacgcctttaaacccagcactcgggaggcagaggcaggtggatctctgtgagttgaagaccagcctggtctacaagagctagttccaggacagcctccaaagccacagagaaaccctgtctcgaaaaaccaaaaaaacaaaacaaaaacaaacaaaacaaaaaacccaaaacagattttacatactctattttaatgcctaccaatgtcacatagtaactatttcacagaaaattaattgagggtttaattatgcttaactattaccaacaggtggctttgaaaccacaaatcatataagtattaaaatcatataagtagaatttggg

SEQ ID NO: 25 - DNA, 2124 bp, Homo sapiens
  misc_feature (1)..(2124): Homo sapiens MRE11 (NM_005591.4)
atgagtactgcagatgcacttgatgatgaaaacacatttaaaatattagttgcaacagatattcatcttggatttatggagaaagatgcagtcagaggaaatgatacgtttgtaacactcgatgaaattttaagacttgcccaggaaaatgaagtggattttattttgttaggtggtgatctttttcatgaaaataagccctcaaggaaaacattacatacctgcctcgagttattaagaaaatattgtatgggtgatcggcctgtccagtttgaaattctcagtgatcagtcagtcaactttggttttagtaagtttccatgggtgaactatcaagatggcaacctcaacatttcaattccagtgtttagtattcatggcaatcatgacgatcccacaggggcagatgcactttgtgccttggacattttaagttgtgctggatttgtaaatcactttggacgttcaatgtctgtggagaagatagacattagtccggttttgcttcaaaaaggaagcacaaagattgcgctatatggtttaggatccattccagatgaaaggctctatcgaatgtttgtcaataaaaaagtaacaatgttgagaccaaaggaagatgagaactcttggtttaacttatttgtgattcatcagaacaggagtaaacatggaagtactaacttcattccagaacaatttttggatgacttcattgatcttgttatctggggccatgaacatgagtgtaaaatagctccaaccaaaaatgaacaacagctgttttatatctcacaacctggaagctcagtggttacttctctttccccaggagaagctgtaaagaaacatgttggtttgctgcgtattaaagggaggaagatgaatatgcataaaattcctcttcacacagtgcggcagtttttcatggaggatattgttctagctaatcatccagacatttttaacccagataatcctaaagtaacccaagccatacagagcttctgtttggagaagattgaagaaatgcttgaaaatgctgaacgggaacgtctgggtaattctcaccagccagagaagcctcttgtacgactgcgagtggactatagtggaggttttgaacctttcagtgttcttcgctttagccagaaatttgtggatcgggtagctaatccaaaagacattatccattttttcaggcatagagaacaaaaggaaaaaacaggagaagagatcaactttgggaaacttatcacaaagccttcagaaggaacaactttaagggtagaagatcttgtaaaacagtactttcaaaccgcagagaagaatgtgcagctctcactgctaacagaaagagggatgggtgaagcagtacaagaatttgtggacaaggaggagaaagatgccattgaggaattagtgaaataccagttggaaaaaacacagcgatttcttaaagaacgtcatattgatgccctcgaagacaaaatcgatgaggaggtacgtcgtttcagagaaaccagacaaaaaaatactaatgaagaagatgatgaagtccgtgaggctatgaccagggccagagcactcagatctcagtcagaggagtctgcttctgcctttagtgctgatgaccttatgagtatagatttagcagaacagatggctaatgactctgatgatagcatctcagcagcaaccaacaaaggaagaggccgaggaagaggtcgaagaggtggaagagggcagaattcagcatcgagaggagggtctcaaagaggaagagcagacactggtctggagacttctacccgtagcaggaactcaaagactgctgtgtcagcatctagaaatatgtctattatagatgcctttaaatctacaagacagcagccttcccgaaatgtcactactaagaattattcagaggtgattgaggtagatgaatcagatgtggaagaagacatttttcctaccacttcaaagacagatcaaaggtggtccagcacatcatccagcaaaatcatgtcccagagtcaagtatcgaaaggggttgattttgaatcaagtgaggatgatgatgatgatccttttatgaacactagttctttaagaagaaatagaaga

SEQ ID NO: 26 - DNA, 7713 bp, Cricetulus griseus
  misc_feature (1)..(7713): Cricetulus griseus PolQ (XM_003501093.4)
atgagtcttccgcgccggagtgggaaacggcggcgctcggcgtccggctccgactcgttctcgggagacggtgatagctgcgccagccctcagctgccgtctaggccggtgctgagtccaccttcgcggctgggacgcggcccgagagccgcgggtgcagggacatgcaagaaaagagttcctgatgaccagatagaccaactgctattggcaaattggggagatcctaaagcagttctggagaaataccatagttttggtgtaaaaaagatgttcgaatggcaggcagagtgtcttttgcttggacaagtcctggaaggaaagaatctagtttattcagctcctacaagtgccgggaagactcttgttgcagagttacttattttgaagcgtgttttggaaatgcggaagaaagctttgtttattctgccctttgtgtctgtggctaaagagaagaaatactacctccagagtctgtttcaggaagtaggaataaaagtagatggctacatgggtagttcctcacctactggacgcttttcctccttggacattgctgtttgcactattgagagagccaatgggctgatcaatcgcctcattgaggaaaataagatggatttgttaggaatggtggttgtagatgaattacacatgctgggagactctcaccgaggatatcttctggaacttttactgaccaagatttgctatgttactaggaaatcaacattgtgccaggcagattcagcccgtgctttgtgtaatgctgtgcagattgttggcatgagtgctactcttcctaatttgcagcttgtagcttcctggttggatgctgaactttaccatactgactttcgccctgtaccacttttggaatcaataaaaataggaaattccatatatgactcttcaatgaagctcgtgagagaacttcagcctgtgcttcaagtgaagggagatgaagatcacattgttagtttatgttatgagaccgtttgtgataaccattcagtattactcttttgtccatcaaaaaaatggtgtgaaaaggtagcagatatcattgcccgagagttttataatctgcaccatcaacctgagagattagtaaaaccatctgaatttccaccagtgaatctagaccaaaagagccttctggaagtgatggatcagctaaaacgttcgccttcaggactagactccgtattgaagaacactgtgccatggggagtagcatttcatcatgcaggtcttacttttgaggagagagatatcatagaaggagccttccgtcaaggtcttattcgggtcttggcagcaacatctactctttcttctggagtgaatcttcctgctcgccgggtgattattcgcactccagttttcggtggccagactttagatatcctcacttataagcagatggttggccgtgctggcaggaaaggagtagacacaatgggtgaaagtatcctagtttgtaagccctctgagaaatcgaaaggcgttgctctgcttcaaggatctctggaacctgtgcatagctgtctgcagagacaagaagtcactgctaacatgatacgagcgattctggagataattgttggtggagtggcaagtacatcacaagatatgcagacttatgcctcctgcacattcttggctgctgctgtaaaagaagggaagcagggaattcagagaaatcaagatgatgttcattttggagcaattgatgcctgtgtgacatggctgttagaaaatgagttcatccaggaggcagagcccagtgatggctcaggaggaaaggtgtatcacccgacacatcttggatcggctactctttcttcctcactctctccaactgataccctagatatttttgctgacctccaaagagcaatgaaaggatttgttttagagaatgaccttcatatcgtctacctggttacacctgtgtttgaagattggactggtattgattggtatcgatttttctgtttatgggaaaaattgcccacttctatgaaaagggtggcagaactggttggagttgaggaagggttcttggctcgatgtgtgaaaggaaaagtagtagctagaactgacagacagcatcgacaaatggccatccataaaaggtttttcacaagtcttgtgttattggatttaatcagtgaaattcccttaaaggaaattaatcagaaatatggatgtaatcgtggacagattcaatctttgcaacagtcagctgctgtttatgcaggaatgattacagtgttttccaaccgtcttggctggcacaatatggaactactactttcccagtttcagaagcgtcttacctttggcatccagagggaactgtgtgacctcatccgggtgtccttactgaacgcccagagagccaggttcctgtatgcctctggctttctcactgtagcagaccttgccagagctaatgttgcggaggtggaggtggttctgaagaatgctgtgcctttcaaaagtactcgaaaggcagtagatgaagaagaggaagcagccgaagaacgtcgcaatatgcaaaccatttgggtgacgggcagaaaaggtttatctgcaagggaagcagcaaccttaatagtggaagaagccaaaacgattctgcagcaggacttacttgaaatgggagtgcagtgggatccaaattcatccttgagttctagcacatcttcactgaccagtagtgagtcagaagtaaatgaacgcacacttcaatctcaaactaagaattctcataaaaagttgacatcaaagaatagaaacagtgtgagagcaagtgtttctaatgataaaccgtcaccagatacagcacaaggcttaggtgaacacagcgagcatacagactccctctgtttgttacaggggaacaaacaccagcaccagccacattctgtttgcagagcaagaaaacggacttctttgggtataaacaaagaaaagcttcggatgtctctgaatggaggagaaccaagcactaaggaagttcttcaaactttctcaatggagaaaacaaggaaagctgctttgactgctaattcagagcagacaaacaccagtttcccatcttggagagacagaaagcatcgaaaaaaatcttggggcagtagcccagtgcgtgattctagggctaatcatcacagagatgatttccaggaacacactgtgtctagatctactctatgtgaagagccactttccttagacaagcagaatatagaatttagaagttcagggctacttataaaaaatgcatctttttgtgccaatgaaaaatacaacaaaacctcattctctttacaaatgcaacagccttgcctaaggaaaaaaacagaaagtactggtgctgtggaacattcctttgcagaatctcagagtaaaaatgtgactggtcagtcccctggcgttgctagcaatggaagaggattggctgatactgagacaggaaagataaatgaggtactcatagagaatggtgcagaaagtcagaatgtttctgtgaaacaccatgacacccatccaattagccagtgcctggaaaatcagtgtgacaaacagacaaacacttgcactaagcggaaagcattaatagagagacaagtgtcctgtgaagcagttagttacatggccagagactcaaatgatgtttcaactatcaactctgaaagcataaagcttcatagcaaagacgatgagtcaaatcactgtcaggtattgggaaataatacgggcagaagtgaggcacctcgtggattactgcagtcagctgcagaattcagtcaagcagatggccagcatgagcatcttctaaattcttctggaatacaagaaaaaacagatgcttatgccacaaataaaactgaacataatcatgtttctaacttagccccctgtgactttggagatagcttctatctggatactcagtcagagaaaataattgaacagttggcaactgaacatgccaagcaaagaacaaaggcggtaacagcgaagggctctgacactcggaactcagggagctcatttcagaacaagtgtcacagtactcgaggtgaacagcacttccaaagagcagccaacacagaccatttggacagtaagagtgtagagaccacaaaacagaaccctgaaaagagcatcggtagactaactgcagaaagcatcatcttccactctccaacaccacagggggaaaatggcccttgctttagagtgaatgagcagtctgttactgactcccagctaaacagttttcttcaaggttttgaaacacaagaaatggttaaaccagtcctatctctagctcctccagcgggaactcctactggtttggaggaagaaaatctgcctgaaaccagtttgaatatgagtgacagtatactatttgacagttttggtgaagatgacttagtaaaaggacagtcacctgatgtacaggcaaagcagcccctcctttctgtaatgacaccaaaccatctcagcagctctctgtgcccacgagaagacccagtcatgaaggcaaatgtgaatgaccatcagggtattcagcagccggagacttgttccagtggggaatctgttatattttcagaggtggattctgctcagatgattgaagccttggacagtgtggctgcattacatgtccaacagaattgtaactcagtaacccttaagactttagaactcagtgattctgcaatgctggataatgagtgtccccaaggaaaagtggccagaggagataaaagcgaaagggcccaaatgtccaaactcactgagacaaatcaagataattcaatcacttggtcaggagcatcatttgacttaagtccagaactgcaaaggattttagacaaagggtctagtcctctagaaaacgaaaggcctaaattaacacagacaaacttgtcttgctttgaaagaaacggtacagagttaaatgagagacaggaaatgaatccaaatttggaggccgttcaaattcagagaacttcacttttccccaataatggagttcaaaacaagattgaggggatagggaacgacaccaggcgtggtgaggccttatatccctcagctcgtaaagaaagtgacacagctgatgacaatggcctcattcctcccacacccattcttgcttctacttctaagctgtcatttccagaaattctcggaacatctgtaaaccatctgaaagctgacagtgtttttctgccaggtgaaagttgtttatttggctcaccttcagatagtcaagagcgtagagacagcttcaaagatgaccgttcagttggagacacaagtttttctctgcagttctctcaggatgaactgcagctaactccagcctcatgcagctccgaaagcttggccataattgatgtagcgagtgaccacacgctgtttgagacgtttgtgaaggagtggcgatgccaaaagcgcttttccatctctctggcttgtgaaaagattagaagtccgacatcttccaaaaccgctaccatagggggaaggttgaagcaagtgagctcccctcagaagacctctgctgaagatgatggctttcctgtccatggctctgatggtgtcatagtagttggactagcagtgtgctggggtggaagggatgcctattatttgtcactgcagaaggaacaaaagcattccgaaattagccccagtttggccccacctcctctggatacaacattgactgtggaagagaggatggagcaccttcaatcctgcttgcaaaagaaatctgacaaagagcagactgttgtcacctatgacttcatccagagctataaaatcctcctcctgtcctgtggtgtctccctggaaccaagttatgaagatcctaaggtggcatgctggcttctcgatccagactctaaggaaccaactctccatagcatagtgaccagtttcctccccgaggagcttcctctgctagaaggaatagagacaggccaagggattcagagcctggggctaaatgtggacactgagcattctgggcggtacagagcatctgtggaatccattctcatcttcaactccatgaatcaactcaactctttgttgcagaaggaaaaccttcacgatattttctgtaaagtggagatgccctcccagtactgcctggccttgctagaactgaatggaattggctttagtactgcagaatgtgaaagtcagaagcatatcatgcaagccaagctggacgccattgagactcaggcctatcagctagctggtcacagcttttcctttaccagtgcagatgacatcgcacaggttttatttttggagctgaagctaccaccgaatggagagatgaaaatgcaaggcagcaaaaaaactttgggttctaccagaagaggcaatgagaatggccacaaactaaggctgggaagacagttcagcactagcaaggatgttttaaataaattaaaggccttacatcctttaccaggcctgatactggaatggagaagaatcagtaatgctattaccaaagtggtcttccctctgcagcgggaaaagcgcctgaacccttttctcagaatggaaaggctctaccctgtatcacagtcacacacggccacaggacggataacctttatagaacctaatatccagaatgtgccaagagattttgagattaaaatgcccacagtagtaagggaaagcccaccttctcaagctccaggcaaacgcctgcttccaatgaccagaggacaaaataagaagttttatggcttgcaccctgggaatgggacactgatggaagagaaagcctcagatagaggggtgccattttcagtgagcatgcgccatgcttttgtgcctttcccagggggtgtaattttggctgctgattattctcagcttgaactgaggatcctggctcacctgtcacgtgactgtcgcctcattcaagtcttaaacactggagctgatgttttccggagcattgctgctgaatggaagatgatcgagcctgactctgttggggaggatctgaggcaacaagcaaagcagatttgctatggaatcatatatgggatgggagctaaatccttaggagagcagatgggcattaaagaaaacgatgctgcttgctatattgactctttcaagtccagatatacagggattaatcatttcctgagggacacagtgaaaaaatgtagaagagatggatttgttcagaccattttgggaaggcgcagatatttgcctggaatcaaagacaacaatccttatcataaagctcatgctgaacgtcaggccatcaacacaacagtccaaggatcagctgctgatatagtaaaaacagccacagttaacattcagaagcaattagagaccttccattcagccttcaaatcccatggtcatcgggaaagcatgctccagcatggccaaacaggattgttgccaaagaaaaaactgaaagggatgttttgtccaatgagaggaggcttctttatccttcaacttcatgatgagcttttatatgaagtggcagaagaggatgttgttcaggtagctcagattgtcaagaaggaaatggaatgtgctgtaaaactttctgtgaagctaaaagtgaaagtgagaatgggtgccagctggggacagctggaggactttgatgtg

SEQ ID NO: 27 - DNA, 516 bp, Homo sapiens
  misc_feature (1)..(516): Homo sapiens CIRBP (XM_006722637.2)
atggcatcagatgaaggcaaactttttgttggagggctgagttttgacaccaatgagcagtcgctggagcaggtcttctcaaagtacggacagatctctgaagtggtggttgtgaaagacagggagacccagagatctcggggatttgggtttgtcacctttgagaacattgacgacgctaaggatgccatgatggccatgaatgggaagtctgtagatggacggcagatccgagtagaccaggcaggcaagtcgtcagacaaccgatcccgtgggtaccgtggtggctctgccgggggccggggcttcttccgtgggggccgaggacggggccgtgggttctctagaggaggaggggaccgaggctatggggggaaccggttcgagtccaggagtgggggctacggaggctccagagactactatagcagccggagtcagagtggtggctacagtgaccggagctcgggcgggtcctacagagacagttatgacagttacgctacacacaacgag

SEQ ID NO: 28 - DNA, 1050 bp, Homo sapiens
  misc_feature (1)..(1050): Homo sapiens RAD51 (NM_002877.6)
atgggtagcaagaaactaaaacgagtgggtttatcacaagagctgtgtgaccgtctgagtagacatcagatccttacctgtcaggactttttatgtctttccccactggagcttatgaaggtgactggtctgagttatcgaggtgtccatgaacttctatgtatggtcagcagggcctgtgccccaaagatgcaaacggcttatgggataaaagcacaaaggtctgctgatttctcaccagcattcttatctactaccctttctgctttggacgaagccctgcatggtggtgtggcttgtggatccctcacagagattacaggtccaccaggttgtggaaaaactcagttttgtataatgatgagcattttggctacattacccaccaacatgggaggattagaaggagctgtggtgtacattgacacagagtctgcatttagtgctgaaagactggttgaaatagcagaatcccgttttcccagatattttaacactgaagaaaagttacttttgacaagtagtaaagttcatctttatcgggaactcacctgtgatgaagttctacaaaggattgaatctttggaagaagaaattatctcaaaaggaattaaacttgtgattcttgactctgttgcttctgtggtcagaaaggagtttgatgcacaacttcaaggcaatctcaaagaaagaaacaagttcttggcaagagaggcatcctccttgaagtatttggctgaggagttttcaatcccagttatcttgacgaatcagattacaacccatctgagtggagccctggcttctcaggcagacctggtgtctccagctgatgatttgtccctgtctgaaggcacttctggatccagctgtgtgatagccgcactaggaaatacctggagtcacagtgtgaatacccggctgatcctccagtaccttgattcagagagaagacagattcttattgccaagtcccctctggctcccttcacctcatttgtctacaccatcaaggaggaaggcctggttcttcaagcctatggaaattcc

SEQ ID NO: 29 - DNA, 17 bp, Artificial Sequence
  Synthetic Forward primer of the TaqMan qPCR assay overlapping the left junction of the integrated ERV C 109F
ccctgcccttccatgtg

SEQ ID NO: 30 - DNA, 17 bp, Artificial Sequence
  Synthetic Reverse primer of the TaqMan qPCR assay overlapping the left junction of the integrated ERV C 109F
cgccgacaggaggaaga

SEQ ID NO: 31 - DNA, 25 bp, Artificial Sequence
  Synthetic TaqMan probe of the qPCR assay overlapping the left junction of the integrated ERV C 109F
ccctctcccttagccctttctttct

SEQ ID NO: 32 - DNA, 18 bp, Artificial Sequence
  Synthetic Forward primer of the TaqMan qPCR assay overlapping the right junction of the integrated ERV C 109F
gctgattcctgggtgtgt

SEQ ID NO: 33 - DNA, 21 bp, Artificial Sequence
  Synthetic Reverse primer of the TaqMan qPCR assay overlapping the right junction of the integrated ERV C 109F
gcagcagaaatgaacatgtga

SEQ ID NO: 34 - DNA, 25 bp, Artificial Sequence
  Synthetic TaqMan probe of the qPCR assay overlapping the right junction of the integrated ERV C 109F
agaggcactttcgtagagggagacc

SEQ ID NO: 35 - DNA, 19 bp, Artificial Sequence
  Synthetic Forward primer of the TaqMan qPCR assay overlapping the allele breakpoint of the wild type allele
accagattgatccctgtgc

SEQ ID NO: 36 - DNA, 18 bp, Artificial Sequence
  Synthetic Reverse primer of the TaqMan qPCR assay overlapping the allele breakpoint of the wild type allele
ctgtgctacgggctgaaa

SEQ ID NO: 37 - DNA, 25 bp, Artificial Sequence
  Synthetic TaqMan probe of the qPCR assay overlapping the allele breakpoint of the wild type allele
cctgcccttccatgttcatttctgc

SEQ ID NO: 38 - DNA, 3936 bp, Homo sapiens
  misc_feature (1)..(3936): Homo sapiens RAD50 (NM_005732)
atgtcccggatcgaaaagatgagcattctgggcgtgcggagttttggaatagaggacaaagataagcaaattatcactttcttcagcccccttacaattttggttggacccaatggggcgggaaagacgaccatcattgaatgtctaaaatatatttgtactggagatttccctcctggaaccaaaggaaatacatttgtacacgatcccaaggttgctcaagaaacagatgtgagagcccagattcgtctgcaatttcgtgatgtcaatggagaacttatagctgtgcaaagatctatggtgtgtactcagaaaagcaaaaagacagaatttaaaactctggaaggagtcattactagaacaaagcatggtgaaaaggtcagtctgagctctaagtgtgcagaaattgaccgagaaatgatcagttctcttggggtttccaaggctgtgctaaataatgtcattttctgtcatcaagaagattctaattggcctttaagtgaaggaaaggctttgaagcaaaagtttgatgagattttttcagcaacaagatacattaaagccttagaaacacttcggcaggtacgtcagacacaaggtcagaaagtaaaagaatatcaaatggaactaaaatatctgaagcaatataaggaaaaagcttgtgagattcgtgatcagattacaagtaaggaagcccagttaacatcttcaaaggaaattgtcaaatcctatgagaatgaacttgatccattgaagaatcgtctaaaagaaattgaacataatctctctaaaataatgaaacttgacaatgaaattaaagccttggatagccgaaagaagcaaatggagaaagataatagtgaactggaagagaaaatggaaaaggtttttcaagggactgatgagcaactaaatgacttatatcacaatcaccagagaacagtaagggagaaagaaaggaaattggtagactgtcatcgtgaactggaaaaactaaataaagaatctaggcttctcaatcaggaaaaatcagaactgcttgttgaacagggtcgtctacagctgcaagcagatcgccatcaagaacatatccgagctagagattcattaattcagtctttggcaacacagctagaattggatggctttgagcgtggaccattcagtgaaagacagattaaaaattttcacaaacttgtgagagagagacaagaaggggaagcaaaaactgccaaccaactgatgaatgactttgcagaaaaagagactctgaaacaaaaacagatagatgagataagagataagaaaactggactgggaagaataattgagttaaaatcagaaatcctaagtaagaagcagaatgagctgaaaaatgtgaagtatgaattacagcagttggaaggatcttcagacaggattcttgaactggaccaggagctcataaaagctgaacgtgagttaagcaaggctgagaaaaacagcaatgtagaaaccttaaaaatggaagtaataagtctccaaaatgaaaaagcagacttagacaggaccctgcgtaaacttgaccaggagatggagcagttaaaccatcatacaacaacacgtacccaaatggagatgctgaccaaagacaaagctgacaaagatgaacaaatcagaaaaataaaatctaggcacagtgatgaattaacctcactgttgggatattttcccaacaaaaaacagcttgaagactggctacatagtaaatcaaaagaaattaatcagaccagggacagacttgccaaattgaacaaggaactagcttcatctgagcagaataaaaatcatataaataatgaactaaaaagaaaggaagagcagttgtccagttacgaagacaagctgtttgatgtttgtggtagccaggattttgaaagtgatttagacaggcttaaagaggaaattgaaaaatcatcaaaacagcgagccatgctggctggagccacagcagtttactcccagttcattactcagctaacagacgaaaaccagtcatgttgccccgtttgtcagagagtttttcagacagaggctgagttacaagaagtcatcagtgatttgcagtctaaactgcgacttgctccagataaactcaagtcaacagaatcagagctaaaaaaaaaggaaaagcggcgtgatgaaatgctgggacttgtgcccatgaggcaaagcataattgatttgaaggagaaggaaataccagaattaagaaacaaactgcagaatgtcaatagagacatacagcgcctaaagaacgacatagaagaacaagaaacactcttgggtacaataatgcctgaagaagaaagtgccaaagtatgcctgacagatgttacaattatggagaggttccagatggaacttaaagatgttgaaagaaaaattgcacaacaagcagctaagctacaaggaatagacttagatcgaactgtccaacaagtcaaccaggagaaacaagagaaacagcacaagttagacacagtttctagtaagattgaattgaatcgtaagcttatacaggaccagcaggaacagattcaacatctaaaaagtacaacaaatgagctaaaatctgagaaacttcagatatccactaatttgcaacgtcgtcagcaactggaggagcagactgtggaattatccactgaagttcagtctttgtacagagagataaaggatgctaaagagcaggtaagccctttggaaacaacattggaaaagttccagcaagaaaaagaagaattaatcaacaaaaaaaatacaagcaacaaaatagcacaggataaactgaatgatattaaagagaaggttaaaaatattcatggctatatgaaagacattgagaattatattcaagatgggaaagacgactataagaagcaaaaagaaactgaacttaataaagtaatagctcaactaagtgaatgcgagaaacacaaagaaaagataaatgaagatatgagactcatgagacaagatattgatacacagaagatacaagaaaggtggctacaagataaccttactttaagaaaaagaaatgaggaactaaaagaagttgaagaagaaagaaaacaacatttgaaggaaatgggtcaaatgcaggttttgcaaatgaaaagtgaacatcagaagttggaagagaacatagacaatataaaaagaaatcataatttggcattagggcgacagaaaggttatgaagaagaaattattcattttaagaaagaacttcgagaaccacaatttcgggatgctgaggaaaagtatagagaaatgatgattgttatgaggacaacagaacttgtgaacaaggatctggatatttattataagactcttgaccaagcaataatgaaatttcacagtatgaaaatggaagaaatcaataaaattatacgtgacctgtggcgaagtacctatcgtggacaagatattgaatacatagaaatacggtctgatgccgatgaaaatgtatcagcttctgataaaaggcggaattataactaccgagtggtgatgctgaagggagacacagccttggatatgcgaggacgatgcagtgctggacaaaaggtattagcctcactcatcattcgcctggccctggctgaaacgttctgcctcaactgtggcatcattgccttggatgagccaacaacaaatcttgaccgagaaaacattgaatctcttgcacatgctctggttgagataataaaaagtcgctcacagcagcgtaacttccagcttctggtaatcactcatgatgaagattttgtggagcttttaggacgttctgaatatgtggagaaattctacaggattaaaaagaacatcgatcagtgctcagagattgtgaaatgcagtgttagctccctgggattcaatgttcat

SEQ ID NO: 39 - DNA, 3540 bp, Escherichia coli
  misc_feature (1)..(3540): Escherichia coli str. K-12 RecB (NC_000913.3)
atgagtgatgtcgccgagacactagatcctttgcgcttgcccttacagggtgagcgcctgattgaagcctctgccggcacaggcaaaacctttacgattgcggcgctctatttgcgcctgttacttggactaggcggttccgccgcctttccccgcccgctgaccgttgaagaactgctggtggtcacctttaccgaggctgccacggcagaattgcgcggtcgtatccgtagcaatatccacgagttgcgcatcgcctgtctgcgtgaaaccaccgacaatccactgtacgaacgcctgctggaagagatcgacgataaagcgcaagccgcgcagtggttgttgttagccgaacggcagatggatgaagcggcagtctttactattcacggcttttgccagcgcatgctcaacctgaatgcctttgaatccggcatgctgtttgagcagcagctgattgaagatgagtctctgctacgctaccaggcctgcgccgatttctggcgtcgccactgctacccgctgccgcgtgaaatagcccaggtcgtctttgaaacctggaaagggccgcaggcgttgctgcgcgatattaatcgttatctgcaaggcgaagcgccggttatcaaagcaccgccgcccgatgatgaaacgctggcttcccgtcacgcgcaaattgtggcgcgtattgatacggtaaaacagcagtggcgcgacgcagtgggtgaactggatgcgctgatcgaatcttctggtattgatcgacgcaagtttaaccgtagcaatcaggctaaatggatcgacaagatcagcgcctgggcagaagaagagacaaacagttatcagttgccggagtcgctggaaaaattctcccagcgtttcttagaagatcgcacgaaggccgggggggaaaccccgcgacatccactgtttgaggcgatcgatcaactgcttgcagaaccattgtcgatccgcgatctggtgatcacccgcgcattggctgagatccgcgaaacagtagcgcgtgaaaaacgccgccgtggcgaattgggttttgatgacatgttaagtcggctcgattccgcgctgcgtagcgaaagcggtgaggtgttggcagcggcgatccgtacgcgattcccggtggcaatgatcgatgaatttcaggataccgacccccagcagtaccgaatttttcgccgtatctggcaccatcagccggaaaccgcattgttgctaattggcgacccgaagcaggccatatatgcattccggggtgcggatatcttcacttatatgaaggcgcgtagcgaagttcacgcccactacactttagacaccaactggcgttccgcaccaggaatggtgaacagcgtgaataagcttttcagccagactgatgacgcgttcatgtttcgcgaaataccgtttattccagtgaaatcagccgggaaaaatcaggcgttacgttttgtatttaaaggtgaaacacagcctgcgatgaaaatgtggctgatggaaggcgaaagctgcggcgttggcgattatcaaagtaccatggcgcaggtatgtgctgcgcaaatccgcgactggctacaagccggacagcggggcgaagcgttgctgatgaacggcgacgacgcgcgtccggtgcgtgcttcggacatcagtgtgctggtgcgcagccgccaggaggccgcccaggtgcgcgatgccttaacgttgctggaaatcccttccgtttacctttcgaaccgcgacagtgtttttgaaactctggaagcgcaggaaatgctttggttgttgcaggcggtgatgacgcccgaacgtgagaacaccctgcgtagtgcgctggcaacgtcaatgatggggctgaacgcgctggatatcgaaacgctgaacaatgacgaacatgcgtgggatgtggtagtcgaagagttcgatggttatcggcaaatctggcgcaaacgtggcgttatgccgatgctgcgggcgctgatgtcggcgcgtaacattgctgaaaacttgctggcaacggcaggcggtgagcggcgtcttaccgatatcttgcatatcagcgaactgctacaagaagccggaacgcagctggaaagtgaacatgcgctggtacgctggttatcgcaacatatcctcgagccagacagtaatgcctccagccaacaaatgcgtctcgaaagtgataaacatctggtgcagattgtcacgatccacaaatcgaaagggctggaatatccattggtctggctgccgtttatcaccaatttccgcgtccaggagcaggcgttttatcacgatcgccactcgtttgaggcagttctggatcttaatgctgcgccagaaagcgtcgacctcgcggaggccgaacgtctggcggaagatctgcgtttgctttacgtggcgctgacacgttcggtttggcattgcagtctcggcgttgcaccgctggtgcgccgtcgtggcgataaaaaaggtgacaccgacgtccaccaaagtgcgctcgggcgtttgctgcaaaaaggggaaccgcaagatgcggcagggcttcgcacctgtattgaagcgttatgcgatgatgatattgcctggcaaacggcacaaactggtgataaccaaccctggcaggttaatgatgtttctacagcagagctgaatgcgaagacgttacaacgattgcccggcgataactggcgcgtcaccagctactctggtttgcaacagcgtggtcacggtatcgcccaggatttgatgcctcggctggatgtcgatgctgcaggcgttgccagcgtcgttgaagaaccgacgttaacaccacatcagtttccgcgcggtgcgtcaccggggacgttcttgcacagtttgtttgaagacctggattttacccagccggttgacccgaactgggtgcgggaaaaactggaactcggcggctttgaatcgcagtgggaaccggtattgaccgagtggatcacggctgtcctccaggcacctctcaatgaaaccggcgtaagcctgagtcaactttccgcccgcaataaacaggtggagatggagttttatctgccgattagtgaaccgcttatcgccagtcagcttgatacgttaatccgccagtttgacccgctatccgcaggctgcccgccgctggagttcatgcaggtacgtggcatgttaaaaggctttatcgacctggtgttccgccacgaagggcgttattacctgctcgactataaatccaactggttgggtgaagacagttcggcttacacccaacaggctatggcagcggcaatgcaggcacaccgctatgatctgcaatatcagctttataccctggcgctgcatcgttatctgcgccatcgcattgctgattacgactatgagcaccactttggcggcgttatttatctgttcctgcgtggcgttgataaagaacatccgcaacaggggatttacacaacccgacccaacgccgggttgattgccctgatggatgagatgtttgccggtatgaccctggaggaggcg

SEQ ID NO: 40 - DNA, 3366 bp, Escherichia coli
  misc_feature (1)..(3366): Escherichia coli str. K-12 RecC (NC_000913.3)
atgttaagggtctaccattccaatcgtctggacgtgctggaagcgttgatggagtttattgtcgaacgcgaacggctggacgatcctttcgaaccagagatgattctggtgcaaagtaccggtatggcacagtggctgcaaatgaccctgtcgcaaaagtttggtattgcggcaaacattgattttccgctgccagcgagctttatctgggatatgttcgtccgggtgttaccggaaatccccaaagagagcgcctttaacaaacagagcatgagctggaaactgatgactctgctgccgcaattgctggagcgcgaagactttaccctgttgcggcattatctgactgacgatagcgacaagcgaaaactgttccagctttcctcaaaagcggcggacctgtttgaccagtatctggtctatcgtccggactggctggcacagtgggaaacaggacatttggttgaagggctgggagaagcacaggcctggcaagccccgttgtggaaggcgctggtggaatatacccatcaactcgggcaaccgcgctggcaccgcgccaatctctatcagcgctttatcgaaacgctggagtccgcgacgacctgcccgccggggttaccttcgcgcgtctttatatgcggtatttccgcgttaccgcctgtttatctccaggcgctacaggcgctgggtaaacatattgaaatccatctcctgtttaccaacccctgccgttattactggggcgatattaaagatcctgcttatctggcgaaactgctgacccgtcagcgtcgacacagttttgaagatcgcgaattgccgctatttcgtgatagcgaaaatgccgggcagctctttaacagcgatggtgaacaggatgtcggcaacccgctgctggcttcatggggtaagcttgggcgcgactacatttatctcctttctgacctggagagcagccaggagctggacgcctttgtcgatgtcacgccagataacctgctgcataacattcagtctgacattctggaactggaaaaccgcgccgttgctggtgtgaacatcgaagagttttcccgtagcgataacaaacgcccgcttgatccactggatagcagtatcaccttccacgtttgccatagcccgcagcgtgaagttgaagttttacacgatcgcctgctggcgatgctggaggaagacccgacacttactccgcgcgacatcatcgtgatggtggctgatatcgacagctacagtccgtttattcaggctgtgtttggtagcgcacctgcggatcgttacctaccttacgccatttccgaccgtcgtgcgcggcagtcacatccggtactggaagcgtttatcagcctgttatcactgcctgacagtcgttttgtgtcagaggatgtgctggcgttgctggatgtgccggtgctggcggcgcggtttgacatcaccgaagaagggctgcgttatttacgccagtgggtcaacgaatccggcattcgttggggcatagatgacgacaacgttcgcgagctggaactccccgccaccggacaacacacctggcgatttggcctgacgcgtatgttgttgggctacgcgatggagagcgcgcagggcgagtggcaatcggttctaccttatgatgaatcgagcggcttaattgcagaactggtggggcatctggcttcactgctaatgcagctaaacatctggcgtcgcgggctggcacaggagcgtccgctggaagagtggttgccggtttgtcgcgatatgctcaacgccttcttcctgccggatgcggaaaccgaagcggcgatgacgctgatcgaacaacaatggcaggcgattatcgccgaaggtttaggtgcgcagtatggcgacgcggtgccgctgtcactattgcgtgatgaactggcacagcgtctggatcaagaacgtatcagccagcgttttctcgccggaccggttaacatttgtactctgatgccaatgcgttcaattccgttcaaagtggtttgcctgctgggaatgaacgacggcgtttatccacgtcagcttgcgccattgggctttgacctgatgagccagaaaccgaagcgtggcgaccgtagccgtcgcgatgacgaccgctatctgttcctggaagcgttaatttccgcgcagcaaaaactctatatcagctatatcggtcgttccattcaggataacagtgagcgtttcccgtcggtactggtgcaggaactgatcgactacatcgggcaaagtcattatctaccgggcgatgaagcgctcaactgtgatgaaagcgaggcaagggtaaaagcgcatcttacttgcctccatacccggatgccgtttgatccacaaaactaccagccaggcgaacgacaaagctatgctcgtgaatggctacctgcggccagccaggctggtaaagcacattctgaatttgttcagccgctgccgtttaccttaccggaaaccgtgccgctggaaacgctacaacgattctgggcacatccggtgcgggcatttttccagatgcgtttgcaggtgaacttccgtactgaagacagcgaaatccccgacaccgagccatttattctggaaggacttagccgttatcaaatcaatcagcagttattgaatgcactggttgagcaggatgatgccgaacgcttgttccgccgcttccgagcggcaggggatttaccgtatggcgcttttggtgaaattttctgggaaacacagtgccaggagatgcagcagcttgccgacagagtcattgcctgtcgccagccggggcagagtatggaaattgatctcgcctgcaacggtgtgcagataactggctggttgccgcaggtgcagccggatggcctgttgcgctggcgtccctctttattaagtgtggcccagggaatgcaactttggctggaacaccttgtctactgtgccagcggtggtaatggtgaaagtcgcctttttctacgcaaagacggcgagtggcgttttccgccgcttgcagccgaacaggctttgcattacctctcacaactgattgaggggtatcgtgaaggaatgtccgcgccattgctggtgttacctgaaagtggcggcgcgtggctaaaaacctgttatgacgcgcaaaacgatgccatgctggatgacgattccacgttgcaaaaagcccgtacgaaattccttcaggcttacgaaggcaacatgatggtgcgtggcgaaggtgatgatatctggtatcaaaggctctggcggcaattaacaccagagacaatggaggccatcgttgaacagtcgcaacgtttcctgttaccgctgtttcgctttaatcagtca

SEQ ID NO: 41 - DNA, 1824 bp, Escherichia coli
  misc_feature (1)..(1824): Escherichia coli str. K-12 RecD (NC_000913.3)
atgaaattgcaaaagcaattactggaagctgtggagcacaaacagctacgcccgctggatgtgcaatttgccctgaccgtggcgggagatgaacatcctgccgtcaccctcgcggcggcactgttaagtcatgatgccggagagggacacgtttgtttgccgctttcacgactggaaaataacgaggcgtcgcatccgctgttggcgacctgtgtcagtgaaatcggtgagctacaaaattgggaagaatgcttgctggcttctcaagcggtcagcaggggagatgaacccacgccgatgatcctctgtggcgatcgtctttatttgaatcgcatgtggtgtaacgagcgcacagtggcacgctttttcaacgaagtgaatcatgccattgaggttgatgaagctctactggcgcaaaccctggacaaactttttccagtaagcgatgaaattaactggcaaaaagttgcggcggcagtggcgctgacgcggcggatctcggtgatttccggcggccctggcaccggtaaaacgaccaccgtagcgaagttgctggcagcgttaattcaaatggccgacggcgaacgctgccgtatccgtctggctgcaccaacgggtaaagctgccgcgcgcttaaccgaatctctcggcaaggctttgcgacagttaccgctgaccgatgaacaaaagaaacgcattccggaagatgccagcactttgcaccgattgctgggcgcgcagccgggtagccagcgtttacgtcatcatgccggtaacccgctgcatcttgatgtgctggtggtagatgaagcgtcaatgatcgatctgcctatgatgtcgagactgatcgacgccttgcccgatcatgcgcgagtgatctttctcggcgatcgtgatcaactggcctcggttgaggctggggctgtgctgggcgatatctgcgcttatgccaacgcgggctttaccgccgagcgtgccaggcagctaagccgcctgacggggactcacgttccggcaggaaccggcacagaagcggcatctttgcgcgacagtctctgcctgctgcaaaaaagctatcgtttcggcagcgattctggcattggtcagttagctgcggcgatcaaccgtggtgataaaacggcagtgaaaaccgtttttcagcaggattttactgatatcgaaaaacggcttttacagagcggcgaagattatattgcgatgcttgaggaagctcttgcgggttacggacgttatctggatctgctgcaagcgcgtgccgagccggatttaatcattcaggcgttcaatgagtaccagcttttgtgcgccctgcgggaagggccgtttggcgtggctggactgaatgagcgaattgagcagtttatgcaacagaagcgcaaaattcatcgtcatccgcactctcgttggtacgaaggtcgaccggtgatgattgcccgtaatgacagcgcgcttgggttgtttaatggcgatatcggtattgcgctggatcgcgggcaggggacgcgcgtctggtttgcgatgccggacggcaatattaagtctgtgcaaccgagtcgcttgccagagcacgaaactacgtgggcgatgacggtacataaatcgcagggatcggagttcgaccatgcggcgttgattttgccgagccaacgcacgccggtagtaacgcgagagctggtttataccgcggtgacccgcgcgcgtcgccgtctgtcgctgtatgccgatgagcgcatattaagtgcggcaatcgccactcgtactgagcggcgcagtggtctggcggcgttgtttagttcacgggaa

SEQ ID NO: 42 - DNA, 1065 bp, Homo sapiens
  misc_feature (1)..(1065): Homo sapiens HNRNPD (NM_031370)
atgtcggaggagcagttcggcggggacggggcggcggcagcggcaacggcggcggtaggcggctcggcgggcgagcaggagggagccatggtggcggcgacacagggggcagcggcggcggcgggaagcggagccgggaccgggggcggaaccgcgtctggaggcaccgaagggggcagcgccgagtcggagggggcgaagattgacgccagtaagaacgaggaggatgaaggccattcaaactcctccccacgacactctgaagcagcgacggcacagcgggaagaatggaaaatgtttataggaggccttagctgggacactacaaagaaagatctgaaggactacttttccaaatttggtgaagttgtagactgcactctgaagttagatcctatcacagggcgatcaaggggttttggctttgtgctatttaaagaatcggagagtgtagataaggtcatggatcaaaaagaacataaattgaatgggaaggtgattgatcctaaaagggccaaagccatgaaaacaaaagagccggttaaaaaaatttttgttggtggcctttctccagatacacctgaagagaaaataagggagtactttggtggttttggtgaggtggaatccatagagctccccatggacaacaagaccaataagaggcgtgggttctgctttattacctttaaggaagaagaaccagtgaagaagataatggaaaagaaataccacaatgttggtcttagtaaatgtgaaataaaagtagccatgtcgaaggaacaatatcagcaacagcaacagtggggatctagaggaggatttgcaggaagagctcgtggaagaggtggtggccccagtcaaaactggaaccagggatatagtaactattggaatcaaggctatggcaactatggatataacagccaaggttacggtggttatggaggatatgactacactggttacaacaactactatggatatggtgattatagcaaccagcagagtggttatgggaaggtatccaggcgaggtggtcatcaaaatagctacaaaccatac

SEQ ID NO: 43 - DNA, 2778 bp, Homo sapiens
  misc_feature (1)..(2778): Homo sapiens ETAA1 (NM_019002)
atgagtcggcgaaggaaacatgatgacagccctagcccgaagaaaacgccgcacaaaacagtggcggcggaggaatgcggctcggtggtcgagccagggaggaggcggctgagatcggcccgcggttcgtggccctgcggggctagagaggggcctcccgggccagtgcggcagcgagagcagcctccgaccgccgccctgtgcagtaaaagtaaccccgaggaaaggtatgaaacaccaaagagagcgctgaaaatggactcactgtcatcttccttcagttctcctaatgatccagatggacagaatgatatcttttgggatcagaattctccattgacaaagcagttaggtaaaggaagaaaaaaacagatttacaccacagatagtgatgagatttcacatattgttaatcgtattgctcctcaggatgaaaaaccaacaacaaattctatgctggacatgtggattggtgaaactgctattccttgtactcccagtgtagcaaaaggaaaatcaagagcaaaaatcagctgcacaaagttaaaaacacaaagtcaagaagaagaacttatgaaactggctaaacaatttgataaaaatatggaagagctagatgtgattcaagagcaaaacaagaggaattatgattttacccagatgatttcagaaacagagattttaagtaattataaagataatatacagatgtggtcattacataatatagttcccgaaatagataatgctacaaaaaagccaatcaaaggaaacaccaagatatctgtggcaaataatcaaaatagcagtcagaagccatttgaccaaattgctgaagcagcctttaatgctatttttgatggttctactcagaaatgtagcggacagttaagccaagaactgccagaggctttttggagcaccagtaatactacctttgtaaagacaaatgctttgaaagaggagaaaatcattactaatgaaactctggtcattgaaaaactgtcaaataaaaccccacgatcactttcttctcaagtagatacacccataatgacaaaatcatgtgtgacttcctgtactaaggagccagaaacttctaataagtacattgatgcatttactacaagtgattttgaggatgattgggaaaacttactaggtagtgaaccttttgctatgcaaaatatcgacatgcctgaactctttccttctaaaacagcccatgttactgatcaaaaggaaatttgtacctttaatagtaaaactgttaaaaatacgtcaagagcaaatacaagtccagatgccaggttaggagattcaaaagtattacaagatctttcttcaaagacatatgacagagaattaatagatgcagaatatagattttcaccaaattcaaataaatcaaacaaattatccactggaaataaaatgaaatttgagaactcttccaataaaattgttattcaagacgaaattcaaaattgtatagttacatctaatctgacaaaaataaaggaagatattcttactaactctactgaagcttctgaaaggaagtcagctttgaacacaaggtattctaatgaacagaaaaataagtgcattttaaatcagtctattaaagcccctgttaatactgatctttttggctctgcaaatctaggcagtaaaaccagtgttagtaacccaaatcagactagtgcatcaaaagtaggttctttctttgatgattggaatgatccctcatttgccaatgaaattattaaagcatgtcatcaattagataatacctgggaagcagatgatgtagatgatgatttgttgtaccaagcatgtgatgatattgaaagactaactcagcaacaagacattagaaaggacagtaagacatcagaaagtatatgtgagatcaataataattccgaacatggagccaaactaactcagcaacaagacattagaaaggacagtaagacatcagaaagtatatgtgagatcaataataattccgaacatggagccaaaaacatgtttgctatatctaaacaaggaagtaatttggtacaatcaaagcatttgaatccaggcagcatttcagtgcagacatctttgacaaatagctcacaaatagataagccaatgaagatggagaaaggggaaatgtatggaaattctccaagatttttaggtgccacaaatttgactatgtattctaagatctcaaactgtcagataaataatctgcatgtgtcttatactaacactgatgttccaatacaagtgaatagttccaaattggttcttccaggaagttcaagtttgaatgtaacttcagatcatatgaatacagaaattactacttataagaagaaattgagtactaatcagccatgccataagactgtaacagatgaagctcagagcaaccttaacacaacagttggattttcaaagtttacatttacaaggatgaaaaattctcagattctttctcagtttaatcaaaattgtataactggaagtatgtctgataccaaaattacacagggtgtggagaaaaagaaaggtgtcaacccattactggaggaagctgttggacagcaatctttggtgaaactttctgaatctttgaaacaatcttcaaaagaggaagaagagaaaaatagaaagtgttctcctgaagaaattcagagaaaaagacaagaagcactggttcggagaatggctaaagcacgagcctcatctgtaaatgcagctcccacttcatttctt

SEQ ID NO: 44 - DNA, 1179 bp, Homo sapiens
  misc_feature (1)..(1179): Homo sapiens DSCC1 (NM_024094)
atgaagaggacccgcgacgaggtggatgcgacgctgcagatcgccaagctgaatgcggccgagctgctgccggcggtgcactgcctgggcttcggccctggggccagcggcgctgcagccggcgacttctgcctgctggagctggagcccacgctgtgccagcagctggaggatggacacagtcttgtgattcgtggtgataaagacgagcaagctgtgctgtgcagtaaagacaaaacatacgacttgaagatagcagacacttccaatatgttgcttttcattcctggttgtaaaactccggaccagttgaagaaggaagattcacactgtaacattattcacactgagatctttggtttttctaataattattgggaattaagaagacgtagacccaagttaaagaagctaaagaaacttttgatggaaaatccatatgaaggacctgacagtcaaaaagagaaggattcaaatagctcaaaatatacaactgaagatttgcttgatcaaattcaggcaagtgaggaagaaataatgacccaattacaagttctaaatgcctgtaagattggaggttattggaggattcttgaatttgattatgagatgaaacttctgaatcatgtaactcagcttgtggattctgaatcatggtcttttggtaaagttcctttgaacacatgccttcaggaactcggaccattggagccagaggaaatgatagaacactgtcttaaatgttatgggaagaaatatgtagatgaaggcgaagtttattttgagttggatgctgataaaatatgtagagcagcagcacgaatgctacttcagaatgcggtgaaattcaatctcgctgagtttcaagaagtgtggcagcagagtgttcctgaaggaatggtaactagtcttgatcagcttaagggtttagcgctggtggatagacactcgagaccagaaatcatatttttgctgaaagtagatgatttacctgaggataatcaggaacgttttaatagccttttctctctaagggagaagtggacagaagaagatattgctccatatattcaagatttgtgtggagagaagcaaaccataggtgcattactcactaaatattctcattcttcgatgcaaaatggtgttaaagtttataattcgagaagacccatttct

SEQ ID NO: 45 - DNA, 3042 bp, Homo sapiens
  misc_feature (1)..(3042): Homo sapiens PARP1 (NM_001618)
atggcggagtcttcggataagctctatcgagtcgagtacgccaagagcgggcgcgcctcttgcaagaaatgcagcgagagcatccccaaggactcgctccggatggccatcatggtgcagtcgcccatgtttgatggaaaagtcccacactggtaccacttctcctgcttctggaaggtgggccactccatccggcaccctgacgttgaggtggatgggttctctgagcttcggtgggatgaccagcagaaagtcaagaagacagcggaagctggaggagtgacaggcaaaggccaggatggaattggtagcaaggcagagaagactctgggtgactttgcagcagagtatgccaagtccaacagaagtacgtgcaaggggtgtatggagaagatagaaaagggccaggtgcgcctgtccaagaagatggtggacccggagaagccacagctaggcatgattgaccgctggtaccatccaggctgctttgtcaagaacagggaggagctgggtttccggcccgagtacagtgcgagtcagctcaagggcttcagcctccttgctacagaggataaagaagccctgaagaagcagctcccaggagtcaagagtgaaggaaagagaaaaggcgatgaggtggatggagtggatgaagtggcgaagaagaaatctaaaaaagaaaaagacaaggatagtaagcttgaaaaagccctaaaggctcagaacgacctgatctggaacatcaaggacgagctaaagaaagtgtgttcaactaatgacctgaaggagctactcatcttcaacaagcagcaagtgccttctggggagtcggcgatcttggaccgagtagctgatggcatggtgttcggtgccctccttccctgcgaggaatgctcgggtcagctggtcttcaagagcgatgcctattactgcactggggacgtcactgcctggaccaagtgtatggtcaagacacagacacccaaccggaaggagtgggtaaccccaaaggaattccgagaaatctcttacctcaagaaattgaaggttaaaaaacaggaccgtatattccccccagaaaccagcgcctccgtggcggccacgcctccgccctccacagcctcggctcctgctgctgtgaactcctctgcttcagcagataagccattatccaacatgaagatcctgactctcgggaagctgtcccggaacaaggatgaagtgaaggccatgattgagaaactcggggggaagttgacggggacggccaacaaggcttccctgtgcatcagcaccaaaaaggaggtggaaaagatgaataagaagatggaggaagtaaaggaagccaacatccgagttgtgtctgaggacttcctccaggacgtctccgcctccaccaagagccttcaggagttgttcttagcgcacatcttgtccccttggggggcagaggtgaaggcagagcctgttgaagttgtggccccaagagggaagtcaggggctgcgctctccaaaaaaagcaagggccaggtcaaggaggaaggtatcaacaaatctgaaaagagaatgaaattaactcttaaaggaggagcagctgtggatcctgattctggactggaacactctgcgcatgtcctggagaaaggtgggaaggtcttcagtgccacccttggcctggtggacatcgttaaaggaaccaactcctactacaagctgcagcttctggaggacgacaaggaaaacaggtattggatattcaggtcctggggccgtgtgggtacggtgatcggtagcaacaaactggaacagatgccgtccaaggaggatgccattgagcacttcatgaaattatatgaagaaaaaaccgggaacgcttggcactccaaaaatttcacgaagtatcccaaaaagttctaccccctggagattgactatggccaggatgaagaggcagtgaagaagctgacagtaaatcctggcaccaagtccaagctccccaagccagttcaggacctcatcaagatgatctttgatgtggaaagtatgaagaaagccatggtggagtatgagatcgaccttcagaagatgcccttggggaagctgagcaaaaggcagatccaggccgcatactccatcctcagtgaggtccagcaggcggtgtctcagggcagcagcgactctcagatcctggatctctcaaatcgcttttacaccctgatcccccacgactttgggatgaagaagcctccgctcctgaacaatgcagacagtgtgcaggccaaggtggaaatgcttgacaacctgctggacatcgaggtggcctacagtctgctcaggggagggtctgatgatagcagcaaggatcccatcgatgtcaactatgagaagctcaaaactgacattaaggtggttgacagagattctgaagaagccgagatcatcaggaagtatgttaagaacactcatgcaaccacacacaatgcgtatgacttggaagtcatcgatatctttaagatagagcgtgaaggcgaatgccagcgttacaagccctttaagcagcttcataaccgaagattgctgtggcacgggtccaggaccaccaactttgctgggatcctgtcccagggtcttcggatagccccgcctgaagcgcccgtgacaggctacatgtttggtaaagggatctatttcgctgacatggtctccaagagtgccaactactgccatacgtctcagggagacccaataggcttaatcctgttgggagaagttgcccttggaaacatgtatgaactgaagcacgcttcacatatcagcaagttacccaagggcaagcacagtgtcaaaggtttgggcaaaactacccctgatccttcagctaacattagtctggatggtgtagacgttcctcttgggaccgggatttcatctggtgtgaatgacacctctctactatataacgagtacattgtctatgatattgctcaggtaaatctgaagtatctgctgaaactgaaattcaattttaagacctccctgtgg

SEQ ID NO: 46 - DNA, 5589 bp, Homo sapiens
  misc_feature (1)..(5589): Homo sapiens BRCA1 DNA repair associated (BRCA1, NM_007294)
atggatttatctgctcttcgcgttgaagaagtacaaaatgtcattaatgctatgcagaaaatcttagagtgtcccatctgtctggagttgatcaaggaacctgtctccacaaagtgtgaccacatattttgcaaattttgcatgctgaaacttctcaaccagaagaaagggccttcacagtgtcctttatgtaagaatgatataaccaaaaggagcctacaagaaagtacgagatttagtcaacttgttgaagagctattgaaaatcatttgtgcttttcagcttgacacaggtttggagtatgcaaacagctataattttgcaaaaaaggaaaataactctcctgaacatctaaaagatgaagtttctatcatccaaagtatgggctacagaaaccgtgccaaaagacttctacagagtgaacccgaaaatccttccttgcaggaaaccagtctcagtgtccaactctctaaccttggaactgtgagaactctgaggacaaagcagcggatacaacctcaaaagacgtctgtctacattgaattgggatctgattcttctgaagataccgttaataaggcaacttattgcagtgtgggagatcaagaattgttacaaatcacccctcaaggaaccagggatgaaatcagtttggattctgcaaaaaaggctgcttgtgaattttctgagacggatgtaacaaatactgaacatcatcaacccagtaataatgatttgaacaccactgagaagcgtgcagctgagaggcatccagaaaagtatcagggtagttctgtttcaaacttgcatgtggagccatgtggcacaaatactcatgccagctcattacagcatgagaacagcagtttattactcactaaagacagaatgaatgtagaaaaggctgaattctgtaataaaagcaaacagcctggcttagcaaggagccaacataacagatgggctggaagtaaggaaacatgtaatgataggcggactcccagcacagaaaaaaaggtagatctgaatgctgatcccctgtgtgagagaaaagaatggaataagcagaaactgccatgctcagagaatcctagagatactgaagatgttccttggataacactaaatagcagcattcagaaagttaatgagtggttttccagaagtgatgaactgttaggttctgatgactcacatgatggggagtctgaatcaaatgccaaagtagctgatgtattggacgttctaaatgaggtagatgaatattctggttcttcagagaaaatagacttactggccagtgatcctcatgaggctttaatatgtaaaagtgaaagagttcactccaaatcagtagagagtaatattgaagacaaaatatttgggaaaacctatcggaagaaggcaagcctccccaacttaagccatgtaactgaaaatctaattataggagcatttgttactgagccacagataatacaagagcgtcccctcacaaataaattaaagcgtaaaaggagacctacatcaggccttcatcctgaggattttatcaagaaagcagatttggcagttcaaaagactcctgaaatgataaatcagggaactaaccaaacggagcagaatggtcaagtgatgaatattactaatagtggtcatgagaataaaacaaaaggtgattctattcagaatgagaaaaatcctaacccaatagaatcactcgaaaaagaatctgctttcaaaacgaaagctgaacctataagcagcagtataagcaatatggaactcgaattaaatatccacaattcaaaagcacctaaaaagaataggctgaggaggaagtcttctaccaggcatattcatgcgcttgaactagtagtcagtagaaatctaagcccacctaattgtactgaattgcaaattgatagttgttctagcagtgaagagataaagaaaaaaaagtacaaccaaatgccagtcaggcacagcagaaacctacaactcatggaaggtaaagaacctgcaactggagccaagaagagtaacaagccaaatgaacagacaagtaaaagacatgacagcgatactttcccagagctgaagttaacaaatgcacctggttcttttactaagtgttcaaataccagtgaacttaaagaatttgtcaatcctagccttccaagagaagaaaaagaagagaaactagaaacagttaaagtgtctaataatgctgaagaccccaaagatctcatgttaagtggagaaagggttttgcaaactgaaagatctgtagagagtagcagtatttcattggtacctggtactgattatggcactcaggaaagtatctcgttactggaagttagcactctagggaaggcaaaaacagaaccaaataaatgtgtgagtcagtgtgcagcatttgaaaaccccaagggactaattcatggttgttccaaagataatagaaatgacacagaaggctttaagtatccattgggacatgaagttaaccacagtcgggaaacaagcatagaaatggaagaaagtgaacttgatgctcagtatttgcagaatacattcaaggtttcaaagcgccagtcatttgctccgttttcaaatccaggaaatgcagaagaggaatgtgcaacattctctgcccactctgggtccttaaagaaacaaagtccaaaagtcacttttgaatgtgaacaaaaggaagaaaatcaaggaaagaatgagtctaatatcaagcctgtacagacagttaatatcactgcaggctttcctgtggttggtcagaaagataagccagttgataatgccaaatgtagtatcaaaggaggctctaggttttgtctatcatctcagttcagaggcaacgaaactggactcattactccaaataaacatggacttttacaaaacccatatcgtataccaccactttttcccatcaagtcatttgttaaaactaaatgtaagaaaaatctgctagaggaaaactttgaggaacattcaatgtcacctgaaagagaaatgggaaatgagaacattccaagtacagtgagcacaattagccgtaataacattagagaaaatgtttttaaagaagccagctcaagcaatattaatgaagtaggttccagtactaatgaagtgggctccagtattaatgaaataggttccagtgatgaaaacattcaagcagaactaggtagaaacagagggccaaaattgaatgctatgcttagattaggggttttgcaacctgaggtctataaacaaagtcttcctggaagtaattgtaagcatcctgaaataaaaaagcaagaatatgaagaagtagttcagactgttaatacagatttctctccatatctgatttcagataacttagaacagcctatgggaagtagtcatgcatctcaggtttgttctgagacacctgatgacctgttagatgatggtgaaataaaggaagatactagttttgctgaaaatgacattaaggaaagttctgctgtttttagcaaaagcgtccagaaaggagagcttagcaggagtcctagccctttcacccatacacatttggctcagggttaccgaagaggggccaagaaattagagtcctcagaagagaacttatctagtgaggatgaagagcttccctgcttccaacacttgttatttggtaaagtaaacaatataccttctcagtctactaggcatagcaccgttgctaccgagtgtctgtctaagaacacagaggagaatttattatcattgaagaatagcttaaatgactgcagtaaccaggtaatattggcaaaggcatctcaggaacatcaccttagtgaggaaacaaaatgttctgctagcttgttttcttcacagtgcagtgaattggaagacttgactgcaaatacaaacacccaggatcctttcttgattggttcttccaaacaaatgaggcatcagtctgaaagccagggagttggtctgagtgacaaggaattggtttcagatgatgaagaaagaggaacgggcttggaagaaaataatcaagaagagcaaagcatggattcaaacttaggtgaagcagcatctgggtgtgagagtgaaacaagcgtctctgaagactgctcagggctatcctctcagagtgacattttaaccactcagcagagggataccatgcaacataacctgataaagctccagcaggaaatggctgaactagaagctgtgttagaacagcatgggagccagccttctaacagctacccttccatcataagtgactcttctgcccttgaggacctgcgaaatccagaacaaagcacatcagaaaaagcagtattaacttcacagaaaagtagtgaataccctataagccagaatccagaaggcctttctgctgacaagtttgaggtgtctgcagatagttctaccagtaaaaataaagaaccaggagtggaaaggtcatccccttctaaatgcccatcattagatgataggtggtacatgcacagttgctctgggagtcttcagaatagaaactacccatctcaagaggagctcattaaggttgttgatgtggaggagcaacagctggaagagtctgggccacacgatttgacggaaacatcttacttgccaaggcaagatctagagggaaccccttacctggaatctggaatcagcctcttctctgatgaccctgaatctgatccttctgaagacagagccccagagtcagctcgtgttggcaacataccatcttcaacctctgcattgaaagttccccaattgaaagttgcagaatctgcccagagtccagctgctgctcatactactgatactgctgggtataatgcaatggaagaaagtgtgagcagggagaagccagaattgacagcttcaacagaaagggtcaacaaaagaatgtccatggtggtgtctggcctgaccccagaagaatttatgctcgtgtacaagtttgccagaaaacaccacatcactttaactaatctaattactgaagagactactcatgttgttatgaaaacagatgctgagtttgtgtgtgaacggacactgaaatattttctaggaattgcgggaggaaaatgggtagttagctatttctgggtgacccagtctattaaagaaagaaaaatgctgaatgagcatgattttgaagtcagaggagatgtggtcaatggaagaaaccaccaaggtccaaagcgagcaagagaatcccaggacagaaagatcttcagggggctagaaatctgttgctatgggcccttcaccaacatgcccacagatcaactggaatggatggtacagctgtgtggtgcttctgtggtgaaggagctttcatcattcacccttggcacaggtgtccacccaattgtggttgtgcagccagatgcctggacagaggacaatggcttccatgcaattgggcagatgtgtgaggcacctgtggtgacccgagagtgggtgttggacagtgtagcactctaccagtgccaggagctggacacctacctgataccccagatcccccacagccactac

SEQ ID NO: 47 - DNA, 10254 bp, Homo sapiens
  misc_feature (1)..(10254): Homo sapiens BRCA2 (NM_000059)
atgcctattggatccaaagagaggccaacattttttgaaatttttaagacacgctgcaacaaagcagatttaggaccaataagtcttaattggtttgaagaactttcttcagaagctccaccctataattctgaacctgcagaagaatctgaacataaaaacaacaattacgaaccaaacctatttaaaactccacaaaggaaaccatcttataatcagctggcttcaactccaataatattcaaagagcaagggctgactctgccgctgtaccaatctcctgtaaaagaattagataaattcaaattagacttaggaaggaatgttcccaatagtagacataaaagtcttcgcacagtgaaaactaaaatggatcaagcagatgatgtttcctgtccacttctaaattcttgtcttagtgaaagtcctgttgttctacaatgtacacatgtaacaccacaaagagataagtcagtggtatgtgggagtttgtttcatacaccaaagtttgtgaagggtcgtcagacaccaaaacatatttctgaaagtctaggagctgaggtggatcctgatatgtcttggtcaagttctttagctacaccacccacccttagttctactgtgctcatagtcagaaatgaagaagcatctgaaactgtatttcctcatgatactactgctaatgtgaaaagctatttttccaatcatgatgaaagtctgaagaaaaatgatagatttatcgcttctgtgacagacagtgaaaacacaaatcaaagagaagctgcaagtcatggatttggaaaaacatcagggaattcatttaaagtaaatagctgcaaagaccacattggaaagtcaatgccaaatgtcctagaagatgaagtatatgaaacagttgtagatacctctgaagaagatagtttttcattatgtttttctaaatgtagaacaaaaaatctacaaaaagtaagaactagcaagactaggaaaaaaattttccatgaagcaaacgctgatgaatgtgaaaaatctaaaaaccaagtgaaagaaaaatactcatttgtatctgaagtggaaccaaatgatactgatccattagattcaaatgtagcaaatcagaagccctttgagagtggaagtgacaaaatctccaaggaagttgtaccgtctttggcctgtgaatggtctcaactaaccctttcaggtctaaatggagcccagatggagaaaatacccctattgcatatttcttcatgtgaccaaaatatttcagaaaaagacctattagacacagagaacaaaagaaagaaagattttcttacttcagagaattctttgccacgtatttctagcctaccaaaatcagagaagccattaaatgaggaaacagtggtaaataagagagatgaagagcagcatcttgaatctcatacagactgcattcttgcagtaaagcaggcaatatctggaacttctccagtggcttcttcatttcagggtatcaaaaagtctatattcagaataagagaatcacctaaagagactttcaatgcaagtttttcaggtcatatgactgatccaaactttaaaaaagaaactgaagcctctgaaagtggactggaaatacatactgtttgctcacagaaggaggactccttatgtccaaatttaattgataatggaagctggccagccaccaccacacagaattctgtagctttgaagaatgcaggtttaatatccactttgaaaaagaaaacaaataagtttatttatgctatacatgatgaaacatcttataaaggaaaaaaaataccgaaagaccaaaaatcagaactaattaactgttcagcccagtttgaagcaaatgcttttgaagcaccacttacatttgcaaatgctgattcaggtttattgcattcttctgtgaaaagaagctgttcacagaatgattctgaagaaccaactttgtccttaactagctcttttgggacaattctgaggaaatgttctagaaatgaaacatgttctaataatacagtaatctctcaggatcttgattataaagaagcaaaatgtaataaggaaaaactacagttatttattaccccagaagctgattctctgtcatgcctgcaggaaggacagtgtgaaaatgatccaaaaagcaaaaaagtttcagatataaaagaagaggtcttggctgcagcatgtcacccagtacaacattcaaaagtggaatacagtgatactgactttcaatcccagaaaagtcttttatatgatcatgaaaatgccagcactcttattttaactcctacttccaaggatgttctgtcaaacctagtcatgatttctagaggcaaagaatcatacaaaatgtcagacaagctcaaaggtaacaattatgaatctgatgttgaattaaccaaaaatattcccatggaaaagaatcaagatgtatgtgctttaaatgaaaattataaaaacgttgagctgttgccacctgaaaaatacatgagagtagcatcaccttcaagaaaggtacaattcaaccaaaacacaaatctaagagtaatccaaaaaaatcaagaagaaactacttcaatttcaaaaataactgtcaatccagactctgaagaacttttctcagacaatgagaataattttgtcttccaagtagctaatgaaaggaataatcttgctttaggaaatactaaggaacttcatgaaacagacttgacttgtgtaaacgaacccattttcaagaactctaccatggttttatatggagacacaggtgataaacaagcaacccaagtgtcaattaaaaaagatttggtttatgttcttgcagaggagaacaaaaatagtgtaaagcagcatataaaaatgactctaggtcaagatttaaaatcggacatctccttgaatatagataaaataccagaaaaaaataatgattacatgaacaaatgggcaggactcttaggtccaatttcaaatcacagttttggaggtagcttcagaacagcttcaaataaggaaatcaagctctctgaacataacattaagaagagcaaaatgttcttcaaagatattgaagaacaatatcctactagtttagcttgtgttgaaattgtaaataccttggcattagataatcaaaagaaactgagcaagcctcagtcaattaatactgtatctgcacatttacagagtagtgtagttgtttctgattgtaaaaatagtcatataacccctcagatgttattttccaagcaggattttaattcaaaccataatttaacacctagccaaaaggcagaaattacagaactttctactatattagaagaatcaggaagtcagtttgaatttactcagtttagaaaaccaagctacatattgcagaagagtacatttgaagtgcctgaaaaccagatgactatcttaaagaccacttctgaggaatgcagagatgctgatcttcatgtcataatgaatgccccatcgattggtcaggtagacagcagcaagcaatttgaaggtacagttgaaattaaacggaagtttgctggcctgttgaaaaatgactgtaacaaaagtgcttctggttatttaacagatgaaaatgaagtggggtttaggggcttttattctgctcatggcacaaaactgaatgtttctactgaagctctgcaaaaagctgtgaaactgtttagtgatattgagaatattagtgaggaaacttctgcagaggtacatccaataagtttatcttcaagtaaatgtcatgattctgttgtttcaatgtttaagatagaaaatcataatgataaaactgtaagtgaaaaaaataataaatgccaactgatattacaaaataatattgaaatgactactggcacttttgttgaagaaattactgaaaattacaagagaaatactgaaaatgaagataacaaatatactgctgccagtagaaattctcataacttagaatttgatggcagtgattcaagtaaaaatgatactgtttgtattcataaagatgaaacggacttgctatttactgatcagcacaacatatgtcttaaattatctggccagtttatgaaggagggaaacactcagattaaagaagatttgtcagatttaacttttttggaagttgcgaaagctcaagaagcatgtcatggtaatacttcaaataaagaacagttaactgctactaaaacggagcaaaatataaaagattttgagacttctgatacattttttcagactgcaagtgggaaaaatattagtgtcgccaaagagtcatttaataaaattgtaaatttctttgatcagaaaccagaagaattgcataacttttccttaaattctgaattacattctgacataagaaagaacaaaatggacattctaagttatgaggaaacagacatagttaaacacaaaatactgaaagaaagtgtcccagttggtactggaaatcaactagtgaccttccagggacaacccgaacgtgatgaaaagatcaaagaacctactctattgggttttcatacagctagcgggaaaaaagttaaaattgcaaaggaatctttggacaaagtgaaaaacctttttgatgaaaaagagcaaggtactagtgaaatcaccagttttagccatcaatgggcaaagaccctaaagtacagagaggcctgtaaagaccttgaattagcatgtgagaccattgagatcacagctgccccaaagtgtaaagaaatgcagaattctctcaataatgataaaaaccttgtttctattgagactgtggtgccacctaagctcttaagtgataatttatgtagacaaactgaaaatctcaaaacatcaaaaagtatctttttgaaagttaaagtacatgaaaatgtagaaaaagaaacagcaaaaagtcctgcaacttgttacacaaatcagtccccttattcagtcattgaaaattcagccttagctttttacacaagttgtagtagaaaaacttctgtgagtcagacttcattacttgaagcaaaaaaatggcttagagaaggaatatttgatggtcaaccagaaagaataaatactgcagattatgtaggaaattatttgtatgaaaataattcaaacagtactatagctgaaaatgacaaaaatcatctctccgaaaaacaagatacttatttaagtaacagtagcatgtctaacagctattcctaccattctgatgaggtatataatgattcaggatatctctcaaaaaataaacttgattctggtattgagccagtattgaagaatgttgaagatcaaaaaaacactagtttttccaaagtaatatccaatgtaaaagatgcaaatgcatacccacaaactgtaaatgaagatatttgcgttgaggaacttgtgactagctcttcaccctgcaaaaataaaaatgcagccattaaattgtccatatctaatagtaataattttgaggtagggccacctgcatttaggatagccagtggtaaaatcgtttgtgtttcacatgaaacaattaaaaaagtgaaagacatatttacagacagtttcagtaaagtaattaaggaaaacaacgagaataaatcaaaaatttgccaaacgaaaattatggcaggttgttacgaggcattggatgattcagaggatattcttcataactctctagataatgatgaatgtagcacgcattcacataaggtttttgctgacattcagagtgaagaaattttacaacataaccaaaatatgtctggattggagaaagtttctaaaatatcaccttgtgatgttagtttggaaacttcagatatatgtaaatgtagtatagggaagcttcataagtcagtctcatctgcaaatacttgtgggatttttagcacagcaagtggaaaatctgtccaggtatcagatgcttcattacaaaacgcaagacaagtgttttctgaaatagaagatagtaccaagcaagtcttttccaaagtattgtttaaaagtaacgaacattcagaccagctcacaagagaagaaaatactgctatacgtactccagaacatttaatatcccaaaaaggcttttcatataatgtggtaaattcatctgctttctctggatttagtacagcaagtggaaagcaagtttccattttagaaagttccttacacaaagttaagggagtgttagaggaatttgatttaatcagaactgagcatagtcttcactattcacctacgtctagacaaaatgtatcaaaaatacttcctcgtgttgataagagaaacccagagcactgtgtaaactcagaaatggaaaaaacctgcagtaaagaatttaaattatcaaataacttaaatgttgaaggtggttcttcagaaaataatcactctattaaagtttctccatatctctctcaatttcaacaagacaaacaacagttggtattaggaaccaaagtgtcacttgttgagaacattcatgttttgggaaaagaacaggcttcacctaaaaacgtaaaaatggaaattggtaaaactgaaactttttctgatgttcctgtgaaaacaaatatagaagtttgttctacttactccaaagattcagaaaactactttgaaacagaagcagtagaaattgctaaagcttttatggaagatgatgaactgacagattctaaactgccaagtcatgccacacattctctttttacatgtcccgaaaatgaggaaatggttttgtcaaattcaagaattggaaaaagaagaggagagccccttatcttagtgggagaaccctcaatcaaaagaaacttattaaatgaatttgacaggataatagaaaatcaagaaaaatccttaaaggcttcaaaaagcactccagatggcacaataaaagatcgaagattgtttatgcatcatgtttctttagagccgattacctgtgtaccctttcgcacaactaaggaacgtcaagagatacagaatccaaattttaccgcacctggtcaagaatttctgtctaaatctcatttgtatgaacatctgactttggaaaaatcttcaagcaatttagcagtttcaggacatccattttatcaagtttctgctacaagaaatgaaaaaatgagacacttgattactacaggcagaccaaccaaagtctttgttccaccttttaaaactaaatcacattttcacagagttgaacagtgtgttaggaatattaacttggaggaaaacagacaaaagcaaaacattgatggacatggctctgatgatagtaaaaataagattaatgacaatgagattcatcagtttaacaaaaacaactccaatcaagcagcagctgtaactttcacaaagtgtgaagaagaacctttagatttaattacaagtcttcagaatgccagagatatacaggatatgcgaattaagaagaaacaaaggcaacgcgtctttccacagccaggcagtctgtatcttgcaaaaacatccactctgcctcgaatctctctgaaagcagcagtaggaggccaagttccctctgcgtgttctcataaacagctgtatacgtatggcgtttctaaacattgcataaaaattaacagcaaaaatgcagagtcttttcagtttcacactgaagattattttggtaaggaaagtttatggactggaaaaggaatacagttggctgatggtggatggctcataccctccaatgatggaaaggctggaaaagaagaattttatagggctctgtgtgacactccaggtgtggatccaaagcttatttctagaatttgggtttataatcactatagatggatcatatggaaactggcagctatggaatgtgcctttcctaaggaatttgctaatagatgcctaagcccagaaagggtgcttcttcaactaaaatacagatatgatacggaaattgatagaagcagaagatcggctataaaaaagataatggaaagggatgacacagctgcaaaaacacttgttctctgtgtttctgacataatttcattgagcgcaaatatatctgaaacttctagcaataaaactagtagtgcagatacccaaaaagtggccattattgaacttacagatgggtggtatgctgttaaggcccagttagatcctcccctcttagctgtcttaaagaatggcagactgacagttggtcagaagattattcttcatggagcagaactggtgggctctcctgatgcctgtacacctcttgaagccccagaatctcttatgttaaagatttctgctaacagtactcggcctgctcgctggtataccaaacttggattctttcctgaccctagaccttttcctctgcccttatcatcgcttttcagtgatggaggaaatgttggttgtgttgatgtaattattcaaagagcataccctatacagtggatggagaagacatcatctggattatacatatttcgcaatgaaagagaggaagaaaaggaagcagcaaaatatgtggaggcccaacaaaagagactagaagccttattcactaaaattcaggaggaatttgaagaacatgaagaaaacacaacaaaaccatatttaccatcacgtgcactaacaagacagcaagttcgtgctttgcaagatggtgcagagctttatgaagcagtgaagaatgcagcagacccagcttaccttgagggttatttcagtgaagagcagttaagagccttgaataatcacaggcaaatgttgaatgataagaaacaagctcagatccagttggaaattaggaaggccatggaatctgctgaacaaaaggaacaaggtttatcaagggatgtcacaaccgtgtggaagttgcgtattgtaagctattcaaaaaaagaaaaagattcagttatactgagtatttggcgtccatcatcagatttatattctctgttaacagaaggaaagagatacagaatttatcatcttgcaacttcaaaatctaaaagtaaatctgaaagagctaacatacagttagcagcgacaaaaaaaactcagtatcaacaactaccggtttcagatgaaattttatttcagatttaccagccacgggagccccttcacttcagcaaatttttagatccagactttcagccatcttgttctgaggtggacctaataggatttgtcgtttctgttgtgaaaaaaacaggacttgcccctttcgtctatttgtcagacgaatgttacaatttactggcaataaagttttggatagaccttaatgaggacattattaagcctcatatgttaattgctgcaagcaacctccagtggcgaccagaatccaaatcaggccttcttactttatttgctggagatttttctgtgttttctgctagtccaaaagagggccactttcaagagacattcaacaaaatgaaaaatactgttgagaatattgacatactttgcaatgaagcagaaaacaagcttatgcatatactgcatgcaaatgatcccaagtggtccaccccaactaaagactgtacttcagggccgtacactgctcaaatcattcctggtacaggaaacaagcttctgatgtcttctcctaattgtgagatatattatcaaagtcctttatcactttgtatggccaaaaggaagtctgtttccacacctgtctcagcccagatgacttcaaagtcttgtaaaggggagaaagagattgatgaccaaaagaactgcaaaaagagaagagccttggatttcttgagtagactgcctttacctccacctgttagtcccatttgtacatttgtttctccggctgcacagaaggcatttcagccaccaaggagttgtggcaccaaatacgaaacacccataaagaaaaaagaactgaattctcctcagatgactccatttaaaaaattcaatgaaatttctcttttggaaagtaattcaatagctgacgaagaacttgcattgataaatacccaagctcttttgtctggttcaacaggagaaaaacaatttatatctgtcagtgaatccactaggactgctcccaccagttcagaagattatctcagactgaaacgacgttgtactacatctctgatcaaagaacaggagagttcccaggccagtacggaagaatgtgagaaaaataagcaggacacaattacaactaaaaaatatatc

SEQ ID NO: 48 - DNA, 1848 bp, Homo sapiens
  misc_feature (1)..(1848): Homo sapiens RPA1 (NM_002945)
atggtcggccaactgagcgagggggccattgcggccatcatgcagaagggggatacaaacataaagcccatcctccaagtcatcaacatccgtcccattactacggggaatagtccgccgcgttatcgactgctcatgagtgatggattgaacactctatcctctttcatgttggcgacacagttgaaccctctcgtggaggaagaacaattgtccagcaactgtgtatgccagattcacagatttattgtgaacactctgaaagacggaaggagagtagttatcttgatggaattagaagttttgaagtcagctgaagcagttggagtgaagattggcaatccagtgccctataatgaaggactcgggcagccgcaagtagctcctccagcgccagcagccagcccagcagcaagcagcaggccccagccgcagaatggaagctcgggaatgggttctactgtttctaaggcttatggtgcttcaaagacatttggaaaagctgcaggtcccagcctgtcacacacttctgggggaacacagtccaaagtggtgcccattgccagcctcactccttaccagtccaagtggaccatttgtgctcgtgttaccaacaaaagtcagatccgtacctggagcaactcccgaggggaagggaagcttttctccctagaactggttgacgaaagtggtgaaatccgagctacagctttcaatgagcaagtggacaagttctttcctcttattgaagtgaacaaggtgtattatttctcgaaaggcaccctgaagattgctaacaagcagttcacagctgttaaaaatgactacgagatgaccttcaataacgagacttccgtcatgccctgtgaggacgaccatcatttacctacggttcagtttgatttcacggggattgatgacctcgagaacaagtcgaaagactcacttgtagacatcatcgggatctgcaagagctatgaagacgccactaaaatcacagtgaggtctaacaacagagaagttgccaagaggaatatctacttgatggacacatccgggaaggtggtgactgctacactgtggggggaagatgctgataaatttgatggttctagacagcccgtgttggctatcaaaggagcccgagtctctgatttcggtggacggagcctctccgtgctgtcttcaagcactatcattgcgaatcctgacatcccagaggcctataagcttcgtggatggtttgacgcagaaggacaagccttagatggtgtttccatctctgatctaaagagcggcggagtcggagggagtaacaccaactggaaaaccttgtatgaggtcaaatccgagaacctgggccaaggcgacaagccggactactttagttctgtggccacagtggtgtatcttcgcaaagagaactgcatgtaccaagcctgcccgactcaggactgcaataagaaagtgattgatcaacagaatggattgtaccgctgtgagaagtgcgacaccgaatttcccaatttcaagtaccgcatgatcctgtcagtaaatattgcagattttcaagagaatcagtgggtgacttgtttccaggagtctgctgaagctatccttggacaaaatgctgcttatcttggggaattaaaagacaagaatgaacaggcatttgaagaagttttccagaatgccaacttccgatctttcatattcagagtcagggtcaaagtggagacctacaacgacgagtctcgaattaaggccactgtgatggacgtgaagcccgtggactacagagagtatggccgaaggctggtcatgagcatcaggagaagtgcattgatg

SEQ ID NO: 49 - DNA, 810 bp, Homo sapiens
  misc_feature (1)..(810): Homo sapiens RPA2 (NM_002946)
atgtggaacagtggattcgaaagctatggcagctcctcatacgggggagccggcggctacacgcagtccccggggggctttggatcgcccgcaccttctcaagccgaaaagaaatcaagagcccgagcccagcacattgtgccctgtactatatctcagctgctttctgccactttggttgatgaagtgttcagaattgggaatgttgagatttcacaggtcactattgtggggatcatcagacatgcagagaaggctccaaccaacattgtttacaaaatagatgacatgacagctgcacccatggacgttcgccagtgggttgacacagatgacaccagcagtgaaaacactgtggttcctccagaaacatatgtgaaagtggcaggccacctgagatcttttcagaacaaaaagagcctggtagcctttaagatcatgcccctggaggatatgaatgagttcaccacacatattctggaagtgatcaatgcacacatggtactaagcaaagccaacagccagccctcagcagggagagcacctatcagcaatccaggaatgagtgaagcagggaactttggtgggaatagcttcatgccagcaaatggcctcactgtggcccaaaaccaggtgttgaatttgattaaggcttgtccaagacctgaagggttgaactttcaggatctcaagaaccagctgaaacacatgtctgtatcctcaatcaagcaagctgtggattttctgagcaatgaggggcacatctattctactgtggatgatgaccattttaaatccacagatgcagaa

SEQ ID NO: 50 - DNA, 1017 bp, Homo sapiens
  misc_feature (1)..(1017): Homo sapiens RAD51 (NM_002875)
atggcaatgcagatgcagcttgaagcaaatgcagatacttcagtggaagaagaaagctttggcccacaacccatttcacggttagagcagtgtggcataaatgccaacgatgtgaagaaattggaagaagctggattccatactgtggaggctgttgcctatgcgccaaagaaggagctaataaatattaagggaattagtgaagccaaagctgataaaattctggctgaggcagctaaattagttccaatgggtttcaccactgcaactgaattccaccaaaggcggtcagagatcatacagattactactggctccaaagagcttgacaaactacttcaaggtggaattgagactggatctatcacagaaatgtttggagaattccgaactgggaagacccagatctgtcatacgctagctgtcacctgccagcttcccattgaccggggtggaggtgaaggaaaggccatgtacattgacactgagggtacctttaggccagaacggctgctggcagtggctgagaggtatggtctctctggcagtgatgtcctggataatgtagcatatgctcgagcgttcaacacagaccaccagacccagctcctttatcaagcatcagccatgatggtagaatctaggtatgcactgcttattgtagacagtgccaccgccctttacagaacagactactcgggtcgaggtgagctttcagccaggcagatgcacttggccaggtttctgcggatgcttctgcgactcgctgatgagtttggtgtagcagtggtaatcactaatcaggtggtagctcaagtggatggagcagcgatgtttgctgctgatcccaaaaaacctattggaggaaatatcatcgcccatgcatcaacaaccagattgtatctgaggaaaggaagaggggaaaccagaatctgcaaaatctacgactctccctgtcttcctgaagctgaagctatgttcgccattaatgcagatggagtgggagatgccaaagac

SEQ ID NO: 51 - DNA, 891 bp, Homo sapiens
  misc_feature (1)..(891): Homo sapiens CDK1 (NM_001786)
atggaagattataccaaaatagagaaaattggagaaggtacctatggagttgtgtataagggtagacacaaaactacaggtcaagtggtagccatgaaaaaaatcagactagaaagtgaagaggaaggggttcctagtactgcaattcgggaaatttctctattaaaggaacttcgtcatccaaatatagtcagtcttcaggatgtgcttatgcaggattccaggttatatctcatctttgagtttctttccatggatctgaagaaatacttggattctatccctcctggtcagtacatggattcttcacttgttaagagttatttataccaaatcctacaggggattgtgttttgtcactctagaagagttcttcacagagacttaaaacctcaaaatctcttgattgatgacaaaggaacaattaaactggctgattttggccttgccagagcttttggaatacctatcagagtatatacacatgaggtagtaacactctggtacagatctccagaagtattgctggggtcagctcgttactcaactccagttgacatttggagtataggcaccatatttgctgaactagcaactaagaaaccacttttccatggggattcagaaattgatcaactcttcaggattttcagagctttgggcactcccaataatgaagtgtggccagaagtggaatctttacaggactataagaatacatttcccaaatggaaaccaggaagcctagcatcccatgtcaaaaacttggatgaaaatggcttggatttgctctcgaaaatgttaatctatgatccagccaaacgaatttctggcaaaatggcactgaatcatccatattttaatgatttggacaatcagattaagaagatg

SEQ ID NO: 52 - DNA, 894 bp, Homo sapiens
  misc_feature (1)..(894): Homo sapiens CDK2 (NM_001798)
atggagaacttccaaaaggtggaaaagatcggagagggcacgtacggagttgtgtacaaagccagaaacaagttgacgggagaggtggtggcgcttaagaaaatccgcctggacactgagactgagggtgtgcccagtactgccatccgagagatctctctgcttaaggagcttaaccatcctaatattgtcaagctgctggatgtcattcacacagaaaataaactctacctggtttttgaatttctgcaccaagatctcaagaaattcatggatgcctctgctctcactggcattcctcttcccctcatcaagagctatctgttccagctgctccagggcctagctttctgccattctcatcgggtcctccaccgagaccttaaacctcagaatctgcttattaacacagagggggccatcaagctagcagactttggactagccagagcttttggagtccctgttcgtacttacacccatgaggtggtgaccctgtggtaccgagctcctgaaatcctcctgggctgcaaatattattccacagctgtggacatctggagcctgggctgcatctttgctgagatggtgactcgccgggccctattccctggagattctgagattgaccagctcttccggatctttcggactctggggaccccagatgaggtggtgtggccaggagttacttctatgcctgattacaagccaagtttccccaagtgggcccggcaagattttagtaaagttgtacctcccctggatgaagatggacggagcttgttatcgcaaatgctgcactacgaccctaacaagcggatttcggccaaggcagccctggctcaccctttcttccaggatgtgaccaagccagtaccccatcttcgactc

SEQ ID NO: 53 - DNA, 2691 bp, Homo sapiens
  misc_feature (1)..(2691): Homo sapiens RBBP8, CtIP (NM_002894)
atgaacatctcgggaagcagctgtggaagccctaactctgcagatacatctagtgactttaaggacctttggacaaaactaaaagaatgtcatgatagagaagtacaaggtttacaagtaaaagtaaccaagctaaaacaggaacgaatcttagatgcacaaagactagaagaattcttcaccaaaaatcaacagctgagggaacagcagaaagtccttcatgaaaccattaaagttttagaagatcggttaagagcaggcttatgtgatcgctgtgcagtaactgaagaacatatgcggaaaaaacagcaagagtttgaaaatatccggcagcagaatcttaaacttattacagaacttatgaatgaaaggaatactctacaggaagaaaataaaaagctttctgaacaactccagcagaaaattgagaatgatcaacagcatcaagcagctgagcttgaatgtgaggaagacgttattccagattcaccgataacagccttctcattttctggcgttaaccggctacgaagaaaggagaacccccatgtccgatacatagaacaaacacatactaaattggagcactctgtgtgtgcaaatgaaatgagaaaagtttccaagtcttcaactcatccacaacataatcctaatgaaaatgaaattctagtagctgacacttatgaccaaagtcaatctccaatggccaaagcacatggaacaagcagctatacccctgataagtcatcttttaatttagctacagttgttgctgaaacacttggacttggtgttcaagaagaatctgaaactcaaggtcccatgagcccccttggtgatgagctctaccactgtctggaaggaaatcacaagaaacagccttttgaggaatctacaagaaatactgaagatagtttaagattttcagattctacttcaaagactcctcctcaagaagaattacctactcgagtgtcatctcctgtatttggagctacctctagtatcaaaagtggtttagatttgaatacaagtttgtccccttctcttttacagcctgggaaaaaaaaacatctgaaaacactcccttttagcaacacttgtatatctagattagaaaaaactagatcaaaatctgaagatagtgcccttttcacacatcacagtcttgggtctgaagtgaacaagatcattatccagtcatctaataaacagatacttataaataaaaatataagtgaatccctaggtgaacagaataggactgagtacggtaaagattctaacactgataaacatttggagcccctgaaatcattgggaggccgaacatccaaaaggaagaaaactgaggaagaaagtgaacatgaagtaagctgcccccaagcttcttttgataaagaaaatgctttcccttttccaatggataatcagttttccatgaatggagactgtgtgatggataaacctctggatctgtctgatcgattttcagctattcagcgtcaagagaaaagccaaggaagtgagacttctaaaaacaaatttaggcaagtgactctttatgaggctttgaagaccattccaaagggcttttcctcaagccgtaaggcctcagatggcaactgcacgttgcccaaagattccccaggggagccctgttcacaggaatgcatcatccttcagcccttgaataaatgctctccagacaataaaccatcattacaaataaaagaagaaaatgctgtctttaaaattcctctacgtccacgtgaaagtttggagactgagaatgttttagatgacataaagagtgctggttctcatgagccaataaaaatacaaaccaggtcagaccatggaggatgtgaacttgcatcagttcttcagttaaatccatgtagaactggtaaaataaagtctctacaaaacaaccaagatgtatcctttgaaaatatccagtggagtatagatccgggagcagacctttctcagtataaaatggatgttactgtaatagatacaaaggatggcagtcagtcaaaattaggaggagagacagtggacatggactgtacattggttagtgaaaccgttctcttaaaaatgaagaagcaagagcagaagggagaaaaaagttcaaatgaagaaagaaaaatgaatgatagcttggaagatatgtttgatcggacaacacatgaagagtatgaatcctgtttggcagacagtttctcccaagcagcagatgaagaggaggaattgtctactgccacaaagaaactacacactcatggtgataaacaagacaaagtcaagcagaaagcgtttgtggagccgtattttaaaggtgatgaaagagagactagcttgcaaaattttcctcatattgaggtggttcggaaaaaagaggagagaagaaaactgcttgggcacacgtgtaaggaatgtgaaatttattatgcagatatgccagcagaagaaagagaaaagaaattggcttcctgctcaagacaccgattccgctacattccacccaacacaccagagaatttttgggaagttggttttccttccactcagacttgtatggaaagaggttatattaaggaagatcttgatccttgtcctcgtccaaaaagacgtcagccttacaacgcaatattttctccaaaaggcaaggagcagaagaca

SEQ ID NO: 54 - DNA, 9168 bp, Homo sapiens
  misc_feature (1)..(9168): Homo sapiens ATM (NM_001351834)
atgagtctagtacttaatgatctgcttatctgctgccgtcaactagaacatgatagagctacagaacgaaagaaagaagttgagaaatttaagcgcctgattcgagatcctgaaacaattaaacatctagatcggcattcagattccaaacaaggaaaatatttgaattgggatgctgtttttagatttttacagaaatatattcagaaagaaacagaatgtctgagaatagcaaaaccaaatgtatcagcctcaacacaagcctccaggcagaaaaagatgcaggaaatcagtagtttggtcaaatacttcatcaaatgtgcaaacagaagagcacctaggctaaaatgtcaagaactcttaaattatatcatggatacagtgaaagattcatctaatggtgctatttacggagctgattgtagcaacatactactcaaagacattctttctgtgagaaaatactggtgtgaaatatctcagcaacagtggttagaattgttctctgtgtacttcaggctctatctgaaaccttcacaagatgttcatagagttttagtggctagaataattcatgctgttaccaaaggatgctgttctcagactgacggattaaattccaaatttttggactttttttccaaggctattcagtgtgcgagacaagaaaagagctcttcaggtctaaatcatatcttagcagctcttactatcttcctcaagactttggctgtcaactttcgaattcgagtgtgtgaattaggagatgaaattcttcccactttgctttatatttggactcaacataggcttaatgattctttaaaagaagtcattattgaattatttcaactgcaaatttatatccatcatccgaaaggagccaaaacccaagaaaaaggtgcttatgaatcaacaaaatggagaagtattttatacaacttatatgatctgctagtgaatgagataagtcatataggaagtagaggaaagtattcttcaggatttcgtaatattgccgtcaaagaaaatttgattgaattgatggcagatatctgtcaccaggtttttaatgaagataccagatccttggagatttctcaatcttacactactacacaaagagaatctagtgattacagtgtcccttgcaaaaggaagaaaatagaactaggctgggaagtaataaaagatcaccttcagaagtcacagaatgattttgatcttgtgccttggctacagattgcaacccaattaatatcaaagtatcctgcaagtttacctaactgtgagctgtctccattactgatgatactatctcagcttctaccccaacagcgacatggggaacgtacaccatatgtgttacgatgccttacggaagttgcattgtgtcaagacaagaggtcaaacctagaaagctcacaaaagtcagatttattaaaactctggaataaaatttggtgtattacctttcgtggtataagttctgagcaaatacaagctgaaaactttggcttacttggagccataattcagggtagtttagttgaggttgacagagaattctggaagttatttactgggtcagcctgcagaccttcatgtcctgcagtatgctgtttgactttggcactgaccaccagtatagttccaggaacggtaaaaatgggaatagagcaaaatatgtgtgaagtaaatagaagcttttctttaaaggaatcaataatgaaatggctcttattctatcagttagagggtgacttagaaaatagcacagaagtgcctccaattcttcacagtaattttcctcatcttgtactggagaaaattcttgtgagtctcactatgaaaaactgtaaagctgcaatgaattttttccaaagcgtgccagaatgtgaacaccaccaaaaagataaagaagaactttcattctcagaagtagaagaactatttcttcagacaacttttgacaagatggactttttaaccattgtgagagaatgtggtatagaaaagcaccagtccagtattggcttctctgtccaccagaatctcaaggaatcactggatcgctgtcttctgggattatcagaacagcttctgaataattactcatctgagattacaaattcagaaactcttgtccggtgttcacgtcttttggtgggtgtccttggctgctactgttacatgggtgtaatagctgaagaggaagcatataagtcagaattattccagaaagccaagtctctaatgcaatgtgcaggagaaagtatcactctgtttaaaaataagacaaatgaggaattcagaattggttccttgagaaatatgatgcagctatgtacacgttgcttgagcaactgtaccaagaagagtccaaataagattgcatctggctttttcctgcgattgttaacatcaaagctaatgaatgacattgcagatatttgtaaaagtttagcatccttcatcaaaaagccatttgaccgtggagaagtagaatcaatggaagatgatactaatggaaatctaatggaggtggaggatcagtcatccatgaatctatttaacgattaccctgatagtagtgttagtgatgcaaacgaacctggagagagccaaagtaccataggtgccattaatcctttagctgaagaatatctgtcaaagcaagatctacttttcttagacatgctcaagttcttgtgtttgtgtgtaactactgctcagaccaatactgtgtcctttagggcagctgatattcggaggaaattgttaatgttaattgattctagcacgctagaacctaccaaatccctccacctgcatatgtatctaatgcttttaaaggagcttcctggagaagagtaccccttgccaatggaagatgttcttgaacttctgaaaccactatccaatgtgtgttctttgtatcgtcgtgaccaagatgtttgtaaaactattttaaaccatgtccttcatgtagtgaaaaacctaggtcaaagcaatatggactctgagaacacaagggatgctcaaggacagtttcttacagtaattggagcattttggcatctaacaaaggagaggaaatatatattctctgtaagaatggccctagtaaattgccttaaaactttgcttgaggctgatccttattcaaaatgggccattcttaatgtaatgggaaaagactttcctgtaaatgaagtatttacacaatttcttgctgacaatcatcaccaagttcgcatgttggctgcagagtcaatcaatagattgttccaggacacgaagggagattcttccaggttactgaaagcacttcctttgaagcttcagcaaacagcttttgaaaatgcatacttgaaagctcaggaaggaatgagagaaatgtcccatagtgctgagaaccctgaaactttggatgaaatttataatagaaaatctgttttactgacgttgatagctgtggttttatcctgtagccctatctgcgaaaaacaggctttgtttgccctgtgtaaatctgtgaaagagaatggattagaacctcaccttgtgaaaaaggttttagagaaagtttctgaaacttttggatatagacgtttagaagactttatggcatctcatttagattatctggttttggaatggctaaatcttcaagatactgaatacaacttatcttcttttccttttattttattaaactacacaaatattgaggatttctatagatcttgttataaggttttgattccacatctggtgattagaagtcattttgatgaggtgaagtccattgctaatcagattcaagaggactggaaaagtcttctaacagactgctttccaaagattcttgtaaatattcttccttattttgcctatgagggtaccagagacagtgggatggcacagcaaagagagactgctaccaaggtctatgatatgcttaaaagtgaaaacttattgggaaaacagattgatcacttattcattagtaatttaccagagattgtggtggagttattgatgacgttacatgagccagcaaattctagtgccagtcagagcactgacctctgtgacttttcaggggatttggatcctgctcctaatccacctcattttccatcgcatgtgattaaagcaacatttgcctatatcagcaattgtcataaaaccaagttaaaaagcattttagaaattctttccaaaagccctgattcctatcagaaaattcttcttgccatatgtgagcaagcagctgaaacaaataatgtttataagaagcacagaattcttaaaatatatcacctgtttgttagtttattactgaaagatataaaaagtggcttaggaggagcttgggcctttgttcttcgagacgttatttatactttgattcactatatcaaccaaaggccttcttgtatcatggatgtgtcattacgtagcttctccctttgttgtgacttattaagtcaggtttgccagacagccgtgacttactgtaaggatgctctagaaaaccatcttcatgttattgttggtacacttataccccttgtgtatgagcaggtggaggttcagaaacaggtattggacttgttgaaatacttagtgatagataacaaggataatgaaaacctctatatcacgattaagcttttagatccttttcctgaccatgttgtttttaaggatttgcgtattactcagcaaaaaatcaaatacagtagaggacccttttcactcttggaggaaattaaccattttctctcagtaagtgtttatgatgcacttccattgacaagacttgaaggactaaaggatcttcgaagacaactggaactacataaagatcagatggtggacattatgagagcttctcaggataatccgcaagatgggattatggtgaaactagttgtcaatttgttgcagttatccaagatggcaataaaccacactggtgaaaaagaagttctagaggctgttggaagctgcttgggagaagtgggtcctatagatttctctaccatagctatacaacatagtaaagatgcatcttataccaaggcccttaagttatttgaagataaagaacttcagtggaccttcataatgctgacctacctgaataacacactggtagaagattgtgtcaaagttcgatcagcagctgttacctgtttgaaaaacattttagccacaaagactggacatagtttctgggagatttataagatgacaacagatccaatgctggcctatctacagccttttagaacatcaagaaaaaagtttttagaagtacccagatttgacaaagaaaacccttttgaaggcctggatgatataaatctgtggattcctctaagtgaaaatcatgacatttggataaagacactgacttgtgcttttttggacagtggaggcacaaaatgtgaaattcttcaattattaaagccaatgtgtgaagtgaaaactgacttttgtcagactgtacttccatacttgattcatgatattttactccaagatacaaatgaatcatggagaaatctgctttctacacatgttcagggatttttcaccagctgtcttcgacacttctcgcaaacgagccgatccacaacccctgcaaacttggattcagagtcagagcactttttccgatgctgtttggataaaaaatcacaaagaacaatgcttgctgttgtggactacatgagaagacaaaagagaccttcttcaggaacaatttttaatgatgctttctggctggatttaaattatctagaagttgccaaggtagctcagtcttgtgctgctcactttacagctttactctatgcagaaatctatgcagataagaaaagtatggatgatcaagagaaaagaagtcttgcatttgaagaaggaagccagagtacaactatttctagcttgagtgaaaaaagtaaagaagaaactggaataagtttacaggatcttctcttagaaatctacagaagtataggggagccagatagtttgtatggctgtggtggagggaagatgttacaacccattactagactacgaacatatgaacacgaagcaatgtggggcaaagccctagtaacatatgacctcgaaacagcaatcccctcatcaacacgccaggcaggaatcattcaggccttgcagaatttgggactctgccatattctttccgtctatttaaaaggattggattatgaaaataaagactggtgtcctgaactagaagaacttcattaccaagcagcatggaggaatatgcagtgggaccattgcacttccgtcagcaaagaagtagaaggaaccagttaccatgaatcattgtacaatgctctacaatctctaagagacagagaattctctacattttatgaaagtctcaaatatgccagagtaaaagaagtggaagagatgtgtaagcgcagccttgagtctgtgtattcgctctatcccacacttagcaggttgcaggccattggagagctggaaagcattggggagcttttctcaagatcagtcacacatagacaactctctgaagtatatattaagtggcagaaacactcccagcttctcaaggacagtgattttagttttcaggagcctatcatggctctacgcacagtcattttggagatcctgatggaaaaggaaatggacaactcacaaagagaatgtattaaggacattctcaccaaacaccttgtagaactctctatactggccagaactttcaagaacactcagctccctgaaagggcaatatttcaaattaaacagtacaattcagttagctgtggagtctctgagtggcagctggaagaagcacaagtattctgggcaaaaaaggagcagagtcttgccctgagtattctcaagcaaatgatcaagaagttggatgccagctgtgcagcgaacaatcccagcctaaaacttacatacacagaatgtctgagggtttgtggcaactggttagcagaaacgtgcttagaaaatcctgcggtcatcatgcagacctatctagaaaaggcagtagaagttgctggaaattatgatggagaaagtagtgatgagctaagaaatggaaaaatgaaggcatttctctcattagcccggttttcagatactcaataccaaagaattgaaaactacatgaaatcatcggaatttgaaaacaagcaagctctcctgaaaagagccaaagaggaagtaggtctccttagggaacataaaattcagacaaacagatacacagtaaaggttcagcgagagctggagttggatgaattagccctgcgtgcactgaaagaggatcgtaaacgcttcttatgtaaagcagttgaaaattatatcaactgcttattaagtggagaagaacatgatatgtgggtattccgactttgttccctctggcttgaaaattctggagtttctgaagtcaatggcatgatgaagagagacggaatgaagattccaacatataaatttttgcctcttatgtaccaattggctgctagaatggggaccaagatgatgggaggcctaggatttcatgaagtcctcaataatctaatctctagaatttcaatggatcacccccatcacactttgtttattatactggccttagcaaatgcaaacagagatgaatttctgactaaaccagaggtagccagaagaagcagaataactaaaaatgtgcctaaacaaagctctcagcttgatgaggatcgaacagaggctgcaaatagaataatatgtactatcagaagtaggagacctcagatggtcagaagtgttgaggcactttgtgatgcttatattatattagcaaacttagatgccactcagtggaagactcagagaaaaggcataaatattccagcagaccagccaattactaaacttaagaatttagaagatgttgttgtccctactatggaaattaaggtggaccacacaggagaatatggaaatctggtgactatacagtcatttaaagcagaatttcgcttagcaggaggtgtaaatttaccaaaaataatagattgtgtaggttccgatggcaaggagaggagacagcttgttaagggccgtgatgacctgagacaagatgctgtcatgcaacaggtcttccagatgtgtaatacattactgcagagaaacacggaaactaggaagaggaaattaactatctgtacttataaggtggttcccctctctcagcgaagtggtgttcttgaatggtgcacaggaactgtccccattggtgaatttcttgttaacaatgaagatggtgctcataaaagatacaggccaaatgatttcagtgcctttcagtgccaaaagaaaatgatggaggtgcaaaaaaagtcttttgaagagaaatatgaagtcttcatggatgtttgccaaaattttcaaccagttttccgttacttctgcatggaaaaattcttggatccagctatttggtttgagaagcgattggcttatacgcgcagtgtagctacttcttctattgttggttacatacttggacttggtgatagacatgtacagaatatcttgataaatgagcagtcagcagaacttgtacatatagatctaggtgttgcttttgaacagggcaaaatccttcctactcctgagacagttccttttagactcaccagagatattgtggatggcatgggcattacgggtgttgaaggtgtcttcagaagatgctgtgagaaaaccatggaagtgatgagaaactctcaggaaactctgttaaccattgtagaggtccttctatatgatccactctttgactggaccatgaatcctttgaaagctttgtatttacagcagaggccggaagatgaaactgagcttcaccctactctgaatgcagatgaccaagaatgcaaacgaaatctcagtgatattgaccagagtttcaacaaagtagctgaacgtgtcttaatgagactacaagagaaactgaaaggagtggaagaaggcactgtgctcagtgttggtggacaagtgaatttgctcatacagcaggccatagaccccaaaaatctcagccgacttttcccaggatggaaagcttgggtg

SEQ ID NO: 55 - DNA, 7932 bp, Homo sapiens
  misc_feature (1)..(7932): Homo sapiens ATR (NM_001184)
atgggggaacatggcctggagctggcttccatgatccccgccctgcgggagctgggcagtgccacaccagaggaatataatacagttgtacagaagccaagacaaattctgtgtcaattcattgaccggatacttacagatgtaaatgttgttgctgtagaacttgtaaagaaaactgactctcagccaacctccgtgatgttgcttgatttcatccagcatatcatgaaatcctccccacttatgtttgtaaatgtgagtggaagccatgaggccaaaggcagttgtattgaattcagtaattggatcataacgagacttctgcggattgcagcaactccctcctgtcatttgttacacaagaaaatctgtgaagtcatctgttcattattatttctttttaaaagcaagagtcctgctatttttggggtactcacaaaagaattattacaactttttgaagacttggtttacctccatagaagaaatgtgatgggtcatgctgtggaatggccagtggtcatgagccgatttttaagtcaattagatgaacacatgggatatttacaatcagctcctttgcagttgatgagtatgcaaaatttagaatttattgaagtcactttattaatggttcttactcgtattattgcaattgtgttttttagaaggcaagaactcttactttggcagataggttgtgttctgctagagtatggtagtccaaaaattaaatccctagcaattagctttttaacagaactttttcagcttggaggactaccagcacaaccagctagcacttttttcagctcatttttggaattattaaaacaccttgtagaaatggatactgaccaattgaaactctatgaagagccattatcaaagctgataaagacactatttccctttgaagcagaagcttatagaaatattgaacctgtctatttaaatatgctgctggaaaaactctgtgtcatgtttgaagacggtgtgctcatgcggcttaagtctgatttgctaaaagcagctttgtgccatttactgcagtatttccttaaatttgtgccagctgggtatgaatctgctttacaagtcaggaaggtctatgtgagaaatatttgtaaagctcttttggatgtgcttggaattgaggtagatgcagagtacttgttgggcccactttatgcagctttgaaaatggaaagtatggaaatcattgaggagattcaatgccaaactcaacaggaaaacctcagcagtaatagtgatggaatatcacccaaaaggcgtcgtctcagctcgtctctaaacccttctaaaagagcaccaaaacagactgaggaaattaaacatgtggacatgaaccaaaagagcatattatggagtgcactgaaacagaaagctgaatcccttcagatttcccttgaatacagtggcctaaagaatcctgttattgagatgttagaaggaattgctgttgtcttacaactgactgctctgtgtactgttcattgttctcatcaaaacatgaactgccgtactttcaaggactgtcaacataaatccaagaagaaaccttctgtagtgataacttggatgtcattggatttttacacaaaagtgcttaagagctgtagaagtttgttagaatctgttcagaaactggacctggaggcaaccattgataaggtggtgaaaatttatgatgctttgatttatatgcaagtaaacagttcatttgaagatcatatcctggaagatttatgtggtatgctctcacttccatggatttattcccattctgatgatggctgtttaaagttgaccacatttgccgctaatcttctaacattaagctgtaggatttcagatagctattcaccacaggcacaatcacgatgtgtgtttcttctgactctgtttccaagaagaatattccttgagtggagaacagcagtttacaactgggccctgcagagctcccatgaagtaatccgggctagttgtgttagtggattttttatcttattgcagcagcagaattcttgtaacagagttcccaagattcttatagataaagtcaaagatgattctgacattgtcaagaaagaatttgcttctatacttggtcaacttgtctgtactcttcacggcatgttttatctgacaagttctttaacagaacctttctctgaacacggacatgtggacctcttctgtaggaacttgaaagccacttctcaacatgaatgttcatcttctcaactaaaagcttctgtctgcaagccattccttttcctactgaaaaaaaaaatacctagtccagtaaaacttgctttcatagataatctacatcatctttgtaagcatcttgattttagagaagatgaaacagatgtaaaagcagttcttggaactttattaaatttaatggaagatccagacaaagatgttagagtggcttttagtggaaatatcaagcacatattggaatccttggactctgaagatggatttataaaggagctttttgtcttaagaatgaaggaagcatatacacatgcccaaatatcaagaaataatgagctgaaggataccttgattcttacaacaggggatattggaagggccgcaaaaggagatttggtaccatttgcactcttacacttattgcattgtttgttatccaagtcagcatctgtctctggagcagcatacacagaaattagagctctggttgcagctaaaagtgttaaactgcaaagttttttcagccagtataagaaacccatctgtcagtttttggtagaatcccttcactctagtcagatgacagcacttccgaatactccatgccagaatgctgacgtgcgaaaacaagatgtggctcaccagagagaaatggctttaaatacgttgtctgaaattgccaacgttttcgactttcctgatcttaatcgttttcttactaggacattacaagttctactacctgatcttgctgccaaagcaagccctgcagcttctgctctcattcgaactttaggaaaacaattaaatgtcaatcgtagagagattttaataaacaacttcaaatatattttttctcatttggtctgttcttgttccaaagatgaattagaacgtgcccttcattatctgaagaatgaaacagaaattgaactggggagcctgttgagacaagatttccaaggattgcataatgaattattgctgcgtattggagaacactatcaacaggtttttaatggtttgtcaatacttgcctcatttgcatccagtgatgatccatatcagggcccgagagatatcatatcacctgaactgatggctgattatttacaacccaaattgttgggcattttggctttttttaacatgcagttactgagctctagtgttggcattgaagataagaaaatggccttgaacagtttgatgtctttgatgaagttaatgggacccaaacatgtcagttctgtgagggtgaagatgatgaccacactgagaactggccttcgattcaaggatgattttcctgaattgtgttgcagagcttgggactgctttgttcgctgcctggatcatgcttgtctgggctcccttctcagtcatgtaatagtagctttgttacctcttatacacatccagcctaaagaaactgcagctatcttccactacctcataattgaaaacagggatgctgtgcaagattttcttcatgaaatatattttttacctgatcatccagaattaaaaaagataaaagccgttctccaggaatacagaaaggagacctctgagagcactgatcttcagacaactcttcagctctctatgaaggccattcaacatgaaaatgtcgatgttcgtattcatgctcttacaagcttgaaggaaaccttgtataaaaatcaggaaaaactgataaagtatgcaacagacagtgaaacagtagaacctattatctcacagttggtgacagtgcttttgaaaggttgccaagatgcaaactctcaagctcggttgctctgtggggaatgtttaggggaattgggggcgatagatccaggtcgattagatttctcaacaactgaaactcaaggaaaagattttacatttgtgactggagtagaagattcaagctttgcctatggattattgatggagctaacaagagcttaccttgcgtatgctgataatagccgagctcaagattcagctgcctatgccattcaggagttgctttctatttatgactgtagagagatggagaccaacggcccaggtcaccaattgtggaggagatttcctgagcatgttcgggaaatactagaacctcatctaaataccagatacaagagttctcagaagtcaaccgattggtctggagtaaagaagccaatttacttaagtaaattgggtagtaactttgcagaatggtcagcatcttgggcaggttatcttattacaaaggttcgacatgatcttgccagtaaaattttcacctgctgtagcattatgatgaagcatgatttcaaagtgaccatctatcttcttccacatattctggtgtatgtcttactgggttgtaatcaagaagatcagcaggaggtttatgcagaaattatggcagttctaaagcatgacgatcagcataccataaatacccaagacattgcatctgatctgtgtcaactcagtacacagactgtgttctccatgcttgaccatctcacacagtgggcaaggcacaaatttcaggcactgaaagctgagaaatgtccacacagcaaatcaaacagaaataaggtagactcaatggtatctactgtggattatgaagactatcagagtgtaacccgttttctagacctcataccccaggatactctggcagtagcttcctttcgctccaaagcatacacacgagctgtaatgcactttgaatcatttattacagaaaagaagcaaaatattcaggaacatcttggatttttacagaaattgtatgctgctatgcatgaacctgatggagtggccggagtcagtgcaattagaaaggcagaaccatctctaaaagaacagatccttgaacatgaaagccttggcttgctgagggatgccactgcttgttatgacagggctattcagctagaaccagaccagatcattcattatcatggtgtagtaaagtccatgttaggtcttggtcagctgtctactgttatcactcaggtgaatggagtgcatgctaacaggtccgagtggacagatgaattaaacacgtacagagtggaagcagcttggaaattgtcacagtgggatttggtggaaaactatttggcagcagatggaaaatctacaacatggagtgtcagactgggacagctattattatcagccaaaaaaagagatatcacagctttttatgactcactgaaactagtgagagcagaacaaattgtacctctttcagctgcaagctttgaaagaggctcctaccaacgaggatatgaatatattgtgagattgcacatgttatgtgagttggagcatagcatcaaaccacttttccagcattctccaggtgacagttctcaagaagattctctaaactgggtagctcgactagaaatgacccagaattcctacagagccaaggagcctatcctggctctccggagggctttactaagcctcaacaaaagaccagattacaatgaaatggttggagaatgctggctgcagagtgccagggtagctagaaaggctggtcaccaccagacagcctacaatgctctccttaatgcaggggaatcacgactcgctgaactgtacgtggaaagggcaaagtggctctggtccaagggtgatgttcaccaggcactaattgttcttcaaaaaggtgttgaattatgttttcctgaaaatgaaaccccacctgagggtaagaacatgttaatccatggtcgagctatgctactagtgggccgatttatggaagaaacagctaactttgaaagcaatgcaattatgaaaaaatataaggatgtgaccgcgtgcctgccagaatgggaggatgggcatttttaccttgccaagtactatgacaaattgatgcccatggtcacagacaacaaaatggaaaagcaaggtgatctcatccggtatatagttcttcattttggcagatctctacaatatggaaatcagttcatatatcagtcaatgccacgaatgttaactctatggcttgattatggtacaaaggcatatgaatgggaaaaagctggccgctccgatcgtgtacaaatgaggaatgatttgggtaaaataaacaaggttatcacagagcatacaaactatttagctccatatcaatttttgactgctttttcacaattgatctctcgaatttgtcattctcacgatgaagtttttgttgtcttgatggaaataatagccaaagtatttctagcctatcctcaacaagcaatgtggatgatgacagctgtgtcaaagtcatcttatcccatgcgtgtgaacagatgcaaggaaatcctcaataaagctattcatatgaaaaaatccttagagaagtttgttggagatgcaactcgcctaacagataagcttctagaattgtgcaataaaccggttgatggaagtagttccacattaagcatgagcactcattttaaaatgcttaaaaagctggtagaagaagcaacatttagtgaaatcctcattcctctacaatcagtcatgatacctacacttccatcaattctgggtacccatgctaaccatgctagccatgaaccatttcctggacattgggcctatattgcagggtttgatgatatggtggaaattcttgcttctcttcagaaaccaaagaagatttctttaaaaggctcagatggaaagttctacatcatgatgtgtaagccaaaagatgacctgagaaaggattgtagactaatggaattcaattccttgattaataagtgcttaagaaaagatgcagagtctcgtagaagagaacttcatattcgaacatatgcagttattccactaaatgatgaatgtgggattattgaatgggtgaacaacactgctggtttgagacctattctgaccaaactatataaagaaaagggagtgtatatgacaggaaaagaacttcgccagtgtatgctaccaaagtcagcagctttatctgaaaaactcaaagtattccgagaatttctcctgcccaggcatcctcctatttttcatgagtggtttctgagaacattccctgatcctacatcatggtacagtagtagatcagcttactgccgttccactgcagtaatgtcaatggttggttatattctggggcttggagaccgtcatggtgaaaatattctctttgattctttgactggtgaatgcgtacatgtagatttcaattgtcttttcaataagggagaaacctttgaagttccagaaattgtgccatttcgcctgactcataatatggttaatggaatgggtcctatgggaacagagggtctttttcgaagagcatgtgaagttacaatgaggctgatgcgtgatcagcgagagcctttaatgagtgtcttaaagacttttctacatgatcctcttgtggaatggagtaaaccagtgaaagggcattccaaagcgccactgaatgaaactggagaagttgtcaatgaaaaggccaagacccatgttcttgacattgagcagcgactacaaggtgtaatcaagactcgaaatagagtgacaggactgccgttatctattgaaggacatgtgcattaccttatacaggaagctactgatgaaaacttactatgccagatgtatcttggttggactccatatatg

SEQ ID NO: 56 - DNA, 12384 bp, Homo sapiens
  misc_feature (1)..(12384): Homo sapiens PRKDC (NM_006904)
atggcgggctccggagccggtgtgcgttgctccctgctgcggctgcaggagaccttgtccgctgcggaccgctgcggtgctgccctggccggtcatcaactgatccgcggcctggggcaggaatgcgtcctgagcagcagccccgcggtgctggcattacagacatctttagttttttccagagatttcggtttgcttgtatttgtccggaagtcactcaacagtattgaatttcgtgaatgtagagaagaaatcctaaagtttttatgtattttcttagaaaaaatgggccagaagatcgcaccttactctgttgaaattaagaacacttgtaccagtgtttatacaaaagatagagctgctaaatgtaaaattccagccctggaccttcttattaagttacttcagacttttagaagttctagactcatggatgaatttaaaattggagaattatttagtaaattctatggagaacttgcattgaaaaaaaaaataccagatacagttttagaaaaagtatatgagctcctaggattattgggtgaagttcatcctagtgagatgataaataatgcagaaaacctgttccgcgcttttctgggtgaacttaagacccagatgacatcagcagtaagagagcccaaactacctgttctggcaggatgtctgaaggggttgtcctcacttctgtgcaacttcactaagtccatggaagaagatccccagacttcaagggagatttttaattttgtactaaaggcaattcgtcctcagattgatctgaagagatatgctgtgccctcagctggcttgcgcctatttgccctgcatgcatctcagtttagcacctgccttctggacaactacgtgtctctatttgaagtcttgttaaagtggtgtgcccacacaaatgtagaattgaaaaaagctgcactttcagccctggaatcctttctgaaacaggtttctaatatggtggcgaaaaatgcagaaatgcataaaaataaactgcagtactttatggagcagttttatggaatcatcagaaatgtggattcgaacaacaaggagttatctattgctatccgtggatatggactttttgcaggaccgtgcaaggttataaacgcaaaagatgttgacttcatgtacgttgagctcattcagcgctgcaagcagatgttcctcacccagacagacactggtgacgaccgtgtttatcagatgccaagcttcctccagtctgttgcaagcgtcttgctgtaccttgacacagttcctgaggtgtatactccagttctggagcacctcgtggtgatgcagatagacagtttcccacagtacagtccaaaaatgcagctggtgtgttgcagagccatagtgaaggtgttcctagctttggcagcaaaagggccagttctcaggaattgcattagtactgtggtgcatcagggtttaatcagaatatgttctaaaccagtggtccttccaaagggccctgagtctgaatctgaagaccaccgtgcttcaggggaagtcagaactggcaaatggaaggtgcccacatacaaagactacgtggatctcttcagacatctcctgagctctgaccagatgatggattctattttagcagatgaagcatttttctctgtgaattcctccagtgaaagtctgaatcatttactttatgatgaatttgtaaaatccgttttgaagattgttgagaaattggatcttacacttgaaatacagactgttggggaacaagagaatggagatgaggcgcctggtgtttggatgatcccaacttcagatccagcggctaacttgcatccagctaaacctaaagatttttcggctttcattaacctggtggaattttgcagagagattctccctgagaaacaagcagaattttttgaaccatgggtgtactcattttcatatgaattaattttgcaatctacaaggttgcccctcatcagtggtttctacaaattgctttctattacagtaagaaatgccaagaaaataaaatatttcgagggagttagtccaaagagtctgaaacactctcctgaagacccagaaaagtattcttgctttgctttatttgtgaaatttggcaaagaggtggcagttaaaatgaagcagtacaaagatgaacttttggcctcttgtttgacctttcttctgtccttgccacacaacatcattgaactcgatgttagagcctacgttcctgcactgcagatggctttcaaactgggcctgagctatacccccttggcagaagtaggcctgaatgctctagaagaatggtcaatttatattgacagacatgtaatgcagccttattacaaagacattctcccctgcctggatggatacctgaagacttcagccttgtcagatgagaccaagaataactgggaagtgtcagctctttctcgggctgcccagaaaggatttaataaagtggtgttaaagcatctgaagaagacaaagaacctttcatcaaacgaagcaatatccttagaagaaataagaattagagtagtacaaatgcttggatctctaggaggacaaataaacaaaaatcttctgacagtcacgtcctcagatgagatgatgaagagctatgtggcctgggacagagagaagcggctgagctttgcagtgccctttagagagatgaaacctgtcattttcctggatgtgttcctgcctcgagtcacagaattagcgctcacagccagtgacagacaaactaaagttgcagcctgtgaacttttacatagcatggttatgtttatgttgggcaaagccacgcagatgccagaagggggacagggagccccacccatgtaccagctctataagcggacgtttcctgtgctgcttcgacttgcgtgtgatgttgatcaggtgacaaggcaactgtatgagccactagttatgcagctgattcactggttcactaacaacaagaaatttgaaagtcaggatactgttgccttactagaagctatattggatggaattgtggaccctgttgacagtactttaagagatttttgtggtcggtgtattcgagaattccttaaatggtccattaagcaaataacaccacagcagcaggagaagagtccagtaaacaccaaatcgcttttcaagcgactttatagccttgcgcttcaccccaatgctttcaagaggctgggagcatcacttgcctttaataatatctacagggaattcagggaagaagagtctctggtggaacagtttgtgtttgaagccttggtgatatacatggagagtctggccttagcacatgcagatgagaagtccttaggtacaattcaacagtgttgtgatgccattgatcacctatgccgcatcattgaaaagaagcatgtttctttaaataaagcaaagaaacgacgtttgccgcgaggatttccaccttccgcatcattgtgtttattggatctggtcaagtggcttttagctcattgtgggaggccccagacagaatgtcgacacaaatccattgaactcttttataaattcgttcctttattgccaggcaacagatcccctaatttgtggctgaaagatgttctcaaggaagaaggtgtctcttttctcatcaacacctttgaggggggtggctgtggccagccctcgggcatcctggcccagcccaccctcttgtaccttcgggggccattcagcctgcaggccacgctatgctggctggacctgctcctggccgcgttggagtgctacaacacgttcattggcgagagaactgtaggagcgctccaggtcctaggtactgaagcccagtcttcacttttgaaagcagtggctttcttcttagaaagcattgccatgcatgacattatagcagcagaaaagtgctttggcactggggcagcaggtaacagaacaagcccacaagagggagaaaggtacaactacagcaaatgcaccgttgtggtccggattatggagtttaccacgactctgctaaacacctccccggaaggatggaagctcctgaagaaggacttgtgtaatacacacctgatgagagtcctggtgcagacgctgtgtgagcccgcaagcataggtttcaacatcggagacgtccaggttatggctcatcttcctgatgtttgtgtgaatctgatgaaagctctaaagatgtccccatacaaagatatcctagagacccatctgagagagaaaataacagcacagagcattgaggagctttgtgccgtcaacttgtatggccctgacgcgcaagtggacaggagcaggctggctgctgttgtgtctgcctgtaaacagcttcacagagctgggcttctgcataatatattaccgtctcagtccacagatttgcatcattctgttggcacagaacttctttccctggtttataaaggcattgcccctggagatgagagacagtgtctgccttctctagacctcagttgtaagcagctggccagcggacttctggagttagcctttgcttttggaggactgtgtgagcgccttgtgagtcttctcctgaacccagcggtgctgtccacggcgtccttgggcagctcacagggcagcgtcatccacttctcccatggggagtatttctatagcttgttctcagaaacgatcaacacggaattattgaaaaatctggatcttgctgtattggagctcatgcagtcttcagtggataataccaaaatggtgagtgccgttttgaacggcatgttagaccagagcttcagggagcgagcaaaccagaaacaccaaggactgaaacttgcgactacaattctgcaacactggaagaagtgtgattcatggtgggccaaagattcccctctcgaaactaaaatggcagtgctggccttactggcaaaaattttacagattgattcatctgtatcttttaatacaagtcatggttcattccctgaagtctttacaacatatattagtctacttgctgacacaaagctggatctacatttaaagggccaagctgtcactcttcttccattcttcaccagcctcactggaggcagtctggaggaacttagacgtgttctggagcagctcatcgttgctcacttccccatgcagtccagggaatttcctccaggaactccgcggttcaataattatgtggactgcatgaaaaagtttctagatgcattggaattatctcaaagccctatgttgttggaattgatgacagaagttctttgtcgggaacagcagcatgtcatggaagaattatttcaatccagtttcaggaggattgccagaaggggttcatgtgtcacacaagtaggccttctggaaagcgtgtatgaaatgttcaggaaggatgacccccgcctaagtttcacacgccagtcctttgtggaccgctccctcctcactctgctgtggcactgtagcctggatgctttgagagaattcttcagcacaattgtggtggatgccattgatgtgttgaagtccaggtttacaaagctaaatgaatctacctttgatactcaaatcaccaagaagatgggctactataagattctagacgtgatgtattctcgccttcccaaagatgatgttcatgctaaggaatcaaaaattaatcaagttttccatggctcgtgtattacagaaggaaatgaacttacaaagacattgattaaattgtgctacgatgcatttacagagaacatggcaggagagaatcagctgctggagaggagaagactttaccattgtgcagcatacaactgcgccatatctgtcatctgctgtgtcttcaatgagttaaaattttaccaaggttttctgtttagtgaaaaaccagaaaagaacttgcttatttttgaaaatctgatcgacctgaagcgccgctataattttcctgtagaagttgaggttcctatggaaagaaagaaaaagtacattgaaattaggaaagaagccagagaagcagcaaatggggattcagatggtccttcctatatgtcttccctgtcatatttggcagacagtaccctgagtgaggaaatgagtcaatttgatttctcaaccggagttcagagctattcatacagctcccaagaccctagacctgccactggtcgttttcggagacgggagcagcgggaccccacggtgcatgatgatgtgctggagctggagatggacgagctcaatcggcatgagtgcatggcgcccctgacggccctggtcaagcacatgcacagaagcctgggcccgcctcaaggagaagaggattcagtgccaagagatcttccttcttggatgaaattcctccatggcaaactgggaaatccaatagtaccattaaatatccgtctcttcttagccaagcttgttattaatacagaagaggtctttcgcccttacgcgaagcactggcttagccccttgctgcagctggctgcttctgaaaacaatggaggagaaggaattcactacatggtggttgagatagtggccactattctttcatggacaggcttggccactccaacaggggtccctaaagatgaagtgttagcaaatcgattgcttaatttcctaatgaaacatgtctttcatccaaaaagagctgtgtttagacacaaccttgaaattataaagacccttgtcgagtgctggaaggattgtttatccatcccttataggttaatatttgaaaagttttccggtaaagatcctaattctaaagacaactcagtagggattcaattgctaggcatcgtgatggccaatgacctgcctccctatgacccacagtgtggcatccagagtagcgaatacttccaggctttggtgaataatatgtcctttgtaagatataaagaagtgtatgccgctgcagcagaagttctaggacttatacttcgatatgttatggagagaaaaaacatactggaggagtctctgtgtgaactggttgcgaaacaattgaagcaacatcagaatactatggaggacaagtttattgtgtgcttgaacaaagtgaccaagagcttccctcctcttgcagacaggttcatgaatgctgtgttctttctgctgccaaaatttcatggagtgttgaaaacactctgtctggaggtggtactttgtcgtgtggagggaatgacagagctgtacttccagttaaagagcaaggacttcgttcaagtcatgagacatagagatgatgaaagacaaaaagtatgtttggacataatttataagatgatgccaaagttaaaaccagtagaactccgagaacttctgaaccccgttgtggaattcgtttcccatccttctacaacatgtagggaacaaatgtataatattctcatgtggattcatgataattacagagatccagaaagtgagacagataatgactcccaggaaatatttaagttggcaaaagatgtgctgattcaaggattgatcgatgagaaccctggacttcaattaattattcgaaatttctggagccatgaaactaggttaccttcaaataccttggaccggttgctggcactaaattccttatattctcctaagatagaagtgcactttttaagtttagcaacaaattttctgctcgaaatgaccagcatgagcccagattatccaaaccccatgttcgagcatcctctgtcagaatgcgaatttcaggaatataccattgattctgattggcgtttccgaagtactgttctcactccgatgtttgtggagacccaggcctcccagggcactctccagacccgtacccaggaagggtccctctcagctcgctggccagtggcagggcagataagggccacccagcagcagcatgacttcacactgacacagactgcagatggaagaagctcatttgattggctgaccgggagcagcactgacccgctggtcgaccacaccagtccctcatctgactccttgctgtttgcccacaagaggagtgaaaggttacagagagcacccttgaagtcagtggggcctgattttgggaaaaaaaggctgggccttccaggggacgaggtggataacaaagtgaaaggtgcggccggccggacggacctactacgactgcgcagacggtttatgagggaccaggagaagctcagtttgatgtatgccagaaaaggcgttgctgagcaaaaacgagagaaggaaatcaagagtgagttaaaaatgaagcaggatgcccaggtcgttctgtacagaagctaccggcacggagaccttcctgacattcagatcaagcacagcagcctcatcaccccgttacaggccgtggcccagagggacccaataattgcaaaacagctctttagcagcttgttttctggaattttgaaagagatggataaatttaagacactgtctgaaaaaaacaacatcactcaaaagttgcttcaagacttcaatcgttttcttaataccaccttctctttctttccaccctttgtctcttgtattcaggacattagctgtcagcacgcagccctgctgagcctcgacccagcggctgttagcgctggttgcctggccagcctacagcagcccgtgggcatccgcctgctagaggaggctctgctccgcctgctgcctgctgagctgcctgccaagcgagtccgtgggaaggcccgcctccctcctgatgtcctcagatgggtggagcttgctaagctgtatagatcaattggagaatacgacgtcctccgtgggatttttaccagtgagataggaacaaagcaaatcactcagagtgcattattagcagaagccagaagtgattattctgaagctgctaagcagtatgatgaggctctcaataaacaagactgggtagatggtgagcccacagaagccgagaaggatttttgggaacttgcatcccttgactgttacaaccaccttgctgagtggaaatcacttgaatactgttctacagccagtatagacagtgagaaccccccagacctaaataaaatctggagtgaaccattttatcaggaaacatatctaccttacatgatccgcagcaagctgaagctgctgctccagggagaggctgaccagtccctgctgacatttattgacaaagctatgcacggggagctccagaaggcgattctagagcttcattacagtcaagagctgagtctgctttacctcctgcaagatgatgttgacagagccaaatattacattcaaaatggcattcagagttttatgcagaattattctagtattgatgtcctcttacaccaaagtagactcaccaaattgcagtctgtacaggctttaacagaaattcaggagttcatcagctttataagcaaacaaggcaatttatcatctcaagttccccttaagagacttctgaacacctggacaaacagatatccagatgctaaaatggacccaatgaacatctgggatgacatcatcacaaatcgatgtttctttctcagcaaaatagaggagaagcttacccctcttccagaagataatagtatgaatgtggatcaagatggagaccccagtgacaggatggaagtgcaagagcaggaagaagatatcagctccctgatcaggagttgcaagttttccatgaaaatgaagatgatagacagtgcccggaagcagaacaatttctcacttgctatgaaactactgaaggagctgcataaagagtcaaaaaccagagacgattggctggtgagctgggtgcagagctactgccgcctgagccactgccggagccggtcccagggctgctctgagcaggtgctcactgtgctgaaaacagtctctttgttggatgagaacaacgtgtcaagctacttaagcaaaaatattctggctttccgtgaccagaacattctcttgggtacaacttacaggatcatagcgaatgctctcagcagtgagccagcctgccttgctgaaatcgaggaggacaaggctagaagaatcttagagctttctggatccagttcagaggattcagagaaggtgatcgcgggtctgtaccagagagcattccagcacctctctgaggctgtgcaggcggctgaggaggaggcccagcctccctcctggagctgtgggcctgcagctggggtgattgatgcttacatgacgctggcagatttctgtgaccaacagctgcgcaaggaggaagagaatgcatcagttattgattctgcagaactgcaggcgtatccagcacttgtggtggagaaaatgttgaaagctttaaaattaaattccaatgaagccagattgaagtttcctagattacttcagattatagaacggtatccagaggagactttgagcctcatgacaaaagagatctcttccgttccctgctggcagttcatcagctggatcagccacatggtggccttactggacaaagaccaagccgttgctgttcagcactctgtggaagaaatcactgataactacccgcaggctattgtttatcccttcatcataagcagcgaaagctattccttcaaggatacttctactggtcataagaataaggagtttgtggcaaggattaaaagtaagttggatcaaggaggagtgattcaagattttattaatgccttagatcagctctctaatcctgaactgctctttaaggattggagcaatgatgtaagagctgaactagcaaaaacccctgtaaataaaaaaaacattgaaaaaatgtatgaaagaatgtatgcagccttgggtgacccaaaggctccaggcctgggggcctttagaaggaagtttattcagacttttggaaaagaatttgataaacattttgggaaaggaggttctaaactactgagaatgaagctcagtgacttcaacgacattaccaacatgctacttttaaaaatgaacaaagactcaaagccccctgggaatctgaaagaatgttcaccctggatgagcgacttcaaagtggagttcctgagaaatgagctggagattcccggtcagtatgacggtaggggaaagccattgccagagtaccacgtgcgaatcgccgggtttgatgagcgggtgacagtcatggcgtctctgcgaaggcccaagcgcatcatcatccgtggccatgacgagagggaacaccctttcctggtgaagggtggcgaggacctgcggcaggaccagcgcgtggagcagctcttccaggtcatgaatgggatcctggcccaagactccgcctgcagccagagggccctgcagctgaggacctatagcgttgtgcccatgacctccaggttaggattaattgagtggcttgaaaatactgttaccttgaaggaccttcttttgaacaccatgtcccaagaggagaaggcggcttacctgagtgatcccagggcaccgccgtgtgaatataaagattggctgacaaaaatgtcaggaaaacatgatgttggagcttacatgctaatgtataagggcgctaatcgtactgaaacagtcacgtcttttagaaaacgagaaagtaaagtgcctgctgatctcttaaagcgggccttcgtgaggatgagtacaagccctgaggctttcctggcgctccgctcccacttcgccagctctcacgctctgatatgcatcagccactggatcctcgggattggagacagacatctgaacaactttatggtggccatggagactggcggcgtgatcgggatcgactttgggcatgcgtttggatccgctacacagtttctgccagtccctgagttgatgccttttcggctaactcgccagtttatcaatctgatgttaccaatgaaagaaacgggccttatgtacagcatcatggtacacgcactccgggccttccgctcagaccctggcctgctcaccaacaccatggatgtgtttgtcaaggagccctcctttgattggaaaaattttgaacagaaaatgctgaaaaaaggagggtcatggattcaagaaataaatgttgctgaaaaaaattggtacccccgacagaaaatatgttacgctaagagaaagttagcaggtgccaatccagcagtcattacttgtgatgagctactcctgggtcatgagaaggcccctgccttcagagactatgtggctgtggcacgaggaagcaaagatcacaacattcgtgcccaagaaccagagagtgggctttcagaagagactcaagtgaagtgcctgatggaccaggcaacagaccccaacatccttggcagaacctgggaaggatgggagccctggatg

SEQ ID NO: 57 - DNA, 1428 bp, Homo sapiens
  misc_feature (1)..(1428): Homo sapiens CHEK1 (NM_001114122)
atggcagtgccctttgtggaagactgggacttggtgcaaaccctgggagaaggtgcctatggagaagttcaacttgctgtgaatagagtaactgaagaagcagtcgcagtgaagattgtagatatgaagcgtgccgtagactgtccagaaaatattaagaaagagatctgtatcaataaaatgctaaatcatgaaaatgtagtaaaattctatggtcacaggagagaaggcaatatccaatatttatttctggagtactgtagtggaggagagctttttgacagaatagagccagacataggcatgcctgaaccagatgctcagagattcttccatcaactcatggcaggggtggtttatctgcatggtattggaataactcacagggatattaaaccagaaaatcttctgttggatgaaagggataacctcaaaatctcagactttggcttggcaacagtatttcggtataataatcgtgagcgtttgttgaacaagatgtgtggtactttaccatatgttgctccagaacttctgaagagaagagaatttcatgcagaaccagttgatgtttggtcctgtggaatagtacttactgcaatgctcgctggagaattgccatgggaccaacccagtgacagctgtcaggagtattctgactggaaagaaaaaaaaacatacctcaacccttggaaaaaaatcgattctgctcctctagctctgctgcataaaatcttagttgagaatccatcagcaagaattaccattccagacatcaaaaaagatagatggtacaacaaacccctcaagaaaggggcaaaaaggccccgagtcacttcaggtggtgtgtcagagtctcccagtggattttctaagcacattcaatccaatttggacttctctccagtaaacagtgcttctagtgaagaaaatgtgaagtactccagttctcagccagaaccccgcacaggtctttccttatgggataccagcccctcatacattgataaattggtacaagggatcagcttttcccagcccacatgtcctgatcatatgcttttgaatagtcagttacttggcaccccaggatcctcacagaacccctggcagcggttggtcaaaagaatgacacgattctttaccaaattggatgcagacaaatcttatcaatgcctgaaagagacttgtgagaagttgggctatcaatggaagaaaagttgtatgaatcaggttactatatcaacaactgataggagaaacaataaactcattttcaaagtgaatttgttagaaatggatgataaaatattggttgacttccggctttctaagggtgatggattggagttcaagagacacttcctgaagattaaagggaagctgattgatattgtgagcagccagaagatttggcttcctgccaca

SEQ ID NO: 58 - DNA, 1629 bp, Homo sapiens
  misc_feature (1)..(1629): Homo sapiens CHEK2 (NM_007194)
atgtctcgggagtcggatgttgaggctcagcagtctcatggcagcagtgcctgttcacagccccatggcagcgttacccagtcccaaggctcctcctcacagtcccagggcatatccagctcctctaccagcacgatgccaaactccagccagtcctctcactccagctctgggacactgagctccttagagacagtgtccactcaggaactctattctattcctgaggaccaagaacctgaggaccaagaacctgaggagcctacccctgccccctgggctcgattatgggcccttcaggatggatttgccaatcttgaatgtgtgaatgacaactactggtttgggagggacaaaagctgtgaatattgctttgatgaaccactgctgaaaagaacagataaataccgaacatacagcaagaaacactttcggattttcagggaagtgggtcctaaaaactcttacattgcatacatagaagatcacagtggcaatggaacctttgtaaatacagagcttgtagggaaaggaaaacgccgtcctttgaataacaattctgaaattgcactgtcactaagcagaaataaagtttttgtcttttttgatctgactgtagatgatcagtcagtttatcctaaggcattaagagatgaatacatcatgtcaaaaactcttggaagtggtgcctgtggagaggtaaagctggctttcgagaggaaaacatgtaagaaagtagccataaagatcatcagcaaaaggaagtttgctattggttcagcaagagaggcagacccagctctcaatgttgaaacagaaatagaaattttgaaaaagctaaatcatccttgcatcatcaagattaaaaacttttttgatgcagaagattattatattgttttggaattgatggaagggggagagctgtttgacaaagtggtggggaataaacgcctgaaagaagctacctgcaagctctatttttaccagatgctcttggctgtgcagtaccttcatgaaaacggtattatacaccgtgacttaaagccagagaatgttttactgtcatctcaagaagaggactgtcttataaagattactgattttgggcactccaagattttgggagagacctctctcatgagaaccttatgtggaacccccacctacttggcgcctgaagttcttgtttctgttgggactgctgggtataaccgtgctgtggactgctggagtttaggagttattctttttatctgccttagtgggtatccacctttctctgagcataggactcaagtgtcactgaaggatcagatcaccagtggaaaatacaacttcattcctgaagtctgggcagaagtctcagagaaagctctggaccttgtcaagaagttgttggtagtggatccaaaggcacgttttacgacagaagaagccttaagacacccgtggcttcaggatgaagacatgaagagaaagtttcaagatcttctgtctgaggaaaatgaatccacagctctaccccaggttctagcccagccttctactagtcgaaagcggccccgtgaaggggaagccgagggtgccgagaccacaaagcgcccagctgtgtgtgctgctgtgttg

SEQ ID NO: 59 - DNA, 2538 bp, Homo sapiens
  misc_feature (1)..(2538): Homo sapiens EXO1 (NM_006027)
atggggatacagggattgctacaatttatcaaagaagcttcagaacccatccatgtgaggaagtataaagggcaggtagtagctgtggatacatattgctggcttcacaaaggagctattgcttgtgctgaaaaactagccaaaggtgaacctactgataggtatgtaggattttgtatgaaatttgtaaatatgttactatctcatgggatcaagcctattctcgtatttgatggatgtactttaccttctaaaaaggaagtagagagatctagaagagaaagacgacaagccaatcttcttaagggaaagcaacttcttcgtgaggggaaagtctcggaagctcgagagtgtttcacccggtctatcaatatcacacatgccatggcccacaaagtaattaaagctgcccggtctcagggggtagattgcctcgtggctccctatgaagctgatgcgcagttggcctatcttaacaaagcgggaattgtgcaagccataattacagaggactcggatctcctagcttttggctgtaaaaaggtaattttaaagatggaccagtttggaaatggacttgaaattgatcaagctcggctaggaatgtgcagacagcttggggatgtattcacggaagagaagtttcgttacatgtgtattctttcaggttgtgactacctgtcatcactgcgtgggattggattagcaaaggcatgcaaagtcctaagactagccaataatccagatatagtaaaggttatcaagaaaattggacattatctcaagatgaatatcacggtaccagaggattacatcaacgggtttattcgggccaacaataccttcctctatcagctagtttttgatcccatcaaaaggaaacttattcctctgaacgcctatgaagatgatgttgatcctgaaacactaagctacgctgggcaatatgttgatgattccatagctcttcaaatagcacttggaaataaagatataaatacttttgaacagatcgatgactacaatccagacactgctatgcctgcccattcaagaagtcatagttgggatgacaaaacatgtcaaaagtcagctaatgttagcagcatttggcataggaattactctcccagaccagagtcgggtactgtttcagatgccccacaattgaaggaaaatccaagtactgtgggagtggaacgagtgattagtactaaagggttaaatctcccaaggaaatcatccattgtgaaaagaccaagaagtgcagagctgtcagaagatgacctgttgagtcagtattctctttcatttacgaagaagaccaagaaaaatagctctgaaggcaataaatcattgagcttttctgaagtgtttgtgcctgacctggtaaatggacctactaacaaaaagagtgtaagcactccacctaggacgagaaataaatttgcaacatttttacaaaggaaaaatgaagaaagtggtgcagttgtggttccagggaccagaagcaggtttttttgcagttcagattctactgactgtgtatcaaacaaagtgagcatccagcctctggatgaaactgctgtcacagataaagagaacaatctgcatgaatcagagtatggagaccaagaaggcaagagactggttgacacagatgtagcacgtaattcaagtgatgacattccgaataatcatattccaggtgatcatattccagacaaggcaacagtgtttacagatgaagagtcctactcttttgagagcagcaaatttacaaggaccatttcaccacccactttgggaacactaagaagttgttttagttggtctggaggtcttggagatttttcaagaacgccgagcccctctccaagcacagcattgcagcagttccgaagaaagagcgattcccccacctctttgcctgagaataatatgtctgatgtgtcgcagttaaagagcgaggagtccagtgacgatgagtctcatcccttacgagaagaggcatgttcttcacagtcccaggaaagtggagaattctcactgcagagttcaaatgcatcaaagctttctcagtgctctagtaaggactctgattcagaggaatctgattgcaatattaagttacttgacagtcaaagtgaccagacctccaagctacgtttatctcatttctcaaaaaaagacacacctctaaggaacaaggttcctgggctatataagtccagttctgcagactctctttctacaaccaagatcaaacctctaggacctgccagagccagtgggctgagcaagaagccggcaagcatccagaagagaaagcatcataatgccgagaacaagccggggttacagatcaaactcaatgagctctggaaaaactttggatttaaaaaagattctgaaaagcttcctccttgtaagaaacccctgtccccagtcagagataacatccaactaactccagaagcggaagaggatatatttaacaaacctgaatgtggccgtgttcaaagagcaatattccag

SEQ ID NO: 60 - DNA, 18 bp, Cricetulus griseus
  misc_feature (1)..(18): Telomeric repeats (CHO chromosome 9 and 15)
ttagggttagggttaggg

(19) United States
(12) Patent Application Publication
(10) Pub. No.: US 2023/0046668 A1
(43) Pub. Date: Feb. 16, 2023

(54) TARGETED INTEGRATION IN MAMMALIAN SEQUENCES ENHANCING GENE EXPRESSION

(71) Applicant: Selexis S.A., Plan-Les-Ouates (CH)

(72) Inventors: Pierre-Olivier Duroy, Plan-Les-Ouates (CH); Alexandre Regamey, Plan-les-Ouates (CH); Ghislaine Arib, Plan-les-Ouates (CH); Valerie Le Fourn, Plan-Les-Ouates (CH); David Calabrese, Plan-les-Ouates (CH); Pierre-Alain Girod, Plan-les-Ouates (CH)

(73) Assignee: Selexis S.A., Plan-les-Ouates (CH)

(21) Appl. No.: 17/788,548

(22) PCT Filed: Dec. 24, 2020
(86) PCT No.: PCT/IB2020/062436

§ 371 Date: Jun. 23, 2022

Related U.S. Application Data
(60) Provisional application No. 62/960,367, filed on Jan. 13, 2020.
(60) Provisional application No. 62/953,405, filed on Dec. 24, 2019.

(51) Int. Cl. C12N 15/90; C12N 15/10; C12N 9/22; C12N 5/071

(52) U.S. Cl. CPC C12N 15/907; C12N 15/102; C12N 9/22; C12N 5/0682; C12N 2310/20; C12N 2740/10022

(57) ABSTRACT

Disclosed are cells that have stably integrated into their genomes exogenous nucleic acid sequences, such as transgenes, within or proximal to the integration site of a sequence comprising at least part of an endogenous retrovirus (ERV) or a LTR-retrotransposon (LTR-RT), or instead of a sequence encompassing an ERV or a LTR-RT that is part or was part of the genome of the cell, as well as method of producing and using such cells. Advantageously, a high level and/or stable production of the transgene expression product(s) can be achieved. Transgene integration and expression may be furthered by modulating the DNA repair pathways of the cell, e.g., by transiently expressing a gene encoding a protein that forms part of a DNA repair pathway during transgene integration.

### BACKGROUND OF THE INVENTION

Expression of recombinant proteins in mammalian cells is of great importance for biotechnological production of recombinant proteins and/or for therapeutic uses such as gene and cell therapies. The generation of respective cell lines requires the successful integration of the transgene into the host genome and then its expression in said cells. Currently, mainstream strategies for cell line development depend on i) random integration of the transgene into the chromosomes of the cells, ii) the selection of cells having the transgene integrated and iii) the selection of specific cells presenting optimal productivity characteristics. However, this approach is limited by the number of transgene copies integrated and by epigenetic effects of the genomic environment of the transgene often causing low, unstable transcription and/or high clonal variability.

To overcome these issues commonly associated with cell line development, epigenetic regulators can be used to protect transgenes from negative position effects (Bell and Felsenfeld, 1999). These epigenetic regulators include boundary or insulator elements, locus control regions (LCRs), stabilizing and antirepressor (STAR) elements, ubiquitously acting chromatin opening elements (UCOE) and matrix attachment regions (MARs). All of these epigenetic regulators have been used for recombinant protein production in mammalian cell lines (Zahn-Zabal et al., 2001; Kim et al., 2004) and for gene therapies (Agarwal et al., 1998; Castilla et al., 1998).

The publications and other materials, including patents and patent applications, used herein to illustrate the invention and, in particular, to provide additional details respecting the practice of the invention are incorporated herein by reference in their entirety. For convenience, the publications are referenced in the following text either by a number for reference to the appended bibliography, by the name of the authors and year published or by the patent/patent publication number.

There is a need for site-specific targeted integration of transgenes that are suitable for, among others, increasing and stabilizing transgene expression in mammalian cells. Site-specific targeted integration of transgenes is also needed since it advantageously also leads to cells having identical genomic set ups, eliminating the need for the screening of many cell clones in order to identify and select those having a high level of transgene expression. There is also a need for suitable integration site(s) for specific subclasses of transgenes called “landing pad(s).” These integration site(s) advantageously ensure the stability of the integrated cell line and a long-term high expression rate, from a single transgene or from low copy numbers. Thus, there is in particular a need in the art to identify and validate suitable insertion sites for transgenes in mammalian cells, for efficient and reliable transgene expression. There is also a need for cell clones used for therapeutic protein production that lack expressed endogenous retroviral sequences (ERV) and/or do not or to a lesser extent release viral particles into the cell supernatant, together with the produced therapeutic protein. One or more of the above mentioned needs as well as other needs are addressed herein.

### SUMMARY OF THE INVENTION

Disclosed is, among others, the stable integration of exogenous nucleic acid sequences, such as transgenes, within or proximal to the insertion sequence of at least part of an endogenous retroviral sequences (ERV) or a LTR-retrotransposon (LTR-RT) of mammalian genome. In certain embodiments this results in high level and/or stable production of transgene expression product(s). This is in certain embodiments accomplished and/or furthered by modulating the DNA repair pathways of the host cell.

Disclosed is an engineered cell, preferably of a mammalian cell line such as an engineered CHO cell, including an engineered CHO-K1 cell, an engineered porcine cell or an engineered human cell comprising:
a genome of the cell comprising:
at least one locus comprising an insertion site of an ERV sequence or LTR-RT sequence, and
a transgene integrated into the at least one locus.

The at least one locus and optionally the corresponding allelic locus into which the transgene is integrated may be an ERV sequence or a LTR-RT sequence insertion locus.

The at least one locus into which the transgene is integrated may be an allelic wild-type (e.g. ERV-devoid or LTR-RT-devoid) counterpart locus of an ERV sequence insertion locus or a LTR-RT sequence insertion locus (e.g. the ERV-integrated or LTR-RT-integrated genomic sequences). The transgene may also be integrated adjacent to or replace the corresponding ERV sequence or the corresponding LTR-RT sequence at the insertion locus. The transgene may be integrated into either one or both loci, preferably in more than 20%, 30% or even more than 40% of the transgene-containing cells within a cell population. The locus may be homozygous and comprise at least two copies of, e.g, SEQ ID NO: 1 or SEQ ID NO: 2 or parts thereof. The locus may be heterozygous and comprise, e.g., both SEQ ID NO: 1 and SEQ ID NO: 2 or parts thereof.

Certain embodiments are directed to an engineered cell, preferably of a mammalian cell line such as an engineered CHO cell, including an engineered CHO-K1 cell, an engineered porcine cell or an engineered human cell comprising:

within the genome of the cell:

at least one locus comprising an insertion site of an endogenous retrovirus (ERV) sequence or a LTR-retrotransposon (LTR-RT) sequence, wherein said at least one locus comprise(s):
(i) a) the ERV sequence or the LTR-RT sequence, or b) the insertion site and, optionally, parts of the sequence of a), and/or
(ii) an allelic wild type counterpart sequence of (i), and

at least one transgene encoding at least one transgene expression product integrated into the at least one locus. The cell may comprise (i) and (ii) on different chromosomes, such as chromosome 15 and 9 of a CHO cell. The cell may comprise (i) and (ii), and the at least one transgene may be integrated into (ii), but not (i) or also into (i).

Certain embodiments are directed to a cell population comprising engineered cells described herein. The at least one transgene may be integrated into more than 20%, 30% or even more than 40% of (i) and/or (ii) of cells within said cell population. The engineered cells of the cell population may comprise (i) and (ii), above. (1) may comprise: at least nucleotides 29021 to 40247 (or 29521 to 39747) of SEQ ID NO: 1 or a sequence having 95%, 98% or 99% sequence identity with nucleotides 29021 to 40247 (or 29521 to 39747) of SEQ ID NO: 1 and (ii) may comprise at least nucleotides 29020 to 31020 (or 29520 to 30520) of SEQ ID NO: 2 or a sequence having 95% 98%, or 99% sequence identity with nucleotides 29020 to 31020 (or 29520 to 30520) of SEQ ID NO: 2. The engineered cell/cell population in certain preferred embodiments lacks expressed endogenous retroviral sequences (ERV). In certain preferred embodiments there are no detectable viral particles comprised in the cell population culture supernatant.

The at least one transgene expression product may be a product of interest such as a protein of interest. The cell/cell population may optionally express the product/protein of interest per unit of time, in an amount (such as picograms per cell and per day, μg/l or mg/l) that exceeds the amount of a product/protein of interest when the at least one transgene is integrated into the genome outside the at least one locus, by at least 1.5 fold, 2 fold, 2.5 fold, 3 fold or more. The ERV or LTR-RT may be selected from the group consisting of a type C endoretroviral element (ERV C), MLV (murine leukemia virus), XMRV (xenotropic murine leukemia virus-related virus), MMTV (mouse mammary tumor virus), MERV-L (mouse ERV with L-tRNA PBS), VL30 (virus like 30), IAP (intracisternal A-type particle), MusD (Mus type-D related retrovirus), PERVs (porcine endogenous retroviruses), KoRV (koala retrovirus), enJSRV (Jaagsiekte sheep retrovirus), MaLR (mammalian apparent LTR retrotransposons), HERV (Human endogenous retroviruses) such as HERV-E (human ERV with E-tRNA PBS), HERV-H (human ERV with H-tRNA PBS), HERV-K (human ERV with K-tRNA PBS), HERV-L (human ERV with L-tRNA PBS), HERV-W (human ERV with W-tRNA PBS) and combinations thereof.

The ERV or LTR-RT sequence may comprise at least one ERV subsequence selected from the group consisting of a gag (group-specific antigen) gene, a pol (polymerase) gene, a env (envelope) gene, a sequence encoding a MA (matrix), a CA (capsid), a NC (nucleocapsid), a sequence encoding a SP1 (Spacer peptide 1), a sequence encoding a SP2 (Spacer peptide 2) or a further domain encoding proteins such as pp12 or p6, are long terminal repeats (LTRs) of a ERV and combinations thereof and wherein the transgene is optionally integrated into one of the subsequences.

The cell may be transfected with one or more vectors comprising one or more genes of Table 2 or SEQ ID Nos: 25-28, 38-58 and/or 59 or sequences having at least 80%, 85%, 90%, 95%, 98% or 99% sequence identity with SEQ ID Nos: 25-28, 38-58 and/or 59, wherein the cytoplasm of the cell(s) may optionally further comprise(s) exogenous chemical inhibitors and/or stimulators of one or more DNA Repair Pathways (DRPs), such as NHEJ inhibitors selected from the group consisting of NU7441, Olaparib, DNA Ligase IV inhibitor, Scr7 KU-0060648 anti-EGFR-antibody C225 (Cetuximab), Compound 401 (2-(4-Morpholinyl)-4H-pyrimido[2,I-a]isoquinolin-4-one), Vanillin, Wortmannin, DMNB, IC87361, LY294002, OK-1035, CO 15, NK314, PI 103 hydrochloride and combinations thereof, MMEJ inhibitors selected from the group of Mirin, derivatives of Mirin, inhibitors of PoIQ, inhibitors of CtIP and combinations thereof, HR inhibitors such as RI-1 and B02, HR stimulators such as RS-1, NHEJ stimulators, such as IP6 and combinations of any one of the above inhibitors and/or stimulators. In any of the engineered cells the locus may have at least 80%, 90%, 95%, 96%, 97%, 98% or 99% sequence identity with a sequence selected from SEQ ID Nos. 1 and/or SEQ ID No. 2. The transgene may be a landing pad. The engineered cell is in certain preferred embodiments a Chinese Hamster Ovary (CHO) cell, or a human cell or a porcine cell.

One embodiment comprises a method for transgene integration into a genome of a cell, preferably of a mammalian cell line comprising:

(a) providing at least one transgene as part of a vector, such as a plasmid or viral vector, comprising the at least one transgene, wherein the vector integrates the transgene into a least one locus of the cell comprising an insertion site of an endogenous retrovirus (ERV) sequence or a LTR-retrotransposon (LTR-RT) sequence, or

(b) providing at least one transgene, optionally as part of a vector, and at least one nuclease and/or nickase, wherein the nuclease and/or nickase is preferably encoded by at least one vector, wherein the nuclease and/or nickase introduces, for integration of said transgene therein, double and/or single strand breaks into a least one locus of the cell comprising an insertion site of an endogenous retrovirus (ERV) sequence or a LTR-retrotransposon (LTR-RT) sequence and optionally, providing at least one vector encoding at least one targeting element guiding said least one nuclease and/or nickase,

optionally, upmodulating, in particular stimulating at least one first DNA Repair Pathway (DRP) of the cell and optionally downmodulating, in particular stimulating at least one second DRP of the cell, or vice versa,

transfecting the cell with the at least one transgene; and

optionally isolating an engineered cell that comprises the transgene integrated into the locus. The cell/cell line may also be transfected with, preferably as part of one or more further vector(s), one or more genes of Table 2 or SEQ ID Nos: 25-28, 38-58 and/or 59 or sequences having at least 80%, 85%, 90%, 95%, 98% or 99% sequence identity with SEQ ID Nos: 25-28, 38-58 and/or 59 and/or brought into contact with a chemical affecting the DNA Repair Pathway (DRP) of the cell. The cell maybe also be transfected with one or more further vector(s) comprising and expressing SEQ ID Nos: 25-28, 38-58 and/or 59, preferably SEQ ID Nos: 25-28.

The at least one nuclease and/or nickase may be a transposase, an integrase, a recombinase such as a site-specific recombinase, a nickase, or a nuclease such as a site-specific nuclease, a fusion protein comprising a programmable DNA-binding domain and a nuclease domain or any combinations thereof, or

a homing endonuclease, a restriction enzyme, a zinc-finger nuclease or a zinc-finger nickase, a meganuclease or a meganickase, a transcription activator-like effector nuclease or a transcription activator-like effector nickase, an RNA-guided nuclease or an RNA-guided nickase, a DNA-guided nuclease or a DNA-guided nickase, a megaTAL nuclease, a BurrH-nuclease, an ARCUS nuclease, a modified or chimeric version or variant thereof, and combinations thereof, in particular a zinc-finger nuclease or a zinc-finger nickase, a transcription activator-like effector nuclease or a transcription activator-like effector nickase, a RNA-guided nuclease or an RNA-guided nickase, wherein the RNA-guided nuclease or an RNA-guided nickase is optionally part of a CRISPR-based system, restriction enzyme and combinations thereof. The recombinase may be a Cre recombinase, FLP recombinase, lambda integrase, PhiC31 integrase, Dre recombinase, xb1 integrase, gamma delta resolvase, R4 integrase, Tn3 resolvase, or TP901-1 recombinase. In certain embodiments, the nuclease is a transcription activator-like effector nuclease or a RNA-guided nuclease.

The vectors used herein may be plasmids or viral vectors such as an AAV vector.

The first and/or second DRP may be selected from the group consisting of resection, canonical homology directed repair (canonical HDR), homologous recombination (HR), alternative homology directed repair (Alt-HDR), double-strand break repair (DSBR), single-strand annealing (SSA), synthesis-dependent strand annealing (SDSA), break-induced replication (BIR), alternative end-joining (Alt-EJ), microhomology mediated end-joining (MMEJ), DNA synthesis-dependent microhomology-mediated end-joining (SD-MMEJ), canonical non-homologous end-joining repair (C-NHEJ), alternative non-homologous end joining (A-NHEJ), translesion DNA synthesis repair (TLS), base excision repair (BER), nucleotide excision repair (NER), mismatch repair (MMR), DNA damage responsive (DDR), blunt end joining, single strand break repair (SSBR), interstrand crosslink repair (ICL), Fanconi Anemia (FA) Pathway and combinations thereof.

In certain embodiments, the at least one first DRP is homologous recombination (HR) and the at least one second DRP is one or more non-homologous end joining (NHEJ) DNA Repair pathway; the at least one first DRP may be an Alt-EJ pathway such as MMEJ, and the at least one second DRP may be one or more non-homologous end joining (NHEJ) DNA Repair pathway; the at least one first DRP may be an Alt-EJ pathway such as MMEJ, and the at least one second DRP may be a homologous recombination (HR) DNA Repair pathway; or the at least one first DRP may be an Alt-EJ pathway such as MMEJ, and the at least one second DRP may be one or more alternative DNA repair pathway.

An interference with/alternation of the DRP may be an upmodulation of the same and may take the form of a) expressing, including causing overexpression of at least one component of the DRP in said cell, b) introducing into said cell, at least one component of the said DRP, and/or c) contacting said cell, with at least one stimulator such as a chemical stimulator of a component of the DRP, such as HR stimulator(s) such as RS-1 and/or NHEJ stimulator(s), such as IP6.

An interference with/alternation of may be a downmodulation and may take the form of a) contacting said cell, with at least one inhibitor such as a chemical inhibitor, such as NHEJ inhibitor selected from the group of NU7441, Olaparib, DNA Ligase IV inhibitor, Scr7 KU-0060648 anti-EGFR-antibody C225 (Cetuximab), Compound 401 (2-(4-Morpholinyl)-4H-pyrimido[2,I-a]isoquinolin-4-one), Vanillin, Wortmannin, DMNB, IC87361, LY294002, OK-1035, CO 15, NK314, PI 103 hydrochloride and combinations thereof, MMEJ inhibitors selected from the group of Mirin, derivatives of Mirin, inhibitors of PoIQ, inhibitors of CtIP and combinations thereof, HR inhibitors such as RI-1 and/or BO2, of a component of the DRP,

b) inactivating or downregulating at least one component of the DRP, by contacting or expressing in said cell, at least one inhibitory nucleic acid such as a miRNA, a siRNA, a shRNA, and/or

c) expressing in said cell a protein that inhibits the said DRP, or any combination thereof.

One embodiment comprises an engineered cell produced by the one of the methods disclosed herein.

Another embodiment comprises a kit for introducing at least one transgene into a cell comprising:

in one container a vector encoding a nuclease and/or nickase targeting at least one locus comprising an insertion site of an endogenous retrovirus (ERV) sequence or a LTR-retrotransposon (LTR-RT) sequence, such as SEQ ID NOs: 1 and 2, preferably a locus comprising (i) nucleotides 29021 to 40247 of SEQ ID NO: 1 or a sequence having 95%, 98% or 99% sequence identity with nucleotides 29021 to 40247 of SEQ ID NO: 1 and (ii) at least nucleotides 29020 to 31020 of SEQ ID NO: 2 or a sequence having 95% 98%, or 99% sequence identity with nucleotides 29020 to 31020 of SEQ ID NO: 2, or a locus comprising nucleotides 29521 to 39747 of SEQ ID NO: 1 or a sequence having 95%, 98% or 99% sequence identity with nucleotides 29521 to 4247 of SEQ ID NO: 1 and (ii) comprising nucleotides 29520 to 30520 of SEQ ID NO: 2 or a sequence having 95% 98%, or 99% sequence identity with nucleotides 29520 to 30520 of SEQ ID NO: 2, including an ERV sequence or a LTR-RT sequence integrated into the insertion site, such as SEQ ID NO: 3 and, optionally at least one vector encoding at least one targeting element guiding said least one nuclease and/or nickase,

optionally, in a separate container at least one vector encoding at least one targeting element guiding said least one nuclease and/or nickase,

in a separate container: at least one stimulator and/or inhibitor a DNA Repair Pathway (DRP), and/or

one or more vectors comprising one or more genes encoding one or more of the DRP proteins of Table 2 or SEQ ID Nos: 25-28, 38-58 and/or 59 or sequences having at least 80%, 85%, 90%, 95%, 98% or 99% sequence identity with SEQ ID Nos: 25-28, 38-58 and/or 59;

and instructions how to transfect the cell with the transgene using the at least one nuclease and/or nickase and the at least one stimulator and/or inhibitor.

### BRIEF DESCRIPTION OF THE DRAWINGS

FIG. 1 is a schematic representation of a locus with integration site, showing one integration site comprising an ERV sequence, here the ERV C 109F (SEQ ID NO: 3) containing allele, and one wild-type counterpart allele thereof.

FIGS. 2A and 2B show a double CRISPR-based approach for targeting transgene integration in the ERV C 109F locus, in the wild type allele (FIG. 2A) and in the ERV C 109F allele (FIG. 2B).

FIGS. 3A and 3B show vectors used for the targeted integration into the ERV C 109F allele (FIG. 3A) or into the wild type allele (FIG. 3B), using a vector without homology sequences, with or without stimulation of the Alt-EJ repair pathway.

FIGS. 4A and 4B show vectors used for the targeted integration into the ERV C 109F allele (FIG. 4A) or into the wild type allele (FIG. 4B), using a vector with homology sequences, with or without stimulation of the HR repair pathway.

FIGS. 5A and 5B show a TaqMan qPCR assay development for the wild type allele (FIG. 5A) and the ERV C 109F allele (FIG. 5B). The boxed thick horizontal lines indicate the positions of the TaqMan qPCR assay amplicons.

FIG. 6 shows a percentage of clones comprising a transgene integration in both alleles, in the WT allele, in the ERV C 109F allele or transgene integration at a random locus, either with or without DNA homology sequences, and either with or without DPR stimulation.

FIGS. 7A, 7B and 7C show a fluorescent in-situ hybridization using a transgene probe for the detection of integration events at the ERV locus. Notably due to chromosomal rearrangement one “ERV locus” can be found in two different chromosomes of the CHO cell (chromosome 15 and 9).

FIGS. 8A, 8B, 8C and 8D show GFP Fluorescence measurements for each type of cell clones: No DNA homology/Alt-EJ stimulation (FIG. 8A), No DNA homology/No Alt-EJ stimulation (FIG. 8B), DNA Homology/HR stimulation (FIG. 8C) and DNA Homology/No HR stimulation (FIG. 8D) (compare to FIG. 6).

FIG. 9 is a comparison of GFP fluorescence results obtained for the clones using targeting integration with DNA sequence homologies on the transgene vector, comparing the HR mechanism modulation to the absence of HR mechanism modulation.

FIGS. 10A, 10B and 10C are a representation of the integration site of the Type-C ERV 109F sequence on Chromosome 15 (FIG. 10A) and its allelic wild-type counterpart locus on chromosome 9 (FIG. 10B). CRISPR cleavage sites are indicated by triangles under the DNA sequences the top panels or by boxes in FIG. 10C at the bottom.

FIGS. 11A and 11B show vectors used for the transfection to generate Trastuzumab-producing CHO cell clones. In FIG. 11A on the left, the co-transfected vectors for transient expression are shown. In FIG. 11B the immunoglobulin (Ig) expression vectors are shown, namely the vectors carrying Tras_Hc (heavy chain) and Tras_Lc (light chain) sequences and their integration sites at chromosome 15 and 9, respectively.

FIGS. 12A, 12B, 12C and 12D are a schematic representation of the 4 types of clones characterized after targeted integration of the transgene in CHO-M cells at the ERV109F genomic locus. FIG. 12A: Transgene is integrated at both alleles of the locus (in the Figures referred to as “integrated at both loci” or just “both”) (ERV109F allele of chromosome 15 and wild type allele at chromosome 9); FIG. 12B: Transgene is integrated at the ERV109F allele of chromosome 15, but not at the wild type allele of chromosome 9 (in the Figures referred to as “integrated at ERV locus” or just “ERV”); FIG. 12C: The transgene is integrated at wild type allele of chromosome 9, but not at the ERV109F allele of chromosome 15 (in the Figures referred to as “integrated at the locus without ERV” or just “ERV devoid”); and FIG. 12D: the transgene did not integrate at either allele of the locus, but integrated randomly into the host chromosome. Grey arrows represent the PCR primers used for characterization of the clone transgene genomic integration sites.

FIGS. 13A, 13B and 13C show the fold decrease of ERV C 109F expression following transgene integration versus the parental CHO-M cells. Total RNA was extracted from the indicated cell clones, and the viral RNA levels were determined by RT-qPCR and processed using the Delta Delta Ct (cycle threshold) calculation method. The hatching corresponds to the hatching shown in FIGS. 12A-12C. FIG. 13A depict low titers cultures, FIG. 13B medium titers cultures, and FIG. 13C high titers cultures.

FIGS. 14A, 14B and 14C show the determination of trastuzumab production levels in the supernatant of various CHO cells clone types. ELISA assays were performed on the cell-free supernatant of 3-day cultures in 96-well plates, for clone types having incorporated the Tras expression construct at the indicated genomic “loci” (alleles). The panels indicate the titers of Trastuzumab antibody obtained from cultures displaying low (FIG. 14A), medium (FIG. 14B) and high (FIG. 14C) levels of production for each clone type.

FIGS. 15A-F provide measures of the clone's productivity of Trastuzumab during a fed-batch culture of 10 days in 24 deep-wells plate (FIGS. 15A (low titers), 15B (medium titers), and 15C (high titers) and with 3 ml of medium culture with a starting material of 300 000 cells/ml, or during cultures performed with 96-well plates FIGS. 15D (low titers), 15E (medium titers), and 15F (high titers). Tras protein titer measurements were performed using a LabChip LCGXII System® (PERKIN ELMER, Inc.). The hatching corresponds in designation to the hatching shown in FIGS. 13A-13C and 14A-14C: In each graph from left to right: Transgene integrated at both alleles of the locus “(both”) (ERV109F allele of chromosome 15 and wild type allele at chromosome 9); Transgene is integrated at the ERV109F allele of chromosome 15, but not at the wild type allele of chromosome 9 (“ERV”); Transgene did not integrate at either allele of the locus, but integrated randomly into the host chromosome, Transgene is integrated at wild type allele of chromosome 9, but not at the ERV109F allele of chromosome 15 (“ERV devoid”).

FIGS. 16A and 16B depict the testing of the highly producing 4 clones from FIG. 15C (FIG. 16A) and FIG. 15F (FIG. 16B) in an Ambr®15 automated microscale bioreactor system (SARTORIUS Stedim, Germany) for 14 days to assess their productivity at a larger scale.

### DESCRIPTION OF VARIOUS AND PREFERRED EMBODIMENTS OF THE INVENTION

Disclosed herein are transgene producing cells and cell lines and methods of making and using them. For the production of a transgene of interest, one or more ERV or LTR-RT locus/loci in the genome of the cell are targeted for transgene integration and expression. ERV sequences that are capable to form viral particles may in certain embodiments be eliminated or at least are made or have been made non-functional in terms of viral particle production. An ERV or LTR-RT locus targeted may comprise one allele that in fact comprises the ERV or LTR-RT sequence (or contained the ERV or LTR-RT sequence prior to removal), while the other allele does not and is a so-called wild type allele and never contained a ERV or LTR-RT sequence. The transgene(s) may be introduced into the cell to preferably integrate at an ERV or LTR-RT locus into the allele that comprises the ERV or LTR-RT sequence, into the allele that does not comprises the ERV or LTR-RT sequence or in both.

In one example the transgenes encode an antibiotic selection gene and a gene encoding a protein of interest such as the heavy and light chains of an immunoglobulin or human erythropoietin. The transgenes are inserted into a vector comprising promoter upstream of the transgenes and an SGE (Selexis Genetic Element) downstream of the transgenes. Transcription activator-like effector (TALE) nickases are engineered to recognize and cut specific sequences of DNA at the ERV locus 5 bps upstreams of the ERV integration site and 5 bps downstream of the ERV integration site. The selected ERV is only integrated at the locus in one of the two alleles, the other allele is a so-called wild type allele that never contained the ERV. A CHO-K1 cell is transfected with vectors carrying the gene encoding the TALE nickases, the vectors carrying the transgene as well as a vector designed for the transient expression of MRE11. Cells that show transgene integration into the allelic wild-type counterpart locus/allele of an ERV sequence, but not into the allele containing the ERV are selected for production of the protein of interest.

In another example a kit is used to create CHO cells producing a transgene of interest. The CHO cells of the kit has been engineered to remove any integrated ERV sequences that produce viral particles or virus like particles. The cell has also been engineered to insert a landing pad in the allelic wild-type counterpart locus/allele of an ERV sequence. The landing pad encodes Green Fluorescent Protein (GFP). The kit also includes a vector encoding a nickase for a sequence in the landing pad as well as at least one vector encoding at least one targeting element guiding said least one nickase to the landing pad. The kit also includes a vector designed for the transient expression of CIRBP as well as a vector into which the transgene of interest can be integrated. After integrating the transgene of interest, which is a single domain antibody, all vectors are co-transfected into the engineered CHO cell. CHO cells that do not express GFP are selected. The expression vector for RS-1, which is a RAD51 stimulator, is also part of the kit and is added during co-transfection to stimulate homologous recombination (HR).

A cell/cell population (the latter is often also referred to as cells of a cell line indicating the homogenous nature of the cells in a cell population) according to the present invention is an eukaryotic, preferably mammalian cell/cell population, a such as a human or non-human mammalian cell, capable of being maintained under cell culture conditions. A non-limiting example of this type of cell are human cells such as HEK cells (Human embryonic kidney), Chinese hamster ovary (CHOs) cells, mouse myeloma cells, including NS0 and Sp2/0 cells, porcine cells such as LLCPK (porcine kidney epithelial) cell. Modified versions of CHO cell include CHO DG44, CHO-K1 and CHO pro-3. In one preferred embodiment a SURE CHO-M Cell™ line (SELEXIS SA, Switzerland) is used.

An insertion site of an endogenous retrovirus (ERV) sequence or a LTR-retrotransposon (LTR-RT) sequence of a genome of a cell is a nucleic acid sequence having a length of not more than 100 nucleotides, preferably not more than 90, 80, 70, 60, 50, 40, 30, 20, 10, 5, 4, 3, 2 nucleotides: (i) a) comprising an ERV or a LTR-RT sequence, i.e. a ERV or a LTR-RT sequence integrated into the genome of the cell, which is also referred to herein as an integrated ERV/LTR-RT sequence, (i) b) having comprised an ERV or a LTR-RT sequence prior to complete or partial removal of the ERV a LTR-RT sequence or (ii) is the allelic wild type, sometimes referred to herein as ERV-devoid, counterpart locus of (i). (i) a) and b) are referred to herein as “ERV sequence or LTR-RT sequence insertion locus/allele” or “ERV or LTR-RT insertion locus/allele”, (ii) is referred to herein as “allelic wild-type counterpart locus/allele of an ERV sequence insertion locus/allele” or the “allelic wild-type counterpart locus/allele of an LTR-RT sequence insertion locus/allele” or just “allelic wild-type (wt) counterpart sequence” of the above. As a person skilled in the art will readily understand, there will be loci in which:
one allele
(i) a) comprises an ERV or a LTR-RT sequence, or
(i) b) has comprised an ERV or a LTR-RT sequence prior to complete or partial removal of the ERV or LTR-RT sequence, and/or
(ii) is the allelic wild type counterpart of (i).

A cell that combines at least two distinct alleles of a locus, e.g., (i) a) and (ii), or (i) b) and (ii), is sometimes referred to herein as heterozygous for that locus.

A cell that combines (i) a) and (ii) is said to be hemizygous for the single copy ERV sequence. A cell having two identical alleles at a locus, e.g. (i) a) and (i) a), is referred to as homozygous for that locus.

Also within the scope of the invention are cells:
in which one allele and the corresponding allelic locus, ergo both alleles (i) a) comprise an ERV or a LTR-RT sequence or (i) b) have comprised an ERV or a LTR-RT sequence prior to complete or partial removal of the ERV a LTR-RT sequence; and
in which one allele and the corresponding allelic locus, ergo both alleles (ii) are allelic wild type counterpart loci an ERV sequence or a LTR-RT sequence insertion locus.

One non-limiting example of such an insertion site of an ERV sequence is contained in SEQ ID NO: 1, 2, and at the 3′ end of SEQ ID NO: 4 and the 5′ end of SEQ ID NO: 5. The ERV sequence is shown in SEQ ID NO: 3.

An allelic wild type counterpart locus of (i) that comprises the corresponding 100, 90, 80, 70, 60, 50, 40, 30, 20, 10, 5, 4, 3, 2 nucleotides of the integration site, but no evidence of a current or past ERV or LTR-RT sequence integration. A non-limiting example of such an allelic counterpart to SEQ ID NO: 1 is SEQ ID NO: 2 and the insertion site are the nucleotides around nucleotide 30020. SEQ ID NO; 1 would be “ERV sequence or LTR-RT sequence insertion locus”, while SEQ ID NO: 2 would be the corresponding “allelic wild-type counterpart locus of an ERV sequence or a LTR-RT sequence insertion locus.”

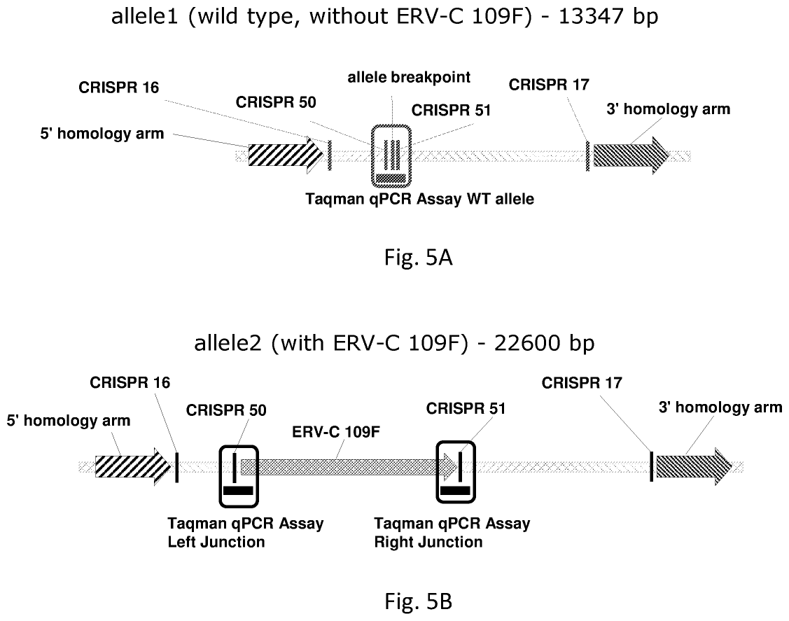

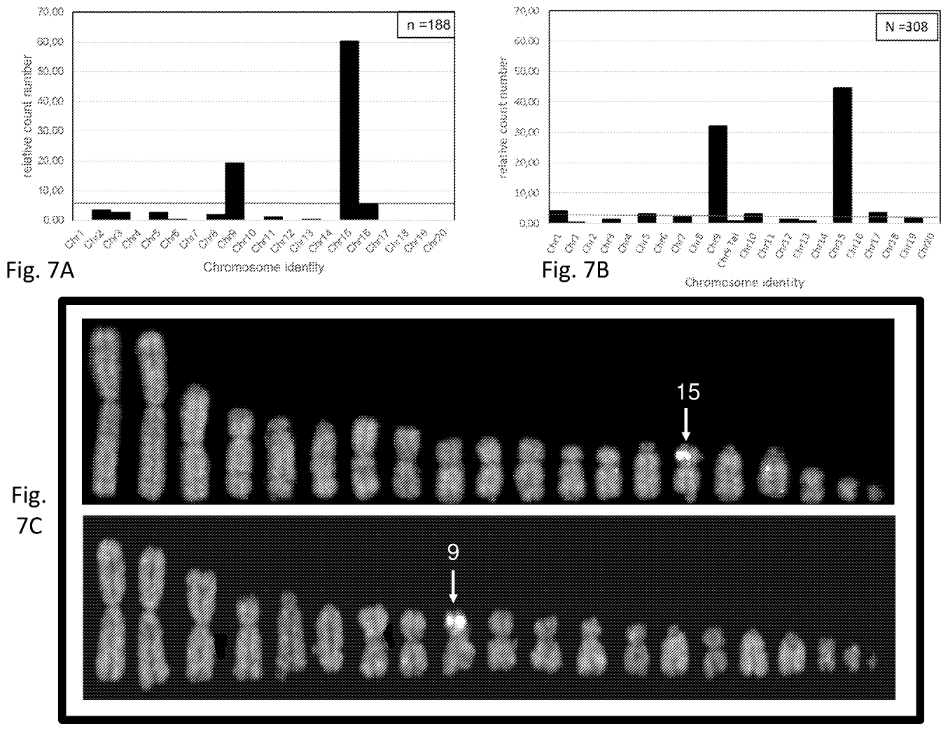

A locus is a position on a chromosome of an eukaryotic cell where a specific genomic sequence having in the present context up generally to 60000 nucleotides (see FIGS. 5 A and B) but not less than 1000, 900, 800, 700 or 600 nucleotides of the genome is located. However, as the person skilled in the art knows, in, e.g., the human genome loci having a length between 612 and 4 767 747 base pairs could be identified (Taher and Ovcharenko, 2009). An allele is a particular form of a genetic locus, distinguished from other forms by its particular nucleotide sequence. In cells that are subject to intense genomic rearrangement, which are many cells capable of being maintained under cell culture conditions and used in transgene product production, such a locus can also be found on different chromosomes due to this genomic rearrangement. To capture these configurations that share genomic sequences of generally 1000-60000 nucleotides that define a locus, the different alleles of such a locus may be referred to as, e.g., “allelic wild-type counterpart locus of an ERV sequence or a LTR-RT sequence insertion locus” and “ERV sequence or LTR-RT sequence insertion locus” as discussed elsewhere herein. An example for such a locus is the locus with the insertion site for ERV-C 109F. The locus exists, due to the rearrangement, on chromosomes 15 and 9 (see also FIGS. 7A-7C). On chromosome 15, the ERV-C 109F sequences is integrated into the genome, while on chromosome 7, the “allelic wild-type counterpart locus” is situated. The fact that this is in effect one locus that exists on 2 chromosomes can be deduced from the corresponding surrounding sequences 5′ and/or 3′ of the insertion site, e.g., for ERV-C 109F, e.g. SEQ ID NO: 1 and SEQ ID NO: 2, e.g., nucleotides 1-30020 of SEQ ID NO: 1 and nucleotides 1-30020 of SEQ ID NO: 2. Thus a multi-chromosome locus has high sequence identities, often 100% such as for the upstream sequences of the ERV-C 109F insertion site (nucleotides 1-30020 of SEQ ID NO: 1 and nucleotides 1-30020 of SEQ ID NO: 2). However, slight mismatches are possible lowering the sequence identity to e.g., 99%, 98%, 97%, 96% or 95%. Also, there might be deletions around the ERV sequence or LTR-RT sequence insertion site.

A locus comprising an insertion site of an endogenous retrovirus (ERV) sequence or a LTR-retrotransposon (LTR-RT) sequence is generally identifiable by or is a sequence up to 60000, 50000, 40000, 30000, however generally less than 20000 or 10000 nucleotides, in certain instances less than 9000, 8000, 7000, 6000, 5000, 4000, 3000, 2000 or is identifiable by or is a sequence of 1000 to 600 nucleotides, including about 900, 800, 700 nucleotides. As noted above, one such locus is shown in SEQ ID NO: 1 and its corresponding allelic wild type counterpart locus is shown in SEQ ID NO: 2. As the person skilled in the art will understand, it is within the scope of the present invention that an integration of, e.g., a transgene can occur within any part of the sequence of an endogenous retrovirus (ERV) sequence (see, e.g., SEQ ID NO: 3) or a LTR-retrotransposon (LTR-RT), or within the chromosomal sequences of the locus flanking the ERV or LTR-RT sequences (ERV or LTR-RT flanking sequences), or replace a fully or partially deleted ERV or LTR-RT sequence or ERV or LTR-RT flanking sequences of the locus (see, e.g., SEQ ID NOs: 4 and/or 5). A preferred locus comprising the insertion site of an ERV sequence or a LTR-RT sequence is a locus in which an ERV sequence comprising at least part of but preferably a complete gag gene, a pol gene, an env gene, and/or at least one, preferably two LTR(s), most preferably all of these subsequences of an ERV or the respective subsequences of a LTR-RT. In certain embodiments, an even more preferred locus comprising the insertion site of an endogenous retrovirus (ERV) sequence or a LTR-retrotransposon (LTR-RT) sequence is the corresponding allelic wild-type counterpart locus, that is devoid of any ERV sequence or LTR-RT sequence.

As the person skilled in the art will appreciate, ERV sequences other than those shown in SEQ ID Nos: 1-5 as well as LTR-RT sequences and their respective loci are also within the scope of the present invention. Some non-limiting examples such ERV sequences are listed in Table 1 as SEQ ID Nos: 12-24.

TABLE 1
Alternative ERVs commonly found in in CHO cells.

| SEQ ID NO: | Examples of alternative ERVs in CHO cells |
|---|---|
| 12 | 000036F-021-01:1-25000_Type_C:ERV C 36F |
| 13 | 000066F-017-01:1-39146_Type_C:ERV C 66F |
| 14 | 000132F-023-01:1-15000_Type_C:ERV C 132F |
| 15 | 000161F-025-01:1-17947_Type_C:ERV C 161F |
| 16 | 000308F-002-01:1-31438_Type_C:ERV C 308F |
| 17 | 000312F-023-01:15000-35000_Type_C:ERV C 312F |
| 18 | 000322F-036-01:15000-35000_Type_C:ERV C 322F |
| 19 | 000351F-017-01:1-31837_Type_C:ERV C 351F |
| 20 | 000519F-009-01:1-25000_Type_C:ERV C 519F |
| 21 | 000562F-005-01:25000-45000_Type_C:ERV C 62F |
| 22 | 001329F-001-01:1-4677_Type_C:ERV C1329F |
| 23 | 000386F:294053-310687_Type_C:expressed ERV |
| | type C 368F |
| 24 | 000506F:340000-360000_Type_C:expressed ERV |
| | type C 506F |

As noted, a locus and its insertion site comprising an ERV sequence or a LTR-RT sequence has an allelic counterpart locus that also comprises the insertion site but may or may not comprise an ERV sequence or a LTR-RT sequence. As also noted above, in certain embodiments none of the loci may have an ERV sequence or a LTR-RT sequence: The cell may have been or may be engineered to remove the ERV sequence or the LTR-RT sequence or parts thereof and in certain embodiments also parts of the locus, i.e. sequences flanking the ERV or LTR-RT insertion site/the ERV or LTR-RT sequences inserted therein. The cell might, at the time of transgene integration be engineered accordingly, or, alternatively, the transgene might be integrated into a cell that has already been engineered to remove or alter parts of ERV sequence or a LTR-RT sequences. Respective alterations and the resulting cells are disclosed, e.g., in U.S. Patent application 62/784,566 and international patent application publication WO2020/136149, designating the US, which are incorporated herein by reference in their entireties.

Hemizygous with respect to the ERV sequence/LTR-RT sequence refers to the fact that there is only one copy of a given ERV sequence/LTR-RT sequence in a specific locus of a diploid cell. That means that there is a “ERV sequence or LTR-RT sequence insertion locus” and an “allelic wild-type counterpart locus of an ERV sequence or a LTR-RT sequence insertion locus.” Homozygous with respect to the ERV sequence/LTR-RT sequence means that the alleles of the locus correspond and either both carry the “ERV sequence or LTR-RT sequence insertion” or are both “allelic wild-type counterparts” in a specific locus of a diploid cell.

A transgene can be integrated into a locus that is hemi- or homozygous with respect to ERV sequence/LTR-RT sequence.

An LTR-retrotransposon (LTR-RT) sequence also referred to as Mammalian LTR-retrotransposon sequence or MaLR sequence comprises at least two LTR sequences that flank a region encoding two enzymes: at least a gag gene and a pol gene that can be translated for at least two enzymes, integrase and reverse transcriptase (RT). In contrast to ERVs, LTR-RT sequences never contain an env gene that encodes an envelope protein (ENV) (Havecker et al., 2004).

An endogenous retrovirus (ERV) sequence constitutes a left-over of retroviral integration into the genome of a cell and comprise at least parts of a gag gene, a pol gene, and an env gene, and/or at least one, preferably two LTR(s). Functional units or parts thereof that make up a ERV sequences are also referred to as ERV subsequences. Thus, a gag gene, a pol gene, an env gene, an LTR are considered an ERV subsequence. In a preferred embodiment, at least one, preferably two of the gag gene, the pol gene or the env gene expresses the respective protein. In an even more preferred embodiment of ERV selection, the ERV sequence releases VPs (viral particles) or a VLPs (viral like particles). The size of a complete endogenous retrovirus is between 6-12 kb on average and it contains gag, pol and env genes that always occur in the same order. Coding sequences are flanked by two LTRs (Long Terminal Repeat sequences). Most ERVs are defective, as they are carrying a multitude of inactivating mutations. In addition, they can be inactivated (i.e. not transcribed) by epigenetic silencing effects. However, some ERVs still have open reading frames in their genome and/or they may be transcriptionally active. The ERVs of mammals bear strong similarities and may originate from the genus of gammaretroviruses and betaretroviruses, including Intracisternal type-A particle (IAP or IAPS), Feline leukemia virus (FeLV), Mouse Leukemia Virus (MLV), Koala epidemic virus (KoRV), Mouse Mammary Tumor Virus (MMTV). ERVs are maintained in the genomes and may have certain advantages for the cells into whose genome they are integrated, including providing a source of genetic diversity and protection against other viral pathogens. However, they can become infectious and carry risks in in the context of transgene, i.e. protein, expression described elsewhere herein, in particular, as a result of ERV awakening due to cancer, cellular stress and/or epigenetic modifications.

The three major proteins encoded within the retroviral genome are Gag, Pol, and Env. Gag (Group Antigens) encoded by the gag gene is a polyprotein, which is processed to matrix and other core proteins, including the nucleoprotein core particle, that determines the retroviral core. Pol is the reverse transcriptase, encoded by the pol gene and has RNase H and integrase function. Its activity results in the double-stranded DNA pre-integrated form of the virus and, via the integrase function, for the integration into the host genome, and also via the RNase function, the reverse transcription after integration into the genome of the host. Env is the envelope protein, encoded by the env gene, and resides in the lipid layer of the virus determining the viral tropism.

The gag gene gives rise to a Gag precursor protein, which is expressed from the unspliced viral mRNA. The Gag precursor protein is cleaved by the virally encoded protease (a product of the pol gene) during the process of viral maturation into generally four smaller proteins designated MA (matrix), CA (capsid), NC (nucleocapsid), and a further protein domain (e.g. pp12 in murine leukemia virus (MLV) or p6 in HIV).

The viral protease (Pro), integrase (IN), RNase H, and reverse transcriptase (RT) are expressed within the context of a Gag-Pol fusion protein. The Gag-Pol precursor is generally generated by a ribosomal frame shifting event, which is triggered by a specific cis-acting RNA motif (a heptanucleotide sequence followed by a short stem loop in the distal region of the Gag RNA). When ribosomes encounter this motif, they shift approximately 5% of the time to the pol reading frame without interrupting translation. The frequency of ribosomal frameshifting explains why the Gag and the Gag-Pol precursor are produced at a ratio of approximately 20:1.

During viral maturation, the virally encoded protease cleaves the Pol polypeptide away from Gag and further digests it to separate the protease, RT, RNase H, and integrase activities. These cleavages do not all occur efficiently, for example, roughly 50% of the RT protein remains linked to RNase H as a single polypeptide (p65) (Hope & Trono, 2000).

The pol gene encodes the reverse transcriptase. During the process of reverse transcription, the polymerase makes a double-stranded DNA copy of the dimer of single-stranded genomic RNA present in the virion. RNase H removes the original RNA template from the first DNA strand, allowing synthesis of the complementary strand of DNA. The predominant functional species of the polymerase is a heterodimer. All of the pol gene products can be found within the capsid of released virions.

The IN protein mediates the insertion of the proviral DNA into the genomic DNA of an infected cell. This process is mediated by three distinct functions of IN.

The Env protein is expressed from singly spliced mRNA. First synthesized in the endoplasmic reticulum, Env migrates through the Golgi complex where it undergoes glycosylation. Env glycosylation is generally required for infectivity. A cellular protease cleaves the protein into a transmembrane domain and a surface domain.

The viral genomic RNA expressed from some ERVs of a genome can be released from the cells in the form of VPs. Other expressed ERVs may cause the formation of VLPs such as RVLPs (retroviral like particles) but not of VPs, and thus may not lead to the release of particles containing a viral genomic RNA. However, generally the ones that are released have a higher potential to become infectious.

In the context of the present application VPs refer to viral particles that contain at least a part of a viral genome. In some instances, the VPs may comprise the full-length viral genomic RNA and thus may be functional VPs. VLPs as used in the context of the present invention are particles that appear to be VPs, but lack any part of the viral genome.

A vector according to the present invention is a nucleic acid molecule capable of transporting other nucleic acids to which it has been linked. A plasmid is, e.g., a type of vector. A viral vector is another type of vector, e.g., a lentivirus or an adeno-associated virus (AAV) vector.

In certain aspects of the present invention a vector is used to transport exogenous nucleic acids into a cell or cell population.

Exogenous nucleic acid as it is used herein means that the referenced nucleic acid is introduced into the host cell. The source of the exogenous nucleic acid may be, for example, a homologous or heterologous nucleic acid that expresses, e.g. a protein of interest. Correspondingly, the term endogenous refers to a nucleic acid molecule that is already present in the host cell. The term heterologous nucleic acid refers to a nucleic acid molecule derived from a source other than the species of the host cell, whereas homologous nucleic acid refers to a nucleic acid molecule derived from the same species as the host cell. Accordingly, an exogenous nucleic acid according to the invention can utilize either or both a heterologous and/or a homologous nucleic acid.

For example a cDNA of a human interferon gene is a heterologous exogenous nucleic acid when introduced in a CHO cell, but a homologous exogenous nucleic acid in a HeLa cell. The exogenous gene may be part of a vector when introduced into the cell or may be introduced without additional endogenous or exogenous nucleic acid sequences.

A transgene is an exogenous nucleic acid encoding a product such as a protein of interest, also referred to as “transgene expression product.” In certain embodiments more than one transgene is required to generate a cell line that produces a product of interest, in particular a protein of interest, e.g., an antibody, which might need a transgene that encodes the light chain and a transgene that encodes the heavy chain to produce the antibody, i.e., the protein of interest, as well as an antibiotic selection transgene used to select the stably transfected cells. A transgene expression product might also be just a marker protein such as an antibiotic selection gene, an Enhanced Green Fluorescent Protein (GFP) or β-galactosidase (lacZ). In this case the transgene may be integrated to be under the control of a specific gene promoter and may replace the completely or partially removed ERV or LTR-RT sequence or may be integrated into the allelic wild-type counterpart. Such a transgene can serve as a landing pad for integration of another transgene, such as a transgene encoding a protein of interest or a transgene expression product that together with another transgene expression product results in a protein of interest such as a therapeutic protein. For example, a transgene expression product may be a light chain or a heavy chain of an antibody, however the “protein of interest” is the immunoglobulin composed of 4 chains. However, generally, a product of interest, such as protein of interest, is a protein (including fusion protein) such as but not limited to a signaling protein such as α-IFN, β-IFN, γ-IFN, τ-IFN, ω-IFN, a cytokine such as erythropoietin or an antibody such as a monoclonal antibody, a fusion protein but also a regulatory RNA such as an siRNA or a shRNA or a mRNA. “Protein of interests” are the therapeutic proteins recovered from the cell supernatant and measured therein in picograms per cell and per day, μg/I or mg/I.

Transfection as used herein refers to the introduction of nucleic acids, including naked or purified nucleic acids or vectors carrying a specific nucleic acid into cells, in particular eukaryotic cells, including mammalian cells. Any know transfection method can be employed in the context of the present invention. Some of these methods include enhancing the permeability of a biological membrane to bring the nucleic acids into the cell. Prominent examples are electroporation or microporation. The methods may be used by themselves or can be supported by sonic, electromagnetic, and thermal energy, chemical permeation enhancers, pressure, and the like for selectively enhancing flux rate of nucleic acids into a host cell. Other transfection methods are also within the scope of the present invention, such as carrier-based transfection including lipofection or viruses (also referred to as transduction) and chemical based transfection. However, any method that brings a nucleic acid inside a cell can be used. A transiently-transfected cell will carry/express transfected RNA/DNA for a short amount of time and not pass it on. A stably-transfected cell will continuously express transfected DNA and pass it on: the exogenous nucleic acid has integrated into the genome of a cell.

“DNA Repair Pathway” or “DRP”, as used herein, refers to the cell mechanisms allowing a cell to maintain its genome integrity and its function, in response to the detection of DNA damages, such as single or double-strand breaks. Depending on several parameters such as the type and the length of DNA damages or the cell cycle phase in which the cell is at the moment of the said damages, DRPs refer to but are not limited to resection, canonical homology directed repair (canonical HDR), homologous recombination (HR), alternative homology directed repair (Alt-HDR), double-strand break repair (DSBR), single-strand annealing (SSA), synthesis-dependent strand annealing (SDSA), Break-induced replication (BIR), alternative end-joining (Alt-EJ), microhomology mediated end-joining (MMEJ), DNA synthesis-dependent microhomology-mediated end-joining (SD-MMEJ), non-homologous end joining (NHEJ) pathways such as canonical non-homologous end-joining (C-NHEJ) repair, alternative non-homologous end joining (A-NHEJ) pathway, translesion DNA synthesis (TLS) repair, base excision repair (BER), nucleotide excision repair (NER), mismatch repair (MMR), DNA damage responsive (DDR), Blunt End Joining, single strand break repair (SSBR), interstrand crosslink repair (ICL) and Fanconi Anemia pathway (FA). A DRP of the present invention is, however, preferably selected from the group enumerated above.

DNA repair pathways can be inhibited, or rather favored/enhanced. Genes, mRNA or corresponding proteins involved in such pathways can be modulated for inhibiting or favoring/enhancing a pathway (see examples in Table 2).

TABLE 2
DNA Repair Pathways and genes involved.

| DNA Repair pathway | Gene |
|---|---|
| resection, NHEJ, HR, MMEJ, SSA | Mre11 |
| resection, NHEJ, HR, MMEJ, SSA | Rad50 |
| resection, NHEJ, HR, MMEJ, SSA | Nbs1 |
| resection, HR, MMEJ, SSA | CtIP |
| resection, HR, NHEJ, FA | BRCA1 (FANCS) |
| resection, HR, NHEJ, MMEJ, SSA, MMR | Exo1 |
| Resection | RECQ1 |
| resection, HR, MMEJ, SSA | BLM |
| Resection | WRNa |
| Resection | RTSa |
| Resection | RECQ5 |
| Resection | Dna2 |
| Resection, NHEJ, HR | 53BP1 |
| Resection | EEPD1 |
| NHEJ | Xrcc4 |
| NHEJ | Ku70 |
| NHEJ | Ku80 |
| NHEJ, MMEJ | LigIV |
| NHEJ | DNA-PKcs |
| NHEJ, MMEJ | XRCC1 |
| NHEJ, MMEJ, BER | PARP1 |
| NHEJ | PARP2 |
| NHEJ | LigIII |
| NHEJ | Artemis |
| NHEJ | PNK |
| NHEJ | TDT |
| NHEJ | Pol μ (mu), POLM |
| NHEJ | Pol λ (lambda), POLL |
| NHEJ | XLF/Cernunnos |
| NHEJ | PAXX |
| NHEJ | TDP |
| NHEJ | APTX |
| NHEJ | WRN |
| NHEJ | RTEL1 |
| NHEJ | CYREN |
| NHEJ | APLF |
| HR | MDC1 |
| HR | Abraxas |
| HR, MMEJ | ATM |
| HR | Bard1 |
| HR, NHEJ | BRCA2 |
| HR | BRCC36 |
| HR | Cyclin D1 |
| HR | CK2alpha |
| HR | CK2beta |
| HR | DNA2 |
| HR | DNAPd |
| HR | DNAPh |
| HR | EME1 |
| HR, MMEJ, SSA, NER | ERCC1 |
| HR, NER, FA | ERCC4 (FANCQ) |
| HR, FA | FANCD1 |
| HR, FA | FANCD2 |
| HR | FANCF |
| HR | FANCM |
| HR | GEN1 |
| HR, NHEJ, MMEJ, SSA | MRE11 |
| HR | MUS81 |
| HR | Nbs1 |
| HR | H2AX |
| HR | Hop2 |
| HR | PALB2/FANCN |
| HR | PCNA |
| HR, FA | RAD51 (FANCR) |
| HR | RAD51AP1 |
| HR | Rad51B |
| HR, FA | Rad51C (FANCO) |
| HR | Rad51D |
| HR, SSA | RAD52 |
| HR | RAD54 |
| HR | XRCC2 |
| HR | XRCC3 |
| HR | RAP80 |
| HR | RMI1+ |
| HR | RMI2+ |
| HR | RNF168 |
| HR | RNF8 |
| HR | RPA1 |
| HR | RPA2 |
| HR | RPA3 |
| HR | GIY |
| HR | GIY-YIG |
| HR | SLX1 |
| HR | SLX4 (FANCP) |
| HR | SMC1 |
| HR | SMC3 |
| HR | SPO11 |
| HR | TIP60 |
| HR | TOPO II |
| HR | TOPOIII |
| HR | UBC13 |
| HR | WRN |
| HR | ChK1 |
| HR | ChK2 |
| HR | p53 |
| HR | CDC25 |
| HR, MMEJ, SSA | Srs2 |
| HR, MMEJ, SSA, NER | Xpf |
| HR, MMEJ | Pol δ (delta), Pol32 |
| HR | POLD1 |
| HR | POLD2 |
| HR | POLD3 |
| HR | POLD4 |
| HR | Pol ξ |
| HR | RecB |
| HR | RecC |
| HR | RecD |
| HR | HNRNPD |
| NHEJ | ETAA1 |
| HR | DSCC1 |
| NHEJ | CDK1 |
| NHEJ | CDK2 |
| HR | RBBP8 |
| NHEJ | ATR |
| NHEJ | PRKDC |
| HR, MMEJ, BER, NER, SSA | Ligase I |
| HR, MMEJ, BER, NER | Ligase III |
| MMEJ | Pol θ (theta), a.k.a. POLQ |
| MMEJ | Histone H1 |
| MMEJ | WRN |
| MMEJ, NHEJ | Pol β (beta), POLB |
| MMEJ, NHEJ | Pol4 |
| MMEJ, TLS | Pol η |
| MMEJ, TLS, HR | Pol ξ |
| MMEJ | PNK |
| SSA | RAD59 |
| SSA | XRS2 |
| SSA | Msh2 |
| SSA | Msh3 |
| SSA | Rad10 |
| SSA | DNA2 |
| SSA | RFC, RFC-like |
| SSA | PCNA-like protein |
| | (Rad1, Hus1, Rad9) |
| FA | FANCA |
| FA | FANCB |
| FA | FANCC |
| FA | FANCE |
| FA | FANCF |
| FA | FANCG |
| FA | FANCI |
| FA | FANCJ (BRIP1) |
| FA | FANCL |
| FA | FANCN |
| FA | FANCP |
| FA | FANCT |
| FA | FANCM |
| FA | FAAP100 |
| FA | FAAP24 |
| FA | FAAP20 |
| FA | FAAP16 |
| FA | FAAP10 |
| FA | BOD1L |
| FA | UHRF1 |
| FA | USP1 |
| FA | UAF1 |
| FA | AN1 |

Examples of NHEJ inhibitors (=inhibitors of PARP1, Ku70/80, DNA-PKcs, XRCC4/XLF, Ligase IV, Ligase III, XRCCI, Artemis, PNK) include without limitation, NU7441 (Leahy et al., Identification of a highly potent and selective DNA-dependent protein kinase (DNA-PK) inhibitor (NU7441) by screening of chromenone libraries. (Leahy et al., (2004), NU7026 (Willmore et al., 2004), Olaparib, DNA Ligase IV inhibitor, Scr7 (Maruyama et al., 2015)), KU-0060648 (Robert et al., 2015), anti-EGFR-antibody 0225 (Cetuximab) (Dittmann et al., 2005), Compound 401 (2-(4-Morpholinyl)-4H-pyrimido[2,I-a]isoquinolin-4-one), Vanillin, Wortmannin, DMNB, IC87361, LY294002, OK-1035, CO 15, NK314, PI 103 hydrochloride, to name just a few exemplary inhibitors.

MMEJ inhibitors, include, but are not limited to, MRE11 inhibitors such as Mirin and derivatives (Shibata et al, 2014), inhibitors of PoIQ, inhibitors of CtIP (Sfeir and Symington, 2015).

Examples of HR inhibitors include, but are not limited to RI-1 and BO2.

Examples of HR stimulators include, but are not limited to, RS-1 (RAD51 stimulator).

NHEJ stimulators, include, but are not limited to, IP6 (Inositol Hexakisphosphate, DNA-PK enhancer, Hanakahi 2000, Ma 2002, Cheung 2008).

A downmodulation of a DRP reduces the activity of such a DRP in a cell or population of cells. A downmodulation of a DRP can be by 10%, 20%, 30%, 40%, 50%, 60%, 70%, 80%, 90%, 95% or 100% of the repair activity (hereinafter “activity”) without the downmodulation. The downmodulation can be achieved in many ways, such as, but not limited to, contacting said cell or population of cells, with one or more inhibitor(s), such as a chemical inhibitor of the DRP/a component thereof, inactivating the DRP/a component thereof, downregulating the DRP/a component thereof (e.g. by contacting or expressing in said cell or population of cells one or more inhibitory nucleic acids such as a miRNA, a siRNA, a shRNA or any combination thereof) and/or mutating one or more genes of said DRP/a component thereof.

In a preferred embodiment a DRP is downmodulated that is either non-productive or competes with another DRP and is thus referred to as a competing pathway or non-productive pathway.

For example, a NHEJ pathway may be inhibited to favor productive integration of an exogenous DNA by e.g. MMEJ and related mechanisms. In the context of the present invention any active DRP may compete with another active DRP in a cell and is thus a competing DR pathway. A non-productive DRP in the context of the present invention is a pathway that will not or will only inefficiently mediate the integration of exogenous DNA into the cell genome. For example, synthesis-dependent strand annealing (SDSA), Break-induced replication (BIR), base excision repair (BER), nucleotide excision repair (NER), mismatch repair (MMR), DNA damage response (DDR), Blunt End Joining, single strand break repair (SSBR), and interstrand crosslink repair (ICL) are generally inefficient in mediating the integration of exogenous DNA.

The downmodulation of one DRP generally results in one or more other DNA repair pathways to take over the repair work of the downmodulated DRP. The one or more DRPs that take on the repair work is generally upmodulated. An upmodulation of the one or more DRPs can be by 10%, 20%, 30%, 40%, 50%, 60%, 70%, 80%, 90%, 95% or 100% of the activity without the downmodulation. A DRP that is upmodulated as a result of downmodulation of another competing DRP is considered “favored” (or enhanced) relative to the downmodulated DRP. The degree of favoring/enhancing may be proportional to the degree of downmodulation and may, e.g., be a 10%, 20%, 30%, 40%, 50%, 60%, 70%, 80%, 90% or 95% higher activity relative to the activity without the downmodulation of the downmodulated DRP. The activity of the downmodulated DRP may shift to one pathway, but may also shift to two or more pathways that take over the DNA repair functions of the downmodulated DRP.

Apart from downmodulating another DRP, a DRP may also be upmodulated, by, e.g., expressing, including causing the overexpression of, one or more components of said DRP in said cell or population of cells, introducing into said cell or population of cells, the component of the said DRP heterologously, or by contacting said cell, or population of cells, with one or more modulator, preferably a stimulator, such as a chemical stimulator of the one or more component of the said DRP, mutating one or more genes of said DRP, wherein said mutating enhances expression or activity of the one or more components of the said DRP.

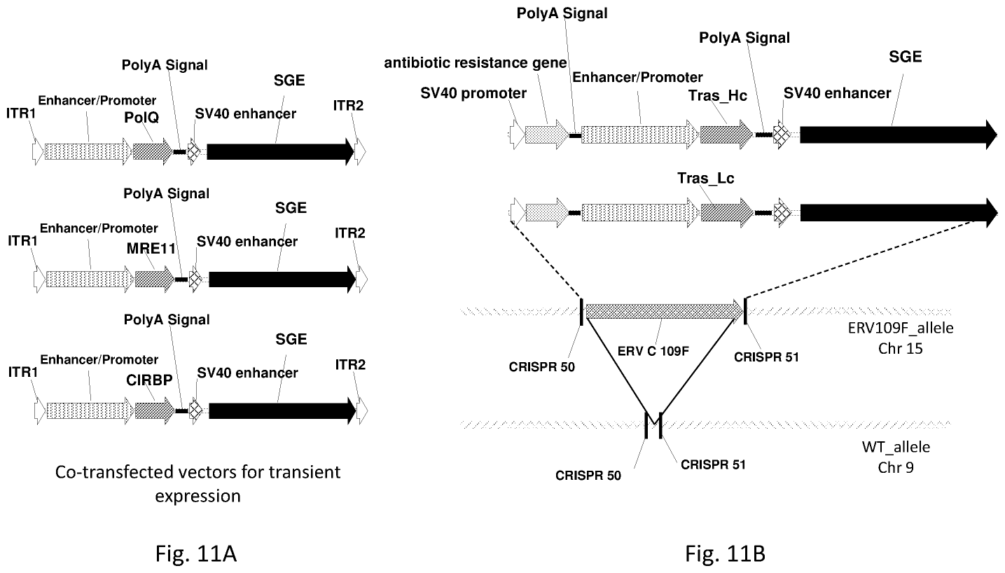

An modulation, in particular, an upmodulation can be achieved by, generally transiently, transfecting, e.g., co-transfecting (i.e. at the same time or within an hour), a cell/cell population with one or more vectors carrying one or more genes set forth in Table 2 and/or genes are listed under SEQ ID Nos: 25-28 and 38-59 and sequences having sequence identifies with these sequences as described elsewhere herein (e.g., 99%, 98%, 97%, 96% or 95%), together with or less than 24, 18, 12, 8, 4, 2, 1 hour before and in certain embodiments after transfecting the cell with the integrating vector(s) shown in FIG. 11B. In FIG. 11A some representative vectors are shown. However, as the person skilled in the art will appreciate, any gene encoding a protein involved in the DRP can be used in such a vector. Some of these genes are listed in Table 2 and certain preferred genes are listed under SEQ ID Nos: 25-28 and 38-59. As can be seen in FIG. 11A certain preferred vectors insert the DRP gene between ITRs (inverted terminal repeats), i.e. the DRP gene is “flanked” by two ITRs, and may, in certain preferred embodiments also include a genetic element such as a MAR (e.g., SGE). A transfection with one or more vectors carrying the MRE11, POLQ, CIRBP, and/or RAD51 is preferred, e.g., with one or more vectors carrying SEQ ID NO: 25 (hMRE11), SEQ ID NO: 26 (cgPOLQ), SEQ ID NO:27 (hCIRBP) and/or SEQ ID NO: 28 (hRAD51). Other preferred DRP genes are those whose expression products is/are used by: (i) both MMEJ and HR (Mre11, Rad50, Nbs1, CtIP, Exo1, BLM, ATM, ERCC1, Srs2, Xpf, Pol δ, Ligase I, ligand III), (ii) by MMEJ but not HR (e.g., LigIV, XRCC1, PARP1, POLQ, WRN, POLB, Pol4), and (iii) by HR but jot MMEJ proteins (e.g. BRCA1, 53BP1, MDC1).

A chemical stimulator, as used herein, refers to a chemical compound that can be used to enhance the expression of a gene or the activity of a protein. As the person skilled in the art will readily recognize, the chemical stimulator will depend which component of which DPR (DNA Repair Pathway) is stimulated. For example, RS-1, a RAD51 stimulator stimulates HR. IP6 (Inositol Hexakisphosphate), and other DNA-PK enhancers are NHEJ stimulators (see, e.g., Hanakahi 2000, Ma 2002, Cheung 2008).

A chemical inhibitor, as used herein, refers to a chemical compound that can be used to inhibit the expression of a gene or the activity of a protein. As the person skilled in the art will also readily recognize, the chemical inhibitor will depend which component of which DPR is stimulated. Examples of chemical inhibitors of MMEJ include, but are not limited to MRE11 inhibitors such as Mirin and derivatives (Shibata et al, Molec. Cell (2014) 53:7-18), inhibitors of PoIQ, inhibitors of CtIP (Sfeir and Symington, “Microhomology-Mediated End Joining: A Back-up Survival Mechanism or Dedicated Pathway?” Trends Biochem Sci (2015) 40:701-714). Examples of HR inhibitors: RI-1 (RAD51 Inhibitor 1) and BO2 (3-(Phenylmethyl)-2-[(1E)-2-(3-pyridinyl)ethenyl]-4(3H)-quinazolinone). See also US Patent Pubs. 2019/0194694A1 and 2015/0361451 A1.

Chemical stimulators and inhibitors are generally exogenous, i.e., added to the cell supernatant and taken up by the cell. Such inhibitors maybe added to the cells/cell populations with the various vectors described herein, e.g., at the same time or within an hour or within less than 24, 18, 12, 8, 4, 2 hours.

Nucleases and/or Nickases: Double/Single Strand Breaks Introduction

Different molecules are able to introduce double and/or single strand breaks into genomic nucleic acids. The nucleases or nickases of the present invention include, but not limited to, homing endonucleases, restriction enzymes, zinc-finger nucleases or zinc-finger nickases, meganucleases or meganickases, transcription activator-like effector (TALE) nucleases or TALE nickases, guided, in particular nucleic acid guided nucleases or nickases, such as a RNA-guided nucleases or RNA-guided nickases, DNA-guided nucleases, such as the Argonaute (NgAgo) of Natronobacterium gregoryi or DNA-guided nickases, a megaTAL nuclease, a BurrH-nuclease, ARCUS nucleases, a modified or chimeric version or variant thereof, and combinations thereof. The RNA-guided nuclease or the RNA-guided nickase are optionally part of a CRISPR-based system.

In a preferred embodiment, these double and/or single strand breaks are introduced by one or more nucleases or nickase. Nucleases can introduce double and/or single strand breaks. The term nickase is reserved to molecules that introduce single strand breaks and may be a nuclease with a partially inactive DNA cleavage domain. For example, nuclease domains of the nucleases may be mutated independently of each other to create DNA “nickases” capable of introducing a single-strand cut with the same specificity as the respective nuclease. With the limitations mentioned herein the following discussions about nucleases equally apply to nickases.

Nucleases are capable of cleaving phosphodiester bonds between monomers of nucleic acids. Many nucleases participate in DNA repair by recognizing damage sites and cleaving them from the surrounding DNA. These enzymes may be part of complexes. Exonucleases are nucleases that digest nucleic acids from the ends. Endonucleases, which are preferred in the present context, are nucleases that act on central regions of the target molecules. Deoxyribonuclease act on DNAs and ribonucleases act on RNA. Many nucleases involved in DNA repair are not sequence-specific. In the present context, however, sequence-specific nucleases are preferred. In one preferred embodiment, sequence-specific nuclease(s) is/are specific for fairly large stings of nucleotides in the target genome, such as 5 and more nucleotides, or 10, 15, 20, 25, 30, 35, 40, 45 or even 50 or more nucleotides, the ranges of 5-50, 10-50, 15-50, 15-40, 15-30 as target sequences in the target genome are preferred in certain embodiments. The larger such a “recognition sequence” the fewer target sites are in a genome and the more specific the cut the nucleases or nickases make into the genome is, ergo the cuts become site specific. A site-specific nuclease has generally less than 10, 5, 4, 3, 2 or just a single (1) target site in a genome. Nucleases that have been engineered for altering genomic nucleic acid(s), including by cutting specific genomic target sequences, are referred to herein as engineered nucleases. CRISPR-based systems are one type of engineered nuclease(s). However, such an engineered nuclease can be based on any nuclease described herein. In one preferred embodiment, the codon(s) of the respective nuclease(s) are optimized for expression in, eukaryotic cells, e.g., mammalian cells. The nucleases/systems of the present invention may also comprise one or more linkers and/or additional functional domains, e.g. an end-processing enzymatic domain of an end-processing enzyme that exhibits 5-3′ exonuclease or 3-5′ exonuclease or other non-nuclease domains, e.g. a helicase domain.

Restriction enzymes are sequence specific nucleases that often are specific for fairly small strings of nucleotides, ergo that have a short recognition sequence. The first letter of the name comes from the genus and the second two letters come from the species of the prokaryotic cell from which they were isolated. For example, EcoRI stems from Escherichia coli RY13 bacteria. Many restriction enzymes are restriction endonucleases and introduce, e.g., a blunt or staggered cut(s), into the middle of a nucleic acid. Many restriction enzymes are sensitive to the methylation states of the DNA they target. Cleavage may be blocked, or impaired, when a particular base in the enzyme's recognition site is modified.

Examples of methylation-sensitive restriction enzymes important in epigenetics include, DpnI and DpnII which are sensitive for N6-methyladenine detection within GATC recognition site and HpaII and MspI which are sensitive for C5-methylcytosine detection within CCGG recognition site.

Some exemplary restriction enzymes used in the examples are listed In Table 3: together with their recognition site, their CpG methylation sensitivity and the number of target sites found in the CHO genome of reference.

TABLE 3
Examples of Restriction Enzymes and their target sites in the CHO genome.

| | Recognition | | | |
|---|---|---|---|---|
| | sequence in CHO | | CpG Methylation | Number of target |
| Enzyme | genome | SEQ ID NOS: | sensitivity | sites |
| Pvul | 5′ . . . CG AT [symbol] CG . . . 3′ | 61 | Blocked | 11′605 |
| | 3′ . . . GC [symbol] TA GC . . . 5′ | | | |
| Sbfl | 5′ . . . CC | 62 | — | 70′162 |
| | TGCA [symbol] GG . . . 3′ | | | |
| | 3′ . . . GG [symbol] ACGT | | | |
| | CC . . . 5′ | | | |
| Ascl | 5′ . . . GG [symbol] CGCG | 63 | Blocked | 3′901 |
| | CC . . . 3′ | | | |
| | 3′ . . . CC | | | |
| | GCGC [symbol] GG . . . 5′ | | | |
| BstBl | 5′ . . . TT [symbol] CG AA . . . 3′ | 64 | Blocked | 105′498 |
| | 3′ . . . AA GC [symbol] TT . . . 5′ | | | |

Endonucleases recognizing sequences larger than 12 base pairs are called meganucleases. Meganucleases/-nickases are endodeoxyribonucleases characterized by a large recognition site (double-stranded DNA sequences of, e.g., 12 to 40 base pairs, such as 20-40 or 30-40 base pairs); as a result, this site might only occur once in any given genome.

“Homing endonuclease” are a form of meganucleases and are double stranded DNases that have large, asymmetric recognition sites and coding sequences that are usually embedded in either introns or inteins. Homing endonuclease recognition sites are extremely rare within the genome so that they cut at very few locations, sometimes a singular location within in the genome (WO2004067736, see also U.S. Pat. No. 8,697,395 B2).

Zinc-finger nucleases/-nickases (ZFNs) are artificial restriction enzymes generated by fusing zinc finger DNA-binding domains to a DNA-cleavage domain. Zinc finger domains can be engineered to target specific desired DNA sequences. ZFNs as described, for instance, by Urnov F., et al. (Highly efficient endogenous human gene correction using designed zinc-finger nucleases (2005) Nature 435:646-651)

Transcription activator-like effector (TALE) nucleases/-nickases are restriction enzymes that can be engineered to cut specific sequences of DNA. Transcription activator-like effectors (TALEs) can be engineered to bind to practically any desired DNA sequence, so when combined with a DNA-cleavage domain, DNA can be cut at specific locations. TALE-Nuclease as described, for instance, by Mussolino et a/. (A novel TALE nuclease scaffold enables high genome editing activity in combination with low toxicity (2011) Nucl. Acids Res. 39(21):9283-9293).

RNA-guided nucleases/-nickases, in particular endonucleases include, for example Cas9 or Cpf1. The CRISPR system has been described in detail. Any CRISPR based system is part of the present invention. In case another RNA-guided endonuclease(s) is/are used, an appropriate guide-RNA, sgRNA or crRNA or other suitable RNA sequences that interacts with the RNA-guided endonuclease and targets to a genomic target site in the genomic nucleic acid can be used.

In certain preferred embodiments, the nuclease is a RNA-guided nuclease. Non-limiting examples of RNA-guided nucleases, including nucleic acid-guided nucleases, for use in the present disclosure include, but are not limited to, CasI, CasIB, Cas2, Cas3, Cas4, Cas5, Cas6, Cas7, Cas8, Cas9 (also known as CsnI and CsxI2), Cas10, CasX, CasY, Cpf1, CsyI, Csy2, Csy3, CseI, Cse2, CscI, Csc2, Csa5, Csn2, Csm2, Csm3, Csm4, Csm5, Csm6, CmrI, Cmr3, Cmr4, Cmr5, Cmr6, CsbI, Csb2, Csb3, CsxI7, CsxI4, CsxIO, CsxI6, CsaX, Csx3, CsxI, CsxI5, CsfI, Csf2, Csf3, Csf4, Cms1, Cpf1, homologues thereof, orthologues thereof, or modified versions thereof, MAD7 such as MADzymes (INSCRIPTA), C2cI, C2c2, C2c3.

In certain preferred embodiments, the nuclease is a DNA-guided nuclease. An “DNA-guided nuclease” refers to a system comprising a DNA guide (gDNA) and an endonuclease. The DNA guide, such as a 5′-phosphorylated single-stranded DNA (ssDNA) guides endonuclease to cleave double-stranded DNA targets within DNA-guided nickase. An “Argonaute-based system” refers to a DNA-guided nuclease based on a single-stranded DNA guide (gDNA) and an endonuclease from the Argonaute (Ago) protein family. The gDNA targets the endonuclease to a specific DNA sequence resulting in sequence-specific DNA cleavage. Ago proteins can be altered via mutagenesis to have improved activity at 37° C. Several Argonaute proteins were characterized from Natronobacterium gregoryi (NgAgo, see, e.g., Gao et al., DNA-guided genome editing using the Natronobacterium gregoryi Argonaute, Nature Biotechnology, published online May 2, 2016), Rhodobacter sphaeroides (RsAgo, see, e.g., Olivnikov et al.), Thermo thermophiles (TtAgo, se e.g. Swarts et al (2014), Nature 507(7491): 258-261), Pyrococcus furiosus Argonaute (PfAgo).

The use of an Argonaute-based system allows for targeted cleavage of genomic DNA within cells.

“TtAgo” is a prokaryotic Argonaute protein thought to be involved in gene silencing. TtAgo is derived from the bacteria Thermus thermophilus. (See, e.g., Swarts et al, ibid, G. Sheng et al, (2013) Proc. Natl. Acad. Sci. U.S.A. III, 652).

One of the most well-known prokaryotic Ago protein is the one from T. thermophilus (TtAgo; Swarts et al. ibid). This “guide DNA” bound by TtAgo serves to direct the protein-DNA complex to bind a Watson-Crick complementary DNA sequence in a third-party molecule of DNA. Once the sequence information in these guide DNAs has allowed identification of the target DNA, the TtAgo-guide DNA complex cleaves the target DNA. Such a mechanism is also supported by the structure of the TtAgo-guide DNA complex while bound to its target DNA (G. Sheng et al, ibid). Ago from Rhodobacter sphaeroides (RsAgo) has similar properties (ibid).

Exogenous guide DNAs of arbitrary DNA sequences can be loaded onto the TtAgo protein (Swarts et al. ibid.). Since the specificity of TtAgo cleavage is directed by the guide DNA, a TtAgo-DNA complex formed with an exogenous, investigator-specified guide DNA will therefore direct TtAgo target DNA cleavage to a complementary investigator-specified target DNA. In this way, one may create a targeted double-strand break in DNA. Use of the TtAgo-guide DNA system (or orthologous Ago-guide DNA systems from other organisms) allows for targeted cleavage of genomic DNA within cells. Such cleavage can be either single- or double-stranded. For cleavage of mammalian genomic DNA, it would be preferable to use of a version of TtAgo codon optimized for expression in mammalian cells. Further, it might be preferable to treat cells with a TtAgo-DNA complex formed in vitro where the TtAgo protein is fused to a cell-penetrating peptide. Ago-RNA-mediated DNA cleavage could be used to effect a panoply of outcomes including gene knock-out, targeted gene addition, gene correction, targeted gene deletion using techniques standard in the art for exploitation of DNA breaks.

Illustrative examples of Argonaute-based systems and design of gDNAs are disclosed in WO 2017/107898, CN105483118, WO 2017/139264, U.S. Patent Application Nos. 2017367280 and 20180201921, and references cited therein, all of which are incorporated herein by reference in their entireties. An Argonaute-based system optionally comprises one or more linkers and/or additional functional domains, e.g. an end-processing enzymatic domain of an end-processing enzyme that exhibits 5-3′ exonuclease or 3-5′ exonuclease or other non-nuclease domains, e.g. a helicase domain.

A “megaTAL nuclease/-nickase” refers to an engineered nuclease comprising an engineered TALE DNA-binding domain and an engineered meganuclease or an engineered homing endonuclease. TALE DNA-binding domains can be designed for binding DNA at almost any locus of a nucleic acid sequence in a genome, and cleave the target sequence if such a DNA-binding domain is fused to an engineered meganuclease. Illustrative examples of megaTAL nuclease and design of TALE DNA-binding domains are disclosed in described, for instance by Boissel et al. (MegaTALs: a rare-cleaving nuclease architecture for therapeutic genome engineering (2013), Nucleic Acids Research 42 (4):2591-2601), and references cited therein, all of which are incorporated herein by reference in their entireties. A megaTAL nuclease optionally comprises one or more linkers and/or additional functional domains, e.g. a C-terminal domain (CTD) polypeptide, a N-terminal domain (NTD) polypeptide, an end-processing enzymatic domain of an end-processing enzyme that exhibits 5-3′ exonuclease or 3-5′ exonuclease, or other non-nuclease domains, e.g. a helicase domain.

A “TALE DNA binding domain” is the DNA binding portion of transcription activator-like effectors (TALE or TAL-effectors), which mimics plant transcriptional activators to manipulate the plant transcriptome (see e.g., Kay et al., 2007. Science 318:648-651). TALE DNA binding domains contemplated in particular embodiments are engineered de novo or from naturally occurring TALEs, and include, but are not limited to, AvrBs3 from Xanthomonas campestris pv. vesicatoria, Xanthomonas gardneri, Xanthomonas translucens, Xanthomonas axonopodis, Xanthomonas perforans, Xanthomonas alfalfa, Xanthomonas citri, Xanthomonas euvesicatoria, and Xanthomonas oryzae and brgI 1 and hpxI7 from Ralstonia solanacearum. Illustrative examples of TALE proteins for deriving and designing DNA binding domains are disclosed in U.S. Pat. No. 9,017,967, and references cited therein, all of which are incorporated herein by reference in their entireties.

A “BurrH-nuclease” refers to a fusion protein having nuclease activity, that comprises modular base-per-base specific nucleic acid binding domains (MBBBD). These domains are derived from proteins from the bacterial intracellular symbiont Burkholderia Rhizoxinica or from other similar proteins identified from marine organisms. By combining together different modules of these binding domains, modular base-per-base binding domains can be engineered for having binding properties to specific nucleic acid sequences, such as DNA-binding domains. Such engineered MBBBD can thereby be fused to a nuclease catalytic domain to cleave DNA at almost any locus of a nucleic acid sequence in a genome. Illustrative examples of BurrH-nucleases and design of MBBBDs are disclosed in WO 2014/018601 and US2015225465 A1, and references cited therein, all of which are incorporated herein by reference in their entireties. A BurrH-nuclease optionally comprises one or more linkers and/or additional functional domains, e.g. an end-processing enzymatic domain of an end-processing enzyme that exhibits 5-3′ exonuclease or 3-5′ exonuclease or other non-nuclease domains, e.g. a helicase domain.

Enzymes such as transposases or integrases may also be used as nickases/nucleases in the context of the disclosed methods and cells.

Targeting elements for targeting at least one locus of the genome of a cell comprising an insertion site of an endogenous retrovirus (ERV) sequence or a LTR-retrotransposon (LTR-RT) sequence are generally sequences that facilitate and/or guide the activity of the nickase and/or nuclease. Such targeting elements comprise, e.g., guide RNA, including single guide RNA (sgRNA) or crRNA (CRISPR RNA) and are encoded by CRISPR and e.g. Cas9, Cpf1 or Cms1 nuclease expression vectors targeting the ERV C 109F 5′ genomic sequences (SEQ ID 8 and SEQ ID 10), and the ERV C 109F 3′ genomic sequences (SEQ ID 9 and SEQ ID 11). The DRP that may be upregulated and/or downregulated may be adjusted depending on the type of element used. For example, for DSBs created by CRISPR cleavage site 16 and CRISPR cleavage site 17 (see FIGS. 5A and 5B), a homologous recombination (HR) pathway is in certain embodiments upregulated. In addition, in particular a vector carrying the transgene might include 5′ and 3′ homology arms (SEQ ID 6 and SEQ ID 7), which are present in the vector and the locus, including the insertion site.

The sequence specificity of CRISPR (clustered, regularly interspaced, short palindromic repeats) systems is determined by small RNAs. CRISPR loci are composed of a series of repeats separated by ‘spacer’ sequences that match the genomes of bacteriophages and other mobile genetic elements. The repeat-spacer array is transcribed as a long precursor and processed within repeat sequences to generate small crRNA that specify the target sequences (also known as protospacers) cleaved by CRISPR systems. For cleavage, the presence of a sequence motif immediately downstream of the target region is often required, known as the protospacer-adjacent motif (PAM). CRISPR-associated (cas) genes usually flank the repeat-spacer array and encode the enzymatic machinery responsible for crRNA (CRISPR RNA) biogenesis and targeting. For instance, Cas9 is a dsDNA endonuclease that uses a crRNA guide to specify the site of cleavage. Loading of the crRNA guide onto Cas9 occurs during the processing of the crRNA precursor and requires a small RNA antisense to the precursor, the tracrRNA, and RNAse Ill. In contrast to genome editing with ZFNs or TALENs, changing Cas9 target specificity does not require protein engineering but only the design of the short crRNA guide, also termed sgRNA when crRNA is fused to tracrRNA (trans-activating CRISPR RNA).

To date, three different types of the Cas9 nuclease (e.g. Cas 9) have been adopted in genome-editing protocols. The first is wild-type Cas9, which can site-specifically cleave double-stranded DNA, resulting in the activation of the double strand break (DSB) repair machinery. DSBs can be repaired by the cellular Non-Homologous End Joining (NHEJ) pathway, resulting in insertions and/or deletions (indels) which disrupt the targeted locus. Alternatively, if a donor template with homology to the targeted locus is supplied, the DSB may be repaired by the homology-directed repair (HDR) pathway allowing for precise replacement mutations to be made.

The Cas9 system was further engineered towards increased precision by developing a mutant form, known as nCas9, with only nickase activity (e.g. Cas9D10A). This means it cleaves only one DNA strand, and does not activate NHEJ. Instead, when provided with a homologous repair template, DNA repairs are conducted via the high-fidelity HDR pathway only, resulting in reduced indel mutations. Cas9D10A is therefore in many applications more appealing in terms of target specificity when loci are targeted by paired Cas9 complexes designed to generate adjacent DNA nicks. Such Cas nickase can also be fused to other functional or catalytic domain, such as a domain providing a deamination activity (e.g. for base editing purposes).

The third type is based on an enzymatically inactive Cas9 (eiCas9), also known as Cas9 endonuclease Dead (dead Cas9 or dCas9). This system comprises a Cas9 mutant that lacks endonuclease activity due to mutations in its endonuclease domains (e.g. RuvC and HNH domains). dCas9 it is still capable of binding to its guide RNA and the DNA strand and can be fused a functional or catalytic domain such as a domain providing a DNA-modifying activity selected from but are not limited to nuclease activity (e.g. Fok1), Clo51, methyltransferase activity, demethylase activity, deamination activity, depurination activity, integrase activity, transposase activity, and recombinase activity. Other domains providing a protein modifying activity include but are not limited to repression domains (e.g. KRAB domain), activation domains (e.g. VP16), methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, demyristoylation activity, glycosylation activity and deglycosylation activity.

The term sequence identity refers to a measure of the identity of nucleotide sequences or amino acid sequences. In general, the sequences are aligned so that the highest order match is obtained. “Identity”, per se, has a recognized meaning in the art and can be calculated using published techniques. (See, e.g.: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991). While there exist a number of methods to measure identity between two polynucleotide or polypeptide sequences, the term “identity” is well known to skilled artisans as defining identical nucleotides or amino acids at a given position in the sequence (Carillo, H. & Lipton, D., SIAM J Applied Math 48:1073 (1988)).

Whether any particular nucleic acid molecule is at least 50%, 60%, 70%, 75%, 80%, 85%, 90%, 95%, 96%, 97%, 98% or 99% identical to, for instance, the gammaretrovirus-like sequences of SEQ ID NOs. 1, 2, 3, 4, 5 or a part thereof can be determined conventionally using known computer programs such as DNAsis software (Hitachi Software, San Bruno, Calif.) for initial sequence alignment followed by ESEE version 3.0 DNA/protein sequence software for multiple sequence alignments.

Whether the amino acid sequence is at least 50%, 60%, 70%, 75%, 80%, 85%, 90%, 95%, 96%, 97%, 98% or 99% identical to, for instance a protein expressed by SEQ ID NOs: 1 or 3 or a part thereof, can be determined conventionally using known computer programs such the BESTFIT program (Wisconsin Sequence Analysis Package®, Version 8 for Unix, Genetics Computer Group, University Research Park, 575 Science Drive, Madison, Wis. 53711). BESTFIT uses the local homology algorithm of Smith and Waterman, Advances in Applied Mathematics 2:482-489 (1981), to find the best segment of homology between two sequences.

When using DNAsis, ESEE, BESTFIT or any other sequence alignment program to determine whether a particular sequence is, for instance, 95% identical to a reference sequence according to the present invention, the parameters are set such that the percentage of identity is calculated over the full length of the reference nucleic acid or amino acid sequence and that gaps in homology of up to 5% of the total number of nucleotides in the reference sequence are allowed.

Another preferred method for determining the best overall match between a query sequence (a sequence of the present invention) and a subject sequence, also referred to as a global sequence alignment, can be determined using the FASTDB computer program based on the algorithm of Brutlag et al. (Comp. App. Biosci. (1990) 6:237-245). In a sequence alignment the query and subject sequences are both DNA sequences. An RNA sequence can be compared by converting U's to T's. The result of said global sequence alignment is in percent identity. Preferred parameters used in a FASTDB alignment of DNA sequences to calculate percent identity are: Matrix=Unitary, k-tuple=4, Mismatch Penalty=1, Joining Penalty=30, Randomization Group Length=0, Cutoff Score=1, Gap Penalty=5, Gap Size Penalty=0.05, Window Size=500 or the length of the subject nucleotide sequence, whichever is shorter.

For example, a polynucleotide having 95% “identity” to a reference nucleotide sequence of the present invention, is identical to the reference sequence except that the polynucleotide sequence may include on average up to five point mutations per each 100 nucleotides of the reference nucleotide sequence encoding the polypeptide. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. The query sequence may be an entire sequence, the ORF (open reading frame), or any fragment specified as described herein.

The NCBI Basic Local Alignment Search Tool (BLAST) (Altschul et al. J. Mol. Biol. 215:403-410, 1990) is available from several sources, including the National Center for Biotechnology Information (NCBI, Bethesda, Md.) and on the Internet, for use in connection with the sequence analysis programs blastp, blastn, blastx, tblastn and tblastx. It can be accessed at the NCBI website, together with a description of how to determine sequence identity and sequence similarities using this program.

The invention is not only directly to sequences having a certain sequence identity with the sequences disclosed herein but is, equally, directed to sequence variants of any of the sequences disclosed herein. The invention is thus also directed to sequence variants in any context in which a certain sequence identity is mentioned and vice versa. A “sequence variant” refers to a polynucleotide or polypeptide differing from the sequences disclosed herein (polynucleotide or polypeptide sequences), but retaining essential properties thereof. Generally, variants are closely similar and in many regions, identical to the sequences herein disclosed.

The variants may contain alterations in the coding regions, non-coding regions, or both. Especially preferred are sequence variants containing alterations which produce silent substitutions, additions, or deletions, but do not alter the properties or activities of, e.g., the encoded polypeptide. Nucleotide variants produced by silent substitutions due to the degeneracy of the genetic code are preferred. Moreover, variants in which 5-10, 1-5, or 1-2 amino acids are substituted, deleted, or added in any combination are also preferred.

The amino acid sequences of the variant polypeptides may differ from the amino acid sequences depicted in SEQ ID NOS: 3 by an insertion or deletion of one or more amino acid residues and/or the substitution of one or more amino acid residues by different amino acid residues. Preferably, amino acid changes are of a minor nature, that is conservative amino acid substitutions that do not significantly affect, e.g., the folding and/or activity of the protein; small deletions, typically of one to about 30 amino acids; small amino- or carboxyl-terminal extensions, such as an amino-terminal methionine residue; a small linker peptide of up to about 20-25 residues; or a small extension that facilitates purification by changing net charge or another function, such as a poly-histidine tract, an antigenic epitope or a binding domain. Examples of conservative substitutions are within the group of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions which do not generally alter the specific activity are known in the art and are described, for example, by H. Neurath and R. L. Hill, 1979, In, The Proteins, Academic Press, New York. The most commonly occurring exchanges are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/IIe, Leu/Val, as well as these in reverse. A certain percentile of “consecutive nucleotides” means nucleotides directly following each other. Thus 10% of the nucleotides of SEQ ID NO:2, which contains 60000 nucleotides could be nucleotide 1-6000 or nucleotide 2-6001 etc.

Gene silencing via, e.g., siRNAs has been described elsewhere, for example in US Patent Publication 20180016583, which is incorporated herein by reference in its entirety, and specifically for its disclosure and gene silencing.

### EXAMPLES

The aim of the following examples is to first confirm transgene insertion into the locus, here via

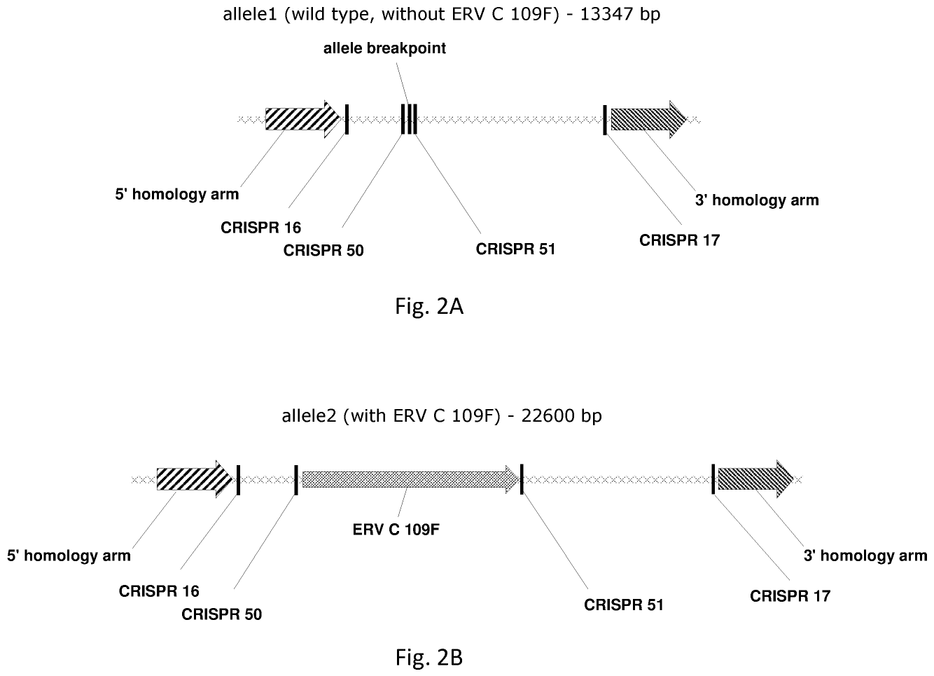

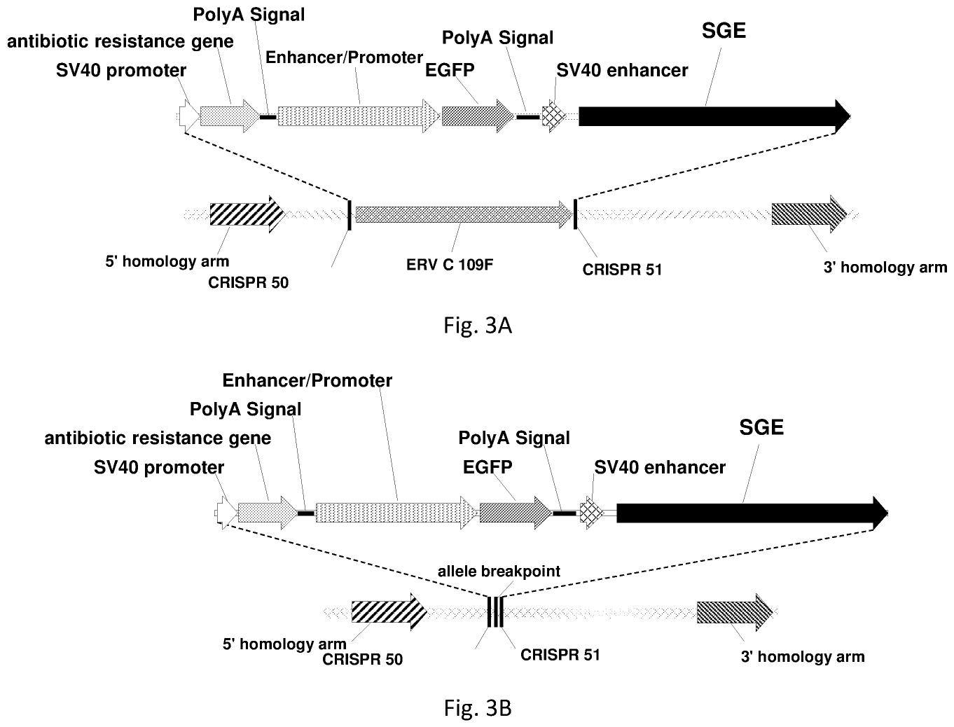

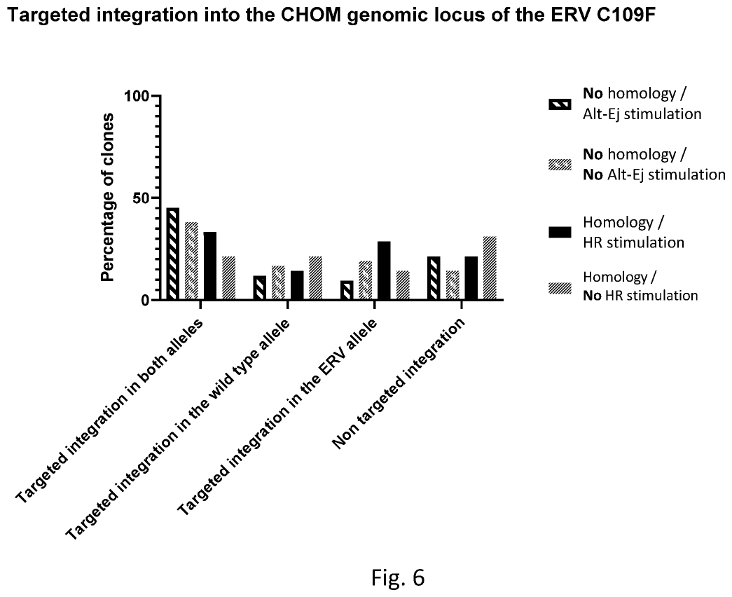

CRISPR-mediated cleavage (FIG. 2) and secondly, to compare the effect of homology on the transgene sequence with the integration locus versus no homologies on the transgene (FIGS. 3A, 3B, 4A, 4B, 5A, 5B). In addition, the effect of the up-/downregulation, stimulation/inhibition of DNA repair pathways such as homologous recombination (HR) or Alternative end-joining (Alt-EJ) was compared (FIG. 6).

TABLE 4
Summary of the experiments.

| | | | Upmodulated/Stimulated | | Downmodulated/Inhibited | |
|---|---|---|---|---|---|---|
| | | | DNA | | DNA | |
| | | | recombination pathway | | recombination pathway | |
| | | | DNA | | DNA | |
| | | | Repair | upregulated | Repair | downregulated |
| Pool | DNA vector | CRISPR | Pathway | genes | Pathway | genes |
| 1 | Vector | Cleavage | Alt-EJ | hMRE11 | NHEJ | DNA-PK |
| | without | site 50-51 | | (overexpression) | | (chemical |
| | CHO-M ERV | close to ERV | | cgPOLQ | | inhibition with |
| | homologies | C109F | | (overexpression) | | NU7441) |
| | | | | CIRBP | | |
| | | | | (overexpression) | | |
| 2 | | | X | X | NHEJ | DNA-PK |
| | | | | | | (chemical |
| | | | | | | inhibition with |
| | | | | | | NU7441) |
| 3 | Vector with | Cleavage | HR | hMRE11 | NHEJ | DNA-PK |
| | CHO-M | site 16-17 | | (overexpression) | | (chemical |
| | homologies | close to | | hRAD51 | | inhibition with |
| | arms | CHO-M ERV | | (overexpression + | | NU7441) |
| | | homologies | | chemical | | |
| | | | | stimulation with | | |
| | | | | RS-1) | | |
| 4 | | | X | X | NHEJ | DNA-PK |
| | | | | | | (chemical |
| | | | | | | inhibition with |
| | | | | | | NU7441) |

Pool 1: No DNA homology/Alt-EJ stimulation/NHEJ inhibition
Pool 2: No DNA homology/No Alt-EJ stimulation/NHEJ inhibition
Pool 3: DNA homology/HR stimulation/NHEJ inhibition
Pool 4: DNA homology/No HR stimulation/NHEJ inhibition

### Example 1: Targeted Integration into the ERV C 109F Locus

This example illustrates the transgene integration into the ERV C 109F locus (SEQ ID NO: 1, 2).

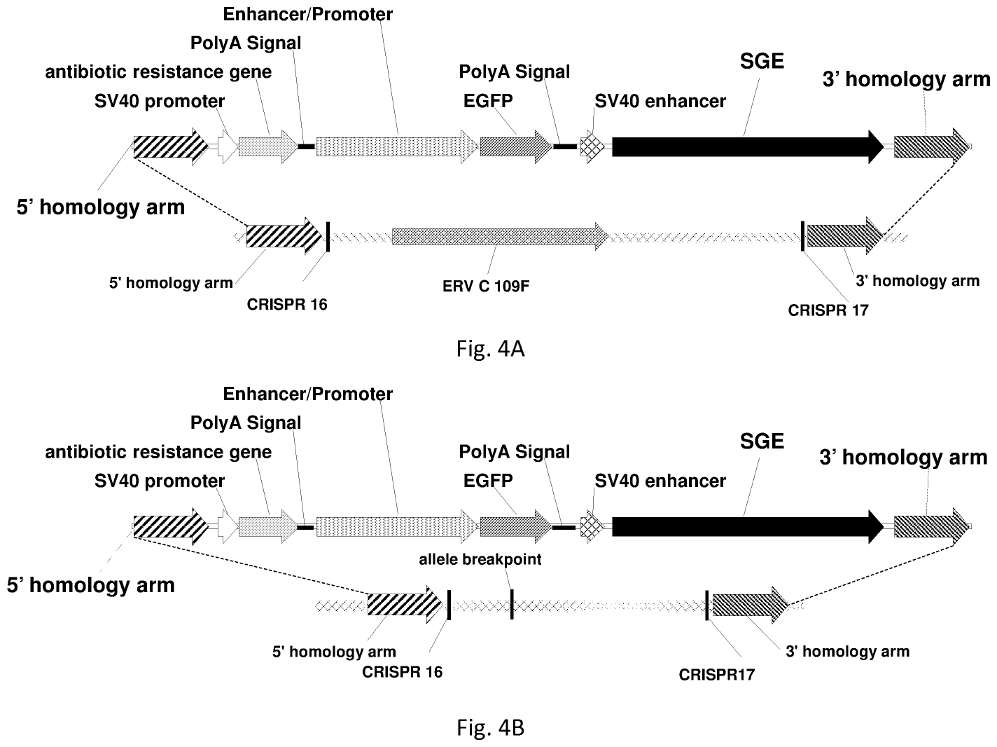

CHO-M cells were transfected with vectors (FIGS. 3A, 3B, 4A, 4B), targeting each of the genomic ERV C 109F loci. Such vectors either comprise no homology sequences (FIG. 3A, 3B) or do comprise homology sequences (FIGS. 4A, 4B). For both the “no homology” and the “homology” approach, the non-homologous end joining (NHEJ) DNA Repair Pathways was inhibited using the chemical inhibitor Nu7441. Moreover, each approach included experiments directed at the evaluation of the presence or absence of stimulation of homologous recombination (HR) or alternative end-joining (Alt-EJ).

For both approaches, there were four possibilities of transgene integrations in the defined ERV C 109F locus: i) no transgene integration, or ii) integration in the WT allele of the ERV C 109F locus, or iii) integration in the allele ERV-C 109F, or finally iv) integration in both alleles (ii) and (iii), sometime referred to herein also as “both loci.”

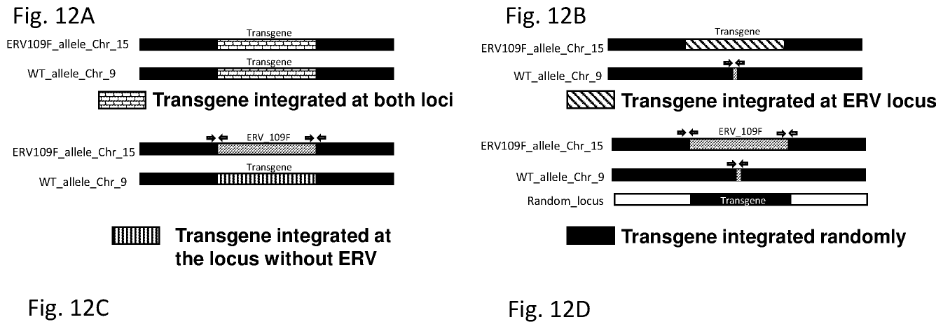

These results were obtained using 3 TaqMan qPCR assays developed in order to determine the category of each clone. These assays are explained in the context of FIGS. 5A and 5B, and FIG. 12.

FIG. 6 shows the percentage of clones that fell into the categories (i), (ii), (iii) and (iv) as defined above. The Figure shows in particular the targeting efficiency of CRISPR targeting in comparison to a random integration. Clones obtained by non-targeted integration correspond to clones that show a fluorescence signal similar to the signal obtained for CHO-M wild-type cells: form this it was inferred that the integration of the transgene had occurred at random genomic sequences as it was not targeted to the ERV locus.

It is noted in this example that, upon HR stimulation and NHEJ inhibition, transgene integration with DNA homology resulted in a higher transgene integration frequency in the ERV-containing allele than without DNA homology. This may reflect the fact that homologies represent a help for targeted transgene integration by homologous recombination. However, the highest frequency of targeted integration at both alleles occurred when using expression vectors without homology, and upon Alt-EJ stimulation and NHEJ inhibition (FIG. 6).

In conclusion, a high efficiency process for transgene integration with CRISPR and gRNA has been designed here: between 70% and 90% higher targeted integration is observed relative to a non-targeted integration. Furthermore, stimulation of either the HR or Alt-EJ pathway did, in this example, increase targeted integration efficacy, depending upon the presence or not of DNA homology, for integration in the ERV-containing or in both alleles.

FIG. 7A and FIG. 7B show chromosomal distribution frequency histograms of the DNA-FISH signal in pool 1 and the pool 3 (for pool composition see Table 4) respectively: Cells from the pool 1 and the pool 3 populations were blocked in metaphase using colcemid and were spread on glass slides. Then, a DNA-FISH experiment was performed on each sample using a probe targeting the promoter that drives the expression of the GFP (green fluorescent protein); Images were collected using a confocal microscope Zeiss LSM800. Finally, the images were analyzed using Karyotype-analyzer and karyotypes were generated. Targeted integration in chromosome 15 (Chr15) and chromosome 9 (Chr9) were enriched up to 60% and 19.5% in the pool 1, and up to 45% and 32% in the pool 3. The n values indicate the number of karyotypes analyzed.

In both pools, several transgene integration sites were observed on other chromosomes, but at a much lower frequency, since the total random transgene integration events represent about 20%.

Finally, this result confirmed that CRISPR cleavage combined to the inhibition of NHEJ and to the activation of the HR or especially the Alt-EJ MMEJ mechanisms, allow highly efficient targeted transgene integration in chromosome 15 and chromosome 9.

These results reveal that targeted integration can be highly efficient upon Alt-EJ activation, with up to about 80% altogether of integrations happening in the Chromosome 9 and/or 15 portions that contain the two alleles of the ERV-109F locus, i.e., the ERV-devoid WT and ERV C 109F-containing allele. CHO-M cells do not have homologous sequences for all chromosomal loci on homologous chromosomes because, as for all CHO cells, they were subjected to extensive chromosomal rearrangement during their selection for optimal growth properties in vitro following their isolation from native Chinese Hamster cells. This explains why homologous sequences of the genome may be located on distinct chromosomes, as observed for the examined locus.

FIG. 7C shows representative karyotypes for transgenic insertion happening in chromosome 9 and chromosome 15 respectively for cells from Pool 1, with the DNA stained in grey and DNA-FISH probes directed against telomeric repeats (Sequence TTAGGGTTAGGGTTAGGG [SEQ ID NO: 60]) and against the promoter driving the expression of the GFP stained in white. White arrows indicate the position of the FISH signal for the ERV and the WT alleles.

### Example 2: Increase of Transgene Expression by Targeting the ERV C 109F Locus

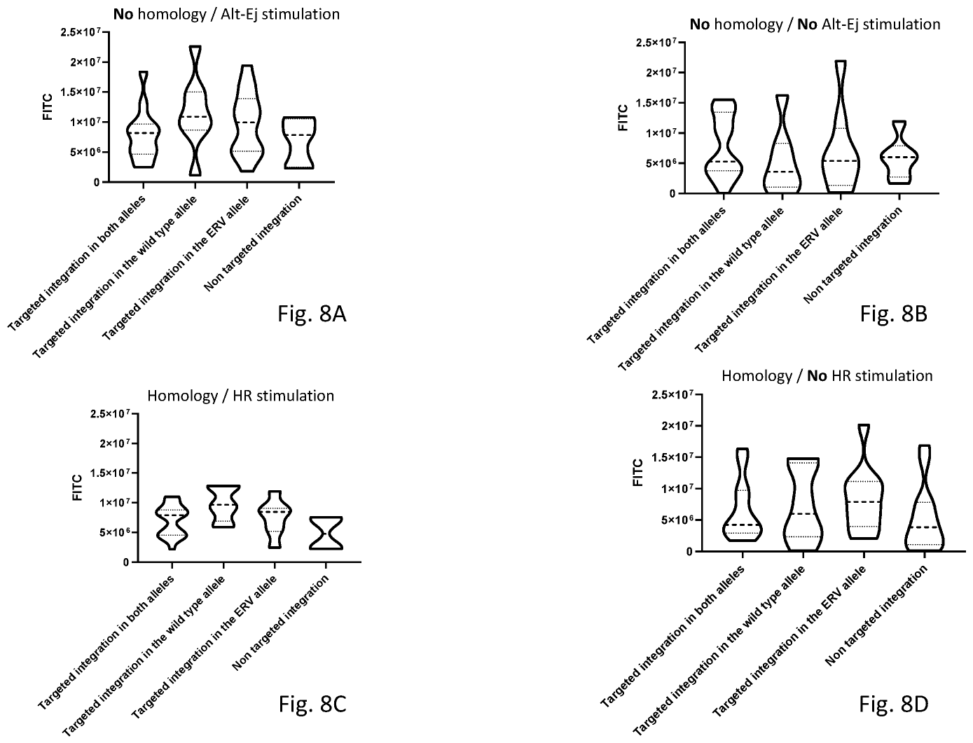

This example illustrates that the ERV C 109F locus allows enhanced and stable expression of exogenous transgenes. FIG. 8 shows an analysis of GFP-expressing CHO clones, as based on FITC fluorescence analysis. 340′000 cells were transfected per condition and plated 2 days after transfections on semi-solid medium in presence of antibiotic selection. 42 clones were picked (ClonePix®) based on fluorescence intensity and cultivated. 9 days after picking, FITC was measured (Cytoflex®) on 2000 cells per clones, and the types of transgene integration events were determined for each clone by qPCR analysis (TaqMan®). The 42 clones were put into 4 categories, with the transgene being located either in the wild allele, in the ERV C 109F-containing allele, in both alleles, or upon non-targeted integrations corresponding to off-target genomic integration events, as before.

FIGS. 8A, B, C and D show the fluorescence intensity (FITC) depending on the transgene locus integration type. The representation of the fluorescence for each type of integration allowed to check if one locus provides higher transgene expression compared to the others and if the modulation of repair pathways alters transgene expression levels.

The results globally show that targeting the ERV C 109F locus appears to mediate higher FITC fluorescence when compared to random genomic integration represented by the non-targeted integrations. It can be even seen that integration into the wild type allele of the ERV C 109F locus yielded higher fluorescence levels than the ERV allele upon DNA repair pathways modulation (e.g. FITC fluorescence of the clones isolated from pools 1 and 3, FIGS. 8A and 8C). The integration in the wild type allele of the ERV C 109F locus yielded also higher fluorescence when compared to integration in both alleles. Thus, in certain embodiments of the present invention, an integration into the wild type allele of the ERV C 109F locus without integration into the corresponding ERV occupied allele is preferred. This embodiment is in particular preferred if a potential negative impact may occur following integration in the ERV-containing allele, and/or if the WT ERV-devoid allele is more favorable for high-level and stable transgene expression than the ERV-containing allele. As the person skilled in the art will appreciate, also in an embodiment in which there is no transgene integration in the ERV-containing allele, the ERV sequence is in certain embodiments modified to eliminate or reduce the release of viral particles from the cell, preferably so that a release of the viral particle is not detectable anymore (see Duroy, 2020).

Modulation of DNA repair pathways is also particularly preferred in certain embodiments, as it appears also to provide an enhanced transgene expression at this locus, in particular when applying the Alt-EJ modulation that gives a clearly higher expression than without this modulation (e.g. see the dotted lines indicating median fluorescence values in FIGS. 8A and 8B).

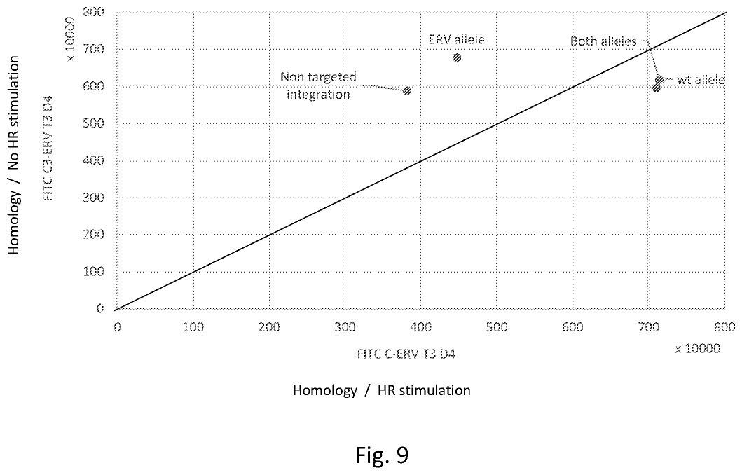

FIG. 9 shows results obtained with another transfection and another batch of clones obtained from pool 3 and pool 4. These results show that the fluorescence obtained from the clones generated using targeted integration with DNA homology, when comparing cells generated using HR modulation (pool 3, FIG. 8C) to the absence of HR modulation (pool 4, FIG. 8D).

These results validate the previously obtained results by showing that the modulation of the HR repair pathway can also be used to generate cell clones displaying an increased transgene expression, especially when the transgene is integrated in the WT allele or into both alleles when compared to integration at the ERV-containing allele solely or to non-targeted integration. This result further validates that the modulation of DNA repair mechanisms is advantageous for obtaining increased transgene expression.

### Example 3: Expression of Complex Proteins

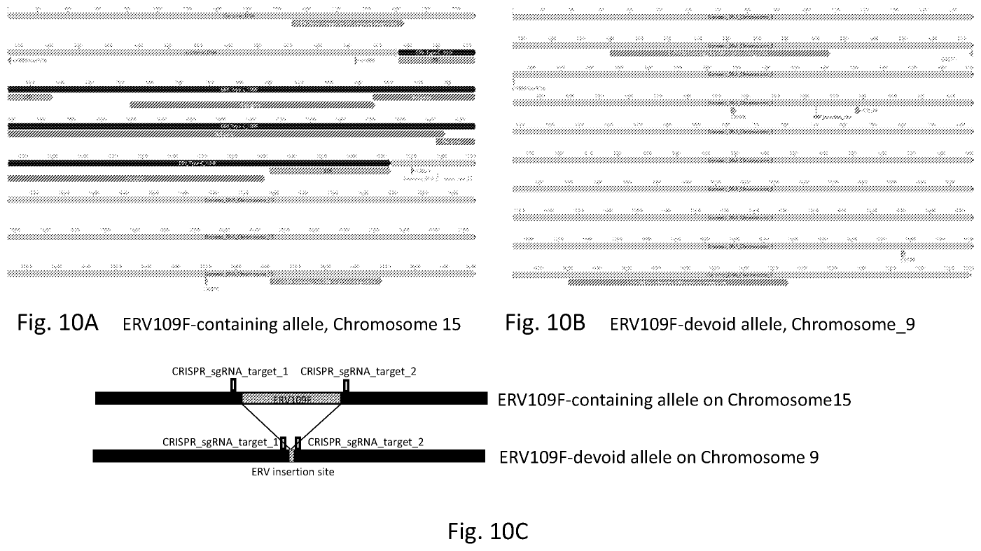

This example shows that the set-up that allowed GFP expression upon integration into the chromosome 9 ERV109F-devoid WT allele and/or at the homologous chromosome 15 ERV109F-containing allele (FIG. 10), also allowed for the expression of the Trastuzumab therapeutic protein.

Initially, CHO cells were transfected with the expression vectors for CRISPR-mediated cleavage together with Trastuzumab expression vectors (FIG. 11B). For some of the transfections, vectors mediating the expression of the POLQ, MRE11 and CIRBP proteins that increase alternative end-joining mechanisms such as the microhomology-mediated end-joining (MMEJ) pathway were also included (FIG. 11B). This was done with the goal of promoting the integration of the Trastuzumab expression vectors at the chromosomal CRISPR cleavage sites, as the Trastuzumab expression vectors do not show significant DNA sequence homology with chromosomal loci, since they do not bear homology sequences. Cells containing genome-integrated transgene sequences were selected for puromycin antibiotic resistance, whereafter clonal populations were isolated using the ClonePix™ FL Imager from Molecular Devices, LLC.

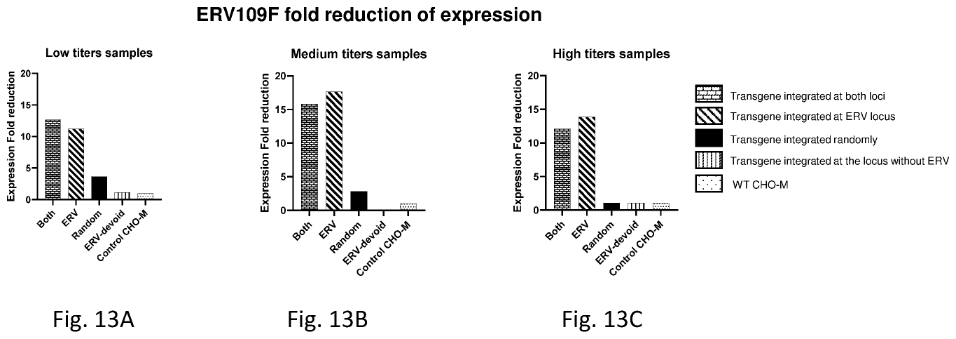

The derived clones were then analyzed by PCR assays to determine at which genomic loci the transgenes were integrated, using the primers illustrated by arrows in FIG. 12: Amplification with the primers indicated by arrows in FIG. 12B show that no transgene insertion occurred in the ERV-devoid WT allele (in the Figs. referred to as locus without ERV). The primers indicated in FIG. 12C assess whether the ERV is still inserted at its regular locus with no transgene inserted. Amplification with all primers indicated in FIG. 12D shows that both alleles are intact and that transgene integration must have occurred elsewhere in the genome. In FIG. 12A, the lack of amplification with either primer pair shows insertion in both alleles. Thus, four types of clones were identified, containing the transgene integrated either at random positions, at the ERV109F-containing chromosome 15 allele, at the ERV109F-devoid chromosome 9 WT allele, or at both chromosome 15 and 9 alleles. As expected, clones having integrated the Tras transgene at the ERV-containing allele or at both alleles, thus replacing the ERV109F sequence by the Tras-coding sequence, had the strongest reduction in the ERV expression, up to over 17-fold reduction, so as to reach undetectable levels within the PCR background (FIGS. 13A-C).

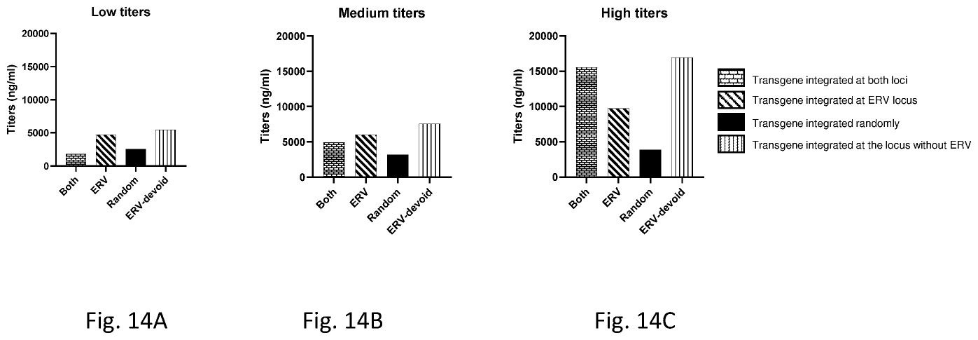

Clones were then screened for Trastuzumab secretion, and representative clones expressing Trastuzumab at low (FIG. 14A), moderate (FIG. 14B), and high levels (FIG. 14C), were then selected for each clone type. As observed for GFP expression, the highest levels of Trastuzumab production levels were obtained upon the integration of the Tras coding sequence into the ERV-devoid chromosome 9 locus, followed by clones having the Tras sequence integrated into both loci. The production levels obtained from targeted integration at the ERV109F-containing and/or -devoid WT alleles were much higher than those obtained from random genomic integration. Overall, these findings indicated that the ERV locus genomic environment is highly favorable for gene expression, and that the most productive clones are obtained upon transgene integration at the ERV-devoid Chr 9 WT allele.

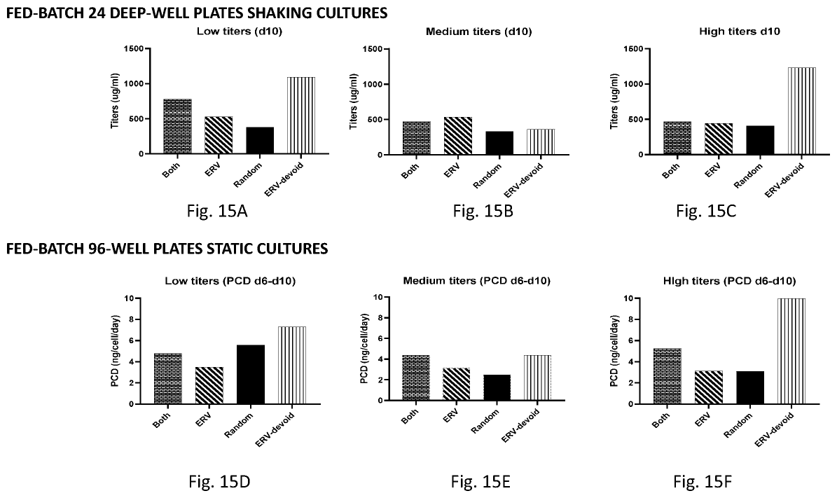

Next, it was assessed whether the high Tras titers obtained in the supernatants of small scale and short duration non-fed cultures may translate into therapeutic protein production-like cultures. The specific productivity levels obtained during the 6 to 10 days interval of small scale 96-well plates (FIGS. 15D-F), as well as the titers obtained from the supernatants of fed-batch cultures performed in shaken 24-well plates (FIGS. 15A-C), indicated that the optimal Tras expression was obtained from transgene integration into the ERV-devoid WT allele.

Upscaling

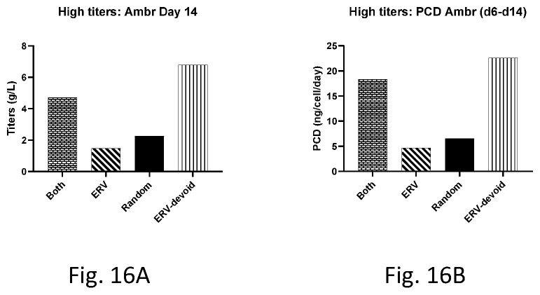

The productivity of the clones mediating the highest titers for each type of genomic integration (FIGS. 15C and 15F) were assessed at a larger scale, using fed-batch cultures performed in Ambr® 15 bioreactors. Specific productivities above 20 picogram of secreted Tras IgG per cell and per day were obtained for the clone having integrated the transgene at the ERV-devoid wt allele (FIG. 16B). This clone mediated also the highest titer of antibody released into the cell culture supernatant (FIG. 16A).

Overall, it was surprisingly found that the optimal targeted integration locus for high transgene expression, and for optimal production of therapeutic proteins, are the chromosomal loci where a highly expressed ERV integrated, more preferably the ERV109F-containing chromosome 15 genomic allele, and, more even more preferably, the ERV109F-devoid WT allele on chromosome 9. Expression vectors for alternative end-joining factors like the MRE11 and PoIQ MMEJ proteins (see FIG. 11A) can be and were co-transfected with the therapeutic protein vectors, so as favor higher frequencies of targeted integration of the non-homologous plasmid sequences at this favorable genomic locus. Double integration of the therapeutic protein expression vectors at both the ERV-containing and ERV-devoid WT alleles is also highly favorable, allowing, as a result of a single transfection, high level production of the therapeutic protein as well as abolished expression of potentially harmful retroviral sequences that lead to the release of viral particles into the cell supernatant, together with the therapeutic protein of interest.

Material and Methods

Cell Culture

Suspension-adapted Chinese hamster ovary (CHO-M) derived cells were maintained in serum-free BalanCD CHO medium (Irvine Scientific) supplemented with L-glutamine (GE Healthcare). CHO-M viable cell density and fluorescence signal of green fluorescent transfected cells were assessed using the Cytoflex Flow Cytometer (Beckman Coulter). Cells were cultivated in 50 ml C50 bioreactor tubes (TPP, Switzerland) at 37° C., 5% C02 in a humidified incubator with 180 rpm agitation speed and passaged every 3-4 days.

Plasmids Construction

Two EGFP (Enhanced Green Fluorescent Protein) expression vectors were used in this study. The two vectors have the same eukaryotic expression cassette composed of an antibiotic resistance cassette followed by the EGFP expression cassette with a downstream SV40 enhancer and a SELEXIS Genetic Element (SGE). SELEXIS SGE are unique epigenetic DNA-based elements that control the dynamic organization of chromatin across all mammalian cells. They allow for enhanced transcription by isolating the integrated transgene from the silencing effects of the surrounding chromatin.

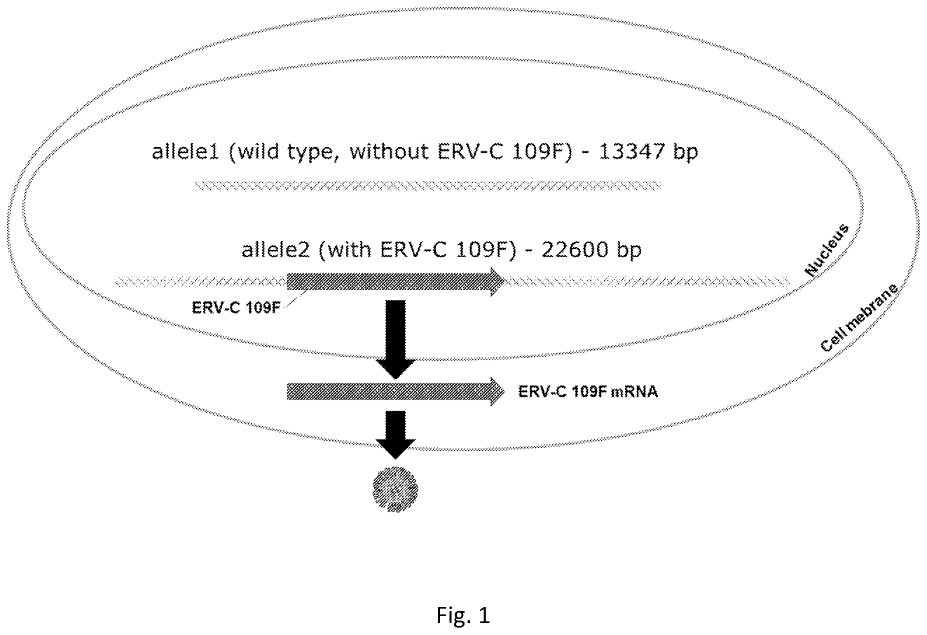

Duroy et al. 2019 described that only one Type-C ERV among the 173 identified in the CHO genome is able to be transcribed and able to produce viral particles present in the CHO culture supernatant. The locus of integration is specific because this ERV sequence is only present at an hemizygous state in the CHO cell genome (FIG. 1). It means that one allele is present in the CHO genome without ERV integration (called the wild type (WT) or ERV-devoid WT allele), and another allele at an homologous DNA sequence within the CHO genome where the integrated ERV can be found (called the ERV-C 109F-containing allele, or ERV-C 109F allele). Only the last allele will express the corresponding viral sequences in the cell, resulting in the presence of ERV-C 109F mRNA in the cytoplasm.

One of the EGP bearing vectors used in this study contains, in addition, two homology sequences of 750-bp long that correspond to two DNA sequences from the genomic locus around the ERV C 109F-containing and ERV-devoid WT alleles (SEQ ID 6 and SEQ ID 7), which are positioned on each side of the allele breakpoint and CRISPR cleavage sites (5′ and 3′ homology arms) as described in FIG. 2.

Two sets of CRISPR vectors were used in addition to the EGFP vectors to introduce site-specific DSBs. The CRISP 16 and CRISP 17 DSB (SEQ ID 8 and SEQ ID 9) are preferably repaired by the homologous recombination pathway using the 5′ and 3′ homology arms, which are present in the vector and in the WT allele of the locus and/or ERV-containing allele as homology sequences. The CRISP 50 and CRISP 51 DSB (SEQ ID 10 and SEQ ID 11) are preferably repaired by the Alt-EJ pathway using micro-homology sequences present in the vector and in the wild type allele.

Transfection and Single Cells Isolation

For the inhibition of the NHEJ DNA repair pathway, CHO-M cells growing in suspension were pre-treated with 0.5 μM Nu7441 to inhibit DNA-PKcs. Cells for which the homologous recombination (HR) pathway was stimulated were in addition treated with 1 μM RS-1. Pre-treated cells were transfected (340′000 cells/transfection) with the two expression vectors containing the Enhanced Green Fluorescent Protein (EGFP) coding sequence for ease of detection, as presented in FIGS. 3 and 4. In order to stimulate an Alt-EJ repair pathway, namely the MMEJ pathway, cells were also transfected with hMRE11 (SEQ ID 25), cgPOLQ (SEQ ID 26) and hCIRBP (SEQ ID 27) genes cloned into separate expression vectors. Whereas in order to stimulate the HR repair pathway, cells were also transfected with hMRE11 and hRAD51 (SEQ ID 28) genes cloned into separate expression vectors.

One day after transfections, cells were centrifuged and medium was exchanged in order to remove Nu7441 and RS-1. Two days after transfections, cells were plated at a cell density of 5000 cells/ml on semi-solid medium containing 3 μg/ml of puromycin. After 10 days of growth in semi-solid medium, 42 EGFP expressing clones per transfection were picked (ClonePix, Molecular Devices) based on fluorescence intensity and cultivated in BalanCD CHO medium. Nine days after picking (experience with DNA repair pathway stimulation) and 6 days after picking (experience without DNA repair pathway stimulation), EGFP expression level (FITC) was measured (Cytoflex®) on 2000 cells per clone. Results were displayed by categories determined by qPCR analysis (TaqMan®).

TagMan® qPCR Assays

DNA Extraction

Genomic DNA (gDNA) was extracted from 2×10E6 cells using the CellsDirect One-Step qRT-PCR Kit® (ThermoFisher Scientific®) following the manufacturer instructions. gDNA quantification was conducted using the NanoDrop® spectrophotometer (ThermoFisher Scientific®).

qPCR Assays

Three Taqman® qPCR assays were designed (FIG. 5A and FIG. 5B; probes and primers are depicted form SEQ ID NO:29 to SEQ ID NO: 37). Linearity and efficiency of all TaqMan qPCR assays were verified using the standard curve approach. All assays were highly linear (=0.999) and showed very good efficiency (≥0.97).

qPCR runs were performed on QIAGEN's Rotor-Gene using the Rotor-Gene Multiplex PCR Kit® and FAM or HEX-labeled TaqMan® qPCR assays. Data analysis was performed using the Rotor-Gene Q Series® Software (v2.3.1).

Specificity of TaqMan® qPCR assays for the three loci validation were performed using appropriate negative controls and using the standard curve approach in order to validate absence or presence of the reference locus.

Absence or presence of one amplicon at a CT range corresponding to the control allowed to determine if the target locus of integration of the ERV containing allele or in the Wild Type allele of the locus of the ERV integration were like the non-transfected CHO-M cells. And the results “yes or no” for the three different TaqMan assays allow to determine in which category is each clone.

Fluorescent In-Situ Hybridization Experiments

Cells were blocked in metaphase using colcemid and were spread on glass slides. DNA-FISH experiments were performed on each sample using a probe targeting the promoter that drives the expression of the EGFP (green fluorescent protein) vector. Images were collected using a confocal microscope Zeiss LSM800. Finally, the images were analyzed using a Karyotype-analyzer and karyotypes were generated.

Generation and Characterization of Trastuzumab Producing Cell Clones

CHO-M cells were co-transfected with the PuroBT+_Tras_Hc and the PuroBT+_Tras_Lc Trastuzumab (Tras) immunoglobulin (IgG) expressions plasmids (FIG. 11B) and the CRISPR-sgRNA vectors to allow for the targeted integration of the IgG-encoding vector at the ERV109F integration genomic locus. Cells were selected for resistance to puromycin and single cell-derived clones were isolated using the ClonePix™ FL Imager from MOLECULAR DEVICES, LLC.

CHO-M Cell Line and Fed-Batch Cultivation

Parental Selexis CHO-M cells and derived clonal cell lines stably expressing the human monoclonal IgG1 antibody were cultured as follows: Seed train cultures were passaged every 3 to 4 days prior to N−1 seed. Four days before microbioreactor inoculation, CHO-M cultures were passaged in shake flasks at a seeding cell density of 0.30×10⁶ cells/ml (N−1) at a volume according to process needs. Cells were cultivated in the chemically defined BalanCD Growth A® culture medium (IRVINE SCIENTIFIC, USA) supplemented with 6 mM L-Glutamine (HyClone, USA) with an incubator (KÜHNER, Germany) settings at 37.0° C., 5% CO₂ and 120 rpm.

Total Protein Quantification Assays

An automated microfluidic capillary gel electrophoresis system, the LabChip LCGXII system (PERKIN ELMER, Inc.), was used for the total protein assays. Protein-containing samples were mixed with an amine-reactive fluorescent dye that labels proteins non-specifically and proteins were detected with laser-induced fluorescence at the outlet of the separation channel.

Clone Characterization for Targeted Integration Efficacy

The genomic integration site of the Tras expression vectors of cell clones producing the IgG were analyzed by q-PCR assays performed on genomic DNA. quantitative-PCR (q-PCR) were carried in multiplex using 3 Taqman probes. Two Taqman assays were designed to determine the presence of the ERV109F junction sequences between ERV and genomic DNA on each side of the ERV integration locus on chromosome (Chr) 15. A lack of amplification indicated that one or several transgene copies had integrated at the ERV109F allele and that the ERV sequence had been deleted (FIGS. 12A and 12B). The third Taqman assay was designed to assess the presence of the WT allele, i.e. the ERV-devoid Chr 9 genomic sequence, to assess transgene integration at this locus (FIGS. 12A and 12C).

If no product was obtained from the three q-PCR assays, it could be deduced that transgene copies had integrated at both alleles (FIG. 12A). If all three Taqman assays yielded positive results, this indicated that both alleles were intact and that the Tras-encoding sequences had therefore integrated elsewhere in the genome (FIG. 12D). Thus, such clones were classified as ones in which transgene integration had occurred randomly.

ERV109F Expression

Total cellular RNA was extracted using the RNeasy® kit by QIAGEN following the manufacturer's protocol. Two DNAse treatment were performed during and after the extraction. The GoScript® reverse transcriptase (RT) kit by PROMEGA was used to reverse-transcribe the RNAs into DNAs.

RT-qPCR assays of the ERV109F RNA of the Tras-producing clones and parental CHO-M cells were performed using a Taqman assay designed to detect the ERV109F Long Terminal Repeat sequence, using the cellular GAPDH housekeeping gene mRNA as a reference. Determination of the fold decrease of ERV109F expression was made following the delta delta CT calculation method (Livak and Schmittgen, Analysis of Relative Gene Expression Data Using Real-Time Quantitative PCR and the 2-ΔΔCT Method, Methods 25, 402-408, 2001). This assay allowed the determination of the ERV expression decrease following the transgene integration at the ERV locus, thus further validating transgene integration at the ERV109F locus and ERV sequence deletion.

Cultures and Assays of the Trastuzumab Antibody Production Ability of Cells Clones

The cells cultivation process used to determine Tras production in fed-batch cultures were performed as follows: Cell growth and production performance were evaluated using classical fed-batch static cultures in 96 deep well plate, or in 24 deep well plate under stirring. Fed-batch cultures in an Ambr15® automated microscale bioreactor system (SARTORIUS Stedim, Germany) equipped with a cooling system to allow temperature shift was also performed. All cultures were carried out with 40% of dissolved oxygen (DO), stirring speed between 1000 to 1400 rpm, temperature maintained at 36.5° C. then shifted at 33.0° C. (time shift according to seeding density) and pH controlled at 6.90±0.10 then shifted at 7.00±0.20 using CO2 and 1M carbonate (time shift according to seeding density).

Fed-batch culture in 24 deep wells or 96 deep wells were seeded at a target cell density of 300 000 cells/ml, using culture volumes of 3 ml and 250 ml respectively. Microbioreactors were seeded at a target cell density of 1.00×10⁶ cells/mL in 13 mL initial working volume depending on the seeding density process in Ambr15. Cell culture supplement1 and Cell culture supplement 2 feed supplements were added to cultures at various days, depending on the seeding density process. Glucose solution (SIGMA ALDRICH, USA) was added as based on the daily glucose concentration. As needed to maintain a good cell viability and high-level production. Microbioreactor samples were harvested daily for cell counting, and viable cell density (VCD) determination. Cell viability was measured using a Bioprofile® FLEX2 (NOVA BIOMEDICAL, USA). Cells were grown for up to 14 days.

As the person skilled in the art will appreciate, the above description is not limiting, but provides examples of certain embodiments of the present invention. With the guidance provided above, the person skilled in the art is able to devise a wide variety of alternatives not specifically set forth herein.

### BIBLIOGRAPHY

Duroy et al., Characterization and mutagenesis of Chinese hamster ovary cells endogenous retroviruses to inactivate viral particle release. Biotechnol Bioeng. (published online October 2019)
US Patent Publication 2020-0109421A1
Havecker et al., The diversity of LTR retrotransposons, Genome Biology 2004, 5:225 (2004)
Bell and Felsenfeld, Stopped at the border: boundaries and insulators. Curr Opin Genet Dev 9, 191-198 (1999).
Zahn-Zabal et al., Development of stable cell lines for production or regulated expression using matrix attachment regions, J. Biotechnol. 87: 29-42 (2001)
Kim et al., Regulation of Swi6/HP1-dependent heterochromatin assembly by cooperation of components of the mitogen-activated protein kinase pathway and a histone deacetylase Clr6. J. Biol. Chem.; 279: 42850-42859 (2004)
Agarwal et al., Scaffold attachment region-mediated enhancement of retroviral vector expression in primary T cells. J Virol 72, 3720-3728 ((1998)
Castilla et al., Engineering passive immunity in transgenic mice secreting virus-neutralizing antibodies in milk, Nature Biotech. Vol. 16, 349-354 (1998)
Taher and Ovcharenko, Variable locus length in the human genome leads to ascertainment bias in functional inference for non-coding elements, Bioinformatics, Vol. 25 no. 5 2009, pages 578-584 (2009)
Leahy et al., Identification of a highly potent and selective DNA-dependent protein kinase (DNA-PK) inhibitor (NU7441) by screening of chromenone libraries. (Bioorg. Med. Chem. Lett. 14:6083-6087 (2004)
Willmore et al., A novel DNA-dependent protein kinase inhibitor, NU7026, potentiates the cytotoxicity of topoisomerase II poisons used in the treatment of leukemia. Blood 103 (12):4659-65 (2004)
Maruyama et al., Increasing the efficiency of precise genome editing with CRISPR-Cas9 by inhibition of nonhomologous end joining, Nat. Biotechnol. 33:538-542 (2015)
Robert et al., Pharmacological inhibition of DNA-PK stimulates Cas9-mediated genome editing, Genome Med 7:93 (2015)
Dittmann et al., Inhibition of radiation-induced EGFR nuclear import by C225 (Cetuximab) suppresses DNA-PK activit, Radiother and Oncol 76: 157 (2005)
Shibata et al, inhibitors of PoIQ, inhibitors of Ctl, Molec. Cell 53:7-18 (2014)
Sfeir and Symington, “Microhomology-Mediated End Joining: A Back-up Survival Mechanism or Dedicated Pathway?” Trends Biochem Sci 40:701-714 (2015)
Urnov F., et al., Highly efficient endogenous human gene correction using designed zinc-finger nucleases Nature 435:646-651 (2005)
Mussolino et al., A novel TALE nuclease scaffold enables high genome editing activity in combination with low toxicity Nucl. Acids Res. 39(21):9283-9293 (2011)
Gao et al., DNA-guided genome editing using the Natronobacterium gregoryi Argonaute, Nature Biotechnology, (published online May 2, 2016)
Swarts et al., DNA-guided DNA interference by a prokaryotic Argonaute, Nature 507(7491): 258-261 (2014)
Sheng et al, Structure-based cleavage mechanism of Thermus thermophilus Argonaute DNA guide strand-mediated DNA target cleavage, Proc. Natl. Acad. Sci. U.S.A. III, 652 (2013).
Boissel et al., MegaTALs: a rare-cleaving nuclease architecture for therapeutic genome engineering, Nucleic Acids Research 42 (4):2591-2601 (2013)
Kay et al., A bacterial effector acts as a plant transcription factor and induces a cell size regulator, Science 318:648-651 (2007)
Computational Molecular Biology, Lesk, A. M., ed., Oxford University Press, New York (1988)
Biocomputing: Informatics and Genome Projects, Smith, D. W., ed., Academic Press, New York (1993)
Computer Analysis of Sequence Data, Part I, Griffin, A. M., and Griffin, H. G., eds., Humana Press, New Jersey (1994)
Sequence Analysis in Molecular Biology, von Heinje, G., Academic Press (1987)
Sequence Analysis Primer, Gribskov, M. and Devereux, J., eds., M Stockton Press, New York (1991)
Carillo, H. & Lipton, D., The Multiple Sequence Alignment Problem in Biology, SIAM J Applied Math 48:1073 (1988)
Smith and Waterman, Advances in Applied Mathematics 2:482-489 (1981)
Brutlag et al., Comp. App. Biosci. 6:237-245 (1990)
Altschul et al., J. Mol. Biol. 215:403-410 (1990)
H. Neurath and R. L. Hill, In: The Proteins, Academic Press, New York (1979)

1. An engineered cell comprising:
within the genome of the cell:
at least one locus comprising an insertion site of an endogenous retrovirus (ERV) sequence or a LTR-retrotransposon (LTR-RT) sequence, wherein said at least one locus comprise(s):
(i) a) the ERV sequence or the LTR-RT sequence, or b) the insertion site and, optionally, parts of the sequence of a), and/or
(ii) an allelic wild type counterpart sequence of (i), and
at least one transgene encoding at least one transgene expression product integrated into the at least one locus.

2. The engineered cell of claim 1, wherein the cell comprises (i) and (ii) on different chromosomes including chromosome 15 and 9 of a CHO cell.

3. The engineered cell of claim 1, wherein the cell comprises (i) and (ii), and the at least one transgene is integrated into (ii).

4. The engineered cell of claim 3, wherein the at least one transgene is not integrated into (i).

5. The engineered cell of claim 3, wherein the at least one transgene is also integrated into (i).

6. A cell population comprising engineered cells according to claim 1, wherein the at least one transgene is integrated into more than 20%, 30% or even more than 40% of (i) and/or (ii) of cells within said cell population.

7. The cell population of claim 6, wherein the cell comprises (i) and (ii), wherein (i) comprises: at least nucleotides 29021 to 40247 of SEQ ID NO: 1 or a sequence having 95%, 98% or 99% sequence identity with nucleotides 29021 to 40247 of SEQ ID NO: 1 and (ii) comprises at least nucleotides 29020 to 31020 of SEQ ID NO: 2 or a sequence having 95% 98%, or 99% sequence identity with nucleotides 29020 to 31020 of SEQ ID NO: 2, or
at least nucleotides 29521 to 39747 of SEQ ID NO: 1 or a sequence having 95%, 98% or 99% sequence identity with nucleotides 29521 to 4247 of SEQ ID NO: 1 and (ii) comprises at least nucleotides 29520 to 30520 of SEQ ID NO: 2 or a sequence having 95% 98%, or 99% sequence identity with nucleotides 29520 to 30520 of SEQ ID NO: 2.

8. The cell population of claim 6, wherein one or more cells of the cell population or the cell population lack(s) expressed endogenous retrovirus (ERV) sequences and/or there are no detectable viral particles comprised in a culture supernatant of the cell population.

9. The cell of claim 1, wherein the at least one transgene expression product is a protein of interest and the cell or a cell population comprising one or more of the cells optionally expresses the protein of interest per unit of time, in an amount that exceeds the amount of a protein of interest when the at least one transgene is integrated into the genome outside the at least one locus, by at least 1.5 fold, 2 fold, 2.5 fold, 3 fold or more.

10. The engineered cell of claim 1, wherein the ERV or LTR-RT is selected from the group consisting of a type C endoretroviral element (ERV C), MLV (murine leukemia virus), XMRV (xenotropic murine leukemia virus-related virus), MMTV (mouse mammary tumor virus), MERV-L (mouse ERV with L-tRNA PBS), VL30 (virus like 30), IAP (intracisternal A-type particle), MusD (Mus type-D related retrovirus), PERVs (porcine endogenous retroviruses), KoRV (koala retrovirus), enJSRV (Jaagsiekte sheep retrovirus), MaLR (mammalian apparent LTR retrotransposons), HERV (human endogenous retroviruses) such as HERV-E (human ERV with E-tRNA PBS), HERV-H (human ERV with H-tRNA PBS), HERV-K (human ERV with K-tRNA PBS), HERV-L (human ERV with L-tRNA PBS), HERV-W (human ERV with W-tRNA PBS) and combinations thereof.

11. The engineered cell of claim 1, wherein the ERV or LTR-RT sequence comprises at least one ERV subsequence selected from the group consisting of a gag (group-specific antigen) gene, a pol (polymerase) gene, an env (envelope) gene, a sequence encoding a MA (matrix), a CA (capsid), a NC (nucleocapsid), a sequence encoding a SP1 (Spacer peptide 1), a sequence encoding a SP2 (Spacer peptide 2) or a further domain encoding proteins including pp12 or p6, are long terminal repeats (LTRs) of a ERV and combinations thereof and wherein the transgene is optionally integrated into one of the subsequences.

12. The engineered cell of claim 1, wherein the cell is transfected with one or more vectors comprising one or more genes of Table 2 or SEQ ID Nos: 25-28, 38-58 and/or 59 or sequences having at least 80%, 85%, 90%, 95%, 98% or 99% sequence identity with SEQ ID Nos: 25-28, 38-58 and/or 59, wherein a cytoplasm of the cell optionally further comprises exogenous chemical inhibitors and/or stimulators of one or more DNA Repair Pathways (DRPs), including NHEJ inhibitors selected from the group consisting of NU7441, Olaparib, DNA Ligase IV inhibitor, Scr7 KU-0060648 anti-EGFR-antibody C225 (Cetuximab), Compound 401 (2-(4-Morpholinyl)-4H-pyrimido[2,I-a]isoquinolin-4-one), Vanillin, Wortmannin, DMNB, IC87361, LY294002, OK-1035, CO 15, NK314, PI 103 hydrochloride and combinations thereof, MMEJ inhibitors selected from the group of Mirin, derivatives of Mirin, inhibitors of PoIQ, inhibitors of CtIP and combinations thereof, HR inhibitors including RI-1 and BO2, HR stimulators including RS-1, NHEJ stimulators, including IP6 and combinations of any one of the above inhibitors and/or stimulators.

13. The engineered cell of claim 1, wherein the locus has at least 80%, 90%, 95%, 96%, 97%, 98% or 99% sequence identity with a sequence selected from SEQ ID Nos. 1 and/or SEQ ID No. 2.

14. The engineered cell of claim 1, wherein the transgene is a landing pad.

15. The engineered cell of claim 1, wherein the cell is a Chinese Hamster Ovary (CHO) cell, a human cell or a porcine cell.

16. A method for transgene integration into a genome of a cell, preferably of a mammalian cell line comprising:
(a) providing at least one transgene as part of a vector, such as a plasmid or viral vector, comprising the at least one transgene, wherein the vector integrates the transgene into a least one locus of the cell comprising an insertion site of an endogenous retrovirus (ERV) sequence or a LTR-retrotransposon (LTR-RT) sequence, or
(b) providing at least one transgene, optionally as part of a vector, and at least one nuclease and/or nickase, wherein the nuclease and/or nickase is preferably encoded by at least one vector, wherein the nuclease and/or nickase introduces, for integration of said transgene therein, double and/or single strand breaks into a least one locus of the cell comprising an insertion site of an endogenous retrovirus (ERV) sequence or a LTR-retrotransposon (LTR-RT) sequence and optionally, providing at least one vector encoding at least one targeting element guiding said least one nuclease and/or nickase, optionally, upmodulating, in particular stimulating at least one first DNA Repair Pathway (DRP) of the cell and optionally downmodulating, in particular stimulating at least one second DRP of the cell, or vice versa,
transfecting the cell with the at least one transgene; and
optionally isolating an engineered cell that comprises the at least transgene integrated into the locus.

17. The method of claim 16, wherein the cell is also:
transfected, preferably as part of one or more further vector(s), with one or more genes of Table 2 or SEQ ID Nos: 25-28, 38-58 and/or 59 or sequences having at least 80%, 85%, 90%, 95%, 98% or 99% sequence identity with SEQ ID Nos: 25-28, 38-58 and/or 59 and/or
brought in contact with a chemical affecting the DNA Repair Pathway (DRP) of the cell.

18. The method of claim 16, wherein the cell is transfected with one or more further vector(s) comprising and expressing SEQ ID Nos: 25-28, 38-58 and/or 59, preferably SEQ ID Nos: 25-28.

19. The method of claim 16, wherein the at least one nuclease and/or nickase is:
a transposase, an integrase, a recombinase such as a site-specific recombinase, a nickase, or a nuclease such as a site-specific nuclease, a fusion protein comprising a programmable DNA-binding domain and a nuclease domain or any combinations thereof, or
a homing endonuclease, a restriction enzyme, a zinc-finger nuclease or a zinc-finger nickase, a meganuclease or a meganickase, a transcription activator-like effector nuclease or a transcription activator-like effector nickase, an RNA-guided nuclease or an RNA-guided nickase, a DNA-guided nuclease or a DNA-guided nickase, a megaTAL nuclease, a BurrH-nuclease, an ARCUS nuclease, a modified or chimeric version or variant thereof, and combinations thereof, in particular a zinc-finger nuclease or a zinc-finger nickase, a transcription activator-like effector nuclease or a transcription activator-like effector nickase, a RNA-guided nuclease or a RNA-guided nickase, wherein the RNA-guided nuclease or an RNA-guided nickase is optionally part of a CRISPR-based system, restriction enzyme and combinations thereof.

20. The method of claim 19, wherein the recombinase is a Cre recombinase, FLP recombinase, lambda integrase, PhiC31 integrase, Dre recombinase, xb1 integrase, gamma delta resolvase, R4 integrase, Tn3 resolvase, or TP901-1 recombinase.

21. The method of claim 19, wherein said nuclease is a transcription activator-like effector nuclease or a RNA-guided nuclease.

22. The method of claim 16, wherein the viral vector is an AAV vector.

23. The method of claim 16, wherein the first and/or second DRP is selected from the group consisting of resection, canonical homology directed repair (canonical HDR), homologous recombination (HR), alternative homology directed repair (Alt-HDR), double-strand break repair (DSBR), single-strand annealing (SSA), synthesis-dependent strand annealing (SDSA), break-induced replication (BIR), alternative end-joining (Alt-EJ), microhomology mediated end-joining (MMEJ), DNA synthesis-dependent microhomology-mediated end-joining (SD-MMEJ), canonical non-homologous end-joining repair (C-NHEJ), alternative non-homologous end joining (A-NHEJ), translesion DNA synthesis repair (TLS), base excision repair (BER), nucleotide excision repair (NER), mismatch repair (MMR), DNA damage responsive (DDR), blunt end joining, single strand break repair (SSBR), interstrand crosslink repair (ICL), Fanconi Anemia (FA) Pathway and combinations thereof.

24. The method of claim 16, wherein:
the at least one first DRP is homologous recombination (HR) and the at least one second DRP is one or more non-homologous end joining (NHEJ) DNA Repair pathway(s);
the at least one first DRP is an Alt-EJ pathway such as MMEJ, and the at least one second DRP is one or more non-homologous end joining (NHEJ) DNA Repair pathway(s);
the at least one first DRP is an Alt-EJ pathway such as MMEJ, and the at least one second DRP is the homologous recombination (HR) DNA Repair pathway(s); or
the at least one first DRP is an Alt-EJ pathway such as MMEJ, and the at least one second DRP is one or more alternative DNA repair pathway.

25. The method of claim 16, wherein the upmodulating comprises:
a) expressing, including causing overexpression of at least one component of the DRP in said cell,
b) introducing into said cell, at least one component of the said DRP, and/or
c) contacting said cell, with at least one stimulator such as a chemical stimulator of a component of the DRP, such as HR stimulators such as RS-1 and/or NHEJ stimulators, such as IP6.

26. The method of claim 16, wherein the downmodulating comprises:
a) contacting said cell, with at least one inhibitor such as a chemical inhibitor, such as NHEJ inhibitor selected from the group of NU7441, Olaparib, DNA Ligase IV inhibitor, Scr7 KU-0060648 anti-EGFR-antibody C225 (Cetuximab), Compound 401 (2-(4-Morpholinyl)-4H-pyrimido[2,I-a]isoquinolin-4-one), Vanillin, Wortmannin, DMNB, IC87361, LY294002, OK-1035, CO 15, NK314, PI 103 hydrochloride and combinations thereof, MMEJ inhibitors selected from the group of Mirin, derivatives of Mirin, inhibitors of PoIQ, inhibitors of CtIP and combinations thereof, HR inhibitors such as RI-1 and/or BO2, of a component of the DRP,
b) inactivating or downregulating at least one component of the DRP, by contacting or expressing in said cell, at least one inhibitory nucleic acid such as a miRNA, a siRNA, a shRNA, and/or
c) expressing in said cell a protein that inhibits the said DRP, or any combination thereof.

27. (canceled)

28. A kit for introducing at least one transgene into a cell comprising:
in one container a vector encoding a nuclease and/or nickase targeting at least one locus comprising an insertion site of an endogenous retrovirus (ERV) sequence or a LTR-retrotransposon (LTR-RT) sequence, such as SEQ ID NOs: 1 and 2, preferably a locus comprising (i) nucleotides 29021 to 40247 of SEQ ID NO: 1 or a sequence having 95%, 98% or 99% sequence identity with nucleotides 29021 to 40247 of SEQ ID NO: 1 and (ii) at least nucleotides 29020 to 31020 of SEQ ID NO: 2 or a sequence having 95% 98%, or 99% sequence identity with nucleotides 29020 to 31020 of SEQ ID NO: 2, or a locus comprising nucleotides 29521 to 39747 of SEQ ID NO: 1 or a sequence having 95%, 98% or 99% sequence identity with nucleotides 29521 to 4247 of SEQ ID NO: 1 and (ii) comprising nucleotides 29520 to 30520 of SEQ ID NO: 2 or a sequence having 95% 98%, or 99% sequence identity with nucleotides 29520 to 30520 of SEQ ID NO: 2, including an ERV sequence or a LTR-RT sequence integrated into the insertion site, such as SEQ ID NO: 3 and, optionally at least one vector encoding at least one targeting element guiding said least one nuclease and/or nickase,
optionally, in a separate container at least one vector encoding at least one targeting element guiding said least one nuclease and/or nickase,
in a separate container: at least one stimulator and/or inhibitor a DNA Repair Pathway (DRP), and/or
one or more vectors comprising one or more genes encoding one or more of the DRP proteins of Table 2 or SEQ ID Nos: 25-28, 38-58 and/or 59 or sequences having at least 80%, 85%, 90%, 95%, 98% or 99% sequence identity with SEQ ID Nos: 25-28, 38-58 and/or 59;
and instructions how to transfect the cell with the at least one transgene using the at least one nuclease and/or nickase and the at least one stimulator and/or inhibitor.
